SEQUENCE LISTING (358 sequences)

SEQ ID NO: 1 - protein, 8 aa, synthetic construct
  REGION 1..8: note = BGA113K HCDR1 (IMGT)
GYIFTSYY

SEQ ID NO: 2 - protein, 8 aa, synthetic construct
  REGION 1..8: note = BGA113K HCDR2 (IMGT)
INPQTGKT

SEQ ID NO: 3 - protein, 13 aa, synthetic construct
  REGION 1..13: note = BGA113K/BGA13 HCDR3 (IMGT)
AREYGNYNYPLDY

SEQ ID NO: 4 - protein, 6 aa, synthetic construct
  REGION 1..6: note = BGA113K LCDR1 (IMGT)
ENQYGY

SEQ ID NO: 5 - skipped

SEQ ID NO: 6 - protein, 9 aa, synthetic construct
  REGION 1..9: note = BGA113K LCDR3 (IMGT) or BGA113K LCDR3 (Kabat) or BGA113K LCDR3 (Chothia)
QHHLGTPYT

SEQ ID NO: 7 - protein, 5 aa, synthetic construct
  REGION 1..5: note = BGA113K HCDR1 (Kabat)
SYYLH

SEQ ID NO: 8 - protein, 17 aa, synthetic construct
  REGION 1..17: note = BGA113K HCDR2 (Kabat)
YINPQTGKTSYAQKFQG

SEQ ID NO: 9 - protein, 11 aa, synthetic construct
  REGION 1..11: note = BGA113K HCDR3 (Kabat) or BGA113K HCDR3 (Chothia) LCDR1 (Chothia)
EYGNYNYPLDY

SEQ ID NO: 10 - protein, 11 aa, synthetic construct
  REGION 1..11: note = BGA113K LCDR1 (Kabat) or BGA113K
RASENQYGYLA

SEQ ID NO: 11 - protein, 7 aa, synthetic construct
  REGION 1..7: note = BGA113K LCDR2 (Kabat)
NYKNLVE

SEQ ID NO: 12 - protein, 7 aa, synthetic construct
  REGION 1..7: note = BGA113K HCDR1 (Chothia
GYIFTSY

SEQ ID NO: 13 - protein, 6 aa, synthetic construct
  REGION 1..6: note = BGA113K HCDR2 (Chothia)
NPQTGK

SEQ ID NO: 14 - protein, 120 aa, synthetic construct
  REGION 1..120: note = BGA113K VH
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSS

SEQ ID NO: 15 - protein, 107 aa, synthetic construct
  REGION 1..107: note = BGA113/BGA113K VL
DIQMTQSPSSLSASVGDRVTITCRASENQYGYLAWYQQKPGKVPKLLIYNYKNLVEGVPSRFSGSGSGTDFTLTISSLQPEDVATYYCQHHLGTPYTFGQGTKVEIK

SEQ ID NO: 16 - DNA, 360 bp, synthetic construct
  misc_feature 1..360: note = BGA113K VH DNA
  source 1..360: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagc

SEQ ID NO: 17 - DNA, 321 bp, synthetic construct
  misc_feature 1..321: note = BGA113/BGA113K VL DNA
  source 1..321: mol_type = other DNA
gatattcagatgacccagagcccgagcagcctgagcgcgagcgtgggcgatcgcgtgaccattacctgccgcgcgagcgaaaaccagtatggctatctggcgtggtatcagcagaaaccgggcaaagtgccgaaactgctgatttataactataaaaacctggtggaaggcgtgccgagccgctttagcggcagcggcagcggcaccgattttaccctgaccattagcagcctgcagccggaagatgtggcgacctattattgccagcatcatctgggcaccccgtatacctttggccagggcaccaaagtggaaattaaa

SEQ ID NO: 18 - protein, 8 aa, synthetic construct
  REGION 1..8: note = BGA190
GFTFSSFG

SEQ ID NO: 19 - protein, 8 aa, synthetic construct
  REGION 1..8: note = BGA190 HCDR2 (IMGT)
ISIGSDII

SEQ ID NO: 20 - protein, 13 aa, synthetic construct
  REGION 1..13: note = BGA190 HCDR3 (IMGT)
TRRSYRSYWYFDV

SEQ ID NO: 21 - protein, 6 aa, synthetic construct
  REGION 1..6: note = BGA190 LCDR1 (IMGT)
ENIYSY

SEQ ID NO: 22 - skipped

SEQ ID NO: 23 - protein, 9 aa, synthetic construct
  REGION 1..9: note = BGA190 LCDR3 (IMGT) or BGA190 LCDR3 (Chothia)
QHHFLSPWM

SEQ ID NO: 24 - protein, 5 aa, synthetic construct
  REGION 1..5: note = BGA190 HCDR1 (Kabat)
SFGMH

SEQ ID NO: 25 - protein, 17 aa, synthetic construct
  REGION 1..17: note = BGA190 HCDR2 (Kabat)
YISIGSDIIYYADTVKG

SEQ ID NO: 26 - protein, 11 aa, synthetic construct
  REGION 1..11: note = BGA190 HCDR3 (Kabat) or BGA190 HCDR3 (Chothia)
RSYRSYWYFDV

SEQ ID NO: 27 - protein, 11 aa, synthetic construct
  REGION 1..11: note = BGA190 LCDR1 (Kabat) or BGA190 LCDR1 (Chothia)
RTSENIYSYLA

SEQ ID NO: 28 - protein, 7 aa, synthetic construct
  REGION 1..7: note = BGA190 LCDR2 (Kabat) or BGA190 LCDR2 (Chothia)
NAKTLAE

SEQ ID NO: 29 - protein, 7 aa, synthetic construct
  REGION 1..7: note = BGA190 HCDR1 (Chothia)
GFTFSSF

SEQ ID NO: 30 - protein, 6 aa, synthetic construct
  REGION 1..6: note = BGA190 HCDR2 (Chothia)
SIGSDI

SEQ ID NO: 31 - protein, 120 aa, synthetic construct
  REGION 1..120: note = BGA190 VH
DVQLVESGGGLVQPGGSRELSCAASGFTFSSFGMHWVRQAPERGLEWVAYISIGSDIIYYADTVKGRFTISRDNPKNTLFLQMTSLRSEDTAVYYCTRRSYRSYWYFDVWGAGTTVTVSS

SEQ ID NO: 32 - protein, 107 aa, synthetic construct
  REGION 1..107: note = BGA190 VL
DIQMTQSPASLSASVGETVTITCRTSENIYSYLAWYQQKQGKSPHLLVYNAKTLAEGVPSRFSGSGSGTQFSLKIISLQPEDFGTYYCQHHFLSPWMFGGGTKLEIK

SEQ ID NO: 33 - DNA, 360 bp, synthetic construct
  misc_feature 1..360: note = BGA190 VH DNA
  source 1..360: mol_type = other DNA
gatgtgcagctggtggagtctgggggaggcttagtgcagcctggagggtcccgggaactctcctgtgcagcctctggattcactttcagtagctttggaatgcactgggttcgtcaggctccagagagggggctggagtgggtcgcatacattagtattggcagtgatatcatctactatgcagacacagtgaagggccgattcaccatctccagagacaatcccaagaacaccctgttcctgcaaatgaccagtctgaggtctgaggacacggccgtgtattactgtacaagaaggtcttataggtcctactggtacttcgatgtctggggcgcagggaccacggtcaccgtctcctca

SEQ ID NO: 34 - DNA, 321 bp, synthetic construct
  misc_feature 1..321: note = BGA190 VL DNA
  source 1..321: mol_type = other DNA
gacatccagatgactcagtctccagcctccctatctgcatctgtgggagaaactgtcaccatcacatgtcgaacaagtgagaatatttacagttatttagcatggtatcagcagaaacagggaaaatctcctcatctcctggtctataatgcaaaaaccttagcagagggtgtgccatcaaggttcagtggcagtggatcaggcacacagttttctctgaagatcatcagcctgcagcctgaagattttgggacttattactgtcagcatcattttcttagtccgtggatgttcggtggaggcaccaagctggaaatcaaa

SEQ ID NO: 35 - protein, 8 aa, synthetic construct
  REGION 1..8: note = BGA288 HCDR1 (IMGT)
GYTFTDYN

SEQ ID NO: 36 - protein, 8 aa, synthetic construct
  REGION 1..8: note = BGA288 HCDR2 (IMGT)
IGPNYGGT

SEQ ID NO: 37 - protein, 12 aa, synthetic construct
  REGION 1..12: note = BGA288 HCDR3 (IMGT)
ARRGSIPRAVDY

SEQ ID NO: 38 - protein, 6 aa, synthetic construct
  REGION 1..6: note = BGA288 LCDR1 (IMGT)
QDIHNY

SEQ ID NO: 39 - skipped

SEQ ID NO: 40 - protein, 9 aa, synthetic construct
  REGION 1..9: note = BGA288 LCDR3 (IMGT) or BGA288 LCDR3 (Kabat) or BGA288 LCDR3 (Chothia)
LQYDEFPYT

SEQ ID NO: 41 - protein, 5 aa, synthetic construct
  REGION 1..5: note = BGA288 HCDR1 (Kabat)
DYNID

SEQ ID NO: 42 - protein, 17 aa, synthetic construct
  REGION 1..17: note = BGA288 HCDR2 (Kabat)
DIGPNYGGTIYNQKFKG

SEQ ID NO: 43 - protein, 10 aa, synthetic construct
  REGION 1..10: note = BGA288 HCDR3 (Kabat) or BGA288 HCDR3 (Chothia)
RGSIPRAVDY

SEQ ID NO: 44 - protein, 11 aa, synthetic construct
  REGION 1..11: note = BGA288 LCDR1 (Kabat) or BGA288 LCDR1 (Chothia)
KASQDIHNYLS

SEQ ID NO: 45 - protein, 7 aa, synthetic construct
  REGION 1..7: note = BGA288 LCDR2 (Kabat) or BGA288 LCDR2 (Chothia)
RANRLVD

SEQ ID NO: 46 - protein, 7 aa, synthetic construct
  REGION 1..7: note = BGA288 HCDR1 (Chothia)
GYTFTDY

SEQ ID NO: 47 - protein, 6 aa, synthetic construct
  REGION 1..6: note = BGA288 HCDR2 (Chothia)
GPNYGG

SEQ ID NO: 48 - protein, 119 aa, synthetic construct
  REGION 1..119: note = BGA288 VH
EVLLQQSGPELVKPGASVKIFCKASGYTFTDYNIDWVQQSHGKSLEWIGDIGPNYGGTIYNQKFKGKATLTVAKSSSTAYMELRSLTSEDTAVYYCARRGSIPRAVDYWGQGTSVTVSS

SEQ ID NO: 49 - protein, 107 aa, synthetic construct
  REGION 1..107: note = BGA288 VL
DIKMTQSPSSMYASLGERVTITCKASQDIHNYLSWFQQKPGKSPKTLIYRANRLVDGVPSRFSGSGSGQDYSLTISSLEYEDMGIYYCLQYDEFPYTFGGGTKLEIK

SEQ ID NO: 50 - DNA, 357 bp, synthetic construct
  misc_feature 1..357: note = BGA288 VH DNA
  source 1..357: mol_type = other DNA
gaggtcctgctgcaacagtctggacctgagctggtgaagcctggggcttcagtgaagattttctgtaaggcttctggatacacattcactgactacaacatagactgggtgcagcagagccatggaaagagccttgagtggattggagatattggtcctaattatggtggtactatctacaaccagaagttcaagggcaaggccacattgactgtagccaagtcctccagcacagcctacatggagctccgcagcctgacatctgaggacactgcagtctattactgtgcaagacgcggtagcatcccgagggctgtggactactggggtcaaggaacctcagtcaccgtctcctca

SEQ ID NO: 51 - DNA, 321 bp, synthetic construct
  misc_feature 1..321: note = BGA288 VL DNA
  source 1..321: mol_type = other DNA
gacatcaagatgacccagtctccatcttccatgtatgcatctctaggagagagagtcactatcacttgcaaggcgagtcaggacattcataactatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttccgtacacgttcggaggggggaccaagctggaaataaaa

SEQ ID NO: 52 - protein, 8 aa, synthetic construct
  REGION 1..8: note = BGA-7207 HCDR1 (IMGT)
GFRLDTTE

SEQ ID NO: 53 - protein, 8 aa, synthetic construct
  REGION 1..8: note = BGA-7207 HCDR2 (IMGT)
IMGISGST

SEQ ID NO: 54 - protein, 16 aa, synthetic construct
  REGION 1..16: note = BGA-7207 HCDR3 (IMGT)
ARVVDSLFDDSAVFDY

SEQ ID NO: 55 - protein, 5 aa, synthetic construct
  REGION 1..5: note = BGA-7207 HCDR1 (Kabat)
TTEVG

SEQ ID NO: 56 - protein, 17 aa, synthetic construct
  REGION 1..17: note = BGA-7207 HCDR2 (Kabat)
TIMGISGSTYYADSVKG

SEQ ID NO: 57 - protein, 14 aa, synthetic construct
  REGION 1..14: note = BGA-7207 HCDR3 (Kabat) or BGA-7207 HCDR3 (Chothia)
VVDSLFDDSAVFDY

SEQ ID NO: 58 - protein, 7 aa, synthetic construct
  REGION 1..7: note = BGA-7207 HCDR1 (Chothia)
GFRLDTT

SEQ ID NO: 59 - protein, 6 aa, synthetic construct
  REGION 1..6: note = BGA-7207 HCDR2 (Chothia)
MGISGS

SEQ ID NO: 60 - protein, 123 aa, synthetic construct
  REGION 1..123: note = BGA-7207 VH
EVQLLESGGGLVQPGGSLRLSCAASGFRLDTTEVGWVRQAPGKGLEWVSTIMGISGSTYYADSVKGRFTISRDNSKNTLYLQMNSLRAEDTAVYYCARVVDSLFDDSAVFDYWGQGTLVTVSS

SEQ ID NO: 61 - DNA, 369 bp, synthetic construct
  misc_feature 1..369: note = BGA-7207 VH DNA
  source 1..369: mol_type = other DNA
gaggtccagttacttgagagtggtggaggtctggtccaaccaggaggttcgctgcgtttatcctgcgccgcgtctggattcagattggacaccaccgaagtaggttgggtgcgtcaagcgccggggaaaggactggaatgggtctccaccatcatgggtattagtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtcgattccctctttgatgacagcgccgtttttgattactggggacagggcaccttagttacagtctcatcg

SEQ ID NO: 62 - protein, 8 aa, synthetic construct
  REGION 1..8: note = BGA-4712 HCDR1 (IMGT)
GFMLSAED

SEQ ID NO: 63 - protein, 8 aa, synthetic construct
  REGION 1..8: note = BGA-4712 HCDR2 (IMGT) or BGA-4712-M3 HCDR2 (IMGT)
ILDFGGST

SEQ ID NO: 64 - protein, 15 aa, synthetic construct
  REGION 1..15: note = BGA-4712 HCDR3 (IMGT) or BGA-4712-M3 HCDR3 (IMGT)
ARVVYHAGGGVTFDY

SEQ ID NO: 65 - protein, 5 aa, synthetic construct
  REGION 1..5: note = BGA-4712 HCDR1 (Kabat) or BGA-4712-M3 HCDR1 (Kabat)
AEDVG

SEQ ID NO: 66 - protein, 17 aa, synthetic construct
  REGION 1..17: note = BGA-4712 HCDR2 (Kabat)
AILDFGGSTYYADSVKG

SEQ ID NO: 67 - protein, 13 aa, synthetic construct
  REGION 1..13: note = BGA-4712/BGA-4712-M3 HCDR3 (Kabat) or BGA-4712/BGA-4712-M3 HCDR3 (Chothia)
VVYHAGGGVTFDY

SEQ ID NO: 68 - protein, 7 aa, synthetic construct
  REGION 1..7: note = BGA-4712 HCDR1 (Chothia)
GFMLSAE

SEQ ID NO: 69 - protein, 6 aa, synthetic construct
  REGION 1..6: note = BGA-4712 HCDR2 (Chothia)
LDFGGS

SEQ ID NO: 70 - protein, 122 aa, synthetic construct
  REGION 1..122: note = BGA-4712 VH
EVQLLESGGGLVQPGGSLRLSCAASGFMLSAEDVGWVRQAPGKGLEWVSAILDFGGSTYYADSVKGRFTISRDNSKNTLYLQMNSLRAEDTAVYYCARVVYHAGGGVTFDYWGQGTLVTVSS

SEQ ID NO: 71 - DNA, 366 bp, synthetic construct
  misc_feature 1..366: note = BGA-4712 VH DNA
  source 1..366: mol_type = other DNA
gaggtccagttacttgagagtggtggaggtctggtccaaccaggaggttcgctgcgtttatcctgcgccgcgtctggattcatgttgtccgccgaagacgtgggttgggtgcgtcaagcgccggggaaaggactggaatgggtctccgccatcttggattttggtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtctaccatgctggtggtggcgtcacctttgattactggggacagggcaccttagttacagtctcatcg

SEQ ID NO: 72 - protein, 8 aa, synthetic construct
  REGION 1..8: note = BGA-4712-M3 HCDR1 (IMGT)
GFTLSAED

SEQ ID NO: 73 - protein, 17 aa, synthetic construct
  REGION 1..17: note = BGA-4712-M3 HCDR2 (Kabat)
AILDFGGSTYYAESVKG

SEQ ID NO: 74 - protein, 7 aa, synthetic construct
  REGION 1..7: note = BGA-4712-M3 HCDR1 (Chothia)
GFTLSAE

SEQ ID NO: 75 - protein, 122 aa, synthetic construct
  REGION 1..122: note = BGA-4712-M3 VH
EVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYWGQGTLVTVSS

SEQ ID NO: 76 - DNA, 366 bp, synthetic construct
  misc_feature 1..366: note = BGA-4712-M3 VH DNA
  source 1..366: mol_type = other DNA
gaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattactggggacagggcaccttagttacagtctcatcg

SEQ ID NO: 77 - protein, 8 aa, synthetic construct
  REGION 1..8: note = BGA-5623 HCDR1 (IMGT)
GFTVSAED

SEQ ID NO: 78 - protein, 8 aa, synthetic construct
  REGION 1..8: note = BGA-5623 HCDR2 (IMGT)
ILDKGGST

SEQ ID NO: 79 - protein, 15 aa, synthetic construct
  REGION 1..15: note = BGA-5623 HCDR3 (IMGT)
ARIVYHAGGGVTFDT

SEQ ID NO: 80 - protein, 17 aa, synthetic construct
  REGION 1..17: note = BGA-5623 HCDR2 (Kabat)
AILDKGGSTYYAESVKG

SEQ ID NO: 81 - protein, 13 aa, synthetic construct
  REGION 1..13: note = BGA-5623 HCDR3 (Kabat/Chothia)
IVYHAGGGVTFDT

SEQ ID NO: 82 - protein, 7 aa, synthetic construct
  REGION 1..7: note = BGA-5623 HCDR1 (Chothia)
GFTVSAE

SEQ ID NO: 83 - protein, 6 aa, synthetic construct
  REGION 1..6: note = BGA-5623 HCDR2 (Chothia)
LDKGGS

SEQ ID NO: 84 - protein, 122 aa, synthetic construct
  REGION 1..122: note = BGA-5623 VH
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSS

SEQ ID NO: 85 - DNA, 366 bp, synthetic construct
  misc_feature 1..366: note = BGA-5623 VH DNA
  source 1..366: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 86 - protein, 122 aa, synthetic construct
  REGION 1..122: note = BGA-7556 VH
EVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYRGQGTQVTVSS

SEQ ID NO: 87 - DNA, 366 bp, synthetic construct
  misc_feature 1..366: note = BGA-7556 VH DNA
  source 1..366: mol_type = other DNA
gaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattaccggggacagggcacccaagttacagtctcatcg

SEQ ID NO: 88 - protein, 702 aa, synthetic construct
  REGION 1..702: note = full-length human CEA
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 89 - protein, 705 aa, synthetic construct
  REGION 1..705: note = Macaca CEA
MGSPSAPLHRWCIPWQTLLLTASLLTFWNPPTTAQLTIESRPFNVAEGKEVLLLAHNVSQNLFGYIWYKGERVDASRRIGSCVIRTQQITPGPAHSGRETIDFNASLLIHNVTQSDTGSYTIQVIKEDLVNEEATGQFRVYPELPKPYISSNNSNPVEDKDAVALTCEPETQDTTYLWWVNNQSLPVSPRLELSSDNRTLTVFNIPRNDTTSYKCETQNPVSVRRSDPVTLNVLYGPDAPTISPLNTPYRAGENLNLTCHAASNPTAQYFWFVNGTFQQSTQELFIPNITVNNSGSYMCQAHNSATGLNRTTVTAITVYAELPKPYITSNNSNPIEDKDAVTLTCEPETQDTTYLWWVNNQSLSVSSRLELSNDNRTLTVFNIPRNDTTFYECETQNPVSVRRSDPVTLNVLYGPDAPTISPLNTPYRAGENLNLSCHAASNPAAQYSWFVNGTFQQSTQELFIPNITVNNSGSYMCQAHNSATGLNRTTVTAITVYVELPKPYISSNNSNPIEDKDAVTLTCEPVAENTTYLWWVNNQSLSVSPRLQLSNGNRILTLLSVTRNDTGPYECGIQNSESAKRSDPVTLNVTYGPDTPIISPPDLSYRSGANLNLSCHSDSNPSPQYSWLINGTLRQHTQVLFISKITSNNSGAYACFVSNLATGRNNSIVKNISVSSGDSAPGSSGLSARATVGIIIGMLVGVALM

SEQ ID NO: 90 - protein, 344 aa, synthetic construct
  REGION 1..344: note = full-length human CEACAM6
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI

SEQ ID NO: 91 - protein, 693 aa, synthetic construct
  REGION 1..693: note = 1-687 of huCEA
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGAHHHHHH

SEQ ID NO: 92 - protein, 696 aa, synthetic construct
  REGION 1..696: note = 1-690 of monkey CEA
MGSPSAPLHRWCIPWQTLLLTASLLTFWNPPTTAQLTIESRPFNVAEGKEVLLLAHNVSQNLFGYIWYKGERVDASRRIGSCVIRTQQITPGPAHSGRETIDFNASLLIHNVTQSDTGSYTIQVIKEDLVNEEATGQFRVYPELPKPYISSNNSNPVEDKDAVALTCEPETQDTTYLWWVNNQSLPVSPRLELSSDNRTLTVFNIPRNDTTSYKCETQNPVSVRRSDPVTLNVLYGPDAPTISPLNTPYRAGENLNLTCHAASNPTAQYFWFVNGTFQQSTQELFIPNITVNNSGSYMCQAHNSATGLNRTTVTAITVYAELPKPYITSNNSNPIEDKDAVTLTCEPETQDTTYLWWVNNQSLSVSSRLELSNDNRTLTVFNIPRNDTTFYECETQNPVSVRRSDPVTLNVLYGPDAPTISPLNTPYRAGENLNLSCHAASNPAAQYSWFVNGTFQQSTQELFIPNITVNNSGSYMCQAHNSATGLNRTTVTAITVYVELPKPYISSNNSNPIEDKDAVTLTCEPVAENTTYLWWVNNQSLSVSPRLQLSNGNRILTLLSVTRNDTGPYECGIQNSESAKRSDPVTLNVTYGPDTPIISPPDLSYRSGANLNLSCHSDSNPSPQYSWLINGTLRQHTQVLFISKITSNNSGAYACFVSNLATGRNNSIVKNISVSSGDSAPGSSGLSARAHHHHHH

SEQ ID NO: 93 - protein, 326 aa, synthetic construct
  REGION 1..326: note = 1-320 of CEACAM6
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPSKANYRPGENLNLSCHAASNPPAQYSWFINGTFQQSTQELFIPNITVNNSGSYMCQAHNSATGLNRTTVTMITVSGHHHHHH

SEQ ID NO: 94 - protein, 115 aa, synthetic construct
  REGION 1..115: note = 1-78 of huCEA
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQN

SEQ ID NO: 95 - protein, 290 aa, synthetic construct
  REGION 1..290: note = 398-687 of huCEA
ELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGA

SEQ ID NO: 96 - protein, 411 aa, synthetic construct
  REGION 1..411: note = CEA-v
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGAHHHHHH

SEQ ID NO: 97 - protein, 243 aa, synthetic construct
  REGION 1..243: note = 1-273 of CEACAM6
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTIS

SEQ ID NO: 98 - protein, 92 aa, synthetic construct
  REGION 1..92: note = 596-687 of huCEA
PIISPPDSSYLSGANLNLSCHSASNPSPQYSWRINGIPQQHTQVLFIAKITPNNNGTYACFVSNLATGRNNSIVKSITVSASGTSPGLSAGA

SEQ ID NO: 99 - protein, 341 aa, synthetic construct
  REGION 1..341: note = CHIM
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPIISPPDSSYLSGANLNLSCHSASNPSPQYSWRINGIPQQHTQVLFIAKITPNNNGTYACFVSNLATGRNNSIVKSITVSASGTSPGLSAGAHHHHHH

SEQ ID NO: 100 - protein, 464 aa, synthetic construct
  REGION 1..464: note = CEACAM1
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGSSGPLQ

SEQ ID NO: 101 - protein, 252 aa, synthetic construct
  REGION 1..252: note = CEACAM3
MGPPSASPHRECIPWQGLLLTASLLNFWNPPTTAKLTIESMPLSVAEGKEVLLLVHNLPQHLFGYSWYKGERVDGNSLIVGYVIGTQQATPGAAYSGRETIYTNASLLIQNVTQNDIGFYTLQVIKSDLVNEEATGQFHVYQENAPGLPVGAVAGIVTGVLVGVALVAALVCFLLLAKTGRTSIQRDLKEQQPQALAPGRGPSHSSAFSMSPLSTAQAPLPNPRTAASIYEELLKHDTNIYCRMDHKAEVAS

SEQ ID NO: 102 - protein, 265 aa, synthetic construct
  REGION 1..265: note = CEACAM7
MGSPSACPYRVCIPWQGLLLTASLLTFWNLPNSAQTNIDVVPFNVAEGKEVLLVVHNESQNLYGYNWYKGERVHANYRIIGYVKNISQENAPGPAHNGRETIYPNGTLLIQNVTHNDAGIYTLHVIKENLVNEEVTRQFYVFSEPPKPSITSNNFNPVENKDIVVLTCQPETQNTTYLWWVNNQSLLVSPRLLLSTDNRTLVLLSATKNDIGPYECEIQNPVGASRSDPVTLNVRYESVQASSPDLSAGTAVSIMIGVLAGMALI

SEQ ID NO: 103 - protein, 349 aa, synthetic construct
  REGION 1..349: note = CEACAM8
MGPISAPSCRWRIPWQGLLLTASLFTFWNPPTTAQLTIEAVPSNAAEGKEVLLLVHNLPQDPRGYNWYKGETVDANRRIIGYVISNQQITPGPAYSNRETIYPNASLLMRNVTRNDTGSYTLQVIKLNLMSEEVTGQFSVHPETPKPSISSNNSNPVEDKDAVAFTCEPETQNTTYLWWVNGQSLPVSPRLQLSNGNRTLTLLSVTRNDVGPYECEIQNPASANFSDPVTLNVLYGPDAPTISPSDTYYHAGVNLNLSCHAASNPPSQYSWSVNGTFQQYTQKLFIPNITTKNSGSYACHTTNSATGRNRTTVRMITVSDALVQGSSPGLSARATVSIMIGVLARVALI

SEQ ID NO: 104 - DNA, 2109 bp, synthetic construct
  misc_feature 1..2109: note = full-length human CEA DNA
  source 1..2109: mol_type = other DNA
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatag

SEQ ID NO: 105 - DNA, 2118 bp, synthetic construct
  misc_feature 1..2118: note = monkey CEA DNA
  source 1..2118: mol_type = other DNA
atggggtctccctcagcccctcttcacagatggtgcatcccctggcagacgctcctgctcacagcctcacttctaaccttctggaacccgcccaccactgcccagctcactattgaatccaggccgttcaatgttgcagaggggaaggaggttcttctacttgcccacaatgtgtcccagaatctttttggctacatttggtacaagggagaaagagtggatgccagccgtcgaattggatcatgtgtaataagaactcaacaaattaccccagggcccgcacacagcggtcgagagacaatagacttcaatgcatccctgctgatccacaatgtcacccagagtgacacaggatcctacaccatacaagtcataaaggaagatcttgtgaatgaagaagcaactggccagttccgggtatacccggagctgcccaagccctacatctccagcaacaactccaaccccgtggaggacaaggatgctgtggccttaacctgtgaacctgagactcaggacacaacctacctgtggtgggtaaacaatcagagcctcccggtcagtcccaggctggagctgtccagtgacaacaggaccctcactgtattcaatattccaagaaatgacacaacatcctacaaatgtgaaacccagaacccagtgagtgtcagacgcagcgacccagtcaccctgaacgtcctctatggcccggatgcgcccaccatttcccctctaaacacaccttacagagcaggggaaaatctgaacctcacctgccacgcagcctctaacccaactgcacagtacttttggtttgtcaatgggacgttccagcaatccacacaagagctctttatacccaacatcaccgtgaataatagcggatcctatatgtgccaagcccataactcagccactggcctcaataggaccacagtcacggcgatcacagtctacgcggagctgcccaagccctacatcaccagcaacaactccaaccccatagaggacaaggatgctgtgaccttaacctgtgaacctgagactcaggacacaacctacctgtggtgggtaaacaatcagagcctctcggtcagttccaggctggagctgtccaatgacaacaggaccctcactgtattcaatattccaagaaacgacacaacgttctacgaatgtgagacccagaacccagtgagtgtcagacgcagcgacccagtcaccctgaatgtcctctatggcccggatgcgcccaccatttcccctctaaacacaccttacagagcaggggaaaatctgaacctctcctgccacgcagcctctaacccagctgcacagtactcttggtttgtcaatgggacgttccagcaatccacacaagagctctttatacccaacatcaccgtgaataatagcggatcctatatgtgccaagcccataactcagccactggcctcaataggaccacagtcacggcgatcacagtctatgtggagctgcccaagccctacatctccagcaacaactccaaccccatagaggacaaggatgctgtgaccttaacctgtgaacctgtggctgagaacacaacctacctgtggtgggtaaacaatcagagcctctcggtcagtcccaggctgcagctctccaatggcaacaggatcctcactctactcagtgtcacacggaatgacacaggaccctatgaatgtggaatccagaactcagagagtgcaaaacgcagtgacccagtcaccctgaatgtcacctatggcccagacacccccatcatatcccccccagacttgtcttaccgttcgggagcaaacctcaacctctcctgccactcggactctaacccatccccgcagtattcttggcttatcaatgggacactgcggcaacacacacaagttctctttatctccaaaatcacatcaaacaatagcggggcctatgcctgttttgtctctaacttggctaccggtcgcaataactccatagtcaagaacatctcagtctcctctggcgattcagcacctggaagttctggtctctcagctagggctactgtcggcatcataattggaatgctggttggggttgctctgatgtag

SEQ ID NO: 106 - DNA, 1035 bp, synthetic construct
  misc_feature 1..1035: note = full-length human CEACAM6 DNA
  source 1..1035: mol_type = other DNA
atgggacccccctcagcccctccctgcagattgcatgtcccctggaaggaggtcctgctcacagcctcacttctaaccttctggaacccacccaccactgccaagctcactattgaatccacgccgttcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttccccctcaaaggccaattaccgtccaggggaaaatctgaacctctcctgccacgcagcctctaacccacctgcacagtactcttggtttatcaatgggacgttccagcaatccacacaagagctctttatccccaacatcactgtgaataatagcggatcctatatgtgccaagcccataactcagccactggcctcaataggaccacagtcacgatgatcacagtctctggaagtgctcctgtcctctcagctgtggccaccgtcggcatcacgattggagtgctggccagggtggctctgatatag

SEQ ID NO: 107 - DNA, 2079 bp, synthetic construct
  misc_feature 1..2079: note = 1-687 of huCEA DNA
  source 1..2079: mol_type = other DNA
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggcccatcaccatcaccatcac

SEQ ID NO: 108 - DNA, 2088 bp, synthetic construct
  misc_feature 1..2088: note = 1-690 of monkey CEA DNA
  source 1..2088: mol_type = other DNA
atggggtctccctcagcccctcttcacagatggtgcatcccctggcagacgctcctgctcacagcctcacttctaaccttctggaacccgcccaccactgcccagctcactattgaatccaggccgttcaatgttgcagaggggaaggaggttcttctacttgcccacaatgtgtcccagaatctttttggctacatttggtacaagggagaaagagtggatgccagccgtcgaattggatcatgtgtaataagaactcaacaaattaccccagggcccgcacacagcggtcgagagacaatagacttcaatgcatccctgctgatccacaatgtcacccagagtgacacaggatcctacaccatacaagtcataaaggaagatcttgtgaatgaagaagcaactggccagttccgggtatacccggagctgcccaagccctacatctccagcaacaactccaaccccgtggaggacaaggatgctgtggccttaacctgtgaacctgagactcaggacacaacctacctgtggtgggtaaacaatcagagcctcccggtcagtcccaggctggagctgtccagtgacaacaggaccctcactgtattcaatattccaagaaatgacacaacatcctacaaatgtgaaacccagaacccagtgagtgtcagacgcagcgacccagtcaccctgaacgtcctctatggcccggatgcgcccaccatttcccctctaaacacaccttacagagcaggggaaaatctgaacctcacctgccacgcagcctctaacccaactgcacagtacttttggtttgtcaatgggacgttccagcaatccacacaagagctctttatacccaacatcaccgtgaataatagcggatcctatatgtgccaagcccataactcagccactggcctcaataggaccacagtcacggcgatcacagtctacgcggagctgcccaagccctacatcaccagcaacaactccaaccccatagaggacaaggatgctgtgaccttaacctgtgaacctgagactcaggacacaacctacctgtggtgggtaaacaatcagagcctctcggtcagttccaggctggagctgtccaatgacaacaggaccctcactgtattcaatattccaagaaacgacacaacgttctacgaatgtgagacccagaacccagtgagtgtcagacgcagcgacccagtcaccctgaatgtcctctatggcccggatgcgcccaccatttcccctctaaacacaccttacagagcaggggaaaatctgaacctctcctgccacgcagcctctaacccagctgcacagtactcttggtttgtcaatgggacgttccagcaatccacacaagagctctttatacccaacatcaccgtgaataatagcggatcctatatgtgccaagcccataactcagccactggcctcaataggaccacagtcacggcgatcacagtctatgtggagctgcccaagccctacatctccagcaacaactccaaccccatagaggacaaggatgctgtgaccttaacctgtgaacctgtggctgagaacacaacctacctgtggtgggtaaacaatcagagcctctcggtcagtcccaggctgcagctctccaatggcaacaggatcctcactctactcagtgtcacacggaatgacacaggaccctatgaatgtggaatccagaactcagagagtgcaaaacgcagtgacccagtcaccctgaatgtcacctatggcccagacacccccatcatatcccccccagacttgtcttaccgttcgggagcaaacctcaacctctcctgccactcggactctaacccatccccgcagtattcttggcttatcaatgggacactgcggcaacacacacaagttctctttatctccaaaatcacatcaaacaatagcggggcctatgcctgttttgtctctaacttggctaccggtcgcaataactccatagtcaagaacatctcagtctcctctggcgattcagcacctggaagttctggtctctcagctagggctcatcaccatcaccatcac

SEQ ID NO: 109 - DNA, 978 bp, synthetic construct
  misc_feature 1..978: note = 1-320 of CEACAM6 DNA
  source 1..978: mol_type = other DNA
atgggacccccctcagcccctccctgcagattgcatgtcccctggaaggaggtcctgctcacagcctcacttctaaccttctggaacccacccaccactgccaagctcactattgaatccacgccgttcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttccccctcaaaggccaattaccgtccaggggaaaatctgaacctctcctgccacgcagcctctaacccacctgcacagtactcttggtttatcaatgggacgttccagcaatccacacaagagctctttatccccaacatcactgtgaataatagcggatcctatatgtgccaagcccataactcagccactggcctcaataggaccacagtcacgatgatcacagtctctggacatcaccatcaccatcac

SEQ ID NO: 110 - DNA, 345 bp, synthetic construct
  misc_feature 1..345: note = 1-78 of huCEA DNA
  source 1..345: mol_type = other DNA
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaat

SEQ ID NO: 111 - DNA, 870 bp, synthetic construct
  misc_feature 1..870: note = 398-687 of huCEA DNA
  source 1..870: mol_type = other DNA
gaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggcc

SEQ ID NO: 112 - DNA, 1233 bp, synthetic construct
  misc_feature 1..1233: note = CEA-v DNA
  source 1..1233: mol_type = other DNA
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggcccatcaccatcaccatcac

SEQ ID NO: 113 - DNA, 729 bp, synthetic construct
  misc_feature 1..729: note = 1-273 of NCA DNA
  source 1..729: mol_type = other DNA
atgggacccccctcagcccctccctgcagattgcatgtcccctggaaggaggtcctgctcacagcctcacttctaaccttctggaacccacccaccactgccaagctcactattgaatccacgccgttcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttcc

SEQ ID NO: 114 - DNA, 276 bp, synthetic construct
  misc_feature 1..276: note = 596-687 of huCEA DNA
  source 1..276: mol_type = other DNA
cccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggcc

SEQ ID NO: 115 - DNA, 1023 bp, synthetic construct
  misc_feature 1..1023: note = CHIM DNA
  source 1..1023: mol_type = other DNA
atgggacccccctcagcccctccctgcagattgcatgtcccctggaaggaggtcctgctcacagcctcacttctaaccttctggaacccacccaccactgccaagctcactattgaatccacgccgttcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttcccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggcccatcaccatcaccatcac

SEQ ID NO: 116 - protein, 8 aa, synthetic construct
  REGION 1..8: note = BGA13 HCDR1 (IMGT)
GYIFTSYW

SEQ ID NO: 117 - protein, 8 aa, synthetic construct
  REGION 1..8: note = BGA13 HCDR2 (IMGT)
INPNTGYT

SEQ ID NO: 118 - protein, 6 aa, synthetic construct
  REGION 1..6: note = BGA13 LCDR1 (IMGT)
ENIYGY

SEQ ID NO: 119 - skipped

SEQ ID NO: 120 - protein, 9 aa, synthetic construct
  REGION 1..9: note = BGA13 LCDR3 (IMGT)
QHHYGTPYT

SEQ ID NO: 121 - protein, 120 aa, synthetic construct
  REGION 1..120: note = BGA13 VH
QVQLQQSGAELAKSGASVKMSCKASGYIFTSYWLHWVKQRPGQGLEWIGYINPNTGYTNYSQKFKDKATLTADKSSSTAYMQLSSLTSEDSAVYYCAREYGNYNYPLDYWGQGTSVTVSS

SEQ ID NO: 122 - protein, 107 aa, synthetic construct
  REGION 1..107: note = BGA13 VL
DIQMTQSPASLSASVGETVTITCRASENIYGYLAWYQQKQGKSPQLLVFNAKNLVEGVPSRFSGSGSGTQFSLKINSLQPEDFGSYYCQHHYGTPYTFGGGTKLEIK

SEQ ID NO: 123 - protein, 330 aa, synthetic construct [sequence omitted]

SEQ ID NO: 124 - protein, 107 aa, synthetic construct [sequence omitted]

SEQ ID NO: 125 - protein, 450 aa, synthetic construct
  REGION 1..450: note = BGA131 heavy chain
QVQLVQSGAEVKKPGASVKVSCKASGYIFTSYWLHWVRQAPGQGLEWIGYINPNTGYTNYSQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 214 aa, synthetic construct
  REGION 1..214: note = BGA131 light chain
DIQMTQSPSSLSASVGDRVTITCRASENIYGYLAWYQQKPGKVPKLLIYNAKNLVEGVPSRFSGSGSGTDFTLTISSLQPEDVATYYCQHHYGTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 120 aa, synthetic construct
  REGION 1..120: note = BGA131 VH
QVQLVQSGAEVKKPGASVKVSCKASGYIFTSYWLHWVRQAPGQGLEWIGYINPNTGYTNYSQKFQGRVTMTRDTSTSTVYMELSSLRSEDTAVYYCAREYGNYNYPLDYWGQGTLVTVSS

SEQ ID NO: 128 - protein, 107 aa, synthetic construct
  REGION 1..107: note = BGA131 VL
DIQMTQSPSSLSASVGDRVTITCRASENIYGYLAWYQQKPGKVPKLLIYNAKNLVEGVPSRFSGSGSGTDFTLTISSLQPEDVATYYCQHHYGTPYTFGQGTKVEIK

SEQ ID NO: 129 - protein, 107 aa, synthetic construct; identical to SEQ ID NO: 128
  REGION 1..107: note = BGA13-1 VL

SEQ ID NO: 130 - protein, 120 aa, synthetic construct
  REGION 1..120: note = BGA13-1F VH
QVQLVQSGAEVKKPGASVKVSCKASGYIFTSYWLHWVRQAPGQGLEWIGYINPQTGYTSYAQKFQGRVTMTRDTSTSTVYMELSSLRSEDTAVYYCAREYGNYNYPLDYWGQGTLVTVSS

SEQ ID NO: 131 - protein, 107 aa, synthetic construct
  REGION 1..107: note = BGA13-1F-ph-L VL
DIQMTQSPSSLSASVGDRVTITCRASENQYGYLAWYQQKPGKVPKLLIYNFKNLVEGVPSRFSGSGSGTDFTLTISSLQPEDVATYYCQHHLGTPYTFGQGTKVEIK

SEQ ID NO: 132 - protein, 107 aa, synthetic construct
  REGION 1..107: note = BGA13-1F-ph-M VL
DIQMTQSPSSLSASVGDRVTITCRASENQYGYLAWYQQKPGKVPKLLIYNTKNLVEGVPSRFSGSGSGTDFTLTISSLQPEDVATYYCQHHLGTPYTFGQGTKVEIK

SEQ ID NO: 133 - protein, 120 aa, synthetic construct
  REGION 1..120: note = BGA113 VH
QVQLVQSGAEVKKPGASVKVSCKASGYIFTSYYLHWVRQAPGQGLEWIGYINPQTGYTSYAQKFQGRVTMTRDTSTSTVYMELSSLRSEDTAVYYCAREYGNYNYPLDYWGQGTLVTVSS

SEQ ID NO: 134 - DNA, 360 bp, synthetic construct
  misc_feature 1..360: note = BGA113 VH DNA
  source 1..360: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggctataccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagc

SEQ ID NO: 135 - protein, 255 aa, synthetic construct
  REGION 1..255: note = human CD137 FL AA
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 136 - DNA, 765 bp, synthetic construct
  misc_feature 1..765: note = human CD137 FL DNA
  source 1..765: mol_type = other DNA
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 137 - protein, 160 aa, synthetic construct
  REGION 1..160: note = human CD137 AA 24-183
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGH

SEQ ID NO: 138 - DNA, 480 bp, synthetic construct
  misc_feature 1..480: note = human CD137 24-183 DNA
  source 1..480: mol_type = other DNA
ctgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacac

SEQ ID NO: 139 - protein, 183 aa, synthetic construct
  REGION 1..183: note = human CD137 -his
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHHHHHHHGGGLNDIFEAQKIEWHE

SEQ ID NO: 140 - DNA, 549 bp, synthetic construct
  misc_feature 1..549: note = human CD137 -his DNA
  source 1..549: mol_type = other DNA
ctgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacaccatcaccatcaccatcacggaggcggtctgaacgacatcttcgaggctcagaaaatcgaatggcacgaa

SEQ ID NO: 141 - protein, 393 aa, synthetic construct
  REGION 1..393: note = human CD137 ECD-mIgG2a AA
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 142 - DNA, 1179 bp, synthetic construct
  misc_feature 1..1179: note = human CD137 ECD-mIgG2a DNA
  source 1..1179: mol_type = other DNA
ctgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacacgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 143 - protein, 233 aa, synthetic construct
  REGION 1..233: note = mIgG2a AA
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 144 - DNA, 699 bp, synthetic construct
  misc_feature 1..699: note = mIgG2a DNA
  source 1..699: mol_type = other DNA
gagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 145 - protein, 254 aa, synthetic construct
  REGION 1..254: note = human CD137 ligand AA
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 146 - DNA, 765 bp, synthetic construct
  misc_feature 1..765: note = human CD137 ligand cDNA
  source 1..765: mol_type = other DNA
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 147 - protein, 184 aa, synthetic construct
  REGION 1..184: note = human CD137 ligand 71-254 AA
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 148 - DNA, 552 bp, synthetic construct
  misc_feature 1..552: note = human CD137 ligand 71-254 DNA
  source 1..552: mol_type = other DNA
cgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 149 - protein, 422 aa, synthetic construct
  REGION 1..422: note = human CD137 ligand-mIgG2a AA
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 150 - DNA, 1266 bp, synthetic construct
  misc_feature 1..1266: note = human CD137 ligand-mIgG2a DNA
  source 1..1266: mol_type = other DNA
gagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaggtggaggcggttcacgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 151 - protein, 254 aa, synthetic construct
  REGION 1..254: note = cyno CD137 FL AA
MGNSCYNIVATLLLVLNFERTRSLQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQIIFFLALTSTVVLFLLFFLVLRFSVVKRSRKKLLYIFKQPFMRPVQTTQEEDGCSCRFPEEEEGGCEL

SEQ ID NO: 152 - DNA, 765 bp, synthetic construct
  misc_feature 1..765: note = cyno CD137 FL DNA
  source 1..765: mol_type = other DNA
atgggaaacagctgttacaacatagtagccactctgttgctggtcctgaactttgagaggacaagatccttgcaggatctgtgtagtaactgcccagctggtacattctgtgataataacaggagtcagatttgcagtccctgtcctccaaatagtttctccagcgcaggtggacaaaggacctgtgacatatgcaggcagtgtaaaggtgttttcaagaccaggaaggagtgttcctccaccagcaatgcagagtgtgactgcatttcaggatatcactgcctgggggcagaatgcagcatgtgtgaacaggattgtaaacaaggtcaagaattgacaaaaaaaggttgtaaagactgttgctttgggacatttaatgaccagaaacgtggcatctgtcgaccctggacaaactgttctttggatggaaagtctgtgcttgtgaatgggacgaaggagagggacgtggtctgcggaccatctccagccgacctctctccaggagcatcctctgcgaccccgcctgcccctgcgagagagccaggacactctccgcagatcatcttctttcttgcgctgacttcgactgtggtactcttcctgctgttcttcctcgtgctccgtttctctgttgttaaacggagcagaaagaaactcctgtatatattcaaacaaccatttatgagaccggtacaaaccactcaagaggaagatggatgtagctgccgatttccagaagaagaagaaggaggatgtgaactgtga

SEQ ID NO: 153 - protein, 163 aa, synthetic construct
  REGION 1..163: note = cyno CD137 ECD 24-186 AA
LQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQ

SEQ ID NO: 154 - DNA, 489 bp, synthetic construct
  misc_feature 1..489: note = cyno CD137 ECD 24-186 DNA
  source 1..489: mol_type = other DNA
ctgcaggatctgtgtagtaactgcccagctggtacattctgtgataataacaggagtcagatttgcagtccctgtcctccaaatagtttctccagcgcaggtggacaaaggacctgtgacatatgcaggcagtgtaaaggtgttttcaagaccaggaaggagtgttcctccaccagcaatgcagagtgtgactgcatttcaggatatcactgcctgggggcagaatgcagcatgtgtgaacaggattgtaaacaaggtcaagaattgacaaaaaaaggttgtaaagactgttgctttgggacatttaatgaccagaaacgtggcatctgtcgaccctggacaaactgttctttggatggaaagtctgtgcttgtgaatgggacgaaggagagggacgtggtctgcggaccatctccagccgacctctctccaggagcatcctctgcgaccccgcctgcccctgcgagagagccaggacactctccgcag

SEQ ID NO: 155 - protein, 396 aa, synthetic construct
  REGION 1..396: note = cyno CD137 ECD-mIgG2a AA
LQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 156 - DNA, 1188 bp, synthetic construct
  misc_feature 1..1188: note = cyno CD137 ECD-mIgG2a DNA
  source 1..1188: mol_type = other DNA
ctgcaggatctgtgtagtaactgcccagctggtacattctgtgataataacaggagtcagatttgcagtccctgtcctccaaatagtttctccagcgcaggtggacaaaggacctgtgacatatgcaggcagtgtaaaggtgttttcaagaccaggaaggagtgttcctccaccagcaatgcagagtgtgactgcatttcaggatatcactgcctgggggcagaatgcagcatgtgtgaacaggattgtaaacaaggtcaagaattgacaaaaaaaggttgtaaagactgttgctttgggacatttaatgaccagaaacgtggcatctgtcgaccctggacaaactgttctttggatggaaagtctgtgcttgtgaatgggacgaaggagagggacgtggtctgcggaccatctccagccgacctctctccaggagcatcctctgcgaccccgcctgcccctgcgagagagccaggacactctccgcaggagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 157 - protein, 277 aa, synthetic construct
  REGION 1..277: note = human CD40 AA
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 158 - DNA, 834 bp, synthetic construct
  misc_feature 1..834: note = human CD40 DNA
  source 1..834: mol_type = other DNA
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtga

SEQ ID NO: 159 - protein, 194 aa, synthetic construct
  REGION 1..194: note = huCD40 ECD AA (1-194)
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRA

SEQ ID NO: 160 - DNA, 582 bp, synthetic construct
  misc_feature 1..582: note = huCD40 ECD DNA (1-194)
  source 1..582: mol_type = other DNA
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagcc

SEQ ID NO: 161 - protein, 428 aa, synthetic construct
  REGION 1..428: note = human CD40 ECD-mIgG2a AA
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRASEPRGPTIKPCPPCKCPAPNLLGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 162 - DNA, 1284 bp, synthetic construct
  misc_feature 1..1284: note = human CD40 ECD-mIgG2a DNA
  source 1..1284: mol_type = other DNA
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgagttcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagctagcgagcccagagggcccacaatcaagccctgtcctccatgcaaatgcccagcacctaacctcttgggtggaccatccgtcttcatcttccctccaaagatcaaggatgtactcatgatctccctgagccccatggtcacatgtgtggtggtggatgtgagcgaggatgacccagatgtccagatcagctggttcgtgaacaacgtggaagtactcacagctcagacacaaacccatagagaggattacaacagtactctccgggtggtcagtgccctccccatccagcaccaggactggatgagtggcaaggagttcaaatgcaaggtcaacaacaaagccctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgctcagtggtccacgagggtctgcacaatcaccacacgactaagagcttctcccggactccgggtaaa

SEQ ID NO: 163 - protein, 277 aa, synthetic construct
  REGION 1..277: note = human OX40 AA
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 164 - DNA, 834 bp, synthetic construct
  misc_feature 1..834: note = human OX40 DNA
  source 1..834: mol_type = other DNA
atgtgcgtgggggctcggcggctgggccgcgggccgtgtgcggctctgctcctcctgggcctggggctgagcaccgtgacggggctccactgtgtcggggacacctaccccagcaacgaccggtgctgccacgagtgcaggccaggcaacgggatggtgagccgctgcagccgctcccagaacacggtgtgccgtccgtgcgggccgggcttctacaacgacgtggtcagctccaagccgtgcaagccctgcacgtggtgtaacctcagaagtgggagtgagcggaagcagctgtgcacggccacacaggacacagtctgccgctgccgggcgggcacccagcccctggacagctacaagcctggagttgactgtgccccctgccctccagggcacttctccccaggcgacaaccaggcctgcaagccctggaccaactgcaccttggctgggaagcacaccctgcagccggccagcaatagctcggacgcaatctgtgaggacagggaccccccagccacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctccggagggaccagaggctgccccccgatgcccacaagccccctgggggaggcagtttccggacccccatccaagaggagcaggccgacgcccactccaccctggccaagatctga

SEQ ID NO: 165 - protein, 216 aa, synthetic construct
  REGION 1..216: note = human OX40 ECD (1-216) AA
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVA

SEQ ID NO: 166 - DNA, 648 bp, synthetic construct
  misc_feature 1..648: note = human OX40 ECD (1-216) DNA
  source 1..648: mol_type = other DNA
atgtgcgtgggggctcggcggctgggccgcgggccgtgtgcggctctgctcctcctgggcctggggctgagcaccgtgacggggctccactgtgtcggggacacctaccccagcaacgaccggtgctgccacgagtgcaggccaggcaacgggatggtgagccgctgcagccgctcccagaacacggtgtgccgtccgtgcgggccgggcttctacaacgacgtggtcagctccaagccgtgcaagccctgcacgtggtgtaacctcagaagtgggagtgagcggaagcagctgtgcacggccacacaggacacagtctgccgctgccgggcgggcacccagcccctggacagctacaagcctggagttgactgtgccccctgccctccagggcacttctccccaggcgacaaccaggcctgcaagccctggaccaactgcaccttggctgggaagcacaccctgcagccggccagcaatagctcggacgcaatctgtgaggacagggaccccccagccacgcagccccaggagacccagggccccccggccaggcccatcactgtccagcccactgaagcctggcccagaacctcacagggaccctccacccggcccgtggaggtccccgggggccgtgcggttgcc

SEQ ID NO: 167 - protein, 222 aa, synthetic construct
  REGION 1..222: note = human OX40 ECD-His AA
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAHHHHHH

SEQ ID NO: 168 - DNA, 666 bp, synthetic construct
  misc_feature 1..666: note = human OX40 ECD-His DNA
  source 1..666: mol_type = other DNA
atgtgcgtgggggctcggcggctgggccgcgggccgtgtgcggctctgctcctcctgggcctggggctgagcaccgtgacggggctccactgtgtcggggacacctaccccagcaacgaccggtgctgccacgagtgcaggccaggcaacgggatggtgagccgctgcagccgctcccagaacacggtgtgccgtccgtgcgggccgggcttctacaacgacgtggtcagctccaagccgtgcaagccctgcacgtggtgtaacctcagaagtgggagtgagcggaagcagctgtgcacggccacacaggacacagtctgccgctgccgggcgggcacccagcccctggacagctacaagcctggagttgactgtgccccctgccctccagggcacttctccccaggcgacaaccaggcctgcaagccctggaccaactgcaccttggctgggaagcacaccctgcagccggccagcaatagctcggacgcaatctgtgaggacagggaccccccagccacgcagccccaggagacccagggccccccggccaggcccatcactgtccagcccactgaagcctggcccagaacctcacagggaccctccacccggcccgtggaggtccccgggggccgtgcggttgcccatcaccatcaccatcac

SEQ ID NO: 169 - protein, 98 aa, synthetic construct
  REGION 1..98: note = 3-23 germline AA
EVQLLESGGGLVQPGGSLRLSCAASGFTFSSYAISWVRQAPGKGLEWVSAISGSGGSTYYADSVKGRFTISRDNSKNTLYLQMNSLRAEDTAVYYCAR

SEQ ID NO: 170 - DNA, 294 bp, synthetic construct
  misc_feature 1..294: note = 3-23 germline DNA
  source 1..294: mol_type = other DNA
gaagtgcagctgctggaaagcggcggcggcctggtgcagccgggcggcagcctgcgcctgagctgcgcggcgagcggctttacctttagcagctatgcgattagctgggtgcgccaggcgccgggcaaaggcctggaatgggtgagcgcgattagcggcagcggcggtagcacctattatgcggatagcgtgaaaggccgctttaccattagccgcgataacagcaaaaacaccctgtatctgcagatgaacagcctgcgcgcggaagataccgccgtgtattattgcgcgcgt

SEQ ID NO: 171 - protein, 354 aa, synthetic construct
  REGION 1..354: note = BGA-7207 VH-Fc
EVQLLESGGGLVQPGGSLRLSCAASGFRLDTTEVGWVRQAPGKGLEWVSTIMGISGSTYYADSVKGRFTISRDNSKNTLYLQMNSLRAEDTAVYYCARVVDSLFDDSAVFDYWGQGTLVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - DNA, 1062 bp, synthetic construct
  misc_feature 1..1062: note = BGA-7207 VH-Fc DNA
  source 1..1062: mol_type = other DNA
gaggtccagttacttgagagtggtggaggtctggtccaaccaggaggttcgctgcgtttatcctgcgccgcgtctggattcagattggacaccaccgaagtaggttgggtgcgtcaagcgccggggaaaggactggaatgggtctccaccatcatgggtattagtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtcgattccctctttgatgacagcgccgtttttgattactggggacagggcaccttagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 173 - protein, 353 aa, synthetic construct
  REGION 1..353: note = BGA-4712 VH-Fc
EVQLLESGGGLVQPGGSLRLSCAASGFMLSAEDVGWVRQAPGKGLEWVSAILDFGGSTYYADSVKGRFTISRDNSKNTLYLQMNSLRAEDTAVYYCARVVYHAGGGVTFDYWGQGTLVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - DNA, 1059 bp, synthetic construct
  misc_feature 1..1059: note = BGA-4712 VH-Fc DNA
  source 1..1059: mol_type = other DNA
gaggtccagttacttgagagtggtggaggtctggtccaaccaggaggttcgctgcgtttatcctgcgccgcgtctggattcatgttgtccgccgaagacgtgggttgggtgcgtcaagcgccggggaaaggactggaatgggtctccgccatcttggattttggtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtctaccatgctggtggtggcgtcacctttgattactggggacagggcaccttagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 175 - protein, 226 aa, synthetic construct
  REGION 1..226: note = Inert Fc-A Fc-null version IgG1
DKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - DNA, 678 bp, synthetic construct
  misc_feature 1..678: note = Inert Fc-A Fc-null version IgG1 DNA
  source 1..678: mol_type = other DNA
gacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 177 - protein, 576 aa, synthetic construct
  REGION 1..576: note = A-CD137/CEA A-BGA-4712/CEA heavy chain AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFMLSAEDVGWVRQAPGKGLEWVSAILDFGGSTYYADSVKGRFTISRDNSKNTLYLQMNSLRAEDTAVYYCARVVYHAGGGVTFDYWGQGTLVTVSS

SEQ ID NO: 178 - DNA, 1728 bp, synthetic construct
  misc_feature 1..1728: note = A-CD137/CEA A-BGA-4712/CEA heavy chain DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcatgttgtccgccgaagacgtgggttgggtgcgtcaagcgccggggaaaggactggaatgggtctccgccatcttggattttggtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtctaccatgctggtggtggcgtcacctttgattactggggacagggcaccttagttacagtctcatcg

SEQ ID NO: 179 - protein, 214 aa, synthetic construct
  REGION 1..214: note = A-CD137/CEA anti-CEA Ab light chain AA
DIQMTQSPSSLSASVGDRVTITCRASENQYGYLAWYQQKPGKVPKLLIYNYKNLVEGVPSRFSGSGSGTDFTLTISSLQPEDVATYYCQHHLGTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - DNA, 642 bp, synthetic construct
  misc_feature 1..642: note = A-CD137/CEA anti-CEA Ab light chain DNA
  source 1..642: mol_type = other DNA
gatattcagatgacccagagcccgagcagcctgagcgcgagcgtgggcgatcgcgtgaccattacctgccgcgcgagcgaaaaccagtatggctatctggcgtggtatcagcagaaaccgggcaaagtgccgaaactgctgatttataactataaaaacctggtggaaggcgtgccgagccgctttagcggcagcggcagcggcaccgattttaccctgaccattagcagcctgcagccggaagatgtggcgacctattattgccagcatcatctgggcaccccgtatacctttggccagggcaccaaagtggaaattaaacgaacagtggcagccccttccgtcttcatttttcccccttctgacgaacagctgaaatcaggaactgctagcgtggtctgtctgctgaacaatttctaccccagagaggccaaggtgcagtggaaagtcgataacgctctgcagtccggcaattctcaggagagtgtgaccgaacaggactcaaaggatagcacatattccctgtctagtactctgaccctgagcaaagcagactacgagaagcacaaagtgtatgcctgtgaagtcacacaccaggggctgagttcaccagtcaccaagagtttcaacagaggggaatgc

SEQ ID NO: 181 - protein, 449 aa, synthetic construct
  REGION 1..449: note = B-CD137/CEA anti-CEA Ab heavy chain AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - DNA, 1347 bp, synthetic construct
  misc_feature 1..1347: note = B-CD137/CEA anti-CEA Ab heavy chain DNA
  source 1..1347: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 183 - protein, 341 aa, synthetic construct
  REGION 1..341: note = B-CD137/CEA B-BGA-4712/CEA light chain AA
DIQMTQSPSSLSASVGDRVTITCRASENQYGYLAWYQQKPGKVPKLLIYNYKNLVEGVPSRFSGSGSGTDFTLTISSLQPEDVATYYCQHHLGTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVQLLESGGGLVQPGGSLRLSCAASGFMLSAEDVGWVRQAPGKGLEWVSAILDFGGSTYYADSVKGRFTISRDNSKNTLYLQMNSLRAEDTAVYYCARVVYHAGGGVTFDYWGQGTLVTVSS

SEQ ID NO: 184 - DNA, 1023 bp, synthetic construct
  misc_feature 1..1023: note = B-CD137/CEA B-BGA-4712/CEA light chain DNA
  source 1..1023: mol_type = other DNA
gatattcagatgacccagagcccgagcagcctgagcgcgagcgtgggcgatcgcgtgaccattacctgccgcgcgagcgaaaaccagtatggctatctggcgtggtatcagcagaaaccgggcaaagtgccgaaactgctgatttataactataaaaacctggtggaaggcgtgccgagccgctttagcggcagcggcagcggcaccgattttaccctgaccattagcagcctgcagccggaagatgtggcgacctattattgccagcatcatctgggcaccccgtatacctttggccagggcaccaaagtggaaattaaacgaacagtggcagccccttccgtcttcatttttcccccttctgacgaacagctgaaatcaggaactgctagcgtggtctgtctgctgaacaatttctaccccagagaggccaaggtgcagtggaaagtcgataacgctctgcagtccggcaattctcaggagagtgtgaccgaacaggactcaaaggatagcacatattccctgtctagtactctgaccctgagcaaagcagactacgagaagcacaaagtgtatgcctgtgaagtcacacaccaggggctgagttcaccagtcaccaagagtttcaacagaggggaatgcgggggaggcgggtccgaggtccagttacttgagagtggtggaggtctggtccaaccaggaggttcgctgcgtttatcctgcgccgcgtctggattcatgttgtccgccgaagacgtgggttgggtgcgtcaagcgccggggaaaggactggaatgggtctccgccatcttggattttggtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtctaccatgctggtggtggcgtcacctttgattactggggacagggcaccttagttacagtctcatcg

SEQ ID NO: 185 - protein, 588 aa, synthetic construct
  REGION 1..588: note = C-CD137/CEA C-BGA-4712/CEA heavy chain AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSEVQLLESGGGLVQPGGSLRLSCAASGFMLSAEDVGWVRQAPGKGLEWVSAILDFGGSTYYADSVKGRFTISRDNSKNTLYLQMNSLRAEDTAVYYCARVVYHAGGGVTFDYWGQGTLVTVSSSGGGGSEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - DNA, 1764 bp, synthetic construct
  misc_feature 1..1764: note = C-CD137/CEA C-BGA-4712/CEA heavy chain DNA
  source 1..1764: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacgggggaggcgggtccgaggtccagttacttgagagtggtggaggtctggtccaaccaggaggttcgctgcgtttatcctgcgccgcgtctggattcatgttgtccgccgaagacgtgggttgggtgcgtcaagcgccggggaaaggactggaatgggtctccgccatcttggattttggtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtctaccatgctggtggtggcgtcacctttgattactggggacagggcaccttagttacagtctcatcgtctggtggaggcggttcagagcccaaatcttgtgacaaaactcacacatgcccaccgtgcccagcacctcctgctgccggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcgcagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 187 - protein, 577 aa, synthetic construct
  REGION 1..577: note = D-CD137/CEA D-BGA-4712/CEA heavy chain AA
EVQLLESGGGLVQPGGSLRLSCAASGFMLSAEDVGWVRQAPGKGLEWVSAILDFGGSTYYADSVKGRFTISRDNSKNTLYLQMNSLRAEDTAVYYCARVVYHAGGGVTFDYWGQGTLVTVSSSGGGGSQVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - DNA, 1731 bp, synthetic construct
  misc_feature 1..1731: note = D-CD137/CEA D-BGA-4712/CEA heavy chain DNA
  source 1..1731: mol_type = other DNA
gaggtccagttacttgagagtggtggaggtctggtccaaccaggaggttcgctgcgtttatcctgcgccgcgtctggattcatgttgtccgccgaagacgtgggttgggtgcgtcaagcgccggggaaaggactggaatgggtctccgccatcttggattttggtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtctaccatgctggtggtggcgtcacctttgattactggggacagggcaccttagttacagtctcatcgtccggtggaggcggttcacaggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 189 - protein, 353 aa, synthetic construct
  REGION 1..353: note = BGA-4712-M3-mutFc AA
EVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYWGQGTLVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - DNA, 1059 bp, synthetic construct
  misc_feature 1..1059: note = BGA-4712-M3-mutFc DNA
  source 1..1059: mol_type = other DNA
gaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattactggggacagggcaccttagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 191 - protein, 602 aa, synthetic construct
  REGION 1..602: note = A-CEAxCD137-M3 AA
GSATMDMRVPAQLLGLLLLWFPGSRSQVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYWGQGTLVTVSS

SEQ ID NO: 192 - DNA, 1821 bp, synthetic construct
  misc_feature 1..1821: note = A-CEAxCD137-M3 DNA
  source 1..1821: mol_type = other DNA
ggatccgccaccatggatatgagagttcctgctcaattgctggggttgctcttgctctggtttcctggctcgagaagccaggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattactggggacagggcaccttagttacagtctcatcgtgaaagcttcgtcag

SEQ ID NO: 193 - protein, 353 aa, synthetic construct
  REGION 1..353: note = BGA-7556-mutFc AA
EVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - DNA, 1059 bp, synthetic construct
  misc_feature 1..1059: note = BGA-7556-mutFc DNA
  source 1..1059: mol_type = other DNA
gaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattaccggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 195 - protein, 576 aa, synthetic construct
  REGION 1..576: note = Format A- BGA-7556 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYRGQGTQVTVSS

SEQ ID NO: 196 - DNA, 1728 bp, synthetic construct
  misc_feature 1..1728: note = Format A- BGA-7556 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattaccggggacagggcacccaagttacagtctcatcg

SEQ ID NO: 197 - protein, 107 aa, synthetic construct
  REGION 1..107: note = CH3 AA
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - DNA, 321 bp, synthetic construct
  misc_feature 1..321: note = CH3 DNA
  source 1..321: mol_type = other DNA
ggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 199 - protein, 234 aa, synthetic construct
  REGION 1..234: note = CH3- BGA-4712-M3 AA
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYWGQGTLVTVSS

SEQ ID NO: 200 - DNA, 702 bp, synthetic construct
  misc_feature 1..702: note = CH3- BGA-4712-M3 DNA
  source 1..702: mol_type = other DNA
ggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattactggggacagggcaccttagttacagtctcatcg

SEQ ID NO: 201 - protein, 122 aa, synthetic construct
  REGION 1..122: note = BGA-2690 AA
EVQLLESGGGLVQPGGSLRLSCAASGFTLSREDVGWVRQAPGKGLEWVSAILDFGSSTYYAESVKGRFTISRDNAKNTLYLQMSSLRAEDTAVYYCARVVYHAGGGVTYDYRGQGTQVTVSS

SEQ ID NO: 202 - DNA, 366 bp, synthetic construct
  misc_feature 1..366: note = BGA-2690 DNA
  source 1..366: mol_type = other DNA
gaggtccagttacttgagagtggtggaggtctggtccaaccaggaggttcgctgcgtttatcctgcgccgcgtctggattcacgctttccagagaagacgtgggttgggtgcgtcaagcgccggggaaaggactggaatgggtctccgccatcttggatttcggttcttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcg

SEQ ID NO: 203 - protein, 122 aa, synthetic construct
  REGION 1..122: note = BGA-3849 AA
EVQLLESGGGLVQPGGSLRLSCAASGFTLSHEDVGWVRQAPGKGLEWVSAILDSGGSTYYAESVKGRFTISRDNAKNTLYLQMSSLRAEDTAVYYCARYVYHAGGGVTFDTRGQGTQVTVSS

SEQ ID NO: 204 - DNA, 366 bp, synthetic construct
  misc_feature 1..366: note = BGA-3849 DNA
  source 1..366: mol_type = other DNA
gaggtccagttacttgagagtggtggaggtctggtccaaccaggaggttcgctgcgtttatcctgcgccgcgtctggattcacgctttcccatgaagacgtgggttgggtgcgtcaagcgccggggaaaggactggaatgggtctccgccatcttggattccggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatactcggggacagggcacccaagttacagtctcatcg

SEQ ID NO: 205 - protein, 122 aa, synthetic construct
  REGION 1..122: note = BGA-7916 AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHAGGGVTYDYRGQGTQVTVSS

SEQ ID NO: 206 - DNA, 366 bp, synthetic construct
  misc_feature 1..366: note = BGA-7916 DNA
  source 1..366: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcg

SEQ ID NO: 207 - protein, 122 aa, synthetic construct
  REGION 1..122: note = BGA-4988 AA
EVQLLESGGGLVQPGGSLRLSCAASGFTVSAEDVGWVRQAPGKGLEWVSAILDRGGSTYYAESVKGRFTISRDNAKNTLYLQMSSLRAEDTAVYYCARVVYHAGGGVTYDYRGQGTQVTVSS

SEQ ID NO: 208 - DNA, 366 bp, synthetic construct
  misc_feature 1..366: note = BGA-4988 DNA
  source 1..366: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggatcggggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcg

SEQ ID NO: 209 - protein, 122 aa, synthetic construct
  REGION 1..122: note = BGA-5164 AA
EVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVVYHAGGGVTYDYRGQGTQVTVSS

SEQ ID NO: 210 - DNA, 366 bp, synthetic construct
  misc_feature 1..366: note = BGA-5164 DNA
  source 1..366: mol_type = other DNA
gaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcg

SEQ ID NO: 211 - protein, 122 aa, synthetic construct
  REGION 1..122: note = BGA-3953 AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARIVYHAGGGVTFDTRGQGTQVTVSS

SEQ ID NO: 212 - DNA, 366 bp, synthetic construct
  misc_feature 1..366: note = BGA-3953 DNA
  source 1..366: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 213 - protein, 122 aa, synthetic construct
  REGION 1..122: note = BGA-5385 AA
EVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVIYHAGGGVTFDTRGQGTQVTVSS

SEQ ID NO: 214 - DNA, 366 bp, synthetic construct
  misc_feature 1..366: note = BGA-5385 DNA
  source 1..366: mol_type = other DNA
gaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttatctaccatgctggtggtggcgtcacctttgatactcggggacagggcacccaagttacagtctcatcg

SEQ ID NO: 215 - protein, 122 aa, synthetic construct
  REGION 1..122: note = BGA-9468 AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHSGGGVTFDLRGQGTQVTVSS

SEQ ID NO: 216 - DNA, 366 bp, synthetic construct
  misc_feature 1..366: note = BGA-9468 DNA
  source 1..366: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccattctggtggtggcgtcacctttgatcttcggggacagggcacccaagttacagtctcatcg

SEQ ID NO: 217 - protein, 122 aa, synthetic construct
  REGION 1..122: note = BGA-3285 AA
EVQLLESGGGLVQPGGSLRLSCAASGFTISAEDVGWVRQAPGKGLEWVSAILDFGGSTYYAESVKGRFTISRDNAKNTLYLQMSSLRAEDTAVYYCARVVYHAGGGVTFDTRGQGTQVTVSS

SEQ ID NO: 218 - DNA, 366 bp, synthetic construct
  misc_feature 1..366: note = BGA-3285 DNA
  source 1..366: mol_type = other DNA
gaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctttgatactcggggacagggcacccaagttacagtctcatcg

SEQ ID NO: 219 - protein, 122 aa, synthetic construct
  REGION 1..122: note = BGA-9442 AA
EVQLLESGGGLVQPGGSLRLSCAASGFTISAEDVGWVRQAPGKGLEWVSAILDSGGSTYYAESVKGRFTISRDNAKNTLYLQMSSLRAEDTAVYYCARYVYHAGGGVTFDLRGQGTQVTVSS

SEQ ID NO: 220 - DNA, 366 bp, synthetic construct
  misc_feature 1..366: note = BGA-9442 DNA
  source 1..366: mol_type = other DNA
gaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattctggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatcttcggggacagggcacccaagttacagtctcatcg

SEQ ID NO: 221 - protein, 122 aa, synthetic construct
  REGION 1..122: note = BGA-7746 AA
EVQLLESGGGLVQPGGSLRLSCAASGFTVSAEDVGWVRQAPGKGLEWVSAILDFGGSTYYAESVKGRFTISRDNAKNTLYLQMSSLRAEDTAVYYCARVVYHAGGGVTFDTRGQGTQVTVSS

SEQ ID NO: 222 - DNA, 366 bp, synthetic construct
  misc_feature 1..366: note = BGA-7746 DNA
  source 1..366: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattttggtggttcgacatactatgcagaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgatacgcggggacagggcacccaagttacagtctcatcg

SEQ ID NO: 223 - protein, 122 aa, synthetic construct
  REGION 1..122: note = BGA-8425 AA
EVQLLESGGGLVQPGGSLRLSCAASGFTLSAEDVGWVRQAPGKGLEWVSAILDSGGSTYYAESVKGRFTISRDNAKNTLYLQMSSLRAEDTAVYYCARVVYHAGGGVTYDYRGQGTQVTVSS

SEQ ID NO: 224 - DNA, 366 bp, synthetic construct
  misc_feature 1..366: note = BGA-8425 DNA
  source 1..366: mol_type = other DNA
gaggtccagttacttgagagtggtggaggtctggtccaaccaggaggttcgctgcgtttatcctgcgccgcgtctggattcacgctgtccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctatgattaccggggacagggcacccaagttacagtctcatcg

SEQ ID NO: 225 - protein, 122 aa, synthetic construct
  REGION 1..122: note = BGA-3386 AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTWGQGTLVTVSS

SEQ ID NO: 226 - DNA, 366 bp, synthetic construct
  misc_feature 1..366: note = BGA-3386 DNA
  source 1..366: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatacttggggacagggcaccttagttacagtctcatcg

SEQ ID NO: 227 - protein, 353 aa, synthetic construct
  REGION 1..353: note = BGA-5623-mutFc-AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - DNA, 1059 bp, synthetic construct
  misc_feature 1..1059: note = BGA-5623-mutFc DNA
  source 1..1059: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 229 - protein, 353 aa, synthetic construct
  REGION 1..353: note = BGA-2690- mutFc AA
EVQLLESGGGLVQPGGSLRLSCAASGFTLSREDVGWVRQAPGKGLEWVSAILDFGSSTYYAESVKGRFTISRDNAKNTLYLQMSSLRAEDTAVYYCARVVYHAGGGVTYDYRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - DNA, 1059 bp, synthetic construct
  misc_feature 1..1059: note = BGA-2690 mutFc DNA
  source 1..1059: mol_type = other DNA
gaggtccagttacttgagagtggtggaggtctggtccaaccaggaggttcgctgcgtttatcctgcgccgcgtctggattcacgctttccagagaagacgtgggttgggtgcgtcaagcgccggggaaaggactggaatgggtctccgccatcttggatttcggttcttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 231 - protein, 353 aa, synthetic construct
  REGION 1..353: note = BGA-3849-mutFc AA
EVQLLESGGGLVQPGGSLRLSCAASGFTLSHEDVGWVRQAPGKGLEWVSAILDSGGSTYYAESVKGRFTISRDNAKNTLYLQMSSLRAEDTAVYYCARYVYHAGGGVTFDTRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - DNA, 1059 bp, synthetic construct
  misc_feature 1..1059: note = BGA-3849 mutFc DNA
  source 1..1059: mol_type = other DNA
gaggtccagttacttgagagtggtggaggtctggtccaaccaggaggttcgctgcgtttatcctgcgccgcgtctggattcacgctttcccatgaagacgtgggttgggtgcgtcaagcgccggggaaaggactggaatgggtctccgccatcttggattccggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatactcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 233 - protein, 353 aa, synthetic construct
  REGION 1..353: note = BGA-7916-mutFc AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHAGGGVTYDTRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - DNA, 1059 bp, synthetic construct
  misc_feature 1..1059: note = BGA-7916-mutFc DNA
  source 1..1059: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 235 - protein, 353 aa, synthetic construct
  REGION 1..353: note = BGA-4988-mutFc AA
EVQLLESGGGLVQPGGSLRLSCAASGFTVSAEDVGWVRQAPGKGLEWVSAILDRGGSTYYAESVKGRFTISRDNAKNTLYLQMSSLRAEDTAVYYCARVVYHAGGGVTYDTRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - DNA, 1059 bp, synthetic construct
  misc_feature 1..1059: note = BGA-4988-mutFc DNA
  source 1..1059: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggatcggggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 237 - protein, 353 aa, synthetic construct
  REGION 1..353: note = BGA-5164-mutFc AA
EVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVVYHAGGGVTYDYRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - DNA, 1059 bp, synthetic construct
  misc_feature 1..1059: note = BGA-5164-mutFc DNA
  source 1..1059: mol_type = other DNA
gaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 239 - protein, 353 aa, synthetic construct
  REGION 1..353: note = BGA-3953-mutFc AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARIVYHAGGGVTFDTRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - DNA, 1059 bp, synthetic construct
  misc_feature 1..1059: note = BGA-3953-mutFc DNA
  source 1..1059: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 241 - protein, 353 aa, synthetic construct
  REGION 1..353: note = BGA-5385-mutFc AA
EVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVIYHAGGGVTYDYRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - DNA, 1059 bp, synthetic construct
  misc_feature 1..1059: note = BGA-5385-mutFc DNA
  source 1..1059: mol_type = other DNA
gaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttatctaccatgctggtggtggcgtcacctatgattatcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 243 - protein, 353 aa, synthetic construct
  REGION 1..353: note = BGA-9468-mutFc AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHSGGGVTFDLRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - DNA, 1059 bp, synthetic construct
  misc_feature 1..1059: note = BGA-9468-mutFc DNA
  source 1..1059: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccattctggtggtggcgtcacctttgatcttcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 245 - protein, 353 aa, synthetic construct
  REGION 1..353: note = BGA-3285-mutFc AA
EVQLLESGGGLVQPGGSLRLSCAASGFTISAEDVGWVRQAPGKGLEWVSAILDFGGSTYYAESVKGRFTISRDNAKNTLYLQMSSLRAEDTAVYYCARVVYHAGGGVTFDTRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 246 - DNA, 1059 bp, synthetic construct
  misc_feature 1..1059: note = BGA-3285-mutFc DNA
  source 1..1059: mol_type = other DNA
gaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctttgatactcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 247 - protein, 353 aa, synthetic construct
  REGION 1..353: note = BGA-9442-mutFc AA
EVQLLESGGGLVQPGGSLRLSCAASGFTISAEDVGWVRQAPGKGLEWVSAILDSGGSTYYAESVKGRFTISRDNAKNTLYLQMSSLRAEDTAVYYCARYVYHAGGGVTFDLRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 248 - DNA, 1059 bp, synthetic construct
  misc_feature 1..1059: note = BGA-9442-mutFc DNA
  source 1..1059: mol_type = other DNA
gaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattctggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatcttcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 249 - protein, 353 aa, synthetic construct
  REGION 1..353: note = BGA-7746-mutFc AA
EVQLLESGGGLVQPGGSLRLSCAASGFTVSAEDVGWVRQAPGKGLEWVSAILDFGGSTYYAESVKGRFTISRDNAKNTLYLQMSSLRAEDTAVYYCARVVYHAGGGVTFDTRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - DNA, 1059 bp, synthetic construct
  misc_feature 1..1059: note = BGA-7746-mutFc DNA
  source 1..1059: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattttggtggttcgacatactatgcagaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgatacgcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 251 - protein, 353 aa, synthetic construct
  REGION 1..353: note = BGA-8425-mutFc AA
EVQLLESGGGLVQPGGSLRLSCAASGFTLSAEDVGWVRQAPGKGLEWVSAILDSGGSTYYAESVKGRFTISRDNAKNTLYLQMSSLRAEDTAVYYCARVVYHAGGGVTYDYRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 252 - DNA, 1059 bp, synthetic construct
  misc_feature 1..1059: note = BGA-8425-mutFc DNA
  source 1..1059: mol_type = other DNA
gaggtccagttacttgagagtggtggaggtctggtccaaccaggaggttcgctgcgtttatcctgcgccgcgtctggattcacgctgtccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctatgattaccggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 253 - protein, 353 aa, synthetic construct
  REGION 1..353: note = BGA-3386-mutFc AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTWGQGTLVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 254 - DNA, 1059 bp, synthetic construct
  misc_feature 1..1059: note = BGA-3386-mutFc DNA
  source 1..1059: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatacttggggacagggcaccttagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 255 - protein, 576 aa, synthetic construct
  REGION 1..576: note = Format A BGA-5623AA (BE-189)
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSS

SEQ ID NO: 256 - DNA, 1728 bp, synthetic construct
  misc_feature 1..1728: note = Format A BGA-5623 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 257 - protein, 576 aa, synthetic construct
  REGION 1..576: note = Format A BGA-2690 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSREDVGWVRQAPGKGLEWVSAILDFGSSTYYAESVKGRFTISRDNAKNTLYLQMSSLRAEDTAVYYCARVVYHAGGGVTYDYRGQGTQVTVSS

SEQ ID NO: 258 - DNA, 1728 bp, synthetic construct
  misc_feature 1..1728: note = Format A BGA-2690 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgctttccagagaagacgtgggttgggtgcgtcaagcgccggggaaaggactggaatgggtctccgccatcttggatttcggttcttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcg

SEQ ID NO: 259 - protein, 576 aa, synthetic construct
  REGION 1..576: note = Format A BGA-3849 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSHEDVGWVRQAPGKGLEWVSAILDSGGSTYYAESVKGRFTISRDNAKNTLYLQMSSLRAEDTAVYYCARYVYHAGGGVTFDTRGQGTQVTVSS

SEQ ID NO: 260 - DNA, 1728 bp, synthetic construct
  misc_feature 1..1728: note = Format A BGA-3849 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgctttcccatgaagacgtgggttgggtgcgtcaagcgccggggaaaggactggaatgggtctccgccatcttggattccggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatactcggggacagggcacccaagttacagtctcatcg

SEQ ID NO: 261 - protein, 576 aa, synthetic construct
  REGION 1..576: note = Format A BGA-7916 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHAGGGVTYDYRGQGTQVTVSS

SEQ ID NO: 262 - DNA, 1728 bp, synthetic construct
  misc_feature 1..1728: note = Format A BGA-7916 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcg

SEQ ID NO: 263 - protein, 576 aa, synthetic construct
  REGION 1..576: note = Format A BGA-4988 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDRGGSTYYAESVKGRFTISRDNAKNTLYLQMSSLRAEDTAVYYCARVVYHAGGGVTYDYRGQGTQVTVSS

SEQ ID NO: 264 - DNA, 1728 bp, synthetic construct
  misc_feature 1..1728: note = Format A BGA-4988 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggatcggggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcg

SEQ ID NO: 265 - protein, 576 aa, synthetic construct
  REGION 1..576: note = Format A BGA-5164 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVVYHAGGGVTYDYRGQGTQVTVSS

SEQ ID NO: 266 - DNA, 1728 bp, synthetic construct
  misc_feature 1..1728: note = Format A BGA-5164 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcg

SEQ ID NO: 267 - protein, 576 aa, synthetic construct
  REGION 1..576: note = Format A BGA-3953 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARIVYHAGGGVTFDTRGQGTQVTVSS

SEQ ID NO: 268 - DNA, 1728 bp, synthetic construct
  misc_feature 1..1728: note = Format A BGA-3953 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 269 - protein, 576 aa, synthetic construct
  REGION 1..576: note = Format A BGA-5385 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVIYHAGGGVTFDTRGQGTQVTVSS

SEQ ID NO: 270 - DNA, 1728 bp, synthetic construct
  misc_feature 1..1728: note = Format A BGA-5385 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttatctaccatgctggtggtggcgtcacctttgatactcggggacagggcacccaagttacagtctcatcg

SEQ ID NO: 271 - protein, 576 aa, synthetic construct
  REGION 1..576: note = Format A BGA-9468 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHSGGGVTFDLRGQGTQVTVSS

SEQ ID NO: 272 - DNA, 1728 bp, synthetic construct
  misc_feature 1..1728: note = Format A BGA-9468 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccattctggtggtggcgtcacctttgatcttcggggacagggcacccaagttacagtctcatcg

SEQ ID NO: 273 - protein, 576 aa, synthetic construct
  REGION 1..576: note = Format A BGA-3285 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTISAEDVGWVRQAPGKGLEWVSAILDFGGSTYYAESVKGRFTISRDNAKNTLYLQMSSLRAEDTAVYYCARVVYHAGGGVTFDTRGQGTQVTVSS

SEQ ID NO: 274 - DNA, 1728 bp, synthetic construct
  misc_feature 1..1728: note = Format A BGA-3285 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctttgatactcggggacagggcacccaagttacagtctcatcg

SEQ ID NO: 275 - protein, 576 aa, synthetic construct
  REGION 1..576: note = Format A BGA-9442 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTISAEDVGWVRQAPGKGLEWVSAILDSGGSTYYAESVKGRFTISRDNAKNTLYLQMSSLRAEDTAVYYCARYVYHAGGGVTFDLRGQGTQVTVSS

SEQ ID NO: 276 - DNA, 1728 bp, synthetic construct
  misc_feature 1..1728: note = Format A BGA-9442 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattctggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatcttcggggacagggcacccaagttacagtctcatcg

SEQ ID NO: 277 - protein, 576 aa, synthetic construct
  REGION 1..576: note = Format A BGA-7746 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDFGGSTYYAESVKGRFTISRDNAKNTLYLQMSSLRAEDTAVYYCARVVYHAGGGVTFDTRGQGTQVTVSS

SEQ ID NO: 278 - DNA, 1728 bp, synthetic construct
  misc_feature 1..1728: note = Format A BGA-7746 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattttggtggttcgacatactatgcagaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgatacgcggggacagggcacccaagttacagtctcatcg

SEQ ID NO: 279 - protein, 576 aa, synthetic construct
  REGION 1..576: note = Format A BGA-8425 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSAEDVGWVRQAPGKGLEWVSAILDSGGSTYYAESVKGRFTISRDNAKNTLYLQMSSLRAEDTAVYYCARVVYHAGGGVTYDYRGQGTQVTVSS

SEQ ID NO: 280 - DNA, 1728 bp, synthetic construct
  misc_feature 1..1728: note = Format A BGA-8425 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgctgtccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctatgattaccggggacagggcacccaagttacagtctcatcg

SEQ ID NO: 281 - protein, 576 aa, synthetic construct
  REGION 1..576: note = Format A BGA-3386 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTWGQGTLVTVSS

SEQ ID NO: 282 - DNA, 1728 bp, synthetic construct
  misc_feature 1..1728: note = Format A BGA-3386 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatacttggggacagggcaccttagttacagtctcatcg

SEQ ID NO: 283 - protein, 354 aa, synthetic construct
  REGION 1..354: note = BGA-5623 IgG1 AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 284 - DNA, 1062 bp, synthetic construct
  misc_feature 1..1062: note = BGA-5623 IgG1 DNA
  source 1..1062: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcggagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 285 - protein, 350 aa, synthetic construct
  REGION 1..350: note = BGA-5623 IgG4 AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 286 - DNA, 1050 bp, synthetic construct
  misc_feature 1..1050: note = BGA-5623 IgG4 DNA
  source 1..1050: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcggaatccaagtatggtccgccatgtccaccgtgcccggcacccgagtttttgggagggcctagtgtgtttttgtttcctcctaagccaaaggacacgcttatgattagtcgcacacccgaggttacctgtgtagtggtggacgtatcacaagaggatccggaagtccaatttaattggtacgtcgacggagtggaagtccacaatgcaaagaccaagcctagggaagaacagttcaattctacctatcgctggtctgtactcactgtactccaccaggattggctcaatggaaaggaatacaaatgcaaggtttccaacaaagggctcccctcttccattgagaaaactattagcaaggctaaaggccaacccagagaaccacaggtttatacccttccaccttcccaggaggagatgacgaagaatcaggttagtcttacgtgcctcgttaaaggattctacccttcagatatcgcagttgaatgggagtctaacgggcaacccgagaacaactacaagaccaccccgccggtgctggactcagatggtagtttctttctttacagtcggcttaccgtcgacaagtcacgctggcaggagggaaatgtattctcctgcagcgtaatgcacgaagctctgcacaatcactatacccagaagtcactgagcctttctctgggtaag

SEQ ID NO: 287 - protein, 447 aa, synthetic construct
  REGION 1..447: note = Urelumab analog-Heavy chain AA
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 288 - DNA, 1341 bp, synthetic construct
  misc_feature 1..1341: note = Urelumab analog-Heavy chain DNA
  source 1..1341: mol_type = other DNA
caagttcaacttcagcagtggggagcgggtctcttgaagcctagtgagacgctgagcctgacctgtgcagtctacgggggttcattttctgggtactactggtcctggatacgccaaagcccagagaaaggcctggaatggattggggagataaatcacgggggttatgtgacatataatccttctcttgagagccgggtcacgatatctgttgacacaagtaaaaatcagttctccctcaaactgagctccgttaccgcagcagacactgccgtttactactgtgcgcgggactatgggccaggtaattacgattggtacttcgacctgtggggtagaggaacgctggtcactgtaagcagtgcatctacaaagggccccagcgtgtttccccttgcaccatgttcacggagcactagtgaaagcactgccgcgctcgggtgtctcgttaaggattattttccggagcctgtcactgtctcttggaactctggtgcactgacatcaggcgttcacacgttccctgcagtactccagagctcaggattgtacagcctgtccagtgtagttacagtgccttcctcctcccttggaacaaagacctacacctgcaacgtggaccataagcccagcaatacgaaagtagacaaacgagtcgaatccaagtatggtccgccatgtccaccgtgcccggcacccgagtttttgggagggcctagtgtgtttttgtttcctcctaagccaaaggacacgcttatgattagtcgcacacccgaggttacctgtgtagtggtggacgtatcacaagaggatccggaagtccaatttaattggtacgtcgacggagtggaagtccacaatgcaaagaccaagcctagggaagaacagttcaattctacctatcgctggtctgtactcactgtactccaccaggattggctcaatggaaaggaatacaaatgcaaggtttccaacaaagggctcccctcttccattgagaaaactattagcaaggctaaaggccaacccagagaaccacaggtttatacccttccaccttcccaggaggagatgacgaagaatcaggttagtcttacgtgcctcgttaaaggattctacccttcagatatcgcagttgaatgggagtctaacgggcaacccgagaacaactacaagaccaccccgccggtgctggactcagatggtagtttctttctttacagtcggcttaccgtcgacaagtcacgctggcaggagggaaatgtattctcctgcagcgtaatgcacgaagctctgcacaatcactatacccagaagtcactgagcctttctctgggtaag

SEQ ID NO: 289 - protein, 216 aa, synthetic construct
  REGION 1..216: note = Urelumab analog-Light chain AA
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 290 - DNA, 648 bp, synthetic construct
  misc_feature 1..648: note = Urelumab analog-Light chain DNA
  source 1..648: mol_type = other DNA
gaaatcgtgctgacccaatcacctgcgaccctctcccttagccctggagaacgagcaactctcagttgcagagcaagccagagcgtcagctcatatctcgcatggtatcaacaaaaaccaggccaagctcctagactgttgatttatgatgcctctaaccgggcgactggcataccagcccgctttagcgggtctgggagtggcactgattttacgctcacgatttcatctttggagccagaagatttcgctgtatactactgtcaacaaaggtcaaactggccgcctgcacttacgttcggcgggggtaccaaggtggagattaagaggacagttgcggcgccatcagtttttatattcccaccctccgatgagcagttgaaaagtggcacggcatcagttgtctgtctgctgaacaatttctatccacgggaggcgaaagtccaatggaaagtggacaatgcgcttcagagcgggaactctcaggagagtgtcactgaacaggattcaaaggactccacgtactcactctcatccactctcaccctttcaaaggcggattacgaaaagcataaggtctacgcttgtgaggtaacacatcaggggctttccagtcctgtaaccaagagcttcaatcgcggcgaatgc

SEQ ID NO: 291 - protein, 442 aa, synthetic construct
  REGION 1..442: note = Utomilumab analog-Heavy chain AA
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 292 - DNA, 1326 bp, synthetic construct
  misc_feature 1..1326: note = Utomilumab analog-Heavy chain DNA
  source 1..1326: mol_type = other DNA
gaagttcaactggtacagtccggtgctgaagttaaaaaacccggagagagtctgagaatctcttgcaaggggtctgggtactcttttagtacatattggatatcttgggtgagacaaatgccggggaaaggcctcgaatggatgggcaagatttatcccggagacagctatacaaattactcacctagttttcaaggtcaggtaactatcagcgcggacaaatccataagtacggcctatctgcaatggtcttccttgaaggcatccgacacggcgatgtattactgcgcaagagggtatggtatattcgattactgggggcagggcactctcgttaccgtgagcagcgcttcaacaaaaggcccatctgtcttccctctcgcgccttgctcccgctctacttcagagtcaacagctgctctggggtgcttggtgaaggattactttcctgaaccggttactgtcagctggaattctggggctttgacgagcggagttcacactttcccagctgtactgcaaagttctggactttacagcttgagctccgtggtgacagtgccgagctcaaacttcggtacgcagacatatacatgcaatgtggatcataaacccagcaatacgaaagtagataagacagtcgaaaggaagtgttgtgtagagtgcccaccgtgccccgcccctccggttgcaggcccatcagtgtttctcttcccgcccaaacctaaggacacgctgatgatttcacgaacgcccgaggtcacctgtgtcgtagtcgatgtgtcccatgaggacccagaagttcagttcaattggtatgtagacggcgtggaggtacacaatgcaaaaacgaaaccacgcgaggaacagttcaatagtacgttccgggtcgtgagtgtgctcacggtggttcaccaagactggctgaacgggaaagaatataagtgcaaagtatccaataaaggcctgcccgcacccatcgaaaaaactataagtaaaactaaagggcaaccgcgagagcctcaggtctatactttgccaccgtcacgcgaagagatgaccaagaaccaagtaagtcttacttgcttggtcaaggggttctacccctcagatatagcggtggagtgggaatccaatggccaacccgagaacaattacaagacaacaccaccaatgctggactctgacgggagtttctttctgtatagtaaacttaccgtcgataaatcacgctggcagcaaggcaacgtattctcatgctcagtaatgcatgaagccttgcataatcactacactcagaaaagcctgtccctctctccaggtaag

SEQ ID NO: 293 - protein, 214 aa, synthetic construct
  REGION 1..214: note = Utomilumab analog-Light chain AA
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 294 - DNA, 642 bp, synthetic construct
  misc_feature 1..642: note = Utomilumab analog-Light chain DNA
  source 1..642: mol_type = other DNA
agctacgagttgactcagccaccatccgtcagtgttagccccggacagacggcgtcaattacttgtagtggagacaacattggcgatcagtacgcccattggtatcagcaaaaaccgggtcaatcaccagttctggtgatatatcaggataagaatcgaccatcagggattccagagaggttctcaggtagtaactcaggtaacacagctactctgaccataagtggtacccaggctatggacgaagcagactactactgcgccacctacacaggcttcggatcacttgcggttttcgggggcggaaccaaactaaccgttcttgggcaacctaaggctgccccgtccgtgactctttttcctccgtcttccgaagagcttcaagccaataaagcaaccttggtatgtttgatttccgatttttaccctggagcggtgacggtggcatggaaagctgattcaagccctgtgaaagctggggtggaaactactacgccgtccaaacagagcaataataagtacgcggcctccagctatttgtcacttaccccggagcaatggaagtcacaccgaagttacagttgtcaagttactcacgaaggcagcactgttgaaaagaccgtagcacccaccgaatgctca

SEQ ID NO: 295 - protein, 703 aa, synthetic construct
  REGION 1..703: note = BE-718 A-BGA-5623- BGA-5623 heavy chain AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSS

SEQ ID NO: 296 - DNA, 2109 bp, synthetic construct
  misc_feature 1..2109: note = BE-718 A-BGA-5623- BGA-5623 heavy chain DNA
  source 1..2109: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcgggcggcggaggatcggaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 297 - protein, 577 aa, synthetic construct
  REGION 1..577: note = BE-740 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSS

SEQ ID NO: 298 - DNA, 1731 bp, synthetic construct
  misc_feature 1..1731: note = BE-740 DNA
  source 1..1731: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 299 - protein, 450 aa, synthetic construct
  REGION 1..450: note = BE-942 (ZW1+1) anti-CEA heavy chain AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAWLGCEVTDYFPEPVTVSWNSGALTSGVHTFPAVLESSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 300 - DNA, 1350 bp, synthetic construct
  misc_feature 1..1350: note = BE-942 (ZW1+1) anti-CEA heavy chain DNA
  source 1..1350: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaagggaccaagcgtgttcccactggcacccagctccaagagcacctccggaggaacagcatggctgggatgcgaggtgaccgactacttccccgagcctgtgacagtgtcttggaacagcggcgccctgaccagcggagtgcacacatttcctgccgtgctggagtctagcggcctgtactccctgtcctctgtggtgaccgtgcctagctcctctctgggcacccagacatatatctgtaacgtgaatcacaagccatctaatacaaaggtggataagaaggtggagccaaagtcctgcgacaagacccacacatgcccaccttgtccagcaccagaggcagcaggaggaccatccgtgttcctgtttccacccaagcccaaggataccctgatgatctctcggacccccgaggtgacatgcgtggtggtgagcgtgtcccacgaggaccctgaggtgaagttcaactggtacgtggatggcgtggaggtgcacaatgccaagacaaagccccgggaggagcagtacaacagcacctatagagtggtgtccgtgctgacagtgctgcaccaggactggctgaacggcaaggagtataagtgcaaggtgtctaataaggccctgcctgccccaatcgagaagaccatcagcaaggcaaagggacagcccagggagcctcaggtgtacgtgctgcctccaagccgcgacgagctgacaaagaaccaggtgtccctgctgtgcctggtgaagggcttctatccttccgatatcgccgtggagtgggagtctaatggccagccagagaacaattacctgacctggccccctgtgctggactccgatggctctttctttctgtattctaagctgacagtggataagagcaggtggcagcagggcaacgtgttttcttgcagcgtgatgcacgaggccctgcacaatcactacacccagaagtccctgtctctgagccctggcaag

SEQ ID NO: 301 - protein, 214 aa, synthetic construct
  REGION 1..214: note = BE-942 (ZW1+1) anti-CEA light chain AA
DIQMTQSPSSLSASVGDRVTITCRASENQYGYLAWYQQKPGKVPKLLIYNYKNLVEGVPSRFSGSGSGTDFTLTISSLQPEDVATYYCQHHLGTPYTFGQGTKVEIKRTVAAPSVAIFPPSDERLKSGTASVVCVLNNFYPREAKVQWKVDNALQSGNSQESVTEQDSKDSTYSLSSRLTLSKADYEKHKVYACEVTHQGLSSPVTKSFNRGEC

SEQ ID NO: 302 - DNA, 642 bp, synthetic construct
  misc_feature 1..642: note = BE-942 (ZW1+1) anti-CEA light chain DNA
  source 1..642: mol_type = other DNA
gatattcagatgacccagagcccgagcagcctgagcgcgagcgtgggcgatcgcgtgaccattacctgccgcgcgagcgaaaaccagtatggctatctggcgtggtatcagcagaaaccgggcaaagtgccgaaactgctgatttataactataaaaacctggtggaaggcgtgccgagccgctttagcggcagcggcagcggcaccgattttaccctgaccattagcagcctgcagccggaagatgtggcgacctattattgccagcatcatctgggcaccccgtatacctttggccagggcaccaaagtggaaattaaaaggacagtggcagcaccaagcgtggcaatcttcccaccttccgacgagagactgaagtccggcaccgctagcgtggtgtgcgtgctgaacaacttctacccccgggaggccaaggtgcagtggaaggtggataacgccctgcagtccggcaattctcaggagagcgtgaccgagcaggactccaaggattctacatatagcctgagctccaggctgaccctgtctaaggccgactacgagaagcacaaggtgtatgcctgcgaggtgacacaccagggcctgtctagccccgtgaccaagtccttcaaccgcggcgagtgt

SEQ ID NO: 303 - protein, 354 aa, synthetic construct
  REGION 1..354: note = BE-942 (ZW1+1) anti-CD137 heavy chain AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 304 - DNA, 1062 bp, synthetic construct
  misc_feature 1..1062: note = BE-942 (ZW1+1) anti-CD137 heavy chain DNA
  source 1..1062: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcggagccaaagtcctccgacaagacccacacatgcccaccttgtccagcaccagaggcagcaggaggaccatccgtgttcctgtttccacccaagcccaaggataccctgatgatctctcggacccccgaggtgacatgcgtggtggtgagcgtgtcccacgaggaccctgaggtgaagttcaactggtacgtggatggcgtggaggtgcacaatgccaagacaaagccccgggaggagcagtacaacagcacctatagagtggtgtccgtgctgacagtgctgcaccaggactggctgaacggcaaggagtataagtgcaaggtgtctaataaggccctgcctgccccaatcgagaagaccatcagcaaggcaaagggacagcccagggagcctcaggtgtacgtgtatcctccaagccgcgacgagctgaccaagaaccaggtgtccctgacatgtctggtgaagggcttttacccttccgatatcgccgtggagtgggagtctaatggccagccagagaacaattataagaccacaccccctgtgctggactccgatggctctttcgccctggtgtctaagctgaccgtggataagagcaggtggcagcagggcaacgtgttttcttgcagcgtgatgcacgaggccctgcacaatcactacacacagaagtccctgtctctgagccctggcaag

SEQ ID NO: 305 - protein, 481 aa, synthetic construct
  REGION 1..481: note = BE-755 (ZW1+2) anti-CD137 heavy chain AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 306 - DNA, 1443 bp, synthetic construct
  misc_feature 1..1443: note = BE-755 (ZW1+2) anti-CD137 heavy chain DNA
  source 1..1443: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcgggcggcggagggagcgaggtgcagctgcttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagtgaccgtgtccagcgagccaaagtcctccgacaagacccacacatgcccaccttgtccagcaccagaggcagcaggaggaccatccgtgttcctgtttccacccaagcccaaggataccctgatgatctctcggacccccgaggtgacatgcgtggtggtgagcgtgtcccacgaggaccctgaggtgaagttcaactggtacgtggatggcgtggaggtgcacaatgccaagacaaagccccgggaggagcagtacaacagcacctatagagtggtgtccgtgctgacagtgctgcaccaggactggctgaacggcaaggagtataagtgcaaggtgtctaataaggccctgcctgccccaatcgagaagaccatcagcaaggcaaagggacagcccagggagcctcaggtgtacgtgtatcctccaagccgcgacgagctgaccaagaaccaggtgtccctgacatgtctggtgaagggcttttacccttccgatatcgccgtggagtgggagtctaatggccagccagagaacaattataagaccacaccccctgtgctggactccgatggctctttcgccctggtgtctaagctgaccgtggataagagcaggtggcagcagggcaacgtgttttcttgcagcgtgatgcacgaggccctgcacaatcactacacacagaagtccctgtctctgagccctggcaag

SEQ ID NO: 307 - protein, 582 aa, synthetic construct
  REGION 1..582: note = BE-562 (E-mutFc) heavy chain AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSEPKSSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSQVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 308 - DNA, 1746 bp, synthetic construct
  misc_feature 1..1746: note = BE-562 (E-mutFc) heavy chain DNA
  source 1..1746: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcggagccaaagtcctctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcacaggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgac

SEQ ID NO: 309 - protein, 583 aa, synthetic construct
  REGION 1..583: note = BE-375 (E-wtIgG1 Fc) heavy chain AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSQVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 310 - DNA, 1749 bp, synthetic construct
  misc_feature 1..1749: note = BE-375 (E-wtIgG1 Fc) heavy chain DNA
  source 1..1749: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcggagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggaggcggttcacaggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgac

SEQ ID NO: 311 - protein, 586 aa, synthetic construct
  REGION 1..586: note = BE-244 (A-(G4S)3) heavy chain AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSS

SEQ ID NO: 312 - DNA, 1758 bp, synthetic construct
  misc_feature 1..1758: note = BE-244 (A-(G4S)3) heavy chain DNA
  source 1..1758: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcaggcggcggaggatctggtggcggtgggtccgaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 313 - protein, 576 aa, synthetic construct; identical to SEQ ID NO: 255
  REGION 1..576: note = BE-146 heavy chain AA

SEQ ID NO: 314 - DNA, 1728 bp, synthetic construct; identical to SEQ ID NO: 256
  misc_feature 1..1728: note = BE-146 heavy chain DNA
  source 1..1728: mol_type = other DNA

SEQ ID NO: 315 - protein, 118 aa, synthetic construct
  REGION 1..118: note = Tislelizumab VH
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 316 - protein, 107 aa, synthetic construct
  REGION 1..107: note = Tislelizumab VL
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 317-321 - skipped (5 entries)

SEQ ID NO: 322 - protein, 4 aa, synthetic construct
  REGION 1..4: note = linker
GGGS

SEQ ID NO: 323 - protein, 4 aa, synthetic construct
  REGION 1..4: note = linker
SGGG

SEQ ID NO: 324 - protein, 5 aa, synthetic construct
  REGION 1..5: note = linker
GGGGS

SEQ ID NO: 325 - protein, 7 aa, synthetic construct
  REGION 1..7: note = linker
GGGGSGS

SEQ ID NO: 326 - protein, 7 aa, synthetic construct
  REGION 1..7: note = linker
GGGGSGS

SEQ ID NO: 327 - protein, 8 aa, synthetic construct
  REGION 1..8: note = linker
GGGGSGGS

SEQ ID NO: 328 - protein, 10 aa, synthetic construct
  REGION 1..10: note = linker
GGGGSGGGGS

SEQ ID NO: 329 - protein, 15 aa, synthetic construct
  REGION 1..15: note = linker
GGGGSGGGGSGGGGS

SEQ ID NO: 330 - protein, 16 aa, synthetic construct
  REGION 1..16: note = linker
AKTTPKLEEGEFSEAR

SEQ ID NO: 331 - protein, 17 aa, synthetic construct
  REGION 1..17: note = linker
AKTTPKLEEGEFSEARV

SEQ ID NO: 332 - protein, 9 aa, synthetic construct
  REGION 1..9: note = linker
AKTTPKLGG

SEQ ID NO: 333 - protein, 10 aa, synthetic construct
  REGION 1..10: note = linker
SAKTTPKLGG

SEQ ID NO: 334 - protein, 17 aa, synthetic construct
  REGION 1..17: note = linker
AKTTPKLEEGEFSEARV

SEQ ID NO: 335 - protein, 6 aa, synthetic construct
  REGION 1..6: note = linker
SAKTTP

SEQ ID NO: 336 - protein, 10 aa, synthetic construct
  REGION 1..10: note = linker
SAKTTPKLGG

SEQ ID NO: 337 - protein, 6 aa, synthetic construct
  REGION 1..6: note = linker
RADAAP

SEQ ID NO: 338 - protein, 9 aa, synthetic construct
  REGION 1..9: note = limker
RADAAPTVS

SEQ ID NO: 339 - protein, 12 aa, synthetic construct
  REGION 1..12: note = linker
RADAAAAGGPGS

SEQ ID NO: 340 - protein, 27 aa, synthetic construct
  REGION 1..27: note = linker
RADAAAAGGGGSGGGGSGGGGSGGGGS

SEQ ID NO: 341 - protein, 6 aa, synthetic construct
  REGION 1..6: note = linker
SAKTTP

SEQ ID NO: 342 - protein, 10 aa, synthetic construct
  REGION 1..10: note = linker
SAKTTPKLGG

SEQ ID NO: 343 - protein, 18 aa, synthetic construct
  REGION 1..18: note = linker
SAKTTPKLEEGEFSEARV

SEQ ID NO: 344 - protein, 5 aa, synthetic construct
  REGION 1..5: note = linker
ADAAP

SEQ ID NO: 345 - protein, 12 aa, synthetic construct
  REGION 1..12: note = linker
ADAAPTVSIFPP

SEQ ID NO: 346 - protein, 5 aa, synthetic construct
  REGION 1..5: note = linker
TVAAP

SEQ ID NO: 347 - protein, 12 aa, synthetic construct
  REGION 1..12: note = linker
TVAAPSVFIFPP

SEQ ID NO: 348 - protein, 6 aa, synthetic construct
  REGION 1..6: note = linker
QPKAAP

SEQ ID NO: 349 - protein, 13 aa, synthetic construct
  REGION 1..13: note = linker
QPKAAPSVTLFPP

SEQ ID NO: 350 - protein, 6 aa, synthetic construct
  REGION 1..6: note = linker
AKTTPP

SEQ ID NO: 351 - protein, 13 aa, synthetic construct
  REGION 1..13: note = linker
AKTTPPSVTPLAP

SEQ ID NO: 352 - protein, 6 aa, synthetic construct
  REGION 1..6: note = linker
AKTTAP

SEQ ID NO: 353 - protein, 13 aa, synthetic construct
  REGION 1..13: note = linker
AKTTAPSVYPLAP

SEQ ID NO: 354 - protein, 6 aa, synthetic construct
  REGION 1..6: note = linker
ASTKGP

SEQ ID NO: 355 - protein, 13 aa, synthetic construct
  REGION 1..13: note = linker
ASTKGPSVFPLAP

SEQ ID NO: 356 - protein, 15 aa, synthetic construct
  REGION 1..15: note = linker
GENKVEYAPALMALS

SEQ ID NO: 357 - protein, 15 aa, synthetic construct
  REGION 1..15: note = linker
GPAKELTPLKEAKVS

SEQ ID NO: 358 - protein, 15 aa, synthetic construct
  REGION 1..15: note = linker
GHEAAAVMQVQYPAS

(19) United States
(12) Patent Application Publication
(10) Pub. No.: US 2024/0190989 A1
(43) Pub. Date: Jun. 13, 2024

(54) ANTI-CEA AND ANTI-CD137 MULTISPECIFIC ANTIBODIES AND METHODS OF USE

(71) Applicant: BeiGene Switzerland GmbH, Basel (CH)

(72) Inventors: Liang QU, Beijing (CN); Tong ZHANG, Beijing (CN); Zhuo LI, Shanghai (CN); Xin CHEN, Beijing (CN); Lin ZHU, Beijing (CN); Penghao WANG, Beijing (CN); Xiaosui Zhou, Beijing (CN); Yuanyuan XIE, Beijing (CN); Jie LI, Shanghai (CN); Jian SUN, Beijing (CN); Jing SONG, Beijing (CN); Xuehui LI, Beijing (CN)

(73) Assignee: BeiGene Switzerland GmbH, Basel (CH)

(21) Appl. No.: 18/513,223

(22) Filed: Nov. 17, 2023

Related U.S. Application Data
(63) Continuation of application No. PCT/CN2022/093565, filed on May 18, 2022.

(30) Foreign Application Priority Data
May 21, 2021 (WO) PCT/CN2021/095113
Apr. 7, 2022 (WO) PCT/CN2022/085625
Apr. 21, 2022 (WO) PCT/CN2022/088172

(51) Int. Cl. C07K 16/30; A61K 39/00; A61P 35/00; C07K 16/28

(52) U.S. Cl. CPC C07K 16/3007; A61P 35/00; C07K 16/2878; A61K 2039/505; C07K 2317/31; C07K 2317/52; C07K 2317/565; C07K 2317/732; C07K 2317/734

(57) ABSTRACT

Multispecific antibodies and antigen-binding fragments thereof that bind to human CEA and CD137, a pharmaceutical composition comprising said antibody, and use of the multispecific antibody or the composition for treating.

### CROSS-REFERENCE TO RELATED APPLICATIONS

This application is a continuation of International Patent Application No. PCT/CN2022/093565, filed May 18, 2022, which claims priority from International Patent Application No. PCT/CN2021/095113, fled May 21, 2021, International Patent Application No. PCT/CN2022/085625, filed Apr. 7, 2022, and International Patent Application No. PCT/CN2022/088172, filed Apr. 21, 2022. The contents of these applications are incorporated herein by reference in their entirety.

### SEQUENCE LISTING

The instant application contains a Sequence Listing which has been submitted electronically in XML format, and is hereby incorporated by reference in its entirety. Said XML file was created on Jan. 3, 2024, is named 138881_0271_Sequence_Listing.xml, and is 582 bytes in size.

### FIELD OF THE DISCLOSURE

Disclosed herein are multispecific antibodies or antigen-binding fragments thereof that bind to human CEA and human CD137, a composition comprising said antibody, as well as methods of use for the treatment of cancer.

### BACKGROUND

Carcinoembryonic antigen (CEA, also known as CEACAM5 or CD66e) is a glycoprotein with a molecular weight of about 70-100 kDa depending on the amount of glycosylation present. The presence of CEA associated as cancer-specific antigen in human adenocarcinoma was first reported by Gold et al., J. Exp. Med., 121, 439 (1965). CEA is normally expressed in a variety of glandular epithelial tissues (such as the gastrointestinal, respiratory, and urogenital tracts) where it appears to be localized to the apical surface of the cells (Hammarstrom, S. Semin. Cancer Biol. 9, 67-81 (1999)). For example, it is found in the columnar epithelial and goblet cells of the colon (Fraengsmyr et al., Tumor Biol. 20:277-292(1999)). In tumors generated from these tissue types, CEA expression increases from the apical membrane to the cell surface and once removed from the cell surface, enters into the bloodstream (Hammarstrom, S. Semin. Cancer Biol. 9, 67-81; (1999) see also Fraengsmyr et al., Tumor Biol. 20:277-292(1999)). CEA overexpression was observed in many types of cancers, including colorectal cancer, pancreatic cancer, lung cancer, gastric cancer, hepatocellular carcinoma, breast cancer, and thyroid cancer. Therefore, CEA has been useful as a diagnostic tumor marker to determine the elevated levels of CEA in the blood of cancer patients in the prognosis and management of cancer (Chevinsky, A. H. (1991) Semin. Surg. Oncol. 7, 162-166; Shively, J. E. et al., (1985) Crit. Rev. Oncol. Hematol. 2, 355-399).

CEA has been considered as a useful tumor-associated antigen for targeted therapy (Kuroki M, et al., (2002) Anticancer Res 22:4255-64). One approach was the generation of retrovirus constructs that displayed an anti-CEA scFv, and would deliver a nitric oxide synthase (iNOS) gene to CEA expressing cancer cells. (Kuroki M. et al., (2000) Anticancer Res. 20(6A):4067-71). Another approach was to attach radioisotopes to anti-CEA antibodies and demonstrate that radiation was directed specifically at the CEA expressing tumor (Wilkinson et al., PNAS USA 98, 10256-60 (2001), Goldenberg et al., Am. J. Gastroenterol., 86: 1392-1403 (1991), Olafsen T. et al., Protein Engineering, Design & Selection, 17, 21-27, (2004), Meyer et al., Clin. Cancer Res. 15:4484-4492 (2009), Sharkey et al., J. Nucl. Med. 46:620-633 (2005)). The radioisotope approach has been extended to anti-CEA antibody drug conjugates (ADC). For example, Shinmi et al., reported on an anti-CEA antibody conjugated to monomethyl auristatin E (MMAE) (Shinmi et al., Cancer Med. 6(4): 798-808 (2017)).

However, one of the issues for anti-CEA antibodies is that of cross-reactivity. CEA is highly homologous to other CEACAM family members, for example, human CEA shows 84% homology with CEACAM6, 77% homology with CEACAM8 and 73% identity with CEACAM1. The current disclosure provides for anti-CEA antibodies that are specific for CEA.

CD137 (also known as TNFRSF9/41BB) is a co-stimulatory molecule belonging to the TNFRSF family. It was discovered by T-cell-factor-screening on mouse helper and cytotoxic cells stimulated by concanavalin A and was identified in 1989 as an inducible gene that was expressed on antigen-primed T cells but not on resting ones (Kwon et al., Proc. Natl. Acad. Sci. USA. 1989; 86:1963-1967). CD137 is a co-stimulatory molecule belonging to the TNFRSF. It was discovered in the late 80s during T-cell-factor-screening on mouse helper and cytotoxic cells stimulated by concanavalin A. In addition, it is known to be expressed in dendritic (DCs), natural killer cells (NKs) (Vinay et al., Mol. Cancer Ther. 2012; 11:1062-1070), activated CD4+ and CD8+T lymphocytes, eosinophils, natural killer T cells (NKTs), and mast cells (Kwon et al., 1989 supra; Vinay D., Int. J. Hematol. 2006; 83:23-28).

The anti-CD137 antibodies Urelumab (BMS-663513) which binds to CRD I of CD137 and Utomilumab (PF-05082566) which binds to CRDs III and IV of CD137 show potential as cancer therapeutics for their ability to activate cytotoxic T cells and to increase the production of interferon gamma (IFN-7). The mechanisms underlying tumor regression by these antibodies are the effects on immune cells responses to cancer cells. Anti-CD137 antibody stimulates and activates effector T lymphocytes (e.g., stimulating CD8 T lymphocytes to produce INFT), NKTs, and APCs (e.g., macrophages).

Urelumab demonstrated promising results in preclinical experiments and early clinical studies (Sznol et al., Clin. Oncol. 2008; 26(Suppl. 15)). However, in later studies Urelumab demonstrated liver toxicity resulting the pausing development of the antibody until February 2012 (Segal et al., Clin. Cancer Res. 2017; 23:1929-1936). The liver toxicity was mostly due to S100A4 protein secreted by tumor and stromal cells, and studies that dose limited Urelumab to 8 mg or 0.1 mg/kg per patient for every 3 weeks has restored interest in this antibody (Segal et al., Clin. Cancer Res. 2017; 23:1929-1936).

In contrast with Urelumab, Utomilumab showed a better safety profile and initial studies show no liver toxicity or other dose limiting factors (Segal et al., J. Clin. Oncol. 2014; 32(Suppl. 15)). The reported outcomes from a phase I trial of Utomulumab as monotherapy indicated a good safety profile (Segal et al., Clin. Cancer Res. 2018; 24:1816-1823). The difference between the two antibodies has been speculated to be due to their different binding sites on the CD137 receptor.

Given the unique biology of both of these targets, anti-CEA×CD137 multispecific antibodies that recruit immune cells to CEA expressing cancers would be useful in the treatment of cancer.

### SUMMARY OF THE DISCLOSURE

The present disclosure is directed to multispecific anti-CEA×CD137 antibodies and antigen-binding fragments thereof. The present disclosure encompasses the following embodiments.

A multispecific antibody or antigen-binding fragment thereof, comprising a first antigen binding domain that specifically binds to human CEA at amino acids 596 to 674 of SEQ ID NO:88 and a second antigen binding domain that specifically binds to human CD137.

The multispecific antibody or antigen-binding fragment, wherein the first antigen binding domain does not bind to other CEACAM family members.

The multispecific antibody or antigen-binding fragment, wherein the first antigen binding domain that specifically binds to human CEA comprises:
(i) a heavy chain variable region that comprises (a) a HCDR1 (Heavy Chain Complementarity Determining Region 1) of SEQ ID NO: 7, (b) a HCDR2 of SEQ ID NO:8, (c) a HCDR3 of SEQ ID NO:9 and a light chain variable region that comprises: (d) a LCDR1 (Light Chain Complementarity Determining Region 1) of SEQ ID NO:10, (e) a LCDR2 of SEQ ID NO:11, and (f) a LCDR3 of SEQ ID NO:6;
(ii) a heavy chain variable region that comprises (a) a HCDR1 of SEQ ID NO:24, (b) a HCDR2 of SEQ ID NO:25, (c) a HCDR3 of SEQ ID NO:26; and a light chain variable region that comprises: (d) a LCDR1 of SEQ ID NO:27, (e) a LCDR2 of SEQ ID NO:28, and (f) a LCDR3 of SEQ ID NO: 23; or
(iii) a heavy chain variable region that comprises (a) a HCDR1 of SEQ ID NO:41, (b) a HCDR2 of SEQ ID NO:42, (c) a HCDR3 of SEQ ID NO:43; and a light chain variable region that comprises: (d) a LCDR1 of SEQ ID NO:44, (e) a LCDR2 of SEQ ID NO:45, and (f) a LCDR3 of SEQ ID NO:40.

The multispecific antibody or antigen-binding fragment, comprising:
(i) a heavy chain variable region (VH) comprising an amino acid sequence at least 90, 91, 92, 93, 94, 95, 96, 97, 98 or 99% identical to SEQ ID NO:14, and a light chain variable region (VL) comprising an amino acid sequence at least 90, 91, 92, 93, 94, 95, 96, 97, 98 or 99% identical to SEQ ID NO: 15;
(ii) a heavy chain variable region (VH) comprising an amino acid sequence at least 90, 91, 92, 93, 94, 95, 96, 97, 98 or 99% identical to SEQ ID NO: 31, and a light chain variable region (VL) comprising an amino acid sequence at least 90, 91, 92, 93, 94, 95, 96, 97, 98 or 99% identical to SEQ ID NO: 32; or
(iii) a heavy chain variable region (VH) comprising an amino acid sequence at least 90, 91, 92, 93, 94, 95, 96, 97, 98 or 99% identical to SEQ ID NO: 48, and a light chain variable region (VL) comprising an amino acid sequence at least 90, 91, 92, 93, 94, 95, 96, 97, 98 or 99% identical to SEQ ID NO: 49.

The multispecific antibody or antigen-binding fragment, wherein one, two, three, four, five, six, seven, eight, nine, or ten amino acids within SEQ ID NO:14, 15, 31, 32, 48, or 49 have been inserted, deleted or substituted.

The multispecific antibody or antigen-binding fragment, wherein the first antigen binding domain comprises:
(i) a heavy chain variable region (VH) that comprises SEQ ID NO:14, and a light chain variable region (VL) that comprises SEQ ID NO: 15;
(ii) a heavy chain variable region (VH) that comprises SEQ ID NO: 31, and a light chain variable region (VL) that comprises SEQ ID NO: 32; or
(iii) a heavy chain variable region (VH) that comprises SEQ ID NO: 48, and a light chain variable region (VL) that comprises SEQ ID NO: 49.

The multispecific antibody or antigen-binding fragment, wherein the second antigen binding domain that specifically binds to human CD137 comprises:
(i) a heavy chain variable region that comprises (a) a HCDR1 (Heavy Chain Complementarity Determining Region 1) of SEQ ID NO: 65, (b) a HCDR2 of SEQ ID NO:80, (c) a HCDR3 of SEQ ID NO:81;
(ii) a heavy chain variable region that comprises (a) a HCDR1 of SEQ ID NO:65, (b) a HCDR2 of SEQ ID NO:73, (c) a HCDR3 of SEQ ID NO:67;
(iii) a heavy chain variable region that comprises (a) a HCDR1 of SEQ ID NO:65, (b) a HCDR2 of SEQ ID NO:66, (c) a HCDR3 of SEQ ID NO:67; or
(iv) a heavy chain variable region that comprises (a) a HCDR1 of SEQ ID NO:55, (b) a HCDR2 of SEQ ID NO:56, (c) a HCDR3 of SEQ ID NO:57.

The multispecific antibody or antigen-binding fragment, comprising:
(i) a heavy chain variable region (VH) comprising an amino acid sequence at least 90, 91, 92, 93, 94, 95, 96, 97, 98 or 99% identical to SEQ ID NO:84;
(ii) a heavy chain variable region (VH) comprising an amino acid sequence at least 90, 91, 92, 93, 94, 95, 96, 97, 98 or 99% identical to SEQ ID NO: 86;
(iii) a heavy chain variable region (VH) comprising an amino acid sequence at least 90, 91, 92, 93, 94, 95, 96, 97, 98 or 99% identical to SEQ ID NO:75;
(iv) a heavy chain variable region (VH) comprising an amino acid sequence at least 90, 91, 92, 93, 94, 95, 96, 97, 98 or 99% identical to SEQ ID NO:70; or
(v) a heavy chain variable region (VH) comprising an amino acid sequence at least 90, 91, 92, 93, 94, 95, 96, 97, 98 or 99% identical to SEQ ID NO:60.

The multispecific antibody or antigen-binding fragment, wherein one, two, three, four, five, six, seven, eight, nine, or ten amino acids within SEQ ID NO:84, 86, 75, 70, or 60 have been inserted, deleted or substituted.

The multispecific antibody or antigen-binding fragment, wherein the second antigen binding domain comprises:
(i) a heavy chain variable region (VH) that comprises SEQ ID NO: 84;
(ii) a heavy chain variable region (VH) that comprises SEQ ID NO: 86;
(iii) a heavy chain variable region (VH) that comprises SEQ ID NO: 75;
(iv) a heavy chain variable region (VH) that comprises SEQ ID NO: 70; or
(v) a heavy chain variable region (VH) that comprises SEQ ID NO: 60.

The multispecific antibody or antigen-binding fragment, wherein:
(i) the first antigen binding domain that specifically binds to human CEA comprises: a heavy chain variable region that comprises (a) a HCDR1 of SEQ ID NO: 7, (b) a HCDR2 of SEQ ID NO:8, (c) a HCDR3 of SEQ ID NO:9 and a light chain variable region that comprises: (d) a LCDR1 of SEQ ID NO:10, (e) a LCDR2 of SEQ ID NO:11, and (f) a LCDR3 of SEQ ID NO:6; and the second antigen binding domain that specifically binds to human CD137 comprises: a heavy chain variable region that comprises (a) a HCDR1 of SEQ ID NO: 65, (b) a HCDR2 of SEQ ID NO:80, (c) a HCDR3 of SEQ ID NO:81 and;
(ii) the first antigen binding domain that specifically binds to human CEA comprises: a heavy chain variable region that comprises (a) a HCDR1 of SEQ ID NO:7, (b) a HCDR2 of SEQ ID NO:8, (c) a HCDR3 of SEQ ID NO:9; and a light chain variable region that comprises: (d) a LCDR1 of SEQ ID NO:10, (e) a LCDR2 of SEQ ID NO:11, and (f) a LCDR3 of SEQ ID NO: 6; and the second antigen binding domain that specifically binds to human CD137 comprises: a heavy chain variable region that comprises (a) a HCDR1 of SEQ ID NO: 65, (b) a HCDR2 of SEQ ID NO:73, (c) a HCDR3 of SEQ ID NO:67;
(iii) the first antigen binding domain that specifically binds to human CEA comprises: a heavy chain variable region that comprises (a) a HCDR1 of SEQ ID NO:7, (b) a HCDR2 of SEQ ID NO:8, (c) a HCDR3 of SEQ ID NO:9; and a light chain variable region that comprises: (d) a LCDR1 of SEQ ID NO:10, (e) a LCDR2 of SEQ ID NO:11, and (f) a LCDR3 of SEQ ID NO:6; and the second antigen binding domain that specifically binds to human CD137 comprises: a heavy chain variable region that comprises (a) a HCDR1 of SEQ ID NO: 65, (b) a HCDR2 of SEQ ID NO:66, (c) a HCDR3 of SEQ ID NO:67; or
(iv) the first antigen binding domain that specifically binds to human CEA comprises: a heavy chain variable region that comprises (a) a HCDR1 of SEQ ID NO: 7, (b) a HCDR2 of SEQ ID NO:8, (c) a HCDR3 of SEQ ID NO:9 and a light chain variable region that comprises: (d) a LCDR1 of SEQ ID NO:10, (e) a LCDR2 of SEQ ID NO:11, and (f) a LCDR3 of SEQ ID NO:6; and the second antigen binding domain that specifically binds to human CD137 comprises: a heavy chain variable region that comprises (a) a HCDR1 of SEQ ID NO:55, (b) a HCDR2 of SEQ ID NO:56, (c) a HCDR3 of SEQ ID NO:57.

The multispecific antibody or antigen-binding fragment, wherein:
(i) the first antigen binding domain that specifically binds to human CEA comprises: a heavy chain variable region (VH) that comprises SEQ ID NO:14, and a light chain variable region (VL) that comprises SEQ ID NO: 15; and the second antigen binding domain that specifically binds to human CD137 comprises: a heavy chain variable region (VH) that comprises SEQ ID NO:84;
(ii) the first antigen binding domain that specifically binds to human CEA comprises: a heavy chain variable region (VH) that comprises SEQ ID NO:14, and a light chain variable region (VL) that comprises SEQ ID NO: 15; and the second antigen binding domain that specifically binds to human CD137 comprises: a heavy chain variable region (VH) that comprises SEQ ID NO:86;
(iii) the first antigen binding domain that specifically binds to human CEA comprises: a heavy chain variable region (VH) that comprises SEQ ID NO:14, and a light chain variable region (VL) that comprises SEQ ID NO: 15; and the second antigen binding domain that specifically binds to human CD137 comprises: a heavy chain variable region (VH) that comprises SEQ ID NO:75;
(iv) the first antigen binding domain that specifically binds to human CEA comprises: a heavy chain variable region (VH) that comprises SEQ ID NO:14, and a light chain variable region (VL) that comprises SEQ ID NO: 15; and the second antigen binding domain that specifically binds to human CD137 comprises: a heavy chain variable region (VH) that comprises SEQ ID NO:70; or
(v) the first antigen binding domain that specifically binds to human CEA comprises: a heavy chain variable region (VH) that comprises SEQ ID NO:14, and a light chain variable region (VL) that comprises SEQ ID NO: 15; and the second antigen binding domain that specifically binds to human CD137 comprises: a heavy chain variable region (VH) that comprises SEQ ID NO:60.

The multispecific antibody or antigen-binding fragment, which is a monoclonal antibody, a chimeric antibody, a humanized antibody, a human engineered antibody, a single chain antibody (scFv), a Fab fragment, a Fab′ fragment, or a F(ab′)2 fragment.

The multispecific antibody, wherein the multispecific antibody is a bispecific antibody.

The bispecific antibody, wherein the bispecific antibody contains a linker from SEQ ID NO:317 to SEQ ID NO 358.

The bispecific antibody, wherein the linker is SEQ ID NO: 324.

The bispecific antibody, wherein the linker is SEQ ID NO: 329.

The bispecific antibody, wherein the multispecific antibody is BE-146 (SEQ ID NO: 313 and SEQ ID NO: 179).

The bispecific antibody, wherein the multispecific antibody is BE-189 (SEQ ID NO: 255 and SEQ ID NO: 179).

The bispecific antibody, wherein the multispecific antibody is BE-718 (SEQ ID NO: 295 and SEQ ID NO: 179).

The bispecific antibody, wherein the multispecific antibody is BE-740 (SEQ ID NO: 297 and SEQ ID NO: 179).

The bispecific antibody, wherein the multispecific antibody is BE-942 (SEQ ID NO: 299, SEQ ID NO: 301 and SEQ ID NO:303).

The bispecific antibody, wherein the multispecific antibody is BE-755 (SEQ ID NO: 299, SEQ ID NO: 301 and SEQ ID NO: 305).

The bispecific antibody, wherein the multispecific antibody is BE-562 (SEQ ID NO: 307 and SEQ ID NO: 179).

The bispecific antibody, wherein the multispecific antibody is BE-375 (SEQ ID NO: 309 and SEQ ID NO: 179).

The bispecific antibody, wherein the multispecific antibody is BE-244 (SEQ ID NO: 311 and SEQ ID NO: 179).

The multispecific antibody or antigen-binding fragment, wherein the antibody or antigen-binding fragment thereof has antibody dependent cellular cytotoxicity (ADCC) or complement dependent cytotoxicity (CDC).

The multispecific antibody or antigen-binding fragment, wherein the antibody or antigen-binding fragment thereof has reduced glycosylation or no glycosylation or is hypofucosylated.

The multispecific antibody or antigen-binding fragment, wherein the antibody or antigen-binding fragment thereof comprises increased bisecting GlcNac structures.

The multispecific antibody or antigen-binding fragment, wherein the Fc domain is an IgG1 with reduced effector function.

The multispecific antibody or antigen-binding fragment, wherein the Fc domain is an IgG4.

The multispecific antibody or antigen-binding fragment, wherein the IgG4 has an S228P substitution (according to EU numbering system).

A pharmaceutical composition comprising the multispecific antibody or antigen-binding fragment thereof, further comprising a pharmaceutically acceptable carrier.

The pharmaceutical composition, further comprising histidine/histidine HCl, trehalose dihydrate, and polysorbate 20.

A method of treating cancer comprising administering to a patient in need an effective amount of the multispecific antibody or antigen-binding fragment.

The method, wherein the cancer is gastric cancer, colon cancer, pancreatic cancer, breast cancer, head and neck cancer, kidney cancer, liver cancer, lung cancer, small cell lung cancer, non-small cell lung cancer, ovarian cancer, skin cancer, mesothelioma, lymphoma, leukemia, myeloma and sarcoma.

The method, wherein the colon cancer is colorectal cancer.

The method wherein the gastric cancer is associated with high CEA levels in the serum.

The method, wherein the lung cancer is associated with high CEA levels in the serum.

The method, wherein the non-small cell lung cancer is associated with high CEA levels in the serum.

The method of treatment, wherein the multispecific antibody is administered at a range of 5 mg-1200 mg.

The method, wherein the multispecific antibody is administered at a range of 5 mg-1200 mg, once per week.

The method, wherein the multispecific antibody or antigen-binding fragment is administered in combination with another therapeutic agent.

The method, wherein the therapeutic agent is paclitaxel or a paclitaxel agent, docetaxel, carboplatin, topotecan, cisplatin, irinotecan, doxorubicin, lenalidomide or 5-azacytidine.

The method, wherein the therapeutic agent is a paclitaxel agent, lenalidomide or 5-azacytidine.

The method, wherein the therapeutic agent an anti-PD1 or anti-PDL1 antibody.

The method, wherein the anti-PD1 antibody is Tislelizumab.

An isolated nucleic acid that encodes the multispecific antibody or antigen-binding fragment.

A vector comprising the nucleic acid.

A host cell comprising the nucleic acid or the vector.

A process for producing a multispecific antibody or antigen-binding fragment thereof comprising cultivating the host cell and recovering the antibody or antigen-binding fragment from the culture.

In one embodiment, the multispecific antibody or an antigen-binding fragment thereof comprises one or more complementarity determining regions (CDRs) comprising an amino acid sequence selected from a group consisting of SEQ ID NO:7, SEQ ID NO:8, SEQ ID NO:9, SEQ ID NO:10, SEQ ID NO:11, SEQ ID NO:6, SEQ ID NO:24, SEQ ID NO:25, SEQ ID NO:26, SEQ ID NO:27, SEQ ID NO:28, SEQ ID NO:23, SEQ ID NO:41, SEQ ID NO:42, SEQ ID NO:43, SEQ ID NO:44, SEQ ID NO:45, SEQ ID NO:40, SEQ ID NO:55, SEQ ID NO:56, SEQ ID NO: 57, SEQ ID NO:66, SEQ ID NO:66, SEQ ID NO:67, SEQ ID NO:73, SEQ ID NO:67, SEQ ID NO: 74, SEQ ID NO:65, SEQ ID NO:80, or SEQ ID NO:81.

In another embodiment, the multispecific antibody or an antigen-binding fragment thereof comprises: (a) a heavy chain variable region comprising one or more complementarity determining regions (HCDRs) comprising an amino acid sequence selected from the group consisting of SEQ ID NO: 7, SEQ ID NO: 8, SEQ ID NO: 9, SEQ ID NO: 24, SEQ ID NO: 25, SEQ ID NO: 26, SEQ ID NO: 41, SEQ ID NO: 42, SEQ ID NO: 43; SEQ ID NO: 55, SEQ ID NO: 56, SEQ ID NO: 57, SEQ ID NO:65, SEQ ID NO:66, SEQ ID NO:67, SEQ ID NO: 65, SEQ ID NO: 73, SEQ ID NO: 67, SEQ ID NO:65, SEQ ID NO:80 and SEQ ID NO:81 and/or (b) a light chain variable region comprising one or more complementarity determining regions (LCDRs) having an amino acid sequence selected from the group consisting of SEQ ID NO: 10, SEQ ID NO: 11, SEQ ID NO: 6, SEQ ID NO: 27, SEQ ID NO: 28, SEQ ID NO: 23, SEQ ID NO: 44 SEQ ID NO: 45 and SEQ ID NO: 40.

In another embodiment, the multispecific antibody or an antigen-binding fragment thereof comprises: (a) a heavy chain variable region comprising three complementarity determining regions (HCDRs) which are HCDR1 comprising an amino acid sequence of SEQ ID NO: 7; SEQ ID NO: 24, SEQ ID NO:41, SEQ ID NO: 55, SEQ ID NO:65, SEQ ID NO: 68, SEQ ID NO:72 or SEQ ID NO:77, HCDR2 comprising an amino acid sequence of SEQ ID NO: 8, SEQ ID NO: 25, SEQ ID NO: 42, SEQ ID NO: 56, SEQ ID NO:66, SEQ ID NO: 73, or SEQ ID NO:80, and HCDR3 comprising an amino acid sequence of SEQ ID NO: 9, SEQ ID NO: 26, SEQ ID NO:43, SEQ ID NO: 57, SEQ ID NO: 67, or SEQ ID NO: 81, and/or (b) a light chain variable region comprising three complementarity determining regions (LCDRs) which are LCDR1 comprising an amino acid sequence of SEQ ID NO: 10, SEQ ID NO: 27, or SEQ ID NO: 44, LCDR2 comprising an amino acid sequence of SEQ ID NO: 11, SEQ ID NO: 28, or SEQ ID NO:45; and LCDR3 comprising an amino acid sequence of SEQ ID NO: 6, SEQ ID NO: 23, SEQ ID NO:40.

In another embodiment, the multispecific antibody or an antigen-binding fragment thereof comprises: (a) a heavy chain variable region comprising three complementarity determining regions (HCDRs) which are HCDR1 comprising an amino acid sequence of SEQ ID NO: 7, HCDR2 comprising an amino acid sequence of SEQ ID NO: 8, and HCDR3 comprising an amino acid sequence of SEQ ID NO: 9; or HCDR1 comprising an amino acid sequence of SEQ ID NO: 24, HCDR2 comprising an amino acid sequence of SEQ ID NO: 25, and HCDR3 comprising an amino acid sequence of SEQ ID NO: 26; or HCDR1 comprising an amino acid sequence of SEQ ID NO: 41, HCDR2 comprising an amino acid sequence of SEQ ID NO: 42, and HCDR3 comprising an amino acid sequence of SEQ ID NO: 43; HCDR1 comprising an amino acid sequence of SEQ ID NO: 55, HCDR2 comprising an amino acid sequence of SEQ ID NO: 56, and HCDR3 comprising an amino acid sequence of SEQ ID NO: 57; HCDR1 comprising an amino acid sequence of SEQ ID NO:65, HCDR2 comprising an amino acid sequence of SEQ ID NO: 66, and HCDR3 comprising an amino acid sequence of SEQ ID NO:67; or HCDR1 comprising an amino acid sequence of SEQ ID NO: 65, HCDR2 comprising an amino acid sequence of SEQ ID NO: 73, and HCDR3 comprising an amino acid sequence of SEQ ID NO:67, HCDR1 comprising an amino acid sequence of SEQ ID NO: 65, HCDR2 comprising an amino acid sequence of SEQ ID NO: 80, and HCDR3 comprising an amino acid sequence of SEQ ID NO:81 and/or (b) a light chain variable region comprising three complementarity determining regions (LCDRs) which are LCDR1 comprising an amino acid sequence of SEQ ID NO: 10, LCDR2 comprising an amino acid sequence of SEQ ID NO: 11, and LCDR3 comprising an amino acid sequence of SEQ ID NO: 6; or LCDR1 comprising an amino acid sequence of SEQ ID NO: 27, LCDR2 comprising an amino acid sequence of SEQ ID NO: 28, and LCDR3 comprising an amino acid sequence of SEQ ID NO: 23; or LCDR1 comprising an amino acid sequence of SEQ ID NO: 44, LCDR2 comprising an amino acid sequence of SEQ ID NO: 45, and LCDR3 comprising an amino acid sequence of SEQ ID NO: 40.

In another embodiment, the multispecific antibody or the antigen-binding fragment comprises: a first antigen binding domain comprising: a heavy chain variable region that comprises (a) a HCDR1 of SEQ ID NO: 7, (b) a HCDR2 of SEQ ID NO:8, (c) a HCDR3 of SEQ ID NO:9 and a light chain variable region that comprises: (d) a LCDR1 of SEQ ID NO:10, (e) a LCDR2 of SEQ ID NO:11, and (f) a LCDR3 of SEQ ID NO:6; and a second antigen binding domain comprising: a heavy chain variable region that comprises (a) a HCDR1 of SEQ ID NO: 65, (b) a HCDR2 of SEQ ID NO:80, (c) a HCDR3 of SEQ ID NO:81.

In another embodiment, the multispecific antibody or the antigen-binding fragment comprises: a first antigen binding domain comprises: a heavy chain variable region that comprises (a) a HCDR1 of SEQ ID NO:24, (b) a HCDR2 of SEQ ID NO:25, (c) a HCDR3 of SEQ ID NO:26; and a light chain variable region that comprises: (d) a LCDR1 of SEQ ID NO:27, (e) a LCDR2 of SEQ ID NO:28, and (f) a LCDR3 of SEQ ID NO: 23; and a second antigen binding domain comprises: a heavy chain variable region that comprises (a) a HCDR1 of SEQ ID NO: 65, (b) a HCDR2 of SEQ ID NO:80, (c) a HCDR3 of SEQ ID NO:81.

In yet another embodiment, the multispecific antibody or the antigen-binding fragment comprises: a first antigen binding domain comprises: a heavy chain variable region that comprises (a) a HCDR1 of SEQ ID NO:41, (b) a HCDR2 of SEQ ID NO:42, (c) a HCDR3 of SEQ ID NO:43; and a light chain variable region that comprises: (d) a LCDR1 of SEQ ID NO:44, (e) a LCDR2 of SEQ ID NO:45, and (f) a LCDR3 of SEQ ID NO:40; and a second antigen binding domain comprises: a heavy chain variable region that comprises ((a) a HCDR1 of SEQ ID NO: 65, (b) a HCDR2 of SEQ ID NO:80, (c) a HCDR3 of SEQ ID NO:81.

In yet another embodiment, the multispecific antibody or the antigen-binding fragment comprises: a first antigen binding domain comprises: a heavy chain variable region that comprises (a) a HCDR1 of SEQ ID NO: 7, (b) a HCDR2 of SEQ ID NO:8, (c) a HCDR3 of SEQ ID NO:9 and a light chain variable region that comprises: (d) a LCDR1 of SEQ ID NO:10, (e) a LCDR2 of SEQ ID NO:11, and (f) a LCDR3 of SEQ ID NO:6; and a second antigen binding domain comprises: a heavy chain variable region that comprises (a) a HCDR1 of SEQ ID NO:65, (b) a HCDR2 of SEQ ID NO:73, and (c) a HCDR3 of SEQ ID NO:67.

In another embodiment, the multispecific antibody or the antigen-binding fragment comprises: a first antigen binding domain comprises: a heavy chain variable region that comprises (a) a HCDR1 of SEQ ID NO: 7, (b) a HCDR2 of SEQ ID NO:8, (c) a HCDR3 of SEQ ID NO:9 and a light chain variable region that comprises: (d) a LCDR1 of SEQ ID NO:10, (e) a LCDR2 of SEQ ID NO:11, and (f) a LCDR3 of SEQ ID NO:6; and a second antigen binding domain comprises: a heavy chain variable region that comprises (a) a HCDR1 of SEQ ID NO:65, (b) a HCDR2 of SEQ ID NO:66, and (c) a HCDR3 of SEQ ID NO:67.

In another embodiment, the multispecific antibody or the antigen-binding fragment comprises: a first antigen binding domain comprises: a heavy chain variable region that comprises (a) a HCDR1 of SEQ ID NO: 7, (b) a HCDR2 of SEQ ID NO:8, (c) a HCDR3 of SEQ ID NO:9 and a light chain variable region that comprises: (d) a LCDR1 of SEQ ID NO:10, (e) a LCDR2 of SEQ ID NO:11, and (f) a LCDR3 of SEQ ID NO:6; and a second antigen binding domain comprises: a heavy chain variable region that comprises (a) a HCDR1 of SEQ ID NO:55, (b) a HCDR2 of SEQ ID NO:56, and (c) a HCDR3 of SEQ ID NO:57.

In another embodiment, the multispecific antibody or the antigen-binding fragment comprises: a first antigen binding domain comprises: a heavy chain variable region that comprises (a) a HCDR1 of SEQ ID NO: 41, (b) a HCDR2 of SEQ ID NO:42, (c) a HCDR3 of SEQ ID NO:43 and a light chain variable region that comprises: (d) a LCDR1 of SEQ ID NO:44, (e) a LCDR2 of SEQ ID NO:45, and (f) a LCDR3 of SEQ ID NO:40; and a second antigen binding domain comprising:
(i) a heavy chain variable region that (a) a HCDR1 of SEQ ID NO:65, (b) a HCDR2 of SEQ ID NO:80, (c) a HCDR3 of SEQ ID NO:81;
(ii) a heavy chain variable region that (a) a HCDR1 of SEQ ID NO:65, (b) a HCDR2 of SEQ ID NO:73, (c) a HCDR3 of SEQ ID NO:67;
(iii) a heavy chain variable region that (a) a HCDR1 of SEQ ID NO:65, (b) a HCDR2 of SEQ ID NO:66, (c) a HCDR3 of SEQ ID NO:67; or
(iv) a heavy chain variable region that (a) a HCDR1 of SEQ ID NO:55, (b) a HCDR2 of SEQ ID NO:56, (c) a HCDR3 of SEQ ID NO:57.

In another embodiment, the multispecific antibody or the antigen-binding fragment comprises: a first antigen binding domain comprises: a heavy chain variable region that comprises (a) a HCDR1 of SEQ ID NO: 24, (b) a HCDR2 of SEQ ID NO:25, (c) a HCDR3 of SEQ ID NO:26 and a light chain variable region that comprises: (d) a LCDR1 of SEQ ID NO:27, (e) a LCDR2 of SEQ ID NO:28, and (f) a LCDR3 of SEQ ID NO:23; and a second antigen binding domain comprising:
(i) a heavy chain variable region that (a) a HCDR1 of SEQ ID NO:65, (b) a HCDR2 of SEQ ID NO:80, (c) a HCDR3 of SEQ ID NO:81;
(ii) a heavy chain variable region that (a) a HCDR1 of SEQ ID NO:65, (b) a HCDR2 of SEQ ID NO:73, (c) a HCDR3 of SEQ ID NO:67;
(iii) a heavy chain variable region that (a) a HCDR1 of SEQ ID NO:65, (b) a HCDR2 of SEQ ID NO:66, (c) a HCDR3 of SEQ ID NO:67; or
(iv) a heavy chain variable region that (a) a HCDR1 of SEQ ID NO:55, (b) a HCDR2 of SEQ ID NO:56, (c) a HCDR3 of SEQ ID NO:57.

In one embodiment, the antibody of the present disclosure or an antigen-binding fragment thereof comprises: (a) a heavy chain variable region having an amino acid sequence of SEQ ID NO: 14, SEQ ID NO: 31, SEQ ID NO: 48, SEQ ID NO:60, SEQ ID NO: 70, SEQ ID NO:84, or SEQ ID NO:86 or an amino acid sequence at least 95%, 96%, 97%, 98% or 99% identical to any one of SEQ ID NO: 14, SEQ ID NO: 31, SEQ ID NO: 48, SEQ ID NO:60, SEQ ID NO: 70, SEQ ID NO:84, or SEQ ID NO:86; and/or (b) a light chain variable region comprising an amino acid sequence of SEQ ID NO: 15, SEQ ID NO: 32, or SEQ ID NO: 49, or an amino acid sequence at least 95%, 96%, 97%, 98% or 99% identical to any one of SEQ ID NO: 15, SEQ ID NO: 32, or SEQ ID NO: 49.

In another embodiment, the multispecific antibody of the present disclosure or an antigen-binding fragment thereof comprises: (a) a heavy chain variable region comprising an amino acid sequence of SEQ ID NO: 14, SEQ ID NO: 31, SEQ ID NO: 48, SEQ ID NO:60, SEQ ID NO: 70, SEQ ID NO:84, or SEQ ID NO:86 or an amino acid sequence comprising one, two, or three amino acid substitutions in the amino acid sequence of SEQ ID NO: 14, SEQ ID NO: 31, SEQ ID NO: 48, SEQ ID NO:60, SEQ ID NO: 70, SEQ ID NO:84, or SEQ ID NO:86; and/or (b) a light chain variable region comprising an amino acid sequence of SEQ ID NO: 15, SEQ ID NO: 32, or SEQ ID NO: 49 or an amino acid sequence comprising one, two, three, four, or five amino acid substitutions in the amino acid of SEQ ID NO: 15, SEQ ID NO: 32, or SEQ ID NO: 49. In another embodiment, the amino acid substitutions are conservative amino acid substitutions.

In one embodiment, the multispecific antibody or antigen-binding fragment comprises: a first antigen binding domain comprises: a heavy chain variable region (VH) that comprises SEQ ID NO:14, and a light chain variable region (VL) that comprises SEQ ID NO: 15; and a second antigen binding domain comprising:
(i) a VH that comprises SEQ ID NO: 84;
(ii) a VH that comprises SEQ ID NO: 86;
(iii) a VH that comprises SEQ ID NO:75;
(iv) a VH that comprises SEQ ID NO:70; or
(v) a VH that comprises SEQ ID NO:60.

In another embodiment, the multispecific antibody or antigen-binding fragment comprises: a first antigen binding domain comprises: a VH that comprises SEQ ID NO: 31, and a VL that comprises SEQ ID NO: 32; and a second antigen binding domain comprising:
(i) a VH that comprises SEQ ID NO:84;
(ii) a VH that comprises SEQ ID NO: 86;
(iii) a VH that comprises SEQ ID NO:75;
(iv) a VH that comprises SEQ ID NO:70; or
(v) a VH that comprises SEQ ID NO:60.

In another embodiment, the multispecific antibody or antigen-binding fragment comprises: a VH that comprises SEQ ID NO: 48, and a VL that comprises SEQ ID NO: 49 and a second antigen binding domain comprising:
(i) a VH that comprises SEQ ID NO:84;
(ii) a VH that comprises SEQ ID NO: 86;
(iii) a VH that comprises SEQ ID NO:75;
(iv) a VH that comprises SEQ ID NO:70; or
(v) a VH that comprises SEQ ID NO:60.

In one embodiment, the multispecific antibody of the present disclosure is of IgG1, IgG2, IgG3, or IgG4 isotype. In a more specific embodiment, the antibody of the present disclosure comprises Fc domain of wild-type human IgG1 (also referred as human IgG1 wt or huIgG1) or IgG2. In another embodiment, the antibody of the present disclosure comprises Fc domain of human IgG4 with S228P and/or R409K substitutions (according to EU numbering system).

In one embodiment, the multispecific antibody of the present disclosure binds to CEA with a binding affinity (K_{D}) of from 1×10⁻⁶ M to 1×10⁻¹⁰ M. In another embodiment, the antibody of the present disclosure binds to CEA with a binding affinity (K_{D}) of about 1×10⁻⁶ M, about 1×10⁻⁷ M, about 1×10⁻⁸ M, about 1×10⁻⁹ M or about 1×10⁻¹⁰ M.

In another embodiment, the anti-human CEA multispecific antibody of the present disclosure shows a cross-species binding activity to cynomolgus CEA.

In one embodiment, antibodies of the present disclosure have strong Fc-mediated effector functions. The antibodies mediate antibody-dependent cellular cytotoxicity (ADCC) against CEA expressing target cells.

The present disclosure relates to isolated nucleic acids comprising nucleotide sequences encoding the amino acid sequence of the multispecific antibody or antigen-binding fragment. In one embodiment, the isolated nucleic acid comprises a VH nucleotide sequence of SEQ ID NO: 16, SEQ ID NO: 33, SEQ ID NO: 50, SEQ ID NO: 61, SEQ ID NO: 71, SEQ ID NO: 76, SEQ ID NO: 85 or SEQ ID NO: 87 or a nucleotide sequence comprising at least 95%, 96%, 97%, 98% or 99% identity to SEQ ID NO: 16, SEQ ID NO: 33, SEQ ID NO: 50, SEQ ID NO: 61, SEQ ID NO: 71, SEQ ID NO: 76, SEQ ID NO: 85 or SEQ ID NO: 87, and encodes the VH region of the antibody or an antigen-binding fragment of the present disclosure. Alternatively or additionally, the isolated nucleic acid comprises a VL nucleotide sequence of SEQ ID NO: 17, SEQ ID NO: 34, or SEQ ID NO: 51 or a nucleotide sequence comprising at least 95%, 96%, 97%, 98% or 99% identity to SEQ ID NO: 17, SEQ ID NO: 34, or SEQ ID NO: 51, and encodes the VL region the antibody or an antigen-binding fragment of the present disclosure.

In another aspect, the present disclosure relates to a pharmaceutical composition comprising the CEA×CD137 multispecific antibody or antigen-binding fragment thereof, and optionally a pharmaceutically acceptable excipient.

In yet another aspect, the present disclosure relates to a method of treating a disease in a subject, which comprises administering the CEA×CD137 multispecific antibody or antigen-binding fragment thereof, or a CEA×CD137 multispecific antibody pharmaceutical composition in a therapeutically effective amount to a subject in need thereof. In another embodiment the disease to be treated by the antibody or the antigen-binding fragment is cancer.

The current disclosure relates to use of the CEA×CD137 multispecific antibody or the antigen-binding fragment thereof, or a CEA×CD137 multispecific antibody pharmaceutical composition for treating a disease, such as cancer.

### BRIEF DESCRIPTION OF THE DRAWINGS

FIG. 1 shows schematic diagrams of shed CEA (sCEA), chimeric CEA (CHIM), CEACAM6 and CEA variants (CEA-v). In CEA, domains N, A1, B1, A2, B2, A3, B3 and GPI linker (GPI) are labeled; in CEACAM6, domains N′, A′ and B′ are labeled.

FIG. 2A-B depicts phylogenetic trees of anti-CEA domain B3 antibody VH (FIG. 2A) and VL (FIG. 2B) regions. The VH and VL sequences of candidate anti-CEA antibodies were aligned using DNASTAR's Megalign™ software. Sequence homology was displayed in phylogenetic trees.

FIG. 3A shows affinity determination of purified murine anti-CEA antibody BGA13 on a chimeric construct (CHIM) by surface plasmon resonance (SPR). FIG. 3B depicts binding profiles of BGA13 by antigen ELISA.

FIG. 4A-B shows effects of soluble CEA (sCEA) on CEA antibodies binding to MKN45 cells. FIG. 4A shows binding profiles of domain B3 antibodies in the presence or absence of soluble CEA (sCEA); FIG. 4B are the antibody binding profiles of FIG. 4A shown as a histogram.

FIG. 5A-B shows the randomization sites for generating an antibody library for affinity maturation of humanized BGA13 antibody light chain CDR (LCDR) regions (FIG. 5A) and heavy chain CDR (HCDR) regions (FIG. 5B).

FIG. 6 shows the amino acid changes of BGA13 light chain CDR regions after four rounds of selection.

FIG. 7 shows the binding to LOVO cells of affinity matured, humanized BGA13 variants by flow cytometry.

FIG. 8 is the binding to MKN45 cells of optimized, humanized BGA113 variants by flow cytometry.

FIGS. 9A and 9B demonstrate that there is no off-target binding of antibody BGA113K to various CEACAM family members by flow cytometry (FIG. 9A) and antigen ELISA (FIG. 9B).

FIG. 10 show the effects of soluble CEA on BGA113K binding to CEA expressing cells MKN45 cells in the presence of various concentrations of soluble CEA.

FIG. 11 demonstrates that antibody BGA113 kills cells by ADCC in vitro.

FIG. 12 depicts the reduction in tumor volume of a murine cancer model when treated with BGA113 antibody.

FIG. 13A is a summary of human anti-huCD137 VH domain antibodies identified from each sub-library. FIG. 13B is graphic phylogenetic trees of human anti-huCD137 VH domain antibodies from each sub-library. The VH sequences of candidate anti-huCD137 VH domain antibodies were aligned using DNASTAR's Megalign™ software. Sequence homology was displayed in phylogenetic trees.

FIG. 14A shows the schematic diagram of human Fc fusion VH antibody format (VH-Fc). VH domain antibodies were fused at the N terminal of an inert Fc (without FcγR-binding) with a GS4 linker in between. FIG. 14B shows a representative screening result using supernatants containing VH-Fc proteins, and FIG. 14C shows one of the clones, BGA-4712 has been demonstrated to be capable to stimulate IL-2 production in Hut78/huCD137 cells in a dose dependent manner.

FIG. 15A-15B is the binding profiles of a representative anti-huCD137 VH domain antibody BGA-4712. FIG. 15A depicts the determination of human anti-huCD137 VH domain antibody BGA-4712 binding by flow cytometry. FIG. 15B shows the blocking of human anti-huCD137 VH domain antibody BGA-4712 by huCD137 ligand (human CD137 ligand-ECD-mIgG2a fusion protein) interaction. The binding of purified human anti-huCD137 VH domain antibody BGA-4712 to CD137-expressing Hut78/huCD137 cells (Hut78/huCD137) was determined by flow cytometry.

FIG. 16A-D is a schematic diagram of CEA×CD137 multispecific antibody formats.

FIG. 17A-17B is a comparison on cell binding of CEA×CD137 multispecific antibodies by flow cytometry. FIG. 17A shows the binding to CEA-expressing cells CT26/CEA. FIG. 17B shows the binding to CD137-expressing cells Hut78/huCD137.

FIG. 18A-18B demonstrates that A-CD137/CEA stimulates PBMCs to produce IFN-γ in the presence of CEA⁺ tumor cells. FIG. 18A shows one of CEA×CD137 multispecific antibodies A-CEA/CD137 induces CD137 expressing cell line Hut78/huCD137 to produce Il-2. FIG. 18B shows one of CEA×CD137 multispecific antibodies A-CEA/CD137 induces human peripheral blood mononuclear cells (PBMCs) to produce IFN-γ in a dose dependent manner.

FIG. 19 shows the sequence of CDR regions of BGA-4712-M3 after four rounds of selections.

FIG. 20 is a binding assay of anti-huCD137 VH domain Ab BGA-5623 by flow cytometry, demonstrating that binding to CD137 is improved after affinity maturation.

FIG. 21 demonstrates no off-target binding of BGA-5623 on other TNF Receptor family members by ELISA.

FIG. 22A-22B shows the epitope mapping of human anti-huCD137 VH domain antibody BGA-5623. FIG. 22A is a representative screening result in a cell based binding assay. Expression of huCD137 mutants was monitored by Urelumab analog. FIG. 22B shows BGA-5623 binding of purified huCD137 mutants.

FIG. 23A demonstrates CD137 ligand competes with human anti-huCD137 VH domain antibody BGA-5623 via ELISA. FIG. 23B demonstrates an CD137×CEA multispecific antibody BGA-5623 could reduce CD137/CD137 ligand interaction in a cell-based ligand competition assay.

FIG. 24 shows partially competitive binding of VH (BGA-5623) against CD137L for CD137. The crystal structure of VH (BGA-5623)/CD137 was superposed with CD137L/CD137 complex (PDB: 6MGP) via CD137. The CD137, CD137L and VH are colored in black, white and grey, respectively.

FIG. 25 shows CDR3 of VH (BGA-5623) undergoes dramatically conformation change upon CD137 binding. The CD137 bound VH (BGA-5623) in black was superposed with apoVH (BGA-5623) in white.

FIG. 26 shows the atomic interactions on the binding surface of VH (BGA-5623)/CD137 complex. The binding interface between VH (BGA-5623) and CD137 identifies certain key residues of BGA-5623 (paratope residues) and CD137 (epitope residues). The CRD1 and 2 domains of CD137 are shown in grey cartoon covered with white transparent surface. The paratope residues is colored in black.

FIG. 27 is a schematic diagram of CEA×CD137 multispecific antibody formats for investigating other parameters, such as module ratio which might influence CD137 activation in vitro.

FIG. 28A-B demonstrates the bispecific antibody A-41A11-41A11 with a module ratio of 2:4 could activate CD137, no matter if CEA⁺ tumor cells are present (28A) or not (28B).

FIG. 29 is a schematic diagram of CEA×CD137 multispecific antibody formats for investigating other parameters, such as Fc functions and module orientation which might influence CD137 activation in vitro.

FIG. 30A demonstrates that studied CEA×CD137 multispecific antibodies only stimulate PBMCs to produce IFN-γ in the presence of CEA⁺ tumor cells. FIG. 30B shows no IFN-γ was induced by CEA×CD137 multispecific antibodies in the absence of CEA⁺ tumor cells.

FIG. 31 demonstrates that the linker length has minimal influence on CD137 activation in vitro in the presence of CEA⁺ tumor cells.

FIG. 32A-D shows Format A-BGA-5623(BE-189) (FIG. 32 B) induces significant inhibition of tumor growth in vivo, but not A-IgG1-BGA-5623 (BE-740) (FIG. 32 C) with Urelumab as a comparison (FIG. 32 D).

FIG. 33 is a schematic diagram of designed tumor-targeted CEA×CD137 multispecific antibody format.

FIG. 34A-34B shows antigen binding ELISA of BE-146 to huCEA (FIG. 34A) and huCD137-mIgG2a (FIG. 34B). Two batches of BE-146 were tested in this assay.

FIG. 35 shows BE-146 binding to human CD137 by FACS.

FIG. 36 shows BE-146 binding to human CEA by FACS.

FIG. 37 shows that BE-146 has no off target binding by FACS.

FIG. 38A-38C demonstrates CEA×CD137 multispecific antibody BE-146 induces the IL-2 and IFN-γ release from human PBMCs. FIG. 38A is a schematic diagram of CD137 activation via co-stimulating huPBMCs with BE-146 and HEK293/OS8 cells in the presence of MKN45 cells. FIG. 38B-38C shows BE-146 could induce IL-2 (FIG. 38B) and IFN-γ (FIG. 38C) from human PBMCs. PBMCs from 2 donors were tested. Results were shown in mean±SD of duplicates.

FIG. 39A-B demonstrates CEA×CD137 multispecific antibody BE-146 induces the IL-2 and IFN-γ release from human T cells. FIG. 39A shows BE-146 could induce IL-2 and IFN-γ (FIG. 39B) from human PBMCs. PBMCs from 2 donors were tested. Results were shown in mean±SD of duplicates.

FIG. 40A-40B demonstrates CEA×CD137 induced response is CEA dependent. FIG. 40A shows that BE-146 could induce significant IL-2 and IFN-γ release (FIG. 40B) from PBMCs against CEA over-expressing HEK293 cells, but not against HEK293 cells without CEA transduction. PBMCs from 3 donors were tested. Results were shown in mean SD of duplicates.

FIG. 41A-41B shows the CEA×CD137 induced response is not significantly blocked by recombinant soluble CEA. The results show that BE-146 induced IL-2 (FIG. 41A) and IFN-γ (FIG. 41B) release from PBMCs were not significantly blocked by 50 ng/ml or 500 ng/ml soluble CEA. PBMCs from 2 donors were tested. Results were shown in mean±SD of duplicates.

FIG. 42 demonstrates BE-146 enhances T cell activation using a cell based bioluminescent assay.

FIG. 43A-43B shows BE-146 enhances IFN-γ and IL-2 release from PBMCs against MKN45 (CEA^{high}) (FIG. 43A), but not NCI-N87 (CEA⁺) (FIG. 43B).

FIG. 44 demonstrates BE-146 dose-dependently enhances cytotoxicity of PBMCs against MKN45 cells.

FIG. 45 shows combination of BE-146 and BGB-A317 promotes IFN-γ secretion from PBMCs.

FIG. 46A is ELISA based FcγRs binding analysis of BE-146. FIG. 46B is ELISA based C1q binding activity of BE-146.

FIG. 47 shows the effect of BE-146 on tumor growth in the MC38/hCEA syngeneic model in humanized CD137 knock-in mice.

FIG. 48 shows the effect of BE-146 and Ch15mt on tumor growth in the CT26/hCEA syngeneic model in humanized CD137 knock-in mice.

FIG. 49 shows the effect of BE-146 and Ch15mt on tumor growth in the B16 F10/hCEA syngeneic model in humanized CD137 knock-in mice.

FIG. 50 shows the effect of BE-146 and Ch15mt on animal survival rate in the B16 F10/hCEA syngeneic model in humanized CD137 knock-in mice.

FIG. 51 shows that BE-146 does not have liver toxicity in vivo. High-dose Urelumab analog, but not BE-146, induced significantly increased alanine transaminase (ALT) and aspartate aminotransferase (AST) concentrations, and increased inflammatory cells infiltration in liver.

### DEFINITIONS

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The term “Carcinoembryonic antigen” or “CEA” refers to an approximately 70-100 kDa glycoprotein. CEA is also known as CEACAM5 or CD66e. The amino acid sequence of human CEA, (SEQ ID NO: 88) can also be found at accession number P06731 or NM_004363.2.

The term “CD137” or “TNFRSF9,” “ILA” or “41BB” refers to the amino acid sequence of human CD137, (SEQ ID NO: 135) can also be found at accession number Q07011 (TNR9_HUMAN) or U03397. The nucleic acid sequence of CD137 is set forth in SEQ ID NO:136.

The terms “administration,” “administering,” “treating,” and “treatment” as used herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “subject” in the context of the present disclosure is a mammal, e.g., a primate, preferably a higher primate, e.g., a human (e.g., a patient comprising, or at risk of having, a disorder described herein).

The term “affinity” as used herein refers to the strength of interaction between antibody and antigen. Within the antigen, the variable regions of the antibody interacts through non-covalent forces with the antigen at numerous sites. In general, the more interactions, the stronger the affinity.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL or Vκ) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four framework regions (FRs) arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The term “antibody” includes, but is not limited to, monoclonal antibodies, human antibodies, humanized antibodies, chimeric antibodies, and anti-idiotypic (anti-Id) antibodies. The antibodies can be of any isotype/class (e.g., IgG, IgE, IgM, IgD, IgA and IgY), or subclass (e.g., IgG1, IgG2, IgG3, IgG4, IgA1 and IgA2).

In some embodiments, the anti-CEA antibodies comprise at least one antigen-binding site, at least a variable region. In some embodiments, the anti-CEA antibodies comprise an antigen-binding fragment from an CEA antibody described herein. In some embodiments, the anti-CEA antibody is isolated or recombinant.

In some embodiments, the anti-CD137 antibodies comprise at least one antigen-binding site, at least a variable region. In some embodiments, the anti-CD137 antibodies comprise an antigen-binding fragment from an CD137 antibody described herein. In some embodiments, the anti-CD137 antibody is isolated or recombinant.

The term “monoclonal antibody” or “mAb” or “Mab” herein means a population of substantially homogeneous antibodies, i.e., the antibody molecules comprised in the population are identical in amino acid sequence except for possible naturally occurring mutations that can be present in minor amounts. In contrast, conventional (polyclonal) antibody preparations typically include a multitude of different antibodies having different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art. See, for example Kohler et al., Nature 1975 256:495-497; U.S. Pat. No. 4,376,110; Ausubel et al., CURRENT PROTOCOLS IN MOLECULAR BIOLOGY 1992; Harlow et al., ANTIBODIES: A LABORATORY MANUAL, Cold spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain can define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same in primary sequence.

Typically, the variable domains of both the heavy and light chains comprise three hypervariable regions, also called “complementarity determining regions (CDRs),” which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (or FR3), CDR-3 (CDR3), and FR-4 (or FR4). The positions of the CDRs and framework regions can be determined using various well known definitions in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); A1-Lazikani et al., J. Mol. Biol., 273:927-748 (1997) ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGap Align.

The term “hypervariable region” means the amino acid residues of an antibody that are responsible for antigen-binding. The hypervariable region comprises amino acid residues from a “CDR” (e.g., LCDR1, LCDR2 and LCDR3 in the light chain variable domain and HCDR1, HCDR2 and HCDR3 in the heavy chain variable domain). See, Kabat et al., (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues means those variable domain residues other than the hypervariable region residues defined herein as CDR residues.

Unless otherwise indicated, an “antigen-binding fragment” means antigen-binding fragments of antibodies, i.e. antibody fragments that retain the ability to bind specifically to the antigen bound by the full-length antibody, e.g., fragments that retain one or more CDR regions. Examples of antigen-binding fragments include, but not limited to, Fab, Fab′, F(ab′)2, and Fv fragments; diabodies; linear antibodies; single-chain antibody molecules, e.g., single chain Fv (ScFv); nanobodies and multispecific antibodies formed from antibody fragments.

As used herein, an antibody “specifically binds” to a target protein, meaning the antibody exhibits preferential binding to that target as compared to other proteins, but this specificity does not require absolute binding specificity. An antibody “specifically binds” or “selectively binds,” is used in the context of describing the interaction between an antigen (e.g., a protein) and an antibody, or antigen binding antibody fragment, refers to a binding reaction that is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times when compared to the background level and do not specifically bind in a significant amount to other antigens present in the sample. In one aspect, under designated immunoassay conditions, the antibody or antigen-binding fragment thereof, specifically bind to a particular antigen at least ten (10) times when compared to the background level of binding and does not specifically bind in a significant amount to other antigens present in the sample.

“Antigen-binding domain” as used herein, comprise at least three CDRs and specifically bind to an epitope. An “antigen-binding domain” of a multispecific antibody (e.g., a bispecific antibody) comprises a first antigen binding domain that specifically binds to a first epitope and a second antigen binding domain also comprised of at least three CDRs specifically binds to a second epitope. Multispecific antibodies can be bispecific, trispecific, tetraspecific etc., with antigen binding domains directed to each specific epitope. Multispecific antibodies can be multivalent (e.g., a bispecific tetravalent antibody) that comprises multiple antigen binding domains, for example, 2, 3, 4 or more antigen binding domains that specifically bind to a first epitope and 2, 3, 4 or more antigen binding domains that specifically bind a second epitope.

The term “human antibody” herein means an antibody that comprises human immunoglobulin protein sequences only. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

The term “equilibrium dissociation constant (K_{D}, M)” refers to the dissociation rate constant (kd, time⁻¹) divided by the association rate constant (ka, time⁻¹, M⁻¹). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M or 10⁻¹³ M.

The terms “cancer” or “tumor” herein has the broadest meaning as understood in the art and refers to the physiological condition in mammals that is typically characterized by unregulated cell growth. In the context of the present disclosure, the cancer is not limited to certain type or location.

In the context of the present disclosure, when reference is made to an amino acid sequence, the term “conservative substitution” means substitution of the original amino acid by a new amino acid that does not substantially alter the chemical, physical and/or functional properties of the antibody or fragment, e.g., its binding affinity to CEA or to CD137. Specifically, common conservative substations of amino acids are well known in the art.

The term “knob-into-hole” technology as used herein refers to amino acids that direct the pairing of two polypeptides together either in vitro or in vivo by introducing a spatial protuberance (knob) into one polypeptide and a socket or cavity (hole) into the other polypeptide at an interface in which they interact. For example, knob-into-holes have been introduced in the Fc:Fc binding interfaces, C_{L}:C_{H}I interfaces or VH/VL interfaces of antibodies (see, e.g., US 2011/0287009, US2007/0178552, WO 96/027011, WO 98/050431, and Zhu et al, 1997, Protein Science 6:781-788). In some embodiments, knob-into-holes insure the correct pairing of two different heavy chains together during the manufacture of multispecific antibodies. For example, multispecific antibodies having knob-into-hole amino acids in their Fc regions can further comprise single variable domains linked to each Fc region, or further comprise different heavy chain variable domains that pair with similar or different light chain variable domains. Knob-into-hole technology can also be used in the VH or VL regions to also insure correct pairing.

The term “knob” as used herein in the context of “knob-into-hole” technology refers to an amino acid change that introduces a protuberance (knob) into a polypeptide at an interface in which the polypeptide interacts with another polypeptide. In some embodiments, the other polypeptide has a hole mutation.

The term “hole” as used herein in the context of “knob-into-hole” refers to an amino acid change that introduces a socket or cavity (hole) into a polypeptide at an interface in which the polypeptide interacts with another polypeptide. In some embodiments, the other polypeptide has a knob mutation.

Examples of algorithms that are suitable for determining percent sequence identity and sequence similarity are the BLAST algorithms, which are described in Altschul et al, Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89: 10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4: 11-17, (1988), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some aspects, the present disclosure provides compositions, e.g., pharmaceutically acceptable compositions, which include anti-CEA×CD137 multispecific antibodies as described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g. by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

The term “therapeutically effective amount” as herein used, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The term “combination therapy” refers to the administration of two or more therapeutic agents to treat a therapeutic condition or disorder described in the present disclosure. Such administration encompasses co-administration of these therapeutic agents in a substantially simultaneous manner. Such administration also encompasses co-administration in multiple, or in separate containers (e.g., capsules, powders, and liquids) for each active ingredient. Powders and/or liquids can be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an anti-CEA×CD137 multispecific antibody is administered to the subject at the same time as, just before, or just after administration of an additional therapeutic agent. In certain embodiments, an anti-CEA×CD137 multispecific antibody is administered as a co-formulation with an additional therapeutic agent.

### DETAILED DESCRIPTION

The present disclosure provides for antibodies, antigen-binding fragments, and anti-CEA×CD137 multispecific antibodies. Furthermore, the present disclosure provides antibodies that have desirable pharmacokinetic characteristics and other desirable attributes, and thus can be used for reducing the likelihood of or treating cancer. The present disclosure further provides pharmaceutical compositions comprising the antibodies and methods of making and using such pharmaceutical compositions for the prevention and treatment of cancer and associated disorders.

### Anti-CEA Antibodies

The present disclosure provides for antibodies or antigen-binding fragments thereof that specifically bind to CEA. Antibodies or antigen-binding fragments of the present disclosure include, but are not limited to, the antibodies or antigen-binding fragments thereof, generated as described, below.

The present disclosure provides antibodies or antigen-binding fragments that specifically bind to CEA, wherein said antibodies or antibody fragments (e.g., antigen-binding fragments) comprise a VH domain having an amino acid sequence of SEQ ID NOs:14, 31 or 48 (Table 1). The present disclosure also provides antibodies or antigen-binding fragments that specifically bind CEA, wherein said antibodies or antigen-binding fragments comprise a HCDR having an amino acid sequence of any one of the HCDRs listed in Table 1. In one aspect, the present disclosure provides antibodies or antigen-binding fragments that specifically bind to CEA, wherein said antibodies comprise (or alternatively, consist of) one, two, three, or more HCDRs having an amino acid sequence of any of the HCDRs listed in Table 1.

The present disclosure provides for antibodies or antigen-binding fragments that specifically bind to CEA, wherein said antibodies or antigen-binding fragments comprise a VL domain having an amino acid sequence of SEQ ID NO:15, 32 or 49 (Table 1). The present disclosure also provides antibodies or antigen-binding fragments that specifically bind to CEA, wherein said antibodies or antigen-binding fragments comprise a LCDR having an amino acid sequence of any one of the LCDRs listed in Table 1. In particular, the disclosure provides for antibodies or antigen-binding fragments that specifically bind to CEA, said antibodies or antigen-binding fragments comprise (or alternatively, consist of) one, two, three or more LCDRs having an amino acid sequence of any of the LCDRs listed in Table 1.

Other antibodies or antigen-binding fragments thereof of the present disclosure include amino acids that have been changed, yet have at least 60%, 70%, 80%, 90%, 95% or 99% percent identity in the CDR regions with the CDR regions disclosed in Table 1. In some aspects, it includes amino acid changes wherein no more than 1, 2, 3, 4 or 5 amino acids have been changed in the CDR regions when compared with the CDR regions depicted in the sequence described in Table 1.

Other antibodies of the present disclosure include those where the amino acids or nucleic acids encoding the amino acids have been changed; yet have at least 60%, 70%, 80%, 90%, 95% or 99% percent identity to the sequences described in Table 1. In some aspects, it includes changes in the amino acid sequences wherein no more than 1, 2, 3, 4 or 5 amino acids have been changed in the variable regions when compared with the variable regions depicted in the sequence described in Table 1, while retaining substantially the same therapeutic activity.

The present disclosure also provides nucleic acid sequences that encode VH, VL, the full length heavy chain, and the full length light chain of the antibodies that specifically bind to CEA. Such nucleic acid sequences can be optimized for expression in mammalian cells.

TABLE 1
Amino acid and nucleic acid sequences

| ANTIBODY | SEQ ID NO | | SEQUENCE |
|---|---|---|---|
| BGA113K | SEQ ID NO: | HCDR1 | GYIFTSYY |
| (IMGT) | 1 | (IMGT) | |
| | SEQ ID NO: | HCDR2 | INPQTGKT |
| | 2 | (IMGT) | |
| | SEQ ID NO: | HCDR3 | AREYGNYNYPLDY |
| | 3 | (IMGT) | |
| | SEQ ID NO: | LCDR1 | ENQYGY |
| | 4 | (IMGT) | |
| | SEQ ID NO: | LCDR2 | NY |
| | 5 | (IMGT) | |
| | SEQ ID NO: | LCDR3 | QHHLGTPYT |
| | 6 | (IMGT) | |
| BGA113K | SEQ ID NO: | HCDR1 | SYYLH |
| (Kabat) | 7 | (Kabat) | |
| | SEQ ID NO: | HCDR2 | YINPQTGKTSYAQKFQG |
| | 8 | (Kabat) | |
| | SEQ ID NO: | HCDR3 | EYGNYNYPLDY |
| | 9 | (Kabat) | |
| | SEQ ID NO: | LCDR1 | RASENQYGYLA |
| | 10 | (Kabat) | |
| | SEQ ID NO: | LCDR2 | NYKNLVE |
| | 11 | (Kabat) | |
| | SEQ ID NO: | LCDR3 | QHHLGTPYT |
| | 6 | (Kabat) | |
| BGA113K | SEQ ID NO: | HCDR1 | GYIFTSY |
| (Chothia) | 12 | (Chothia) | |
| | SEQ ID NO: | HCDR2 | NPQTGK |
| | 13 | (Chothia) | |
| | SEQ ID NO: | HCDR3 | EYGNYNYPLDY |
| | 9 | (Chothia) | |
| | SEQ ID NO: | LCDR1 | RASENQYGYLA |
| | 10 | (Chothia) | |
| | SEQ ID NO: | LCDR2 | NYKNLVE |
| | 11 | (Chothia) | |
| | SEQ ID NO: | LCDR3 | QHHLGTPYT |
| | 6 | (Chothia) | |
| BGA113K | SEQ ID NO: | VH | QVQLVQSGAEVKKPGASVKVSCKASGYIF |
| | 14 | | TSYYLHWVRQAPGQGLEWIGYINPQTGK |
| | | | TSYAQKFQGRVTMTRDTSTSTVYMELSSL |
| | | | RSEDTAVYYCAREYGNYNYPLDYWGQGT |
| | | | LVTVSS |
| | SEQ ID NO: | VL | DIQMTQSPSSLSASVGDRVTITCRASENQY |
| | 15 | | GYLAWYQQKPGKVPKLLIYNYKNLVEGV |
| | | | PSRFSGSGSGTDFTLTISSLQPEDVATYYCQ |
| | | | HHLGTPYTFGQGTKVEIK |
| | SEQ ID NO: | VH DNA | CAGGTGCAGCTGGTGCAGAGCGGCGCG |
| | 16 | | GAAGTGAAAAAACCGGGCGCGAGCGTG |
| | | | AAAGTGAGCTGCAAAGCGAGCGGCTATA |
| | | | TTTTTACCAGCTATTACCTGCATTGGGTG |
| | | | CGCCAGGCGCCGGGCCAGGGCCTGGAAT |
| | | | GGATTGGCTATATTAACCCGCAGACCGGC |
| | | | AAGACCAGCTATGCCCAGAAATTTCAGG |
| | | | GCCGCGTGACCATGACCCGCGATACCAG |
| | | | CACCAGCACCGTGTATATGGAACTGAGC |
| | | | AGCCTGCGCAGCGAAGATACCGCGGTGT |
| | | | ATTATTGCGCGCGCGAATATGGCAACTAT |
| | | | AACTATCCGCTGGATTATTGGGGCCAGGG |
| | | | CACCCTGGTGACCGTGAGCAGC |
| | SEQ ID NO: | VL DNA | GATATTCAGATGACCCAGAGCCCGAGCA |
| | 17 | | GCCTGAGCGCGAGCGTGGGCGATCGCGT |
| | | | GACCATTACCTGCCGCGCGAGCGAAAAC |
| | | | CAGTATGGCTATCTGGCGTGGTATCAGCA |
| | | | GAAACCGGGCAAAGTGCCGAAACTGCT |
| | | | GATTTATAACTATAAAAACCTGGTGGAAG |
| | | | GCGTGCCGAGCCGCTTTAGCGGCAGCGG |
| | | | CAGCGGCACCGATTTTACCCTGACCATTA |
| | | | GCAGCCTGCAGCCGGAAGATGTGGCGAC |
| | | | CTATTATTGCCAGCATCATCTGGGCACCC |
| | | | CGTATACCTTTGGCCAGGGCACCAAAGT |
| | | | GGAAATTAAA |
| BGA190 | SEQ ID NO: | HCDR1 | GFTFSSFG |
| (IMGT) | 18 | (IMGT) | |
| | SEQ ID NO: | HCDR2 | ISIGSDII |
| | 19 | (IMGT) | |
| | SEQ ID NO: | HCDR3 | TRRSYRSYWYFDV |
| | 20 | (IMGT) | |
| | SEQ ID NO: | LCDR1 | ENIYSY |
| | 21 | (IMGT) | |
| | SEQ ID NO: | LCDR2 | NA |
| | 22 | (IMGT) | |
| | SEQ ID NO: | LCDR3 | QHHFLSPWM |
| | 23 | (IMGT) | |
| BGA190 | SEQ ID NO: | HCDR1 | SFGMH |
| (Kabat) | 24 | (Kabat) | |
| | SEQ ID NO: | HCDR2 | YISIGSDIIYYADTVKG |
| | 25 | (Kabat) | |
| | SEQ ID NO: | HCDR3 | RSYRSYWYFDV |
| | 26 | (Kabat) | |
| | SEQ ID NO: | LCDR1 | RTSENIYSYLA |
| | 27 | (Kabat) | |
| | SEQ ID NO: | LCDR2 | NAKTLAE |
| | 28 | (Kabat) | |
| | SEQ ID NO: | LCDR3 | QHHFLSPWM |
| | 23 | (Kabat) | |
| BGA190 | SEQ ID NO: | HCDR1 | GFTFSSF |
| (Chothia) | 29 | (Chothia) | |
| | SEQ ID NO: | HCDR2 | SIGSDI |
| | 30 | (Chothia) | |
| | SEQ ID NO: | HCDR3 | RSYRSYWYFDV |
| | 26 | (Chothia) | |
| | SEQ ID NO: | LCDR1 | RTSENIYSYLA |
| | 27 | (Chothia) | |
| | SEQ ID NO: | LCDR2 | NAKTLAE |
| | 28 | (Chothia) | |
| | SEQ ID NO: | LCDR3 | QHHFLSPWM |
| | 23 | (Chothia) | |
| BGA190 | SEQ ID NO: | VH | DVQLVESGGGLVQPGGSRELSCAASGFTF |
| | 31 | | SSFGMHWVRQAPERGLEWVAYISIGSDIIY |
| | | | YADTVKGRFTISRDNPKNTLFLQMTSLRSE |
| | | | DTAVYYCTRRSYRSYWYFDVWGAGTTVT |
| | | | VSS |
| | SEQ ID NO: | VL | DIQMTQSPASLSASVGETVTITCRTSENIYS |
| | 32 | | YLAWYQQKQGKSPHLLVYNAKTLAEGVP |
| | | | SRFSGSGSGTQFSLKIISLQPEDFGTYYCQH |
| | | | HFLSPWMFGGGTKLEIK |
| | SEQ ID NO: | VH DNA | GATGTGCAGCTGGTGGAGTCTGGGGGAG |
| | 33 | | GCTTAGTGCAGCCTGGAGGGTCCCGGGA |
| | | | ACTCTCCTGTGCAGCCTCTGGATTCACTT |
| | | | TCAGTAGCTTTGGAATGCACTGGGTTCGT |
| | | | CAGGCTCCAGAGAGGGGGCTGGAGTGG |
| | | | GTCGCATACATTAGTATTGGCAGTGATAT |
| | | | CATCTACTATGCAGACACAGTGAAGGGC |
| | | | CGATTCACCATCTCCAGAGACAATCCCA |
| | | | AGAACACCCTGTTCCTGCAAATGACCAG |
| | | | TCTGAGGTCTGAGGACACGGCCGTGTAT |
| | | | TACTGTACAAGAAGGTCTTATAGGTCCTA |
| | | | CTGGTACTTCGATGTCTGGGGCGCAGGG |
| | | | ACCACGGTCACCGTCTCCTCA |
| | SEQ ID NO: | VL DNA | GACATCCAGATGACTCAGTCTCCAGCCT |
| | 34 | | CCCTATCTGCATCTGTGGGAGAAACTGTC |
| | | | ACCATCACATGTCGAACAAGTGAGAATA |
| | | | TTTACAGTTATTTAGCATGGTATCAGCAG |
| | | | AAACAGGGAAAATCTCCTCATCTCCTGG |
| | | | TCTATAATGCAAAAACCTTAGCAGAGGG |
| | | | TGTGCCATCAAGGTTCAGTGGCAGTGGA |
| | | | TCAGGCACACAGTTTTCTCTGAAGATCAT |
| | | | CAGCCTGCAGCCTGAAGATTTTGGGACT |
| | | | TATTACTGTCAGCATCATTTTCTTAGTCCG |
| | | | TGGATGTTCGGTGGAGGCACCAAGCTGG |
| | | | AAATCAAA |
| BGA288 | SEQ ID NO: | HCDR1 | GYTFTDYN |
| (IMGT) | 35 | (IMGT) | |
| | SEQ ID NO: | HCDR2 | IGPNYGGT |
| | 36 | (IMGT) | |
| | SEQ ID NO: | HCDR3 | ARRGSIPRAVDY |
| | 37 | (IMGT) | |
| | SEQ ID NO: | LCDR1 | QDIHNY |
| | 38 | (IMGT) | |
| | SEQ ID NO: | LCDR2 | RA |
| | 39 | (IMGT) | |
| | SEQ ID NO: | LCDR3 | LQYDEFPYT |
| | 40 | (IMGT) | |
| BGA288 | SEQ ID NO: | HCDR1 | DYNID |
| (Kabat) | 41 | (Kabat) | |
| | SEQ ID NO: | HCDR2 | DIGPNYGGTIYNQKFKG |
| | 42 | (Kabat) | |
| | SEQ ID NO: | HCDR3 | RGSIPRAVDY |
| | 43 | (Kabat) | |
| | SEQ ID NO: | LCDRI | KASQDIHNYLS |
| | 44 | (Kabat) | |
| | SEQ ID NO: | LCDR2 | RANRLVD |
| | 45 | (Kabat) | |
| | SEQ ID NO: | LCDR3 | LQYDEFPYT |
| | 40 | (Kabat) | |
| BGA288 | SEQ ID NO: | HCDR1 | GYTFTDY |
| (Chothia) | 46 | (Chothia) | |
| | SEQ ID NO: | HCDR2 | GPNYGG |
| | 47 | (Chothia) | |
| | SEQ ID NO: | HCDR3 | RGSIPRAVDY |
| | 43 | (Chothia) | |
| | SEQ ID NO: | LCDR1 | KASQDIHNYLS |
| | 44 | (Chothia) | |
| | SEQ ID NO: | LCDR2 | RANRLVD |
| | 45 | (Chothia) | |
| | SEQ ID NO: | LCDR3 | LQYDEFPYT |
| | 40 | (Chothia) | |
| BGA288 | SEQ ID NO: | VH | EVLLQQSGPELVKPGASVKIFCKASGYTFT |
| | 48 | | DYNIDWVQQSHGKSLEWIGDIGPNYGGTI |
| | | | YNQKFKGKATLTVAKSSSTAYMELRSLTSE |
| | | | DTAVYYCARRGSIPRAVDYWGQGTSVTVS |
| | | | S |
| | SEQ ID NO: | VL | DIKMTQSPSSMYASLGERVTITCKASQDIH |
| | 49 | | NYLSWFQQKPGKSPKTLIYRANRLVDGVP |
| | | | SRFSGSGSGQDYSLTISSLEYEDMGIYYCL |
| | | | QYDEFPYTFGGGTKLEIK |
| | SEQ ID NO: | VH DNA | GAGGTCCTGCTGCAACAGTCTGGACCTG |
| | 50 | | AGCTGGTGAAGCCTGGGGCTTCAGTGAA |
| | | | GATTTTCTGTAAGGCTTCTGGATACACAT |
| | | | TCACTGACTACAACATAGACTGGGTGCA |
| | | | GCAGAGCCATGGAAAGAGCCTTGAGTG |
| | | | GATTGGAGATATTGGTCCTAATTATGGTG |
| | | | GTACTATCTACAACCAGAAGTTCAAGGG |
| | | | CAAGGCCACATTGACTGTAGCCAAGTCC |
| | | | TCCAGCACAGCCTACATGGAGCTCCGCA |
| | | | GCCTGACATCTGAGGACACTGCAGTCTA |
| | | | TTACTGTGCAAGACGCGGTAGCATCCCG |
| | | | AGGGCTGTGGACTACTGGGGTCAAGGAA |
| | | | CCTCAGTCACCGTCTCCTCA |
| | SEQ ID NO: | VL DNA | GACATCAAGATGACCCAGTCTCCATCTTC |
| | 51 | | CATGTATGCATCTCTAGGAGAGAGAGTC |
| | | | ACTATCACTTGCAAGGCGAGTCAGGACA |
| | | | TTCATAACTATTTAAGCTGGTTCCAGCAG |
| | | | AAACCAGGGAAATCTCCTAAGACCCTGA |
| | | | TCTATCGTGCAAACAGATTGGTAGATGGG |
| | | | GTCCCATCAAGGTTCAGTGGCAGTGGAT |
| | | | CTGGGCAAGATTATTCTCTCACCATCAGC |
| | | | AGCCTGGAGTATGAAGATATGGGAATTTA |
| | | | TTATTGTCTACAGTATGATGAGTTTCCGTA |
| | | | CACGTTCGGAGGGGGGACCAAGCTGGA |
| | | | AATAAAA |

The present disclosure provides antibodies and antigen-binding fragments thereof that bind to an epitope of human CEA. In certain aspects the antibodies and antigen-binding fragments can bind to the same epitope of CEA.

The present disclosure also provides for antibodies and antigen-binding fragments thereof that bind to the same epitope as do the anti-CEA antibodies described in Table 1. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of the present disclosure to CEA demonstrates that the test antibody can compete with that antibody or antigen-binding fragments thereof for binding to CEA. Such an antibody can, without being bound to any one theory, bind to the same or a related (e.g., a structurally similar or spatially proximal) epitope on CEA as the antibody or antigen-binding fragments thereof with which it competes. In a certain aspect, the antibody that binds to the same epitope on CEA as the antibodies or antigen-binding fragments thereof of the present disclosure is a human or humanized monoclonal antibody. Such human or humanized monoclonal antibodies can be prepared and isolated as described herein.

### Anti-CD137 Antibodies

TABLE 2
Sequences

| Antibody | SEQ ID NO | | SEQUENCE |
|---|---|---|---|
| BGA-7207 | SEQ ID NO: 52 | HCDR1 (IMGT) | GFRLDTTE |
| (IMGT) | SEQ ID NO: 53 | HCDR2 (IMGT) | IMGISGST |
| | SEQ ID NO: 54 | HCDR3 (IMGT) | ARVVDSLFDDSAVFDY |
| BGA-7207 | SEQ ID NO: 55 | HCDR1 (Kabat) | TTEVG |
| (Kabat) | SEQ ID NO: 56 | HCDR2 (Kabat) | TIMGISGSTYYADSVKG |
| | SEQ ID NO: 57 | HCDR3 (Kabat) | VVDSLFDDSAVFDY |
| BGA-7207 | SEQ ID NO: 58 | HCDR1 (Chothia) | GFRLDTT |
| (Chothia) | SEQ ID NO: 59 | HCDR2 (Chothia) | MGISGS |
| | SEQ ID NO: 57 | HCDR3 (Chothia) | VVDSLFDDSAVFDY |
| BGA-7207 | SEQ ID NO: 60 | VH | EVQLLESGGGLVQPGGSLRLSCAASGFR |
| | | | LDTTEVGWVRQAPGKGLEWVSTIMGIS |
| | | | GSTYYADSVKGRFTISRDNSKNTLYLQ |
| | | | MNSLRAEDTAVYYCARVVDSLFDDSAV |
| | | | FDYWGQGTLVTVSS |
| | SEQ ID NO: 61 | VH DNA | GAGGTCCAGTTACTTGAGAGTGGTGG |
| | | | AGGTCTGGTCCAACCAGGAGGTTCGC |
| | | | TGCGTTTATCCTGCGCCGCGTCTGGAT |
| | | | TCAGATTGGACACCACCGAAGTAGGT |
| | | | TGGGTGCGTCAAGCGCCGGGGAAAGG |
| | | | ACTGGAATGGGTCTCCACCATCATGGG |
| | | | TATTAGTGGTTCGACATACTATGCGGA |
| | | | CAGTGTCAAAGGGCGCTTTACGATCTC |
| | | | GCGCGATAACTCAAAAAATACTCTTTA |
| | | | CCTTCAAATGAATAGCCTTCGTGCTGA |
| | | | GGACACTGCGGTGTACTACTGCGCCC |
| | | | GCGTCGTCGATTCCCTCTTTGATGACA |
| | | | GCGCCGTTTTTGATTACTGGGGACAGG |
| | | | GCACCTTAGTTACAGTCTCATCG |
| BGA-4712 | SEQ ID NO: 62 | HCDR1 (IMGT) | GFMLSAED |
| (IMGT) | SEQ ID NO: 63 | HCDR2 (IMGT) | ILDFGGST |
| | SEQ ID NO: 64 | HCDR3 (IMGT) | ARVVYHAGGGVTFDY |
| BGA-4712 | SEQ ID NO: 65 | HCDR1 (Kabat) | AEDVG |
| (Kabat) | SEQ ID NO: 66 | HCDR2 (Kabat) | AILDFGGSTYYADSVKG |
| | SEQ ID NO: 67 | HCDR3 (Kabat) | VVYHAGGGVTFDY |
| BGA-4712 | SEQ ID NO: 68 | HCDR1 (Chothia) | GFMLSAE |
| (Chothia) | SEQ ID NO: 69 | HCDR2 (Chothia) | LDFGGS |
| | SEQ ID NO: 67 | HCDR3 (Chothia) | VVYHAGGGVTFDY |
| BGA-4712 | SEQ ID NO: 70 | VH | EVQLLESGGGLVQPGGSLRLSCAASGF |
| | | | MLSAEDVGWVRQAPGKGLEWVSAILD |
| | | | FGGSTYYADSVKGRFTISRDNSKNTLYL |
| | | | QMNSLRAEDTAVYYCARVVYHAGGGV |
| | | | TFDYWGQGTLVTVSS |
| | SEQ ID NO: 71 | VH DNA | GAGGTCCAGTTACTTGAGAGTGGTGG |
| | | | AGGTCTGGTCCAACCAGGAGGTTCGC |
| | | | TGCGTTTATCCTGCGCCGCGTCTGGAT |
| | | | TCATGTTGTCCGCCGAAGACGTGGGTT |
| | | | GGGTGCGTCAAGCGCCGGGGAAAGG |
| | | | ACTGGAATGGGTCTCCGCCATCTTGGA |
| | | | TTTTGGTGGTTCGACATACTATGCGGA |
| | | | CAGTGTCAAAGGGCGCTTTACGATCTC |
| | | | GCGCGATAACTCAAAAAATACTCTTTA |
| | | | CCTTCAAATGAATAGCCTTCGTGCTGA |
| | | | GGACACTGCGGTGTACTACTGCGCCC |
| | | | GCGTCGTCTACCATGCTGGTGGTGGCG |
| | | | TCACCTTTGATTACTGGGGACAGGGCA |
| | | | CCTTAGTTACAGTCTCATCG |
| BGA-4712-M3 | SEQ ID NO: 72 | HCDR1 (IMGT) | GFTLSAED |
| (IMGT) | SEQ ID NO: 63 | HCDR2 (IMGT) | ILDFGGST |
| | SEQ ID NO: 64 | HCDR3 (IMGT) | ARVVYHAGGGVTFDY |
| BGA-4712-M3 | SEQ ID NO: 65 | HCDR1 (Kabat) | AEDVG |
| (Kabat) | SEQ ID NO: 73 | HCDR2 (Kabat) | AILDFGGSTYYAESVKG |
| | SEQ ID NO: 67 | HCDR3 (Kabat) | VVYHAGGGVTFDY |
| BGA-4712-M3 | SEQ ID NO: 74 | HCDR1 (Chothia) | GFTLSAE |
| (Chothia) | SEQ ID NO: 69 | HCDR2 (Chothia) | LDFGGS |
| | SEQ ID NO: 67 | HCDR3 (Chothia) | VVYHAGGGVTFDY |
| BGA-4712-M3 | SEQ ID NO: 75 | VH | EVQLLESGGGLVQPGGSLRLSCAASGFT |
| | | | LSAEDVGWVRQAPGKGLEWVSAILDF |
| | | | GGSTYYAESVKGRFTISRDNAKNTLYLQ |
| | | | MSSLRAEDTAVYYCARVVYHAGGGVT |
| | | | FDYWGQGTLVTVSS |
| | SEQ ID NO: 76 | VH DNA | GAGGTCCAGTTACTTGAGAGTGGTGG |
| | | | AGGTCTGGTCCAACCAGGAGGTTCGC |
| | | | TGCGTTTATCCTGCGCCGCGTCTGGAT |
| | | | TCACGTTGTCCGCCGAAGACGTGGGT |
| | | | TGGGTGCGTCAAGCGCCGGGGAAAGG |
| | | | ACTGGAATGGGTCTCCGCCATCTTGGA |
| | | | TTTTGGTGGTTCGACATACTATGCGGA |
| | | | AAGTGTCAAAGGGCGCTTTACGATCTC |
| | | | GCGCGATAACGCAAAAAATACTCTTTA |
| | | | CCTTCAAATGTCTAGCCTTCGTGCTGA |
| | | | GGACACTGCGGTGTACTACTGCGCCC |
| | | | GCGTCGTCTACCATGCTGGTGGTGGCG |
| | | | TCACCTTTGATTACTGGGGACAGGGCA |
| | | | CCTTAGTTACAGTCTCATCG |
| BGA-5623 | SEQ ID NO: 77 | HCDR1 (IMGT) | GFTVSAED |
| (IMGT) | SEQ ID NO: 78 | HCDR2 (IMGT) | ILDKGGST |
| | SEQ ID NO: 79 | HCDR3 (IMGT) | ARIVYHAGGGVTFDT |
| BGA-5623 | SEQ ID NO: 65 | HCDR1 (Kabat) | AEDVG |
| (Kabat) | SEQ ID NO: 80 | HCDR2 (Kabat) | AILDKGGSTYYAESVKG |
| | SEQ ID NO: 81 | HCDR3 (Kabat) | IVYHAGGGVTFDT |
| BGA-5623 | SEQ ID NO: 82 | HCDR1 (Chothia) | GFTVSAE |
| (Chothia) | SEQ ID NO: 83 | HCDR2 (Chothia) | LDKGGS |
| | SEQ ID NO: 81 | HCDR3 (Chothia) | IVYHAGGGVTFDT |
| BGA-5623 | SEQ ID NO: 84 | VH | EVQLLESGGGLVQPGGSLRLSCAASGFT |
| | | | VSAEDVGWVRQAPGKGLEWVSAILDK |
| | | | GGSTYYAESVKGRFTISRDNAKNTLYLQ |
| | | | MSSLRAEDTAVYYCARIVYHAGGGVTF |
| | | | DTRGQGTQVTVSS |
| | SEQ ID NO: 85 | VH DNA | GAGGTCCAGTTACTTGAGAGTGGTGG |
| | | | AGGTCTGGTCCAACCAGGAGGTTCGC |
| | | | TGCGTTTATCCTGCGCCGCGTCTGGAT |
| | | | TCACGGTTTCCGCCGAAGACGTGGGT |
| | | | TGGGTGCGTCAAGCGCCGGGGAAAGG |
| | | | ACTGGAATGGGTCTCCGCCATCTTGGA |
| | | | TAAGGGTGGTTCGACATACTATGCGGA |
| | | | AAGTGTCAAAGGGCGCTTTACGATCTC |
| | | | GCGCGATAACGCAAAAAATACTCTTTA |
| | | | CCTTCAAATGTCTAGCCTTCGTGCTGA |
| | | | GGACACTGCGGTGTACTACTGCGCCC |
| | | | GCATTGTCTACCATGCTGGTGGTGGCG |
| | | | TCACCTTTGATACTCGGGGACAGGGC |
| | | | ACCCAAGTTACAGTCTCATCG |
| BGA-7556 | SEQ ID NO: 86 | VH | EVQLLESGGGLVQPGGSLRLSCAASGFT |
| | | | LSAEDVGWVRQAPGKGLEWVSAILDF |
| | | | GGSTYYAESVKGRFTISRDNAKNTLYLQ |
| | | | MSSLRAEDTAVYYCARVVYHAGGGVT |
| | | | FDYRGQGTQVTVSS |
| | SEQ ID NO: 87 | VH DNA | GAGGTCCAGTTACTTGAGAGTGGTGG |
| | | | AGGTCTGGTCCAACCAGGAGGTTCGC |
| | | | TGCGTTTATCCTGCGCCGCGTCTGGAT |
| | | | TCACGTTGTCCGCCGAAGACGTGGGT |
| | | | TGGGTGCGTCAAGCGCCGGGGAAAGG |
| | | | ACTGGAATGGGTCTCCGCCATCTTGGA |
| | | | TTTTGGTGGTTCGACATACTATGCGGA |
| | | | AAGTGTCAAAGGGCGCTTTACGATCTC |
| | | | GCGCGATAACGCAAAAAATACTCTTTA |
| | | | CCTTCAAATGTCTAGCCTTCGTGCTGA |
| | | | GGACACTGCGGTGTACTACTGCGCCC |
| | | | GCGTCGTCTACCATGCTGGTGGTGGCG |
| | | | TCACCTTTGATTACCGGGGACAGGGC |
| | | | ACCCAAGTTACAGTCTCATCG |

The present disclosure provides for antibodies or antigen-binding fragments thereof that specifically bind to CD3137. Antibodies or antigen-binding fragments of the present disclosure include, but are not limited to, the antibodies or antigen-binding fragments thereof, generated as described, below.

The present disclosure provides antibodies or antigen-binding fragments that specifically bind to CD137, wherein said antibodies or antibody fragments (e.g., antigen-binding fragments) comprise a VH domain having an amino acid sequence of SEQ ID NO:60, SEQ ID NO:70, SEQ ID NO:75, SEQ ID NO:84 or SEQ ID NO:86 (Table 2). The present disclosure also provides antibodies or antigen-binding fragments that specifically bind CD137, wherein said antibodies or antigen-binding fragments comprise a HCDR having an amino acid sequence of any one of the HCDRs listed in Table 2. In one aspect, the present disclosure provides antibodies or antigen-binding fragments that specifically bind to CD137, wherein said antibodies comprise (or alternatively, consist of) one, two, three, or more HCDRs having an amino acid sequence of any of the HCDRs listed in Table 2.

Other antibodies or antigen-binding fragments thereof of the present disclosure include amino acids that have been changed, yet have at least 60%, 70%, 80%, 90%, 95% or 99% percent identity in the CDR regions with the CDR regions disclosed in Table 2. In some aspects, it includes amino acid changes wherein no more than 1, 2, 3, 4 or 5 amino acids have been changed in the CDR regions when compared with the CDR regions depicted in the sequence described in Table 2.

Other antibodies of the present disclosure include those where the amino acids or nucleic acids encoding the amino acids have been changed; yet have at least 60%, 70%, 80%, 90%, 95% or 99% percent identity to the sequences described in Table 2. In some aspects, it includes changes in the amino acid sequences wherein no more than 1, 2, 3, 4 or 5 amino acids have been changed in the variable regions when compared with the variable regions depicted in the sequence described in Table 2, while retaining substantially the same therapeutic activity.

The present disclosure also provides nucleic acid sequences that encode VH, VL, the full length heavy chain, and the full length light chain of the antibodies that specifically bind to CD137. Such nucleic acid sequences can be optimized for expression in mammalian cells.

The present disclosure provides antibodies and antigen-binding fragments thereof that bind to an epitope of human CD137. In certain aspects the antibodies and antigen-binding fragments can bind to the same epitope of CD137.

The present disclosure also provides for antibodies and antigen-binding fragments thereof that bind to the same epitope as do the anti-CD137 antibodies described in Table 2. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of the present disclosure to CD137 demonstrates that the test antibody can compete with that antibody or antigen-binding fragments thereof for binding to CD137. Such an antibody can, without being bound to any one theory, bind to the same or a related (e.g., a structurally similar or spatially proximal) epitope on CD137 as the antibody or antigen-binding fragments thereof with which it competes. In a certain aspect, the antibody that binds to the same epitope on CD137 as the antibodies or antigen-binding fragments thereof of the present disclosure is a human or humanized monoclonal antibody. Such human or humanized monoclonal antibodies can be prepared and isolated as described herein.

### Anti-CEA×CD137 Multispecific Antibodies

In one embodiment, the anti-CEA and anti-CD137 antibodies as disclosed herein can be incorporated into an anti-CEA×CD137 multispecific antibody. An antibody molecule is a multispecific antibody molecule, for example, it comprises a number of antigen binding domains, wherein at least one antigen binding domain sequence specifically binds CEA as a first epitope and a second antigen binding domain sequence specifically binds CD137 as a second epitope. In one embodiment, the multispecific antibody comprises a third, fourth or fifth antigen binding domain. In one embodiment, the multispecific antibody is a bispecific antibody, a trispecific antibody, or tetraspecific antibody. In each example, the multispecific antibody comprises at least one anti-CEA antigen binding domain and at least one anti-CD137 antigen binding domain.

In one embodiment, the multispecific antibody is a bispecific antibody. As used herein, a bispecific antibody specifically binds only two antigens. The bispecific antibody comprises a first antigen binding domain which specifically binds CEA and a second antigen binding domain that specifically binds CD137. This includes a bispecific antibody comprising a heavy chain variable domain and a light chain variable domain which specifically bind CEA as a first epitope and a heavy chain variable domain which specifically bind CD137 as a second epitope. In another embodiment, the bispecific antibody comprises an antigen binding fragment of an antibody that specifically binds CEA and an antigen binding fragment that specially binds CD137. The bispecific antibody that comprises antigen binding fragments, the antigen-binding fragment can be a Fab, F(ab′)2, Fv, or a single chain Fv (ScFv) or a scFv.

Previous experimentation (Coloma and Morrison Nature Biotech. 15: 159-163 (1997)) described a tetravalent bispecific antibody which was engineered by fusing DNA encoding a single chain anti-dansyl antibody Fv (scFv) after the C terminus (CH3-scFv) or after the hinge (hinge-scFv) of an lgG3 anti-dansyl antibody. The present disclosure provides multivalent antibodies (e.g. tetravalent antibodies) with at least two antigen binding domains, which can be readily produced by recombinant expression of nucleic acid encoding the polypeptide chains of the antibody. The multivalent antibody herein comprises three to eight, but preferably four, antigen binding domains, which specifically bind at least two antigens.

### Linkers

It is also understood that the domains and/or regions of the polypeptide chains of the bispecific tetravalent antibody can be separated by linker regions of various lengths. In some embodiments, the antigen binding domains are separated from each other, a CL, CH1, hinge, CH2, CH3, or the entire Fc region by a linker region. For example, VL1-CL-(linker) VH2-CH1 Such linker region may comprise a random assortment of amino acids, or a restricted set of amino acids. Such linker regions can be flexible or rigid (see US2009/0155275).

Multispecific antibodies have been constructed by genetically fusing two single chain Fv (scFv) or Fab fragments with or without the use of flexible linkers (Mallender et al., J. Biol. Chem. 1994 269:199-206; Macket al., Proc. Natl. Acad. Sci. USA. 1995 92:7021-5; Zapata et al., Protein Eng. 1995 8.1057-62), via a dimerization device such as leucine Zipper (Kostelny et al., J. Immunol. 1992148:1547-53; de Kruifetal J. Biol. Chem. 1996 271:7630-4) and Ig C/CH1 domains (Muller et al., FEBS Lett. 422:259-64); by diabody (Holliger et al., (1993) Proc. Nat. Acad. Sci. USA. 1998 90:6444-8; Zhu et al., Bio/Technology (NY) 1996 14:192-6); Fab-scFv fusion (Schoonjans et al., J. Immunol. 2000 165:7050-7); and mini antibody formats (Packet al., Biochemistry 1992.31:1579-84; Packet al., Bio/Technology 1993 11:1271-7).

The bispecific tetravalent antibodies as disclosed herein comprise a linker region of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues between one or more of its antigen binding domains, CL domains, CH1 domains, Hinge region, CH2 domains, CH3 domains, or Fc regions. In some embodiments, the amino acids glycine and serine comprise the amino acids within the linker region. In another embodiment, the linker can be GS (SEQ ID NO: 317), GGS (SEQ ID NO: 318), GSG (SEQ ID NO: 319), SGG (SEQ ID NO: 320), GGG (SEQ ID NO: 321), GGGS (SEQ ID NO: 322), SGGG (SEQ ID NO: 323), GGGGS (SEQ ID NO: 324), GGGGSGS (SEQ ID NO: 325), GGGGSGS (SEQ ID NO: 326), GGGGSGGS (SEQ ID NO: 327), GGGGSGGGGS (SEQ ID NO: 328), GGGGSGGGGSGGGGS (SEQ ID NO: 329), AKTTPKLEEGEFSEAR (SEQ ID NO: 330), AKTTPKLEEGEFSEARV (SEQ ID NO: 331), AKTTPKLGG (SEQ ID NO: 332), SAKTTPKLGG (SEQ ID NO: 333), AKTTPKLEEGEFSEARV (SEQ ID NO: 334), SAKTTP (SEQ ID NO: 335), SAKTTPKLGG (SEQ ID NO: 336), RADAAP (SEQ ID NO: 337), RADAAPTVS (SEQ ID NO: 338), RADAAAAGGPGS (SEQ ID NO: 339), RADAAAA(G4S)4(SEQ ID NO: 340), SAKTTP (SEQ ID NO: 341), SAKTTPKLGG (SEQ ID NO: 342), SAKTTPKLEEGEFSEARV (SEQ ID NO: 343), ADAAP (SEQ ID NO: 344), ADAAPTVSIFPP (SEQ ID NO: 345), TVAAP (SEQ ID NO: 346), TVAAPSVFIFPP (SEQ ID NO: 347), QPKAAP (SEQ ID NO: 348), QPKAAPSVTLFPP (SEQ ID NO: 349), AKTTPP (SEQ ID NO: 350), AKTTPPSVTPLAP (SEQ ID NO: 351), AKTTAP (SEQ ID NO: 352), AKTTAPSVYPLAP (SEQ ID NO: 353), ASTKGP (SEQ ID NO: 354), ASTKGPSVFPLAP (SEQ ID NO: 355), GENKVEYAPALMALS (SEQ ID NO: 356), GPAKELTPLKEAKVS (SEQ ID NO: 357), and GHEAAAVMQVQYPAS (SEQ ID NO: 358) or any combination thereof (see WO2007/024715).

### Dimerization Specific Amino Acids

In one embodiment, the multivalent antibody comprises at least one dimerization specific amino acid change. The dimerization specific amino acid changes result in “knobs into holes” interactions, and increases the assembly of correct multivalent antibodies. The dimerization specific amino acids can be within the CH1 domain or the CL domain or combinations thereof. The dimerization specific amino acids used to pair CH1 domains with other CH1 domains (CH1-CH1) and CL domains with other CL domains (CL-CL) and can be found at least in the disclosures of WO2014082179, WO2015181805 family and WO2017059551. The dimerization specific amino acids can also be within the Fc domain and can be in combination with dimerization specific amino acids within the CH1 or CL domains. In one embodiment, the disclosure provides a bispecific antibody comprising at least one dimerization specific amino acid pair.

### Further Alteration of the Framework of Fc Region

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al.

In yet another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcγRI, FcγRII, FcγRIII and FcRn have been mapped and variants with improved binding have been described (see Shields et al., J. Biol. Chem. 276:6591-6604, 2001).

In still another aspect, the glycosylation of the multispecific antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al., describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). WO99/54342 by Umana et al., describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another aspect, if a reduction of ADCC is desired, human antibody subclass IgG4 was shown in many previous reports to have only modest ADCC and almost no CDC effector function (Moore G L, et al., 2010 MAbs, 2:181-189). However, natural IgG4 was found less stable in stress conditions such as in acidic buffer or under increasing temperature (Angal, S. 1993 Mol Immunol, 30:105-108; Dall'Acqua, W. et al., 1998 Biochemistry, 37:9266-9273; Aalberse et al., 2002 Immunol, 105:9-19). Reduced ADCC can be achieved by operably linking the antibody to an IgG4 Fc engineered with combinations of alterations that reduce FcγR binding or C1q binding activities, thereby reducing or eliminating ADCC and CDC effector functions. Considering the physicochemical properties of antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., 2007 Science, 317:1554-157). The mutation of serine to proline at position 228 (EU numbering system) appeared inhibitory to the IgG4 heavy chain separation (Angal, S. 1993 Mol Immunol, 30:105-108; Aalberse et al., 2002 Immunol, 105:9-19). Some of the amino acid residues in the hinge and γFc region were reported to have impact on antibody interaction with Fcγ receptors (Chappel S M, et al., 1991 Proc. Natl. Acad. Sci. USA, 88:9036-9040; Mukherjee, J. et al., 1995 FASEB J, 9:115-119; Armour, K. L. et al., 1999 Eur J Immunol, 29:2613-2624; Clynes, R. A. et al., 2000 Nature Medicine, 6:443-446; Arnold J. N., 2007 Annu Rev immunol, 25:21-50). Furthermore, some rarely occurring IgG4 isoforms in human population can also elicit different physicochemical properties (Brusco, A. et al., 1998 Eur J Immunogenet, 25:349-55; Aalberse et al., 2002 Immunol, 105:9-19). To generate multispecific antibodies with low ADCC and CDC but with good stability, it is possible to modify the hinge and Fc region of human IgG4 and introduce a number of alterations. These modified IgG4 Fc molecules can be found in SEQ ID NOs: 83-88, U.S. Pat. No. 8,735,553 to Li et al.

### Antibody Production

Antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NO: 16, SEQ ID NO: 33, SEQ ID NO: 50, SEQ ID NO: 61, SEQ ID NO: 71, SEQ ID NO: 76, SEQ ID NO: 85 and SEQ ID NO: 87. In some aspects, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NOs:17, 34, or 51.

The polynucleotides of the present disclosure can encode the variable region sequence of an anti-CEA×CD137 antibody. They can also encode both a variable region and a constant region of the antibody. Some of the polynucleotide sequences encode a polypeptide that comprises variable regions of both the heavy chain and the light chain of the exemplified anti-CEA×CD137 antibodies.

Also provided in the present disclosure are expression vectors and host cells for producing the anti-CEA×CD137 antibodies. The choice of expression vector depends on the intended host cells in which the vector is to be expressed. Typically, the expression vectors contain a promoter and other regulatory sequences (e.g., enhancers) that are operably linked to the polynucleotides encoding an anti-CEA×CD137 antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an anti-CEA×CD137 antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-CEA×CD137 antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-CEA×CD137 antibodies. Insect cells in combination with baculovirus vectors can also be used. In other aspects, mammalian host cells are used to express and produce the anti-CEA×CD137 antibodies of the present disclosure. For example, they can be either a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### Production of Bispecific Antibodies

The current standard for an engineered heterodimeric antibody Fc domain is the knobs-into-holes (KiH) design, which introduced mutations at the core CH3 domain interface. The resulted heterodimers have a reduced CH3 melting temperature (69° C. or less). On the contrary, the ZW heterodimeric Fc design has a thermal stability of 81.5° C., which is comparable to the wild-type CH3 domain.

### Methods of Detection and Diagnosis

The antibodies or antigen-binding fragments of the present disclosure are useful in a variety of applications including, but not limited to, methods for the detection of CEA. In one aspect, the antibodies or antigen-binding fragments are useful for detecting the presence of CEA in a biological sample. The term “detecting” as used herein includes quantitative or qualitative detection. In certain aspects, a biological sample comprises a cell or tissue. In other aspects, such tissues include normal and/or cancerous tissues that express CEA at higher levels relative to other tissues.

In one aspect, the present disclosure provides a method of detecting the presence of CEA in a biological sample. In certain aspects, the method comprises contacting the biological sample with an anti-CEA×CD137 antibody under conditions permissive for binding of the antibody to the antigen and detecting whether a complex is formed between the antibody and the antigen. The biological sample can include, without limitation, urine, tissue, sputum or blood samples.

Also included is a method of diagnosing a disorder associated with expression of CEA. In certain aspects, the method comprises contacting a test cell with an anti-CEA×CD137 antibody; determining the level of expression (either quantitatively or qualitatively) of CEA expressed by the test cell by detecting binding of the anti-CEA×CD137 antibody to the CEA polypeptide; and comparing the level of expression by the test cell with the level of CEA expression in a control cell (e.g., a normal cell of the same tissue origin as the test cell or a non-CEA expressing cell), wherein a higher level of CEA expression in the test cell as compared to the control cell indicates the presence of a disorder associated with expression of CEA.

### Methods of Treatment

The antibodies or antigen-binding fragments of the present disclosure are useful in a variety of applications including, but not limited to, methods for the treatment of a CEA-associated disorder or disease. In one aspect, the CEA-associated disorder or disease is a cancer.

In one aspect, the present disclosure provides a method of treating cancer. In certain aspects, the method comprises administering to a patient in need an effective amount of an anti-CEA×CD137 antibody or antigen-binding fragment. The cancer can include, without limitation, gastric cancer, colon cancer, pancreatic cancer, breast cancer, head and neck cancer, kidney cancer, liver cancer, small cell lung cancer, non-small cell lung cancer, ovarian cancer, skin cancer, mesothelioma, lymphoma, leukemia, myeloma and sarcoma.

The antibody or antigen-binding fragment as disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or antigen-binding fragments of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or antigen-binding fragment of the disclosure will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 100 mg/kg of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses can be administered intermittently, e.g. every week or every three weeks (e.g. such that the patient receives from about two to about twenty, or e.g. about six doses of the antibody). An initial higher loading dose, followed by one or more lower doses can be administered. However, other dosage regimens can be useful. The progress of this therapy is easily monitored by conventional techniques and assays.

### Combination Therapy

In one aspect, anti-CEA×CD137 antibodies of the present disclosure can be used in combination with other therapeutic agents. Other therapeutic agents that can be used with the anti-CEA×CD137 antibodies of the present disclosure include: but are not limited to, a chemotherapeutic agent (e.g., paclitaxel or a paclitaxel agent; (e.g. Abraxane®), docetaxel; carboplatin; topotecan; cisplatin; irinotecan, doxorubicin, lenalidomide, 5-azacytidine, ifosfamide, oxaliplatin, pemetrexed disodium, cyclophosphamide, etoposide, decitabine, fludarabine, vincristine, bendamustine, chlorambucil, busulfan, gemcitabine, melphalan, pentostatin, mitoxantrone, pemetrexed disodium), tyrosine kinase inhibitor (e.g., EGFR inhibitor (e.g., erlotinib), multikinase inhibitor (e.g., MGCD265, RGB-286638), CD-20 targeting agent (e.g., rituximab, ofatumumab, R05072759, LFB-R603), CD52 targeting agent (e.g., alemtuzumab), prednisolone, darbepoetin alfa, lenalidomide, Bcl-2 inhibitor (e.g., oblimersen sodium), aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), EGEN-001, Polo-like kinase 1 inhibitor (e.g., BI 672).

Anti-CEA×CD137 antibodies of the present disclosure can be used in combination with other therapeutics, for example, immune checkpoint antibodies. Such immune checkpoint antibodies can include anti-PD1 antibodies. Anti-PD1 antibodies can include, without limitation, Tislelizumab, Pembrolizumab or Nivolumab. Tislelizumab is disclosed in U.S. Pat. No. 8,735,553. Pembrolizumab (formerly MK-3475), is disclosed in U.S. Pat. Nos. 8,354,509 and 8,900,587 and is a humanized lgG4-K immunoglobulin which targets the PD1 receptor and inhibits binding of the PD1 receptor ligands PD-L1 and PD-L2. Pembrolizumab has been approved for the indications of metastatic melanoma and metastatic non-small cell lung cancer (NSCLC) and is under clinical investigation for the treatment of head and neck squamous cell carcinoma (HNSCC), and refractory Hodgkin's lymphoma (cHL). Nivolumab (as disclosed by Bristol-Meyers Squibb) is a fully human lgG4-K monoclonal antibody. Nivolumab (clone 5C4) is disclosed in U.S. Pat. No. 8,008,449 and WO 2006/121168. Nivolumab is approved for the treatment of melanoma, lung cancer, kidney cancer, and Hodgkin's lymphoma.

Other immune checkpoint antibodies for combination with anti-CEA×CD137 antibodies can include anti-TIGIT antibodies. Such anti-TIGIT antibodies can include without limitation, anti-TIGIT antibodies as disclosed in WO2019/129261.

### Pharmaceutical Compositions and Formulations

Also provided are compositions, including pharmaceutical formulations, comprising an anti-CEA×CD137 antibody or antigen-binding fragment thereof, or polynucleotides comprising sequences encoding an anti-CEA×CD137 antibody or antigen-binding fragment. In certain embodiments, compositions comprise one or more anti-CEA×CD137 antibodies or antigen-binding fragments, or one or more polynucleotides comprising sequences encoding one or more anti-CEA×CD137 antibodies or antigen-binding fragments. These compositions can further comprise suitable carriers, such as pharmaceutically acceptable excipients including buffers, which are well known in the art.

Pharmaceutical formulations of an anti-CEA×CD137 antibody or antigen-binding fragment as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

In one embodiment, the formulation is composed of L-histidine/L-histidine hydrochloride monohydrate, trehalose and polysorbate 20. In another embodiment the concentration of the anti-CEA×CD137 antibody drug product, after constitution with sterile water for injection, is an isotonic solution consisting of 10 mg/mL anti-CEA×CD137 antibody, 20 mM histidine/histidine HCl, 240 mM trehalose dihydrate, and 0.02% polysorbate 20, at a pH of approximately 5.5.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility can be readily accomplished, e.g., by filtration through sterile filtration membranes.

### EXAMPLES

### Example 1: Generation of Anti-CEA Monoclonal Antibody

### CEA Recombinant Proteins for Immunization and Binding Assays

To discover new antibodies against CEA that both cross-react with human and Macaca mulatta CEA in the membrane peripheral region containing domain B3, (amino acids 596-674 of SEQ ID NO:88, see Beauchemin et al., Mol. Cell Bio., 1987, 7(9):3321-3330)). but without off-target binding with other human CEACAM members, several recombinant proteins were designed and expressed for antibody screening (see Table 3).

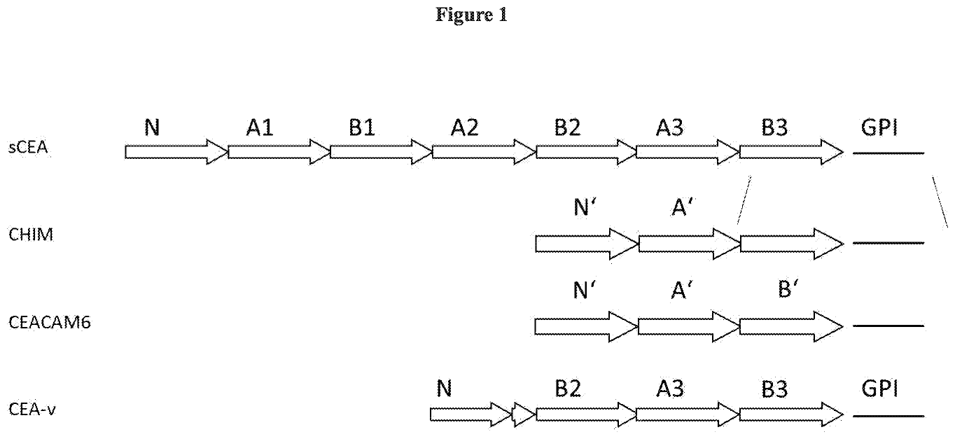

The cDNA coding regions for the full-length human CEA (SEQ ID NO: 88), Macaca CEA (SEQ ID NO: 89) and the full-length human CEACAM6 (SEQ ID NO: 90) were ordered based on the GenBank sequence. For human CEA (Accession No: NM_004363.2), the gene is available from Sinobio, Cat. No. HG11077-UT. For Macaca CEA (Accession No: NM_001047125), the gene is available from Genscript, Cat. No. OMb23865D. For human CEACAM6 (Accession No: NM_002483.4), the gene is available from Sinobio, Cat. No. HG10823-UT. The schematic presentation of CEA fusion proteins is shown in FIG. 1. It is reported a splice variant of human CEA is expressed concomitantly with full-length CEA on tumors (Peng et al., PloS one, 7, e36412-e36412 (2012)), and the variant (CEA-v) was prepared accordingly. To generate this construct, the coding region of extracellular domain (ECD) consisting of amino acid (AA) 1-687 of huCEA (SEQ ID NO: 91), the region of amino acid (AA) 1-690 of monkeyCEA (SEQ ID NO: 92) and the region of amino acid (AA) 1-320 of CEACAM6 (SEQ ID NO:93) were PCR-amplified. The regions of CEA amino acid (AA) 1-78 (SEQ ID NO:94) and amino acids 398-687 of CEA (SEQ ID NO:95) were PCR-amplified, and then conjugated by overlap-PCR to make a CEA variant (CEA-v) (SEQ ID NO: 96). Alternatively, the regions of CEACAM6 amino acid (AA) 1-273 (SEQ ID NO: 97), and the membrane-peripheral region containing domain B3 of CEA amino acid (AA) 596-687 of (SEQ ID NO: 98) were PCR-amplified, and then conjugated by overlap-PCR to make a chimeric construct (CHIM) (SEQ ID NO: 99). All constructs were then cloned into a pcDNA3.1-based expression vector (Invitrogen, Carlsbad, CA, USA) with C-terminus fused with 6×His tags individually, which resulted in five recombinant fusion protein expression plasmids, CEA, monkeyCEA, CEACAM6, CEA-v and CHIM. For recombinant fusion protein production, CEA, monkeyCEA, CEACAM6, CEA-v and CHIM plasmids were transiently transfected into a HEK293-based mammalian cell expression system (generated in house) and cultured for 5-7 days in a CO₂ incubator equipped with a rotating shaker. The supernatants containing the recombinant proteins were collected and cleared by centrifugation. Recombinant proteins were purified using a Ni-NTA agarose (Cat. No. R90115, Invitrogen). All recombinant proteins were dialyzed against phosphate buffered saline (PBS) and stored in −80° C. freezer in small aliquots.

### Stable Expression in Cell Lines

To establish stable cell lines that express full-length human CEA (Accession No: NM_004363.2, the cDNA expressing CEA was cloned into a retroviral vector pFB-Neo (Cat. No. 217561, Agilent, USA). Dual-tropic retroviral vectors were generated according to a previous protocol (Zhang et al., Blood. 2005 106(5):1544-51). Viral vectors containing human CEA were transduced into L929 (ATCC, Manassas, VA, USA) and CT26 cells (ATCC, Manassas, VA, USA), in order to generate human CEA expressing cell lines. The high expression cell lines were selected by culture in complete RPMI1640 medium containing 10% FBS with G418, and then verified via FACS binding assay.

### Immunization, Hybridoma Fusion and Cloning

Eight to twelve week-old Balb/c mice (HFK BIOSCIENCE CO., LTD, Beijing, China) were immunized intraperitoneally (i.p.) with 500 μl of 1×10⁷ L929/huCEA cells with or without a water-soluble adjuvant (Cat. No. KX0210041, KangBiQuan, Beijing, China). The procedure was repeated two weeks later in order to boost antibody production. Two weeks after the third immunization, mouse sera were evaluated for soluble CEA (sCEA) binding by ELISA and FACS. Splenocytes were isolated and fused to the murine myeloma cell line, SP2/0 cells (ATCC, Manassas, VA, USA), using the standard techniques (Colligan J E, et al., CURRENT PROTOCOLS IN IMMUNOLOGY, 1993).

### Assessment of CEA Binding Activity of Antibodies by ELISA and FACS

To screen for antibodies that bound human CEA, but did not bind CEACAM6 or sCEA, antibodies which bound to CHIM but not to sCEA, CEACAM6 and CEA-v, and antibodies which bind to CHIM, sCEA and CEA-v, but not to CEACAM6 were screened and counter-screened. The supernatants of hybridoma clones were initially screened by ELISA as described in (Methods in Molecular Biology (2007) 378:33-52) with some modifications. Briefly, sCEA, CHIM, CEACAM6 or CEA-v were coated in 96-well plates at a low concentration of 3 μg/ml, individually. The HRP-linked anti-mouse IgG antibody (Cat. No. 7076S, Cell Signaling Technology, USA) and substrate (Cat. No. 00-4201-56, eBioscience, USA) were used for development, and absorbance signal at the wavelength of 450 nm was measured using a plate reader (SpectraMax Paradigm™, Molecular Devices, USA). The ELISA-positive clones were further verified by FACS using either the L929/huCEA and/or MKN45 cells (ATCC). MKN45 cells are of human gastric cancer origin. CEA-expressing cells (10′ cells/well) were incubated with ELISA-positive hybridoma supernatants, followed by binding with Alexa Fluro-647-labeled goat anti-mouse IgG antibody (Cat. No. A0473, Beyotime Biotechnology, China). Cell fluorescence was quantified using a flow cytometer (Guava easyCyte™ 8HT, Merck-Millipore, USA).

The conditioned media from the hybridomas that showed positive signals in FACS screening, and binding to CHIM but not CEACAM6 and sCEA were subjected to functional assays to evaluate the presence of sCEA on the binding of CEA antibodies to CEA expressing cells (see the Examples below). The antibodies with the desired binding specificity and functional activities were further sub-cloned and characterized.

### Subcloning and Adaptation of Hybridomas to Serum-Free or Low Serum Medium

After screening primarily by ELISA, FACS and functional assays, the positive hybridoma clones were sub-cloned by limiting dilution. The top antibody subclones verified through functional assays were adapted for growth in the CDM4MAb medium (Cat. No. SH30801.02, Hyclone, USA) with 3% FBS.

### Expression and Purification of Monoclonal Antibodies

Hybridoma cells were cultured in CDM4MAb medium (Cat. No. SH30801.02, Hyclone), and incubated in a CO₂ incubator for 5 to 7 days at 37° C. The conditioned medium was collected through centrifugation and filtration by passing through a 0.22 μm membrane before purification. Murine antibody-containing supernatants were applied and bound to a Protein A column (Cat. No. 17127901, GE Life Sciences) following the protocol in the manufacturer's guide. The procedure usually yielded antibodies at purity above 90%. The Protein A-affinity purified antibodies were either dialyzed against PBS or further purified using a HiLoad™ 16/60 Superdex™200 column (Cat. No. 17531801, GE Life Sciences) to remove aggregates. Protein concentrations were determined by measuring absorbance at 280 nm. The final antibody preparations were stored in aliquots in −80° C. freezer.

TABLE 3
amino acid and nucleic acid sequences

| SEQ ID | | |
|---|---|---|
| NO: | Construct | SEQUENCE |
| SEQ ID | full-length | MESPSAPPHRWCIPWQRLLLTASLLTFWNPPTTAKLTIESTPFNV |
| NO: 88 | human | AEGKEVLLLVHNLPQHLFGYSWYKGERVDGNRQIIGYVIGTQQ |
| | CEA | ATPGPAYSGREIIYPNASLLIQNIIQNDTGFYTLHVIKSDLVNEEA |
| | | TGQFRVYPELPKPSISSNNSKPVEDKDAVAFTCEPETQDATYLW |
| | | WVNNQSLPVSPRLQLSNGNRTLTLFNVTRNDTASYKCETQNPV |
| | | SARRSDSVILNVLYGPDAPTISPLNTSYRSGENLNLSCHAASNPP |
| | | AQYSWFVNGTFQQSTQELFIPNITVNNSGSYTCQAHNSDTGLN |
| | | RTTVTTITVYAEPPKPFITSNNSNPVEDEDAVALTCEPEIQNTTYL |
| | | WWVNNQSLPVSPRLQLSNDNRTLTLLSVTRNDVGPYECGIQNE |
| | | LSVDHSDPVILNVLYGPDDPTISPSYTYYRPGVNLSLSCHAASN |
| | | PPAQYSWLIDGNIQQHTQELFISNITEKNSGLYTCQANNSASGH |
| | | SRTTVKTITVSAELPKPSISSNNSKPVEDKDAVAFTCEPEAQNTT |
| | | YLWWVNGQSLPVSPRLQLSNGNRTLTLFNVTRNDARAYVCGI |
| | | QNSVSANRSDPVTLDVLYGPDTPIISPPDSSYLSGANLNLSCHSA |
| | | SNPSPQYSWRINGIPQQHTQVLFIAKITPNNNGTYACFVSNLAT |
| | | GRNNSIVKSITVSASGTSPGLSAGATVGIMIGVLVGVALI |
| SEQ ID | Macaca | MGSPSAPLHRWCIPWQTLLLTASLLTFWNPPTTAQLTIESRPFNV |
| NO: 89 | CEA | AEGKEVLLLAHNVSQNLFGYIWYKGERVDASRRIGSCVIRTQQI |
| | | TPGPAHSGRETIDFNASLLIHNVTQSDTGSYTIQVIKEDLVNEEA |
| | | TGQFRVYPELPKPYISSNNSNPVEDKDAVALTCEPETQDTTYLW |
| | | WVNNQSLPVSPRLELSSDNRTLTVFNIPRNDTTSYKCETQNPVS |
| | | VRRSDPVTLNVLYGPDAPTISPLNTPYRAGENLNLTCHAASNPT |
| | | AQYFWFVNGTFQQSTQELFIPNITVNNSGSYMCQAHNSATGLN |
| | | RTTVTAITVYAELPKPYITSNNSNPIEDKDAVTLTCEPETQDTTY |
| | | LWWVNNQSLSVSSRLELSNDNRTLTVFNIPRNDTTFYECETQNP |
| | | VSVRRSDPVTLNVLYGPDAPTISPLNTPYRAGENLNLSCHAASN |
| | | PAAQYSWFVNGTFQQSTQELFIPNITVNNSGSYMCQAHNSATG |
| | | LNRTTVTAITVYVELPKPYISSNNSNPIEDKDAVTLTCEPVAENT |
| | | TYLWWVNNQSLSVSPRLQLSNGNRILTLLSVTRNDTGPYECGI |
| | | QNSESAKRSDPVTLNVTYGPDTPIISPPDLSYRSGANLNLSCHS |
| | | DSNPSPQYSWLINGTLRQHTQVLFISKITSNNSGAYACFVSNLAT |
| | | GRNNSIVKNISVSSGDSAPGSSGLSARATVGIIIGMLVGVALM |
| SEQ ID | full-length | MGPPSAPPCRLHVPWKEVLLTASLLTFWNPPTTAKLTIESTPFNV |
| NO: 90 | human | AEGKEVLLLAHNLPQNRIGYSWYKGERVDGNSLIVGYVIGTQQ |
| | CEACAM | ATPGPAYSGRETIYPNASLLIQNVTQNDTGFYTLQVIKSDLVNEE |
| | 6 | ATGQFHVYPELPKPSISSNNSNPVEDKDAVAFTCEPEVQNTTYL |
| | | WWVNGQSLPVSPRLQLSNGNMTLTLLSVKRNDAGSYECEIQN |
| | | PASANRSDPVTLNVLYGPDVPTISPSKANYRPGENLNLSCHAAS |
| | | NPPAQYSWFINGTFQQSTQELFIPNITVNNSGSYMCQAHNSATG |
| | | LNRTTVTMITVSGSAPVLSAVATVGITIGVLARVALI |
| SEQ ID | 1-687 of | MESPSAPPHRWCIPWQRLLLTASLLTFWNPPTTAKLTIESTPFNV |
| NO: 91 | huCEA | AEGKEVLLLVHNLPQHLFGYSWYKGERVDGNRQIIGYVIGTQQ |
| | | ATPGPAYSGREIIYPNASLLIQNIIQNDTGFYTLHVIKSDLVNEEA |
| | | TGQFRVYPELPKPSISSNNSKPVEDKDAVAFTCEPETQDATYLW |
| | | WVNNQSLPVSPRLQLSNGNRTLTLFNVTRNDTASYKCETQNPV |
| | | SARRSDSVILNVLYGPDAPTISPLNTSYRSGENLNLSCHAASNPP |
| | | AQYSWFVNGTFQQSTQELFIPNITVNNSGSYTCQAHNSDTGLN |
| | | RTTVTTITVYAEPPKPFITSNNSNPVEDEDAVALTCEPEIQNTTYL |
| | | WWVNNQSLPVSPRLQLSNDNRTLTLLSVTRNDVGPYECGIQNE |
| | | LSVDHSDPVILNVLYGPDDPTISPSYTYYRPGVNLSLSCHAASN |
| | | PPAQYSWLIDGNIQQHTQELFISNITEKNSGLYTCQANNSASGH |
| | | SRTTVKTITVSAELPKPSISSNNSKPVEDKDAVAFTCEPEAQNTT |
| | | YLWWVNGQSLPVSPRLQLSNGNRTLTLFNVTRNDARAYVCGI |
| | | QNSVSANRSDPVTLDVLYGPDTPIISPPDSSYLSGANLNLSCHSA |
| | | SNPSPQYSWRINGIPQQHTQVLFIAKITPNNNGTYACFVSNLAT |
| | | GRNNSIVKSITVSASGTSPGLSAGAHHHHHH |
| SEQ ID | 1-690 of | MGSPSAPLHRWCIPWQTLLLTASLLTFWNPPTTAQLTIESRPFNV |
| NO: 92 | monkey | AEGKEVLLLAHNVSQNLFGYIWYKGERVDASRRIGSCVIRTQQI |
| | CEA | TPGPAHSGRETIDFNASLLIHNVTQSDTGSYTIQVIKEDLVNEEA |
| | | TGQFRVYPELPKPYISSNNSNPVEDKDAVALTCEPETQDTTYLW |
| | | WVNNQSLPVSPRLELSSDNRTLTVFNIPRNDTTSYKCETQNPVS |
| | | VRRSDPVTLNVLYGPDAPTISPLNTPYRAGENLNLTCHAASNPT |
| | | AQYFWFVNGTFQQSTQELFIPNITVNNSGSYMCQAHNSATGLN |
| | | RTTVTAITVYAELPKPYITSNNSNPIEDKDAVTLTCEPETQDTTY |
| | | LWWVNNQSLSVSSRLELSNDNRTLTVFNIPRNDTTFYECETQNP |
| | | VSVRRSDPVTLNVLYGPDAPTISPLNTPYRAGENLNLSCHAASN |
| | | PAAQYSWFVNGTFQQSTQELFIPNITVNNSGSYMCQAHNSATG |
| | | LNRTTVTAITVYVELPKPYISSNNSNPIEDKDAVTLTCEPVAENT |
| | | TYLWWVNNQSLSVSPRLQLSNGNRILTLLSVTRNDTGPYECGI |
| | | QNSESAKRSDPVTLNVTYGPDTPIISPPDLSYRSGANLNLSCHS |
| | | DSNPSPQYSWLINGTLRQHTQVLFISKITSNNSGAYACFVSNLAT |
| | | GRNNSIVKNISVSSGDSAPGSSGLSARAHHHHHH |
| SEQ ID | 1-320 of | MGPPSAPPCRLHVPWKEVLLTASLLTFWNPPTTAKLTIESTPFNV |
| NO: 93 | CEACAM | AEGKEVLLLAHNLPQNRIGYSWYKGERVDGNSLIVGYVIGTQQ |
| | 6 | ATPGPAYSGRETIYPNASLLIQNVTQNDTGFYTLQVIKSDLVNEE |
| | | ATGQFHVYPELPKPSISSNNSNPVEDKDAVAFTCEPEVQNTTYL |
| | | WWVNGQSLPVSPRLQLSNGNMTLTLLSVKRNDAGSYECEIQN |
| | | PASANRSDPVTLNVLYGPDVPTISPSKANYRPGENLNLSCHAAS |
| | | NPPAQYSWFINGTFQQSTQELFIPNITVNNSGSYMCQAHNSATG |
| | | LNRTTVTMITVSGHHHHHH |
| SEQ ID | 1-78 of | MESPSAPPHRWCIPWQRLLLTASLLTFWNPPTTAKLTIESTPFNV |
| NO: 94 | huCEA | AEGKEVLLLVHNLPQHLFGYSWYKGERVDGNRQIIGYVIGTQQ |
| | | ATPGPAYSGREIIYPNASLLIQNIIQN |
| SEQ ID | 398-687 of | ELSVDHSDPVILNVLYGPDDPTISPSYTYYRPGVNLSLSCHAAS |
| NO: 95 | huCEA | NPPAQYSWLIDGNIQQHTQELFISNITEKNSGLYTCQANNSASG |
| | | HSRTTVKTITVSAELPKPSISSNNSKPVEDKDAVAFTCEPEAQNT |
| | | TYLWWVNGQSLPVSPRLQLSNGNRTLTLFNVTRNDARAYVCGI |
| | | QNSVSANRSDPVTLDVLYGPDTPIISPPDSSYLSGANLNLSCHSA |
| | | SNPSPQYSWRINGIPQQHTQVLFIAKITPNNNGTYACFVSNLAT |
| | | GRNNSIVKSITVSASGTSPGLSAGA |
| SEQ ID | CEA-v | MESPSAPPHRWCIPWQRLLLTASLLTFWNPPTTAKLTIESTPFNV |
| NO: 96 | | AEGKEVLLLVHNLPQHLFGYSWYKGERVDGNRQIIGYVIGTQQ |
| | | ATPGPAYSGREIIYPNASLLIQNIIQNELSVDHSDPVILNVLYGPD |
| | | DPTISPSYTYYRPGVNLSLSCHAASNPPAQYSWLIDGNIQQHTQ |
| | | ELFISNITEKNSGLYTCQANNSASGHSRTTVKTITVSAELPKPSIS |
| | | SNNSKPVEDKDAVAFTCEPEAQNTTYLWWVNGQSLPVSPRLQL |
| | | SNGNRTLTLFNVTRNDARAYVCGIQNSVSANRSDPVTLDVLYG |
| | | PDTPIISPPDSSYLSGANLNLSCHSASNPSPQYSWRINGIPQQHT |
| | | QVLFIAKITPNNNGTYACFVSNLATGRNNSIVKSITVSASGTSPG |
| | | LSAGAHHHHHH |
| SEQ ID | 1-273 of | MGPPSAPPCRLHVPWKEVLLTASLLTFWNPPTTAKLTIESTPFNV |
| NO: 97 | CEACAM | AEGKEVLLLAHNLPQNRIGYSWYKGERVDGNSLIVGYVIGTQQ |
| | 6 | ATPGPAYSGRETIYPNASLLIQNVTQNDTGFYTLQVIKSDLVNEE |
| | | ATGQFHVYPELPKPSISSNNSNPVEDKDAVAFTCEPEVQNTTYL |
| | | WWVNGQSLPVSPRLQLSNGNMTLTLLSVKRNDAGSYECEIQN |
| | | PASANRSDPVTLNVLYGPDVPTIS |
| SEQ ID | 596-687 of | PIISPPDSSYLSGANLNLSCHSASNPSPQYSWRINGIPQQHTQVL |
| NO: 98 | huCEA | FIAKITPNNNGTYACFVSNLATGRNNSIVKSITVSASGTSPGLSA |
| | | GA |
| SEQ ID | CHIM | MGPPSAPPCRLHVPWKEVLLTASLLTFWNPPTTAKLTIESTPFNV |
| NO: 99 | | AEGKEVLLLAHNLPQNRIGYSWYKGERVDGNSLIVGYVIGTQQ |
| | | ATPGPAYSGRETIYPNASLLIQNVTQNDTGFYTLQVIKSDLVNEE |
| | | ATGQFHVYPELPKPSISSNNSNPVEDKDAVAFTCEPEVQNTTYL |
| | | WWVNGQSLPVSPRLQLSNGNMTLTLLSVKRNDAGSYECEIQN |
| | | PASANRSDPVTLNVLYGPDVPTISPIISPPDSSYLSGANLNLSCHS |
| | | ASNPSPQYSWRINGIPQQHTQVLFIAKITPNNNGTYACFVSNLA |
| | | TGRNNSIVKSITVSASGTSPGLSAGAHHHHHH |
| SEQ ID | CEACAM | MGHLSAPLHRVRVPWQGLLLTASLLTFWNPPTTAQLTTESMPF |
| NO: 100 | 1 | NVAEGKEVLLLVHNLPQQLFGYSWYKGERVDGNRQIVGYAIG |
| | | TQQATPGPANSGRETIYPNASLLIQNVTQNDTGFYTLQVIKSDL |
| | | VNEEATGQFHVYPELPKPSISSNNSNPVEDKDAVAFTCEPETQD |
| | | TTYLWWINNQSLPVSPRLQLSNGNRTLTLLSVTRNDTGPYECEI |
| | | QNPVSANRSDPVTLNVTYGPDTPTISPSDTYYRPGANLSLSCYA |
| | | ASNPPAQYSWLINGTFQQSTQELFIPNITVNNSGSYTCHANNSV |
| | | TGCNRTTVKTIIVTELSPVVAKPQIKASKTTVTGDKDSVNLTCST |
| | | NDTGISIRWFFKNQSLPSSERMKLSQGNTTLSINPVKREDAGTY |
| | | WCEVFNPISKNQSDPIMLNVNYNALPQENGLSPGAIAGIVIGVV |
| | | ALVALIAVALACFLHFGKTGSSGPLQ |
| SEQ ID | CEACAM | MGPPSASPHRECIPWQGLLLTASLLNFWNPPTTAKLTIESMPLSV |
| NO: 101 | 3 | AEGKEVLLLVHNLPQHLFGYSWYKGERVDGNSLIVGYVIGTQ |
| | | QATPGAAYSGRETIYTNASLLIQNVTQNDIGFYTLQVIKSDLVN |
| | | EEATGQFHVYQENAPGLPVGAVAGIVTGVLVGVALVAALVCFL |
| | | LLAKTGRTSIQRDLKEQQPQALAPGRGPSHSSAFSMSPLSTAQA |
| | | PLPNPRTAASIYEELLKHDTNIYCRMDHKAEVAS |
| SEQ ID | CEACAM | MGSPSACPYRVCIPWQGLLLTASLLTFWNLPNSAQTNIDVVPFN |
| NO: 102 | 7 | VAEGKEVLLVVHNESQNLYGYNWYKGERVHANYRIIGYVKNI |
| | | SQENAPGPAHNGRETIYPNGTLLIQNVTHNDAGIYTLHVIKENL |
| | | VNEEVTRQFYVFSEPPKPSITSNNFNPVENKDIVVLTCQPETQNT |
| | | TYLWWVNNQSLLVSPRLLLSTDNRTLVLLSATKNDIGPYECEIQ |
| | | NPVGASRSDPVTLNVRYESVQASSPDLSAGTAVSIMIGVLAGM |
| | | ALI |
| SEQ ID | CEACAM | MGPISAPSCRWRIPWQGLLLTASLFTFWNPPTTAQLTIEAVPSNA |
| NO: 103 | 8 | AEGKEVLLLVHNLPQDPRGYNWYKGETVDANRRIIGYVISNQQ |
| | | ITPGPAYSNRETIYPNASLLMRNVTRNDTGSYTLQVIKLNLMSE |
| | | EVTGQFSVHPETPKPSISSNNSNPVEDKDAVAFTCEPETQNTTYL |
| | | WWVNGQSLPVSPRLQLSNGNRTLTLLSVTRNDVGPYECEIQNP |
| | | ASANFSDPVTLNVLYGPDAPTISPSDTYYHAGVNLNLSCHAAS |
| | | NPPSQYSWSVNGTFQQYTQKLFIPNITTKNSGSYACHTTNSATG |
| | | RNRTTVRMITVSDALVQGSSPGLSARATVSIMIGVLARVALI |
| SEQ ID | full-length | ATGGAGTCTCCCTCGGCCCCTCCCCACAGATGGTGCATCCCC |
| NO: 104 | human | TGGCAGAGGCTCCTGCTCACAGCCTCACTTCTAACCTTCTGG |
| CEA DNA | | AACCCGCCCACCACTGCCAAGCTCACTATTGAATCCACGCCG |
| | | TTCAATGTCGCAGAGGGGAAGGAGGTGCTTCTACTTGTCCAC |
| | | AATCTGCCCCAGCATCTTTTTGGCTACAGCTGGTACAAAGGT |
| | | GAAAGAGTGGATGGCAACCGTCAAATTATAGGATATGTAATA |
| | | GGAACTCAACAAGCTACCCCAGGGCCCGCATACAGTGGTCG |
| | | AGAGATAATATACCCCAATGCATCCCTGCTGATCCAGAACATC |
| | | ATCCAGAATGACACAGGATTCTACACCCTACACGTCATAAAG |
| | | TCAGATCTTGTGAATGAAGAAGCAACTGGCCAGTTCCGGGT |
| | | ATACCCGGAGCTGCCCAAGCCCTCCATCTCCAGCAACAACTC |
| | | CAAACCCGTGGAGGACAAGGATGCTGTGGCCTTCACCTGTG |
| | | AACCTGAGACTCAGGACGCAACCTACCTGTGGTGGGTAAAC |
| | | AATCAGAGCCTCCCGGTCAGTCCCAGGCTGCAGCTGTCCAAT |
| | | GGCAACAGGACCCTCACTCTATTCAATGTCACAAGAAATGAC |
| | | ACAGCAAGCTACAAATGTGAAACCCAGAACCCAGTGAGTGC |
| | | CAGGCGCAGTGATTCAGTCATCCTGAATGTCCTCTATGGCCC |
| | | GGATGCCCCCACCATTTCCCCTCTAAACACATCTTACAGATCA |
| | | GGGGAAAATCTGAACCTCTCCTGCCACGCAGCCTCTAACCC |
| | | ACCTGCACAGTACTCTTGGTTTGTCAATGGGACTTTCCAGCA |
| | | ATCCACCCAAGAGCTCTTTATCCCCAACATCACTGTGAATAAT |
| | | AGTGGATCCTATACGTGCCAAGCCCATAACTCAGACACTGGC |
| | | CTCAATAGGACCACAGTCACGACGATCACAGTCTATGCAGAG |
| | | CCACCCAAACCCTTCATCACCAGCAACAACTCCAACCCCGT |
| | | GGAGGATGAGGATGCTGTAGCCTTAACCTGTGAACCTGAGAT |
| | | TCAGAACACAACCTACCTGTGGTGGGTAAATAATCAGAGCCT |
| | | CCCGGTCAGTCCCAGGCTGCAGCTGTCCAATGACAACAGGA |
| | | CCCTCACTCTACTCAGTGTCACAAGGAATGATGTAGGACCCT |
| | | ATGAGTGTGGAATCCAGAACGAATTAAGTGTTGACCACAGC |
| | | GACCCAGTCATCCTGAATGTCCTCTATGGCCCAGACGACCCC |
| | | ACCATTTCCCCCTCATACACCTATTACCGTCCAGGGGTGAACC |
| | | TCAGCCTCTCCTGCCATGCAGCCTCTAACCCACCTGCACAGT |
| | | ATTCTTGGCTGATTGATGGGAACATCCAGCAACACACACAAG |
| | | AGCTCTTTATCTCCAACATCACTGAGAAGAACAGCGGACTCT |
| | | ATACCTGCCAGGCCAATAACTCAGCCAGTGGCCACAGCAGG |
| | | ACTACAGTCAAGACAATCACAGTCTCTGCGGAGCTGCCCAA |
| | | GCCCTCCATCTCCAGCAACAACTCCAAACCCGTGGAGGACA |
| | | AGGATGCTGTGGCCTTCACCTGTGAACCTGAGGCTCAGAAC |
| | | ACAACCTACCTGTGGTGGGTAAATGGTCAGAGCCTCCCAGTC |
| | | AGTCCCAGGCTGCAGCTGTCCAATGGCAACAGGACCCTCAC |
| | | TCTATTCAATGTCACAAGAAATGACGCAAGAGCCTATGTATG |
| | | TGGAATCCAGAACTCAGTGAGTGCAAACCGCAGTGACCCAG |
| | | TCACCCTGGATGTCCTCTATGGGCCGGACACCCCCATCATTTC |
| | | CCCCCCAGACTCGTCTTACCTTTCGGGAGCGAACCTCAACCT |
| | | CTCCTGCCACTCGGCCTCTAACCCATCCCCGCAGTATTCTTGG |
| | | CGTATCAATGGGATACCGCAGCAACACACACAAGTTCTCTTT |
| | | ATCGCCAAAATCACGCCAAATAATAACGGGACCTATGCCTGT |
| | | TTTGTCTCTAACTTGGCTACTGGCCGCAATAATTCCATAGTCA |
| | | AGAGCATCACAGTCTCTGCATCTGGAACTTCTCCTGGTCTCT |
| | | CAGCTGGGGCCACTGTCGGCATCATGATTGGAGTGCTGGTTG |
| | | GGGTTGCTCTGATATAG |
| SEQ ID | monkey | ATGGGGTCTCCCTCAGCCCCTCTTCACAGATGGTGCATCCCC |
| NO: 105 | CEA DNA | TGGCAGACGCTCCTGCTCACAGCCTCACTTCTAACCTTCTGG |
| | | AACCCGCCCACCACTGCCCAGCTCACTATTGAATCCAGGCCG |
| | | TTCAATGTTGCAGAGGGGAAGGAGGTTCTTCTACTTGCCCAC |
| | | AATGTGTCCCAGAATCTTTTTGGCTACATTTGGTACAAGGGA |
| | | GAAAGAGTGGATGCCAGCCGTCGAATTGGATCATGTGTAATA |
| | | AGAACTCAACAAATTACCCCAGGGCCCGCACACAGCGGTCG |
| | | AGAGACAATAGACTTCAATGCATCCCTGCTGATCCACAATGT |
| | | CACCCAGAGTGACACAGGATCCTACACCATACAAGTCATAAA |
| | | GGAAGATCTTGTGAATGAAGAAGCAACTGGCCAGTTCCGGG |
| | | TATACCCGGAGCTGCCCAAGCCCTACATCTCCAGCAACAACT |
| | | CCAACCCCGTGGAGGACAAGGATGCTGTGGCCTTAACCTGT |
| | | GAACCTGAGACTCAGGACACAACCTACCTGTGGTGGGTAAA |
| | | CAATCAGAGCCTCCCGGTCAGTCCCAGGCTGGAGCTGTCCA |
| | | GTGACAACAGGACCCTCACTGTATTCAATATTCCAAGAAATG |
| | | ACACAACATCCTACAAATGTGAAACCCAGAACCCAGTGAGT |
| | | GTCAGACGCAGCGACCCAGTCACCCTGAACGTCCTCTATGG |
| | | CCCGGATGCGCCCACCATTTCCCCTCTAAACACACCTTACAG |
| | | AGCAGGGGAAAATCTGAACCTCACCTGCCACGCAGCCTCTA |
| | | ACCCAACTGCACAGTACTTTTGGTTTGTCAATGGGACGTTCC |
| | | AGCAATCCACACAAGAGCTCTTTATACCCAACATCACCGTGA |
| | | ATAATAGCGGATCCTATATGTGCCAAGCCCATAACTCAGCCAC |
| | | TGGCCTCAATAGGACCACAGTCACGGCGATCACAGTCTACGC |
| | | GGAGCTGCCCAAGCCCTACATCACCAGCAACAACTCCAACC |
| | | CCATAGAGGACAAGGATGCTGTGACCTTAACCTGTGAACCTG |
| | | AGACTCAGGACACAACCTACCTGTGGTGGGTAAACAATCAG |
| | | AGCCTCTCGGTCAGTTCCAGGCTGGAGCTGTCCAATGACAA |
| | | CAGGACCCTCACTGTATTCAATATTCCAAGAAACGACACAAC |
| | | GTTCTACGAATGTGAGACCCAGAACCCAGTGAGTGTCAGAC |
| | | GCAGCGACCCAGTCACCCTGAATGTCCTCTATGGCCCGGATG |
| | | CGCCCACCATTTCCCCTCTAAACACACCTTACAGAGCAGGGG |
| | | AAAATCTGAACCTCTCCTGCCACGCAGCCTCTAACCCAGCTG |
| | | CACAGTACTCTTGGTTTGTCAATGGGACGTTCCAGCAATCCA |
| | | CACAAGAGCTCTTTATACCCAACATCACCGTGAATAATAGCG |
| | | GATCCTATATGTGCCAAGCCCATAACTCAGCCACTGGCCTCA |
| | | ATAGGACCACAGTCACGGCGATCACAGTCTATGTGGAGCTGC |
| | | CCAAGCCCTACATCTCCAGCAACAACTCCAACCCCATAGAGG |
| | | ACAAGGATGCTGTGACCTTAACCTGTGAACCTGTGGCTGAG |
| | | AACACAACCTACCTGTGGTGGGTAAACAATCAGAGCCTCTC |
| | | GGTCAGTCCCAGGCTGCAGCTCTCCAATGGCAACAGGATCCT |
| | | CACTCTACTCAGTGTCACACGGAATGACACAGGACCCTATGA |
| | | ATGTGGAATCCAGAACTCAGAGAGTGCAAAACGCAGTGACC |
| | | CAGTCACCCTGAATGTCACCTATGGCCCAGACACCCCCATCA |
| | | TATCCCCCCCAGACTTGTCTTACCGTTCGGGAGCAAACCTCA |
| | | ACCTCTCCTGCCACTCGGACTCTAACCCATCCCCGCAGTATTC |
| | | TTGGCTTATCAATGGGACACTGCGGCAACACACACAAGTTCT |
| | | CTTTATCTCCAAAATCACATCAAACAATAGCGGGGCCTATGCC |
| | | TGTTTTGTCTCTAACTTGGCTACCGGTCGCAATAACTCCATAG |
| | | TCAAGAACATCTCAGTCTCCTCTGGCGATTCAGCACCTGGAA |
| | | GTTCTGGTCTCTCAGCTAGGGCTACTGTCGGCATCATAATTGG |
| | | AATGCTGGTTGGGGTTGCTCTGATGTAG |
| SEQ ID | full-length | ATGGGACCCCCCTCAGCCCCTCCCTGCAGATTGCATGTCCCC |
| NO: 106 | human | TGGAAGGAGGTCCTGCTCACAGCCTCACTTCTAACCTTCTGG |
| | CEACAM | AACCCACCCACCACTGCCAAGCTCACTATTGAATCCACGCCG |
| | 6 DNA | TTCAATGTCGCAGAGGGGAAGGAGGTTCTTCTACTCGCCCAC |
| | | AACCTGCCCCAGAATCGTATTGGTTACAGCTGGTACAAAGGC |
| | | GAAAGAGTGGATGGCAACAGTCTAATTGTAGGATATGTAATA |
| | | GGAACTCAACAAGCTACCCCAGGGCCCGCATACAGTGGTCG |
| | | AGAGACAATATACCCCAATGCATCCCTGCTGATCCAGAACGT |
| | | CACCCAGAATGACACAGGATTCTATACCCTACAAGTCATAAA |
| | | GTCAGATCTTGTGAATGAAGAAGCAACCGGACAGTTCCATGT |
| | | ATACCCGGAGCTGCCCAAGCCCTCCATCTCCAGCAACAACTC |
| | | CAACCCCGTGGAGGACAAGGATGCTGTGGCCTTCACCTGTG |
| | | AACCTGAGGTTCAGAACACAACCTACCTGTGGTGGGTAAAT |
| | | GGTCAGAGCCTCCCGGTCAGTCCCAGGCTGCAGCTGTCCAA |
| | | TGGCAACATGACCCTCACTCTACTCAGCGTCAAAAGGAACG |
| | | ATGCAGGATCCTATGAATGTGAAATACAGAACCCAGCGAGTG |
| | | CCAACCGCAGTGACCCAGTCACCCTGAATGTCCTCTATGGCC |
| | | CAGATGTCCCCACCATTTCCCCCTCAAAGGCCAATTACCGTC |
| | | CAGGGGAAAATCTGAACCTCTCCTGCCACGCAGCCTCTAAC |
| | | CCACCTGCACAGTACTCTTGGTTTATCAATGGGACGTTCCAG |
| | | CAATCCACACAAGAGCTCTTTATCCCCAACATCACTGTGAAT |
| | | AATAGCGGATCCTATATGTGCCAAGCCCATAACTCAGCCACTG |
| | | GCCTCAATAGGACCACAGTCACGATGATCACAGTCTCTGGAA |
| | | GTGCTCCTGTCCTCTCAGCTGTGGCCACCGTCGGCATCACGA |
| | | TTGGAGTGCTGGCCAGGGTGGCTCTGATATAG |
| SEQ ID | 1-687 of | ATGGAGTCTCCCTCGGCCCCTCCCCACAGATGGTGCATCCCC |
| NO: 107 | huCEA | TGGCAGAGGCTCCTGCTCACAGCCTCACTTCTAACCTTCTGG |
| | DNA | AACCCGCCCACCACTGCCAAGCTCACTATTGAATCCACGCCG |
| | | TTCAATGTCGCAGAGGGGAAGGAGGTGCTTCTACTTGTCCAC |
| | | AATCTGCCCCAGCATCTTTTTGGCTACAGCTGGTACAAAGGT |
| | | GAAAGAGTGGATGGCAACCGTCAAATTATAGGATATGTAATA |
| | | GGAACTCAACAAGCTACCCCAGGGCCCGCATACAGTGGTCG |
| | | AGAGATAATATACCCCAATGCATCCCTGCTGATCCAGAACATC |
| | | ATCCAGAATGACACAGGATTCTACACCCTACACGTCATAAAG |
| | | TCAGATCTTGTGAATGAAGAAGCAACTGGCCAGTTCCGGGT |
| | | ATACCCGGAGCTGCCCAAGCCCTCCATCTCCAGCAACAACTC |
| | | CAAACCCGTGGAGGACAAGGATGCTGTGGCCTTCACCTGTG |
| | | AACCTGAGACTCAGGACGCAACCTACCTGTGGTGGGTAAAC |
| | | AATCAGAGCCTCCCGGTCAGTCCCAGGCTGCAGCTGTCCAAT |
| | | GGCAACAGGACCCTCACTCTATTCAATGTCACAAGAAATGAC |
| | | ACAGCAAGCTACAAATGTGAAACCCAGAACCCAGTGAGTGC |
| | | CAGGCGCAGTGATTCAGTCATCCTGAATGTCCTCTATGGCCC |
| | | GGATGCCCCCACCATTTCCCCTCTAAACACATCTTACAGATCA |
| | | GGGGAAAATCTGAACCTCTCCTGCCACGCAGCCTCTAACCC |
| | | ACCTGCACAGTACTCTTGGTTTGTCAATGGGACTTTCCAGCA |
| | | ATCCACCCAAGAGCTCTTTATCCCCAACATCACTGTGAATAAT |
| | | AGTGGATCCTATACGTGCCAAGCCCATAACTCAGACACTGGC |
| | | CTCAATAGGACCACAGTCACGACGATCACAGTCTATGCAGAG |
| | | CCACCCAAACCCTTCATCACCAGCAACAACTCCAACCCCGT |
| | | GGAGGATGAGGATGCTGTAGCCTTAACCTGTGAACCTGAGAT |
| | | TCAGAACACAACCTACCTGTGGTGGGTAAATAATCAGAGCCT |
| | | CCCGGTCAGTCCCAGGCTGCAGCTGTCCAATGACAACAGGA |
| | | CCCTCACTCTACTCAGTGTCACAAGGAATGATGTAGGACCCT |
| | | ATGAGTGTGGAATCCAGAACGAATTAAGTGTTGACCACAGC |
| | | GACCCAGTCATCCTGAATGTCCTCTATGGCCCAGACGACCCC |
| | | ACCATTTCCCCCTCATACACCTATTACCGTCCAGGGGTGAACC |
| | | TCAGCCTCTCCTGCCATGCAGCCTCTAACCCACCTGCACAGT |
| | | ATTCTTGGCTGATTGATGGGAACATCCAGCAACACACACAAG |
| | | AGCTCTTTATCTCCAACATCACTGAGAAGAACAGCGGACTCT |
| | | ATACCTGCCAGGCCAATAACTCAGCCAGTGGCCACAGCAGG |
| | | ACTACAGTCAAGACAATCACAGTCTCTGCGGAGCTGCCCAA |
| | | GCCCTCCATCTCCAGCAACAACTCCAAACCCGTGGAGGACA |
| | | AGGATGCTGTGGCCTTCACCTGTGAACCTGAGGCTCAGAAC |
| | | ACAACCTACCTGTGGTGGGTAAATGGTCAGAGCCTCCCAGTC |
| | | AGTCCCAGGCTGCAGCTGTCCAATGGCAACAGGACCCTCAC |
| | | TCTATTCAATGTCACAAGAAATGACGCAAGAGCCTATGTATG |
| | | TGGAATCCAGAACTCAGTGAGTGCAAACCGCAGTGACCCAG |
| | | TCACCCTGGATGTCCTCTATGGGCCGGACACCCCCATCATTTC |
| | | CCCCCCAGACTCGTCTTACCTTTCGGGAGCGAACCTCAACCT |
| | | CTCCTGCCACTCGGCCTCTAACCCATCCCCGCAGTATTCTTGG |
| | | CGTATCAATGGGATACCGCAGCAACACACACAAGTTCTCTTT |
| | | ATCGCCAAAATCACGCCAAATAATAACGGGACCTATGCCTGT |
| | | TTTGTCTCTAACTTGGCTACTGGCCGCAATAATTCCATAGTCA |
| | | AGAGCATCACAGTCTCTGCATCTGGAACTTCTCCTGGTCTCT |
| | | CAGCTGGGGCCCATCACCATCACCATCAC |
| SEQ ID | 1-690 of | ATGGGGTCTCCCTCAGCCCCTCTTCACAGATGGTGCATCCCC |
| NO: 108 | monkey | TGGCAGACGCTCCTGCTCACAGCCTCACTTCTAACCTTCTGG |
| | CEA DNA | AACCCGCCCACCACTGCCCAGCTCACTATTGAATCCAGGCCG |
| | | TTCAATGTTGCAGAGGGGAAGGAGGTTCTTCTACTTGCCCAC |
| | | AATGTGTCCCAGAATCTTTTTGGCTACATTTGGTACAAGGGA |
| | | GAAAGAGTGGATGCCAGCCGTCGAATTGGATCATGTGTAATA |
| | | AGAACTCAACAAATTACCCCAGGGCCCGCACACAGCGGTCG |
| | | AGAGACAATAGACTTCAATGCATCCCTGCTGATCCACAATGT |
| | | CACCCAGAGTGACACAGGATCCTACACCATACAAGTCATAAA |
| | | GGAAGATCTTGTGAATGAAGAAGCAACTGGCCAGTTCCGGG |
| | | TATACCCGGAGCTGCCCAAGCCCTACATCTCCAGCAACAACT |
| | | CCAACCCCGTGGAGGACAAGGATGCTGTGGCCTTAACCTGT |
| | | GAACCTGAGACTCAGGACACAACCTACCTGTGGTGGGTAAA |
| | | CAATCAGAGCCTCCCGGTCAGTCCCAGGCTGGAGCTGTCCA |
| | | GTGACAACAGGACCCTCACTGTATTCAATATTCCAAGAAATG |
| | | ACACAACATCCTACAAATGTGAAACCCAGAACCCAGTGAGT |
| | | GTCAGACGCAGCGACCCAGTCACCCTGAACGTCCTCTATGG |
| | | CCCGGATGCGCCCACCATTTCCCCTCTAAACACACCTTACAG |
| | | AGCAGGGGAAAATCTGAACCTCACCTGCCACGCAGCCTCTA |
| | | ACCCAACTGCACAGTACTTTTGGTTTGTCAATGGGACGTTCC |
| | | AGCAATCCACACAAGAGCTCTTTATACCCAACATCACCGTGA |
| | | ATAATAGCGGATCCTATATGTGCCAAGCCCATAACTCAGCCAC |
| | | TGGCCTCAATAGGACCACAGTCACGGCGATCACAGTCTACGC |
| | | GGAGCTGCCCAAGCCCTACATCACCAGCAACAACTCCAACC |
| | | CCATAGAGGACAAGGATGCTGTGACCTTAACCTGTGAACCTG |
| | | AGACTCAGGACACAACCTACCTGTGGTGGGTAAACAATCAG |
| | | AGCCTCTCGGTCAGTTCCAGGCTGGAGCTGTCCAATGACAA |
| | | CAGGACCCTCACTGTATTCAATATTCCAAGAAACGACACAAC |
| | | GTTCTACGAATGTGAGACCCAGAACCCAGTGAGTGTCAGAC |
| | | GCAGCGACCCAGTCACCCTGAATGTCCTCTATGGCCCGGATG |
| | | CGCCCACCATTTCCCCTCTAAACACACCTTACAGAGCAGGGG |
| | | AAAATCTGAACCTCTCCTGCCACGCAGCCTCTAACCCAGCTG |
| | | CACAGTACTCTTGGTTTGTCAATGGGACGTTCCAGCAATCCA |
| | | CACAAGAGCTCTTTATACCCAACATCACCGTGAATAATAGCG |
| | | GATCCTATATGTGCCAAGCCCATAACTCAGCCACTGGCCTCA |
| | | ATAGGACCACAGTCACGGCGATCACAGTCTATGTGGAGCTGC |
| | | CCAAGCCCTACATCTCCAGCAACAACTCCAACCCCATAGAGG |
| | | ACAAGGATGCTGTGACCTTAACCTGTGAACCTGTGGCTGAG |
| | | AACACAACCTACCTGTGGTGGGTAAACAATCAGAGCCTCTC |
| | | GGTCAGTCCCAGGCTGCAGCTCTCCAATGGCAACAGGATCCT |
| | | CACTCTACTCAGTGTCACACGGAATGACACAGGACCCTATGA |
| | | ATGTGGAATCCAGAACTCAGAGAGTGCAAAACGCAGTGACC |
| | | CAGTCACCCTGAATGTCACCTATGGCCCAGACACCCCCATCA |
| | | TATCCCCCCCAGACTTGTCTTACCGTTCGGGAGCAAACCTCA |
| | | ACCTCTCCTGCCACTCGGACTCTAACCCATCCCCGCAGTATTC |
| | | TTGGCTTATCAATGGGACACTGCGGCAACACACACAAGTTCT |
| | | CTTTATCTCCAAAATCACATCAAACAATAGCGGGGCCTATGCC |
| | | TGTTTTGTCTCTAACTTGGCTACCGGTCGCAATAACTCCATAG |
| | | TCAAGAACATCTCAGTCTCCTCTGGCGATTCAGCACCTGGAA |
| | | GTTCTGGTCTCTCAGCTAGGGCTCATCACCATCACCATCAC |
| SEQ ID | 1-320 of | ATGGGACCCCCCTCAGCCCCTCCCTGCAGATTGCATGTCCCC |
| NO: 109 | CEACAM | TGGAAGGAGGTCCTGCTCACAGCCTCACTTCTAACCTTCTGG |
| | 6 DNA | AACCCACCCACCACTGCCAAGCTCACTATTGAATCCACGCCG |
| | | TTCAATGTCGCAGAGGGGAAGGAGGTTCTTCTACTCGCCCAC |
| | | AACCTGCCCCAGAATCGTATTGGTTACAGCTGGTACAAAGGC |
| | | GAAAGAGTGGATGGCAACAGTCTAATTGTAGGATATGTAATA |
| | | GGAACTCAACAAGCTACCCCAGGGCCCGCATACAGTGGTCG |
| | | AGAGACAATATACCCCAATGCATCCCTGCTGATCCAGAACGT |
| | | CACCCAGAATGACACAGGATTCTATACCCTACAAGTCATAAA |
| | | GTCAGATCTTGTGAATGAAGAAGCAACCGGACAGTTCCATGT |
| | | ATACCCGGAGCTGCCCAAGCCCTCCATCTCCAGCAACAACTC |
| | | CAACCCCGTGGAGGACAAGGATGCTGTGGCCTTCACCTGTG |
| | | AACCTGAGGTTCAGAACACAACCTACCTGTGGTGGGTAAAT |
| | | GGTCAGAGCCTCCCGGTCAGTCCCAGGCTGCAGCTGTCCAA |
| | | TGGCAACATGACCCTCACTCTACTCAGCGTCAAAAGGAACG |
| | | ATGCAGGATCCTATGAATGTGAAATACAGAACCCAGCGAGTG |
| | | CCAACCGCAGTGACCCAGTCACCCTGAATGTCCTCTATGGCC |
| | | CAGATGTCCCCACCATTTCCCCCTCAAAGGCCAATTACCGTC |
| | | CAGGGGAAAATCTGAACCTCTCCTGCCACGCAGCCTCTAAC |
| | | CCACCTGCACAGTACTCTTGGTTTATCAATGGGACGTTCCAG |
| | | CAATCCACACAAGAGCTCTTTATCCCCAACATCACTGTGAAT |
| | | AATAGCGGATCCTATATGTGCCAAGCCCATAACTCAGCCACTG |
| | | GCCTCAATAGGACCACAGTCACGATGATCACAGTCTCTGGAC |
| | | ATCACCATCACCATCAC |
| SEQ ID | 1-78 of | ATGGAGTCTCCCTCGGCCCCTCCCCACAGATGGTGCATCCCC |
| NO: 110 | huCEA | TGGCAGAGGCTCCTGCTCACAGCCTCACTTCTAACCTTCTGG |
| | DNA | AACCCGCCCACCACTGCCAAGCTCACTATTGAATCCACGCCG |
| | | TTCAATGTCGCAGAGGGGAAGGAGGTGCTTCTACTTGTCCAC |
| | | AATCTGCCCCAGCATCTTTTTGGCTACAGCTGGTACAAAGGT |
| | | GAAAGAGTGGATGGCAACCGTCAAATTATAGGATATGTAATA |
| | | GGAACTCAACAAGCTACCCCAGGGCCCGCATACAGTGGTCG |
| | | AGAGATAATATACCCCAATGCATCCCTGCTGATCCAGAACATC |
| | | ATCCAGAAT |
| SEQ ID | 398-687 of | GAATTAAGTGTTGACCACAGCGACCCAGTCATCCTGAATGTC |
| NO: 111 | huCEA | CTCTATGGCCCAGACGACCCCACCATTTCCCCCTCATACACCT |
| | DNA | ATTACCGTCCAGGGGTGAACCTCAGCCTCTCCTGCCATGCAG |
| | | CCTCTAACCCACCTGCACAGTATTCTTGGCTGATTGATGGGA |
| | | ACATCCAGCAACACACACAAGAGCTCTTTATCTCCAACATCA |
| | | CTGAGAAGAACAGCGGACTCTATACCTGCCAGGCCAATAACT |
| | | CAGCCAGTGGCCACAGCAGGACTACAGTCAAGACAATCACA |
| | | GTCTCTGCGGAGCTGCCCAAGCCCTCCATCTCCAGCAACAAC |
| | | TCCAAACCCGTGGAGGACAAGGATGCTGTGGCCTTCACCTG |
| | | TGAACCTGAGGCTCAGAACACAACCTACCTGTGGTGGGTAA |
| | | ATGGTCAGAGCCTCCCAGTCAGTCCCAGGCTGCAGCTGTCC |
| | | AATGGCAACAGGACCCTCACTCTATTCAATGTCACAAGAAAT |
| | | GACGCAAGAGCCTATGTATGTGGAATCCAGAACTCAGTGAGT |
| | | GCAAACCGCAGTGACCCAGTCACCCTGGATGTCCTCTATGGG |
| | | CCGGACACCCCCATCATTTCCCCCCCAGACTCGTCTTACCTTT |
| | | CGGGAGCGAACCTCAACCTCTCCTGCCACTCGGCCTCTAACC |
| | | CATCCCCGCAGTATTCTTGGCGTATCAATGGGATACCGCAGCA |
| | | ACACACACAAGTTCTCTTTATCGCCAAAATCACGCCAAATAA |
| | | TAACGGGACCTATGCCTGTTTTGTCTCTAACTTGGCTACTGGC |
| | | CGCAATAATTCCATAGTCAAGAGCATCACAGTCTCTGCATCTG |
| | | GAACTTCTCCTGGTCTCTCAGCTGGGGCC |
| SEQ ID | CEA-v | ATGGAGTCTCCCTCGGCCCCTCCCCACAGATGGTGCATCCCC |
| NO: 112 | DNA | TGGCAGAGGCTCCTGCTCACAGCCTCACTTCTAACCTTCTGG |
| | | AACCCGCCCACCACTGCCAAGCTCACTATTGAATCCACGCCG |
| | | TTCAATGTCGCAGAGGGGAAGGAGGTGCTTCTACTTGTCCAC |
| | | AATCTGCCCCAGCATCTTTTTGGCTACAGCTGGTACAAAGGT |
| | | GAAAGAGTGGATGGCAACCGTCAAATTATAGGATATGTAATA |
| | | GGAACTCAACAAGCTACCCCAGGGCCCGCATACAGTGGTCG |
| | | AGAGATAATATACCCCAATGCATCCCTGCTGATCCAGAACATC |
| | | ATCCAGAATGAATTAAGTGTTGACCACAGCGACCCAGTCATC |
| | | CTGAATGTCCTCTATGGCCCAGACGACCCCACCATTTCCCCC |
| | | TCATACACCTATTACCGTCCAGGGGTGAACCTCAGCCTCTCC |
| | | TGCCATGCAGCCTCTAACCCACCTGCACAGTATTCTTGGCTG |
| | | ATTGATGGGAACATCCAGCAACACACACAAGAGCTCTTTATC |
| | | TCCAACATCACTGAGAAGAACAGCGGACTCTATACCTGCCAG |
| | | GCCAATAACTCAGCCAGTGGCCACAGCAGGACTACAGTCAA |
| | | GACAATCACAGTCTCTGCGGAGCTGCCCAAGCCCTCCATCTC |
| | | CAGCAACAACTCCAAACCCGTGGAGGACAAGGATGCTGTGG |
| | | CCTTCACCTGTGAACCTGAGGCTCAGAACACAACCTACCTGT |
| | | GGTGGGTAAATGGTCAGAGCCTCCCAGTCAGTCCCAGGCTG |
| | | CAGCTGTCCAATGGCAACAGGACCCTCACTCTATTCAATGTC |
| | | ACAAGAAATGACGCAAGAGCCTATGTATGTGGAATCCAGAA |
| | | CTCAGTGAGTGCAAACCGCAGTGACCCAGTCACCCTGGATG |
| | | TCCTCTATGGGCCGGACACCCCCATCATTTCCCCCCCAGACT |
| | | CGTCTTACCTTTCGGGAGCGAACCTCAACCTCTCCTGCCACT |
| | | CGGCCTCTAACCCATCCCCGCAGTATTCTTGGCGTATCAATGG |
| | | GATACCGCAGCAACACACACAAGTTCTCTTTATCGCCAAAAT |
| | | CACGCCAAATAATAACGGGACCTATGCCTGTTTTGTCTCTAAC |
| | | TTGGCTACTGGCCGCAATAATTCCATAGTCAAGAGCATCACA |
| | | GTCTCTGCATCTGGAACTTCTCCTGGTCTCTCAGCTGGGGCC |
| | | CATCACCATCACCATCAC |
| SEQ ID | 1-273 of | ATGGGACCCCCCTCAGCCCCTCCCTGCAGATTGCATGTCCCC |
| NO: 113 | NCA DNA | TGGAAGGAGGTCCTGCTCACAGCCTCACTTCTAACCTTCTGG |
| | | AACCCACCCACCACTGCCAAGCTCACTATTGAATCCACGCCG |
| | | TTCAATGTCGCAGAGGGGAAGGAGGTTCTTCTACTCGCCCAC |
| | | AACCTGCCCCAGAATCGTATTGGTTACAGCTGGTACAAAGGC |
| | | GAAAGAGTGGATGGCAACAGTCTAATTGTAGGATATGTAATA |
| | | GGAACTCAACAAGCTACCCCAGGGCCCGCATACAGTGGTCG |
| | | AGAGACAATATACCCCAATGCATCCCTGCTGATCCAGAACGT |
| | | CACCCAGAATGACACAGGATTCTATACCCTACAAGTCATAAA |
| | | GTCAGATCTTGTGAATGAAGAAGCAACCGGACAGTTCCATGT |
| | | ATACCCGGAGCTGCCCAAGCCCTCCATCTCCAGCAACAACTC |
| | | CAACCCCGTGGAGGACAAGGATGCTGTGGCCTTCACCTGTG |
| | | AACCTGAGGTTCAGAACACAACCTACCTGTGGTGGGTAAAT |
| | | GGTCAGAGCCTCCCGGTCAGTCCCAGGCTGCAGCTGTCCAA |
| | | TGGCAACATGACCCTCACTCTACTCAGCGTCAAAAGGAACG |
| | | ATGCAGGATCCTATGAATGTGAAATACAGAACCCAGCGAGTG |
| | | CCAACCGCAGTGACCCAGTCACCCTGAATGTCCTCTATGGCC |
| | | CAGATGTCCCCACCATTTCC |
| SEQ ID | 596-687 of | CCCATCATTTCCCCCCCAGACTCGTCTTACCTTTCGGGAGCG |
| NO: 114 | huCEA | AACCTCAACCTCTCCTGCCACTCGGCCTCTAACCCATCCCCG |
| | DNA | CAGTATTCTTGGCGTATCAATGGGATACCGCAGCAACACACA |
| | | CAAGTTCTCTTTATCGCCAAAATCACGCCAAATAATAACGGG |
| | | ACCTATGCCTGTTTTGTCTCTAACTTGGCTACTGGCCGCAATA |
| | | ATTCCATAGTCAAGAGCATCACAGTCTCTGCATCTGGAACTT |
| | | CTCCTGGTCTCTCAGCTGGGGCC |
| SEQ ID | CHIM | ATGGGACCCCCCTCAGCCCCTCCCTGCAGATTGCATGTCCCC |
| NO: 115 | DNA | TGGAAGGAGGTCCTGCTCACAGCCTCACTTCTAACCTTCTGG |
| | | AACCCACCCACCACTGCCAAGCTCACTATTGAATCCACGCCG |
| | | TTCAATGTCGCAGAGGGGAAGGAGGTTCTTCTACTCGCCCAC |
| | | AACCTGCCCCAGAATCGTATTGGTTACAGCTGGTACAAAGGC |
| | | GAAAGAGTGGATGGCAACAGTCTAATTGTAGGATATGTAATA |
| | | GGAACTCAACAAGCTACCCCAGGGCCCGCATACAGTGGTCG |
| | | AGAGACAATATACCCCAATGCATCCCTGCTGATCCAGAACGT |
| | | CACCCAGAATGACACAGGATTCTATACCCTACAAGTCATAAA |
| | | GTCAGATCTTGTGAATGAAGAAGCAACCGGACAGTTCCATGT |
| | | ATACCCGGAGCTGCCCAAGCCCTCCATCTCCAGCAACAACTC |
| | | CAACCCCGTGGAGGACAAGGATGCTGTGGCCTTCACCTGTG |
| | | AACCTGAGGTTCAGAACACAACCTACCTGTGGTGGGTAAAT |
| | | GGTCAGAGCCTCCCGGTCAGTCCCAGGCTGCAGCTGTCCAA |
| | | TGGCAACATGACCCTCACTCTACTCAGCGTCAAAAGGAACG |
| | | ATGCAGGATCCTATGAATGTGAAATACAGAACCCAGCGAGTG |
| | | CCAACCGCAGTGACCCAGTCACCCTGAATGTCCTCTATGGCC |
| | | CAGATGTCCCCACCATTTCCCCCATCATTTCCCCCCCAGACTC |
| | | GTCTTACCTTTCGGGAGCGAACCTCAACCTCTCCTGCCACTC |
| | | GGCCTCTAACCCATCCCCGCAGTATTCTTGGCGTATCAATGGG |
| | | ATACCGCAGCAACACACACAAGTTCTCTTTATCGCCAAAATC |
| | | ACGCCAAATAATAACGGGACCTATGCCTGTTTTGTCTCTAACT |
| | | TGGCTACTGGCCGCAATAATTCCATAGTCAAGAGCATCACAG |
| | | TCTCTGCATCTGGAACTTCTCCTGGTCTCTCAGCTGGGGCCC |
| | | ATCACCATCACCATCAC |

### Example 2. Cloning and Sequence Analysis of CEA Antibodies

Murine hybridoma cells were harvested to prepare total RNAs using Ultrapure RNA kit (Cat. No. 74104, QIAGEN, Germany) based on the manufacturer's protocol. The 1S′ strand cDNAs were synthesized using a cDNA synthesis kit from Invitrogen (Cat. No. 18080-051) and PCR amplification of VH and VL genes of murine monoclonal antibodies was performed using a PCR kit (Cat. No. CW0686, CWBio, Beijing, China). The oligo primers used for antibody cDNAs cloning of heavy chain variable region (VH) and kappa light chain variable region (VL) were synthesized based on the sequences reported previously (Brocks et al., Mol Med. 2001 7(7):461-9.). PCR products were then subcloned into the pEASY-Blunt cloning vector (Cat. No. CB101-02, TransGen, China) and sequenced. The amino acid sequences of VH and VL regions were determined from the DNA sequencing results.

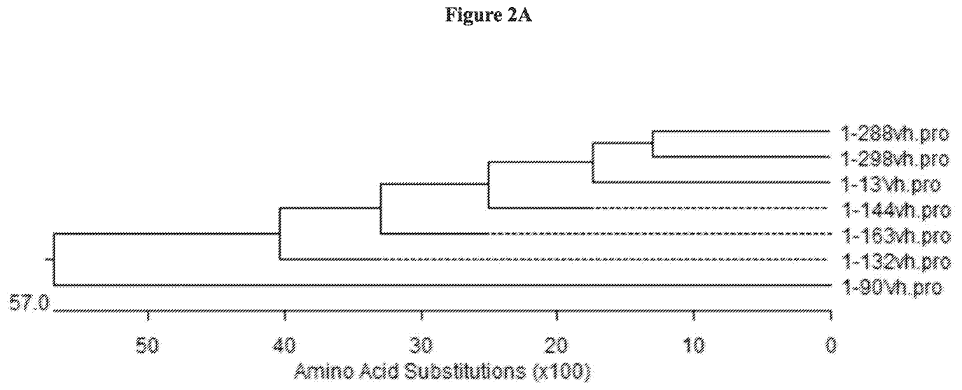

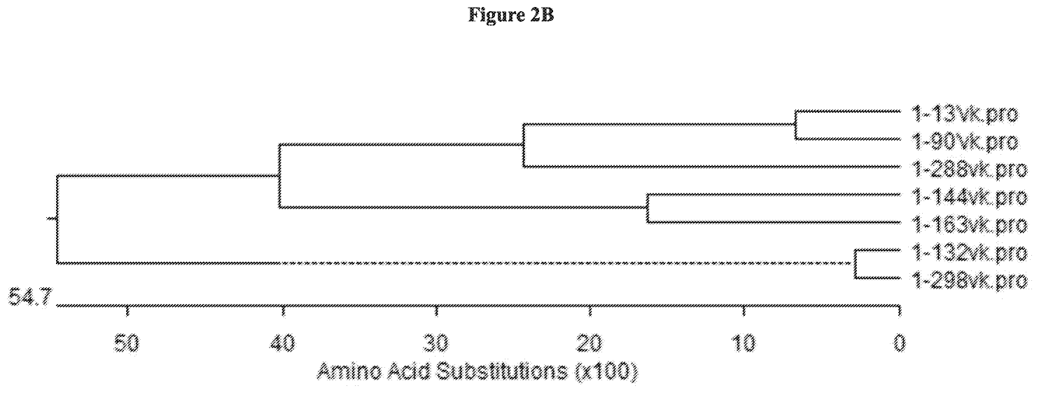

The monoclonal antibodies were analyzed by comparing sequence homology and grouped based on sequence similarity (FIG. 2). Complementary determinant regions (CDRs) were defined based on the IMGT (Lefranc et al., 1999 Nucleic Acids Research 27:209-212) system by sequence annotation. The amino acid sequences of a representative clone BGA13 are listed in Table 4.

TABLE 4
amino acid sequences

| | | | |
|---|---|---|---|
| BGA13 | SEQ ID | HCDR1 | GYIFTSYW |
| (IMGT) | NO: 116 | (IMGT) | |
| | SEQ ID | HCDR2 | INPNTGYT |
| | NO: 117 | (IMGT) | |
| | SEQ ID | HCDR3 | AREYGNYNYPLDY |
| | NO: 3 | (IMGT) | |
| | SEQ ID | LCDR1 | ENIYGY |
| | NO: 118 | (IMGT) | |
| | SEQ ID | LCDR2 | NA |
| | NO: 119 | (IMGT) | |
| | SEQ ID | LCDR3 | QHHYGTPYT |
| | NO: 120 | (IMGT) | |
| BGA13 | SEQ ID | VH | QVQLQQSGAELAKSGASVKMSCKASGYIFT |
| | NO: 121 | | SYWLHWVKQRPGQGLEWIGYINPNTGYTNY |
| | | | SQKFKDKATLTADKSSSTAYMQLSSLTSEDSA |
| | | | VYYCAREYGNYNYPLDYWGQGTSVTVSS |
| | SEQ ID | VL | DIQMTQSPASLSASVGETVTITCRASENIYGY |
| | NO: 122 | | LAWYQQKQGKSPQLLVFNAKNLVEGVPSRF |
| | | | SGSGSGTQFSLKINSLQPEDFGSYYCQHHYG |
| | | | TPYTFGGGTKLEIK |

### Example 3. Binding Profiles Determination of Purified Murine Anti-CEA Antibodies

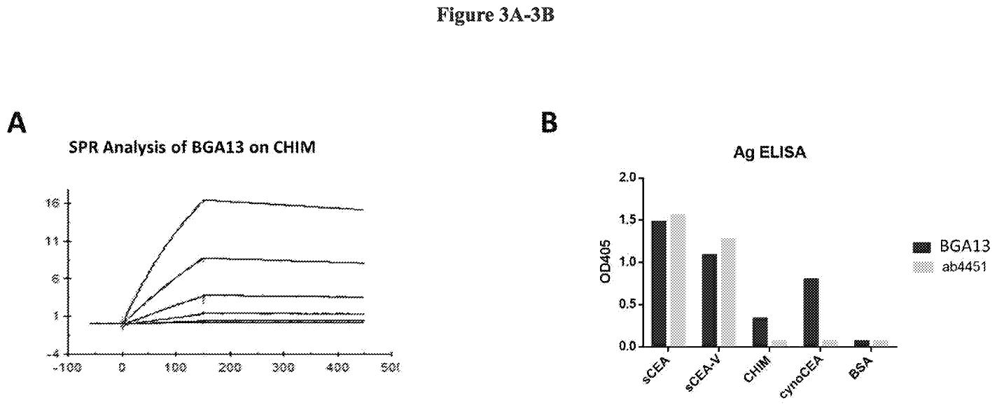

The CEA antibodies with specific binding for CEA as shown by ELISA and FACS, as well as without soluble CEA (sCEA) interference were characterized for their binding kinetics by SPR assays using BIAcore™ T-200 (GE Life Sciences) (FIG. 3A). Briefly, anti-murine IgG antibody was immobilized on an activated CM5 biosensor chip (Cat. No. BR100530, GE Life Sciences). Purified murine antibodies were flowed over the chip surface and captured by anti-murine IgG antibody. Then a serial dilution (6.0 nM to 2150 nM) of purified CHIM, CEA-v, CEA or monkey CEA recombinant proteins were flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The binding affinity profiles of BGA13 is shown below in Table 5.

The binding profiles of BGA13 were checked via antigen ELISA, the bindings of purified BGA13 to huCEA and monkey CEA were observed, these indicated BGA13 is a weak binder to soluble huCEA and monkey CEA, or soluble CEA has a different conformation when immobilized (FIG. 3B). For this experiment, sCEA, CHIM, monkey CEA, CEA-v and BSA were coated in 96-well plates at a high concentration of 10 μg/ml overnight at 4° C. BGA13 or a control antibody ab4451 (Cat. No. ab4451, abcam, USA) were incubated for 1 hour at a concentration of 2 μg/ml. The HRP-linked anti-mouse IgG antibody (Cat. No. 7076S, Cell Signaling Technology, USA) and substrate (Cat. No. 00-4201-56, eBioscience, USA) were used for development, and absorbance signal at the wavelength of 450 nm was measured using a plate reader (SpectraMax Paradigm, Molecular Devices, USA).

TABLE 5
Comparison of BGA13 binding affinities by SPR

| Antigen | KD (M) |
|---|---|
| sCEA | ND |
| sCEA-v | 1.50E−07 |
| CHIM | 1.20E−07 |
| Monkey CEA | ND (not detectable, due to weak binding) |

### Example 4. Effects of Recombinant Soluble CEA on Binding of BGA13 to CEA Expressing Cells

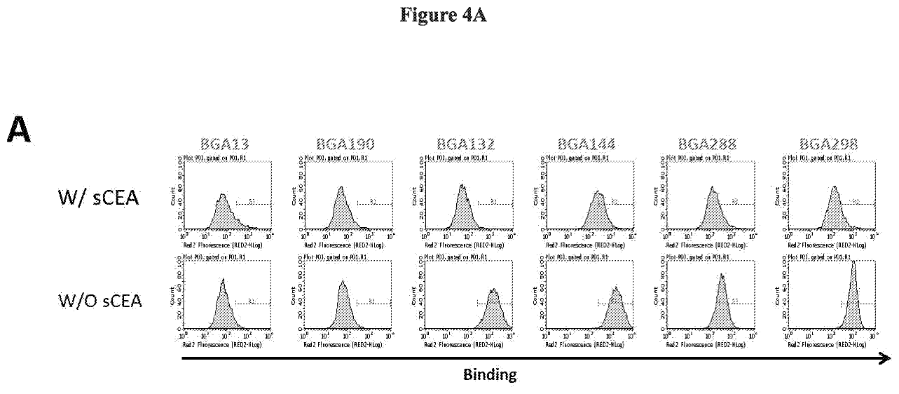

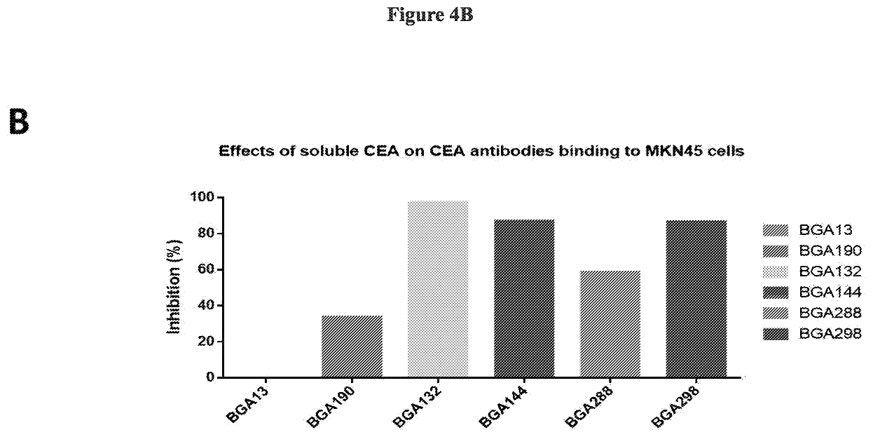

The presence of soluble CEA on the specific binding of various CEA antibodies to CEA expressing cells was evaluated via flow cytometry. In brief, human CEA-expressing cells (10⁵ cells/well) were incubated with 2 μg/ml purified CEA murine monoclonal antibodies in the presence of 20 μg/ml extra recombinant soluble CEA proteins, followed by binding with Alexa Fluro-647-labeled goat anti-mouse IgG antibody (Cat. No. A0473, Beyotime Biotechnology, China). Cell fluorescence was quantified using a flow cytometer (Guava easyCyte™ 8HT, Merck-Millipore, USA). As shown in FIGS. 4A and 4B, the binding of BGA13 to CEA expressing cells were not affected by the presence of soluble CEA.

### Example 5. Humanization of the Murine Anti-Human CEA Antibodies mAb Humanization and Engineering

For humanization of BGA13, human germline IgG genes were searched for sequences that share high degrees of homology with the cDNA sequences of BGA13 variable regions by sequence comparisons in the human immunoglobulin gene databases at IMGT and NCBI. The human IGVH and IGVL genes that are present in human antibody repertoires with high frequencies (Glanville et al., 2009 PNAS 106:20216-20221) and are highly homologous to BGA13 were selected as the templates for humanization. Before humanization, BGA13 heavy and light chain variable domains were fused to a wild type human IgG1 constant region designated as human IgG1 wt (SEQ ID NO:123) and a human kappa constant (CL) region (SEQ ID NO:124), respectively.

TABLE 6
amino acid sequences

| | | |
|---|---|---|
| SEQ ID | ASTKGPSVFPLAPSSKSTSGGTAALGCLVKDYFPEPVTVSWNSGALTS | IgG1wt |
| NO: 123 | GVHTFPAVLQSSGLYSLSSVVTVPSSSLGTQTYICNVNHKPSNTKVDK | |
| | KVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLT | |
| | VLHQDWLNGKEYKCKVSNKALPAPIEKTISKAKGQPREPQVYTLPPS | |
| | RDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPGK | |
| SEQ ID | RTVAAPSVFIFPPSDEQLKSGTASVVCLLNNFYPREAKVQWKVDNAL | CL |
| NO: 124 | QSGNSQESVTEQDSKDSTYSLSSTLTLSKADYEKHKVYACEVTHQGL | |
| | SSPVTKSFNRGEC | |

Humanization was carried out by CDR-grafting (Methods in Molecular Biology, Vol 248: Antibody Engineering, Methods and Protocols, Humana Press) and the BGA13 antibody was engineered in the human IgG1 format. In the initial round of humanization, mutations from murine to human amino acid residues in framework regions were guided by the simulated 3D structure, and the murine framework residues of structural importance for maintaining the canonical structures of CDRs were retained in the 1^{st} version of humanized antibody BGA13, BGA131 (the amino acid sequences of the heavy chain and light chain are set forth in SEQ ID NOs: 125 and 126).

TABLE 7
amino acid sequences

| | | |
|---|---|---|
| SEQ ID | QVQLVQSGAEVKKPGASVKVSCKASGYIFTSYWLHWVRQAPGQGL | BGA131 |
| NO: 125 | EWIGYINPNTGYTNYSQKFQGRVTMTRDTSTSTVYMELSSLRSEDTA | heavy |
| | VYYCAREYGNYNYPLDYWGQGTLVTVSSASTKGPSVFPLAPSSKSTS | chain |
| | GGTAALGCLVKDYFPEPVTVSWNSGALTSGVHTFPAVLQSSGLYSLSS | |
| | VVTVPSSSLGTQTYICNVNHKPSNTKVDKKVEPKSCDKTHTCPPCPA | |
| | PELLGGPSVFLFPPKPKDTLMISRTPEVTCVVVDVSHEDPEVKFNWY | |
| | VDGVEVHNAKTKPREEQYNSTYRVVSVLTVLHQDWLNGKEYKCKV | |
| | SNKALPAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQ | |
| | GNVFSCSVMHEALHNHYTQKSLSLSPGK | |
| SEQ ID | DIQMTQSPSSLSASVGDRVTITCRASENIYGYLAWYQQKPGKVPKLLI | BGA131 |
| NO: 126 | YNAKNLVEGVPSRFSGSGSGTDFTLTISSLQPEDVATYYCQHHYGTPY | light |
| | TFGQGTKVEIKRTVAAPSVFIFPPSDEQLKSGTASVVCLLNNFYPREA | chain |
| | KVQWKVDNALQSGNSQESVTEQDSKDSTYSLSSTLTLSKADYEKHK | |
| | VYACEVTHQGLSSPVTKSFNRGEC | |

Specifically, CDRs of BGA13 VL were grafted into the frameworks of human germline variable gene IGVK1-27 with 2 murine framework residues (N66 and V68) retained (the amino acid sequence of the light chain variable domain is set forth in SEQ ID NO: 128). CDRs of BGA13 VH were grafted into the frameworks of human germline variable gene IGVH1-46 with 5 murine framework (L39, 153, Y55, N66, S68) residues retained (the amino acid sequence of the heavy chain variable domain is set for in SEQ ID NO: 127).

TABLE 8
amino acid sequences

| | | |
|---|---|---|
| SEQ ID | QVQLVQSGAEVKKPGASVKVSCKASGYIFTSYWLHWVRQAPGQGL | BGA131 |
| NO: 127 | EWIGYINPNTGYTNYSQKFQGRVTMTRDTSTSTVYMELSSLRSEDTA | VH |
| | VYYCAREYGNYNYPLDYWGQGTLVTVSS | |
| SEQ ID | DIQMTQSPSSLSASVGDRVTITCRASENIYGYLAWYQQKPGKVPKLLI | BGA131 |
| NO: 128 | YNAKNLVEGVPSRFSGSGSGTDFTLTISSLQPEDVATYYCQHHYGTPY | VL |
| | TFGQGTKVEIK | |

BGA13-1 was constructed as human full-length antibody format using in-house developed expression vectors that contain constant regions of a wild type human IgG1 with easy adapting subcloning sites. Expression and preparation of BGA13-1 antibody was achieved by co-transfection of the above two constructs into 293G cells and by purification using a Protein A column (Cat. No. 17543802, GE Life Sciences). The purified antibodies were concentrated to 0.5-5 mg/mL in PBS and stored in aliquots in −80° C. freezer.

Using BGA131, an additional number of single or multiple amino acid changes were made, converting the human residues in framework regions of VH and VL to corresponding murine germline residues, which include V68A, R72A and V79A in VH and V43S in VL, respectively. This resulted BGA132 (V68A, R72A in VH), BGA133 (V79A in VH), BGA134 (V68A, R72A, V79A in VH), BGA135 (V43S in VL), BGA136 (V68A, R72A in VH, and V43S in VL), BGA137 (V79A in VH, V43S in VL) and BGA138 (V68A, R72A, V79A, in VH and V43S in VL). All antibodies which contained modifications had similar binding activities to BGA131, and none of the changes abolished binding.

In order to remove post-translational modification (PTM) sites, further engineering was made by introducing mutations in CDRs and framework regions based on the BGA131 sequence, which include N52T, N54Q, N59S, N102G, N104Q and S61A amino acid changes in the VH region. This resulted in BGA131A (N52T (VH)), BGA131B (N54Q (VH)), BGA131C (N59S (VH)), BGA131D (N102G (VH)), BGA131E (N104Q (VH)) and BGA131F (N54Q, N59S, S61A (VH)) and all of the antibodies had similar binding specificity to BGA131, with none of the changes abolishing binding. While maintaining specificity, amino acid compositions and expression levels were also considered. All humanization mutations were made using primers containing mutations at specific positions and a site directed mutagenesis kit (Cat. FM111-02, TransGen, Beijing, China). The desired mutations were verified by sequence analysis. Comparing to BGA13-1, BGA13-1F had significantly reduced binding affinities with no glycosylation sites but had a high expression level (Table 9).

TABLE 9
amino acid sequences

| | | |
|---|---|---|
| SEQ ID | DIQMTQSPSSLSASVGDRVTITCRASENIYGYLAWYQQKPGKVPKLLI | BGA13-1 |
| NO: 129 | YNAKNLVEGVPSRFSGSGSGTDFTLTISSLQPEDVATYYCQHHYGTPY | VL |
| | TFGQGTKVEIK | |
| SEQ ID | QVQLVQSGAEVKKPGASVKVSCKASGYIFTSYWLHWVRQAPGQGL | BGA13-1F |
| NO: 130 | EWIGYINPQTGYTSYAQKFQGRVTMTRDTSTSTVYMELSSLRSEDTA | VH |
| | VYYCAREYGNYNYPLDYWGQGTLVTVSS | |

### Example 6. Generation of Affinity Maturation Libraries

A phagemid vector pCANTAB 5E (GE Healthcare) was used by standard molecular biology techniques to construct a phagemid designed to display BGA13-1F Fab fragments on the surface of M13 bacteriophage as a fusion with the N-terminus of a fragment of the gene-3 minor coat protein. There was an amber stop codon before the g3 sequence to allow expression of Fab fragments directly from phagemid clones. The phagemid was used as the template to construct phage-displayed libraries containing 108 unique members.

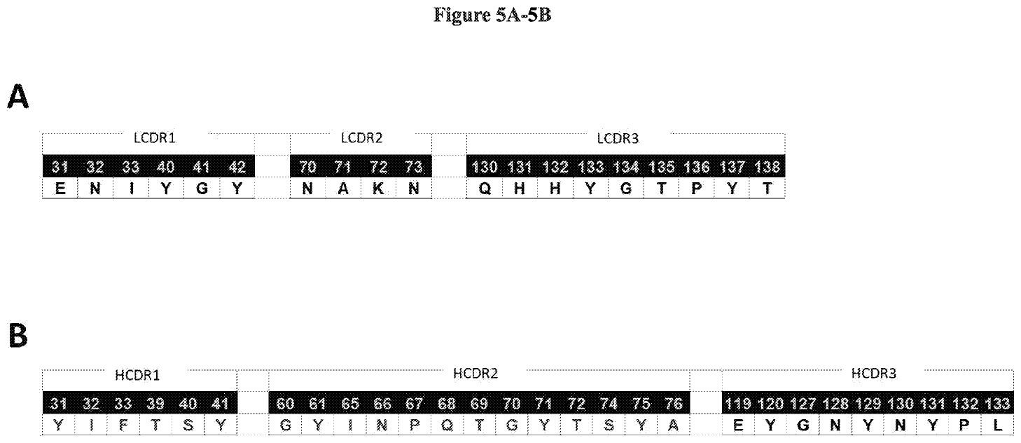

Two libraries (H-AM, L-AM) were constructed randomizing CDR positions in the heavy and light chains, respectively. All three CDRs were randomized in each library but each CDR had a maximum of one mutation in each clone except HCDR3, which could have two simultaneous mutations. Each position was randomized with an NNK codon (IUPAC code) encoding any amino acid or an amber stop codon. The combined heavy and light chain library designs had a potential diversity of 5.0×10⁶ unique full-length clones without stop or cysteine codons and an expected distribution of about 0.02%, 1.1%, 17% and 82% of clones with 0, 1, 2, and 3 mutations, respectively. A minor fraction of heavy chain clones was expected to have 4 mutations due to primer design in the HCDR3 region. As a first step, a DNA fragment was amplified using pCANTAB 5E as a template and primers which contains the randomized CDR3 positions (see FIGS. 5A and 5B). Then the PCR products were gel-purified and assembled with the primers which contains the randomized CDR2 positions. The procedure was repeated with the primers directed to random CDR1 positions. The resulting PCR products for heavy chain or light chain were then assembled with its corresponding CH fragment or CL fragment by overlapping PCR. The fragments were further assembled with the light chain or heavy chain with no mutations by overlapping PCR. The resulting fragments were then gel-purified and ligated with pCANTAB 5E after NcoI/NotI digestion. The purified ligations were transformed into TG1 bacteria by electroporation. Sequencing of 48 clones from each library confirmed the randomization of each position (data not shown), although not all amino acid mutations were observed in every position due to the limited sampling depth. About 52% and 55% of the light and heavy chain libraries had full-length randomized clones, enough to cover all the potential diversity of the design with the 108 independent clones generated even with moderate incorporation biases in oligonucleotide synthesis and library construction.

### Example 7. Generation of Affinity Matured Humanized BGA13 Variants

### Library Selection and Screening

Generation of affinity-matured humanized BGA13 Fabs was carried out by phage display using standard protocols (Silacci et al., (2005) Proteomics, 5, 2340-50; Zhao et al., (2014) PLoS One, 9, e111339). For the first and second rounds of selections, competition selections were performed on immobilized CHIM in immune tubes (Cat. No. 470319, ThermoFisher). In brief, immunotubes were coated with 1 ml of CHIM (5 μg/ml in PBS) overnight at 4° C. All affinity maturation libraries were incubated with the coated immunotubes for 1 hour in the presence of various concentrations of BGA13-1F IgG (round 1, 1 μg/ml; round 2, 5 μg/ml). For the third and fourth rounds of selections, cell panning was carried out using L929/huCEA cells (round 3) or LOVO cells (ATCC CCL-229) (round 4) with HEK293 cells as depletion cells. After four rounds of selections, individual clones were picked up and phage containing supernatants were prepared using standard protocols. ELISA-positive clones were sequenced, and mutation sites were analyzed.

### Analysis of Mutation Frequency in CDRs

The frequency of mutations in each CDR after four rounds of selection was relatively high, ranging from 17% in HCDR3 to 95% in LCDR2. Regarding the heavy chain, about half of clones identified in H-AM library were identical to the parental clone. The other clones contained one back-mutation at Q54N in HCDR2.

In analyzing the light chain, the mutations were much more diverse. Two sites had mutations occurring in almost all of clones in LCDR1, respectively. Light chain residues 29 and 31 were mutated from Ile to Gln and Gly to Gln in 47.09% and 35.29% of the clones, respectively. Position 29 not only had a high frequency of Gln mutation, but also had a subset of clones with a mutation to Tyrosine. Position 31 not only had a high frequency of Gln mutation, but also had about 12.5% chance to be mutated to Leu. Due to library design constraints, mutations in positions 29 and 31 were not found in combination with each other. However, mutations in each of these two sites were often combined with mutations in other CDRs. Regarding LCDR2, only A51 had mutations occurring in at least 64.71% clones, but with not any obvious pattern, which included large, hydrophobic and polar residues, such as Tyr, Phe, Thr and Asn. Regarding to LCDR3, two sites had mutations occurring in at least 50% clones. Light chain residues 90 and 92 were mutated form His to Leu and Tyr to Leu in 11.76% and 47.06% of the clones, respectively. FIG. 6 shows the sequence variance of CDR regions of light chain after four rounds of selections.

### Expression of Selected Humanized BGA13 Variants

Combination of mutations were made. Light chain variable regions from selected phage clones were subcloned into a human kappa light chain expression mammalian expression vector. The light chain expression vectors were co-transfected into 293G cells with a mammalian expression vector expressing BGA13-1F heavy chain at a 1:1 ratio. Versions of CEA antibodies were purified from culture supernatants by Protein A affinity chromatography (Cat. No. 17543802, GE Life Sciences). The purified antibodies were concentrated to 0.5-5 mg/mL in PBS and stored in aliquots in −80° C. freezer.

### Characterization of Affinity Matured Humanized BGA13 Variants

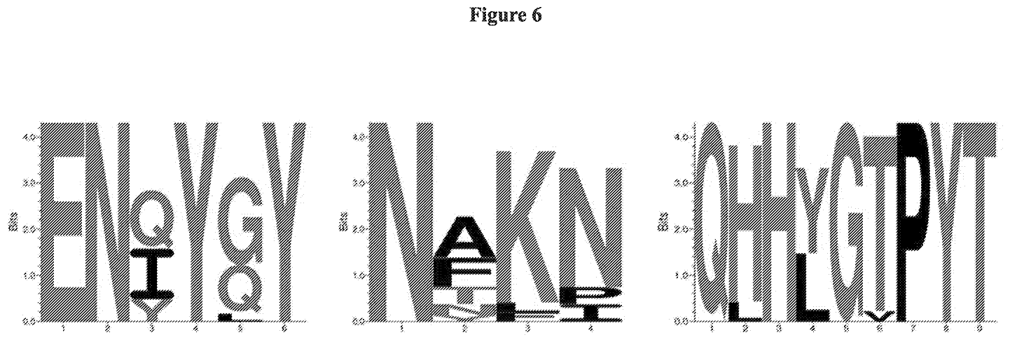

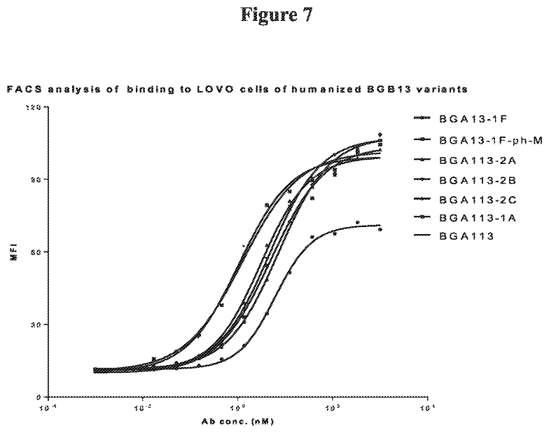

Affinity comparison of BGA13-1F and other affinity matured clones was made by SPR assay (Table 10) using BIAcore™ T-200 (GE Life Sciences) and flow cytometry (FIG. 7). For this experiment, anti-human IgG (Fc) antibody was immobilized on an activated CM5 biosensor chip (Cat. No. BR100839, GE Life Sciences). Anti-CEA antibodies were flowed over the chip surface and captured by anti-human Fab antibody. Then a serial dilution (1.37 nM to 333 nM) of CHIM was flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). For flow cytometry, CEA-expressing cells (10⁵ cells/well) were incubated with various concentrations of purified affinity-matured antibodies, followed by binding with Alexa Fluro-647-labeled anti-hu IgG Fc antibody (Cat. No. 409320, BioLegend, USA). Cell fluorescence was quantified using a flow cytometer (Guava easyCyte™ 8HT, Merck-Millipore, USA). The equilibrium dissociation constant (K_{D}) was calculated as the ratio k_{off}/kₒₙ. The BGA131F-ph-L (SEQ ID NO: 131) and BGA131F-ph-M (SEQ ID NO: 132) were shown with improved affinities to huCEA surface protein (Table 11).

TABLE 10
Comparison of binding affinities by SPR

| Sample ID | Ka(1/Ms) | Kd(1/s) | KD(M) | Rmax(RU) |
|---|---|---|---|---|
| 1F-ph-E | 3.4E+3 | 3.5E−3 | 1.0E−6 | 284 |
| 1F-ph-F | 1.0E+2 | 1.2E−2 | 1.2E−4 | 4162 |
| 1F-ph-G | 6.4E+1 | 2.6E−3 | 4.1E−5 | 3487 |
| 1F-ph-H | 1.1E+2 | 2.8E−3 | 2.5E−5 | 1983 |
| 1F-ph-I | 2.0E+2 | 2.2E−3 | 1.1E−5 | 2248 |
| 1F-ph-L | 6.7E+3 | 3.0E−4 | 4.6E−8 | 184 |
| 1F-ph-M | 2.5E+3 | 2.5E−4 | 9.9E−8 | 224 |
| 1F-ph-N | 1.1E+3 | 1.0E−2 | 9.2E−6 | 1267 |
| BGA13-1F | 1.9E+3 | 1.5E−3 | 7.9E−7 | 367 |

TABLE 11
amino acid sequences

| | | |
|---|---|---|
| SEQ ID | DIQMTQSPSSLSASVGDRVTITCRASENQYGYLAWYQQKPGKVPKLL | BGA13- |
| NO: 131 | IYNFKNLVEGVPSRFSGSGSGTDFTLTISSLQPEDVATYYCQHHLGTPY | 1F-ph-L |
| | TFGQGTKVEIK | VL |
| SEQ ID | DIQMTQSPSSLSASVGDRVTITCRASENQYGYLAWYQQKPGKVPKLL | BGA13- |
| NO: 132 | IYNTKNLVEGVPSRFSGSGSGTDFTLTISSLQPEDVATYYCQHHLGTPY | 1F-ph-M |
| | TFGQGTKVEIK | VL |

### Example 8. Further Engineering of Affinity Matured Humanized BGA13 Variants

Further engineering was made by introducing mutations in CDRs based on BGA131F-ph-M template, which included W33Y, Q54N and S59N in the VH and T51Y in the VL. This resulted in BGA1132A (W33Y (V4)), BGA132B (Q54N (VH)), BGA132C (S59N (VH)), BGA1131A (T51Y (VL)) which all had improved binding activities to BGA-1131F, with the most improved antibody finally resulting in the BGA113 antibody with the (W33Y (VH), T51Y (VL)) changes (Table 12), with the sequences shown in Table 13.

TABLE 12
Comparison of binding affinities to CHIM by SPR

| Sample ID | Ka(1/Ms) | Kd(1/s) | KD(M) | Rmax(RU) |
|---|---|---|---|---|
| BGA1311F-ph-M | 1.97E+04 | 2.48E−04 | 1.26E−08 | 97.2 |
| 1132A | 1.99E+04 | 2.82E−04 | 1.42E−08 | 129.1 |
| 1132B | 1.98E+04 | 3.80E−04 | 1.92E−08 | 71.1 |
| 1132C | 1.84E+04 | 3.94E−04 | 2.15E−08 | 59.4 |
| 1131A | 3.21E+04 | 4.63E−04 | 1.44E−08 | 92.9 |
| 113 | 3.15E+04 | 4.75E−04 | 1.51E−08 | 112.4 |

TABLE 13
Amino acid and nucleic acid sequences of BGA-113

| | | | |
|---|---|---|---|
| BGA113 | SEQ ID NO: | VH | QVQLVQSGAEVKKPGASVKVSCKASGYIF |
| | 133 | | TSYYLHWVRQAPGQGLEWIGYINPQTGY |
| | | | TSYAQKFQGRVTMTRDTSTSTVYMELSSL |
| | | | RSEDTAVYYCAREYGNYNYPLDYWGQGT |
| | | | LVTVSS |
| | SEQ ID NO: | VL | DIQMTQSPSSLSASVGDRVTITCRASENQY |
| | 15 | | GYLAWYQQKPGKVPKLLIYNYKNLVEGV |
| | | | PSRFSGSGSGTDFTLTISSLQPEDVATYYCQ |
| | | | HHLGTPYTFGQGTKVEIK |
| | SEQ ID NO: | VH DNA | CAGGTGCAGCTGGTGCAGAGCGGCGCG |
| | 134 | | GAAGTGAAAAAACCGGGCGCGAGCGTG |
| | | | AAAGTGAGCTGCAAAGCGAGCGGCTATA |
| | | | TTTTTACCAGCTATTACCTGCATTGGGTG |
| | | | CGCCAGGCGCCGGGCCAGGGCCTGGAAT |
| | | | GGATTGGCTATATTAACCCGCAGACCGGC |
| | | | TATACCAGCTATGCCCAGAAATTTCAGGG |
| | | | CCGCGTGACCATGACCCGCGATACCAGC |
| | | | ACCAGCACCGTGTATATGGAACTGAGCA |
| | | | GCCTGCGCAGCGAAGATACCGCGGTGTA |
| | | | TTATTGCGCGCGCGAATATGGCAACTATA |
| | | | ACTATCCGCTGGATTATTGGGGCCAGGGC |
| | | | ACCCTGGTGACCGTGAGCAGC |
| | SEQ ID NO: | VL DNA | GATATTCAGATGACCCAGAGCCCGAGCA |
| | 17 | | GCCTGAGCGCGAGCGTGGGCGATCGCGT |
| | | | GACCATTACCTGCCGCGCGAGCGAAAAC |
| | | | CAGTATGGCTATCTGGCGTGGTATCAGCA |
| | | | GAAACCGGGCAAAGTGCCGAAACTGCT |
| | | | GATTTATAACTATAAAAACCTGGTGGAAG |
| | | | GCGTGCCGAGCCGCTTTAGCGGCAGCGG |
| | | | CAGCGGCACCGATTTTACCCTGACCATTA |
| | | | GCAGCCTGCAGCCGGAAGATGTGGCGAC |
| | | | CTATTATTGCCAGCATCATCTGGGCACCC |
| | | | CGTATACCTTTGGCCAGGGCACCAAAGT |
| | | | GGAAATTAAA |

### Example 9. Optimization of BGA113

To further improve the biochemical/biophysical properties, optimization of BGA113 was made by introducing substitutions in CDR and framework regions (Table 14). The large, hydrophobic residues were chosen and changed to polar residues, except for K13 and Q53, which are selected based on observed differences among human VH germlines. The considerations include amino acid compositions, heat stability (Tm), surface hydrophobicity and isoelectronic points (pIs) while maintaining functional activities. The variants were expressed in Fab format by cloning into the vector pCANTAB-5E as described in Example 6. The Fab-containing supernatants were then screened by ELISA and SPR analysis for CEA binding. The variants without significant affinity reduction were selected and the residues which can tolerate substitutions were identified. It was demonstrated L92E in the light chain, K13E, Q54E, Y57D/E and Y57K in the heavy chain have minimal influence on the affinity. Thus, the BGA113 variants in IgG format with single identified mutation or combinations were expressed and purified as described in Example 8. SPR study and FACS analysis were performed and summarized in Table 14. It was confirmed that no changes in specificity and epitope occurred due to the introduced amino acid substitutions (data not shown). Taken together, the results demonstrated these single or combined mutations (K13E, Q54E, Y57D and Y57K in the heavy chain, L92E in the light chain) have minimal effects on the affinity, except for L92E, which slightly reduces the binding affinity to CEA. In summary, the Y57K change optimized the BGA113 antibody for expression, CEA binding and affinity, which resulted in BGA113K (Table 1).

TABLE 14
Summary of residues for substitution

| Residue | AA substitution |
|---|---|
| H: K13 | E |
| H: Y32 | H, N, Q, D, E, K |
| H: Y33 | H, N, Q, D, E, K |
| H: Q53 | A, D, G, N, S, T, Y, R, H, |
| H: Y57 | H, N, Q, D, E, K |
| H: Y100 | H, N, Q, D, E, K |
| H: Y105 | H, N, Q, D, E, K |
| L: V15 | T, P, L |
| L: Y30 | H, N, Q, D, E, K |
| L: Y32 | H, N, Q, D, E, K |
| L: Y49 | H, N, Q, D, E, K |
| L: P80 | S, T, A |
| L: L92 | H, N, Q, D, E, K |

TABLE 15
Summary of affinity measurement of BGA113 variants by SPR

| | | | | Affinity | |
|---|---|---|---|---|---|
| | | | | (MKN45 cells) | |
| BGA113 | Kinetics (huCEA) | | | | EC50 |
| variants | ka (1/Ms) | kd (1/s) | KD (M) | Top | (ug/ml) |
| L92E, Y57K | 3.34E+04 | 1.77E−04 | 5.29E−09 | 969.9 | 1.09 |
| L92E | 4.17E+04 | 1.88E−04 | 4.50E−09 | 945.4 | 1.19 |
| Q54E, Y57K | 1.10E+05 | 2.60E−04 | 2.36E−09 | 901.9 | 0.58 |
| Y57K | 1.02E+05 | 1.81E−04 | 1.77E−09 | 934.6 | 0.52 |
| K13E, Y57K | 1.28E+05 | 1.72E−04 | 1.34E−09 | 959.9 | 0.48 |
| Q54E, Y57E | 1.17E+05 | 2.50E−04 | 2.14E−09 | 926.9 | 0.65 |
| Y57D | 1.07E+05 | 1.95E−04 | 1.82E−09 | 949.3 | 0.56 |
| BGA113 | 1.13E+05 | 1.69E−04 | 1.49E−09 | 919.3 | 0.49 |

### Example 10. Binding Profiles of Anti-CEA Antibody BGA113K

BGA113K and a previously disclosed CEA antibody, designated as antibody 2F1 in U.S. 2012/0251529, were generated in human IgG1 format and characterized for their binding kinetics by SPR assays using BIAcore™ T-200 (GE Life Sciences).

To obtain this data, anti-human IgG (Fc) antibody was immobilized on an activated CM5 biosensor chip (Cat. No. BR100839, GE Life Sciences). The BGA113K antibody was flowed over the chip surface and captured by anti-human Fab antibody. Then a serial dilution (1.37 nM to 2150 nM) of soluble huCEA or cynoCEA(Cat.:CE5-C52H5, Acrobiosystem) were flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. BGA113K and and the 2F1 control antibody displayed different binding affinity. BGA113K has very high affinity for human CEA and also a comparable affinity for cynoCEA, as shown in Table 16 below.

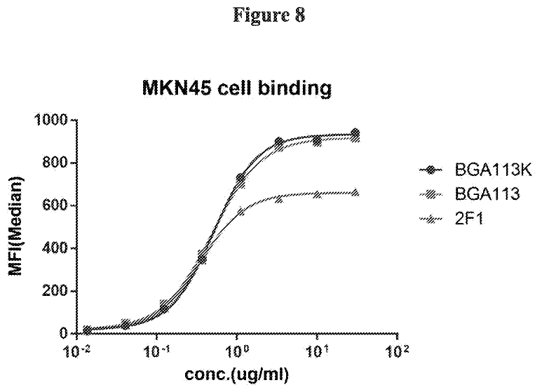

For flow cytometry, CEA-expressing MKN45 cells (10⁵ cells/well) were incubated with various concentrations of purified affinity-matured antibodies, followed by binding with Alexa Fluro-647-labeled anti-hu IgG Fc antibody (Cat. No. 409320, BioLegend, USA). Cell fluorescence was quantified using a flow cytometer (Guava easyCyte™ 8HT, Merck-Millipore, USA). As shown in FIG. 8, BGA-113K demonstrated specific binding to native CEA on living cells in a dose-responsive manner with EC50 of 2.92 ug/ml.

TABLE 16
Comparison of binding affinities of anti-CEA antibodies by SPR

| | BGA113K | | | 2F1 Antibody | | |
|---|---|---|---|---|---|---|
| | Ka (1/Ms) | Kd (1/s) | KD (M) | Ka (1/Ms) | Kd (1/s) | KD (M) |
| huCEA | 1.02E+05 | 1.81E−04 | 1.77E−09 | 4.50E+4 | 3.00E−3 | 6.50E−8 |
| cynoCEA | 2.67E+04 | 5.34E−04 | 1.99E−08 | ND | ND | ND |

### Example 11. Evaluation of Off-Target Specificity

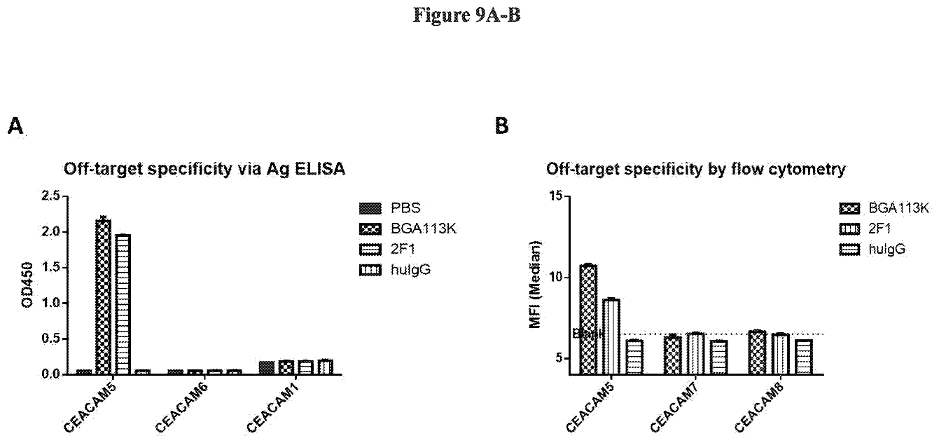

The off-target specificity of BGA113K was evaluated via ELISA and flow cytometry. For flow cytometry CEACAM3 (SEQ ID NO:101), CEACAM7 (SEQ ID NO:102) or CEACAM8 (SEQ ID NO:103) were transiently transfected into HEK293 cells (10⁵ cells/well) and then were incubated with 2 μg/ml purified BGA113K, followed by binding with Alexa Fluor-647-labeled anti-huIgG Fc antibody (Cat. No. 409320, BioLegend, USA). Cell fluorescence was quantified using a flow cytometer (Guava easyCyte™ 8HT, Merck-Millipore, USA). For antigen ELISA, CEACAM1(SEQ ID NO:100) (Cat. No. 10822-H08H, Sino Biological, China), CHIM (SEQ ID NO: 99), CEA (SEQ ID NO:91) or CEACAM6 (SEQ ID NO:93) were coated in 96-well plates at a concentration of 10 μg/ml overnight at 4° C. The HRP-linked anti-human Fc (Fc specific) IgG antibody (Cat. No. A0170, Sigma, USA) and substrate (Cat. No. 00-4201-56, eBioscience, USA) were used for development, and absorbance signal at the wavelength of 450 nm was measured using a plate reader (SpectraMax Paradigm, Molecular Devices, USA). As shown in FIGS. 9A and 9B, no cross-reactivity to other CEACAM family members were observed, thus BGA113K demonstrated specificity only for CEA (CEACAM5 in FIG. 9A-B).

### Example 12. Effects of Soluble huCEA on Binding of BGA113K to CEA Expressing Cells

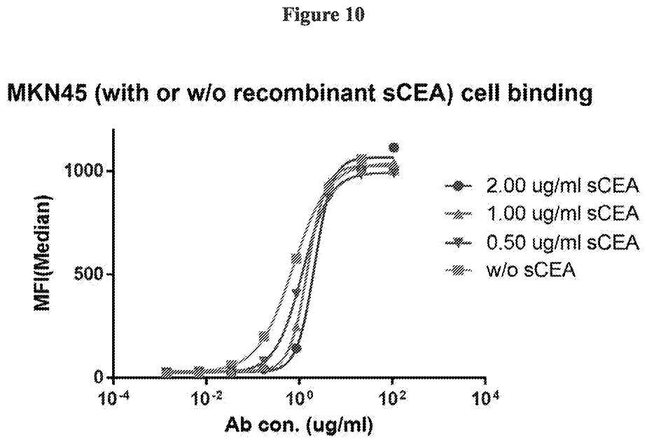

To determine if soluble CEA (sCEA) had any effect on the specific binding of BGA113K, various concentrations (0, 0.5, 1, 2 μg/ml) of recombinant soluble CEA was premixed with (0.01˜100 μg/ml) BGA113K and incubated for 5 min. The mixtures were then incubated with 2×10⁵ CEA expressing cells, such as MKN45 cells for 30 minutes at 4° C. The cells were stained with secondary antibody anti-huFc-APC (Cat. No. 409320, BioLegend, USA) and analyzed by flow cytometry. In the presence of 2 μg/ml of recombinant sCEA, the binding of BGA113K to CEA expressing cells was not affected. This result is shown for MKN45 cells (FIG. 10) and indicates BGA113K's specificity for the membrane bound form of CEA.

### Example 13. BGA113 Induces Potent ADCC Effects on CEA⁺ Tumor Cells

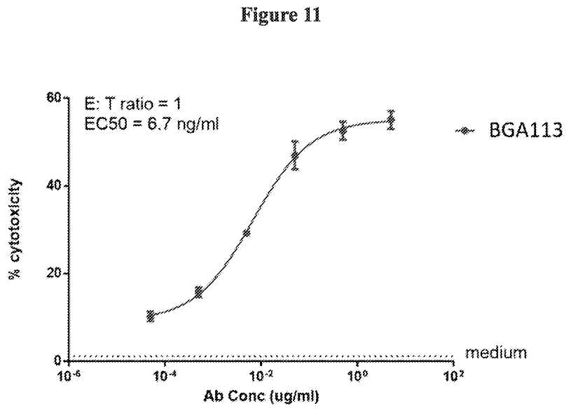

To determine whether BGA113 in the wild-type IgG1 format can induce antibody dependent cytotoxicity (ADCC), CD16(V158)-expressing NK92MI cells (NK92MI/CD16V) were used as effector cells and were co-cultured with mouse colon cancer cells (CT26-ATCC CRL-2638) expressing CEA. The co-culture was performed at an E:T ratio of 1:1 for 5 hours in the presence of BGA113 at indicated concentrations (0.00005-5 μg/ml), and cytotoxicity was determined by Lactate dehydrogenase (LDH) release. The amounts of LDH in the supernatant were measured using the CytoTox™ 96 Non-Radioactive Cytotoxicity Assay kit (Promega, Madison, WI), and the amount of specific lysis was calculated according to the manufacturer's instruction. As shown in FIG. 11, BGA113 could induce ADCC in vitro with an EC₅₀ around 6.7 ng/ml.

### Example 14. In Vivo Anti-Tumor Efficacy of BGA113

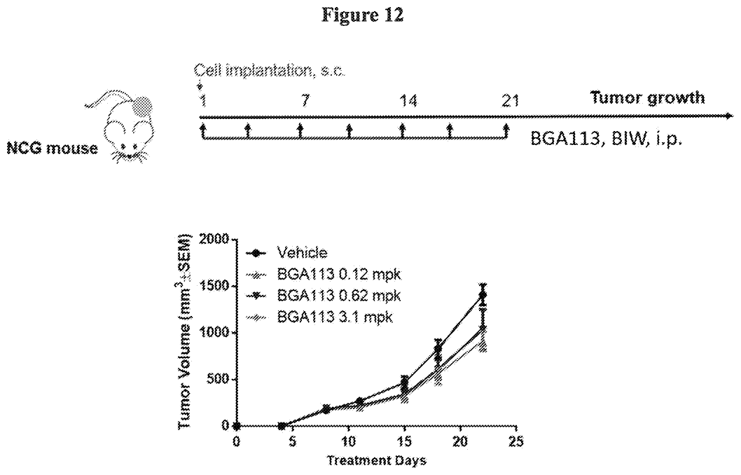

To determine the in vivo efficacy of BGA113 against CEA⁺ tumor cells, NK92MI/CD16V cells (5×10⁶) were mixed with CT26/CEA cells (10⁶) and injected subcutaneously into NCG mice. BGA113 (0.12, 0.62 or 3.1 mg/kg) or vehicle control was given twice a week starting on the day of tumor injection (7 mice per group). As compared to vehicle, BGA113 at 3.1 mg/kg dosage showed a low amount of tumor inhibition, although the difference from vehicle control was not statistically significant (P>0.05) (FIG. 12).

### Example 15. Generation of Recombinant Proteins and Stable Cell Lines

### CD137 Recombinant Proteins for Phage Campaign and Binding Assays

To discover VH domain antibodies against CD137 with cross-binding of human and Macaca mulatta CD137, but without off-target binding with other human TNF receptor members, several recombinant proteins were designed and expressed for phage panning and screening (see Table 17). The cDNA coding regions for the full-length human CD137 (SEQ ID NO: 135) was ordered based on the CD137 GenBank sequence (Accession No: NM_001561.4, the gene is available from Sinobio, Cat.: HG10041-M). Human CD137 ligand (TNFSF9) (SEQ ID NO:145) was ordered based on (Accession No: NM_003811.3, the gene is available from Sinobio, Cat.: HG15693-G). Monkey (Macaca mulatta) CD137 (SEQ ID NO:151) was ordered based on (Accession No: NM_001266128.1, the gene is available from Genscript, Cat.: OMb00270). The full-length human CD40 (SEQ ID NO: 157) was ordered based on (Accession No: NM_001250.4, the gene is available from Sinobio, Cat.: HG10774-M). OX40 (SEQ ID NO: 163) was ordered based on (Accession No: NM_003327.2, the gene is available from Sinobio, Cat.: HG10481-UT). In brief, the coding region of extracellular domain (ECD) consisting of amino acid (AA) 24-183 of huCD137 (SEQ ID NO: 137), the coding region of ECD consisting of AA 71-254 of human CD137 ligand (SEQ ID NO: 147), the coding region of ECD consisting of AA 24-186 of cynoCD137 (SEQ ID NO: 153), and the coding region of ECD consisting of AA 1-194 of human CD40 (SEQ ID NO: 159) were PCR-amplified, respectively. The coding region of mIgG2a Fc (SEQ ID NO: 143) was PCR-amplified, and then conjugated with ECDs of human CD137, human CD137 ligand, monkey CD137 or human CD40 by overlap-PCR to make mIgG2a Fc-fusion proteins. PCR products were then cloned into a pcDNA3.1-based expression vector (Invitrogen, Carlsbad, CA, USA), which resulted in five recombinant mIgG2a Fc-fusion protein expression plasmids, human CD137 ECD-mIgG2a, human CD137 ligand-mIgG2a, cyno CD137 ECD-mIgG2a and human CD40 ECD-mIgG2a. Alternatively the coding regions of ECD consisting of AA 24-183 (SEQ ID NO: 137) of huCD137 (SEQ ID NO: 135) and the coding region of ECD consisting of AA 1-216 of human OX40 (SEQ ID NO: 165) were also cloned into a pcDNA3.1-based expression vector (Invitrogen, Carlsbad, CA, USA) with C-terminus fused with 6×His tags, which resulted in human CD137-his and human OX40-his, respectively. For the recombinant fusion protein production, plasmids were transiently transfected into a HEK293-based mammalian cell expression system (developed in house) and cultured for 5-7 days in a CO₂ incubator equipped with rotating shaker. The supernatants containing the recombinant proteins were collected and cleared by centrifugation. Recombinant proteins were purified using a Protein A column (Cat.: 17127901, GE Life Sciences) or a Ni-NTA agarose (Cat.: R90115, Invitrogen). All recombinant proteins were dialyzed against phosphate buffered saline (PBS) and stored in −80° C. freezer in small aliquots.

### Stable Expression Cell Lines

To establish stable cell lines that express full-length human CD137 (huCD137), huCD137 sequences were cloned into a retroviral vector pFB-Neo (Cat.: 217561, Agilent, USA). Dual-tropic retroviral vectors were generated according to a previous protocol (Zhang, et al., (2005) Blood, 106, 1544-1551.). Vectors containing huCD137 were transduced into Hut78 cells (ATCC, TIB-161) or NK92-mi cells (ATCC, CRL-2408), to generate the huCD137 expressing cell lines, Hut78/huCD137 or NK92-mi/huCD137. huCD137 expressing cell lines were selected by culture in medium containing 1000 FBS with G418, and then verified via FACS.

TABLE 17
Sequences for recombinant CD137 proteins

| CONSTRUCT | SEQ ID NO: | SEQUENCE |
|---|---|---|
| human CD137 FL | SEQ ID NO: | MGNSCYNIVATLLLVLNFERTRSLQDPCSNC |
| AA | 135 | PAGTFCDNNRNQICSPCPPNSFSSAGGQRTC |
| | | DICRQCKGVFRTRKECSSTSNAECDCTPGFH |
| | | CLGAGCSMCEQDCKQGQELTKKGCKDCCF |
| | | GTFNDQKRGICRPWTNCSLDGKSVLVNGTK |
| | | ERDVVCGPSPADLSPGASSVTPPAPAREPGHS |
| | | PQIISFFLALTSTALLFLLFFLTLRFSVVKRGR |
| | | KKLLYIFKQPFMRPVQTTQEEDGCSCRFPEE |
| | | EEGGCEL |
| human CD137 FL | SEQ ID NO: | ATGGGAAACAGCTGTTACAACATAGTAGCC |
| DNA | 136 | ACTCTGTTGCTGGTCCTCAACTTTGAGAGG |
| | | ACAAGATCATTGCAGGATCCTTGTAGTAAC |
| | | TGCCCAGCTGGTACATTCTGTGATAATAAC |
| | | AGGAATCAGATTTGCAGTCCCTGTCCTCCA |
| | | AATAGTTTCTCCAGCGCAGGTGGACAAAG |
| | | GACCTGTGACATATGCAGGCAGTGTAAAG |
| | | GTGTTTTCAGGACCAGGAAGGAGTGTTCC |
| | | TCCACCAGCAATGCAGAGTGTGACTGCAC |
| | | TCCAGGGTTTCACTGCCTGGGGGCAGGAT |
| | | GCAGCATGTGTGAACAGGATTGTAAACAA |
| | | GGTCAAGAACTGACAAAAAAAGGTTGTAA |
| | | AGACTGTTGCTTTGGGACATTTAACGATCA |
| | | GAAACGTGGCATCTGTCGACCCTGGACAA |
| | | ACTGTTCTTTGGATGGAAAGTCTGTGCTTG |
| | | TGAATGGGACGAAGGAGAGGGACGTGGTC |
| | | TGTGGACCATCTCCAGCCGACCTCTCTCCG |
| | | GGAGCATCCTCTGTGACCCCGCCTGCCCCT |
| | | GCGAGAGAGCCAGGACACTCTCCGCAGAT |
| | | CATCTCCTTCTTTCTTGCGCTGACGTCGAC |
| | | TGCGTTGCTCTTCCTGCTGTTCTTCCTCAC |
| | | GCTCCGTTTCTCTGTTGTTAAACGGGGCAG |
| | | AAAGAAACTCCTGTATATATTCAAACAACC |
| | | ATTTATGAGACCAGTACAAACTACTCAAGA |
| | | GGAAGATGGCTGTAGCTGCCGATTTCCAGA |
| | | AGAAGAAGAAGGAGGATGTGAACTG |
| human CD137 AA | SEQ ID NO: | LQDPCSNCPAGTFCDNNRNQICSPCPPNSFSS |
| 24-183 | 137 | AGGQRTCDICRQCKGVFRTRKECSSTSNAEC |
| | | DCTPGFHCLGAGCSMCEQDCKQGQELTKK |
| | | GCKDCCFGTFNDQKRGICRPWTNCSLDGKS |
| | | VLVNGTKERDVVCGPSPADLSPGASSVTPPA |
| | | PAREPGH |
| human CD137 24- | SEQ ID NO: | CTGCAGGATCCTTGTAGTAACTGCCCAGCT |
| 183 DNA | 138 | GGTACATTCTGTGATAATAACAGGAATCAG |
| | | ATTTGCAGTCCCTGTCCTCCAAATAGTTTCT |
| | | CCAGCGCAGGTGGACAAAGGACCTGTGAC |
| | | ATATGCAGGCAGTGTAAAGGTGTTTTCAGG |
| | | ACCAGGAAGGAGTGTTCCTCCACCAGCAA |
| | | TGCAGAGTGTGACTGCACTCCAGGGTTTC |
| | | ACTGCCTGGGGGCAGGATGCAGCATGTGT |
| | | GAACAGGATTGTAAACAAGGTCAAGAACT |
| | | GACAAAAAAAGGTTGTAAAGACTGTTGCT |
| | | TTGGGACATTTAACGATCAGAAACGTGGC |
| | | ATCTGTCGACCCTGGACAAACTGTTCTTTG |
| | | GATGGAAAGTCTGTGCTTGTGAATGGGAC |
| | | GAAGGAGAGGGACGTGGTCTGTGGACCAT |
| | | CTCCAGCCGACCTCTCTCCGGGAGCATCCT |
| | | CTGTGACCCCGCCTGCCCCTGCGAGAGAG |
| | | CCAGGACAC |
| human CD137-his | SEQ ID NO: | LQDPCSNCPAGTFCDNNRNQICSPCPPNSFSS |
| | 139 | AGGQRTCDICRQCKGVFRTRKECSSTSNAEC |
| | | DCTPGFHCLGAGCSMCEQDCKQGQELTKK |
| | | GCKDCCFGTFNDQKRGICRPWTNCSLDGKS |
| | | VLVNGTKERDVVCGPSPADLSPGASSVTPPA |
| | | PAREPGHHHHHHHGGGLNDIFEAQKIEWHE |
| human CD137-his | SEQ ID NO: | CTGCAGGATCCTTGTAGTAACTGCCCAGCT |
| DNA | 140 | GGTACATTCTGTGATAATAACAGGAATCAG |
| | | ATTTGCAGTCCCTGTCCTCCAAATAGTTTCT |
| | | CCAGCGCAGGTGGACAAAGGACCTGTGAC |
| | | ATATGCAGGCAGTGTAAAGGTGTTTTCAGG |
| | | ACCAGGAAGGAGTGTTCCTCCACCAGCAA |
| | | TGCAGAGTGTGACTGCACTCCAGGGTTTC |
| | | ACTGCCTGGGGGCAGGATGCAGCATGTGT |
| | | GAACAGGATTGTAAACAAGGTCAAGAACT |
| | | GACAAAAAAAGGTTGTAAAGACTGTTGCT |
| | | TTGGGACATTTAACGATCAGAAACGTGGC |
| | | ATCTGTCGACCCTGGACAAACTGTTCTTTG |
| | | GATGGAAAGTCTGTGCTTGTGAATGGGAC |
| | | GAAGGAGAGGGACGTGGTCTGTGGACCAT |
| | | CTCCAGCCGACCTCTCTCCGGGAGCATCCT |
| | | CTGTGACCCCGCCTGCCCCTGCGAGAGAG |
| | | CCAGGACACCATCACCATCACCATCACGGA |
| | | GGCGGTCTGAACGACATCTTCGAGGCTCA |
| | | GAAAATCGAATGGCACGAA |
| human CD137 | SEQ ID NO: | LQDPCSNCPAGTFCDNNRNQICSPCPPNSFSS |
| ECD-mIgG2a AA | 141 | AGGQRTCDICRQCKGVFRTRKECSSTSNAEC |
| | | DCTPGFHCLGAGCSMCEQDCKQGQELTKK |
| | | GCKDCCFGTFNDQKRGICRPWTNCSLDGKS |
| | | VLVNGTKERDVVCGPSPADLSPGASSVTPPA |
| | | PAREPGHEPRGPTIKPCPPCKCPAPNLLGGPS |
| | | VFIFPPKIKDVLMISLSPIVTCVVVDVSEDDP |
| | | DVQISWFVNNVEVHTAQTQTHREDYNSTLR |
| | | VVSALPIQHQDWMSGKEFKCKVNNKDLPAP |
| | | IERTISKPKGSVRAPQVYVLPPPEEEMTKKQ |
| | | VTLTCMVTDFMPEDIYVEWTNNGKTELNYK |
| | | NTEPVLDSDGSYFMYSKLRVEKKNWVERNS |
| | | YSCSVVHEGLHNHHTTKSFSRTPGK |
| human CD137 | SEQ ID NO: | CTGCAGGATCCTTGTAGTAACTGCCCAGCT |
| ECD-mIgG2a | 142 | GGTACATTCTGTGATAATAACAGGAATCAG |
| DNA | | ATTTGCAGTCCCTGTCCTCCAAATAGTTTCT |
| | | CCAGCGCAGGTGGACAAAGGACCTGTGAC |
| | | ATATGCAGGCAGTGTAAAGGTGTTTTCAGG |
| | | ACCAGGAAGGAGTGTTCCTCCACCAGCAA |
| | | TGCAGAGTGTGACTGCACTCCAGGGTTTC |
| | | ACTGCCTGGGGGCAGGATGCAGCATGTGT |
| | | GAACAGGATTGTAAACAAGGTCAAGAACT |
| | | GACAAAAAAAGGTTGTAAAGACTGTTGCT |
| | | TTGGGACATTTAACGATCAGAAACGTGGC |
| | | ATCTGTCGACCCTGGACAAACTGTTCTTTG |
| | | GATGGAAAGTCTGTGCTTGTGAATGGGAC |
| | | GAAGGAGAGGGACGTGGTCTGTGGACCAT |
| | | CTCCAGCCGACCTCTCTCCGGGAGCATCCT |
| | | CTGTGACCCCGCCTGCCCCTGCGAGAGAG |
| | | CCAGGACACGAGCCCAGAGGGCCCACAAT |
| | | CAAGCCCTGTCCTCCATGCAAATGCCCAGC |
| | | ACCTAACCTCTTGGGTGGACCATCCGTCTT |
| | | CATCTTCCCTCCAAAGATCAAGGATGTACT |
| | | CATGATCTCCCTGAGCCCCATAGTCACATG |
| | | TGTGGTGGTGGATGTGAGCGAGGATGACC |
| | | CAGATGTCCAGATCAGCTGGTTTGTGAACA |
| | | ACGTGGAAGTACACACAGCTCAGACACAA |
| | | ACCCATAGAGAGGATTACAACAGTACTCTC |
| | | CGGGTGGTCAGTGCCCTCCCCATCCAGCAC |
| | | CAGGACTGGATGAGTGGCAAGGAGTTCAA |
| | | ATGCAAGGTCAACAACAAAGACCTCCCAG |
| | | CGCCCATCGAGAGAACCATCTCAAAACCC |
| | | AAAGGGTCAGTAAGAGCTCCACAGGTATAT |
| | | GTCTTGCCTCCACCAGAAGAAGAGATGAC |
| | | TAAGAAACAGGTCACTCTGACCTGCATGG |
| | | TCACAGACTTCATGCCTGAAGACATTTACG |
| | | TGGAGTGGACCAACAACGGGAAAACAGA |
| | | GCTAAACTACAAGAACACTGAACCAGTCC |
| | | TGGACTCTGATGGTTCTTACTTCATGTACA |
| | | GCAAGCTGAGAGTGGAAAAGAAGAACTG |
| | | GGTGGAAAGAAATAGCTACTCCTGTTCAGT |
| | | GGTCCACGAGGGTCTGCACAATCACCACA |
| | | CGACTAAGAGCTTCTCCCGGACTCCGGGTA |
| | | AA |
| mIgG2a AA | SEQ ID NO: | EPRGPTIKPCPPCKCPAPNLLGGPSVFIFPPKI |
| | 143 | KDVLMISLSPIVTCVVVDVSEDDPDVQISWF |
| | | VNNVEVHTAQTQTHREDYNSTLRVVSALPI |
| | | QHQDWMSGKEFKCKVNNKDLPAPIERTISK |
| | | PKGSVRAPQVYVLPPPEEEMTKKQVTLTCM |
| | | VTDFMPEDIYVEWTNNGKTELNYKNTEPVL |
| | | DSDGSYFMYSKLRVEKKNWVERNSYSCSV |
| | | VHEGLHNHHTTKSFSRTPGK |
| mIgG2a DNA | SEQ ID NO: | GAGCCCAGAGGGCCCACAATCAAGCCCTG |
| | 144 | TCCTCCATGCAAATGCCCAGCACCTAACCT |
| | | CTTGGGTGGACCATCCGTCTTCATCTTCCC |
| | | TCCAAAGATCAAGGATGTACTCATGATCTC |
| | | CCTGAGCCCCATAGTCACATGTGTGGTGGT |
| | | GGATGTGAGCGAGGATGACCCAGATGTCC |
| | | AGATCAGCTGGTTTGTGAACAACGTGGAA |
| | | GTACACACAGCTCAGACACAAACCCATAG |
| | | AGAGGATTACAACAGTACTCTCCGGGTGGT |
| | | CAGTGCCCTCCCCATCCAGCACCAGGACT |
| | | GGATGAGTGGCAAGGAGTTCAAATGCAAG |
| | | GTCAACAACAAAGACCTCCCAGCGCCCAT |
| | | CGAGAGAACCATCTCAAAACCCAAAGGGT |
| | | CAGTAAGAGCTCCACAGGTATATGTCTTGC |
| | | CTCCACCAGAAGAAGAGATGACTAAGAAA |
| | | CAGGTCACTCTGACCTGCATGGTCACAGA |
| | | CTTCATGCCTGAAGACATTTACGTGGAGTG |
| | | GACCAACAACGGGAAAACAGAGCTAAACT |
| | | ACAAGAACACTGAACCAGTCCTGGACTCT |
| | | GATGGTTCTTACTTCATGTACAGCAAGCTG |
| | | AGAGTGGAAAAGAAGAACTGGGTGGAAA |
| | | GAAATAGCTACTCCTGTTCAGTGGTCCACG |
| | | AGGGTCTGCACAATCACCACACGACTAAG |
| | | AGCTTCTCCCGGACTCCGGGTAAA |
| human CD137 | SEQ ID NO: | MEYASDASLDPEAPWPPAPRARACRVLPWA |
| ligand AA | 145 | LVAGLLLLLLLAAACAVFLACPWAVSGARAS |
| | | PGSAASPRLREGPELSPDDPAGLLDLRQGMF |
| | | AQLVAQNVLLIDGPLSWYSDPGLAGVSLTG |
| | | GLSYKEDTKELVVAKAGVYYVFFQLELRRV |
| | | VAGEGSGSVSLALHLQPLRSAAGAAALALT |
| | | VDLPPASSEARNSAFGFQGRLLHLSAGQRLG |
| | | VHLHTEARARHAWQLTQGATVLGLFRVTPEI |
| | | PAGLPSPRSE |
| human CD137 | SEQ ID NO: | ATGGAATACGCCTCTGACGCTTCACTGGAC |
| ligand cDNA | 146 | CCCGAAGCCCCGTGGCCTCCCGCGCCCCG |
| | | CGCTCGCGCCTGCCGCGTACTGCCTTGGGC |
| | | CCTGGTCGCGGGGCTGCTGCTGCTGCTGCT |
| | | GCTCGCTGCCGCCTGCGCCGTCTTCCTCGC |
| | | CTGCCCCTGGGCCGTGTCCGGGGCTCGCG |
| | | CCTCGCCCGGCTCCGCGGCCAGCCCGAGA |
| | | CTCCGCGAGGGTCCCGAGCTTTCGCCCGA |
| | | CGATCCCGCCGGCCTCTTGGACCTGCGGCA |
| | | GGGCATGTTTGCGCAGCTGGTGGCCCAAA |
| | | ATGTTCTGCTGATCGATGGGCCCCTGAGCT |
| | | GGTACAGTGACCCAGGCCTGGCAGGCGTG |
| | | TCCCTGACGGGGGGCCTGAGCTACAAAGA |
| | | GGACACGAAGGAGCTGGTGGTGGCCAAG |
| | | GCTGGAGTCTACTATGTCTTCTTTCAACTA |
| | | GAGCTGCGGCGCGTGGTGGCCGGCGAGGG |
| | | CTCAGGCTCCGTTTCACTTGCGCTGCACCT |
| | | GCAGCCACTGCGCTCTGCTGCTGGGGCCG |
| | | CCGCCCTGGCTTTGACCGTGGACCTGCCAC |
| | | CCGCCTCCTCCGAGGCTCGGAACTCGGCC |
| | | TTCGGTTTCCAGGGCCGCTTGCTGCACCTG |
| | | AGTGCCGGCCAGCGCCTGGGCGTCCATCTT |
| | | CACACTGAGGCCAGGGCACGCCATGCCTG |
| | | GCAGCTTACCCAGGGCGCCACAGTCTTGG |
| | | GACTCTTCCGGGTGACCCCCGAAATCCCA |
| | | GCCGGACTCCCTTCACCGAGGTCGGAATA |
| | | A |
| human CD137 | SEQ ID NO: | REGPELSPDDPAGLLDLRQGMFAQLVAQNV |
| ligand 71-254 AA | 147 | LLIDGPLSWYSDPGLAGVSLTGGLSYKEDTK |
| | | ELVVAKAGVYYVFFQLELRRVVAGEGSGSV |
| | | SLALHLQPLRSAAGAAALALTVDLPPASSEA |
| | | RNSAFGFQGRLLHLSAGQRLGVHLHTEARA |
| | | RHAWQLTQGATVLGLFRVTPEIPAGLPSPRSE |
| human CD137 | SEQ ID NO: | CGCGAGGGTCCCGAGCTTTCGCCCGACGA |
| ligand 71-254 DNA | 148 | TCCCGCCGGCCTCTTGGACCTGCGGCAGG |
| | | GCATGTTTGCGCAGCTGGTGGCCCAAAATG |
| | | TTCTGCTGATCGATGGGCCCCTGAGCTGGT |
| | | ACAGTGACCCAGGCCTGGCAGGCGTGTCC |
| | | CTGACGGGGGGCCTGAGCTACAAAGAGGA |
| | | CACGAAGGAGCTGGTGGTGGCCAAGGCTG |
| | | GAGTCTACTATGTCTTCTTTCAACTAGAGC |
| | | TGCGGCGCGTGGTGGCCGGCGAGGGCTCA |
| | | GGCTCCGTTTCACTTGCGCTGCACCTGCAG |
| | | CCACTGCGCTCTGCTGCTGGGGCCGCCGC |
| | | CCTGGCTTTGACCGTGGACCTGCCACCCGC |
| | | CTCCTCCGAGGCTCGGAACTCGGCCTTCG |
| | | GTTTCCAGGGCCGCTTGCTGCACCTGAGTG |
| | | CCGGCCAGCGCCTGGGCGTCCATCTTCACA |
| | | CTGAGGCCAGGGCACGCCATGCCTGGCAG |
| | | CTTACCCAGGGCGCCACAGTCTTGGGACT |
| | | CTTCCGGGTGACCCCCGAAATCCCAGCCG |
| | | GACTCCCTTCACCGAGGTCGGAA |
| human CD137 | SEQ ID NO: | EPRGPTIKPCPPCKCPAPNLLGGPSVFIFPPKI |
| ligand-mIgG2a AA | 149 | KDVLMISLSPIVTCVVVDVSEDDPDVQISWF |
| | | VNNVEVHTAQTQTHREDYNSTLRVVSALPI |
| | | QHQDWMSGKEFKCKVNNKDLPAPIERTISK |
| | | PKGSVRAPQVYVLPPPEEEMTKKQVTLTCM |
| | | VTDFMPEDIYVEWTNNGKTELNYKNTEPVL |
| | | DSDGSYFMYSKLRVEKKNWVERNSYSCSV |
| | | VHEGLHNHHTTKSFSRTPGKGGGGSREGPE |
| | | LSPDDPAGLLDLRQGMFAQLVAQNVLLIDGP |
| | | LSWYSDPGLAGVSLTGGLSYKEDTKELVVA |
| | | KAGVYYVFFQLELRRVVAGEGSGSVSLALH |
| | | LQPLRSAAGAAALALTVDLPPASSEARNSAF |
| | | GFQGRLLHLSAGQRLGVHLHTEARARHAW |
| | | QLTQGATVLGLFRVTPEIPAGLPSPRSE |
| human CD137 | SEQ ID NO: | GAGCCCAGAGGGCCCACAATCAAGCCCTG |
| ligand-mIgG2a | 150 | TCCTCCATGCAAATGCCCAGCACCTAACCT |
| DNA | | CTTGGGTGGACCATCCGTCTTCATCTTCCC |
| | | TCCAAAGATCAAGGATGTACTCATGATCTC |
| | | CCTGAGCCCCATAGTCACATGTGTGGTGGT |
| | | GGATGTGAGCGAGGATGACCCAGATGTCC |
| | | AGATCAGCTGGTTTGTGAACAACGTGGAA |
| | | GTACACACAGCTCAGACACAAACCCATAG |
| | | AGAGGATTACAACAGTACTCTCCGGGTGGT |
| | | CAGTGCCCTCCCCATCCAGCACCAGGACT |
| | | GGATGAGTGGCAAGGAGTTCAAATGCAAG |
| | | GTCAACAACAAAGACCTCCCAGCGCCCAT |
| | | CGAGAGAACCATCTCAAAACCCAAAGGGT |
| | | CAGTAAGAGCTCCACAGGTATATGTCTTGC |
| | | CTCCACCAGAAGAAGAGATGACTAAGAAA |
| | | CAGGTCACTCTGACCTGCATGGTCACAGA |
| | | CTTCATGCCTGAAGACATTTACGTGGAGTG |
| | | GACCAACAACGGGAAAACAGAGCTAAACT |
| | | ACAAGAACACTGAACCAGTCCTGGACTCT |
| | | GATGGTTCTTACTTCATGTACAGCAAGCTG |
| | | AGAGTGGAAAAGAAGAACTGGGTGGAAA |
| | | GAAATAGCTACTCCTGTTCAGTGGTCCACG |
| | | AGGGTCTGCACAATCACCACACGACTAAG |
| | | AGCTTCTCCCGGACTCCGGGTAAAGGTGG |
| | | AGGCGGTTCACGCGAGGGTCCCGAGCTTT |
| | | CGCCCGACGATCCCGCCGGCCTCTTGGACC |
| | | TGCGGCAGGGCATGTTTGCGCAGCTGGTG |
| | | GCCCAAAATGTTCTGCTGATCGATGGGCCC |
| | | CTGAGCTGGTACAGTGACCCAGGCCTGGC |
| | | AGGCGTGTCCCTGACGGGGGGCCTGAGCT |
| | | ACAAAGAGGACACGAAGGAGCTGGTGGT |
| | | GGCCAAGGCTGGAGTCTACTATGTCTTCTT |
| | | TCAACTAGAGCTGCGGCGCGTGGTGGCCG |
| | | GCGAGGGCTCAGGCTCCGTTTCACTTGCG |
| | | CTGCACCTGCAGCCACTGCGCTCTGCTGCT |
| | | GGGGCCGCCGCCCTGGCTTTGACCGTGGA |
| | | CCTGCCACCCGCCTCCTCCGAGGCTCGGA |
| | | ACTCGGCCTTCGGTTTCCAGGGCCGCTTGC |
| | | TGCACCTGAGTGCCGGCCAGCGCCTGGGC |
| | | GTCCATCTTCACACTGAGGCCAGGGCACG |
| | | CCATGCCTGGCAGCTTACCCAGGGCGCCA |
| | | CAGTCTTGGGACTCTTCCGGGTGACCCCCG |
| | | AAATCCCAGCCGGACTCCCTTCACCGAGG |
| | | TCGGAA |
| cyno CD137 FL | SEQ ID NO: | MGNSCYNIVATLLLVLNFERTRSLQDLCSNC |
| AA | 151 | PAGTFCDNNRSQICSPCPPNSFSSAGGQRTCD |
| | | ICRQCKGVFKTRKECSSTSNAECDCISGYHC |
| | | LGAECSMCEQDCKQGQELTKKGCKDCCFG |
| | | TFNDQKRGICRPWTNCSLDGKSVLVNGTKE |
| | | RDVVCGPSPADLSPGASSATPPAPAREPGHSP |
| | | QIIFFLALTSTVVLFLLFFLVLRFSVVKRSRKK |
| | | LLYIFKQPFMRPVQTTQEEDGCSCRFPEEEE |
| | | GGCEL |
| cyno CD137 FL | SEQ ID NO: | ATGGGAAACAGCTGTTACAACATAGTAGCC |
| DNA | 152 | ACTCTGTTGCTGGTCCTGAACTTTGAGAGG |
| | | ACAAGATCCTTGCAGGATCTGTGTAGTAAC |
| | | TGCCCAGCTGGTACATTCTGTGATAATAAC |
| | | AGGAGTCAGATTTGCAGTCCCTGTCCTCCA |
| | | AATAGTTTCTCCAGCGCAGGTGGACAAAG |
| | | GACCTGTGACATATGCAGGCAGTGTAAAG |
| | | GTGTTTTCAAGACCAGGAAGGAGTGTTCC |
| | | TCCACCAGCAATGCAGAGTGTGACTGCATT |
| | | TCAGGATATCACTGCCTGGGGGCAGAATGC |
| | | AGCATGTGTGAACAGGATTGTAAACAAGG |
| | | TCAAGAATTGACAAAAAAAGGTTGTAAAG |
| | | ACTGTTGCTTTGGGACATTTAATGACCAGA |
| | | AACGTGGCATCTGTCGACCCTGGACAAAC |
| | | TGTTCTTTGGATGGAAAGTCTGTGCTTGTG |
| | | AATGGGACGAAGGAGAGGGACGTGGTCTG |
| | | CGGACCATCTCCAGCCGACCTCTCTCCAGG |
| | | AGCATCCTCTGCGACCCCGCCTGCCCCTGC |
| | | GAGAGAGCCAGGACACTCTCCGCAGATCA |
| | | TCTTCTTTCTTGCGCTGACTTCGACTGTGG |
| | | TACTCTTCCTGCTGTTCTTCCTCGTGCTCCG |
| | | TTTCTCTGTTGTTAAACGGAGCAGAAAGA |
| | | AACTCCTGTATATATTCAAACAACCATTTAT |
| | | GAGACCGGTACAAACCACTCAAGAGGAAG |
| | | ATGGATGTAGCTGCCGATTTCCAGAAGAAG |
| | | AAGAAGGAGGATGTGAACTGTGA |
| cyno CD137 ECD | SEQ ID NO: | LQDLCSNCPAGTFCDNNRSQICSPCPPNSFSS |
| 24-186 AA | 153 | AGGQRTCDICRQCKGVFKTRKECSSTSNAEC |
| | | DCISGYHCLGAECSMCEQDCKQGQELTKKG |
| | | CKDCCFGTFNDQKRGICRPWTNCSLDGKSV |
| | | LVNGTKERDVVCGPSPADLSPGASSATPPAP |
| | | AREPGHSPQ |
| cyno CD137 ECD | SEQ ID NO: | CTGCAGGATCTGTGTAGTAACTGCCCAGCT |
| 24-186 DNA | 154 | GGTACATTCTGTGATAATAACAGGAGTCAG |
| | | ATTTGCAGTCCCTGTCCTCCAAATAGTTTCT |
| | | CCAGCGCAGGTGGACAAAGGACCTGTGAC |
| | | ATATGCAGGCAGTGTAAAGGTGTTTTCAAG |
| | | ACCAGGAAGGAGTGTTCCTCCACCAGCAA |
| | | TGCAGAGTGTGACTGCATTTCAGGATATCA |
| | | CTGCCTGGGGGCAGAATGCAGCATGTGTG |
| | | AACAGGATTGTAAACAAGGTCAAGAATTG |
| | | ACAAAAAAAGGTTGTAAAGACTGTTGCTT |
| | | TGGGACATTTAATGACCAGAAACGTGGCAT |
| | | CTGTCGACCCTGGACAAACTGTTCTTTGGA |
| | | TGGAAAGTCTGTGCTTGTGAATGGGACGA |
| | | AGGAGAGGGACGTGGTCTGCGGACCATCT |
| | | CCAGCCGACCTCTCTCCAGGAGCATCCTCT |
| | | GCGACCCCGCCTGCCCCTGCGAGAGAGCC |
| | | AGGACACTCTCCGCAG |
| cyno CD137 ECD- | SEQ ID NO: | LQDLCSNCPAGTFCDNNRSQICSPCPPNSFSS |
| mIgG2a AA | 155 | AGGQRTCDICRQCKGVFKTRKECSSTSNAEC |
| | | DCISGYHCLGAECSMCEQDCKQGQELTKKG |
| | | CKDCCFGTFNDQKRGICRPWTNCSLDGKSV |
| | | LVNGTKERDVVCGPSPADLSPGASSATPPAP |
| | | AREPGHSPQEPRGPTIKPCPPCKCPAPNLLGG |
| | | PSVFIFPPKIKDVLMISLSPIVTCVVVDVSEDD |
| | | PDVQISWFVNNVEVHTAQTQTHREDYNSTL |
| | | RVVSALPIQHQDWMSGKEFKCKVNNKDLPA |
| | | PIERTISKPKGSVRAPQVYVLPPPEEEMTKKQ |
| | | VTLTCMVTDFMPEDIYVEWTNNGKTELNYK |
| | | NTEPVLDSDGSYFMYSKLRVEKKNWVERNS |
| | | YSCSVVHEGLHNHHTTKSFSRTPGK |
| cyno CD137 ECD- | SEQ ID NO: | CTGCAGGATCTGTGTAGTAACTGCCCAGCT |
| mIgG2a DNA | 156 | GGTACATTCTGTGATAATAACAGGAGTCAG |
| | | ATTTGCAGTCCCTGTCCTCCAAATAGTTTCT |
| | | CCAGCGCAGGTGGACAAAGGACCTGTGAC |
| | | ATATGCAGGCAGTGTAAAGGTGTTTTCAAG |
| | | ACCAGGAAGGAGTGTTCCTCCACCAGCAA |
| | | TGCAGAGTGTGACTGCATTTCAGGATATCA |
| | | CTGCCTGGGGGCAGAATGCAGCATGTGTG |
| | | AACAGGATTGTAAACAAGGTCAAGAATTG |
| | | ACAAAAAAAGGTTGTAAAGACTGTTGCTT |
| | | TGGGACATTTAATGACCAGAAACGTGGCAT |
| | | CTGTCGACCCTGGACAAACTGTTCTTTGGA |
| | | TGGAAAGTCTGTGCTTGTGAATGGGACGA |
| | | AGGAGAGGGACGTGGTCTGCGGACCATCT |
| | | CCAGCCGACCTCTCTCCAGGAGCATCCTCT |
| | | GCGACCCCGCCTGCCCCTGCGAGAGAGCC |
| | | AGGACACTCTCCGCAGGAGCCCAGAGGGC |
| | | CCACAATCAAGCCCTGTCCTCCATGCAAAT |
| | | GCCCAGCACCTAACCTCTTGGGTGGACCAT |
| | | CCGTCTTCATCTTCCCTCCAAAGATCAAGG |
| | | ATGTACTCATGATCTCCCTGAGCCCCATAGT |
| | | CACATGTGTGGTGGTGGATGTGAGCGAGG |
| | | ATGACCCAGATGTCCAGATCAGCTGGTTTG |
| | | TGAACAACGTGGAAGTACACACAGCTCAG |
| | | ACACAAACCCATAGAGAGGATTACAACAG |
| | | TACTCTCCGGGTGGTCAGTGCCCTCCCCAT |
| | | CCAGCACCAGGACTGGATGAGTGGCAAGG |
| | | AGTTCAAATGCAAGGTCAACAACAAAGAC |
| | | CTCCCAGCGCCCATCGAGAGAACCATCTCA |
| | | AAACCCAAAGGGTCAGTAAGAGCTCCACA |
| | | GGTATATGTCTTGCCTCCACCAGAAGAAGA |
| | | GATGACTAAGAAACAGGTCACTCTGACCT |
| | | GCATGGTCACAGACTTCATGCCTGAAGAC |
| | | ATTTACGTGGAGTGGACCAACAACGGGAA |
| | | AACAGAGCTAAACTACAAGAACACTGAAC |
| | | CAGTCCTGGACTCTGATGGTTCTTACTTCA |
| | | TGTACAGCAAGCTGAGAGTGGAAAAGAAG |
| | | AACTGGGTGGAAAGAAATAGCTACTCCTG |
| | | TTCAGTGGTCCACGAGGGTCTGCACAATC |
| | | ACCACACGACTAAGAGCTTCTCCCGGACT |
| | | CCGGGTAAA |
| human CD40 AA | SEQ ID NO: | MVRLPLQCVLWGCLLTAVHPEPPTACREKQ |
| | 157 | YLINSQCCSLCQPGQKLVSDCTEFTETECLPC |
| | | GESEFLDTWNRETHCHQHKYCDPNLGLRVQ |
| | | QKGTSETDTICTCEEGWHCTSEACESCVLHR |
| | | SCSPGFGVKQIATGVSDTICEPCPVGFFSNVS |
| | | SAFEKCHPWTSCETKDLVVQQAGTNKTDVV |
| | | CGPQDRLRALVVIPIIFGILFAILLVLVFIKKVA |
| | | KKPTNKAPHPKQEPQEINFPDDLPGSNTAAP |
| | | VQETLHGCQPVTQEDGKESRISVQERQ |
| human CD40 DNA | SEQ ID NO: | ATGGTTCGTCTGCCTCTGCAGTGCGTCCTC |
| | 158 | TGGGGCTGCTTGCTGACCGCTGTCCATCCA |
| | | GAACCACCCACTGCATGCAGAGAAAAACA |
| | | GTACCTAATAAACAGTCAGTGCTGTTCTTT |
| | | GTGCCAGCCAGGACAGAAACTGGTGAGTG |
| | | ACTGCACAGAGTTCACTGAAACGGAATGC |
| | | CTTCCTTGCGGTGAAAGCGAATTCCTAGAC |
| | | ACCTGGAACAGAGAGACACACTGCCACCA |
| | | GCACAAATACTGCGACCCCAACCTAGGGC |
| | | TTCGGGTCCAGCAGAAGGGCACCTCAGAA |
| | | ACAGACACCATCTGCACCTGTGAAGAAGG |
| | | CTGGCACTGTACGAGTGAGGCCTGTGAGA |
| | | GCTGTGTCCTGCACCGCTCATGCTCGCCCG |
| | | GCTTTGGGGTCAAGCAGATTGCTACAGGG |
| | | GTTTCTGATACCATCTGCGAGCCCTGCCCA |
| | | GTCGGCTTCTTCTCCAATGTGTCATCTGCTT |
| | | TCGAAAAATGTCACCCTTGGACAAGCTGT |
| | | GAGACCAAAGACCTGGTTGTGCAACAGGC |
| | | AGGCACAAACAAGACTGATGTTGTCTGTG |
| | | GTCCCCAGGATCGGCTGAGAGCCCTGGTG |
| | | GTGATCCCCATCATCTTCGGGATCCTGTTTG |
| | | CCATCCTCTTGGTGCTGGTCTTTATCAAAA |
| | | AGGTGGCCAAGAAGCCAACCAATAAGGCC |
| | | CCCCACCCCAAGCAGGAACCCCAGGAGAT |
| | | CAATTTTCCCGACGATCTTCCTGGCTCCAA |
| | | CACTGCTGCTCCAGTGCAGGAGACTTTAC |
| | | ATGGATGCCAACCGGTCACCCAGGAGGAT |
| | | GGCAAAGAGAGTCGCATCTCAGTGCAGGA |
| | | GAGACAGTGA |
| huCD40 ECD AA | SEQ ID NO: | MVRLPLQCVLWGCLLTAVHPEPPTACREKQ |
| (1-194) | 159 | YLINSQCCSLCQPGQKLVSDCTEFTETECLPC |
| | | GESEFLDTWNRETHCHQHKYCDPNLGLRVQ |
| | | QKGTSETDTICTCEEGWHCTSEACESCVLHR |
| | | SCSPGFGVKQIATGVSDTICEPCPVGFFSNVS |
| | | SAFEKCHPWTSCETKDLVVQQAGTNKTDVV |
| | | CGPQDRLRA |
| huCD40 ECD | SEQ ID NO: | ATGGTTCGTCTGCCTCTGCAGTGCGTCCTC |
| DNA (1-194) | 160 | TGGGGCTGCTTGCTGACCGCTGTCCATCCA |
| | | GAACCACCCACTGCATGCAGAGAAAAACA |
| | | GTACCTAATAAACAGTCAGTGCTGTTCTTT |
| | | GTGCCAGCCAGGACAGAAACTGGTGAGTG |
| | | ACTGCACAGAGTTCACTGAAACGGAATGC |
| | | CTTCCTTGCGGTGAAAGCGAATTCCTAGAC |
| | | ACCTGGAACAGAGAGACACACTGCCACCA |
| | | GCACAAATACTGCGACCCCAACCTAGGGC |
| | | TTCGGGTCCAGCAGAAGGGCACCTCAGAA |
| | | ACAGACACCATCTGCACCTGTGAAGAAGG |
| | | CTGGCACTGTACGAGTGAGGCCTGTGAGA |
| | | GCTGTGTCCTGCACCGCTCATGCTCGCCCG |
| | | GCTTTGGGGTCAAGCAGATTGCTACAGGG |
| | | GTTTCTGATACCATCTGCGAGCCCTGCCCA |
| | | GTCGGCTTCTTCTCCAATGTGTCATCTGCTT |
| | | TCGAAAAATGTCACCCTTGGACAAGCTGT |
| | | GAGACCAAAGACCTGGTTGTGCAACAGGC |
| | | AGGCACAAACAAGACTGATGTTGTCTGTG |
| | | GTCCCCAGGATCGGCTGAGAGCC |
| human CD40 ECD- | SEQ ID NO: | MVRLPLQCVLWGCLLTAVHPEPPTACREKQ |
| mIgG2a AA | 161 | YLINSQCCSLCQPGQKLVSDCTEFTETECLPC |
| | | GESEFLDTWNRETHCHQHKYCDPNLGLRVQ |
| | | QKGTSETDTICTCEEGWHCTSEACESCVLHR |
| | | SCSPGFGVKQIATGVSDTICEPCPVGFFSNVS |
| | | SAFEKCHPWTSCETKDLVVQQAGTNKTDVV |
| | | CGPQDRLRASEPRGPTIKPCPPCKCPAPNLLG |
| | | GPSVFIFPPKIKDVLMISLSPMVTCVVVDVSE |
| | | DDPDVQISWFVNNVEVLTAQTQTHREDYNS |
| | | TLRVVSALPIQHQDWMSGKEFKCKVNNKAL |
| | | PAPIERTISKPKGSVRAPQVYVLPPPEEEMTK |
| | | KQVTLTCMVTDFMPEDIYVEWTNNGKTELN |
| | | YKNTEPVLDSDGSYFMYSKLRVEKKNWVE |
| | | RNSYSCSVVHEGLHNHHTTKSFSRTPGK |
| human CD40 ECD- | SEQ ID NO: | ATGGTTCGTCTGCCTCTGCAGTGCGTCCTC |
| mIgG2a DNA | 162 | TGGGGCTGCTTGCTGACCGCTGTCCATCCA |
| | | GAACCACCCACTGCATGCAGAGAAAAACA |
| | | GTACCTAATAAACAGTCAGTGCTGTTCTTT |
| | | GTGCCAGCCAGGACAGAAACTGGTGAGTG |
| | | ACTGCACAGAGTTCACTGAAACGGAATGC |
| | | CTTCCTTGCGGTGAAAGCGAGTTCCTAGAC |
| | | ACCTGGAACAGAGAGACACACTGCCACCA |
| | | GCACAAATACTGCGACCCCAACCTAGGGC |
| | | TTCGGGTCCAGCAGAAGGGCACCTCAGAA |
| | | ACAGACACCATCTGCACCTGTGAAGAAGG |
| | | CTGGCACTGTACGAGTGAGGCCTGTGAGA |
| | | GCTGTGTCCTGCACCGCTCATGCTCGCCCG |
| | | GCTTTGGGGTCAAGCAGATTGCTACAGGG |
| | | GTTTCTGATACCATCTGCGAGCCCTGCCCA |
| | | GTCGGCTTCTTCTCCAATGTGTCATCTGCTT |
| | | TCGAAAAATGTCACCCTTGGACAAGCTGT |
| | | GAGACCAAAGACCTGGTTGTGCAACAGGC |
| | | AGGCACAAACAAGACTGATGTTGTCTGTG |
| | | GTCCCCAGGATCGGCTGAGAGCTAGCGAG |
| | | CCCAGAGGGCCCACAATCAAGCCCTGTCC |
| | | TCCATGCAAATGCCCAGCACCTAACCTCTT |
| | | GGGTGGACCATCCGTCTTCATCTTCCCTCC |
| | | AAAGATCAAGGATGTACTCATGATCTCCCT |
| | | GAGCCCCATGGTCACATGTGTGGTGGTGG |
| | | ATGTGAGCGAGGATGACCCAGATGTCCAG |
| | | ATCAGCTGGTTCGTGAACAACGTGGAAGT |
| | | ACTCACAGCTCAGACACAAACCCATAGAG |
| | | AGGATTACAACAGTACTCTCCGGGTGGTCA |
| | | GTGCCCTCCCCATCCAGCACCAGGACTGG |
| | | ATGAGTGGCAAGGAGTTCAAATGCAAGGT |
| | | CAACAACAAAGCCCTCCCAGCGCCCATCG |
| | | AGAGAACCATCTCAAAACCCAAAGGGTCA |
| | | GTAAGAGCTCCACAGGTATATGTCTTGCCT |
| | | CCACCAGAAGAAGAGATGACTAAGAAACA |
| | | GGTCACTCTGACCTGCATGGTCACAGACTT |
| | | CATGCCTGAAGACATTTACGTGGAGTGGAC |
| | | CAACAACGGGAAAACAGAGCTAAACTACA |
| | | AGAACACTGAACCAGTCCTGGACTCTGAT |
| | | GGTTCTTACTTCATGTACAGCAAGCTGAGA |
| | | GTGGAAAAGAAGAACTGGGTGGAAAGAA |
| | | ATAGCTACTCCTGCTCAGTGGTCCACGAGG |
| | | GTCTGCACAATCACCACACGACTAAGAGC |
| | | TTCTCCCGGACTCCGGGTAAA |
| human OX40 AA | SEQ ID NO: | MCVGARRLGRGPCAALLLLGLGLSTVTGLH |
| | 163 | CVGDTYPSNDRCCHECRPGNGMVSRCSRSQ |
| | | NTVCRPCGPGFYNDVVSSKPCKPCTWCNLR |
| | | SGSERKQLCTATQDTVCRCRAGTQPLDSYKP |
| | | GVDCAPCPPGHFSPGDNQACKPWTNCTLAG |
| | | KHTLQPASNSSDAICEDRDPPATQPQETQGPP |
| | | ARPITVQPTEAWPRTSQGPSTRPVEVPGGRA |
| | | VAAILGLGLVLGLLGPLAILLALYLLRRDQRL |
| | | PPDAHKPPGGGSFRTPIQEEQADAHSTLAKI |
| human OX40 DNA | SEQ ID NO: | ATGTGCGTGGGGGCTCGGCGGCTGGGCCG |
| | 164 | CGGGCCGTGTGCGGCTCTGCTCCTCCTGGG |
| | | CCTGGGGCTGAGCACCGTGACGGGGCTCC |
| | | ACTGTGTCGGGGACACCTACCCCAGCAAC |
| | | GACCGGTGCTGCCACGAGTGCAGGCCAGG |
| | | CAACGGGATGGTGAGCCGCTGCAGCCGCT |
| | | CCCAGAACACGGTGTGCCGTCCGTGCGGG |
| | | CCGGGCTTCTACAACGACGTGGTCAGCTC |
| | | CAAGCCGTGCAAGCCCTGCACGTGGTGTA |
| | | ACCTCAGAAGTGGGAGTGAGCGGAAGCA |
| | | GCTGTGCACGGCCACACAGGACACAGTCT |
| | | GCCGCTGCCGGGCGGGCACCCAGCCCCTG |
| | | GACAGCTACAAGCCTGGAGTTGACTGTGC |
| | | CCCCTGCCCTCCAGGGCACTTCTCCCCAGG |
| | | CGACAACCAGGCCTGCAAGCCCTGGACCA |
| | | ACTGCACCTTGGCTGGGAAGCACACCCTG |
| | | CAGCCGGCCAGCAATAGCTCGGACGCAAT |
| | | CTGTGAGGACAGGGACCCCCCAGCCACGC |
| | | AGCCCCAGGAGACCCAGGGCCCCCCGGCC |
| | | AGGCCCATCACTGTCCAGCCCACTGAAGC |
| | | CTGGCCCAGAACCTCACAGGGACCCTCCA |
| | | CCCGGCCCGTGGAGGTCCCCGGGGGCCGT |
| | | GCGGTTGCCGCCATCCTGGGCCTGGGCCTG |
| | | GTGCTGGGGCTGCTGGGCCCCCTGGCCATC |
| | | CTGCTGGCCCTGTACCTGCTCCGGAGGGA |
| | | CCAGAGGCTGCCCCCCGATGCCCACAAGC |
| | | CCCCTGGGGGAGGCAGTTTCCGGACCCCC |
| | | ATCCAAGAGGAGCAGGCCGACGCCCACTC |
| | | CACCCTGGCCAAGATCTGA |
| human OX40 ECD | SEQ ID NO: | MCVGARRLGRGPCAALLLLGLGLSTVTGLH |
| (1-216) AA | 165 | CVGDTYPSNDRCCHECRPGNGMVSRCSRSQ |
| | | NTVCRPCGPGFYNDVVSSKPCKPCTWCNLR |
| | | SGSERKQLCTATQDTVCRCRAGTQPLDSYKP |
| | | GVDCAPCPPGHFSPGDNQACKPWTNCTLAG |
| | | KHTLQPASNSSDAICEDRDPPATQPQETQGPP |
| | | ARPITVQPTEAWPRTSQGPSTRPVEVPGGRA |
| | | VA |
| human OX40 ECD | SEQ ID NO: | ATGTGCGTGGGGGCTCGGCGGCTGGGCCG |
| (1-216) DNA | 166 | CGGGCCGTGTGCGGCTCTGCTCCTCCTGGG |
| | | CCTGGGGCTGAGCACCGTGACGGGGCTCC |
| | | ACTGTGTCGGGGACACCTACCCCAGCAAC |
| | | GACCGGTGCTGCCACGAGTGCAGGCCAGG |
| | | CAACGGGATGGTGAGCCGCTGCAGCCGCT |
| | | CCCAGAACACGGTGTGCCGTCCGTGCGGG |
| | | CCGGGCTTCTACAACGACGTGGTCAGCTC |
| | | CAAGCCGTGCAAGCCCTGCACGTGGTGTA |
| | | ACCTCAGAAGTGGGAGTGAGCGGAAGCA |
| | | GCTGTGCACGGCCACACAGGACACAGTCT |
| | | GCCGCTGCCGGGCGGGCACCCAGCCCCTG |
| | | GACAGCTACAAGCCTGGAGTTGACTGTGC |
| | | CCCCTGCCCTCCAGGGCACTTCTCCCCAGG |
| | | CGACAACCAGGCCTGCAAGCCCTGGACCA |
| | | ACTGCACCTTGGCTGGGAAGCACACCCTG |
| | | CAGCCGGCCAGCAATAGCTCGGACGCAAT |
| | | CTGTGAGGACAGGGACCCCCCAGCCACGC |
| | | AGCCCCAGGAGACCCAGGGCCCCCCGGCC |
| | | AGGCCCATCACTGTCCAGCCCACTGAAGC |
| | | CTGGCCCAGAACCTCACAGGGACCCTCCA |
| | | CCCGGCCCGTGGAGGTCCCCGGGGGCCGT |
| | | GCGGTTGCC |
| human OX40 ECD- | SEQ ID NO: | MCVGARRLGRGPCAALLLLGLGLSTVTGLH |
| His AA | 167 | CVGDTYPSNDRCCHECRPGNGMVSRCSRSQ |
| | | NTVCRPCGPGFYNDVVSSKPCKPCTWCNLR |
| | | SGSERKQLCTATQDTVCRCRAGTQPLDSYKP |
| | | GVDCAPCPPGHFSPGDNQACKPWTNCTLAG |
| | | KHTLQPASNSSDAICEDRDPPATQPQETQGPP |
| | | ARPITVQPTEAWPRTSQGPSTRPVEVPGGRA |
| | | VAHHHHHH |
| human OX40 ECD- | SEQ ID NO: | ATGTGCGTGGGGGCTCGGCGGCTGGGCCG |
| His DNA | 168 | CGGGCCGTGTGCGGCTCTGCTCCTCCTGGG |
| | | CCTGGGGCTGAGCACCGTGACGGGGCTCC |
| | | ACTGTGTCGGGGACACCTACCCCAGCAAC |
| | | GACCGGTGCTGCCACGAGTGCAGGCCAGG |
| | | CAACGGGATGGTGAGCCGCTGCAGCCGCT |
| | | CCCAGAACACGGTGTGCCGTCCGTGCGGG |
| | | CCGGGCTTCTACAACGACGTGGTCAGCTC |
| | | CAAGCCGTGCAAGCCCTGCACGTGGTGTA |
| | | ACCTCAGAAGTGGGAGTGAGCGGAAGCA |
| | | GCTGTGCACGGCCACACAGGACACAGTCT |
| | | GCCGCTGCCGGGCGGGCACCCAGCCCCTG |
| | | GACAGCTACAAGCCTGGAGTTGACTGTGC |
| | | CCCCTGCCCTCCAGGGCACTTCTCCCCAGG |
| | | CGACAACCAGGCCTGCAAGCCCTGGACCA |
| | | ACTGCACCTTGGCTGGGAAGCACACCCTG |
| | | CAGCCGGCCAGCAATAGCTCGGACGCAAT |
| | | CTGTGAGGACAGGGACCCCCCAGCCACGC |
| | | AGCCCCAGGAGACCCAGGGCCCCCCGGCC |
| | | AGGCCCATCACTGTCCAGCCCACTGAAGC |
| | | CTGGCCCAGAACCTCACAGGGACCCTCCA |
| | | CCCGGCCCGTGGAGGTCCCCGGGGGCCGT |
| | | GCGGTTGCCCATCACCATCACCATCAC |

### Example 16. Generation of Anti-huCD137 VH Domain Antibodies

### Construction of Synthetic Human VH Antibody Repertoires

Synthetic libraries were constructed essentially using the germline 3-23 (SEQ ID NO: 169 and 170). Randomization of heavy chain CDRs (HCDRs) was carried out by combinatorial mutagenesis using degenerate oligonucleotides. Randomization of the HCDR1 and HCDR2 regions was carried out via multiple site-specific mutations by polymerase chain reaction as described by Meetei (Meetei et al., (1998) Anal. Biochem, 264, 288-91; Meetei et al., (2002) Methods Mol Biol, 182, 95-102). For CDR3 regions, different lengths from 8 to 14 (Kabat definition) of degenerate oligonucleotides were synthesized (Invitrogen), and diversity was introduced by splice-overlap extension PCR. The PCR products after the mutagenesis steps, were double-digested by NcoI/NotI and ligated into the phagemid vector pCANTAB-5E. Repertoires were then transformed into Escherichia coli TG1 bacteria and validated by DNA Sanger sequencing of random clones (>96 clones analyzed). Phages were purified by two precipitations with PEG/NaCl directly from the culture supernatant after a rescue step using KM13 helper phage. A library with a total size of 1.38×10¹¹ was obtained after transformation into E. coli bacteria.

TABLE 18
Germline used for library construction

| | | |
|---|---|---|
| 3-23 germline AA | SEQ ID NO: 169 | EVQLLESGGGLVQPGGSLRLSCAASGFTFSSY |
| | | AISWVRQAPGKGLEWVSAISGSGGSTYYADS |
| | | VKGRFTISRDNSKNTLYLQMNSLRAEDTAVY |
| | | YCAR |
| 3-23 germline | SEQ ID NO: 170 | GAAGTGCAGCTGCTGGAAAGCGGCGGCGG |
| DNA | | CCTGGTGCAGCCGGGCGGCAGCCTGCGCCT |
| | | GAGCTGCGCGGCGAGCGGCTTTACCTTTAG |
| | | CAGCTATGCGATTAGCTGGGTGCGCCAGGC |
| | | GCCGGGCAAAGGCCTGGAATGGGTGAGCG |
| | | CGATTAGCGGCAGCGGCGGTAGCACCTATT |
| | | ATGCGGATAGCGTGAAAGGCCGCTTTACCA |
| | | TTAGCCGCGATAACAGCAAAAACACCCTGT |
| | | ATCTGCAGATGAACAGCCTGCGCGCGGAAG |
| | | ATACCGCCGTGTATTATTGCGCGCGT |

### Phage Display Panning and Screening

Phage display selection was carried out by phage display using standard protocols (Silacci et al., (2005) Proteomics, 5, 2340-50; Zhao et al., (2014) PLoS One, 9, e111339). In brief, 10 μg/ml of immobilized human CD137 ECD-mIgG2a in immunotubes (Cat. 470319, ThermoFisher) was utilized in round 1 and 2. Hut78/huCD137 cells were used for selection in round 3 and 4. Immunotubes were blocked with 5% milk powder (w/v) in PBS supplemented with 1% Tween 20 (MPBST) for 1 hour. After washes with PBST (PBS buffer supplemented with 0.05% Tween 20), 5×10¹² (round 1) or 5×10¹¹(rounds 2) phages from each sub library were depleted by human CD40 ECD-mIgG2a in MPBST for 1 hour and then incubated with the antigen for 1 hour. For the third and fourth rounds of selections, cell panning was carried out using Hut78/huCD137 cells (round 3) with HEK293(ATCC, CRL-1573) cells as depletion cells. After washes with PBST, bound phages were eluted with 100 mM triethylamine (Sigma-Aldrich). Eluted phages were used to infect mid-log phase E. coli TG1 bacteria and plated onto TYE-agar plates supplemented with 2% glucose and 100 μg/ml ampicillin. After four rounds of selections, individual clones were picked up and phage containing supernatants were prepared using standard protocols. Phage ELISA and FACS were used to screen anti-huCD137 VH domain antibodies.

For phage ELISA, a Maxisorp™ immunoplate was coated with antigens and blocked with 5% milk powder (w/v) in PBS buffer. Phage supernatant was blocked with MPBST for 30 min and added to wells of the ELISA plate for 1 hour. After washes with PBST, bound phage was detected using HRP-conjugated anti-M13 antibody (GE Healthcare) and 3,3′,5,5′-tetramethylbenzidine substrate (Cat.: 00-4201-56, eBioscience, USA). The ELISA-positive clones were further verified by flow cytometry using Hut78/huCD137 cells. CD137-expressing cells (10⁵ cells/well) were incubated with ELISA-positive phage supernatants, followed by binding with Alexa Fluro-647-labeled anti-M13 antibody (GE Healthcare). Cell fluorescence was quantified using a flow cytometer (Guava easyCyte™ 8HT, Merck-Millipore, USA).

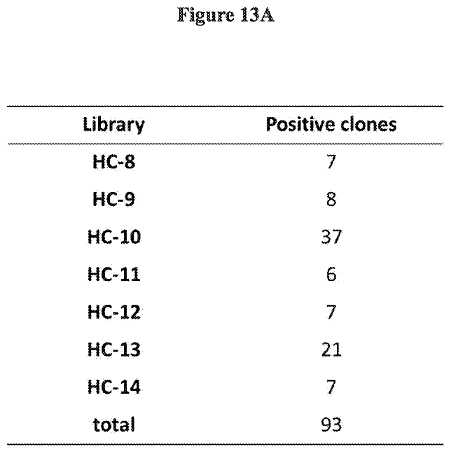

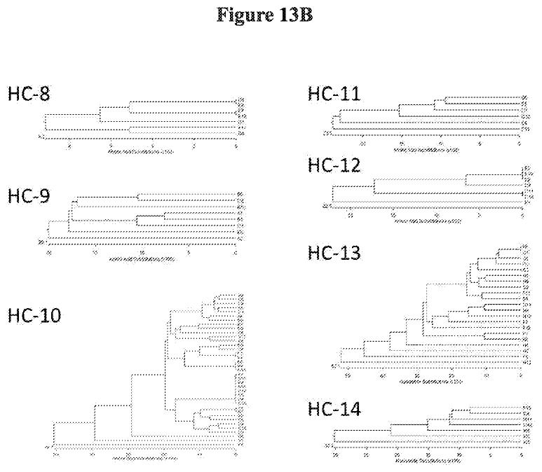

The clones that showed positive signals in FACS screening, and binding to both huCD137 and cynoCD137 but not huOX40 and huCD40, were picked up and sequenced. Approximately 76 unique sequences from 93 positive clones were identified (FIG. 13A-13B).

### Expression and Purification of Fc Fusion VH Antibodies

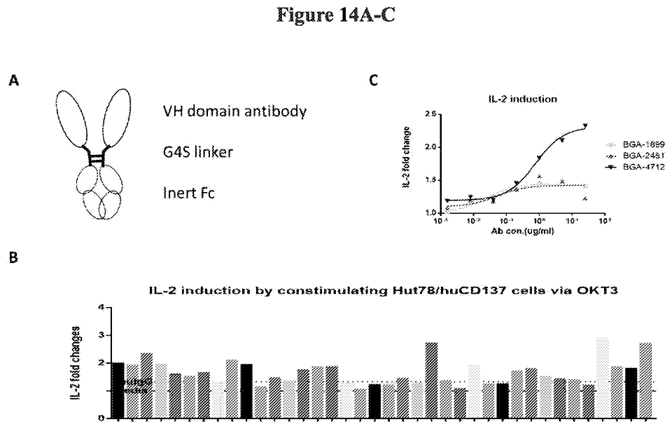

The VH sequences were analyzed by comparing sequence homology and grouped based on sequence similarity. Complementary determining regions (CDRs) were defined based on the Kabat (Wu and Kabat (1970) J. Exp. Med. 132:211-250) and IMGT (Lefranc (1999) Nucleic Acids Research 27:209-212) system by sequence annotation and by internet-based sequence analysis. The amino acid and DNA sequences of two representative top clones BGA-7207 and BGA-4712 are listed in Table 19 below. After the sequence checking and analysis of binding curve by SPR, anti-huCD137 VH domain antibodies were then constructed as human Fc fusion VH antibody format (VH-Fc) using in-house developed expression vectors. As shown in FIG. 14A, VH domain antibodies were fused at the N terminal of human Fc with a G4S (SEQ ID NO: 324) linker in between. A Fc-null version (an inert Fc without FcγR-binding) of human IgG1 (SEQ ID NO: 175) was used. Expression and preparation of Fc fusion VH antibodies were achieved by transfection into 293G cells and by purification using a Protein A column (Cat. No. 17543802, GE Life Sciences). The purified antibodies were concentrated to 0.5-5 mg/mL in PBS and stored in aliquots in a −80° C. freezer.

TABLE 19
Amino acid and DNA sequences of two selected anti-huCD137 VH domain
antibodies

| Antibody | SEQ ID NO | | SEQUENCE |
|---|---|---|---|
| BGA-7207 | SEQ ID NO: 60 | VH | EVQLLESGGGLVQPGGSLRLSCAASGFR |
| | | | LDTTEVGWVRQAPGKGLEWVSTIMGIS |
| | | | GSTYYADSVKGRFTISRDNSKNTLYLQM |
| | | | NSLRAEDTAVYYCARVVDSLFDDSAVED |
| | | | YWGQGTLVTVSS |
| | SEQ ID NO: 61 | VH DNA | GAGGTCCAGTTACTTGAGAGTGGTGG |
| | | | AGGTCTGGTCCAACCAGGAGGTTCGC |
| | | | TGCGTTTATCCTGCGCCGCGTCTGGATT |
| | | | CAGATTGGACACCACCGAAGTAGGTT |
| | | | GGGTGCGTCAAGCGCCGGGGAAAGGA |
| | | | CTGGAATGGGTCTCCACCATCATGGGT |
| | | | ATTAGTGGTTCGACATACTATGCGGAC |
| | | | AGTGTCAAAGGGCGCTTTACGATCTCG |
| | | | CGCGATAACTCAAAAAATACTCTTTAC |
| | | | CTTCAAATGAATAGCCTTCGTGCTGAG |
| | | | GACACTGCGGTGTACTACTGCGCCCGC |
| | | | GTCGTCGATTCCCTCTTTGATGACAGC |
| | | | GCCGTTTTTGATTACTGGGGACAGGGC |
| | | | ACCTTAGTTACAGTCTCATCG |
| | SEQ ID NO: | VH-Fc | EVQLLESGGGLVQPGGSLRLSCAASGFR |
| | 171 | | LDTTEVGWVRQAPGKGLEWVSTIMGIS |
| | | | GSTYYADSVKGRFTISRDNSKNTLYLQM |
| | | | NSLRAEDTAVYYCARVVDSLFDDSAVFD |
| | | | YWGQGTLVTVSSGGGGSDKTHTCPPCP |
| | | | APPAAGPSVFLFPPKPKDTLMISRTPEVT |
| | | | CVVVDVSHEDPEVKFNWYVDGVEVHN |
| | | | AKTKPREEQYNSTYRVVSVLTVLHQDW |
| | | | LNGKEYKCKVSNKALAAPIEKTISKAKG |
| | | | QPREPQVYTLPPSREEMTKNQVSLTCLV |
| | | | KGFYPSDIAVEWESNGQPENNYKTTPPV |
| | | | LDSDGSFFLYSKLTVDKSRWQQGNVFSC |
| | | | SVMHEALHNHYTQKSLSLSPGK |
| | SEQ ID NO: | VH-Fc DNA | GAGGTCCAGTTACTTGAGAGTGGTGG |
| | 172 | | AGGTCTGGTCCAACCAGGAGGTTCGC |
| | | | TGCGTTTATCCTGCGCCGCGTCTGGATT |
| | | | CAGATTGGACACCACCGAAGTAGGTT |
| | | | GGGTGCGTCAAGCGCCGGGGAAAGGA |
| | | | CTGGAATGGGTCTCCACCATCATGGGT |
| | | | ATTAGTGGTTCGACATACTATGCGGAC |
| | | | AGTGTCAAAGGGCGCTTTACGATCTCG |
| | | | CGCGATAACTCAAAAAATACTCTTTAC |
| | | | CTTCAAATGAATAGCCTTCGTGCTGAG |
| | | | GACACTGCGGTGTACTACTGCGCCCGC |
| | | | GTCGTCGATTCCCTCTTTGATGACAGC |
| | | | GCCGTTTTTGATTACTGGGGACAGGGC |
| | | | ACCTTAGTTACAGTCTCATCGGGCGGC |
| | | | GGAGGGTCTGACAAGACCCACACATG |
| | | | CCCCCCTTGTCCTGCTCCACCAGCTGC |
| | | | AGGACCAAGCGTGTTCCTGTTTCCACC |
| | | | CAAGCCCAAGGATACCCTGATGATCTC |
| | | | TCGGACCCCAGAGGTGACATGCGTGG |
| | | | TGGTGGATGTGAGCCACGAGGACCCC |
| | | | GAGGTGAAGTTCAACTGGTATGTGGAC |
| | | | GGCGTGGAGGTGCACAATGCTAAGAC |
| | | | CAAGCCCAGGGAGGAGCAGTACAACT |
| | | | CCACCTATAGAGTGGTGTCTGTGCTGA |
| | | | CAGTGCTGCACCAGGATTGGCTGAAC |
| | | | GGCAAGGAGTATAAGTGCAAGGTGTC |
| | | | CAATAAGGCCCTGGCCGCTCCTATCGA |
| | | | GAAGACCATCTCTAAGGCCAAGGGCC |
| | | | AGCCCAGAGAGCCTCAGGTGTACACA |
| | | | CTGCCTCCATCCCGGGAAGAGATGACC |
| | | | AAGAACCAGGTGTCTCTGACATGTCTG |
| | | | GTCAAGGGCTTCTATCCCTCTGACATC |
| | | | GCCGTGGAGTGGGAGAGCAATGGCCA |
| | | | GCCTGAGAACAATTACAAGACCACAC |
| | | | CCCCTGTGCTGGATTCCGACGGCTCTT |
| | | | TCTTTCTGTATAGCAAGCTGACCGTGG |
| | | | ACAAGTCCCGGTGGCAGCAGGGCAAC |
| | | | GTGTTCAGCTGTTCCGTGATGCACGAA |
| | | | GCTCTGCATAATCACTATACTCAGAAAT |
| | | | CCCTGTCACTGTCACCTGGTAAA |
| BGA-4712 | SEQ ID NO: 70 | VH | EVQLLESGGGLVQPGGSLRLSCAASGF |
| | | | MLSAEDVGWVRQAPGKGLEWVSAILD |
| | | | FGGSTYYADSVKGRFTISRDNSKNTLYL |
| | | | QMNSLRAEDTAVYYCARVVYHAGGGV |
| | | | TFDYWGQGTLVTVSS |
| | SEQ ID NO: 71 | VH DNA | GAGGTCCAGTTACTTGAGAGTGGTGG |
| | | | AGGTCTGGTCCAACCAGGAGGTTCGC |
| | | | TGCGTTTATCCTGCGCCGCGTCTGGATT |
| | | | CATGTTGTCCGCCGAAGACGTGGGTTG |
| | | | GGTGCGTCAAGCGCCGGGGAAAGGAC |
| | | | TGGAATGGGTCTCCGCCATCTTGGATT |
| | | | TTGGTGGTTCGACATACTATGCGGACA |
| | | | GTGTCAAAGGGCGCTTTACGATCTCGC |
| | | | GCGATAACTCAAAAAATACTCTTTACC |
| | | | TTCAAATGAATAGCCTTCGTGCTGAGG |
| | | | ACACTGCGGTGTACTACTGCGCCCGCG |
| | | | TCGTCTACCATGCTGGTGGTGGCGTCA |
| | | | CCTTTGATTACTGGGGACAGGGCACCT |
| | | | TAGTTACAGTCTCATCG |
| | SEQ ID NO: | VH-Fc | EVQLLESGGGLVQPGGSLRLSCAASGF |
| | 173 | | MLSAEDVGWVRQAPGKGLEWVSAILD |
| | | | FGGSTYYADSVKGRFTISRDNSKNTLYL |
| | | | QMNSLRAEDTAVYYCARVVYHAGGGV |
| | | | TFDYWGQGTLVTVSSGGGGSDKTHTCP |
| | | | PCPAPPAAGPSVFLFPPKPKDTLMISRTP |
| | | | EVTCVVVDVSHEDPEVKFNWYVDGVE |
| | | | VHNAKTKPREEQYNSTYRVVSVLTVLH |
| | | | QDWLNGKEYKCKVSNKALAAPIEKTIS |
| | | | KAKGQPREPQVYTLPPSREEMTKNQVS |
| | | | LTCLVKGFYPSDIAVEWESNGQPENNYK |
| | | | TTPPVLDSDGSFFLYSKLTVDKSRWQQG |
| | | | NVFSCSVMHEALHNHYTQKSLSLSPGK |
| | SEQ ID NO: | VH-Fc DNA | GAGGTCCAGTTACTTGAGAGTGGTGG |
| | 174 | | AGGTCTGGTCCAACCAGGAGGTTCGC |
| | | | TGCGTTTATCCTGCGCCGCGTCTGGATT |
| | | | CATGTTGTCCGCCGAAGACGTGGGTTG |
| | | | GGTGCGTCAAGCGCCGGGGAAAGGAC |
| | | | TGGAATGGGTCTCCGCCATCTTGGATT |
| | | | TTGGTGGTTCGACATACTATGCGGACA |
| | | | GTGTCAAAGGGCGCTTTACGATCTCGC |
| | | | GCGATAACTCAAAAAATACTCTTTACC |
| | | | TTCAAATGAATAGCCTTCGTGCTGAGG |
| | | | ACACTGCGGTGTACTACTGCGCCCGCG |
| | | | TCGTCTACCATGCTGGTGGTGGCGTCA |
| | | | CCTTTGATTACTGGGGACAGGGCACCT |
| | | | TAGTTACAGTCTCATCGGGCGGCGGAG |
| | | | GGTCTGACAAGACCCACACATGCCCC |
| | | | CCTTGTCCTGCTCCACCAGCTGCAGGA |
| | | | CCAAGCGTGTTCCTGTTTCCACCCAAG |
| | | | CCCAAGGATACCCTGATGATCTCTCGG |
| | | | ACCCCAGAGGTGACATGCGTGGTGGT |
| | | | GGATGTGAGCCACGAGGACCCCGAGG |
| | | | TGAAGTTCAACTGGTATGTGGACGGCG |
| | | | TGGAGGTGCACAATGCTAAGACCAAG |
| | | | CCCAGGGAGGAGCAGTACAACTCCAC |
| | | | CTATAGAGTGGTGTCTGTGCTGACAGT |
| | | | GCTGCACCAGGATTGGCTGAACGGCA |
| | | | AGGAGTATAAGTGCAAGGTGTCCAATA |
| | | | AGGCCCTGGCCGCTCCTATCGAGAAGA |
| | | | CCATCTCTAAGGCCAAGGGCCAGCCCA |
| | | | GAGAGCCTCAGGTGTACACACTGCCTC |
| | | | CATCCCGGGAAGAGATGACCAAGAAC |
| | | | CAGGTGTCTCTGACATGTCTGGTCAAG |
| | | | GGCTTCTATCCCTCTGACATCGCCGTG |
| | | | GAGTGGGAGAGCAATGGCCAGCCTGA |
| | | | GAACAATTACAAGACCACACCCCCTGT |
| | | | GCTGGATTCCGACGGCTCTTTCTTTCT |
| | | | GTATAGCAAGCTGACCGTGGACAAGTC |
| | | | CCGGTGGCAGCAGGGCAACGTGTTCA |
| | | | GCTGTTCCGTGATGCACGAAGCTCTGC |
| | | | ATAATCACTATACTCAGAAATCCCTGTC |
| | | | ACTGTCACCTGGTAAA |
| Inert Fc | SEQ ID NO: | A Fc-null | DKTHTCPPCPAPPAAGPSVFLFPPKPKDT |
| | 175 | version | LMISRTPEVTCVVVDVSHEDPEVKFNW |
| | | IgG1 | YVDGVEVHNAKTKPREEQYNSTYRVVS |
| | | | VLTVLHQDWLNGKEYKCKVSNKALAA |
| | | | PIEKTISKAKGQPREPQVYTLPPSREEMT |
| | | | KNQVSLTCLVKGFYPSDIAVEWESNGQP |
| | | | ENNYKTTPPVLDSDGSFFLYSKLTVDKS |
| | | | RWQQGNVFSCSVMHEALHNHYTQKSL |
| | | | SLSPGK |
| | SEQ ID NO: | A Fc-null | GACAAGACCCACACATGCCCCCCTTGT |
| | 176 | version | CCTGCTCCACCAGCTGCAGGACCAAG |
| | | IgG1 DNA | CGTGTTCCTGTTTCCACCCAAGCCCAA |
| | | | GGATACCCTGATGATCTCTCGGACCCC |
| | | | AGAGGTGACATGCGTGGTGGTGGATG |
| | | | TGAGCCACGAGGACCCCGAGGTGAAG |
| | | | TTCAACTGGTATGTGGACGGCGTGGAG |
| | | | GTGCACAATGCTAAGACCAAGCCCAG |
| | | | GGAGGAGCAGTACAACTCCACCTATAG |
| | | | AGTGGTGTCTGTGCTGACAGTGCTGCA |
| | | | CCAGGATTGGCTGAACGGCAAGGAGT |
| | | | ATAAGTGCAAGGTGTCCAATAAGGCCC |
| | | | TGGCCGCTCCTATCGAGAAGACCATCT |
| | | | CTAAGGCCAAGGGCCAGCCCAGAGAG |
| | | | CCTCAGGTGTACACACTGCCTCCATCC |
| | | | CGGGAAGAGATGACCAAGAACCAGGT |
| | | | GTCTCTGACATGTCTGGTCAAGGGCTT |
| | | | CTATCCCTCTGACATCGCCGTGGAGTG |
| | | | GGAGAGCAATGGCCAGCCTGAGAACA |
| | | | ATTACAAGACCACACCCCCTGTGCTGG |
| | | | ATTCCGACGGCTCTTTCTTTCTGTATAG |
| | | | CAAGCTGACCGTGGACAAGTCCCGGT |
| | | | GGCAGCAGGGCAACGTGTTCAGCTGT |
| | | | TCCGTGATGCACGAAGCTCTGCATAAT |
| | | | CACTATACTCAGAAATCCCTGTCACTG |
| | | | TCACCTGGTAAA |

### Example 17. Functional Screening of Anti-huCD137 VH Domain Antibodies

Functional screening was applied to selected anti-huCD137 VH domain antibodies with strong agonism using supernatant containing VH-Fc proteins. In brief, the 96-well white/clear bottom plates (Thermo Fisher) were pre-incubated with 3 ug/ml anti-hu CD3 (Invitrogen, Cat. No. 16-0037-85) at 50 μl/well for 5 min and then washed away by PBS buffer. Next, Hut78/huCD137 cells were resuspended at 5×10⁵ cells/ml, and directly plated into the pre-coated plates at 50 μl/well (25,000 well per well). Supernatants containing various VH-Fc proteins were mixed with the cells. Alternatively, for purified VH domain antibodies with Fc fusion, a dose titration of purified VH-Fc protein preparations was added in duplicate at 25, 5, 1, 0.2, 0.04, 0.008 or 0.0016 μg/ml at 50 μl/well. As a crosslinker, goat anti-hu IgG(H&L) polystyrene particles (6.46 μm) (Cat. No. HUP-60-5, Spherotech) were added. Assay plates were incubated overnight at 37° C., and the concentrations of IL-2 were measured after 24 hours. Data was plotted as IL-2 fold increase compared with the concentration in the well with media only. FIG. 14B shows a representative screening result using supernatants containing VH-Fc proteins, and one of the clones, BGA-4712 has been shown to be capable to stimulate IL-2 production in Hut78/huCD137 cells in a dose dependent manner (FIG. 14C).

### Example 18. Characterization of Purified Anti-huCD137 VH Domain Antibodies

### Characterization of Purified Antibodies Via ELISA

For antigen ELISA, a Maxisorp™ immunoplate was coated with antigens and blocked with 3% BSA (w/v) in PBS buffer (blocking buffer). Monoclonal VH domain antibodies were blocked with blocking buffer for 30 minutes and added to wells of the ELISA plate for 1 hour. After washes with PBST, bound antibodies were detected using HRP-conjugated anti-human IgG antibody (Sigma, A0170) and 3,3′,5,5′-tetramethylbenzidine substrate (Cat.: 00-4201-56, eBioscience, USA). All selected clones were shown to cross-react with cynoCD137 with no binding to human OX40 ECD and human CD40 ECD.

### Characterization of Purified Antibodies Via SPR Analysis

Characterization of anti-huCD137 VH domain antibodies were made by SPR assays using BIAcore™ T-200 (GE Life Sciences). Briefly, anti-human IgG (Fc) antibody was immobilized on an activated CM5 biosensor chip (Cat.: BR100839, GE Life Sciences). Anti-huCD137 domain antibodies were flowed over the chip surface and captured by anti-human IgG (Fc) antibody. Then a serial dilution (6.0 nM to 2150 nM) of human CD137 ECD-mIgG2a was flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ.

### Characterization of Purified Antibodies Via Flow Cytometry

For flow cytometry, human CD137+ expressing cells (10⁵ cells/well) were incubated with various concentrations of purified VH domain antibodies, followed by binding with Alexa Fluro-647-labeled anti-hu IgG Fc antibody (Cat.: 409320, BioLegend, USA). Cell fluorescence was quantified using a flow cytometer (Guava easyCyte™ 8HT, Merck-Millipore, USA). Ligand competition was also applied in a flow cytometry based assay. In brief, Hut78/huCD137 was incubated with Fc fusion VH domain antibodies (VH-Fc) in the presence of serially diluted human CD137 ligand-mIgG2a, followed by detection with Alexa Fluro-647-labeled anti-hu IgG Fc antibody (Cat.: 409320, BioLegend, USA).

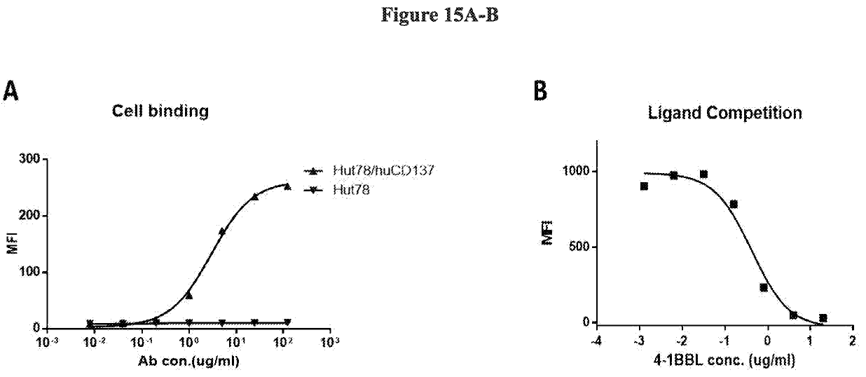

Selected VH domain antibodies were then characterized for affinity, cell binding and ligand competition. The SPR study, FACS analysis and the ligand competition result of one representative top clone BGA-4712 are shown in FIG. 15A-15B.

### Example 19. Construction of CEA×CD137 Multispecific Antibodies Using Anti-CD137 VH Domain Antibody BGA-4712 and an Anti-CEA Antibody

To explore potential more effective CD137 based mechanisms of action (MOAs) than single antibody treatment, a number of multispecific formats utilizing the anti-huCD137 VH domain antibody have been constructed and tested. Here, multiple formats have been adopted to create CD137-based T cell-engagers (TCEs), in which a first antigen binding domain is directed against a tumor-associated antigen (TAA) and a second antigen binding domain targets a CD137 activating receptor. For example, a first antigen binding domain of an anti-CEA antibody BGA-113 (SEQ ID NO: 179 and 181) was used to pair with a second antigen binding domain of an anti-huCD137 VH domain antibody BGA-4712 (SEQ ID NO: 70) in specifically defined formats as shown below(Table 20). For this construct, an inert Fc was used (SEQ ID NO: 175). Expression and preparation of these multispecific antibodies were achieved by transfection into 293G cells and by purification using a Protein A column (Cat. No. 17543802, GE Life Sciences). The purified antibodies were concentrated to 0.5-5 mg/mL in PBS and stored in aliquots in −80° C. freezer.

### Format A (A-CD137/CEA)

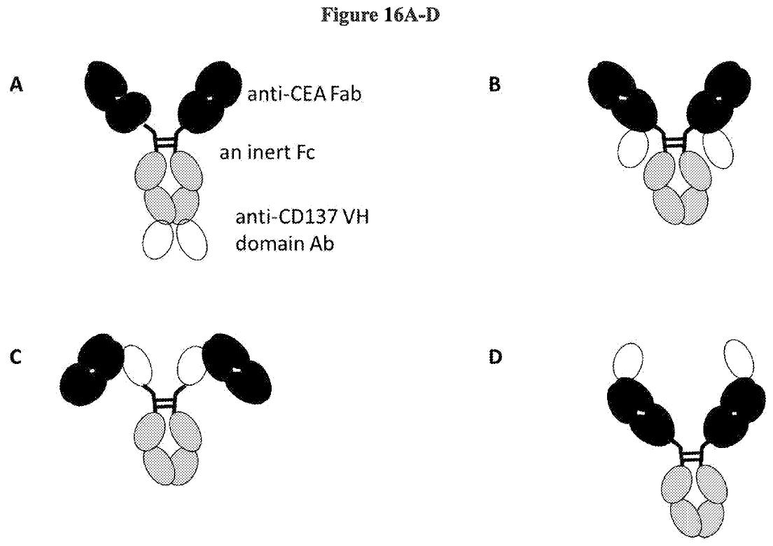

The format A provides a symmetric IgG-like multispecific molecule with Fab×VH configuration. Anti-huCD137 VH domain antibody BGA-4712 was fused to the c-termini of Fc (CH3 domain) of an anti-CEA antibody with one G4S linker (SEQ ID NO: 324) in between (SEQ ID NOs: 179, and 177) as shown in FIG. 16A.

### Format B (B-CD137/CEA)

The format B also provides a symmetric IgG-like multispecific molecule with Fab×VH configuration. Anti-huCD137 VH domain antibody BGA-4712 was fused to the c-termini of light chain (Cκ) of an anti-CEA antibody with one G4S linker (SEQ ID NO: 324) in between (SEQ ID NOs: 181 and 183) as shown in FIG. 16B.

### Format C (C-CD137/CEA)

The format C provides a symmetric VH antibody-like multispecific molecule with Fab×VH configuration. The Fab region of an anti-CEA antibody was fused to the N-termini of VH of anti-huCD137 VH domain antibody BGA-4712 with one G4S linker (SEQ ID NO: 324) in between (SEQ ID NOs: 179 and 185) as shown in FIG. 16C.

### Format D (D-CD137/CEA)

The format D also provides a symmetric IgG-like multispecific molecule with Fab×VH configuration. Anti-huCD137 VH domain antibody BGA-4712 was fused to the N-termini of heavy chain (Vh) of an anti-CEA antibody with one G4S linker (SEQ ID NO: 324) in between (SEQ ID NOs: 179 and 187) as shown in FIG. 16D.

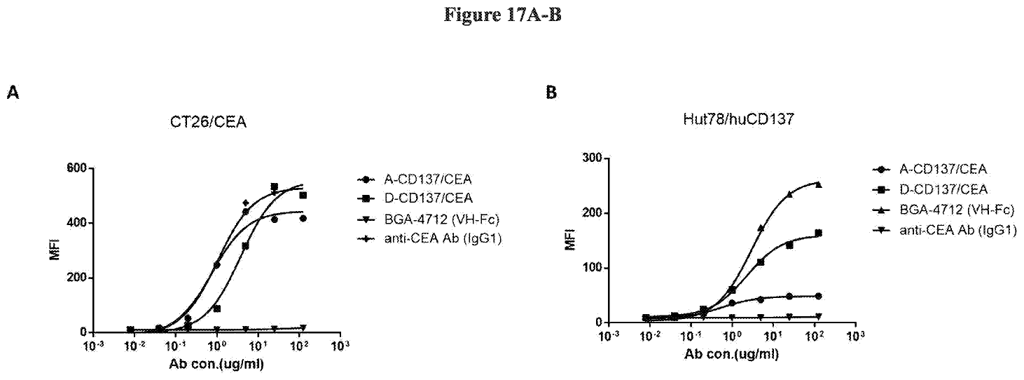

The yields and biochemical properties of various CD137/CEA multi specific antibodies were summarized in Table 21. For the two molecules A-CD137/CEA and D-CD137/CEA, the monomers are both above 95% based on the SEC-HPLC profiles (Table 21). By a flow cytometry-based assay, it was demonstrated that there is very low reduction of the affinity of the anti-CEA arm in the format A, whereas there was a significant affinity reduction of the anti-CEA arm in format D (FIG. 17A). It was also demonstrated that there was an affinity reduction of CD137 arm in the format A, whereas little or no influence on affinity in the format D (FIG. 17D).

TABLE 20
Amino acid and DNA sequences of CEAxCD137 multispecific antibodies of
various formats

| | | SEQ ID | |
|---|---|---|---|
| CONSTRUCT | Chain | NO | SEQUENCE |
| A-CD137/CEA | A-BGA- | SEQ ID | QVQLVQSGAEVKKPGASVKVSCKASGYIFT |
| | 4712/CEA heavy | NO: 177 | SYYLHWVRQAPGQGLEWIGYINPQTGKTS |
| | chain AA | | YAQKFQGRVTMTRDTSTSTVYMELSSLRSE |
| | | | DTAVYYCAREYGNYNYPLDYWGQGTLVT |
| | | | VSSASTKGPSVFPLAPSSKSTSGGTAALGCL |
| | | | VKDYFPEPVTVSWNSGALTSGVHTFPAVLQ |
| | | | SSGLYSLSSVVTVPSSSLGTQTYICNVNHKP |
| | | | SNTKVDKKVEPKSCDKTHTCPPCPAPPAAG |
| | | | PSVFLFPPKPKDTLMISRTPEVTCVVVDVSH |
| | | | EDPEVKFNWYVDGVEVHNAKTKPREEQY |
| | | | NSTYRVVSVLTVLHQDWLNGKEYKCKVSN |
| | | | KALAAPIEKTISKAKGQPREPQVYTLPPSRE |
| | | | EMTKNQVSLTCLVKGFYPSDIAVEWESNGQ |
| | | | PENNYKTTPPVLDSDGSFFLYSKLTVDKSR |
| | | | WQQGNVFSCSVMHEALHNHYTQKSLSLSP |
| | | | GKGGGGSEVQLLESGGGLVQPGGSLRLSCA |
| | | | ASGFMLSAEDVGWVRQAPGKGLEWVSAIL |
| | | | DFGGSTYYADSVKGRFTISRDNSKNTLYLQ |
| | | | MNSLRAEDTAVYYCARVVYHAGGGVTFD |
| | | | YWGQGTLVTVSS |
| | A-BGA- | SEQ ID | CAGGTGCAGCTGGTGCAGAGCGGCGCGG |
| | 4712/CEA heavy | NO: 178 | AAGTGAAAAAACCGGGCGCGAGCGTGAA |
| | chain DNA | | AGTGAGCTGCAAAGCGAGCGGCTATATTT |
| | | | TTACCAGCTATTACCTGCATTGGGTGCGCC |
| | | | AGGCGCCGGGCCAGGGCCTGGAATGGAT |
| | | | TGGCTATATTAACCCGCAGACCGGCAAGA |
| | | | CCAGCTATGCCCAGAAATTTCAGGGCCGC |
| | | | GTGACCATGACCCGCGATACCAGCACCAG |
| | | | CACCGTGTATATGGAACTGAGCAGCCTGC |
| | | | GCAGCGAAGATACCGCGGTGTATTATTGC |
| | | | GCGCGCGAATATGGCAACTATAACTATCCG |
| | | | CTGGATTATTGGGGCCAGGGCACCCTGGT |
| | | | GACCGTGAGCAGCGCTAGCACCAAGGGG |
| | | | CCCTCGGTCTTCCCCCTGGCACCCTCCTC |
| | | | CAAGAGTACTTCTGGGGGCACAGCGGCC |
| | | | CTGGGCTGCCTGGTCAAGGACTACTTCCC |
| | | | CGAACCGGTGACGGTGTCGTGGAACTCA |
| | | | GGCGCCCTGACCAGCGGCGTGCACACCTT |
| | | | CCCGGCTGTCCTACAGTCCTCAGGACTCT |
| | | | ACTCCCTCAGCAGCGTGGTGACCGTGCCC |
| | | | TCCAGCAGCTTGGGCACCCAGACCTACAT |
| | | | CTGCAACGTGAATCACAAGCCCAGCAAC |
| | | | ACCAAGGTGGACAAGAAAGTTGAGCCAA |
| | | | AGTCCTGTGACAAGACCCACACATGCCCC |
| | | | CCTTGTCCTGCTCCACCAGCTGCAGGACC |
| | | | AAGCGTGTTCCTGTTTCCACCCAAGCCCA |
| | | | AGGATACCCTGATGATCTCTCGGACCCCA |
| | | | GAGGTGACATGCGTGGTGGTGGATGTGAG |
| | | | CCACGAGGACCCCGAGGTGAAGTTCAAC |
| | | | TGGTATGTGGACGGCGTGGAGGTGCACAA |
| | | | TGCTAAGACCAAGCCCAGGGAGGAGCAG |
| | | | TACAACTCCACCTATAGAGTGGTGTCTGT |
| | | | GCTGACAGTGCTGCACCAGGATTGGCTGA |
| | | | ACGGCAAGGAGTATAAGTGCAAGGTGTCC |
| | | | AATAAGGCCCTGGCCGCTCCTATCGAGAA |
| | | | GACCATCTCTAAGGCCAAGGGCCAGCCCA |
| | | | GAGAGCCTCAGGTGTACACACTGCCTCCA |
| | | | TCCCGGGAAGAGATGACCAAGAACCAGG |
| | | | TGTCTCTGACATGTCTGGTCAAGGGCTTC |
| | | | TATCCCTCTGACATCGCCGTGGAGTGGGA |
| | | | GAGCAATGGCCAGCCTGAGAACAATTACA |
| | | | AGACCACACCCCCTGTGCTGGATTCCGAC |
| | | | GGCTCTTTCTTTCTGTATAGCAAGCTGACC |
| | | | GTGGACAAGTCCCGGTGGCAGCAGGGCA |
| | | | ACGTGTTCAGCTGTTCCGTGATGCACGAA |
| | | | GCTCTGCATAATCACTATACTCAGAAATCC |
| | | | CTGTCACTGTCACCTGGTAAAGGTGGAGG |
| | | | CGGTTCAGAGGTCCAGTTACTTGAGAGTG |
| | | | GTGGAGGTCTGGTCCAACCAGGAGGTTC |
| | | | GCTGCGTTTATCCTGCGCCGCGTCTGGATT |
| | | | CATGTTGTCCGCCGAAGACGTGGGTTGGG |
| | | | TGCGTCAAGCGCCGGGGAAAGGACTGGA |
| | | | ATGGGTCTCCGCCATCTTGGATTTTGGTGG |
| | | | TTCGACATACTATGCGGACAGTGTCAAAG |
| | | | GGCGCTTTACGATCTCGCGCGATAACTCA |
| | | | AAAAATACTCTTTACCTTCAAATGAATAGC |
| | | | CTTCGTGCTGAGGACACTGCGGTGTACTA |
| | | | CTGCGCCCGCGTCGTCTACCATGCTGGTG |
| | | | GTGGCGTCACCTTTGATTACTGGGGACAG |
| | | | GGCACCTTAGTTACAGTCTCATCG |
| | anti-CEA Ab | SEQ ID | DIQMTQSPSSLSASVGDRVTITCRASENQYG |
| | light chain AA | NO: 179 | YLAWYQQKPGKVPKLLIYNYKNLVEGVPS |
| | | | RFSGSGSGTDFTLTISSLQPEDVATYYCQHH |
| | | | LGTPYTFGQGTKVEIKRTVAAPSVFIFPPSDE |
| | | | QLKSGTASVVCLLNNFYPREAKVQWKVDN |
| | | | ALQSGNSQESVTEQDSKDSTYSLSSTLTLSK |
| | | | ADYEKHKVYACEVTHQGLSSPVTKSFNRG |
| | | | EC |
| | anti-CEA Ab light | SEQ ID | GATATTCAGATGACCCAGAGCCCGAGCAG |
| | chain DNA | NO: 180 | CCTGAGCGCGAGCGTGGGCGATCGCGTG |
| | | | ACCATTACCTGCCGCGCGAGCGAAAACCA |
| | | | GTATGGCTATCTGGCGTGGTATCAGCAGA |
| | | | AACCGGGCAAAGTGCCGAAACTGCTGAT |
| | | | TTATAACTATAAAAACCTGGTGGAAGGCG |
| | | | TGCCGAGCCGCTTTAGCGGCAGCGGCAGC |
| | | | GGCACCGATTTTACCCTGACCATTAGCAG |
| | | | CCTGCAGCCGGAAGATGTGGCGACCTATT |
| | | | ATTGCCAGCATCATCTGGGCACCCCGTATA |
| | | | CCTTTGGCCAGGGCACCAAAGTGGAAATT |
| | | | AAACGAACAGTGGCAGCCCCTTCCGTCTT |
| | | | CATTTTTCCCCCTTCTGACGAACAGCTGA |
| | | | AATCAGGAACTGCTAGCGTGGTCTGTCTG |
| | | | CTGAACAATTTCTACCCCAGAGAGGCCAA |
| | | | GGTGCAGTGGAAAGTCGATAACGCTCTGC |
| | | | AGTCCGGCAATTCTCAGGAGAGTGTGACC |
| | | | GAACAGGACTCAAAGGATAGCACATATTC |
| | | | CCTGTCTAGTACTCTGACCCTGAGCAAAG |
| | | | CAGACTACGAGAAGCACAAAGTGTATGCC |
| | | | TGTGAAGTCACACACCAGGGGCTGAGTT |
| | | | CACCAGTCACCAAGAGTTTCAACAGAGG |
| | | | GGAATGC |
| B-CD137/CEA | anti-CEA Ab | SEQ ID | QVQLVQSGAEVKKPGASVKVSCKASGYIFT |
| | heavy chain AA | NO: 181 | SYYLHWVRQAPGQGLEWIGYINPQTGKTS |
| | | | YAQKFQGRVTMTRDTSTSTVYMELSSLRSE |
| | | | DTAVYYCAREYGNYNYPLDYWGQGTLVT |
| | | | VSSASTKGPSVFPLAPSSKSTSGGTAALGCL |
| | | | VKDYFPEPVTVSWNSGALTSGVHTFPAVLQ |
| | | | SSGLYSLSSVVTVPSSSLGTQTYICNVNHKP |
| | | | SNTKVDKKVEPKSCDKTHTCPPCPAPPAAG |
| | | | PSVFLFPPKPKDTLMISRTPEVTCVVVDVSH |
| | | | EDPEVKFNWYVDGVEVHNAKTKPREEQY |
| | | | NSTYRVVSVLTVLHQDWLNGKEYKCKVSN |
| | | | KALAAPIEKTISKAKGQPREPQVYTLPPSRE |
| | | | EMTKNQVSLTCLVKGFYPSDIAVEWESNGQ |
| | | | PENNYKTTPPVLDSDGSFFLYSKLTVDKSR |
| | | | WQQGNVFSCSVMHEALHNHYTQKSLSLSP |
| | | | GK |
| | anti-CEA Ab | SEQ ID | CAGGTGCAGCTGGTGCAGAGCGGCGCGG |
| | heavy chain | NO: 182 | AAGTGAAAAAACCGGGCGCGAGCGTGAA |
| | DNA | | AGTGAGCTGCAAAGCGAGCGGCTATATTT |
| | | | TTACCAGCTATTACCTGCATTGGGTGCGCC |
| | | | AGGCGCCGGGCCAGGGCCTGGAATGGAT |
| | | | TGGCTATATTAACCCGCAGACCGGCAAGA |
| | | | CCAGCTATGCCCAGAAATTTCAGGGCCGC |
| | | | GTGACCATGACCCGCGATACCAGCACCAG |
| | | | CACCGTGTATATGGAACTGAGCAGCCTGC |
| | | | GCAGCGAAGATACCGCGGTGTATTATTGC |
| | | | GCGCGCGAATATGGCAACTATAACTATCCG |
| | | | CTGGATTATTGGGGCCAGGGCACCCTGGT |
| | | | GACCGTGAGCAGCGCTAGCACCAAGGGG |
| | | | CCCTCGGTCTTCCCCCTGGCACCCTCCTC |
| | | | CAAGAGTACTTCTGGGGGCACAGCGGCC |
| | | | CTGGGCTGCCTGGTCAAGGACTACTTCCC |
| | | | CGAACCGGTGACGGTGTCGTGGAACTCA |
| | | | GGCGCCCTGACCAGCGGCGTGCACACCTT |
| | | | CCCGGCTGTCCTACAGTCCTCAGGACTCT |
| | | | ACTCCCTCAGCAGCGTGGTGACCGTGCCC |
| | | | TCCAGCAGCTTGGGCACCCAGACCTACAT |
| | | | CTGCAACGTGAATCACAAGCCCAGCAAC |
| | | | ACCAAGGTGGACAAGAAAGTTGAGCCAA |
| | | | AGTCCTGTGACAAGACCCACACATGCCCC |
| | | | CCTTGTCCTGCTCCACCAGCTGCAGGACC |
| | | | AAGCGTGTTCCTGTTTCCACCCAAGCCCA |
| | | | AGGATACCCTGATGATCTCTCGGACCCCA |
| | | | GAGGTGACATGCGTGGTGGTGGATGTGAG |
| | | | CCACGAGGACCCCGAGGTGAAGTTCAAC |
| | | | TGGTATGTGGACGGCGTGGAGGTGCACAA |
| | | | TGCTAAGACCAAGCCCAGGGAGGAGCAG |
| | | | TACAACTCCACCTATAGAGTGGTGTCTGT |
| | | | GCTGACAGTGCTGCACCAGGATTGGCTGA |
| | | | ACGGCAAGGAGTATAAGTGCAAGGTGTCC |
| | | | AATAAGGCCCTGGCCGCTCCTATCGAGAA |
| | | | GACCATCTCTAAGGCCAAGGGCCAGCCCA |
| | | | GAGAGCCTCAGGTGTACACACTGCCTCCA |
| | | | TCCCGGGAAGAGATGACCAAGAACCAGG |
| | | | TGTCTCTGACATGTCTGGTCAAGGGCTTC |
| | | | TATCCCTCTGACATCGCCGTGGAGTGGGA |
| | | | GAGCAATGGCCAGCCTGAGAACAATTACA |
| | | | AGACCACACCCCCTGTGCTGGATTCCGAC |
| | | | GGCTCTTTCTTTCTGTATAGCAAGCTGACC |
| | | | GTGGACAAGTCCCGGTGGCAGCAGGGCA |
| | | | ACGTGTTCAGCTGTTCCGTGATGCACGAA |
| | | | GCTCTGCATAATCACTATACTCAGAAATCC |
| | | | CTGTCACTGTCACCTGGTAAA |
| | B-BGA- | SEQ ID | DIQMTQSPSSLSASVGDRVTITCRASENQYG |
| | 4712/CEA light | NO: 183 | YLAWYQQKPGKVPKLLIYNYKNLVEGVPS |
| | chain AA | | RFSGSGSGTDFTLTISSLQPEDVATYYCQHH |
| | | | LGTPYTFGQGTKVEIKRTVAAPSVFIFPPSDE |
| | | | QLKSGTASVVCLLNNFYPREAKVQWKVDN |
| | | | ALQSGNSQESVTEQDSKDSTYSLSSTLTLSK |
| | | | ADYEKHKVYACEVTHQGLSSPVTKSFNRG |
| | | | ECGGGGSEVQLLESGGGLVQPGGSLRLSCA |
| | | | ASGFMLSAEDVGWVRQAPGKGLEWVSAIL |
| | | | DFGGSTYYADSVKGRFTISRDNSKNTLYLQ |
| | | | MNSLRAEDTAVYYCARVVYHAGGGVTFD |
| | | | YWGQGTLVTVSS |
| | B-BGA- | SEQ ID | GATATTCAGATGACCCAGAGCCCGAGCAG |
| | 4712/CEA light | NO: 184 | CCTGAGCGCGAGCGTGGGCGATCGCGTG |
| | chain DNA | | ACCATTACCTGCCGCGCGAGCGAAAACCA |
| | | | GTATGGCTATCTGGCGTGGTATCAGCAGA |
| | | | AACCGGGCAAAGTGCCGAAACTGCTGAT |
| | | | TTATAACTATAAAAACCTGGTGGAAGGCG |
| | | | TGCCGAGCCGCTTTAGCGGCAGCGGCAGC |
| | | | GGCACCGATTTTACCCTGACCATTAGCAG |
| | | | CCTGCAGCCGGAAGATGTGGCGACCTATT |
| | | | ATTGCCAGCATCATCTGGGCACCCCGTATA |
| | | | CCTTTGGCCAGGGCACCAAAGTGGAAATT |
| | | | AAACGAACAGTGGCAGCCCCTTCCGTCTT |
| | | | CATTTTTCCCCCTTCTGACGAACAGCTGA |
| | | | AATCAGGAACTGCTAGCGTGGTCTGTCTG |
| | | | CTGAACAATTTCTACCCCAGAGAGGCCAA |
| | | | GGTGCAGTGGAAAGTCGATAACGCTCTGC |
| | | | AGTCCGGCAATTCTCAGGAGAGTGTGACC |
| | | | GAACAGGACTCAAAGGATAGCACATATTC |
| | | | CCTGTCTAGTACTCTGACCCTGAGCAAAG |
| | | | CAGACTACGAGAAGCACAAAGTGTATGCC |
| | | | TGTGAAGTCACACACCAGGGGCTGAGTT |
| | | | CACCAGTCACCAAGAGTTTCAACAGAGG |
| | | | GGAATGCGGGGGAGGCGGGTCCGAGGTC |
| | | | CAGTTACTTGAGAGTGGTGGAGGTCTGGT |
| | | | CCAACCAGGAGGTTCGCTGCGTTTATCCT |
| | | | GCGCCGCGTCTGGATTCATGTTGTCCGCC |
| | | | GAAGACGTGGGTTGGGTGCGTCAAGCGC |
| | | | CGGGGAAAGGACTGGAATGGGTCTCCGC |
| | | | CATCTTGGATTTTGGTGGTTCGACATACTA |
| | | | TGCGGACAGTGTCAAAGGGCGCTTTACGA |
| | | | TCTCGCGCGATAACTCAAAAAATACTCTTT |
| | | | ACCTTCAAATGAATAGCCTTCGTGCTGAG |
| | | | GACACTGCGGTGTACTACTGCGCCCGCGT |
| | | | CGTCTACCATGCTGGTGGTGGCGTCACCT |
| | | | TTGATTACTGGGGACAGGGCACCTTAGTT |
| | | | ACAGTCTCATCG |
| C-CD137/CEA | C-BGA- | SEQ ID | QVQLVQSGAEVKKPGASVKVSCKASGYIFT |
| | 4712/CEA heavy | NO: 185 | SYYLHWVRQAPGQGLEWIGYINPQTGKTS |
| | chain AA | | YAQKFQGRVTMTRDTSTSTVYMELSSLRSE |
| | | | DTAVYYCAREYGNYNYPLDYWGQGTLVT |
| | | | VSSASTKGPSVFPLAPSSKSTSGGTAALGCL |
| | | | VKDYFPEPVTVSWNSGALTSGVHTFPAVLQ |
| | | | SSGLYSLSSVVTVPSSSLGTQTYICNVNHKP |
| | | | SNTKVDKKVEPKSCDGGGGSEVQLLESGG |
| | | | GLVQPGGSLRLSCAASGFMLSAEDVGWVR |
| | | | QAPGKGLEWVSAILDFGGSTYYADSVKGR |
| | | | FTISRDNSKNTLYLQMNSLRAEDTAVYYCA |
| | | | RVVYHAGGGVTFDYWGQGTLVTVSSSGGG |
| | | | GSEPKSCDKTHTCPPCPAPPAAGPSVFLFPP |
| | | | KPKDTLMISRTPEVTCVVVDVSHEDPEVKF |
| | | | NWYVDGVEVHNAKTKPREEQYNSTYRVV |
| | | | SVLTVLHQDWLNGKEYKCKVSNKALAAPI |
| | | | EKTISKAKGQPREPQVYTLPPSREEMTKNQ |
| | | | VSLTCLVKGFYPSDIAVEWESNGQPENNYK |
| | | | TTPPVLDSDGSFFLYSKLTVDKSRWQQGNV |
| | | | FSCSVMHEALHNHYTQKSLSLSPGK |
| | C-BGA- | SEQ ID | CAGGTGCAGCTGGTGCAGAGCGGCGCGG |
| | 4712/CEA heavy | NO: 186 | AAGTGAAAAAACCGGGCGCGAGCGTGAA |
| | chain DNA | | AGTGAGCTGCAAAGCGAGCGGCTATATTT |
| | | | TTACCAGCTATTACCTGCATTGGGTGCGCC |
| | | | AGGCGCCGGGCCAGGGCCTGGAATGGAT |
| | | | TGGCTATATTAACCCGCAGACCGGCAAGA |
| | | | CCAGCTATGCCCAGAAATTTCAGGGCCGC |
| | | | GTGACCATGACCCGCGATACCAGCACCAG |
| | | | CACCGTGTATATGGAACTGAGCAGCCTGC |
| | | | GCAGCGAAGATACCGCGGTGTATTATTGC |
| | | | GCGCGCGAATATGGCAACTATAACTATCCG |
| | | | CTGGATTATTGGGGCCAGGGCACCCTGGT |
| | | | GACCGTGAGCAGCGCTAGCACCAAGGGG |
| | | | CCCTCGGTCTTCCCCCTGGCACCCTCCTC |
| | | | CAAGAGTACTTCTGGGGGCACAGCGGCC |
| | | | CTGGGCTGCCTGGTCAAGGACTACTTCCC |
| | | | CGAACCGGTGACGGTGTCGTGGAACTCA |
| | | | GGCGCCCTGACCAGCGGCGTGCACACCTT |
| | | | CCCGGCTGTCCTACAGTCCTCAGGACTCT |
| | | | ACTCCCTCAGCAGCGTGGTGACCGTGCCC |
| | | | TCCAGCAGCTTGGGCACCCAGACCTACAT |
| | | | CTGCAACGTGAATCACAAGCCCAGCAAC |
| | | | ACCAAGGTGGACAAGAAAGTTGAGCCAA |
| | | | AGTCCTGTGACGGGGGAGGCGGGTCcGA |
| | | | GGTCCAGTTACTTGAGAGTGGTGGAGGTC |
| | | | TGGTCCAACCAGGAGGTTCGCTGCGTTTA |
| | | | TCCTGCGCCGCGTCTGGATTCATGTTGTCC |
| | | | GCCGAAGACGTGGGTTGGGTGCGTCAAG |
| | | | CGCCGGGGAAAGGACTGGAATGGGTCTC |
| | | | CGCCATCTTGGATTTTGGTGGTTCGACATA |
| | | | CTATGCGGACAGTGTCAAAGGGCGCTTTA |
| | | | CGATCTCGCGCGATAACTCAAAAAATACT |
| | | | CTTTACCTTCAAATGAATAGCCTTCGTGCT |
| | | | GAGGACACTGCGGTGTACTACTGCGCCCG |
| | | | CGTCGTCTACCATGCTGGTGGTGGCGTCA |
| | | | CCTTTGATTACTGGGGACAGGGCACCTTA |
| | | | GTTACAGTCTCATCGTCTGGTGGAGGCGG |
| | | | TTCAGAGCCCAAATCTTGTGACAAAACTC |
| | | | ACACATGCCCACCGTGCCCAGCACCTCCT |
| | | | GCTGCCGGACCGTCAGTCTTCCTCTTCCC |
| | | | CCCAAAACCCAAGGACACCCTCATGATCT |
| | | | CCCGGACCCCTGAGGTCACATGCGTGGTG |
| | | | GTGGACGTGAGCCACGAAGACCCTGAGG |
| | | | TCAAGTTCAACTGGTACGTGGACGGCGTG |
| | | | GAGGTGCATAATGCCAAGACAAAGCCGC |
| | | | GGGAGGAGCAGTACAACAGCACGTACCG |
| | | | TGTGGTCAGCGTCCTCACCGTCCTGCACC |
| | | | AGGACTGGCTGAATGGCAAGGAGTACAA |
| | | | GTGCAAGGTCTCCAACAAAGCCCTCGCA |
| | | | GCCCCCATCGAGAAAACCATCTCCAAAGC |
| | | | CAAAGGGCAGCCCCGAGAACCACAGGTC |
| | | | TACACCCTGCCCCCATCCCGGGAGGAGAT |
| | | | GACCAAGAACCAGGTCAGCCTGACCTGC |
| | | | CTGGTCAAAGGCTTCTATCCCAGCGACAT |
| | | | CGCCGTGGAGTGGGAGAGCAATGGGCAG |
| | | | CCGGAGAACAACTACAAGACCACGCCTC |
| | | | CCGTGCTGGACTCCGACGGCTCCTTCTTC |
| | | | CTCTACAGCAAGCTCACCGTGGACAAGA |
| | | | GCAGGTGGCAGCAGGGGAACGTCTTCTC |
| | | | ATGCTCCGTGATGCATGAGGCTCTGCACA |
| | | | ACCACTACACGCAGAAGAGCCTCTCCCTG |
| | | | TCTCCGGGTAAA |
| | anti-CEA Ab | SEQ ID | DIQMTQSPSSLSASVGDRVTITCRASENQYG |
| | light chain AA | NO: 179 | YLAWYQQKPGKVPKLLIYNYKNLVEGVPS |
| | | | RFSGSGSGTDFTLTISSLQPEDVATYYCQHH |
| | | | LGTPYTFGQGTKVEIKRTVAAPSVFIFPPSDE |
| | | | QLKSGTASVVCLLNNFYPREAKVQWKVDN |
| | | | ALQSGNSQESVTEQDSKDSTYSLSSTLTLSK |
| | | | ADYEKHKVYACEVTHQGLSSPVTKSFNRG |
| | | | EC |
| | anti-CEA Ab | SEQ ID | GATATTCAGATGACCCAGAGCCCGAGCAG |
| | light chain DNA | NO: 180 | CCTGAGCGCGAGCGTGGGCGATCGCGTG |
| | | | ACCATTACCTGCCGCGCGAGCGAAAACCA |
| | | | GTATGGCTATCTGGCGTGGTATCAGCAGA |
| | | | AACCGGGCAAAGTGCCGAAACTGCTGAT |
| | | | TTATAACTATAAAAACCTGGTGGAAGGCG |
| | | | TGCCGAGCCGCTTTAGCGGCAGCGGCAGC |
| | | | GGCACCGATTTTACCCTGACCATTAGCAG |
| | | | CCTGCAGCCGGAAGATGTGGCGACCTATT |
| | | | ATTGCCAGCATCATCTGGGCACCCCGTATA |
| | | | CCTTTGGCCAGGGCACCAAAGTGGAAATT |
| | | | AAACGAACAGTGGCAGCCCCTTCCGTCTT |
| | | | CATTTTTCCCCCTTCTGACGAACAGCTGA |
| | | | AATCAGGAACTGCTAGCGTGGTCTGTCTG |
| | | | CTGAACAATTTCTACCCCAGAGAGGCCAA |
| | | | GGTGCAGTGGAAAGTCGATAACGCTCTGC |
| | | | AGTCCGGCAATTCTCAGGAGAGTGTGACC |
| | | | GAACAGGACTCAAAGGATAGCACATATTC |
| | | | CCTGTCTAGTACTCTGACCCTGAGCAAAG |
| | | | CAGACTACGAGAAGCACAAAGTGTATGCC |
| | | | TGTGAAGTCACACACCAGGGGCTGAGTT |
| | | | CACCAGTCACCAAGAGTTTCAACAGAGG |
| | | | GGAATGC |
| D-CD137/CEA | D-BGA- | SEQ ID | EVQLLESGGGLVQPGGSLRLSCAASGFMLS |
| | 4712/CEA heavy | NO: 187 | AEDVGWVRQAPGKGLEWVSAILDFGGSTY |
| | chain AA | | YADSVKGRFTISRDNSKNTLYLQMNSLRAE |
| | | | DTAVYYCARVVYHAGGGVTFDYWGQGTL |
| | | | VTVSSSGGGGSQVQLVQSGAEVKKPGASV |
| | | | KVSCKASGYIFTSYYLHWVRQAPGQGLEW |
| | | | IGYINPQTGKTSYAQKFQGRVTMTRDTSTST |
| | | | VYMELSSLRSEDTAVYYCAREYGNYNYPL |
| | | | DYWGQGTLVTVSSASTKGPSVFPLAPSSKS |
| | | | TSGGTAALGCLVKDYFPEPVTVSWNSGALT |
| | | | SGVHTFPAVLQSSGLYSLSSVVTVPSSSLGT |
| | | | QTYICNVNHKPSNTKVDKKVEPKSCDKTH |
| | | | TCPPCPAPPAAGPSVFLFPPKPKDTLMISRTP |
| | | | EVTCVVVDVSHEDPEVKFNWYVDGVEVH |
| | | | NAKTKPREEQYNSTYRVVSVLTVLHQDWL |
| | | | NGKEYKCKVSNKALAAPIEKTISKAKGQPR |
| | | | EPQVYTLPPSREEMTKNQVSLTCLVKGFYP |
| | | | SDIAVEWESNGQPENNYKTTPPVLDSDGSF |
| | | | FLYSKLTVDKSRWQQGNVFSCSVMHEALH |
| | | | NHYTQKSLSLSPGK |
| | D-BGA- | SEQ ID | GAGGTCCAGTTACTTGAGAGTGGTGGAG |
| | 4712/CEA heavy | NO: 188 | GTCTGGTCCAACCAGGAGGTTCGCTGCGT |
| | chain DNA | | TTATCCTGCGCCGCGTCTGGATTCATGTTG |
| | | | TCCGCCGAAGACGTGGGTTGGGTGCGTC |
| | | | AAGCGCCGGGGAAAGGACTGGAATGGGT |
| | | | CTCCGCCATCTTGGATTTTGGTGGTTCGAC |
| | | | ATACTATGCGGACAGTGTCAAAGGGCGCT |
| | | | TTACGATCTCGCGCGATAACTCAAAAAAT |
| | | | ACTCTTTACCTTCAAATGAATAGCCTTCGT |
| | | | GCTGAGGACACTGCGGTGTACTACTGCGC |
| | | | CCGCGTCGTCTACCATGCTGGTGGTGGCG |
| | | | TCACCTTTGATTACTGGGGACAGGGCACC |
| | | | TTAGTTACAGTCTCATCGTCCGGTGGAGG |
| | | | CGGTTCACAGGTGCAGCTGGTGCAGAGC |
| | | | GGCGCGGAAGTGAAAAAACCGGGCGCGA |
| | | | GCGTGAAAGTGAGCTGCAAAGCGAGCGG |
| | | | CTATATTTTTACCAGCTATTACCTGCATTGG |
| | | | GTGCGCCAGGCGCCGGGCCAGGGCCTGG |
| | | | AATGGATTGGCTATATTAACCCGCAGACCG |
| | | | GCAAGACCAGCTATGCCCAGAAATTTCAG |
| | | | GGCCGCGTGACCATGACCCGCGATACCAG |
| | | | CACCAGCACCGTGTATATGGAACTGAGCA |
| | | | GCCTGCGCAGCGAAGATACCGCGGTGTAT |
| | | | TATTGCGCGCGCGAATATGGCAACTATAAC |
| | | | TATCCGCTGGATTATTGGGGCCAGGGCAC |
| | | | CCTGGTGACCGTGAGCAGCGCTAGCACCA |
| | | | AGGGGCCCTCGGTCTTCCCCCTGGCACCC |
| | | | TCCTCCAAGAGTACTTCTGGGGGCACAGC |
| | | | GGCCCTGGGCTGCCTGGTCAAGGACTACT |
| | | | TCCCCGAACCGGTGACGGTGTCGTGGAA |
| | | | CTCAGGCGCCCTGACCAGCGGCGTGCAC |
| | | | ACCTTCCCGGCTGTCCTACAGTCCTCAGG |
| | | | ACTCTACTCCCTCAGCAGCGTGGTGACCG |
| | | | TGCCCTCCAGCAGCTTGGGCACCCAGACC |
| | | | TACATCTGCAACGTGAATCACAAGCCCAG |
| | | | CAACACCAAGGTGGACAAGAAAGTTGAG |
| | | | CCAAAGTCCTGTGACAAGACCCACACATG |
| | | | CCCCCCTTGTCCTGCTCCACCAGCTGCAG |
| | | | GACCAAGCGTGTTCCTGTTTCCACCCAAG |
| | | | CCCAAGGATACCCTGATGATCTCTCGGAC |
| | | | CCCAGAGGTGACATGCGTGGTGGTGGATG |
| | | | TGAGCCACGAGGACCCCGAGGTGAAGTT |
| | | | CAACTGGTATGTGGACGGCGTGGAGGTGC |
| | | | ACAATGCTAAGACCAAGCCCAGGGAGGA |
| | | | GCAGTACAACTCCACCTATAGAGTGGTGT |
| | | | CTGTGCTGACAGTGCTGCACCAGGATTGG |
| | | | CTGAACGGCAAGGAGTATAAGTGCAAGG |
| | | | TGTCCAATAAGGCCCTGGCCGCTCCTATC |
| | | | GAGAAGACCATCTCTAAGGCCAAGGGCC |
| | | | AGCCCAGAGAGCCTCAGGTGTACACACT |
| | | | GCCTCCATCCCGGGAAGAGATGACCAAG |
| | | | AACCAGGTGTCTCTGACATGTCTGGTCAA |
| | | | GGGCTTCTATCCCTCTGACATCGCCGTGG |
| | | | AGTGGGAGAGCAATGGCCAGCCTGAGAA |
| | | | CAATTACAAGACCACACCCCCTGTGCTGG |
| | | | ATTCCGACGGCTCTTTCTTTCTGTATAGCA |
| | | | AGCTGACCGTGGACAAGTCCCGGTGGCA |
| | | | GCAGGGCAACGTGTTCAGCTGTTCCGTGA |
| | | | TGCACGAAGCTCTGCATAATCACTATACTC |
| | | | AGAAATCCCTGTCACTGTCACCTGGTAAA |
| | anti-CEA Ab | SEQ ID | DIQMTQSPSSLSASVGDRVTITCRASENQYG |
| | light chain AA | NO: 179 | YLAWYQQKPGKVPKLLIYNYKNLVEGVPS |
| | | | RFSGSGSGTDFTLTISSLQPEDVATYYCQHH |
| | | | LGTPYTFGQGTKVEIKRTVAAPSVFIFPPSDE |
| | | | QLKSGTASVVCLLNNFYPREAKVQWKVDN |
| | | | ALQSGNSQESVTEQDSKDSTYSLSSTLTLSK |
| | | | ADYEKHKVYACEVTHQGLSSPVTKSFNRG |
| | | | EC |
| | anti-CEA Ab | SEQ ID | GATATTCAGATGACCCAGAGCCCGAGCAG |
| | light chain DNA | NO: 180 | CCTGAGCGCGAGCGTGGGCGATCGCGTG |
| | | | ACCATTACCTGCCGCGCGAGCGAAAACCA |
| | | | GTATGGCTATCTGGCGTGGTATCAGCAGA |
| | | | AACCGGGCAAAGTGCCGAAACTGCTGAT |
| | | | TTATAACTATAAAAACCTGGTGGAAGGCG |
| | | | TGCCGAGCCGCTTTAGCGGCAGCGGCAGC |
| | | | GGCACCGATTTTACCCTGACCATTAGCAG |
| | | | CCTGCAGCCGGAAGATGTGGCGACCTATT |
| | | | ATTGCCAGCATCATCTGGGCACCCCGTATA |
| | | | CCTTTGGCCAGGGCACCAAAGTGGAAATT |
| | | | AAACGAACAGTGGCAGCCCCTTCCGTCTT |
| | | | CATTTTTCCCCCTTCTGACGAACAGCTGA |
| | | | AATCAGGAACTGCTAGCGTGGTCTGTCTG |
| | | | CTGAACAATTTCTACCCCAGAGAGGCCAA |
| | | | GGTGCAGTGGAAAGTCGATAACGCTCTGC |
| | | | AGTCCGGCAATTCTCAGGAGAGTGTGACC |
| | | | GAACAGGACTCAAAGGATAGCACATATTC |
| | | | CCTGTCTAGTACTCTGACCCTGAGCAAAG |
| | | | CAGACTACGAGAAGCACAAAGTGTATGCC |
| | | | TGTGAAGTCACACACCAGGGGCTGAGTT |
| | | | CACCAGTCACCAAGAGTTTCAACAGAGG |
| | | | GGAATGC |

TABLE 21
Summary of yields and biochemical properties

| | | | | | human | | |
|---|---|---|---|---|---|---|---|
| | | | | | CD137 | | |
| | | | | | ECD- | | Hut78/ |
| | | | | | mIgG2a | CT26/CEA | huCD137 |
| Antibody | Yield | HMW | Monomer | LMW | SPR KD | EC50 | EC50 |
| formats | (mg/L) | (%) | (%) | (%) | (M) | (ug/ml) | (ug/ml) |
| A-CEAxCD137 | 30.00 | 1.28 | 96.34 | 2.39 | 3.60E−07 | 0.75 | 0.57 |
| D-CEAxCD137 | 11.00 | 1.80 | 98.19% | 0.0 | 7.80E−08 | 3.78 | 2.28 |

### Example 20. CD137 Based Multispecific Antibody A-CEA×CD137 Activates CD137 in a CEA Dependent Manner CD137 based multispecific antibodies induce CD137 activation in CD137 expressing cells

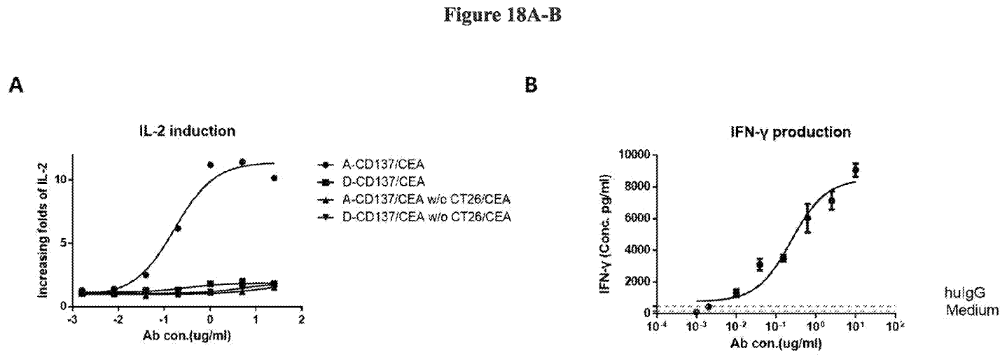

To test the ability of CD137 based multispecific antibodies to induce the response of CD137+ cells to the stimuli by CEA⁺ tumor cells, the Hut78/huCD137 were used to test for CD137 activation. CEA expressing CT26 (CT26/CEA) cells were generated by retroviral transduction into CT26 (ATCC CRL-2638) according to the protocols described previously (Zhang et al., 2005 supra). In OKT3 pre-coated 96-well plates, Hut78/huCD137 cells were co-cultured with CT26/CEA or CT26 (CEA-negative) cells overnight in the presence of CEA×CD137 multispecific constructs and interleukin-2 (IL-2) was measured as an indicator of CD137 activation in Hut78/huCD137 cells. As shown in FIG. 18A, A-CEA×CD137 induced Hut78/huCD137 cells to secrete IL-2 in a dose-dependent manner in the presence of the CEA⁺ CT26/CEA cells. Induction of IL-2 was not seen in the absence of CEA⁺ CT26/CEA cells.

### CD137 Based Multispecific Antibodies Induce CD137 Activation in Human Peripheral Blood Mononuclear Cells (PBMCs)

Human peripheral blood mononuclear cells (PBMCs) were isolated from whole blood of healthy donors by Ficoll (Histopaque-1077, Sigma-St. Louis MO) separation. OS8 expressing HEK293 (HEK293/OS8) cells was generated by retroviral transduction into HEK293 (ATCC CRL-1573) according to the protocols described previously (Zhang et al., 2005 supra). To determine whether CD137/CEA multispecific antibodies could activate T cells in the presence of CEA⁺ tumor cells, PBMCs (2×10⁵/well) were co-cultured with HEK293/OS8 and CT26/CEA cells in the presence of CD137/CEA multispecific antibodies for 48 hours. Activation of CD137 by CD137/CEA multispecific antibodies was determined by measuring IFN-γ in PBMCs. The results showed that A-CD137/CEA could induce significant CD137 activation in PBMCs (FIG. 18B) in the presence of CEA expressing cells.

### Example 21. Engineering and Affinity Maturation

### Engineering

Engineering was performed for the selected clone BGA-4712 to improve biochemical and biophysical properties. The considerations include amino acid compositions, heat stability (Tm), surface hydrophobicity, removal of post-translational modification (PTM) sites and isoelectronic points (pIs) while maintaining functional activities. Substitutions were made mainly in HCDRs and framework regions based on the BGA-4712 sequence. The substitutions included amino acid changes F28R, M29T, V35M, V37F or Y, G44E, L45R or G or Y, and W47G or S or F or L or R or Y, D62E, S75A, N84S, W103R (Kabat definition). The variants were expressed in both Fc fusion VH and A-CD137/CEA multispecific antibody format as described previously. The substitutions without significant affinity reduction were identified (Table 22). Combination of mutations were made. The sequences of BGA-4712-M3 and BGA-7556 are disclosed in Table 23 and 24.

### Affinity Maturation

To further explore potential effective CD137 based mechanisms of action (MOAs), we aimed to generate affinity matured BGA-4712-M3 variants with improved drug-developability by phage display. The library construction was described as before. In brief, a phagemid vector pCANTAB 5E (GE Healthcare) was used by standard molecular biology techniques to construct a phagemid designed to display CH3-G4S (linker)-BGA-4712-M3 (Table 25) on the surface of M13 bacteriophage as a fusion with the N-terminus of a fragment of the gene-3 minor coat protein. Generation of affinity-matured BGA-4712 variants was carried out by phage display using standard protocols (Silacci et al., (2005) Proteomics, 5, 2340-50; Zhao et al., (2014) PLoS One, 9, e111339). The phagemid was used as the template to construct phage-displayed libraries containing 2.0×10⁸ unique members. All three CDRs were randomized but each CDR had a maximum of one mutation in each clone except HCDR3, which could have two simultaneous mutations. Each position was randomized with an NNK codon (IUPAC code) encoding any amino acid or an amber stop codon.

The frequency of mutations in each HCDR after four rounds of selection was relatively high. FIG. 19 shows the sequences of HCDR regions after four rounds of selections. All mutations were introduced in BGA-7556 (SEQ ID NO: 86) to make affinity-matured variants except for BGA-3386, of which the mutations were introduced in BGA-4712-M3 (SEQ ID NO: 75). All variants were expressed as both monoclonal antibodies (VH-Fc) and their corresponding multispecific antibodies in Format A (A-CEA×CD137). The purified antibodies were concentrated to 0.5-10 mg/mL in PBS and stored in aliquots in a −80° C. freezer.

Affinity comparison on CD137 affinity matured variants was made by SPR assays using BIAcore™ T-200 (GE Life Sciences) and flow cytometry as described. The sequence information is shown in Table 28 and the results of SPR-determined binding profiles of anti-huCD137 antibodies were summarized in Table 26 and 27.

TABLE 22
Comparison of CD137 binding affinities

| | | | | EC50 on Hut78/ |
|---|---|---|---|---|
| Antibody | Format | SEC-HPLC (%) | KD (M) | huCD137 cells (ug/ml) |
| BGA-4712-M3 | Fc fusion VH | 97.98 | 1.69E−07 | 2.37 |
| BGA-4712-M3 | Format A-CEAxCD137 | 97.06 | 2.55E−07 | 0.52 |
| BGA-7556 | Fc fusion VH | 98.36 | 2.00E−07 | 5.71 |
| BGA-7556 | Format A-CEAxCD137 | 92.30 | 6.69E−07 | 3.23 |

TABLE 23
Sequence information of BGA-4712-M3

| Antibody | SEQ ID NO | | SEQUENCE |
|---|---|---|---|
| BGA-4712-M3 | SEQ ID NO: 75 | VH | EVQLLESGGGLVQPGGSLRLSCAASGF |
| | | | TLSAEDVGWVRQAPGKGLEWVSAILD |
| | | | FGGSTYYAESVKGRFTISRDNAKNTLY |
| | | | LQMSSLRAEDTAVYYCARVVYHAGGG |
| | | | VTFDYWGQGTLVTVSS |
| | SEQ ID NO: 76 | VH DNA | GAGGTCCAGTTACTTGAGAGTGGTGG |
| | | | AGGTCTGGTCCAACCAGGAGGTTCG |
| | | | CTGCGTTTATCCTGCGCCGCGTCTGG |
| | | | ATTCACGTTGTCCGCCGAAGACGTGG |
| | | | GTTGGGTGCGTCAAGCGCCGGGGAA |
| | | | AGGACTGGAATGGGTCTCCGCCATCT |
| | | | TGGATTTTGGTGGTTCGACATACTATG |
| | | | CGGAAAGTGTCAAAGGGCGCTTTAC |
| | | | GATCTCGCGCGATAACGCAAAAAATA |
| | | | CTCTTTACCTTCAAATGTCTAGCCTTC |
| | | | GTGCTGAGGACACTGCGGTGTACTAC |
| | | | TGCGCCCGCGTCGTCTACCATGCTGG |
| | | | TGGTGGCGTCACCTTTGATTACTGGG |
| | | | GACAGGGCACCTTAGTTACAGTCTCA |
| | | | TCG |
| BGA-4712-M3- | SEQ ID NO: 189 | AA | EVQLLESGGGLVQPGGSLRLSCAASGF |
| mutFc | | | TLSAEDVGWVRQAPGKGLEWVSAILD |
| | | | FGGSTYYAESVKGRFTISRDNAKNTLY |
| | | | LQMSSLRAEDTAVYYCARVVYHAGGG |
| | | | VTFDYWGQGTLVTVSSGGGGSDKTHT |
| | | | CPPCPAPPAAGPSVFLFPPKPKDTLMIS |
| | | | RTPEVTCVVVDVSHEDPEVKFNWYVD |
| | | | GVEVHNAKTKPREEQYNSTYRVVSVL |
| | | | TVLHQDWLNGKEYKCKVSNKALAAPI |
| | | | EKTISKAKGQPREPQVYTLPPSREEMT |
| | | | KNQVSLTCLVKGFYPSDIAVEWESNGQ |
| | | | PENNYKTTPPVLDSDGSFFLYSKLTVD |
| | | | KSRWQQGNVFSCSVMHEALHNHYTQ |
| | | | KSLSLSPGK |
| | SEQ ID NO: 190 | DNA | GAGGTCCAGTTACTTGAGAGTGGTGG |
| | | | AGGTCTGGTCCAACCAGGAGGTTCG |
| | | | CTGCGTTTATCCTGCGCCGCGTCTGG |
| | | | ATTCACGTTGTCCGCCGAAGACGTGG |
| | | | GTTGGGTGCGTCAAGCGCCGGGGAA |
| | | | AGGACTGGAATGGGTCTCCGCCATCT |
| | | | TGGATTTTGGTGGTTCGACATACTATG |
| | | | CGGAAAGTGTCAAAGGGCGCTTTAC |
| | | | GATCTCGCGCGATAACGCAAAAAATA |
| | | | CTCTTTACCTTCAAATGTCTAGCCTTC |
| | | | GTGCTGAGGACACTGCGGTGTACTAC |
| | | | TGCGCCCGCGTCGTCTACCATGCTGG |
| | | | TGGTGGCGTCACCTTTGATTACTGGG |
| | | | GACAGGGCACCTTAGTTACAGTCTCA |
| | | | TCGGGCGGCGGAGGGTCTGACAAGA |
| | | | CCCACACATGCCCCCCTTGTCCTGCT |
| | | | CCACCAGCTGCAGGACCAAGCGTGT |
| | | | TCCTGTTTCCACCCAAGCCCAAGGAT |
| | | | ACCCTGATGATCTCTCGGACCCCAGA |
| | | | GGTGACATGCGTGGTGGTGGATGTGA |
| | | | GCCACGAGGACCCCGAGGTGAAGTT |
| | | | CAACTGGTATGTGGACGGCGTGGAGG |
| | | | TGCACAATGCTAAGACCAAGCCCAGG |
| | | | GAGGAGCAGTACAACTCCACCTATAG |
| | | | AGTGGTGTCTGTGCTGACAGTGCTGC |
| | | | ACCAGGATTGGCTGAACGGCAAGGA |
| | | | GTATAAGTGCAAGGTGTCCAATAAGG |
| | | | CCCTGGCCGCTCCTATCGAGAAGACC |
| | | | ATCTCTAAGGCCAAGGGCCAGCCCAG |
| | | | AGAGCCTCAGGTGTACACACTGCCTC |
| | | | CATCCCGGGAAGAGATGACCAAGAA |
| | | | CCAGGTGTCTCTGACATGTCTGGTCA |
| | | | AGGGCTTCTATCCCTCTGACATCGCC |
| | | | GTGGAGTGGGAGAGCAATGGCCAGC |
| | | | CTGAGAACAATTACAAGACCACACCC |
| | | | CCTGTGCTGGATTCCGACGGCTCTTT |
| | | | CTTTCTGTATAGCAAGCTGACCGTGG |
| | | | ACAAGTCCCGGTGGCAGCAGGGCAA |
| | | | CGTGTTCAGCTGTTCCGTGATGCACG |
| | | | AAGCTCTGCATAATCACTATACTCAGA |
| | | | AATCCCTGTCACTGTCACCTGGTAAA |
| A-CEAxCD137- | SEQ ID NO: 191 | AA | GSATMDMRVPAQLLGLLLLWFPGSRSQ |
| M3 | | | VQLVQSGAEVKKPGASVKVSCKASGY |
| | | | IFTSYYLHWVRQAPGQGLEWIGYINPQ |
| | | | TGKTSYAQKFQGRVTMTRDTSTSTVY |
| | | | MELSSLRSEDTAVYYCAREYGNYNYP |
| | | | LDYWGQGTLVTVSSASTKGPSVFPLAP |
| | | | SSKSTSGGTAALGCLVKDYFPEPVTVS |
| | | | WNSGALTSGVHTFPAVLQSSGLYSLSS |
| | | | VVTVPSSSLGTQTYICNVNHKPSNTKV |
| | | | DKKVEPKSCDKTHTCPPCPAPPAAGPS |
| | | | VFLFPPKPKDTLMISRTPEVTCVVVDV |
| | | | SHEDPEVKFNWYVDGVEVHNAKTKP |
| | | | REEQYNSTYRVVSVLTVLHQDWLNGK |
| | | | EYKCKVSNKALAAPIEKTISKAKGQPR |
| | | | EPQVYTLPPSREEMTKNQVSLTCLVKG |
| | | | FYPSDIAVEWESNGQPENNYKTTPPVL |
| | | | DSDGSFFLYSKLTVDKSRWQQGNVFSC |
| | | | SVMHEALHNHYTQKSLSLSPGKGGGG |
| | | | SEVQLLESGGGLVQPGGSLRLSCAASG |
| | | | FTLSAEDVGWVRQAPGKGLEWVSAIL |
| | | | DFGGSTYYAESVKGRFTISRDNAKNTL |
| | | | YLQMSSLRAEDTAVYYCARVVYHAGG |
| | | | GVTFDYWGQGTLVTVSS |
| | SEQ ID NO: 192 | DNA | GGATCCGCCACCATGGATATGAGAGT |
| | | | TCCTGCTCAATTGCTGGGGTTGCTCTT |
| | | | GCTCTGGTTTCCTGGCTCGAGAAGCC |
| | | | AGGTGCAGCTGGTGCAGAGCGGCGC |
| | | | GGAAGTGAAAAAACCGGGCGCGAGC |
| | | | GTGAAAGTGAGCTGCAAAGCGAGCG |
| | | | GCTATATTTTTACCAGCTATTACCTGC |
| | | | ATTGGGTGCGCCAGGCGCCGGGCCA |
| | | | GGGCCTGGAATGGATTGGCTATATTAA |
| | | | CCCGCAGACCGGCAAGACCAGCTATG |
| | | | CCCAGAAATTTCAGGGCCGCGTGACC |
| | | | ATGACCCGCGATACCAGCACCAGCAC |
| | | | CGTGTATATGGAACTGAGCAGCCTGC |
| | | | GCAGCGAAGATACCGCGGTGTATTAT |
| | | | TGCGCGCGCGAATATGGCAACTATAA |
| | | | CTATCCGCTGGATTATTGGGGCCAGG |
| | | | GCACCCTGGTGACCGTGAGCAGCGC |
| | | | TAGCACCAAGGGGCCCTCGGTCTTCC |
| | | | CCCTGGCACCCTCCTCCAAGAGTACT |
| | | | TCTGGGGGCACAGCGGCCCTGGGCT |
| | | | GCCTGGTCAAGGACTACTTCCCCGAA |
| | | | CCGGTGACGGTGTCGTGGAACTCAG |
| | | | GCGCCCTGACCAGCGGCGTGCACAC |
| | | | CTTCCCGGCTGTCCTACAGTCCTCAG |
| | | | GACTCTACTCCCTCAGCAGCGTGGTG |
| | | | ACCGTGCCCTCCAGCAGCTTGGGCAC |
| | | | CCAGACCTACATCTGCAACGTGAATC |
| | | | ACAAGCCCAGCAACACCAAGGTGGA |
| | | | CAAGAAAGTTGAGCCAAAGTCCTGT |
| | | | GACAAGACCCACACATGCCCCCCTTG |
| | | | TCCTGCTCCACCAGCTGCAGGACCAA |
| | | | GCGTGTTCCTGTTTCCACCCAAGCCC |
| | | | AAGGATACCCTGATGATCTCTCGGAC |
| | | | CCCAGAGGTGACATGCGTGGTGGTG |
| | | | GATGTGAGCCACGAGGACCCCGAGG |
| | | | TGAAGTTCAACTGGTATGTGGACGGC |
| | | | GTGGAGGTGCACAATGCTAAGACCA |
| | | | AGCCCAGGGAGGAGCAGTACAACTC |
| | | | CACCTATAGAGTGGTGTCTGTGCTGA |
| | | | CAGTGCTGCACCAGGATTGGCTGAAC |
| | | | GGCAAGGAGTATAAGTGCAAGGTGTC |
| | | | CAATAAGGCCCTGGCCGCTCCTATCG |
| | | | AGAAGACCATCTCTAAGGCCAAGGG |
| | | | CCAGCCCAGAGAGCCTCAGGTGTAC |
| | | | ACACTGCCTCCATCCCGGGAAGAGAT |
| | | | GACCAAGAACCAGGTGTCTCTGACAT |
| | | | GTCTGGTCAAGGGCTTCTATCCCTCT |
| | | | GACATCGCCGTGGAGTGGGAGAGCA |
| | | | ATGGCCAGCCTGAGAACAATTACAAG |
| | | | ACCACACCCCCTGTGCTGGATTCCGA |
| | | | CGGCTCTTTCTTTCTGTATAGCAAGCT |
| | | | GACCGTGGACAAGTCCCGGTGGCAG |
| | | | CAGGGCAACGTGTTCAGCTGTTCCGT |
| | | | GATGCACGAAGCTCTGCATAATCACT |
| | | | ATACTCAGAAATCCCTGTCACTGTCA |
| | | | CCTGGTAAAGGTGGAGGCGGTTCAG |
| | | | AGGTCCAGTTACTTGAGAGTGGTGGA |
| | | | GGTCTGGTCCAACCAGGAGGTTCGCT |
| | | | GCGTTTATCCTGCGCCGCGTCTGGATT |
| | | | CACGTTGTCCGCCGAAGACGTGGGTT |
| | | | GGGTGCGTCAAGCGCCGGGGAAAGG |
| | | | ACTGGAATGGGTCTCCGCCATCTTGG |
| | | | ATTTTGGTGGTTCGACATACTATGCGG |
| | | | AAAGTGTCAAAGGGCGCTTTACGATC |
| | | | TCGCGCGATAACGCAAAAAATACTCT |
| | | | TTACCTTCAAATGTCTAGCCTTCGTGC |
| | | | TGAGGACACTGCGGTGTACTACTGCG |
| | | | CCCGCGTCGTCTACCATGCTGGTGGT |
| | | | GGCGTCACCTTTGATTACTGGGGACA |
| | | | GGGCACCTTAGTTACAGTCTCATCGT |
| | | | GAAAGCTTCGTCAG |

TABLE 24
Sequence information of BGA-7556 in Fc fusion VH and A-CEAxCD137
multispecific antibody format

| Antibody | SEQ ID NO | | SEQUENCE |
|---|---|---|---|
| BGA-7556 | SEQ ID NO: 86 | VH | EVQLLESGGGLVQPGGSLRLSCAASG |
| | | | FTLSAEDVGWVRQAPGKGLEWVSAI |
| | | | LDFGGSTYYAESVKGRFTISRDNAKN |
| | | | TLYLQMSSLRAEDTAVYYCARVVYH |
| | | | AGGGVTFDYRGQGTQVTVSS |
| | SEQ ID NO: 87 | VH DNA | GAGGTCCAGTTACTTGAGAGTGGT |
| | | | GGAGGTCTGGTCCAACCAGGAGGT |
| | | | TCGCTGCGTTTATCCTGCGCCGCGT |
| | | | CTGGATTCACGTTGTCCGCCGAAGA |
| | | | CGTGGGTTGGGTGCGTCAAGCGCC |
| | | | GGGGAAAGGACTGGAATGGGTCTC |
| | | | CGCCATCTTGGATTTTGGTGGTTCG |
| | | | ACATACTATGCGGAAAGTGTCAAAG |
| | | | GGCGCTTTACGATCTCGCGCGATAA |
| | | | CGCAAAAAATACTCTTTACCTTCAA |
| | | | ATGTCTAGCCTTCGTGCTGAGGACA |
| | | | CTGCGGTGTACTACTGCGCCCGCGT |
| | | | CGTCTACCATGCTGGTGGTGGCGTC |
| | | | ACCTTTGATTACCGGGGACAGGGCA |
| | | | CCCAAGTTACAGTCTCATCG |
| BGA-7556- | SEQ ID NO: 193 | AA | EVQLLESGGGLVQPGGSLRLSCAASG |
| mutFc | | | FTLSAEDVGWVRQAPGKGLEWVSAI |
| | | | LDFGGSTYYAESVKGRFTISRDNAKN |
| | | | TLYLQMSSLRAEDTAVYYCARVVYH |
| | | | AGGGVTFDYRGQGTQVTVSSGGGGS |
| | | | DKTHTCPPCPAPPAAGPSVFLFPPKPK |
| | | | DTLMISRTPEVTCVVVDVSHEDPEVK |
| | | | FNWYVDGVEVHNAKTKPREEQYNS |
| | | | TYRVVSVLTVLHQDWLNGKEYKCK |
| | | | VSNKALAAPIEKTISKAKGQPREPQV |
| | | | YTLPPSREEMTKNQVSLTCLVKGFYP |
| | | | SDIAVEWESNGQPENNYKTTPPVLDS |
| | | | DGSFFLYSKLTVDKSRWQQGNVFSCS |
| | | | VMHEALHNHYTQKSLSLSPGK |
| | SEQ ID NO: 194 | DNA | GAGGTCCAGTTACTTGAGAGTGGT |
| | | | GGAGGTCTGGTCCAACCAGGAGGT |
| | | | TCGCTGCGTTTATCCTGCGCCGCGT |
| | | | CTGGATTCACGTTGTCCGCCGAAGA |
| | | | CGTGGGTTGGGTGCGTCAAGCGCC |
| | | | GGGGAAAGGACTGGAATGGGTCTC |
| | | | CGCCATCTTGGATTTTGGTGGTTCG |
| | | | ACATACTATGCGGAAAGTGTCAAAG |
| | | | GGCGCTTTACGATCTCGCGCGATAA |
| | | | CGCAAAAAATACTCTTTACCTTCAA |
| | | | ATGTCTAGCCTTCGTGCTGAGGACA |
| | | | CTGCGGTGTACTACTGCGCCCGCGT |
| | | | CGTCTACCATGCTGGTGGTGGCGTC |
| | | | ACCTTTGATTACCGGGGACAGGGCA |
| | | | CCCAAGTTACAGTCTCATCGGGCGG |
| | | | CGGAGGGTCTGACAAGACCCACAC |
| | | | ATGCCCCCCTTGTCCTGCTCCACCA |
| | | | GCTGCAGGACCAAGCGTGTTCCTGT |
| | | | TTCCACCCAAGCCCAAGGATACCCT |
| | | | GATGATCTCTCGGACCCCAGAGGTG |
| | | | ACATGCGTGGTGGTGGATGTGAGCC |
| | | | ACGAGGACCCCGAGGTGAAGTTCA |
| | | | ACTGGTATGTGGACGGCGTGGAGG |
| | | | TGCACAATGCTAAGACCAAGCCCA |
| | | | GGGAGGAGCAGTACAACTCCACCT |
| | | | ATAGAGTGGTGTCTGTGCTGACAGT |
| | | | GCTGCACCAGGATTGGCTGAACGG |
| | | | CAAGGAGTATAAGTGCAAGGTGTCC |
| | | | AATAAGGCCCTGGCCGCTCCTATCG |
| | | | AGAAGACCATCTCTAAGGCCAAGG |
| | | | GCCAGCCCAGAGAGCCTCAGGTGT |
| | | | ACACACTGCCTCCATCCCGGGAAG |
| | | | AGATGACCAAGAACCAGGTGTCTC |
| | | | TGACATGTCTGGTCAAGGGCTTCTA |
| | | | TCCCTCTGACATCGCCGTGGAGTGG |
| | | | GAGAGCAATGGCCAGCCTGAGAAC |
| | | | AATTACAAGACCACACCCCCTGTGC |
| | | | TGGATTCCGACGGCTCTTTCTTTCT |
| | | | GTATAGCAAGCTGACCGTGGACAA |
| | | | GTCCCGGTGGCAGCAGGGCAACGT |
| | | | GTTCAGCTGTTCCGTGATGCACGAA |
| | | | GCTCTGCATAATCACTATACTCAGA |
| | | | AATCCCTGTCACTGTCACCTGGTAA |
| | | | A |
| Format A- | SEQ ID NO: 195 | AA | QVQLVQSGAEVKKPGASVKVSCKAS |
| BGA-7556 | | | GYIFTSYYLHWVRQAPGQGLEWIGY |
| | | | INPQTGKTSYAQKFQGRVTMTRDTST |
| | | | STVYMELSSLRSEDTAVYYCAREYG |
| | | | NYNYPLDYWGQGTLVTVSSASTKGP |
| | | | SVFPLAPSSKSTSGGTAALGCLVKDY |
| | | | FPEPVTVSWNSGALTSGVHTFPAVLQ |
| | | | SSGLYSLSSVVTVPSSSLGTQTYICNV |
| | | | NHKPSNTKVDKKVEPKSCDKTHTCP |
| | | | PCPAPPAAGPSVFLFPPKPKDTLMISR |
| | | | TPEVTCVVVDVSHEDPEVKFNWYVD |
| | | | GVEVHNAKTKPREEQYNSTYRVVSV |
| | | | LTVLHQDWLNGKEYKCKVSNKALA |
| | | | APIEKTISKAKGQPREPQVYTLPPSRE |
| | | | EMTKNQVSLTCLVKGFYPSDIAVEWE |
| | | | SNGQPENNYKTTPPVLDSDGSFFLYS |
| | | | KLTVDKSRWQQGNVFSCSVMHEAL |
| | | | HNHYTQKSLSLSPGKGGGGSEVQLL |
| | | | ESGGGLVQPGGSLRLSCAASGFTLSA |
| | | | EDVGWVRQAPGKGLEWVSAILDFG |
| | | | GSTYYAESVKGRFTISRDNAKNTLYL |
| | | | QMSSLRAEDTAVYYCARVVYHAGG |
| | | | GVTFDYRGQGTQVTVSS |
| | SEQ ID NO: 196 | DNA | CAGGTGCAGCTGGTGCAGAGCGGC |
| | | | GCGGAAGTGAAAAAACCGGGCGCG |
| | | | AGCGTGAAAGTGAGCTGCAAAGCG |
| | | | AGCGGCTATATTTTTACCAGCTATTA |
| | | | CCTGCATTGGGTGCGCCAGGCGCCG |
| | | | GGCCAGGGCCTGGAATGGATTGGCT |
| | | | ATATTAACCCGCAGACCGGCAAGAC |
| | | | CAGCTATGCCCAGAAATTTCAGGGC |
| | | | CGCGTGACCATGACCCGCGATACCA |
| | | | GCACCAGCACCGTGTATATGGAACT |
| | | | GAGCAGCCTGCGCAGCGAAGATAC |
| | | | CGCGGTGTATTATTGCGCGCGCGAA |
| | | | TATGGCAACTATAACTATCCGCTGG |
| | | | ATTATTGGGGCCAGGGCACCCTGGT |
| | | | GACCGTGAGCAGCGCTAGCACCAA |
| | | | GGGGCCCTCGGTCTTCCCCCTGGCA |
| | | | CCCTCCTCCAAGAGTACTTCTGGGG |
| | | | GCACAGCGGCCCTGGGCTGCCTGG |
| | | | TCAAGGACTACTTCCCCGAACCGGT |
| | | | GACGGTGTCGTGGAACTCAGGCGC |
| | | | CCTGACCAGCGGCGTGCACACCTTC |
| | | | CCGGCTGTCCTACAGTCCTCAGGAC |
| | | | TCTACTCCCTCAGCAGCGTGGTGAC |
| | | | CGTGCCCTCCAGCAGCTTGGGCACC |
| | | | CAGACCTACATCTGCAACGTGAATC |
| | | | ACAAGCCCAGCAACACCAAGGTGG |
| | | | ACAAGAAAGTTGAGCCAAAGTCCT |
| | | | GTGACAAGACCCACACATGCCCCC |
| | | | CTTGTCCTGCTCCACCAGCTGCAGG |
| | | | ACCAAGCGTGTTCCTGTTTCCACCC |
| | | | AAGCCCAAGGATACCCTGATGATCT |
| | | | CTCGGACCCCAGAGGTGACATGCG |
| | | | TGGTGGTGGATGTGAGCCACGAGG |
| | | | ACCCCGAGGTGAAGTTCAACTGGT |
| | | | ATGTGGACGGCGTGGAGGTGCACA |
| | | | ATGCTAAGACCAAGCCCAGGGAGG |
| | | | AGCAGTACAACTCCACCTATAGAGT |
| | | | GGTGTCTGTGCTGACAGTGCTGCAC |
| | | | CAGGATTGGCTGAACGGCAAGGAG |
| | | | TATAAGTGCAAGGTGTCCAATAAGG |
| | | | CCCTGGCCGCTCCTATCGAGAAGAC |
| | | | CATCTCTAAGGCCAAGGGCCAGCCC |
| | | | AGAGAGCCTCAGGTGTACACACTG |
| | | | CCTCCATCCCGGGAAGAGATGACCA |
| | | | AGAACCAGGTGTCTCTGACATGTCT |
| | | | GGTCAAGGGCTTCTATCCCTCTGAC |
| | | | ATCGCCGTGGAGTGGGAGAGCAAT |
| | | | GGCCAGCCTGAGAACAATTACAAG |
| | | | ACCACACCCCCTGTGCTGGATTCCG |
| | | | ACGGCTCTTTCTTTCTGTATAGCAA |
| | | | GCTGACCGTGGACAAGTCCCGGTG |
| | | | GCAGCAGGGCAACGTGTTCAGCTG |
| | | | TTCCGTGATGCACGAAGCTCTGCAT |
| | | | AATCACTATACTCAGAAATCCCTGT |
| | | | CACTGTCACCTGGTAAAGGTGGAG |
| | | | GCGGTTCAGAGGTCCAGTTACTTGA |
| | | | GAGTGGTGGAGGTCTGGTCCAACC |
| | | | AGGAGGTTCGCTGCGTTTATCCTGC |
| | | | GCCGCGTCTGGATTCACGTTGTCCG |
| | | | CCGAAGACGTGGGTTGGGTGCGTC |
| | | | AAGCGCCGGGGAAAGGACTGGAAT |
| | | | GGGTCTCCGCCATCTTGGATTTTGG |
| | | | TGGTTCGACATACTATGCGGAAAGT |
| | | | GTCAAAGGGCGCTTTACGATCTCGC |
| | | | GCGATAACGCAAAAAATACTCTTTA |
| | | | CCTTCAAATGTCTAGCCTTCGTGCT |
| | | | GAGGACACTGCGGTGTACTACTGCG |
| | | | CCCGCGTCGTCTACCATGCTGGTGG |
| | | | TGGCGTCACCTTTGATTACCGGGGA |
| | | | CAGGGCACCCAAGTTACAGTCTCAT |
| | | | CG |

TABLE 25
Sequence information

| CONSTRUCT | SEQ ID NO | | SEQUENCE |
|---|---|---|---|
| CH3 | SEQ ID NO: | AA | GQPREPQVYTLPPSREEMTKNQVSL |
| | 197 | | TCLVKGFYPSDIAVEWESNGQPENN |
| | | | YKTTPPVLDSDGSFFLYSKLTVDKSR |
| | | | WQQGNVFSCSVMHEALHNHYTQK |
| | | | SLSLSPGK |
| | SEQ ID NO: | DNA | GGCCAGCCCAGAGAGCCTCAGGT |
| | 198 | | GTACACACTGCCTCCATCCCGGGA |
| | | | AGAGATGACCAAGAACCAGGTGT |
| | | | CTCTGACATGTCTGGTCAAGGGCT |
| | | | TCTATCCCTCTGACATCGCCGTGGA |
| | | | GTGGGAGAGCAATGGCCAGCCTG |
| | | | AGAACAATTACAAGACCACACCCC |
| | | | CTGTGCTGGATTCCGACGGCTCTTT |
| | | | CTTTCTGTATAGCAAGCTGACCGTG |
| | | | GACAAGTCCCGGTGGCAGCAGGG |
| | | | CAACGTGTTCAGCTGTTCCGTGAT |
| | | | GCACGAAGCTCTGCATAATCACTAT |
| | | | ACTCAGAAATCCCTGTCACTGTCA |
| | | | CCTGGTAAA |
| CH3- BGA- | SEQ ID NO: | AA | GQPREPQVYTLPPSREEMTKNQVSL |
| 4712-M3 | 199 | | TCLVKGFYPSDIAVEWESNGQPENN |
| | | | YKTTPPVLDSDGSFFLYSKLTVDKSR |
| | | | WQQGNVFSCSVMHEALHNHYTQK |
| | | | SLSLSPGKGGGGSEVQLLESGGGLV |
| | | | QPGGSLRLSCAASGFTLSAEDVGW |
| | | | VRQAPGKGLEWVSAILDFGGSTYY |
| | | | AESVKGRFTISRDNAKNTLYLQMSS |
| | | | LRAEDTAVYYCARVVYHAGGGVTF |
| | | | DYWGQGTLVTVSS |
| | SEQ ID NO: | DNA | GGCCAGCCCAGAGAGCCTCAGGT |
| | 200 | | GTACACACTGCCTCCATCCCGGGA |
| | | | AGAGATGACCAAGAACCAGGTGT |
| | | | CTCTGACATGTCTGGTCAAGGGCT |
| | | | TCTATCCCTCTGACATCGCCGTGGA |
| | | | GTGGGAGAGCAATGGCCAGCCTG |
| | | | AGAACAATTACAAGACCACACCCC |
| | | | CTGTGCTGGATTCCGACGGCTCTTT |
| | | | CTTTCTGTATAGCAAGCTGACCGTG |
| | | | GACAAGTCCCGGTGGCAGCAGGG |
| | | | CAACGTGTTCAGCTGTTCCGTGAT |
| | | | GCACGAAGCTCTGCATAATCACTAT |
| | | | ACTCAGAAATCCCTGTCACTGTCA |
| | | | CCTGGTAAAGGTGGAGGCGGTTCA |
| | | | GAGGTCCAGTTACTTGAGAGTGGT |
| | | | GGAGGTCTGGTCCAACCAGGAGG |
| | | | TTCGCTGCGTTTATCCTGCGCCGCG |
| | | | TCTGGATTCACGTTGTCCGCCGAA |
| | | | GACGTGGGTTGGGTGCGTCAAGCG |
| | | | CCGGGGAAAGGACTGGAATGGGT |
| | | | CTCCGCCATCTTGGATTTTGGTGGT |
| | | | TCGACATACTATGCGGAAAGTGTC |
| | | | AAAGGGCGCTTTACGATCTCGCGC |
| | | | GATAACGCAAAAAATACTCTTTACC |
| | | | TTCAAATGTCTAGCCTTCGTGCTGA |
| | | | GGACACTGCGGTGTACTACTGCGC |
| | | | CCGCGTCGTCTACCATGCTGGTGG |
| | | | TGGCGTCACCTTTGATTACTGGGG |
| | | | ACAGGGCACCTTAGTTACAGTCTC |
| | | | ATCG |

TABLE 26
Affinity comparison of affinity matured
BGA-4712 variants as Fc fusion antibody

| Clone # | Format | SEC-HPLC (%) | KD (M) |
|---|---|---|---|
| BGA-5623 | Fc-fusion VH | 97.62 | 5.03E−08 |
| BGA-2690 | | 97.82 | 6.75E−05 |
| BGA-3849 | | 98.75 | 7.63E−08 |
| BGA-7916 | | 97.57 | 8.77E−08 |
| BGA-4988 | | 97.71 | 1.03E−06 |
| BGA-5164 | | 99.07 | 1.38E−05 |
| BGA-3953 | | 97.29 | 4.83E−08 |
| BGA-5385 | | 97.86 | 1.00E−07 |
| BGA-9468 | | 97.56 | 1.90E−07 |
| BGA-3285 | | 97.83 | 1.86E−07 |
| BGA-9442 | | 98.6 | 1.51E−07 |
| BGA-7746 | | 97.68 | 1.93E−07 |
| BGA-8425 | | 98.2 | 9.34E−08 |
| BGA-6468 | | 92.00 | 1.12E−08 |
| BGA-4712-M3 | | 97.98 | 1.69E−07 |
| BGA-7556 | | 98.36 | 2.00E−07 |

TABLE 27
Affinity Comparison of affinity matured BGA-4712 variants

| | | SEC- | |
|---|---|---|---|
| Clone # | Format | HPLC (%) | KD (M) |
| Format A -BGA-5623 | A-CEAxCD137 | 94.55 | 9.48E−08 |
| Format A - BGA-2690 | | — | — |
| Format A - BGA-3849 | | — | — |
| Format A- BGA-7916 | | 98.38 | 2.03E−07 |
| Format A - BGA-4988 | | 98.43 | — |
| Format A - BGA-5164 | | 98.36 | — |
| Format A - BGA-3953 | | 96.19 | 1.15E−07 |
| Format A - BGA-5385 | | 97.18 | 1.54E−07 |
| Format A - BGA-9468 | | 98.55 | 8.73E−07 |
| Format A - BGA-3285 | | 97.32 | 7.98E−07 |
| Format A - BGA-9442 | | 98.67 | 1.64E−07 |
| Format A - BGA-7746 | | 97.66 | 2.45E−07 |
| Format A - BGA-8425 | | 95.69 | 1.89E−07 |
| Format A - BGA-3386 | | 87.00 | 2.14E−08 |
| Format A - BGA-4712-M3 | | 97.06 | 2.89E−07 |
| Format A - BGA-7556 | | 92.3 | 6.49E−07 |

TABLE 28
Sequence information for affinity matured BGA-4712 variants

| | | |
|---|---|---|
| SEQ ID NO: | BGA-5623 | EVQLLESGGGLVQPGGSLRLSCAASGFTVSAEDVGWV |
| 84 | AA | RQAPGKGLEWVSAILDKGGSTYYAESVKGRFTISRDN |
| | | AKNTLYLQMSSLRAEDTAVYYCARIVYHAGGGVTFD |
| | | TRGQGTQVTVSS |
| SEQ ID NO: | BGA-5623 | GAGGTCCAGTTACTTGAGAGTGGTGGAGGTCTGGTC |
| 85 | DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATAAGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCATTGTCTACCATGCTGGTGGTGGCGTCAC |
| | | CTTTGATACTCGGGGACAGGGCACCCAAGTTACAGT |
| | | CTCATCG |
| SEQ ID NO: | BGA-2690 | EVQLLESGGGLVQPGGSLRLSCAASGFTLSREDVGWV |
| 201 | AA | RQAPGKGLEWVSAILDFGSSTYYAESVKGRFTISRDNA |
| | | KNTLYLQMSSLRAEDTAVYYCARVVYHAGGGVTYD |
| | | YRGQGTQVTVSS |
| SEQ ID NO: | BGA-2690 | GAGGTCCAGTTACTTGAGAGTGGTGGAGGTCTGGTC |
| 202 | DNA | CAACCAGGAGGTTCGCTGCGTTTATCCTGCGCCGCG |
| | | TCTGGATTCACGCTTTCCAGAGAAGACGTGGGTTGG |
| | | GTGCGTCAAGCGCCGGGGAAAGGACTGGAATGGGT |
| | | CTCCGCCATCTTGGATTTCGGTTCTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCGTTGTCTACCATGCTGGTGGTGGCGTCACC |
| | | TATGATTATCGGGGACAGGGCACCCAAGTTACAGTC |
| | | TCATCG |
| SEQ ID NO: | BGA-3849 | EVQLLESGGGLVQPGGSLRLSCAASGFTLSHEDVGWV |
| 203 | AA | RQAPGKGLEWVSAILDSGGSTYYAESVKGRFTISRDN |
| | | AKNTLYLQMSSLRAEDTAVYYCARYVYHAGGGVTFD |
| | | TRGQGTQVTVSS |
| SEQ ID NO: | BGA-3849 | GAGGTCCAGTTACTTGAGAGTGGTGGAGGTCTGGTC |
| 204 | DNA | CAACCAGGAGGTTCGCTGCGTTTATCCTGCGCCGCG |
| | | TCTGGATTCACGCTTTCCCATGAAGACGTGGGTTGG |
| | | GTGCGTCAAGCGCCGGGGAAAGGACTGGAATGGGT |
| | | CTCCGCCATCTTGGATTCCGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCTATGTCTACCATGCTGGTGGTGGCGTCACC |
| | | TTTGATACTCGGGGACAGGGCACCCAAGTTACAGTC |
| | | TCATCG |
| SEQ ID NO: | BGA-7916 | EVQLLESGGGLVQPGGSLRLSCAASGFTVSAEDVGWV |
| 205 | AA | RQAPGKGLEWVSAILDSGGSTYYAESVKGRFTISRDN |
| | | AKNTLYLQMSSLRAEDTAVYYCARVVYHAGGGVTY |
| | | DYRGQGTQVTVSS |
| SEQ ID NO: | BGA-7916 | GAGGTCCAGTTACTTGAGAGTGGTGGAGGTCTGGTC |
| 206 | DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATTCGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTGTCTACCATGCTGGTGGTGGCGTCAC |
| | | CTATGATACTCGGGGACAGGGCACCCAAGTTACAGT |
| | | CTCATCG |
| SEQ ID NO: | BGA-4988 | EVQLLESGGGLVQPGGSLRLSCAASGFTVSAEDVGWV |
| 207 | AA | RQAPGKGLEWVSAILDRGGSTYYAESVKGRFTISRDN |
| | | AKNTLYLQMSSLRAEDTAVYYCARVVYHAGGGVTY |
| | | DYRGQGTQVTVSS |
| SEQ ID NO: | BGA-4988 | GAGGTCCAGTTACTTGAGAGTGGTGGAGGTCTGGTC |
| 208 | DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATCGGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTGTCTACCATGCTGGTGGTGGCGTCAC |
| | | CTATGATACTCGGGGACAGGGCACCCAAGTTACAGT |
| | | CTCATCG |
| SEQ ID NO: | BGA-5164 | EVQLLESGGGLVQPGGSLRLSCAASGFTLSAEDVGWV |
| 209 | AA | RQAPGKGLEWVSAILDHGGSTYYAESVKGRFTISRDN |
| | | AKNTLYLQMSSLRAEDTAVYYCARVVYHAGGGVTY |
| | | DYRGQGTQVTVSS |
| SEQ ID NO: | BGA-5164 | GAGGTCCAGTTACTTGAGAGTGGTGGAGGTCTGGTC |
| 210 | DNA | CAACCAGGAGGTTCGCTGCGTTTATCCTGCGCCGCG |
| | | TCTGGATTCACGCTTTCCGCCGAAGACGTGGGTTGG |
| | | GTGCGTCAAGCGCCGGGGAAAGGACTGGAATGGGT |
| | | CTCCGCCATCTTGGATCATGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTGTCTACCATGCTGGTGGTGGCGTCAC |
| | | CTATGATTATCGGGGACAGGGCACCCAAGTTACAGT |
| | | CTCATCG |
| SEQ ID NO: | BGA-3953 | EVQLLESGGGLVQPGGSLRLSCAASGFTVSAEDVGWV |
| 211 | AA | RQAPGKGLEWVSAILDSGGSTYYAESVKGRFTISRDN |
| | | AKNTLYLQMSSLRAEDTAVYYCARIVYHAGGGVTFD |
| | | TRGQGTQVTVSS |
| SEQ ID NO: | BGA-3953 | GAGGTCCAGTTACTTGAGAGTGGTGGAGGTCTGGTC |
| 212 | DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATtcGGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCATTGTCTACCATGCTGGTGGTGGCGTCACC |
| | | TTTGATACTCGGGGACAGGGCACCCAAGTTACAGTC |
| | | TCATCG |
| SEQ ID NO: | BGA-5385 | EVQLLESGGGLVQPGGSLRLSCAASGFTLSAEDVGWV |
| 213 | AA | RQAPGKGLEWVSAILDHGGSTYYAESVKGRFTISRDN |
| | | AKNTLYLQMSSLRAEDTAVYYCARVIYHAGGGVTFD |
| | | TRGQGTQVTVSS |
| SEQ ID NO: | BGA-5385 | GAGGTCCAGTTACTTGAGAGTGGTGGAGGTCTGGTC |
| 214 | DNA | CAACCAGGAGGTTCGCTGCGTTTATCCTGCGCCGCG |
| | | TCTGGATTCACGCTTTCCGCCGAAGACGTGGGTTGG |
| | | GTGCGTCAAGCGCCGGGGAAAGGACTGGAATGGGT |
| | | CTCCGCCATCTTGGATCATGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTATCTACCATGCTGGTGGTGGCGTCAC |
| | | CTTTGATACTCGGGGACAGGGCACCCAAGTTACAGT |
| | | CTCATCG |
| SEQ ID NO: | BGA-9468 | EVQLLESGGGLVQPGGSLRLSCAASGFTVSAEDVGWV |
| 215 | AA | RQAPGKGLEWVSAILDSGGSTYYAESVKGRFTISRDN |
| | | AKNTLYLQMSSLRAEDTAVYYCARVVYHSGGGVTFD |
| | | LRGQGTQVTVSS |
| SEQ ID NO: | BGA-9468 | GAGGTCCAGTTACTTGAGAGTGGTGGAGGTCTGGTC |
| 216 | DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATtcGGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCGTTGTCTACCATTCTGGTGGTGGCGTCACC |
| | | TTTGATCTTCGGGGACAGGGCACCCAAGTTACAGTC |
| | | TCATCG |
| SEQ ID NO: | BGA-3285 | EVQLLESGGGLVQPGGSLRLSCAASGFTISAEDVGWV |
| 217 | AA | RQAPGKGLEWVSAILDFGGSTYYAESVKGRFTISRDN |
| | | AKNTLYLQMSSLRAEDTAVYYCARVVYHAGGGVTFD |
| | | TRGQGTQVTVSS |
| SEQ ID NO: | BGA-3285 | GAGGTCCAGTTACTTGAGAGTGGTGGAGGTCTGGTC |
| 218 | DNA | CAACCAGGAGGTTCGCTGCGTTTATCCTGCGCCGCG |
| | | TCTGGATTCACGATTTCCGCCGAAGACGTGGGTTGG |
| | | GTGCGTCAAGCGCCGGGGAAAGGACTGGAATGGGT |
| | | CTCCGCCATCTTGGATTTTGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCGTTGTCTACCATGCTGGTGGTGGCGTCACC |
| | | TTTGATACTCGGGGACAGGGCACCCAAGTTACAGTC |
| | | TCATCG |
| SEQ ID NO: | BGA-9442 | EVQLLESGGGLVQPGGSLRLSCAASGFTISAEDVGWV |
| 219 | AA | RQAPGKGLEWVSAILDSGGSTYYAESVKGRFTISRDN |
| | | AKNTLYLQMSSLRAEDTAVYYCARYVYHAGGGVTFD |
| | | LRGQGTQVTVSS |
| SEQ ID NO: | BGA-9442 | GAGGTCCAGTTACTTGAGAGTGGTGGAGGTCTGGTC |
| 220 | DNA | CAACCAGGAGGTTCGCTGCGTTTATCCTGCGCCGCG |
| | | TCTGGATTCACGATTTCCGCCGAAGACGTGGGTTGG |
| | | GTGCGTCAAGCGCCGGGGAAAGGACTGGAATGGGT |
| | | CTCCGCCATCTTGGATTCTGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCTATGTCTACCATGCTGGTGGTGGCGTCACC |
| | | TTTGATCTTCGGGGACAGGGCACCCAAGTTACAGTC |
| | | TCATCG |
| SEQ ID NO: | BGA-7746 | EVQLLESGGGLVQPGGSLRLSCAASGFTVSAEDVGWV |
| 221 | AA | RQAPGKGLEWVSAILDFGGSTYYAESVKGRFTISRDN |
| | | AKNTLYLQMSSLRAEDTAVYYCARVVYHAGGGVTFD |
| | | TRGQGTQVTVSS |
| SEQ ID NO: | BGA-7746 | GAGGTCCAGTTACTTGAGAGTGGTGGAGGTCTGGTC |
| 222 | DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATTTTGGTGGTTCGACATACTAT |
| | | GCAGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCGTCGTCTACCATGCTGGTGGTGGCGTCACC |
| | | TTTGATACGCGGGGACAGGGCACCCAAGTTACAGTC |
| | | TCATCG |
| SEQ ID NO: | BGA-8425 | EVQLLESGGGLVQPGGSLRLSCAASGFTLSAEDVGWV |
| 223 | AA | RQAPGKGLEWVSAILDSGGSTYYAESVKGRFTISRDN |
| | | AKNTLYLQMSSLRAEDTAVYYCARVVYHAGGGVTY |
| | | DYRGQGTQVTVSS |
| SEQ ID NO: | BGA-8425 | GAGGTCCAGTTACTTGAGAGTGGTGGAGGTCTGGTC |
| 224 | DNA | CAACCAGGAGGTTCGCTGCGTTTATCCTGCGCCGCG |
| | | TCTGGATTCACGCTGTCCGCCGAAGACGTGGGTTGG |
| | | GTGCGTCAAGCGCCGGGGAAAGGACTGGAATGGGT |
| | | CTCCGCCATCTTGGATTCGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTCGTCTACCATGCTGGTGGTGGCGTCAC |
| | | CTATGATTACCGGGGACAGGGCACCCAAGTTACAGT |
| | | CTCATCG |
| SEQ ID NO: | BGA-3386 | EVQLLESGGGLVQPGGSLRLSCAASGFTVSAEDVGWV |
| 225 | AA | RQAPGKGLEWVSAILDKGGSTYYAESVKGRFTISRDN |
| | | AKNTLYLQMSSLRAEDTAVYYCARIVYHAGGGVTFD |
| | | TWGQGTLVTVSS |
| SEQ ID NO: | BGA-3386 | GAGGTCCAGTTACTTGAGAGTGGTGGAGGTCTGGTC |
| 226 | DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATAAGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCATTGTCTACCATGCTGGTGGTGGCGTCAC |
| | | CTTTGATACTTGGGGACAGGGCACCTTAGTTACAGT |
| | | CTCATCG |
| SEQ ID NO: | BGA-5623- | EVQLLESGGGLVQPGGSLRLSCAASGFTVSAEDVGWV |
| 227 | mutFc-AA | RQAPGKGLEWVSAILDKGGSTYYAESVKGRFTISRDN |
| | | AKNTLYLQMSSLRAEDTAVYYCARIVYHAGGGVTFD |
| | | TRGQGTQVTVSSGGGGSDKTHTCPPCPAPPAAGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALAAPIEKTISKAKGQPREPQVYTLPP |
| | | SREEMTKNQVSLTCLVKGFYPSDIAVEWESNGQPENN |
| | | YKTTPPVLDSDGSFFLYSKLTVDKSRWQQGNVFSCSV |
| | | MHEALHNHYTQKSLSLSPGK |
| SEQ ID NO: | BGA-5623- | GAGGTCCAGTTACTTGAGAGTGGTGGAGGTCTGGTC |
| 228 | mutFc DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATAAGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCATTGTCTACCATGCTGGTGGTGGCGTCAC |
| | | CTTTGATACTCGGGGACAGGGCACCCAAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |
| SEQ ID NO: | BGA-2690- | EVQLLESGGGLVQPGGSLRLSCAASGFTLSREDVGWV |
| 229 | mutFc AA | RQAPGKGLEWVSAILDFGSSTYYAESVKGRFTISRDNA |
| | | KNTLYLQMSSLRAEDTAVYYCARVVYHAGGGVTYD |
| | | YRGQGTQVTVSSGGGGSDKTHTCPPCPAPPAAGPSVF |
| | | LFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALAAPIEKTISKAKGQPREPQVYTLP |
| | | PSREEMTKNQVSLTCLVKGFYPSDIAVEWESNGQPEN |
| | | NYKTTPPVLDSDGSFFLYSKLTVDKSRWQQGNVFSCS |
| | | VMHEALHNHYTQKSLSLSPGK |
| SEQ ID NO: | BGA-2690 | GAGGTCCAGTTACTTGAGAGTGGTGGAGGTCTGGTC |
| 230 | mutFc DNA | CAACCAGGAGGTTCGCTGCGTTTATCCTGCGCCGCG |
| | | TCTGGATTCACGCTTTCCAGAGAAGACGTGGGTTGG |
| | | GTGCGTCAAGCGCCGGGGAAAGGACTGGAATGGGT |
| | | CTCCGCCATCTTGGATTTCGGTTCTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCGTTGTCTACCATGCTGGTGGTGGCGTCACC |
| | | TATGATTATCGGGGACAGGGCACCCAAGTTACAGTC |
| | | TCATCGGGCGGCGGAGGGTCTGACAAGACCCACAC |
| | | ATGCCCCCCTTGTCCTGCTCCACCAGCTGCAGGACC |
| | | AAGCGTGTTCCTGTTTCCACCCAAGCCCAAGGATAC |
| | | CCTGATGATCTCTCGGACCCCAGAGGTGACATGCGT |
| | | GGTGGTGGATGTGAGCCACGAGGACCCCGAGGTGA |
| | | AGTTCAACTGGTATGTGGACGGCGTGGAGGTGCACA |
| | | ATGCTAAGACCAAGCCCAGGGAGGAGCAGTACAAC |
| | | TCCACCTATAGAGTGGTGTCTGTGCTGACAGTGCTG |
| | | CACCAGGATTGGCTGAACGGCAAGGAGTATAAGTG |
| | | CAAGGTGTCCAATAAGGCCCTGGCCGCTCCTATCGA |
| | | GAAGACCATCTCTAAGGCCAAGGGCCAGCCCAGAG |
| | | AGCCTCAGGTGTACACACTGCCTCCATCCCGGGAAG |
| | | AGATGACCAAGAACCAGGTGTCTCTGACATGTCTGG |
| | | TCAAGGGCTTCTATCCCTCTGACATCGCCGTGGAGT |
| | | GGGAGAGCAATGGCCAGCCTGAGAACAATTACAAG |
| | | ACCACACCCCCTGTGCTGGATTCCGACGGCTCTTTCT |
| | | TTCTGTATAGCAAGCTGACCGTGGACAAGTCCCGGT |
| | | GGCAGCAGGGCAACGTGTTCAGCTGTTCCGTGATGC |
| | | ACGAAGCTCTGCATAATCACTATACTCAGAAATCCC |
| | | TGTCACTGTCACCTGGTAAA |
| SEQ ID NO: | BGA-3849- | EVQLLESGGGLVQPGGSLRLSCAASGFTLSHEDVGWV |
| 231 | mutFc | RQAPGKGLEWVSAILDSGGSTYYAESVKGRFTISRDN |
| | AA | AKNTLYLQMSSLRAEDTAVYYCARYVYHAGGGVTFD |
| | | TRGQGTQVTVSSGGGGSDKTHTCPPCPAPPAAGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALAAPIEKTISKAKGQPREPQVYTLPP |
| | | SREEMTKNQVSLTCLVKGFYPSDIAVEWESNGQPENN |
| | | YKTTPPVLDSDGSFFLYSKLTVDKSRWQQGNVFSCSV |
| | | MHEALHNHYTQKSLSLSPGK |
| SEQ ID NO: | BGA-3849 | GAGGTCCAGTTACTTGAGAGTGGTGGAGGTCTGGTC |
| 232 | mutFc DNA | CAACCAGGAGGTTCGCTGCGTTTATCCTGCGCCGCG |
| | | TCTGGATTCACGCTTTCCCATGAAGACGTGGGTTGG |
| | | GTGCGTCAAGCGCCGGGGAAAGGACTGGAATGGGT |
| | | CTCCGCCATCTTGGATTCCGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCTATGTCTACCATGCTGGTGGTGGCGTCACC |
| | | TTTGATACTCGGGGACAGGGCACCCAAGTTACAGTC |
| | | TCATCGGGCGGCGGAGGGTCTGACAAGACCCACAC |
| | | ATGCCCCCCTTGTCCTGCTCCACCAGCTGCAGGACC |
| | | AAGCGTGTTCCTGTTTCCACCCAAGCCCAAGGATAC |
| | | CCTGATGATCTCTCGGACCCCAGAGGTGACATGCGT |
| | | GGTGGTGGATGTGAGCCACGAGGACCCCGAGGTGA |
| | | AGTTCAACTGGTATGTGGACGGCGTGGAGGTGCACA |
| | | ATGCTAAGACCAAGCCCAGGGAGGAGCAGTACAAC |
| | | TCCACCTATAGAGTGGTGTCTGTGCTGACAGTGCTG |
| | | CACCAGGATTGGCTGAACGGCAAGGAGTATAAGTG |
| | | CAAGGTGTCCAATAAGGCCCTGGCCGCTCCTATCGA |
| | | GAAGACCATCTCTAAGGCCAAGGGCCAGCCCAGAG |
| | | AGCCTCAGGTGTACACACTGCCTCCATCCCGGGAAG |
| | | AGATGACCAAGAACCAGGTGTCTCTGACATGTCTGG |
| | | TCAAGGGCTTCTATCCCTCTGACATCGCCGTGGAGT |
| | | GGGAGAGCAATGGCCAGCCTGAGAACAATTACAAG |
| | | ACCACACCCCCTGTGCTGGATTCCGACGGCTCTTTCT |
| | | TTCTGTATAGCAAGCTGACCGTGGACAAGTCCCGGT |
| | | GGCAGCAGGGCAACGTGTTCAGCTGTTCCGTGATGC |
| | | ACGAAGCTCTGCATAATCACTATACTCAGAAATCCC |
| | | TGTCACTGTCACCTGGTAAA |
| SEQ ID NO: | BGA-7916- | EVQLLESGGGLVQPGGSLRLSCAASGFTVSAEDVGWV |
| 233 | mutFc AA | RQAPGKGLEWVSAILDSGGSTYYAESVKGRFTISRDN |
| | | AKNTLYLQMSSLRAEDTAVYYCARVVYHAGGGVTY |
| | | DTRGQGTQVTVSSGGGGSDKTHTCPPCPAPPAAGPSV |
| | | FLFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALAAPIEKTISKAKGQPREPQVYTLP |
| | | PSREEMTKNQVSLTCLVKGFYPSDIAVEWESNGQPEN |
| | | NYKTTPPVLDSDGSFFLYSKLTVDKSRWQQGNVFSCS |
| | | VMHEALHNHYTQKSLSLSPGK |
| SEQ ID NO: | BGA-7916- | GAGGTCCAGTTACTTGAGAGTGGTGGAGGTCTGGTC |
| 234 | mutFc DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATTCGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTGTCTACCATGCTGGTGGTGGCGTCAC |
| | | CTATGATACTCGGGGACAGGGCACCCAAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |
| SEQ ID NO: | BGA-4988- | EVQLLESGGGLVQPGGSLRLSCAASGFTVSAEDVGWV |
| 235 | mutFc AA | RQAPGKGLEWVSAILDRGGSTYYAESVKGRFTISRDN |
| | | AKNTLYLQMSSLRAEDTAVYYCARVVYHAGGGVTY |
| | | DTRGQGTQVTVSSGGGGSDKTHTCPPCPAPPAAGPSV |
| | | FLFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALAAPIEKTISKAKGQPREPQVYTLP |
| | | PSREEMTKNQVSLTCLVKGFYPSDIAVEWESNGQPEN |
| | | NYKTTPPVLDSDGSFFLYSKLTVDKSRWQQGNVFSCS |
| | | VMHEALHNHYTQKSLSLSPGK |
| SEQ ID NO: | BGA-4988- | GAGGTCCAGTTACTTGAGAGTGGTGGAGGTCTGGTC |
| 236 | mutFc DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATCGGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTGTCTACCATGCTGGTGGTGGCGTCAC |
| | | CTATGATACTCGGGGACAGGGCACCCAAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |
| SEQ ID NO: | BGA-5164- | EVQLLESGGGLVQPGGSLRLSCAASGFTLSAEDVGWV |
| 237 | mutFc AA | RQAPGKGLEWVSAILDHGGSTYYAESVKGRFTISRDN |
| | | AKNTLYLQMSSLRAEDTAVYYCARVVYHAGGGVTY |
| | | DYRGQGTQVTVSSGGGGSDKTHTCPPCPAPPAAGPSV |
| | | FLFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALAAPIEKTISKAKGQPREPQVYTLP |
| | | PSREEMTKNQVSLTCLVKGFYPSDIAVEWESNGQPEN |
| | | NYKTTPPVLDSDGSFFLYSKLTVDKSRWQQGNVFSCS |
| | | VMHEALHNHYTQKSLSLSPGK |
| SEQ ID NO: | BGA-5164- | GAGGTCCAGTTACTTGAGAGTGGTGGAGGTCTGGTC |
| 238 | mutFc | CAACCAGGAGGTTCGCTGCGTTTATCCTGCGCCGCG |
| | DNA | TCTGGATTCACGCTTTCCGCCGAAGACGTGGGTTGG |
| | | GTGCGTCAAGCGCCGGGGAAAGGACTGGAATGGGT |
| | | CTCCGCCATCTTGGATCATGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTGTCTACCATGCTGGTGGTGGCGTCAC |
| | | CTATGATTATCGGGGACAGGGCACCCAAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |
| SEQ ID NO: | BGA-3953- | EVQLLESGGGLVQPGGSLRLSCAASGFTVSAEDVGWV |
| 239 | mutFc AA | RQAPGKGLEWVSAILDSGGSTYYAESVKGRFTISRDN |
| | | AKNTLYLQMSSLRAEDTAVYYCARIVYHAGGGVTFD |
| | | TRGQGTQVTVSSGGGGSDKTHTCPPCPAPPAAGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALAAPIEKTISKAKGQPREPQVYTLPP |
| | | SREEMTKNQVSLTCLVKGFYPSDIAVEWESNGQPENN |
| | | YKTTPPVLDSDGSFFLYSKLTVDKSRWQQGNVFSCSV |
| | | MHEALHNHYTQKSLSLSPGK |
| SEQ ID NO: | BGA-3953- | GAGGTCCAGTTACTTGAGAGTGGTGGAGGTCTGGTC |
| 240 | mutFc DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATtcGGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCATTGTCTACCATGCTGGTGGTGGCGTCACC |
| | | TTTGATACTCGGGGACAGGGCACCCAAGTTACAGTC |
| | | TCATCGGGCGGCGGAGGGTCTGACAAGACCCACAC |
| | | ATGCCCCCCTTGTCCTGCTCCACCAGCTGCAGGACC |
| | | AAGCGTGTTCCTGTTTCCACCCAAGCCCAAGGATAC |
| | | CCTGATGATCTCTCGGACCCCAGAGGTGACATGCGT |
| | | GGTGGTGGATGTGAGCCACGAGGACCCCGAGGTGA |
| | | AGTTCAACTGGTATGTGGACGGCGTGGAGGTGCACA |
| | | ATGCTAAGACCAAGCCCAGGGAGGAGCAGTACAAC |
| | | TCCACCTATAGAGTGGTGTCTGTGCTGACAGTGCTG |
| | | CACCAGGATTGGCTGAACGGCAAGGAGTATAAGTG |
| | | CAAGGTGTCCAATAAGGCCCTGGCCGCTCCTATCGA |
| | | GAAGACCATCTCTAAGGCCAAGGGCCAGCCCAGAG |
| | | AGCCTCAGGTGTACACACTGCCTCCATCCCGGGAAG |
| | | AGATGACCAAGAACCAGGTGTCTCTGACATGTCTGG |
| | | TCAAGGGCTTCTATCCCTCTGACATCGCCGTGGAGT |
| | | GGGAGAGCAATGGCCAGCCTGAGAACAATTACAAG |
| | | ACCACACCCCCTGTGCTGGATTCCGACGGCTCTTTCT |
| | | TTCTGTATAGCAAGCTGACCGTGGACAAGTCCCGGT |
| | | GGCAGCAGGGCAACGTGTTCAGCTGTTCCGTGATGC |
| | | ACGAAGCTCTGCATAATCACTATACTCAGAAATCCC |
| | | TGTCACTGTCACCTGGTAAA |
| SEQ ID NO: | BGA-5385- | EVQLLESGGGLVQPGGSLRLSCAASGFTLSAEDVGWV |
| 241 | mutFc AA | RQAPGKGLEWVSAILDHGGSTYYAESVKGRFTISRDN |
| | | AKNTLYLQMSSLRAEDTAVYYCARVIYHAGGGVTYD |
| | | YRGQGTQVTVSSGGGGSDKTHTCPPCPAPPAAGPSVF |
| | | LFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALAAPIEKTISKAKGQPREPQVYTLP |
| | | PSREEMTKNQVSLTCLVKGFYPSDIAVEWESNGQPEN |
| | | NYKTTPPVLDSDGSFFLYSKLTVDKSRWQQGNVFSCS |
| | | VMHEALHNHYTQKSLSLSPGK |
| SEQ ID NO: | BGA-5385- | GAGGTCCAGTTACTTGAGAGTGGTGGAGGTCTGGTC |
| 242 | mutFc DNA | CAACCAGGAGGTTCGCTGCGTTTATCCTGCGCCGCG |
| | | TCTGGATTCACGcTTTCCGCCGAAGACGTGGGTTGG |
| | | GTGCGTCAAGCGCCGGGGAAAGGACTGGAATGGGT |
| | | CTCCGCCATCTTGGATCATGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTATCTACCATGCTGGTGGTGGCGTCAC |
| | | CTATGATTATCGGGGACAGGGCACCCAAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |
| SEQ ID NO: | BGA-9468- | EVQLLESGGGLVQPGGSLRLSCAASGFTVSAEDVGWV |
| 243 | mutFc AA | RQAPGKGLEWVSAILDSGGSTYYAESVKGRFTISRDN |
| | | AKNTLYLQMSSLRAEDTAVYYCARVVYHSGGGVTFD |
| | | LRGQGTQVTVSSGGGGSDKTHTCPPCPAPPAAGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALAAPIEKTISKAKGQPREPQVYTLPP |
| | | SREEMTKNQVSLTCLVKGFYPSDIAVEWESNGQPENN |
| | | YKTTPPVLDSDGSFFLYSKLTVDKSRWQQGNVFSCSV |
| | | MHEALHNHYTQKSLSLSPGK |
| SEQ ID NO: | BGA-9468- | GAGGTCCAGTTACTTGAGAGTGGTGGAGGTCTGGTC |
| 244 | mutFc DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATTCGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTGTCTACCATTCTGGTGGTGGCGTCAC |
| | | CTTTGATCTTCGGGGACAGGGCACCCAAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |
| SEQ ID NO: | BGA-3285- | EVQLLESGGGLVQPGGSLRLSCAASGFTISAEDVGWV |
| 245 | mutFc | RQAPGKGLEWVSAILDFGGSTYYAESVKGRFTISRDN |
| | AA | AKNTLYLQMSSLRAEDTAVYYCARVVYHAGGGVTFD |
| | | TRGQGTQVTVSSGGGGSDKTHTCPPCPAPPAAGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALAAPIEKTISKAKGQPREPQVYTLPP |
| | | SREEMTKNQVSLTCLVKGFYPSDIAVEWESNGQPENN |
| | | YKTTPPVLDSDGSFFLYSKLTVDKSRWQQGNVFSCSV |
| | | MHEALHNHYTQKSLSLSPGK |
| SEQ ID NO: | BGA-3285- | GAGGTCCAGTTACTTGAGAGTGGTGGAGGTCTGGTC |
| 246 | mutFc DNA | CAACCAGGAGGTTCGCTGCGTTTATCCTGCGCCGCG |
| | | TCTGGATTCACGATTTCCGCCGAAGACGTGGGTTGG |
| | | GTGCGTCAAGCGCCGGGGAAAGGACTGGAATGGGT |
| | | CTCCGCCATCTTGGATTTTGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCGTTGTCTACCATGCTGGTGGTGGCGTCACC |
| | | TTTGATACTCGGGGACAGGGCACCCAAGTTACAGTC |
| | | TCATCGGGCGGCGGAGGGTCTGACAAGACCCACAC |
| | | ATGCCCCCCTTGTCCTGCTCCACCAGCTGCAGGACC |
| | | AAGCGTGTTCCTGTTTCCACCCAAGCCCAAGGATAC |
| | | CCTGATGATCTCTCGGACCCCAGAGGTGACATGCGT |
| | | GGTGGTGGATGTGAGCCACGAGGACCCCGAGGTGA |
| | | AGTTCAACTGGTATGTGGACGGCGTGGAGGTGCACA |
| | | ATGCTAAGACCAAGCCCAGGGAGGAGCAGTACAAC |
| | | TCCACCTATAGAGTGGTGTCTGTGCTGACAGTGCTG |
| | | CACCAGGATTGGCTGAACGGCAAGGAGTATAAGTG |
| | | CAAGGTGTCCAATAAGGCCCTGGCCGCTCCTATCGA |
| | | GAAGACCATCTCTAAGGCCAAGGGCCAGCCCAGAG |
| | | AGCCTCAGGTGTACACACTGCCTCCATCCCGGGAAG |
| | | AGATGACCAAGAACCAGGTGTCTCTGACATGTCTGG |
| | | TCAAGGGCTTCTATCCCTCTGACATCGCCGTGGAGT |
| | | GGGAGAGCAATGGCCAGCCTGAGAACAATTACAAG |
| | | ACCACACCCCCTGTGCTGGATTCCGACGGCTCTTTCT |
| | | TTCTGTATAGCAAGCTGACCGTGGACAAGTCCCGGT |
| | | GGCAGCAGGGCAACGTGTTCAGCTGTTCCGTGATGC |
| | | ACGAAGCTCTGCATAATCACTATACTCAGAAATCCC |
| | | TGTCACTGTCACCTGGTAAA |
| SEQ ID NO: | BGA-9442- | EVQLLESGGGLVQPGGSLRLSCAASGFTISAEDVGWV |
| 247 | mutFc AA | RQAPGKGLEWVSAILDSGGSTYYAESVKGRFTISRDN |
| | | AKNTLYLQMSSLRAEDTAVYYCARYVYHAGGGVTFD |
| | | LRGQGTQVTVSSGGGGSDKTHTCPPCPAPPAAGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALAAPIEKTISKAKGQPREPQVYTLPP |
| | | SREEMTKNQVSLTCLVKGFYPSDIAVEWESNGQPENN |
| | | YKTTPPVLDSDGSFFLYSKLTVDKSRWQQGNVFSCSV |
| | | MHEALHNHYTQKSLSLSPGK |
| SEQ ID NO: | BGA-9442- | GAGGTCCAGTTACTTGAGAGTGGTGGAGGTCTGGTC |
| 248 | mutFc DNA | CAACCAGGAGGTTCGCTGCGTTTATCCTGCGCCGCG |
| | | TCTGGATTCACGATTTCCGCCGAAGACGTGGGTTGG |
| | | GTGCGTCAAGCGCCGGGGAAAGGACTGGAATGGGT |
| | | CTCCGCCATCTTGGATTCTGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCTATGTCTACCATGCTGGTGGTGGCGTCACC |
| | | TTTGATCTTCGGGGACAGGGCACCCAAGTTACAGTC |
| | | TCATCGGGCGGCGGAGGGTCTGACAAGACCCACAC |
| | | ATGCCCCCCTTGTCCTGCTCCACCAGCTGCAGGACC |
| | | AAGCGTGTTCCTGTTTCCACCCAAGCCCAAGGATAC |
| | | CCTGATGATCTCTCGGACCCCAGAGGTGACATGCGT |
| | | GGTGGTGGATGTGAGCCACGAGGACCCCGAGGTGA |
| | | AGTTCAACTGGTATGTGGACGGCGTGGAGGTGCACA |
| | | ATGCTAAGACCAAGCCCAGGGAGGAGCAGTACAAC |
| | | TCCACCTATAGAGTGGTGTCTGTGCTGACAGTGCTG |
| | | CACCAGGATTGGCTGAACGGCAAGGAGTATAAGTG |
| | | CAAGGTGTCCAATAAGGCCCTGGCCGCTCCTATCGA |
| | | GAAGACCATCTCTAAGGCCAAGGGCCAGCCCAGAG |
| | | AGCCTCAGGTGTACACACTGCCTCCATCCCGGGAAG |
| | | AGATGACCAAGAACCAGGTGTCTCTGACATGTCTGG |
| | | TCAAGGGCTTCTATCCCTCTGACATCGCCGTGGAGT |
| | | GGGAGAGCAATGGCCAGCCTGAGAACAATTACAAG |
| | | ACCACACCCCCTGTGCTGGATTCCGACGGCTCTTTCT |
| | | TTCTGTATAGCAAGCTGACCGTGGACAAGTCCCGGT |
| | | GGCAGCAGGGCAACGTGTTCAGCTGTTCCGTGATGC |
| | | ACGAAGCTCTGCATAATCACTATACTCAGAAATCCC |
| | | TGTCACTGTCACCTGGTAAA |
| SEQ ID NO: | BGA-7746- | EVQLLESGGGLVQPGGSLRLSCAASGFTVSAEDVGWV |
| 249 | mutFc AA | RQAPGKGLEWVSAILDFGGSTYYAESVKGRFTISRDN |
| | | AKNTLYLQMSSLRAEDTAVYYCARVVYHAGGGVTFD |
| | | TRGQGTQVTVSSGGGGSDKTHTCPPCPAPPAAGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALAAPIEKTISKAKGQPREPQVYTLPP |
| | | SREEMTKNQVSLTCLVKGFYPSDIAVEWESNGQPENN |
| | | YKTTPPVLDSDGSFFLYSKLTVDKSRWQQGNVFSCSV |
| | | MHEALHNHYTQKSLSLSPGK |
| SEQ ID NO: | BGA-7746- | GAGGTCCAGTTACTTGAGAGTGGTGGAGGTCTGGTC |
| 250 | mutFc DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATTTTGGTGGTTCGACATACTAT |
| | | GCAGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCGTCGTCTACCATGCTGGTGGTGGCGTCACC |
| | | TTTGATACGCGGGGACAGGGCACCCAAGTTACAGTC |
| | | TCATCGGGCGGCGGAGGGTCTGACAAGACCCACAC |
| | | ATGCCCCCCTTGTCCTGCTCCACCAGCTGCAGGACC |
| | | AAGCGTGTTCCTGTTTCCACCCAAGCCCAAGGATAC |
| | | CCTGATGATCTCTCGGACCCCAGAGGTGACATGCGT |
| | | GGTGGTGGATGTGAGCCACGAGGACCCCGAGGTGA |
| | | AGTTCAACTGGTATGTGGACGGCGTGGAGGTGCACA |
| | | ATGCTAAGACCAAGCCCAGGGAGGAGCAGTACAAC |
| | | TCCACCTATAGAGTGGTGTCTGTGCTGACAGTGCTG |
| | | CACCAGGATTGGCTGAACGGCAAGGAGTATAAGTG |
| | | CAAGGTGTCCAATAAGGCCCTGGCCGCTCCTATCGA |
| | | GAAGACCATCTCTAAGGCCAAGGGCCAGCCCAGAG |
| | | AGCCTCAGGTGTACACACTGCCTCCATCCCGGGAAG |
| | | AGATGACCAAGAACCAGGTGTCTCTGACATGTCTGG |
| | | TCAAGGGCTTCTATCCCTCTGACATCGCCGTGGAGT |
| | | GGGAGAGCAATGGCCAGCCTGAGAACAATTACAAG |
| | | ACCACACCCCCTGTGCTGGATTCCGACGGCTCTTTCT |
| | | TTCTGTATAGCAAGCTGACCGTGGACAAGTCCCGGT |
| | | GGCAGCAGGGCAACGTGTTCAGCTGTTCCGTGATGC |
| | | ACGAAGCTCTGCATAATCACTATACTCAGAAATCCC |
| | | TGTCACTGTCACCTGGTAAA |
| SEQ ID NO: | BGA-8425- | EVQLLESGGGLVQPGGSLRLSCAASGFTLSAEDVGWV |
| 2 51 | mutFc AA | RQAPGKGLEWVSAILDSGGSTYYAESVKGRFTISRDN |
| | | AKNTLYLQMSSLRAEDTAVYYCARVVYHAGGGVTY |
| | | DYRGQGTQVTVSSGGGGSDKTHTCPPCPAPPAAGPSV |
| | | FLFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALAAPIEKTISKAKGQPREPQVYTLP |
| | | PSREEMTKNQVSLTCLVKGFYPSDIAVEWESNGQPEN |
| | | NYKTTPPVLDSDGSFFLYSKLTVDKSRWQQGNVFSCS |
| | | VMHEALHNHYTQKSLSLSPGK |
| SEQ ID NO: | BGA-8425- | GAGGTCCAGTTACTTGAGAGTGGTGGAGGTCTGGTC |
| 252 | mutFc DNA | CAACCAGGAGGTTCGCTGCGTTTATCCTGCGCCGCG |
| | | TCTGGATTCACGCTGTCCGCCGAAGACGTGGGTTGG |
| | | GTGCGTCAAGCGCCGGGGAAAGGACTGGAATGGGT |
| | | CTCCGCCATCTTGGATTCGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTCGTCTACCATGCTGGTGGTGGCGTCAC |
| | | CTATGATTACCGGGGACAGGGCACCCAAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |
| SEQ ID NO: | BGA-3386- | EVQLLESGGGLVQPGGSLRLSCAASGFTVSAEDVGWV |
| 253 | mutFc AA | RQAPGKGLEWVSAILDKGGSTYYAESVKGRFTISRDN |
| | | AKNTLYLQMSSLRAEDTAVYYCARIVYHAGGGVTFD |
| | | TWGQGTLVTVSSGGGGSDKTHTCPPCPAPPAAGPSVF |
| | | LFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALAAPIEKTISKAKGQPREPQVYTLP |
| | | PSREEMTKNQVSLTCLVKGFYPSDIAVEWESNGQPEN |
| | | NYKTTPPVLDSDGSFFLYSKLTVDKSRWQQGNVFSCS |
| | | VMHEALHNHYTQKSLSLSPGK |
| SEQ ID NO: | BGA-3386- | GAGGTCCAGTTACTTGAGAGTGGTGGAGGTCTGGTC |
| 254 | mutFc | CAACCAGGAGGTTCGCTGCGTTTATCCTGCGCCGCG |
| | DNA | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATAAGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCATTGTCTACCATGCTGGTGGTGGCGTCAC |
| | | CTTTGATACTTGGGGACAGGGCACCTTAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |
| SEQ ID NO: | Format A | QVQLVQSGAEVKKPGASVKVSCKASGYIFTSYYLHW |
| 255 | BGA- | VRQAPGQGLEWIGYINPQTGKTSYAQKFQGRVTMTR |
| | 5623AA | DTSTSTVYMELSSLRSEDTAVYYCAREYGNYNYPLDY |
| | (BE-189) | WGQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPPAAGPSVFLFPPKPKDTLMISRTPEV |
| | | TCVVVDVSHEDPEVKFNWYVDGVEVHNAKTKPREEQ |
| | | YNSTYRVVSVLTVLHQDWLNGKEYKCKVSNKALAAP |
| | | IEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSL |
| | | SPGKGGGGSEVQLLESGGGLVQPGGSLRLSCAASGFT |
| | | VSAEDVGWVRQAPGKGLEWVSAILDKGGSTYYAESV |
| | | KGRFTISRDNAKNTLYLQMSSLRAEDTAVYYCARIVY |
| | | HAGGGVTFDTRGQGTQVTVSS |
| SEQ ID NO: | Format A | CAGGTGCAGCTGGTGCAGAGCGGCGCGGAAGTGAA |
| 256 | BGA-5623 | AAAACCGGGCGCGAGCGTGAAAGTGAGCTGCAAAG |
| | DNA | CGAGCGGCTATATTTTTACCAGCTATTACCTGCATTG |
| | | GGTGCGCCAGGCGCCGGGCCAGGGCCTGGAATGGA |
| | | TTGGCTATATTAACCCGCAGACCGGCAAGACCAGCT |
| | | ATGCCCAGAAATTTCAGGGCCGCGTGACCATGACCC |
| | | GCGATACCAGCACCAGCACCGTGTATATGGAACTGA |
| | | GCAGCCTGCGCAGCGAAGATACCGCGGTGTATTATT |
| | | GCGCGCGCGAATATGGCAACTATAACTATCCGCTGG |
| | | ATTATTGGGGCCAGGGCACCCTGGTGACCGTGAGCA |
| | | GCGCTAGCACCAAGGGGCCCTCGGTCTTCCCCCTGG |
| | | CACCCTCCTCCAAGAGTACTTCTGGGGGCACAGCGG |
| | | CCCTGGGCTGCCTGGTCAAGGACTACTTCCCCGAAC |
| | | CGGTGACGGTGTCGTGGAACTCAGGCGCCCTGACCA |
| | | GCGGCGTGCACACCTTCCCGGCTGTCCTACAGTCCT |
| | | CAGGACTCTACTCCCTCAGCAGCGTGGTGACCGTGC |
| | | CCTCCAGCAGCTTGGGCACCCAGACCTACATCTGCA |
| | | ACGTGAATCACAAGCCCAGCAACACCAAGGTGGAC |
| | | AAGAAAGTTGAGCCAAAGTCCTGTGACAAGACCCA |
| | | CACATGCCCCCCTTGTCCTGCTCCACCAGCTGCAGG |
| | | ACCAAGCGTGTTCCTGTTTCCACCCAAGCCCAAGGA |
| | | TACCCTGATGATCTCTCGGACCCCAGAGGTGACATG |
| | | CGTGGTGGTGGATGTGAGCCACGAGGACCCCGAGG |
| | | TGAAGTTCAACTGGTATGTGGACGGCGTGGAGGTGC |
| | | ACAATGCTAAGACCAAGCCCAGGGAGGAGCAGTAC |
| | | AACTCCACCTATAGAGTGGTGTCTGTGCTGACAGTG |
| | | CTGCACCAGGATTGGCTGAACGGCAAGGAGTATAA |
| | | GTGCAAGGTGTCCAATAAGGCCCTGGCCGCTCCTAT |
| | | CGAGAAGACCATCTCTAAGGCCAAGGGCCAGCCCA |
| | | GAGAGCCTCAGGTGTACACACTGCCTCCATCCCGGG |
| | | AAGAGATGACCAAGAACCAGGTGTCTCTGACATGTC |
| | | TGGTCAAGGGCTTCTATCCCTCTGACATCGCCGTGG |
| | | AGTGGGAGAGCAATGGCCAGCCTGAGAACAATTAC |
| | | AAGACCACACCCCCTGTGCTGGATTCCGACGGCTCT |
| | | TTCTTTCTGTATAGCAAGCTGACCGTGGACAAGTCC |
| | | CGGTGGCAGCAGGGCAACGTGTTCAGCTGTTCCGTG |
| | | ATGCACGAAGCTCTGCATAATCACTATACTCAGAAA |
| | | TCCCTGTCACTGTCACCTGGTAAAGGTGGAGGCGGT |
| | | TCAGAGGTCCAGTTACTTGAGAGTGGTGGAGGTCTG |
| | | GTCCAACCAGGAGGTTCGCTGCGTTTATCCTGCGCC |
| | | GCGTCTGGATTCACGGTTTCCGCCGAAGACGTGGGT |
| | | TGGGTGCGTCAAGCGCCGGGGAAAGGACTGGAATG |
| | | GGTCTCCGCCATCTTGGATAAGGGTGGTTCGACATA |
| | | CTATGCGGAAAGTGTCAAAGGGCGCTTTACGATCTC |
| | | GCGCGATAACGCAAAAAATACTCTTTACCTTCAAAT |
| | | GTCTAGCCTTCGTGCTGAGGACACTGCGGTGTACTA |
| | | CTGCGCCCGCATTGTCTACCATGCTGGTGGTGGCGT |
| | | CACCTTTGATACTCGGGGACAGGGCACCCAAGTTAC |
| | | AGTCTCATCG |
| SEQ ID NO: | Format A | QVQLVQSGAEVKKPGASVKVSCKASGYIFTSYYLHW |
| 257 | BGA-2690 | VRQAPGQGLEWIGYINPQTGKTSYAQKFQGRVTMTR |
| | AA | DTSTSTVYMELSSLRSEDTAVYYCAREYGNYNYPLDY |
| | | WGQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPPAAGPSVFLFPPKPKDTLMISRTPEV |
| | | TCVVVDVSHEDPEVKFNWYVDGVEVHNAKTKPREEQ |
| | | YNSTYRVVSVLTVLHQDWLNGKEYKCKVSNKALAAP |
| | | IEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSL |
| | | SPGKGGGGSEVQLLESGGGLVQPGGSLRLSCAASGFT |
| | | LSREDVGWVRQAPGKGLEWVSAILDFGSSTYYAESVK |
| | | GRFTISRDNAKNTLYLQMSSLRAEDTAVYYCARVVYH |
| | | AGGGVTYDYRGQGTQVTVSS |
| SEQ ID NO: | Format A | CAGGTGCAGCTGGTGCAGAGCGGCGCGGAAGTGAA |
| 258 | BGA-2690 | AAAACCGGGCGCGAGCGTGAAAGTGAGCTGCAAAG |
| | DNA | CGAGCGGCTATATTTTTACCAGCTATTACCTGCATTG |
| | | GGTGCGCCAGGCGCCGGGCCAGGGCCTGGAATGGA |
| | | TTGGCTATATTAACCCGCAGACCGGCAAGACCAGCT |
| | | ATGCCCAGAAATTTCAGGGCCGCGTGACCATGACCC |
| | | GCGATACCAGCACCAGCACCGTGTATATGGAACTGA |
| | | GCAGCCTGCGCAGCGAAGATACCGCGGTGTATTATT |
| | | GCGCGCGCGAATATGGCAACTATAACTATCCGCTGG |
| | | ATTATTGGGGCCAGGGCACCCTGGTGACCGTGAGCA |
| | | GCGCTAGCACCAAGGGGCCCTCGGTCTTCCCCCTGG |
| | | CACCCTCCTCCAAGAGTACTTCTGGGGGCACAGCGG |
| | | CCCTGGGCTGCCTGGTCAAGGACTACTTCCCCGAAC |
| | | CGGTGACGGTGTCGTGGAACTCAGGCGCCCTGACCA |
| | | GCGGCGTGCACACCTTCCCGGCTGTCCTACAGTCCT |
| | | CAGGACTCTACTCCCTCAGCAGCGTGGTGACCGTGC |
| | | CCTCCAGCAGCTTGGGCACCCAGACCTACATCTGCA |
| | | ACGTGAATCACAAGCCCAGCAACACCAAGGTGGAC |
| | | AAGAAAGTTGAGCCAAAGTCCTGTGACAAGACCCA |
| | | CACATGCCCCCCTTGTCCTGCTCCACCAGCTGCAGG |
| | | ACCAAGCGTGTTCCTGTTTCCACCCAAGCCCAAGGA |
| | | TACCCTGATGATCTCTCGGACCCCAGAGGTGACATG |
| | | CGTGGTGGTGGATGTGAGCCACGAGGACCCCGAGG |
| | | TGAAGTTCAACTGGTATGTGGACGGCGTGGAGGTGC |
| | | ACAATGCTAAGACCAAGCCCAGGGAGGAGCAGTAC |
| | | AACTCCACCTATAGAGTGGTGTCTGTGCTGACAGTG |
| | | CTGCACCAGGATTGGCTGAACGGCAAGGAGTATAA |
| | | GTGCAAGGTGTCCAATAAGGCCCTGGCCGCTCCTAT |
| | | CGAGAAGACCATCTCTAAGGCCAAGGGCCAGCCCA |
| | | GAGAGCCTCAGGTGTACACACTGCCTCCATCCCGGG |
| | | AAGAGATGACCAAGAACCAGGTGTCTCTGACATGTC |
| | | TGGTCAAGGGCTTCTATCCCTCTGACATCGCCGTGG |
| | | AGTGGGAGAGCAATGGCCAGCCTGAGAACAATTAC |
| | | AAGACCACACCCCCTGTGCTGGATTCCGACGGCTCT |
| | | TTCTTTCTGTATAGCAAGCTGACCGTGGACAAGTCC |
| | | CGGTGGCAGCAGGGCAACGTGTTCAGCTGTTCCGTG |
| | | ATGCACGAAGCTCTGCATAATCACTATACTCAGAAA |
| | | TCCCTGTCACTGTCACCTGGTAAAGGTGGAGGCGGT |
| | | TCAGAGGTCCAGTTACTTGAGAGTGGTGGAGGTCTG |
| | | GTCCAACCAGGAGGTTCGCTGCGTTTATCCTGCGCC |
| | | GCGTCTGGATTCACGCTTTCCAGAGAAGACGTGGGT |
| | | TGGGTGCGTCAAGCGCCGGGGAAAGGACTGGAATG |
| | | GGTCTCCGCCATCTTGGATTTCGGTTCTTCGACATAC |
| | | TATGCGGAAAGTGTCAAAGGGCGCTTTACGATCTCG |
| | | CGCGATAACGCAAAAAATACTCTTTACCTTCAAATG |
| | | TCTAGCCTTCGTGCTGAGGACACTGCGGTGTACTAC |
| | | TGCGCCCGCGTTGTCTACCATGCTGGTGGTGGCGTC |
| | | ACCTATGATTATCGGGGACAGGGCACCCAAGTTACA |
| | | GTCTCATCG |
| SEQ ID NO: | Format A | QVQLVQSGAEVKKPGASVKVSCKASGYIFTSYYLHW |
| 259 | BGA-3849 | VRQAPGQGLEWIGYINPQTGKTSYAQKFQGRVTMTR |
| | AA | DTSTSTVYMELSSLRSEDTAVYYCAREYGNYNYPLDY |
| | | WGQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPPAAGPSVFLFPPKPKDTLMISRTPEV |
| | | TCVVVDVSHEDPEVKFNWYVDGVEVHNAKTKPREEQ |
| | | YNSTYRVVSVLTVLHQDWLNGKEYKCKVSNKALAAP |
| | | IEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSL |
| | | SPGKGGGGSEVQLLESGGGLVQPGGSLRLSCAASGFT |
| | | LSHEDVGWVRQAPGKGLEWVSAILDSGGSTYYAESV |
| | | KGRFTISRDNAKNTLYLQMSSLRAEDTAVYYCARYVY |
| | | HAGGGVTFDTRGQGTQVTVSS |
| SEQ ID NO: | Format A | CAGGTGCAGCTGGTGCAGAGCGGCGCGGAAGTGAA |
| 260 | BGA-3849 | AAAACCGGGCGCGAGCGTGAAAGTGAGCTGCAAAG |
| | DNA | CGAGCGGCTATATTTTTACCAGCTATTACCTGCATTG |
| | | GGTGCGCCAGGCGCCGGGCCAGGGCCTGGAATGGA |
| | | TTGGCTATATTAACCCGCAGACCGGCAAGACCAGCT |
| | | ATGCCCAGAAATTTCAGGGCCGCGTGACCATGACCC |
| | | GCGATACCAGCACCAGCACCGTGTATATGGAACTGA |
| | | GCAGCCTGCGCAGCGAAGATACCGCGGTGTATTATT |
| | | GCGCGCGCGAATATGGCAACTATAACTATCCGCTGG |
| | | ATTATTGGGGCCAGGGCACCCTGGTGACCGTGAGCA |
| | | GCGCTAGCACCAAGGGGCCCTCGGTCTTCCCCCTGG |
| | | CACCCTCCTCCAAGAGTACTTCTGGGGGCACAGCGG |
| | | CCCTGGGCTGCCTGGTCAAGGACTACTTCCCCGAAC |
| | | CGGTGACGGTGTCGTGGAACTCAGGCGCCCTGACCA |
| | | GCGGCGTGCACACCTTCCCGGCTGTCCTACAGTCCT |
| | | CAGGACTCTACTCCCTCAGCAGCGTGGTGACCGTGC |
| | | CCTCCAGCAGCTTGGGCACCCAGACCTACATCTGCA |
| | | ACGTGAATCACAAGCCCAGCAACACCAAGGTGGAC |
| | | AAGAAAGTTGAGCCAAAGTCCTGTGACAAGACCCA |
| | | CACATGCCCCCCTTGTCCTGCTCCACCAGCTGCAGG |
| | | ACCAAGCGTGTTCCTGTTTCCACCCAAGCCCAAGGA |
| | | TACCCTGATGATCTCTCGGACCCCAGAGGTGACATG |
| | | CGTGGTGGTGGATGTGAGCCACGAGGACCCCGAGG |
| | | TGAAGTTCAACTGGTATGTGGACGGCGTGGAGGTGC |
| | | ACAATGCTAAGACCAAGCCCAGGGAGGAGCAGTAC |
| | | AACTCCACCTATAGAGTGGTGTCTGTGCTGACAGTG |
| | | CTGCACCAGGATTGGCTGAACGGCAAGGAGTATAA |
| | | GTGCAAGGTGTCCAATAAGGCCCTGGCCGCTCCTAT |
| | | CGAGAAGACCATCTCTAAGGCCAAGGGCCAGCCCA |
| | | GAGAGCCTCAGGTGTACACACTGCCTCCATCCCGGG |
| | | AAGAGATGACCAAGAACCAGGTGTCTCTGACATGTC |
| | | TGGTCAAGGGCTTCTATCCCTCTGACATCGCCGTGG |
| | | AGTGGGAGAGCAATGGCCAGCCTGAGAACAATTAC |
| | | AAGACCACACCCCCTGTGCTGGATTCCGACGGCTCT |
| | | TTCTTTCTGTATAGCAAGCTGACCGTGGACAAGTCC |
| | | CGGTGGCAGCAGGGCAACGTGTTCAGCTGTTCCGTG |
| | | ATGCACGAAGCTCTGCATAATCACTATACTCAGAAA |
| | | TCCCTGTCACTGTCACCTGGTAAAGGTGGAGGCGGT |
| | | TCAGAGGTCCAGTTACTTGAGAGTGGTGGAGGTCTG |
| | | GTCCAACCAGGAGGTTCGCTGCGTTTATCCTGCGCC |
| | | GCGTCTGGATTCACGCTTTCCCATGAAGACGTGGGT |
| | | TGGGTGCGTCAAGCGCCGGGGAAAGGACTGGAATG |
| | | GGTCTCCGCCATCTTGGATTCCGGTGGTTCGACATA |
| | | CTATGCGGAAAGTGTCAAAGGGCGCTTTACGATCTC |
| | | GCGCGATAACGCAAAAAATACTCTTTACCTTCAAAT |
| | | GTCTAGCCTTCGTGCTGAGGACACTGCGGTGTACTA |
| | | CTGCGCCCGCTATGTCTACCATGCTGGTGGTGGCGT |
| | | CACCTTTGATACTCGGGGACAGGGCACCCAAGTTAC |
| | | AGTCTCATCG |
| SEQ ID NO: | Format A | QVQLVQSGAEVKKPGASVKVSCKASGYIFTSYYLHW |
| 261 | BGA-7916 | VRQAPGQGLEWIGYINPQTGKTSYAQKFQGRVTMTR |
| | AA | DTSTSTVYMELSSLRSEDTAVYYCAREYGNYNYPLDY |
| | | WGQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPPAAGPSVFLFPPKPKDTLMISRTPEV |
| | | TCVVVDVSHEDPEVKFNWYVDGVEVHNAKTKPREEQ |
| | | YNSTYRVVSVLTVLHQDWLNGKEYKCKVSNKALAAP |
| | | IEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSL |
| | | SPGKGGGGSEVQLLESGGGLVQPGGSLRLSCAASGFT |
| | | VSAEDVGWVRQAPGKGLEWVSAILDSGGSTYYAESV |
| | | KGRFTISRDNAKNTLYLQMSSLRAEDTAVYYCARVVY |
| | | HAGGGVTYDYRGQGTQVTVSS |
| SEQ ID NO: | Format A | CAGGTGCAGCTGGTGCAGAGCGGCGCGGAAGTGAA |
| 262 | BGA-7916 | AAAACCGGGCGCGAGCGTGAAAGTGAGCTGCAAAG |
| | DNA | CGAGCGGCTATATTTTTACCAGCTATTACCTGCATTG |
| | | GGTGCGCCAGGCGCCGGGCCAGGGCCTGGAATGGA |
| | | TTGGCTATATTAACCCGCAGACCGGCAAGACCAGCT |
| | | ATGCCCAGAAATTTCAGGGCCGCGTGACCATGACCC |
| | | GCGATACCAGCACCAGCACCGTGTATATGGAACTGA |
| | | GCAGCCTGCGCAGCGAAGATACCGCGGTGTATTATT |
| | | GCGCGCGCGAATATGGCAACTATAACTATCCGCTGG |
| | | ATTATTGGGGCCAGGGCACCCTGGTGACCGTGAGCA |
| | | GCGCTAGCACCAAGGGGCCCTCGGTCTTCCCCCTGG |
| | | CACCCTCCTCCAAGAGTACTTCTGGGGGCACAGCGG |
| | | CCCTGGGCTGCCTGGTCAAGGACTACTTCCCCGAAC |
| | | CGGTGACGGTGTCGTGGAACTCAGGCGCCCTGACCA |
| | | GCGGCGTGCACACCTTCCCGGCTGTCCTACAGTCCT |
| | | CAGGACTCTACTCCCTCAGCAGCGTGGTGACCGTGC |
| | | CCTCCAGCAGCTTGGGCACCCAGACCTACATCTGCA |
| | | ACGTGAATCACAAGCCCAGCAACACCAAGGTGGAC |
| | | AAGAAAGTTGAGCCAAAGTCCTGTGACAAGACCCA |
| | | CACATGCCCCCCTTGTCCTGCTCCACCAGCTGCAGG |
| | | ACCAAGCGTGTTCCTGTTTCCACCCAAGCCCAAGGA |
| | | TACCCTGATGATCTCTCGGACCCCAGAGGTGACATG |
| | | CGTGGTGGTGGATGTGAGCCACGAGGACCCCGAGG |
| | | TGAAGTTCAACTGGTATGTGGACGGCGTGGAGGTGC |
| | | ACAATGCTAAGACCAAGCCCAGGGAGGAGCAGTAC |
| | | AACTCCACCTATAGAGTGGTGTCTGTGCTGACAGTG |
| | | CTGCACCAGGATTGGCTGAACGGCAAGGAGTATAA |
| | | GTGCAAGGTGTCCAATAAGGCCCTGGCCGCTCCTAT |
| | | CGAGAAGACCATCTCTAAGGCCAAGGGCCAGCCCA |
| | | GAGAGCCTCAGGTGTACACACTGCCTCCATCCCGGG |
| | | AAGAGATGACCAAGAACCAGGTGTCTCTGACATGTC |
| | | TGGTCAAGGGCTTCTATCCCTCTGACATCGCCGTGG |
| | | AGTGGGAGAGCAATGGCCAGCCTGAGAACAATTAC |
| | | AAGACCACACCCCCTGTGCTGGATTCCGACGGCTCT |
| | | TTCTTTCTGTATAGCAAGCTGACCGTGGACAAGTCC |
| | | CGGTGGCAGCAGGGCAACGTGTTCAGCTGTTCCGTG |
| | | ATGCACGAAGCTCTGCATAATCACTATACTCAGAAA |
| | | TCCCTGTCACTGTCACCTGGTAAAGGTGGAGGCGGT |
| | | TCAGAGGTCCAGTTACTTGAGAGTGGTGGAGGTCTG |
| | | GTCCAACCAGGAGGTTCGCTGCGTTTATCCTGCGCC |
| | | GCGTCTGGATTCACGGTTTCCGCCGAAGACGTGGGT |
| | | TGGGTGCGTCAAGCGCCGGGGAAAGGACTGGAATG |
| | | GGTCTCCGCCATCTTGGATTCGGGTGGTTCGACATA |
| | | CTATGCGGAAAGTGTCAAAGGGCGCTTTACGATCTC |
| | | GCGCGATAACGCAAAAAATACTCTTTACCTTCAAAT |
| | | GTCTAGCCTTCGTGCTGAGGACACTGCGGTGTACTA |
| | | CTGCGCCCGCGTTGTCTACCATGCTGGTGGTGGCGT |
| | | CACCTATGATACTCGGGGACAGGGCACCCAAGTTAC |
| | | AGTCTCATCG |
| SEQ ID NO: | Format A | QVQLVQSGAEVKKPGASVKVSCKASGYIFTSYYLHW |
| 263 | BGA-4988 | VRQAPGQGLEWIGYINPQTGKTSYAQKFQGRVTMTR |
| | AA | DTSTSTVYMELSSLRSEDTAVYYCAREYGNYNYPLDY |
| | | WGQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPPAAGPSVFLFPPKPKDTLMISRTPEV |
| | | TCVVVDVSHEDPEVKFNWYVDGVEVHNAKTKPREEQ |
| | | YNSTYRVVSVLTVLHQDWLNGKEYKCKVSNKALAAP |
| | | IEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSL |
| | | SPGKGGGGSEVQLLESGGGLVQPGGSLRLSCAASGFT |
| | | VSAEDVGWVRQAPGKGLEWVSAILDRGGSTYYAESV |
| | | KGRFTISRDNAKNTLYLQMSSLRAEDTAVYYCARVVY |
| | | HAGGGVTYDYRGQGTQVTVSS |
| SEQ ID NO: | Format A | CAGGTGCAGCTGGTGCAGAGCGGCGCGGAAGTGAA |
| 264 | BGA-4988 | AAAACCGGGCGCGAGCGTGAAAGTGAGCTGCAAAG |
| | DNA | CGAGCGGCTATATTTTTACCAGCTATTACCTGCATTG |
| | | GGTGCGCCAGGCGCCGGGCCAGGGCCTGGAATGGA |
| | | TTGGCTATATTAACCCGCAGACCGGCAAGACCAGCT |
| | | ATGCCCAGAAATTTCAGGGCCGCGTGACCATGACCC |
| | | GCGATACCAGCACCAGCACCGTGTATATGGAACTGA |
| | | GCAGCCTGCGCAGCGAAGATACCGCGGTGTATTATT |
| | | GCGCGCGCGAATATGGCAACTATAACTATCCGCTGG |
| | | ATTATTGGGGCCAGGGCACCCTGGTGACCGTGAGCA |
| | | GCGCTAGCACCAAGGGGCCCTCGGTCTTCCCCCTGG |
| | | CACCCTCCTCCAAGAGTACTTCTGGGGGCACAGCGG |
| | | CCCTGGGCTGCCTGGTCAAGGACTACTTCCCCGAAC |
| | | CGGTGACGGTGTCGTGGAACTCAGGCGCCCTGACCA |
| | | GCGGCGTGCACACCTTCCCGGCTGTCCTACAGTCCT |
| | | CAGGACTCTACTCCCTCAGCAGCGTGGTGACCGTGC |
| | | CCTCCAGCAGCTTGGGCACCCAGACCTACATCTGCA |
| | | ACGTGAATCACAAGCCCAGCAACACCAAGGTGGAC |
| | | AAGAAAGTTGAGCCAAAGTCCTGTGACAAGACCCA |
| | | CACATGCCCCCCTTGTCCTGCTCCACCAGCTGCAGG |
| | | ACCAAGCGTGTTCCTGTTTCCACCCAAGCCCAAGGA |
| | | TACCCTGATGATCTCTCGGACCCCAGAGGTGACATG |
| | | CGTGGTGGTGGATGTGAGCCACGAGGACCCCGAGG |
| | | TGAAGTTCAACTGGTATGTGGACGGCGTGGAGGTGC |
| | | ACAATGCTAAGACCAAGCCCAGGGAGGAGCAGTAC |
| | | AACTCCACCTATAGAGTGGTGTCTGTGCTGACAGTG |
| | | CTGCACCAGGATTGGCTGAACGGCAAGGAGTATAA |
| | | GTGCAAGGTGTCCAATAAGGCCCTGGCCGCTCCTAT |
| | | CGAGAAGACCATCTCTAAGGCCAAGGGCCAGCCCA |
| | | GAGAGCCTCAGGTGTACACACTGCCTCCATCCCGGG |
| | | AAGAGATGACCAAGAACCAGGTGTCTCTGACATGTC |
| | | TGGTCAAGGGCTTCTATCCCTCTGACATCGCCGTGG |
| | | AGTGGGAGAGCAATGGCCAGCCTGAGAACAATTAC |
| | | AAGACCACACCCCCTGTGCTGGATTCCGACGGCTCT |
| | | TTCTTTCTGTATAGCAAGCTGACCGTGGACAAGTCC |
| | | CGGTGGCAGCAGGGCAACGTGTTCAGCTGTTCCGTG |
| | | ATGCACGAAGCTCTGCATAATCACTATACTCAGAAA |
| | | TCCCTGTCACTGTCACCTGGTAAAGGTGGAGGCGGT |
| | | TCAGAGGTCCAGTTACTTGAGAGTGGTGGAGGTCTG |
| | | GTCCAACCAGGAGGTTCGCTGCGTTTATCCTGCGCC |
| | | GCGTCTGGATTCACGGTTTCCGCCGAAGACGTGGGT |
| | | TGGGTGCGTCAAGCGCCGGGGAAAGGACTGGAATG |
| | | GGTCTCCGCCATCTTGGATCGGGGTGGTTCGACATA |
| | | CTATGCGGAAAGTGTCAAAGGGCGCTTTACGATCTC |
| | | GCGCGATAACGCAAAAAATACTCTTTACCTTCAAAT |
| | | GTCTAGCCTTCGTGCTGAGGACACTGCGGTGTACTA |
| | | CTGCGCCCGCGTTGTCTACCATGCTGGTGGTGGCGT |
| | | CACCTATGATACTCGGGGACAGGGCACCCAAGTTAC |
| | | AGTCTCATCG |
| SEQ ID NO: | Format A | QVQLVQSGAEVKKPGASVKVSCKASGYIFTSYYLHW |
| 265 | BGA-5164 | VRQAPGQGLEWIGYINPQTGKTSYAQKFQGRVTMTR |
| | AA | DTSTSTVYMELSSLRSEDTAVYYCAREYGNYNYPLDY |
| | | WGQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPPAAGPSVFLFPPKPKDTLMISRTPEV |
| | | TCVVVDVSHEDPEVKFNWYVDGVEVHNAKTKPREEQ |
| | | YNSTYRVVSVLTVLHQDWLNGKEYKCKVSNKALAAP |
| | | IEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSL |
| | | SPGKGGGGSEVQLLESGGGLVQPGGSLRLSCAASGFT |
| | | LSAEDVGWVRQAPGKGLEWVSAILDHGGSTYYAESV |
| | | KGRFTISRDNAKNTLYLQMSSLRAEDTAVYYCARVVY |
| | | HAGGGVTYDYRGQGTQVTVSS |
| SEQ ID NO: | Format A | CAGGTGCAGCTGGTGCAGAGCGGCGCGGAAGTGAA |
| 266 | BGA-5164 | AAAACCGGGCGCGAGCGTGAAAGTGAGCTGCAAAG |
| | DNA | CGAGCGGCTATATTTTTACCAGCTATTACCTGCATTG |
| | | GGTGCGCCAGGCGCCGGGCCAGGGCCTGGAATGGA |
| | | TTGGCTATATTAACCCGCAGACCGGCAAGACCAGCT |
| | | ATGCCCAGAAATTTCAGGGCCGCGTGACCATGACCC |
| | | GCGATACCAGCACCAGCACCGTGTATATGGAACTGA |
| | | GCAGCCTGCGCAGCGAAGATACCGCGGTGTATTATT |
| | | GCGCGCGCGAATATGGCAACTATAACTATCCGCTGG |
| | | ATTATTGGGGCCAGGGCACCCTGGTGACCGTGAGCA |
| | | GCGCTAGCACCAAGGGGCCCTCGGTCTTCCCCCTGG |
| | | CACCCTCCTCCAAGAGTACTTCTGGGGGCACAGCGG |
| | | CCCTGGGCTGCCTGGTCAAGGACTACTTCCCCGAAC |
| | | CGGTGACGGTGTCGTGGAACTCAGGCGCCCTGACCA |
| | | GCGGCGTGCACACCTTCCCGGCTGTCCTACAGTCCT |
| | | CAGGACTCTACTCCCTCAGCAGCGTGGTGACCGTGC |
| | | CCTCCAGCAGCTTGGGCACCCAGACCTACATCTGCA |
| | | ACGTGAATCACAAGCCCAGCAACACCAAGGTGGAC |
| | | AAGAAAGTTGAGCCAAAGTCCTGTGACAAGACCCA |
| | | CACATGCCCCCCTTGTCCTGCTCCACCAGCTGCAGG |
| | | ACCAAGCGTGTTCCTGTTTCCACCCAAGCCCAAGGA |
| | | TACCCTGATGATCTCTCGGACCCCAGAGGTGACATG |
| | | CGTGGTGGTGGATGTGAGCCACGAGGACCCCGAGG |
| | | TGAAGTTCAACTGGTATGTGGACGGCGTGGAGGTGC |
| | | ACAATGCTAAGACCAAGCCCAGGGAGGAGCAGTAC |
| | | AACTCCACCTATAGAGTGGTGTCTGTGCTGACAGTG |
| | | CTGCACCAGGATTGGCTGAACGGCAAGGAGTATAA |
| | | GTGCAAGGTGTCCAATAAGGCCCTGGCCGCTCCTAT |
| | | CGAGAAGACCATCTCTAAGGCCAAGGGCCAGCCCA |
| | | GAGAGCCTCAGGTGTACACACTGCCTCCATCCCGGG |
| | | AAGAGATGACCAAGAACCAGGTGTCTCTGACATGTC |
| | | TGGTCAAGGGCTTCTATCCCTCTGACATCGCCGTGG |
| | | AGTGGGAGAGCAATGGCCAGCCTGAGAACAATTAC |
| | | AAGACCACACCCCCTGTGCTGGATTCCGACGGCTCT |
| | | TTCTTTCTGTATAGCAAGCTGACCGTGGACAAGTCC |
| | | CGGTGGCAGCAGGGCAACGTGTTCAGCTGTTCCGTG |
| | | ATGCACGAAGCTCTGCATAATCACTATACTCAGAAA |
| | | TCCCTGTCACTGTCACCTGGTAAAGGTGGAGGCGGT |
| | | TCAGAGGTCCAGTTACTTGAGAGTGGTGGAGGTCTG |
| | | GTCCAACCAGGAGGTTCGCTGCGTTTATCCTGCGCC |
| | | GCGTCTGGATTCACGCTTTCCGCCGAAGACGTGGGT |
| | | TGGGTGCGTCAAGCGCCGGGGAAAGGACTGGAATG |
| | | GGTCTCCGCCATCTTGGATCATGGTGGTTCGACATA |
| | | CTATGCGGAAAGTGTCAAAGGGCGCTTTACGATCTC |
| | | GCGCGATAACGCAAAAAATACTCTTTACCTTCAAAT |
| | | GTCTAGCCTTCGTGCTGAGGACACTGCGGTGTACTA |
| | | CTGCGCCCGCGTTGTCTACCATGCTGGTGGTGGCGT |
| | | CACCTATGATTATCGGGGACAGGGCACCCAAGTTAC |
| | | AGTCTCATCG |
| SEQ ID NO: | Format A | QVQLVQSGAEVKKPGASVKVSCKASGYIFTSYYLHW |
| 267 | BGA-3953 | VRQAPGQGLEWIGYINPQTGKTSYAQKFQGRVTMTR |
| | AA | DTSTSTVYMELSSLRSEDTAVYYCAREYGNYNYPLDY |
| | | WGQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPPAAGPSVFLFPPKPKDTLMISRTPEV |
| | | TCVVVDVSHEDPEVKFNWYVDGVEVHNAKTKPREEQ |
| | | YNSTYRVVSVLTVLHQDWLNGKEYKCKVSNKALAAP |
| | | IEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSL |
| | | SPGKGGGGSEVQLLESGGGLVQPGGSLRLSCAASGFT |
| | | VSAEDVGWVRQAPGKGLEWVSAILDSGGSTYYAESV |
| | | KGRFTISRDNAKNTLYLQMSSLRAEDTAVYYCARIVY |
| | | HAGGGVTFDTRGQGTQVTVSS |
| SEQ ID NO: | Format A | CAGGTGCAGCTGGTGCAGAGCGGCGCGGAAGTGAA |
| 268 | BGA-3953 | AAAACCGGGCGCGAGCGTGAAAGTGAGCTGCAAAG |
| | DNA | CGAGCGGCTATATTTTTACCAGCTATTACCTGCATTG |
| | | GGTGCGCCAGGCGCCGGGCCAGGGCCTGGAATGGA |
| | | TTGGCTATATTAACCCGCAGACCGGCAAGACCAGCT |
| | | ATGCCCAGAAATTTCAGGGCCGCGTGACCATGACCC |
| | | GCGATACCAGCACCAGCACCGTGTATATGGAACTGA |
| | | GCAGCCTGCGCAGCGAAGATACCGCGGTGTATTATT |
| | | GCGCGCGCGAATATGGCAACTATAACTATCCGCTGG |
| | | ATTATTGGGGCCAGGGCACCCTGGTGACCGTGAGCA |
| | | GCGCTAGCACCAAGGGGCCCTCGGTCTTCCCCCTGG |
| | | CACCCTCCTCCAAGAGTACTTCTGGGGGCACAGCGG |
| | | CCCTGGGCTGCCTGGTCAAGGACTACTTCCCCGAAC |
| | | CGGTGACGGTGTCGTGGAACTCAGGCGCCCTGACCA |
| | | GCGGCGTGCACACCTTCCCGGCTGTCCTACAGTCCT |
| | | CAGGACTCTACTCCCTCAGCAGCGTGGTGACCGTGC |
| | | CCTCCAGCAGCTTGGGCACCCAGACCTACATCTGCA |
| | | ACGTGAATCACAAGCCCAGCAACACCAAGGTGGAC |
| | | AAGAAAGTTGAGCCAAAGTCCTGTGACAAGACCCA |
| | | CACATGCCCCCCTTGTCCTGCTCCACCAGCTGCAGG |
| | | ACCAAGCGTGTTCCTGTTTCCACCCAAGCCCAAGGA |
| | | TACCCTGATGATCTCTCGGACCCCAGAGGTGACATG |
| | | CGTGGTGGTGGATGTGAGCCACGAGGACCCCGAGG |
| | | TGAAGTTCAACTGGTATGTGGACGGCGTGGAGGTGC |
| | | ACAATGCTAAGACCAAGCCCAGGGAGGAGCAGTAC |
| | | AACTCCACCTATAGAGTGGTGTCTGTGCTGACAGTG |
| | | CTGCACCAGGATTGGCTGAACGGCAAGGAGTATAA |
| | | GTGCAAGGTGTCCAATAAGGCCCTGGCCGCTCCTAT |
| | | CGAGAAGACCATCTCTAAGGCCAAGGGCCAGCCCA |
| | | GAGAGCCTCAGGTGTACACACTGCCTCCATCCCGGG |
| | | AAGAGATGACCAAGAACCAGGTGTCTCTGACATGTC |
| | | TGGTCAAGGGCTTCTATCCCTCTGACATCGCCGTGG |
| | | AGTGGGAGAGCAATGGCCAGCCTGAGAACAATTAC |
| | | AAGACCACACCCCCTGTGCTGGATTCCGACGGCTCT |
| | | TTCTTTCTGTATAGCAAGCTGACCGTGGACAAGTCC |
| | | CGGTGGCAGCAGGGCAACGTGTTCAGCTGTTCCGTG |
| | | ATGCACGAAGCTCTGCATAATCACTATACTCAGAAA |
| | | TCCCTGTCACTGTCACCTGGTAAAGGTGGAGGCGGT |
| | | TCAGAGGTCCAGTTACTTGAGAGTGGTGGAGGTCTG |
| | | GTCCAACCAGGAGGTTCGCTGCGTTTATCCTGCGCC |
| | | GCGTCTGGATTCACGGTTTCCGCCGAAGACGTGGGT |
| | | TGGGTGCGTCAAGCGCCGGGGAAAGGACTGGAATG |
| | | GGTCTCCGCCATCTTGGATtcGGGTGGTTCGACATAC |
| | | TATGCGGAAAGTGTCAAAGGGCGCTTTACGATCTCG |
| | | CGCGATAACGCAAAAAATACTCTTTACCTTCAAATG |
| | | TCTAGCCTTCGTGCTGAGGACACTGCGGTGTACTAC |
| | | TGCGCCCGCATTGTCTACCATGCTGGTGGTGGCGTC |
| | | ACCTTTGATACTCGGGGACAGGGCACCCAAGTTACA |
| | | GTCTCATCG |
| SEQ ID NO: | Format A | QVQLVQSGAEVKKPGASVKVSCKASGYIFTSYYLHW |
| 269 | BGA-5385 | VRQAPGQGLEWIGYINPQTGKTSYAQKFQGRVTMTR |
| | AA | DTSTSTVYMELSSLRSEDTAVYYCAREYGNYNYPLDY |
| | | WGQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPPAAGPSVFLFPPKPKDTLMISRTPEV |
| | | TCVVVDVSHEDPEVKFNWYVDGVEVHNAKTKPREEQ |
| | | YNSTYRVVSVLTVLHQDWLNGKEYKCKVSNKALAAP |
| | | IEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSL |
| | | SPGKGGGGSEVQLLESGGGLVQPGGSLRLSCAASGFT |
| | | LSAEDVGWVRQAPGKGLEWVSAILDHGGSTYYAESV |
| | | KGRFTISRDNAKNTLYLQMSSLRAEDTAVYYCARVIY |
| | | HAGGGVTFDTRGQGTQVTVSS |
| SEQ ID NO: | Format A | CAGGTGCAGCTGGTGCAGAGCGGCGCGGAAGTGAA |
| 270 | BGA-5385 | AAAACCGGGCGCGAGCGTGAAAGTGAGCTGCAAAG |
| | DNA | CGAGCGGCTATATTTTTACCAGCTATTACCTGCATTG |
| | | GGTGCGCCAGGCGCCGGGCCAGGGCCTGGAATGGA |
| | | TTGGCTATATTAACCCGCAGACCGGCAAGACCAGCT |
| | | ATGCCCAGAAATTTCAGGGCCGCGTGACCATGACCC |
| | | GCGATACCAGCACCAGCACCGTGTATATGGAACTGA |
| | | GCAGCCTGCGCAGCGAAGATACCGCGGTGTATTATT |
| | | GCGCGCGCGAATATGGCAACTATAACTATCCGCTGG |
| | | ATTATTGGGGCCAGGGCACCCTGGTGACCGTGAGCA |
| | | GCGCTAGCACCAAGGGGCCCTCGGTCTTCCCCCTGG |
| | | CACCCTCCTCCAAGAGTACTTCTGGGGGCACAGCGG |
| | | CCCTGGGCTGCCTGGTCAAGGACTACTTCCCCGAAC |
| | | CGGTGACGGTGTCGTGGAACTCAGGCGCCCTGACCA |
| | | GCGGCGTGCACACCTTCCCGGCTGTCCTACAGTCCT |
| | | CAGGACTCTACTCCCTCAGCAGCGTGGTGACCGTGC |
| | | CCTCCAGCAGCTTGGGCACCCAGACCTACATCTGCA |
| | | ACGTGAATCACAAGCCCAGCAACACCAAGGTGGAC |
| | | AAGAAAGTTGAGCCAAAGTCCTGTGACAAGACCCA |
| | | CACATGCCCCCCTTGTCCTGCTCCACCAGCTGCAGG |
| | | ACCAAGCGTGTTCCTGTTTCCACCCAAGCCCAAGGA |
| | | TACCCTGATGATCTCTCGGACCCCAGAGGTGACATG |
| | | CGTGGTGGTGGATGTGAGCCACGAGGACCCCGAGG |
| | | TGAAGTTCAACTGGTATGTGGACGGCGTGGAGGTGC |
| | | ACAATGCTAAGACCAAGCCCAGGGAGGAGCAGTAC |
| | | AACTCCACCTATAGAGTGGTGTCTGTGCTGACAGTG |
| | | CTGCACCAGGATTGGCTGAACGGCAAGGAGTATAA |
| | | GTGCAAGGTGTCCAATAAGGCCCTGGCCGCTCCTAT |
| | | CGAGAAGACCATCTCTAAGGCCAAGGGCCAGCCCA |
| | | GAGAGCCTCAGGTGTACACACTGCCTCCATCCCGGG |
| | | AAGAGATGACCAAGAACCAGGTGTCTCTGACATGTC |
| | | TGGTCAAGGGCTTCTATCCCTCTGACATCGCCGTGG |
| | | AGTGGGAGAGCAATGGCCAGCCTGAGAACAATTAC |
| | | AAGACCACACCCCCTGTGCTGGATTCCGACGGCTCT |
| | | TTCTTTCTGTATAGCAAGCTGACCGTGGACAAGTCC |
| | | CGGTGGCAGCAGGGCAACGTGTTCAGCTGTTCCGTG |
| | | ATGCACGAAGCTCTGCATAATCACTATACTCAGAAA |
| | | TCCCTGTCACTGTCACCTGGTAAAGGTGGAGGCGGT |
| | | TCAGAGGTCCAGTTACTTGAGAGTGGTGGAGGTCTG |
| | | GTCCAACCAGGAGGTTCGCTGCGTTTATCCTGCGCC |
| | | GCGTCTGGATTCACGCTTTCCGCCGAAGACGTGGGT |
| | | TGGGTGCGTCAAGCGCCGGGGAAAGGACTGGAATG |
| | | GGTCTCCGCCATCTTGGATCATGGTGGTTCGACATA |
| | | CTATGCGGAAAGTGTCAAAGGGCGCTTTACGATCTC |
| | | GCGCGATAACGCAAAAAATACTCTTTACCTTCAAAT |
| | | GTCTAGCCTTCGTGCTGAGGACACTGCGGTGTACTA |
| | | CTGCGCCCGCGTTATCTACCATGCTGGTGGTGGCGT |
| | | CACCTTTGATACTCGGGGACAGGGCACCCAAGTTAC |
| | | AGTCTCATCG |
| SEQ ID NO: | Format A | QVQLVQSGAEVKKPGASVKVSCKASGYIFTSYYLHW |
| 271 | BGA-9468 | VRQAPGQGLEWIGYINPQTGKTSYAQKFQGRVTMTR |
| | AA | DTSTSTVYMELSSLRSEDTAVYYCAREYGNYNYPLDY |
| | | WGQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPPAAGPSVFLFPPKPKDTLMISRTPEV |
| | | TCVVVDVSHEDPEVKFNWYVDGVEVHNAKTKPREEQ |
| | | YNSTYRVVSVLTVLHQDWLNGKEYKCKVSNKALAAP |
| | | IEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSL |
| | | SPGKGGGGSEVQLLESGGGLVQPGGSLRLSCAASGFT |
| | | VSAEDVGWVRQAPGKGLEWVSAILDSGGSTYYAESV |
| | | KGRFTISRDNAKNTLYLQMSSLRAEDTAVYYCARVVY |
| | | HSGGGVTFDLRGQGTQVTVSS |
| SEQ ID NO: | Format A | CAGGTGCAGCTGGTGCAGAGCGGCGCGGAAGTGAA |
| 272 | BGA-9468 | AAAACCGGGCGCGAGCGTGAAAGTGAGCTGCAAAG |
| | DNA | CGAGCGGCTATATTTTTACCAGCTATTACCTGCATTG |
| | | GGTGCGCCAGGCGCCGGGCCAGGGCCTGGAATGGA |
| | | TTGGCTATATTAACCCGCAGACCGGCAAGACCAGCT |
| | | ATGCCCAGAAATTTCAGGGCCGCGTGACCATGACCC |
| | | GCGATACCAGCACCAGCACCGTGTATATGGAACTGA |
| | | GCAGCCTGCGCAGCGAAGATACCGCGGTGTATTATT |
| | | GCGCGCGCGAATATGGCAACTATAACTATCCGCTGG |
| | | ATTATTGGGGCCAGGGCACCCTGGTGACCGTGAGCA |
| | | GCGCTAGCACCAAGGGGCCCTCGGTCTTCCCCCTGG |
| | | CACCCTCCTCCAAGAGTACTTCTGGGGGCACAGCGG |
| | | CCCTGGGCTGCCTGGTCAAGGACTACTTCCCCGAAC |
| | | CGGTGACGGTGTCGTGGAACTCAGGCGCCCTGACCA |
| | | GCGGCGTGCACACCTTCCCGGCTGTCCTACAGTCCT |
| | | CAGGACTCTACTCCCTCAGCAGCGTGGTGACCGTGC |
| | | CCTCCAGCAGCTTGGGCACCCAGACCTACATCTGCA |
| | | ACGTGAATCACAAGCCCAGCAACACCAAGGTGGAC |
| | | AAGAAAGTTGAGCCAAAGTCCTGTGACAAGACCCA |
| | | CACATGCCCCCCTTGTCCTGCTCCACCAGCTGCAGG |
| | | ACCAAGCGTGTTCCTGTTTCCACCCAAGCCCAAGGA |
| | | TACCCTGATGATCTCTCGGACCCCAGAGGTGACATG |
| | | CGTGGTGGTGGATGTGAGCCACGAGGACCCCGAGG |
| | | TGAAGTTCAACTGGTATGTGGACGGCGTGGAGGTGC |
| | | ACAATGCTAAGACCAAGCCCAGGGAGGAGCAGTAC |
| | | AACTCCACCTATAGAGTGGTGTCTGTGCTGACAGTG |
| | | CTGCACCAGGATTGGCTGAACGGCAAGGAGTATAA |
| | | GTGCAAGGTGTCCAATAAGGCCCTGGCCGCTCCTAT |
| | | CGAGAAGACCATCTCTAAGGCCAAGGGCCAGCCCA |
| | | GAGAGCCTCAGGTGTACACACTGCCTCCATCCCGGG |
| | | AAGAGATGACCAAGAACCAGGTGTCTCTGACATGTC |
| | | TGGTCAAGGGCTTCTATCCCTCTGACATCGCCGTGG |
| | | AGTGGGAGAGCAATGGCCAGCCTGAGAACAATTAC |
| | | AAGACCACACCCCCTGTGCTGGATTCCGACGGCTCT |
| | | TTCTTTCTGTATAGCAAGCTGACCGTGGACAAGTCC |
| | | CGGTGGCAGCAGGGCAACGTGTTCAGCTGTTCCGTG |
| | | ATGCACGAAGCTCTGCATAATCACTATACTCAGAAA |
| | | TCCCTGTCACTGTCACCTGGTAAAGGTGGAGGCGGT |
| | | TCAGAGGTCCAGTTACTTGAGAGTGGTGGAGGTCTG |
| | | GTCCAACCAGGAGGTTCGCTGCGTTTATCCTGCGCC |
| | | GCGTCTGGATTCACGGTTTCCGCCGAAGACGTGGGT |
| | | TGGGTGCGTCAAGCGCCGGGGAAAGGACTGGAATG |
| | | GGTCTCCGCCATCTTGGATTCGGGTGGTTCGACATA |
| | | CTATGCGGAAAGTGTCAAAGGGCGCTTTACGATCTC |
| | | GCGCGATAACGCAAAAAATACTCTTTACCTTCAAAT |
| | | GTCTAGCCTTCGTGCTGAGGACACTGCGGTGTACTA |
| | | CTGCGCCCGCGTTGTCTACCATTCTGGTGGTGGCGT |
| | | CACCTTTGATCTTCGGGGACAGGGCACCCAAGTTAC |
| | | AGTCTCATCG |
| SEQ ID NO: | Format A | QVQLVQSGAEVKKPGASVKVSCKASGYIFTSYYLHW |
| 273 | BGA-3285 | VRQAPGQGLEWIGYINPQTGKTSYAQKFQGRVTMTR |
| | AA | DTSTSTVYMELSSLRSEDTAVYYCAREYGNYNYPLDY |
| | | WGQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPPAAGPSVFLFPPKPKDTLMISRTPEV |
| | | TCVVVDVSHEDPEVKFNWYVDGVEVHNAKTKPREEQ |
| | | YNSTYRVVSVLTVLHQDWLNGKEYKCKVSNKALAAP |
| | | IEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSL |
| | | SPGKGGGGSEVQLLESGGGLVQPGGSLRLSCAASGFTI |
| | | SAEDVGWVRQAPGKGLEWVSAILDFGGSTYYAESVK |
| | | GRFTISRDNAKNTLYLQMSSLRAEDTAVYYCARVVYH |
| | | AGGGVTFDTRGQGTQVTVSS |
| SEQ ID NO: | Format A | CAGGTGCAGCTGGTGCAGAGCGGCGCGGAAGTGAA |
| 274 | BGA-3285 | AAAACCGGGCGCGAGCGTGAAAGTGAGCTGCAAAG |
| | DNA | CGAGCGGCTATATTTTTACCAGCTATTACCTGCATTG |
| | | GGTGCGCCAGGCGCCGGGCCAGGGCCTGGAATGGA |
| | | TTGGCTATATTAACCCGCAGACCGGCAAGACCAGCT |
| | | ATGCCCAGAAATTTCAGGGCCGCGTGACCATGACCC |
| | | GCGATACCAGCACCAGCACCGTGTATATGGAACTGA |
| | | GCAGCCTGCGCAGCGAAGATACCGCGGTGTATTATT |
| | | GCGCGCGCGAATATGGCAACTATAACTATCCGCTGG |
| | | ATTATTGGGGCCAGGGCACCCTGGTGACCGTGAGCA |
| | | GCGCTAGCACCAAGGGGCCCTCGGTCTTCCCCCTGG |
| | | CACCCTCCTCCAAGAGTACTTCTGGGGGCACAGCGG |
| | | CCCTGGGCTGCCTGGTCAAGGACTACTTCCCCGAAC |
| | | CGGTGACGGTGTCGTGGAACTCAGGCGCCCTGACCA |
| | | GCGGCGTGCACACCTTCCCGGCTGTCCTACAGTCCT |
| | | CAGGACTCTACTCCCTCAGCAGCGTGGTGACCGTGC |
| | | CCTCCAGCAGCTTGGGCACCCAGACCTACATCTGCA |
| | | ACGTGAATCACAAGCCCAGCAACACCAAGGTGGAC |
| | | AAGAAAGTTGAGCCAAAGTCCTGTGACAAGACCCA |
| | | CACATGCCCCCCTTGTCCTGCTCCACCAGCTGCAGG |
| | | ACCAAGCGTGTTCCTGTTTCCACCCAAGCCCAAGGA |
| | | TACCCTGATGATCTCTCGGACCCCAGAGGTGACATG |
| | | CGTGGTGGTGGATGTGAGCCACGAGGACCCCGAGG |
| | | TGAAGTTCAACTGGTATGTGGACGGCGTGGAGGTGC |
| | | ACAATGCTAAGACCAAGCCCAGGGAGGAGCAGTAC |
| | | AACTCCACCTATAGAGTGGTGTCTGTGCTGACAGTG |
| | | CTGCACCAGGATTGGCTGAACGGCAAGGAGTATAA |
| | | GTGCAAGGTGTCCAATAAGGCCCTGGCCGCTCCTAT |
| | | CGAGAAGACCATCTCTAAGGCCAAGGGCCAGCCCA |
| | | GAGAGCCTCAGGTGTACACACTGCCTCCATCCCGGG |
| | | AAGAGATGACCAAGAACCAGGTGTCTCTGACATGTC |
| | | TGGTCAAGGGCTTCTATCCCTCTGACATCGCCGTGG |
| | | AGTGGGAGAGCAATGGCCAGCCTGAGAACAATTAC |
| | | AAGACCACACCCCCTGTGCTGGATTCCGACGGCTCT |
| | | TTCTTTCTGTATAGCAAGCTGACCGTGGACAAGTCC |
| | | CGGTGGCAGCAGGGCAACGTGTTCAGCTGTTCCGTG |
| | | ATGCACGAAGCTCTGCATAATCACTATACTCAGAAA |
| | | TCCCTGTCACTGTCACCTGGTAAAGGTGGAGGCGGT |
| | | TCAGAGGTCCAGTTACTTGAGAGTGGTGGAGGTCTG |
| | | GTCCAACCAGGAGGTTCGCTGCGTTTATCCTGCGCC |
| | | GCGTCTGGATTCACGATTTCCGCCGAAGACGTGGGT |
| | | TGGGTGCGTCAAGCGCCGGGGAAAGGACTGGAATG |
| | | GGTCTCCGCCATCTTGGATTTTGGTGGTTCGACATAC |
| | | TATGCGGAAAGTGTCAAAGGGCGCTTTACGATCTCG |
| | | CGCGATAACGCAAAAAATACTCTTTACCTTCAAATG |
| | | TCTAGCCTTCGTGCTGAGGACACTGCGGTGTACTAC |
| | | TGCGCCCGCGTTGTCTACCATGCTGGTGGTGGCGTC |
| | | ACCTTTGATACTCGGGGACAGGGCACCCAAGTTACA |
| | | GTCTCATCG |
| SEQ ID NO: | Format A | QVQLVQSGAEVKKPGASVKVSCKASGYIFTSYYLHW |
| 275 | BGA-9442 | VRQAPGQGLEWIGYINPQTGKTSYAQKFQGRVTMTR |
| | AA | DTSTSTVYMELSSLRSEDTAVYYCAREYGNYNYPLDY |
| | | WGQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPPAAGPSVFLFPPKPKDTLMISRTPEV |
| | | TCVVVDVSHEDPEVKFNWYVDGVEVHNAKTKPREEQ |
| | | YNSTYRVVSVLTVLHQDWLNGKEYKCKVSNKALAAP |
| | | IEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSL |
| | | SPGKGGGGSEVQLLESGGGLVQPGGSLRLSCAASGFTI |
| | | SAEDVGWVRQAPGKGLEWVSAILDSGGSTYYAESVK |
| | | GRFTISRDNAKNTLYLQMSSLRAEDTAVYYCARYVYH |
| | | AGGGVTFDLRGQGTQVTVSS |
| SEQ ID NO: | Format A | CAGGTGCAGCTGGTGCAGAGCGGCGCGGAAGTGAA |
| 276 | BGA-9442 | AAAACCGGGCGCGAGCGTGAAAGTGAGCTGCAAAG |
| | DNA | CGAGCGGCTATATTTTTACCAGCTATTACCTGCATTG |
| | | GGTGCGCCAGGCGCCGGGCCAGGGCCTGGAATGGA |
| | | TTGGCTATATTAACCCGCAGACCGGCAAGACCAGCT |
| | | ATGCCCAGAAATTTCAGGGCCGCGTGACCATGACCC |
| | | GCGATACCAGCACCAGCACCGTGTATATGGAACTGA |
| | | GCAGCCTGCGCAGCGAAGATACCGCGGTGTATTATT |
| | | GCGCGCGCGAATATGGCAACTATAACTATCCGCTGG |
| | | ATTATTGGGGCCAGGGCACCCTGGTGACCGTGAGCA |
| | | GCGCTAGCACCAAGGGGCCCTCGGTCTTCCCCCTGG |
| | | CACCCTCCTCCAAGAGTACTTCTGGGGGCACAGCGG |
| | | CCCTGGGCTGCCTGGTCAAGGACTACTTCCCCGAAC |
| | | CGGTGACGGTGTCGTGGAACTCAGGCGCCCTGACCA |
| | | GCGGCGTGCACACCTTCCCGGCTGTCCTACAGTCCT |
| | | CAGGACTCTACTCCCTCAGCAGCGTGGTGACCGTGC |
| | | CCTCCAGCAGCTTGGGCACCCAGACCTACATCTGCA |
| | | ACGTGAATCACAAGCCCAGCAACACCAAGGTGGAC |
| | | AAGAAAGTTGAGCCAAAGTCCTGTGACAAGACCCA |
| | | CACATGCCCCCCTTGTCCTGCTCCACCAGCTGCAGG |
| | | ACCAAGCGTGTTCCTGTTTCCACCCAAGCCCAAGGA |
| | | TACCCTGATGATCTCTCGGACCCCAGAGGTGACATG |
| | | CGTGGTGGTGGATGTGAGCCACGAGGACCCCGAGG |
| | | TGAAGTTCAACTGGTATGTGGACGGCGTGGAGGTGC |
| | | ACAATGCTAAGACCAAGCCCAGGGAGGAGCAGTAC |
| | | AACTCCACCTATAGAGTGGTGTCTGTGCTGACAGTG |
| | | CTGCACCAGGATTGGCTGAACGGCAAGGAGTATAA |
| | | GTGCAAGGTGTCCAATAAGGCCCTGGCCGCTCCTAT |
| | | CGAGAAGACCATCTCTAAGGCCAAGGGCCAGCCCA |
| | | GAGAGCCTCAGGTGTACACACTGCCTCCATCCCGGG |
| | | AAGAGATGACCAAGAACCAGGTGTCTCTGACATGTC |
| | | TGGTCAAGGGCTTCTATCCCTCTGACATCGCCGTGG |
| | | AGTGGGAGAGCAATGGCCAGCCTGAGAACAATTAC |
| | | AAGACCACACCCCCTGTGCTGGATTCCGACGGCTCT |
| | | TTCTTTCTGTATAGCAAGCTGACCGTGGACAAGTCC |
| | | CGGTGGCAGCAGGGCAACGTGTTCAGCTGTTCCGTG |
| | | ATGCACGAAGCTCTGCATAATCACTATACTCAGAAA |
| | | TCCCTGTCACTGTCACCTGGTAAAGGTGGAGGCGGT |
| | | TCAGAGGTCCAGTTACTTGAGAGTGGTGGAGGTCTG |
| | | GTCCAACCAGGAGGTTCGCTGCGTTTATCCTGCGCC |
| | | GCGTCTGGATTCACGATTTCCGCCGAAGACGTGGGT |
| | | TGGGTGCGTCAAGCGCCGGGGAAAGGACTGGAATG |
| | | GGTCTCCGCCATCTTGGATTCTGGTGGTTCGACATA |
| | | CTATGCGGAAAGTGTCAAAGGGCGCTTTACGATCTC |
| | | GCGCGATAACGCAAAAAATACTCTTTACCTTCAAAT |
| | | GTCTAGCCTTCGTGCTGAGGACACTGCGGTGTACTA |
| | | CTGCGCCCGCTATGTCTACCATGCTGGTGGTGGCGT |
| | | CACCTTTGATCTTCGGGGACAGGGCACCCAAGTTAC |
| | | AGTCTCATCG |
| SEQ ID NO: | Format A | QVQLVQSGAEVKKPGASVKVSCKASGYIFTSYYLHW |
| 277 | BGA-7746 | VRQAPGQGLEWIGYINPQTGKTSYAQKFQGRVTMTR |
| | AA | DTSTSTVYMELSSLRSEDTAVYYCAREYGNYNYPLDY |
| | | WGQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPPAAGPSVFLFPPKPKDTLMISRTPEV |
| | | TCVVVDVSHEDPEVKFNWYVDGVEVHNAKTKPREEQ |
| | | YNSTYRVVSVLTVLHQDWLNGKEYKCKVSNKALAAP |
| | | IEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSL |
| | | SPGKGGGGSEVQLLESGGGLVQPGGSLRLSCAASGFT |
| | | VSAEDVGWVRQAPGKGLEWVSAILDFGGSTYYAESV |
| | | KGRFTISRDNAKNTLYLQMSSLRAEDTAVYYCARVVY |
| | | HAGGGVTFDTRGQGTQVTVSS |
| SEQ ID NO: | Format A | CAGGTGCAGCTGGTGCAGAGCGGCGCGGAAGTGAA |
| 278 | BGA-7746 | AAAACCGGGCGCGAGCGTGAAAGTGAGCTGCAAAG |
| | DNA | CGAGCGGCTATATTTTTACCAGCTATTACCTGCATTG |
| | | GGTGCGCCAGGCGCCGGGCCAGGGCCTGGAATGGA |
| | | TTGGCTATATTAACCCGCAGACCGGCAAGACCAGCT |
| | | ATGCCCAGAAATTTCAGGGCCGCGTGACCATGACCC |
| | | GCGATACCAGCACCAGCACCGTGTATATGGAACTGA |
| | | GCAGCCTGCGCAGCGAAGATACCGCGGTGTATTATT |
| | | GCGCGCGCGAATATGGCAACTATAACTATCCGCTGG |
| | | ATTATTGGGGCCAGGGCACCCTGGTGACCGTGAGCA |
| | | GCGCTAGCACCAAGGGGCCCTCGGTCTTCCCCCTGG |
| | | CACCCTCCTCCAAGAGTACTTCTGGGGGCACAGCGG |
| | | CCCTGGGCTGCCTGGTCAAGGACTACTTCCCCGAAC |
| | | CGGTGACGGTGTCGTGGAACTCAGGCGCCCTGACCA |
| | | GCGGCGTGCACACCTTCCCGGCTGTCCTACAGTCCT |
| | | CAGGACTCTACTCCCTCAGCAGCGTGGTGACCGTGC |
| | | CCTCCAGCAGCTTGGGCACCCAGACCTACATCTGCA |
| | | ACGTGAATCACAAGCCCAGCAACACCAAGGTGGAC |
| | | AAGAAAGTTGAGCCAAAGTCCTGTGACAAGACCCA |
| | | CACATGCCCCCCTTGTCCTGCTCCACCAGCTGCAGG |
| | | ACCAAGCGTGTTCCTGTTTCCACCCAAGCCCAAGGA |
| | | TACCCTGATGATCTCTCGGACCCCAGAGGTGACATG |
| | | CGTGGTGGTGGATGTGAGCCACGAGGACCCCGAGG |
| | | TGAAGTTCAACTGGTATGTGGACGGCGTGGAGGTGC |
| | | ACAATGCTAAGACCAAGCCCAGGGAGGAGCAGTAC |
| | | AACTCCACCTATAGAGTGGTGTCTGTGCTGACAGTG |
| | | CTGCACCAGGATTGGCTGAACGGCAAGGAGTATAA |
| | | GTGCAAGGTGTCCAATAAGGCCCTGGCCGCTCCTAT |
| | | CGAGAAGACCATCTCTAAGGCCAAGGGCCAGCCCA |
| | | GAGAGCCTCAGGTGTACACACTGCCTCCATCCCGGG |
| | | AAGAGATGACCAAGAACCAGGTGTCTCTGACATGTC |
| | | TGGTCAAGGGCTTCTATCCCTCTGACATCGCCGTGG |
| | | AGTGGGAGAGCAATGGCCAGCCTGAGAACAATTAC |
| | | AAGACCACACCCCCTGTGCTGGATTCCGACGGCTCT |
| | | TTCTTTCTGTATAGCAAGCTGACCGTGGACAAGTCC |
| | | CGGTGGCAGCAGGGCAACGTGTTCAGCTGTTCCGTG |
| | | ATGCACGAAGCTCTGCATAATCACTATACTCAGAAA |
| | | TCCCTGTCACTGTCACCTGGTAAAGGTGGAGGCGGT |
| | | TCAGAGGTCCAGTTACTTGAGAGTGGTGGAGGTCTG |
| | | GTCCAACCAGGAGGTTCGCTGCGTTTATCCTGCGCC |
| | | GCGTCTGGATTCACGGTTTCCGCCGAAGACGTGGGT |
| | | TGGGTGCGTCAAGCGCCGGGGAAAGGACTGGAATG |
| | | GGTCTCCGCCATCTTGGATTTTGGTGGTTCGACATAC |
| | | TATGCAGAAAGTGTCAAAGGGCGCTTTACGATCTCG |
| | | CGCGATAACGCAAAAAATACTCTTTACCTTCAAATG |
| | | TCTAGCCTTCGTGCTGAGGACACTGCGGTGTACTAC |
| | | TGCGCCCGCGTCGTCTACCATGCTGGTGGTGGCGTC |
| | | ACCTTTGATACGCGGGGACAGGGCACCCAAGTTACA |
| | | GTCTCATCG |
| SEQ ID NO: | Format A | QVQLVQSGAEVKKPGASVKVSCKASGYIFTSYYLHW |
| 279 | BGA-8425 | VRQAPGQGLEWIGYINPQTGKTSYAQKFQGRVTMTR |
| | AA | DTSTSTVYMELSSLRSEDTAVYYCAREYGNYNYPLDY |
| | | WGQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPPAAGPSVFLFPPKPKDTLMISRTPEV |
| | | TCVVVDVSHEDPEVKFNWYVDGVEVHNAKTKPREEQ |
| | | YNSTYRVVSVLTVLHQDWLNGKEYKCKVSNKALAAP |
| | | IEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSL |
| | | SPGKGGGGSEVQLLESGGGLVQPGGSLRLSCAASGFT |
| | | LSAEDVGWVRQAPGKGLEWVSAILDSGGSTYYAESV |
| | | KGRFTISRDNAKNTLYLQMSSLRAEDTAVYYCARVVY |
| | | HAGGGVTYDYRGQGTQVTVSS |
| SEQ ID NO: | Format A | CAGGTGCAGCTGGTGCAGAGCGGCGCGGAAGTGAA |
| 280 | BGA-8425 | AAAACCGGGCGCGAGCGTGAAAGTGAGCTGCAAAG |
| | DNA | CGAGCGGCTATATTTTTACCAGCTATTACCTGCATTG |
| | | GGTGCGCCAGGCGCCGGGCCAGGGCCTGGAATGGA |
| | | TTGGCTATATTAACCCGCAGACCGGCAAGACCAGCT |
| | | ATGCCCAGAAATTTCAGGGCCGCGTGACCATGACCC |
| | | GCGATACCAGCACCAGCACCGTGTATATGGAACTGA |
| | | GCAGCCTGCGCAGCGAAGATACCGCGGTGTATTATT |
| | | GCGCGCGCGAATATGGCAACTATAACTATCCGCTGG |
| | | ATTATTGGGGCCAGGGCACCCTGGTGACCGTGAGCA |
| | | GCGCTAGCACCAAGGGGCCCTCGGTCTTCCCCCTGG |
| | | CACCCTCCTCCAAGAGTACTTCTGGGGGCACAGCGG |
| | | CCCTGGGCTGCCTGGTCAAGGACTACTTCCCCGAAC |
| | | CGGTGACGGTGTCGTGGAACTCAGGCGCCCTGACCA |
| | | GCGGCGTGCACACCTTCCCGGCTGTCCTACAGTCCT |
| | | CAGGACTCTACTCCCTCAGCAGCGTGGTGACCGTGC |
| | | CCTCCAGCAGCTTGGGCACCCAGACCTACATCTGCA |
| | | ACGTGAATCACAAGCCCAGCAACACCAAGGTGGAC |
| | | AAGAAAGTTGAGCCAAAGTCCTGTGACAAGACCCA |
| | | CACATGCCCCCCTTGTCCTGCTCCACCAGCTGCAGG |
| | | ACCAAGCGTGTTCCTGTTTCCACCCAAGCCCAAGGA |
| | | TACCCTGATGATCTCTCGGACCCCAGAGGTGACATG |
| | | CGTGGTGGTGGATGTGAGCCACGAGGACCCCGAGG |
| | | TGAAGTTCAACTGGTATGTGGACGGCGTGGAGGTGC |
| | | ACAATGCTAAGACCAAGCCCAGGGAGGAGCAGTAC |
| | | AACTCCACCTATAGAGTGGTGTCTGTGCTGACAGTG |
| | | CTGCACCAGGATTGGCTGAACGGCAAGGAGTATAA |
| | | GTGCAAGGTGTCCAATAAGGCCCTGGCCGCTCCTAT |
| | | CGAGAAGACCATCTCTAAGGCCAAGGGCCAGCCCA |
| | | GAGAGCCTCAGGTGTACACACTGCCTCCATCCCGGG |
| | | AAGAGATGACCAAGAACCAGGTGTCTCTGACATGTC |
| | | TGGTCAAGGGCTTCTATCCCTCTGACATCGCCGTGG |
| | | AGTGGGAGAGCAATGGCCAGCCTGAGAACAATTAC |
| | | AAGACCACACCCCCTGTGCTGGATTCCGACGGCTCT |
| | | TTCTTTCTGTATAGCAAGCTGACCGTGGACAAGTCC |
| | | CGGTGGCAGCAGGGCAACGTGTTCAGCTGTTCCGTG |
| | | ATGCACGAAGCTCTGCATAATCACTATACTCAGAAA |
| | | TCCCTGTCACTGTCACCTGGTAAAGGTGGAGGCGGT |
| | | TCAGAGGTCCAGTTACTTGAGAGTGGTGGAGGTCTG |
| | | GTCCAACCAGGAGGTTCGCTGCGTTTATCCTGCGCC |
| | | GCGTCTGGATTCACGCTGTCCGCCGAAGACGTGGGT |
| | | TGGGTGCGTCAAGCGCCGGGGAAAGGACTGGAATG |
| | | GGTCTCCGCCATCTTGGATTCGGGTGGTTCGACATA |
| | | CTATGCGGAAAGTGTCAAAGGGCGCTTTACGATCTC |
| | | GCGCGATAACGCAAAAAATACTCTTTACCTTCAAAT |
| | | GTCTAGCCTTCGTGCTGAGGACACTGCGGTGTACTA |
| | | CTGCGCCCGCGTCGTCTACCATGCTGGTGGTGGCGT |
| | | CACCTATGATTACCGGGGACAGGGCACCCAAGTTAC |
| | | AGTCTCATCG |
| SEQ ID NO: | Format A | QVQLVQSGAEVKKPGASVKVSCKASGYIFTSYYLHW |
| 281 | BGA-3386 | VRQAPGQGLEWIGYINPQTGKTSYAQKFQGRVTMTR |
| | AA | DTSTSTVYMELSSLRSEDTAVYYCAREYGNYNYPLDY |
| | | WGQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPPAAGPSVFLFPPKPKDTLMISRTPEV |
| | | TCVVVDVSHEDPEVKFNWYVDGVEVHNAKTKPREEQ |
| | | YNSTYRVVSVLTVLHQDWLNGKEYKCKVSNKALAAP |
| | | IEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSL |
| | | SPGKGGGGSEVQLLESGGGLVQPGGSLRLSCAASGFT |
| | | VSAEDVGWVRQAPGKGLEWVSAILDKGGSTYYAESV |
| | | KGRFTISRDNAKNTLYLQMSSLRAEDTAVYYCARIVY |
| | | HAGGGVTFDTWGQGTLVTVSS |
| SEQ ID NO: | Format A | CAGGTGCAGCTGGTGCAGAGCGGCGCGGAAGTGAA |
| 282 | BGA-3386 | AAAACCGGGCGCGAGCGTGAAAGTGAGCTGCAAAG |
| | DNA | CGAGCGGCTATATTTTTACCAGCTATTACCTGCATTG |
| | | GGTGCGCCAGGCGCCGGGCCAGGGCCTGGAATGGA |
| | | TTGGCTATATTAACCCGCAGACCGGCAAGACCAGCT |
| | | ATGCCCAGAAATTTCAGGGCCGCGTGACCATGACCC |
| | | GCGATACCAGCACCAGCACCGTGTATATGGAACTGA |
| | | GCAGCCTGCGCAGCGAAGATACCGCGGTGTATTATT |
| | | GCGCGCGCGAATATGGCAACTATAACTATCCGCTGG |
| | | ATTATTGGGGCCAGGGCACCCTGGTGACCGTGAGCA |
| | | GCGCTAGCACCAAGGGGCCCTCGGTCTTCCCCCTGG |
| | | CACCCTCCTCCAAGAGTACTTCTGGGGGCACAGCGG |
| | | CCCTGGGCTGCCTGGTCAAGGACTACTTCCCCGAAC |
| | | CGGTGACGGTGTCGTGGAACTCAGGCGCCCTGACCA |
| | | GCGGCGTGCACACCTTCCCGGCTGTCCTACAGTCCT |
| | | CAGGACTCTACTCCCTCAGCAGCGTGGTGACCGTGC |
| | | CCTCCAGCAGCTTGGGCACCCAGACCTACATCTGCA |
| | | ACGTGAATCACAAGCCCAGCAACACCAAGGTGGAC |
| | | AAGAAAGTTGAGCCAAAGTCCTGTGACAAGACCCA |
| | | CACATGCCCCCCTTGTCCTGCTCCACCAGCTGCAGG |
| | | ACCAAGCGTGTTCCTGTTTCCACCCAAGCCCAAGGA |
| | | TACCCTGATGATCTCTCGGACCCCAGAGGTGACATG |
| | | CGTGGTGGTGGATGTGAGCCACGAGGACCCCGAGG |
| | | TGAAGTTCAACTGGTATGTGGACGGCGTGGAGGTGC |
| | | ACAATGCTAAGACCAAGCCCAGGGAGGAGCAGTAC |
| | | AACTCCACCTATAGAGTGGTGTCTGTGCTGACAGTG |
| | | CTGCACCAGGATTGGCTGAACGGCAAGGAGTATAA |
| | | GTGCAAGGTGTCCAATAAGGCCCTGGCCGCTCCTAT |
| | | CGAGAAGACCATCTCTAAGGCCAAGGGCCAGCCCA |
| | | GAGAGCCTCAGGTGTACACACTGCCTCCATCCCGGG |
| | | AAGAGATGACCAAGAACCAGGTGTCTCTGACATGTC |
| | | TGGTCAAGGGCTTCTATCCCTCTGACATCGCCGTGG |
| | | AGTGGGAGAGCAATGGCCAGCCTGAGAACAATTAC |
| | | AAGACCACACCCCCTGTGCTGGATTCCGACGGCTCT |
| | | TTCTTTCTGTATAGCAAGCTGACCGTGGACAAGTCC |
| | | CGGTGGCAGCAGGGCAACGTGTTCAGCTGTTCCGTG |
| | | ATGCACGAAGCTCTGCATAATCACTATACTCAGAAA |
| | | TCCCTGTCACTGTCACCTGGTAAAGGTGGAGGCGGT |
| | | TCAGAGGTCCAGTTACTTGAGAGTGGTGGAGGTCTG |
| | | GTCCAACCAGGAGGTTCGCTGCGTTTATCCTGCGCC |
| | | GCGTCTGGATTCACGGTTTCCGCCGAAGACGTGGGT |
| | | TGGGTGCGTCAAGCGCCGGGGAAAGGACTGGAATG |
| | | GGTCTCCGCCATCTTGGATAAGGGTGGTTCGACATA |
| | | CTATGCGGAAAGTGTCAAAGGGCGCTTTACGATCTC |
| | | GCGCGATAACGCAAAAAATACTCTTTACCTTCAAAT |
| | | GTCTAGCCTTCGTGCTGAGGACACTGCGGTGTACTA |
| | | CTGCGCCCGCATTGTCTACCATGCTGGTGGTGGCGT |
| | | CACCTTTGATACTTGGGGACAGGGCACCTTAGTTAC |
| | | AGTCTCATCG |

### Example 22. Binding Profiles of Anti-CD137 Antibody BGA-5623

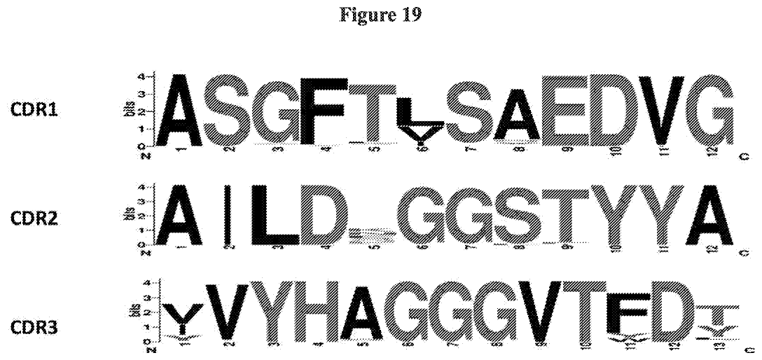

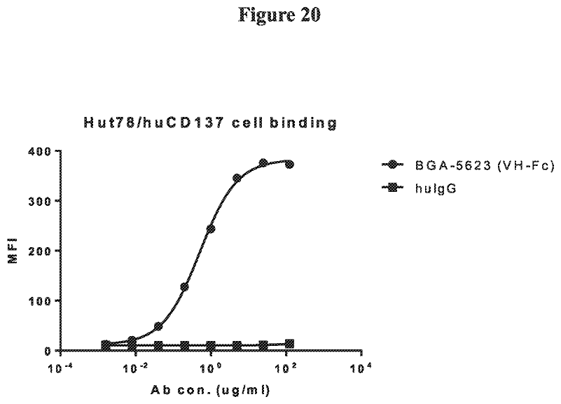

BGA-5623 was generated with human IgG1 Fe fusion and characterized for their binding kinetics by SPR assays using BIAcore™ T-200 (GE Life Sciences). Briefly, anti-human IgG (Fc) antibody was immobilized on an activated CM5 biosensor chip (Cat.: BR100839, GE Life Sciences). The anti-huCD137 domain antibody was flowed through the chip surface and captured by anti-human IgG (Fc) antibody. Then a serial dilution (6.0 nM to 2150 nM) of human CD137 ECD-mIgG2a or cyno CD137 ECD-mIgG2a were flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The result demonstrated that BGA-5623 has higher affinity for cynoCD137 than huCD137, as shown in Table 29 below. To evaluate the binding activity of the anti-huCD137 VH domain antibody to native huCD137 on living cells, Hut78 cells were transfected to over-express human CD137. Live Hut78/huCD137 expressing cells were seeded in 96-well plates and were incubated with a serial dilution of anti-huCD137 VH domain antibodies. Goat anti-Human IgG was used as secondary antibody to detect antibody binding to the cell surface. EC₅₀ values for dose-dependent binding to human native CD137 were determined by fitting the dose-response data to the four-parameter logistic model with GraphPad Prism™. As shown in FIG. 20, BGA-5623 demonstrated specific binding to native CD137 on living cells in a dose-responsive manner with EC50 of 2.97 μg/ml.

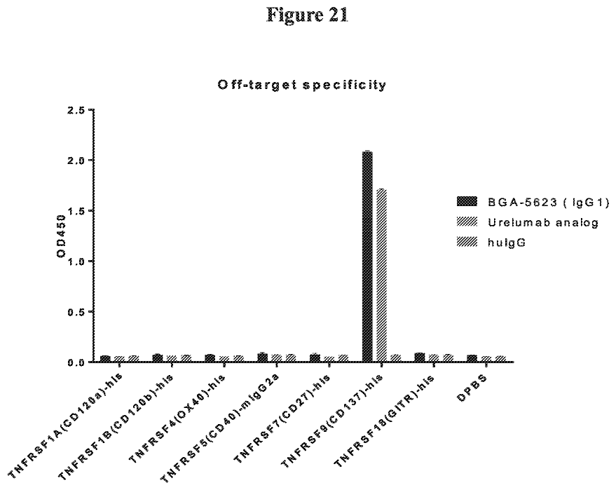

The off-target specificity of BGA-5623 was evaluated via ELISA. TNF receptor family members such as TNFRSF1A(CD12.a) (Cat. No. 10872-H98H, Sino Biological, China), TNFRSF1B(CD120b) (Cat. No. 10417-H08H1, Sino Biological, China), TNFRSF4(OX40) (SEQ ID NO: 167), TNFRSF5(CD4P) (SEQ TD NO: 161), TNFRSF7(CD27) (Cat. No. 10039-H08B1, Sino Biological, China), TNFRSF9(CD137) (SEQ TD NO: 135) and TNFRSF18(GITR) (Cat. No. 13643-H08H, Sino Biological, China) were coated in 96-well plates at a concentration of 10 μg/ml overnight at 4° C. BGA-5623 fused with wild type IgG1 Fc (SEQ ID NO: 283) was added. As shown in FIG. 21, no binding to other TNF receptor family members was observed.

TABLE 29
Affinity by SPR

| Sample | Species | Ka(1/Ms) | Kd(1/s) | KD(M) |
|---|---|---|---|---|
| BGA-5623 | huCD137 | 2.12E+05 | 5.03E−03 | 2.38E−08 |
| | cynoCD137 | 1.30E+05 | 2.54E−03 | 1.96E−08 |

TABLE 30
Amino acid and DNA sequences

| | SEQ ID | | |
|---|---|---|---|
| Antibody | NO | | SEQUENCE |
| BGA-5623 | SEQ ID | VH | EVQLLESGGGLVQPGGSLRLSCAASGFTVSAEDVGWV |
| | NO: 84 | | RQAPGKGLEWVSAILDKGGSTYYAESVKGRFTISRDN |
| | | | AKNTLYLQMSSLRAEDTAVYYCARIVYHAGGGVTFDT |
| | | | RGQGTQVTVSS |
| | SEQ ID | VH | GAGGTCCAGTTACTTGAGAGTGGTGGAGGTCTGGTCC |
| | NO: 85 | DNA | AACCAGGAGGTTCGCTGCGTTTATCCTGCGCCGCGTC |
| | | | TGGATTCACGGTTTCCGCCGAAGACGTGGGTTGGGTG |
| | | | CGTCAAGCGCCGGGGAAAGGACTGGAATGGGTCTCCG |
| | | | CCATCTTGGATAAGGGTGGTTCGACATACTATGCGGA |
| | | | AAGTGTCAAAGGGCGCTTTACGATCTCGCGCGATAAC |
| | | | GCAAAAAATACTCTTTACCTTCAAATGTCTAGCCTTC |
| | | | GTGCTGAGGACACTGCGGTGTACTACTGCGCCCGCAT |
| | | | TGTCTACCATGCTGGTGGTGGCGTCACCTTTGATACT |
| | | | CGGGGACAGGGCACCCAAGTTACAGTCTCATCG |
| | SEQ ID | IgG1 | EVQLLESGGGLVQPGGSLRLSCAASGFTVSAEDVGWV |
| | NO: 283 | AA | RQAPGKGLEWVSAILDKGGSTYYAESVKGRFTISRDN |
| | | | AKNTLYLQMSSLRAEDTAVYYCARIVYHAGGGVTFDT |
| | | | RGQGTQVTVSSEPKSSDKTHTCPPCPAPELLGGPSVF |
| | | | LFPPKPKDTLMISRTPEVTCVVVDVSHEDPEVKFNWY |
| | | | VDGVEVHNAKTKPREEQYNSTYRVVSVLTVLHQDWLN |
| | | | GKEYKCKVSNKALPAPIEKTISKAKGQPREPQVYTLP |
| | | | PSRDELTKNQVSLTCLVKGFYPSDIAVEWESNGQPEN |
| | | | NYKTTPPVLDSDGSFFLYSKLTVDKSRWQQGNVFSCS |
| | | | VMHEALHNHYTQKSLSLSPGK |
| | SEQ ID | IgG1 | GAGGTCCAGTTACTTGAGAGTGGTGGAGGTCTGGTCC |
| | NO: 284 | DNA | AACCAGGAGGTTCGCTGCGTTTATCCTGCGCCGCGTC |
| | | | TGGATTCACGGTTTCCGCCGAAGACGTGGGTTGGGTG |
| | | | CGTCAAGCGCCGGGGAAAGGACTGGAATGGGTCTCCG |
| | | | CCATCTTGGATAAGGGTGGTTCGACATACTATGCGGA |
| | | | AAGTGTCAAAGGGCGCTTTACGATCTCGCGCGATAAC |
| | | | GCAAAAAATACTCTTTACCTTCAAATGTCTAGCCTTC |
| | | | GTGCTGAGGACACTGCGGTGTACTACTGCGCCCGCAT |
| | | | TGTCTACCATGCTGGTGGTGGCGTCACCTTTGATACT |
| | | | CGGGGACAGGGCACCCAAGTTACAGTCTCATCGGAGC |
| | | | CCAAATCTTCTGACAAAACTCACACATGCCCACCGTG |
| | | | CCCAGCACCTGAACTCCTGGGGGGACCGTCAGTCTTC |
| | | | CTCTTCCCCCCAAAACCCAAGGACACCCTCATGATCT |
| | | | CCCGGACCCCTGAGGTCACATGCGTGGTGGTGGACGT |
| | | | GAGCCACGAAGACCCTGAGGTCAAGTTCAACTGGTAC |
| | | | GTGGACGGCGTGGAGGTGCATAATGCCAAGACAAAGC |
| | | | CGCGGGAGGAGCAGTACAACAGCACGTACCGTGTGGT |
| | | | CAGCGTCCTCACCGTCCTGCACCAGGACTGGCTGAAT |
| | | | GGCAAGGAGTACAAGTGCAAGGTCTCCAACAAAGCCC |
| | | | TCCCAGCCCCCATCGAGAAAACCATCTCCAAAGCCAA |
| | | | AGGGCAGCCCCGAGAACCACAGGTGTACACCCTGCCC |
| | | | CCATCCCGGGATGAGCTGACCAAGAACCAGGTCAGCC |
| | | | TGACCTGCCTGGTCAAAGGCTTCTATCCCAGCGACAT |
| | | | CGCCGTGGAGTGGGAGAGCAATGGGCAGCCGGAGAAC |
| | | | AACTACAAGACCACGCCTCCCGTGCTGGACTCCGACG |
| | | | GCTCCTTCTTCCTCTACAGCAAGCTCACCGTGGACAA |
| | | | GAGCAGGTGGCAGCAGGGGAACGTCTTCTCATGCTCC |
| | | | GTGATGCATGAGGCTCTGCACAACCACTACACGCAGA |
| | | | AGAGCCTCTCCCTGTCTCCGGGTAAA |
| | SEQ ID | IgG4 | EVQLLESGGGLVQPGGSLRLSCAASGFTVSAEDVGWV |
| | NO: 285 | AA | RQAPGKGLEWVSAILDKGGSTYYAESVKGRFTISRDN |
| | | | AKNTLYLQMSSLRAEDTAVYYCARIVYHAGGGVTFDT |
| | | | RGQGTQVTVSSESKYGPPCPPCPAPEFLGGPSVFLFP |
| | | | PKPKDTLMISRTPEVTCVVVDVSQEDPEVQFNWYVDG |
| | | | VEVHNAKTKPREEQFNSTYRWSVLTVLHQDWLNGKEY |
| | | | KCKVSNKGLPSSIEKTISKAKGQPREPQVYTLPPSQE |
| | | | EMTKNQVSLTCLVKGFYPSDIAVEWESNGQPENNYKT |
| | | | TPPVLDSDGSFFLYSRLTVDKSRWQEGNVFSCSVMHE |
| | | | ALHNHYTQKSLSLSLGK |
| | SEQ ID | IgG4 | GAGGTCCAGTTACTTGAGAGTGGTGGAGGTCTGGTCC |
| | NO: 286 | DNA | AACCAGGAGGTTCGCTGCGTTTATCCTGCGCCGCGTC |
| | | | TGGATTCACGGTTTCCGCCGAAGACGTGGGTTGGGTG |
| | | | CGTCAAGCGCCGGGGAAAGGACTGGAATGGGTCTCCG |
| | | | CCATCTTGGATAAGGGTGGTTCGACATACTATGCGGA |
| | | | AAGTGTCAAAGGGCGCTTTACGATCTCGCGCGATAAC |
| | | | GCAAAAAATACTCTTTACCTTCAAATGTCTAGCCTTC |
| | | | GTGCTGAGGACACTGCGGTGTACTACTGCGCCCGCAT |
| | | | TGTCTACCATGCTGGTGGTGGCGTCACCTTTGATACT |
| | | | CGGGGACAGGGCACCCAAGTTACAGTCTCATCGGAAT |
| | | | CCAAGTATGGTCCGCCATGTCCACCGTGCCCGGCACC |
| | | | CGAGTTTTTGGGAGGGCCTAGTGTGTTTTTGTTTCCT |
| | | | CCTAAGCCAAAGGACACGCTTATGATTAGTCGCACAC |
| | | | CCGAGGTTACCTGTGTAGTGGTGGACGTATCACAAGA |
| | | | GGATCCGGAAGTCCAATTTAATTGGTACGTCGACGGA |
| | | | GTGGAAGTCCACAATGCAAAGACCAAGCCTAGGGAAG |
| | | | AACAGTTCAATTCTACCTATCGCTGGTCTGTACTCAC |
| | | | TGTACTCCACCAGGATTGGCTCAATGGAAAGGAATAC |
| | | | AAATGCAAGGTTTCCAACAAAGGGCTCCCCTCTTCCA |
| | | | TTGAGAAAACTATTAGCAAGGCTAAAGGCCAACCCAG |
| | | | AGAACCACAGGTTTATACCCTTCCACCTTCCCAGGAG |
| | | | GAGATGACGAAGAATCAGGTTAGTCTTACGTGCCTCG |
| | | | TTAAAGGATTCTACCCTTCAGATATCGCAGTTGAATG |
| | | | GGAGTCTAACGGGCAACCCGAGAACAACTACAAGACC |
| | | | ACCCCGCCGGTGCTGGACTCAGATGGTAGTTTCTTTC |
| | | | TTTACAGTCGGCTTACCGTCGACAAGTCACGCTGGCA |
| | | | GGAGGGAAATGTATTCTCCTGCAGCGTAATGCACGAA |
| | | | GCTCTGCACAATCACTATACCCAGAAGTCACTGAGCC |
| | | | TTTCTCTGGGTAAG |

### Example 23. Epitope Mapping of BGA-5623 by Alanine Scanning

To characterize the binding epitope of BGA-5623, 17 amino acid residues of human CD137 were mutated to alanine individually to generate 17 single-mutation huCD137 variants based upon the information from the crystal structure of CD137 reported previously (Bitra et al., (2018) J Biol Chem, 293, 9958-9969; Chin et al., (2018) Nat. Commun. 9, 4679).

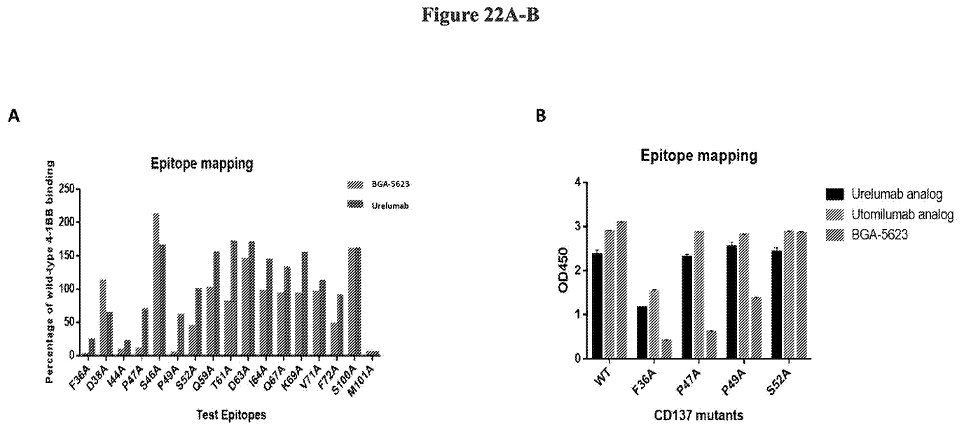

The CD137 mutants along with the wild-type CD137 were transiently expressed in HEK293 cells (ATCC CRL-1573). Their recognition and binding by BGA-5623 was analyzed by flow cytometry. An Urelumab analog (SEQ ID NOs: 287-290) that was generated in house by using the publicly available sequences of Urelumab, was used in the same assay to monitor the expression of CD137 mutants. In this assay, human CD137 or human CD137 mutant expressing cells (10⁵ cells/well) were incubated with 2 μg/ml of purified BGA-5623-mutFc (Fc fusion VH Ab) or Urelumab analog, followed by binding with Alexa Fluro-647-labeled anti-hu IgG Fc antibody (Cat.: 409320, BioLegend, USA). Cell fluorescence was quantified using a flow cytometer (Guava easyCyte™ 8HT, Merck-Millipore, USA). All results were normalized using the mean values of the fluorescence reading of wild type CD137 binding signal as the standard. To simplify data analysis, if an antibody's FACS binding signal for a specific mutant CD137 dropped to or below 25%, then the amino acid at that site was considered critical to the epitope. As shown in the FIG. 22A, the epitope of BGA-5623 has important residues for binding at amino acids F36, 144, P47, P49 and S52 of CD137.

In order to further explore the BGA-5623 epitope, human CD137 ECD mutants with single-AA substitution were expressed and purified to prepare for ELISA. In addition, a Utomilumab analog antibody (SEQ TD NOs: 291-294) was created in house by using the publicly available sequences of Utomilumab. The CD137 mutants along with the wild-type CD137 were analyzed for binding by BGA-5623 by direct ELISA. In brief, 50 ng each of wild-type or mutant CD137 was coated in an ELISA plate. After blocking, 100 μl of BGA-5623-mutFc, Urelumab analog or Utomilumab analog antibody at a concentration of 2 μg/ml was added to the plate and the binding signal of each antibody was detected by HRP-linked secondary antibody. In the ELISA binding assay using wild-type or mutant huCD137, amino acids F36A, P47A and P49A significantly impaired the binding of CD137 and BGA-5623 (FIGS. 22A-22B). Changes at amino acid F36A only slightly reduced the binding of the Urelumab or Utomilumab analogs, which indicates F36A plays a critical role in the conformation integrity of CD137. In contrast, neither changes at amino acids P47A or P49A disrupted the binding of the Urelumab or Utomilumab analog to CD137, indicating that BGA-5623, Urelumab analog or Utomilumab have different epitopes. This data indicated that amino acids F36A, P47A and P49A are critical residues in the epitope for antibody BGA-5623.

TABLE 31
Amino acid and DNA sequences

| | SEQ ID | | |
|---|---|---|---|
| Antibody | NO | | SEQUENCE |
| Urelumab | SEQ ID | Heavy | QVQLQQWGAGLLKPSETLSLTCAVYGGSFSGYYW |
| analog | NO: 287 | chain | SWIRQSPEKGLEWIGEINHGGYVTYNPSLESRVT |
| | | AA | ISVDTSKNQFSLKLSSVTAADTAVYYCARDYGPG |
| | | | NYDWYFDLWGRGTLVTVSSASTKGPSVFPLAPCS |
| | | | RSTSESTAALGCLVKDYFPEPVTVSWNSGALTSG |
| | | | VHTFPAVLQSSGLYSLSSVVTVPSSSLGTKTYTC |
| | | | NVDHKPSNTKVDKRVESKYGPPCPPCPAPEFLGG |
| | | | PSVFLFPPKPKDTLMISRTPEVTCVVVDVSQEDP |
| | | | EVQFNWYVDGVEVHNAKTKPREEQFNSTYRWSVL |
| | | | TVLHQDWLNGKEYKCKVSNKGLPSSIEKTISKAK |
| | | | GQPREPQVYTLPPSQEEMTKNQVSLTCLVKGFYP |
| | | | SDIAVEWESNGQPENNYKTTPPVLDSDGSFFLYS |
| | | | RLTVDKSRWQEGNVFSCSVMHEALHNHYTQKSLS |
| | | | LSLGK |
| | SEQ ID | Heavy | CAAGTTCAACTTCAGCAGTGGGGAGCGGGTCTCT |
| | NO: 288 | chain | TGAAGCCTAGTGAGACGCTGAGCCTGACCTGTGC |
| | | DNA | AGTCTACGGGGGTTCATTTTCTGGGTACTACTGG |
| | | | TCCTGGATACGCCAAAGCCCAGAGAAAGGCCTGG |
| | | | AATGGATTGGGGAGATAAATCACGGGGGTTATGT |
| | | | GACATATAATCCTTCTCTTGAGAGCCGGGTCACG |
| | | | ATATCTGTTGACACAAGTAAAAATCAGTTCTCCC |
| | | | TCAAACTGAGCTCCGTTACCGCAGCAGACACTGC |
| | | | CGTTTACTACTGTGCGCGGGACTATGGGCCAGGT |
| | | | AATTACGATTGGTACTTCGACCTGTGGGGTAGAG |
| | | | GAACGCTGGTCACTGTAAGCAGTGCATCTACAAA |
| | | | GGGCCCCAGCGTGTTTCCCCTTGCACCATGTTCA |
| | | | CGGAGCACTAGTGAAAGCACTGCCGCGCTCGGGT |
| | | | GTCTCGTTAAGGATTATTTTCCGGAGCCTGTCAC |
| | | | TGTCTCTTGGAACTCTGGTGCACTGACATCAGGC |
| | | | GTTCACACGTTCCCTGCAGTACTCCAGAGCTCAG |
| | | | GATTGTACAGCCTGTCCAGTGTAGTTACAGTGCC |
| | | | TTCCTCCTCCCTTGGAACAAAGACCTACACCTGC |
| | | | AACGTGGACCATAAGCCCAGCAATACGAAAGTAG |
| | | | ACAAACGAGTCGAATCCAAGTATGGTCCGCCATG |
| | | | TCCACCGTGCCCGGCACCCGAGTTTTTGGGAGGG |
| | | | CCTAGTGTGTTTTTGTTTCCTCCTAAGCCAAAGG |
| | | | ACACGCTTATGATTAGTCGCACACCCGAGGTTAC |
| | | | CTGTGTAGTGGTGGACGTATCACAAGAGGATCCG |
| | | | GAAGTCCAATTTAATTGGTACGTCGACGGAGTGG |
| | | | AAGTCCACAATGCAAAGACCAAGCCTAGGGAAGA |
| | | | ACAGTTCAATTCTACCTATCGCTGGTCTGTACTC |
| | | | ACTGTACTCCACCAGGATTGGCTCAATGGAAAGG |
| | | | AATACAAATGCAAGGTTTCCAACAAAGGGCTCCC |
| | | | CTCTTCCATTGAGAAAACTATTAGCAAGGCTAAA |
| | | | GGCCAACCCAGAGAACCACAGGTTTATACCCTTC |
| | | | CACCTTCCCAGGAGGAGATGACGAAGAATCAGGT |
| | | | TAGTCTTACGTGCCTCGTTAAAGGATTCTACCCT |
| | | | TCAGATATCGCAGTTGAATGGGAGTCTAACGGGC |
| | | | AACCCGAGAACAACTACAAGACCACCCCGCCGGT |
| | | | GCTGGACTCAGATGGTAGTTTCTTTCTTTACAGT |
| | | | CGGCTTACCGTCGACAAGTCACGCTGGCAGGAGG |
| | | | GAAATGTATTCTCCTGCAGCGTAATGCACGAAGC |
| | | | TCTGCACAATCACTATACCCAGAAGTCACTGAGC |
| | | | CTTTCTCTGGGTAAG |
| | SEQ ID | Light | EIVLTQSPATLSLSPGERATLSCRASQSVSSYLA |
| | NO: 289 | chain | WYQQKPGQAPRLLIYDASNRATGIPARFSGSGSG |
| | | AA | TDFTLTISSLEPEDFAVYYCQQRSNWPPALTFGG |
| | | | GTKVEIKRTVAAPSVFIFPPSDEQLKSGTASVVC |
| | | | LLNNFYPREAKVQWKVDNALQSGNSQESVTEQDS |
| | | | KDSTYSLSSTLTLSKADYEKHKVYACEVTHQGLS |
| | | | SPVTKSFNRGEC |
| | SEQ ID | Light | GAAATCGTGCTGACCCAATCACCTGCGACCCTCT |
| | NO: 290 | chain | CCCTTAGCCCTGGAGAACGAGCAACTCTCAGTTG |
| | | DNA | CAGAGCAAGCCAGAGCGTCAGCTCATATCTCGCA |
| | | | TGGTATCAACAAAAACCAGGCCAAGCTCCTAGAC |
| | | | TGTTGATTTATGATGCCTCTAACCGGGCGACTGG |
| | | | CATACCAGCCCGCTTTAGCGGGTCTGGGAGTGGC |
| | | | ACTGATTTTACGCTCACGATTTCATCTTTGGAGC |
| | | | CAGAAGATTTCGCTGTATACTACTGTCAACAAAG |
| | | | GTCAAACTGGCCGCCTGCACTTACGTTCGGCGGG |
| | | | GGTACCAAGGTGGAGATTAAGAGGACAGTTGCGG |
| | | | CGCCATCAGTTTTTATATTCCCACCCTCCGATGA |
| | | | GCAGTTGAAAAGTGGCACGGCATCAGTTGTCTGT |
| | | | CTGCTGAACAATTTCTATCCACGGGAGGCGAAAG |
| | | | TCCAATGGAAAGTGGACAATGCGCTTCAGAGCGG |
| | | | GAACTCTCAGGAGAGTGTCACTGAACAGGATTCA |
| | | | AAGGACTCCACGTACTCACTCTCATCCACTCTCA |
| | | | CCCTTTCAAAGGCGGATTACGAAAAGCATAAGGT |
| | | | CTACGCTTGTGAGGTAACACATCAGGGGCTTTCC |
| | | | AGTCCTGTAACCAAGAGCTTCAATCGCGGCGAAT |
| | | | GC |
| Utomilumab | SEQ ID | Heavy | EVQLVQSGAEVKKPGESLRISCKGSGYSFSTYWI |
| analog | NO: 291 | chain | SWVRQMPGKGLEWMGKIYPGDSYTNYSPSFQGQV |
| | | AA | TISADKSISTAYLQWSSLKASDTAMYYCARGYGI |
| | | | FDYWGQGTLVTVSSASTKGPSVFPLAPCSRSTSE |
| | | | STAALGCLVKDYFPEPVTVSWNSGALTSGVHTFP |
| | | | AVLQSSGLYSLSSVVTVPSSNFGTQTYTCNVDHK |
| | | | PSNTKVDKTVERKCCVECPPCPAPPVAGPSVFLF |
| | | | PPKPKDTLMISRTPEVTCVVVDVSHEDPEVQFNW |
| | | | YVDGVEVHNAKTKPREEQFNSTFRVVSVLTVVHQ |
| | | | DWLNGKEYKCKVSNKGLPAPIEKTISKTKGQPRE |
| | | | PQVYTLPPSREEMTKNQVSLTCLVKGFYPSDIAV |
| | | | EWESNGQPENNYKTTPPMLDSDGSFFLYSKLTVD |
| | | | KSRWQQGNVFSCSVMHEALHNHYTQKSLSLSPGK |
| | SEQ ID | Heavy | GAAGTTCAACTGGTACAGTCCGGTGCTGAAGTTA |
| | NO: 292 | chain | AAAAACCCGGAGAGAGTCTGAGAATCTCTTGCAA |
| | | DNA | GGGGTCTGGGTACTCTTTTAGTACATATTGGATA |
| | | | TCTTGGGTGAGACAAATGCCGGGGAAAGGCCTCG |
| | | | AATGGATGGGCAAGATTTATCCCGGAGACAGCTA |
| | | | TACAAATTACTCACCTAGTTTTCAAGGTCAGGTA |
| | | | ACTATCAGCGCGGACAAATCCATAAGTACGGCCT |
| | | | ATCTGCAATGGTCTTCCTTGAAGGCATCCGACAC |
| | | | GGCGATGTATTACTGCGCAAGAGGGTATGGTATA |
| | | | TTCGATTACTGGGGGCAGGGCACTCTCGTTACCG |
| | | | TGAGCAGCGCTTCAACAAAAGGCCCATCTGTCTT |
| | | | CCCTCTCGCGCCTTGCTCCCGCTCTACTTCAGAG |
| | | | TCAACAGCTGCTCTGGGGTGCTTGGTGAAGGATT |
| | | | ACTTTCCTGAACCGGTTACTGTCAGCTGGAATTC |
| | | | TGGGGCTTTGACGAGCGGAGTTCACACTTTCCCA |
| | | | GCTGTACTGCAAAGTTCTGGACTTTACAGCTTGA |
| | | | GCTCCGTGGTGACAGTGCCGAGCTCAAACTTCGG |
| | | | TACGCAGACATATACATGCAATGTGGATCATAAA |
| | | | CCCAGCAATACGAAAGTAGATAAGACAGTCGAAA |
| | | | GGAAGTGTTGTGTAGAGTGCCCACCGTGCCCCGC |
| | | | CCCTCCGGTTGCAGGCCCATCAGTGTTTCTCTTC |
| | | | CCGCCCAAACCTAAGGACACGCTGATGATTTCAC |
| | | | GAACGCCCGAGGTCACCTGTGTCGTAGTCGATGT |
| | | | GTCCCATGAGGACCCAGAAGTTCAGTTCAATTGG |
| | | | TATGTAGACGGCGTGGAGGTACACAATGCAAAAA |
| | | | CGAAACCACGCGAGGAACAGTTCAATAGTACGTT |
| | | | CCGGGTCGTGAGTGTGCTCACGGTGGTTCACCAA |
| | | | GACTGGCTGAACGGGAAAGAATATAAGTGCAAAG |
| | | | TATCCAATAAAGGCCTGCCCGCACCCATCGAAAA |
| | | | AACTATAAGTAAAACTAAAGGGCAACCGCGAGAG |
| | | | CCTCAGGTCTATACTTTGCCACCGTCACGCGAAG |
| | | | AGATGACCAAGAACCAAGTAAGTCTTACTTGCTT |
| | | | GGTCAAGGGGTTCTACCCCTCAGATATAGCGGTG |
| | | | GAGTGGGAATCCAATGGCCAACCCGAGAACAATT |
| | | | ACAAGACAACACCACCAATGCTGGACTCTGACGG |
| | | | GAGTTTCTTTCTGTATAGTAAACTTACCGTCGAT |
| | | | AAATCACGCTGGCAGCAAGGCAACGTATTCTCAT |
| | | | GCTCAGTAATGCATGAAGCCTTGCATAATCACTA |
| | | | CACTCAGAAAAGCCTGTCCCTCTCTCCAGGTAAG |
| | SEQ ID | Light | SYELTQPPSVSVSPGQTASITCSGDNIGDQYAHW |
| | NO: 293 | chain | YQQKPGQSPVLVIYQDKNRPSGIPERFSGSNSGN |
| | | AA | TATLTISGTQAMDEADYYCATYTGFGSLAVFGGG |
| | | | TKLTVLGQPKAAPSVTLFPPSSEELQANKATLVC |
| | | | LISDFYPGAVTVAWKADSSPVKAGVETTTPSKQS |
| | | | NNKYAASSYLSLTPEQWKSHRSYSCQVTHEGSTV |
| | | | EKTVAPTECS |
| | SEQ ID | Light | AGCTACGAGTTGACTCAGCCACCATCCGTCAGTG |
| | NO: 294 | chain | TTAGCCCCGGACAGACGGCGTCAATTACTTGTAG |
| | | DNA | TGGAGACAACATTGGCGATCAGTACGCCCATTGG |
| | | | TATCAGCAAAAACCGGGTCAATCACCAGTTCTGG |
| | | | TGATATATCAGGATAAGAATCGACCATCAGGGAT |
| | | | TCCAGAGAGGTTCTCAGGTAGTAACTCAGGTAAC |
| | | | ACAGCTACTCTGACCATAAGTGGTACCCAGGCTA |
| | | | TGGACGAAGCAGACTACTACTGCGCCACCTACAC |
| | | | AGGCTTCGGATCACTTGCGGTTTTCGGGGGCGGA |
| | | | ACCAAACTAACCGTTCTTGGGCAACCTAAGGCTG |
| | | | CCCCGTCCGTGACTCTTTTTCCTCCGTCTTCCGA |
| | | | AGAGCTTCAAGCCAATAAAGCAACCTTGGTATGT |
| | | | TTGATTTCCGATTTTTACCCTGGAGCGGTGACGG |
| | | | TGGCATGGAAAGCTGATTCAAGCCCTGTGAAAGC |
| | | | TGGGGTGGAAACTACTACGCCGTCCAAACAGAGC |
| | | | AATAATAAGTACGCGGCCTCCAGCTATTTGTCAC |
| | | | TTACCCCGGAGCAATGGAAGTCACACCGAAGTTA |
| | | | CAGTTGTCAAGTTACTCACGAAGGCAGCACTGTT |
| | | | GAAAAGACCGTAGCACCCACCGAATGCTCA |

### Example 24. Ligand Competition

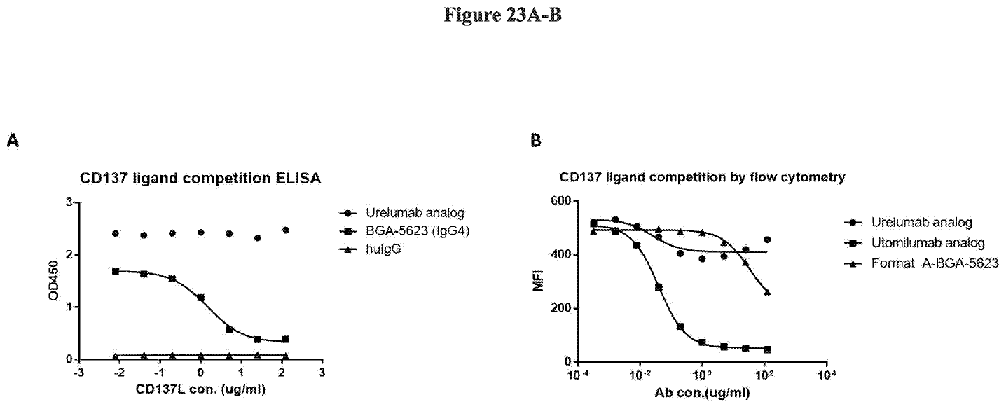

Human CD137 binds to its major ligand human CD137 ligand (CD137L) with weak affinity at an approximate Kd of three-digit M (Chin et al., (2018) Nat Commun 9, 4679). The epitope mapping results in Example 23 above, shows that amino acid residues F36A, P47A and P49A of CD137 are critical amino acid residues that make up part of the epitope for the BGA-5623 antibody. In addition, the ligand binds CD137 along the entire length of receptor CRD-2 and the A2 motif of CRD-3, and the interface between the receptor and ligand is primarily mediated by hydrogen bonds and van der Waals interactions (Bitra et al., (2018) J Biol Chem, 293, 9958-9969). Based on this data, it was hypothesized that the BGA-5623 antibody can block CD137/CD137 ligand interaction. BGA-5623 was generated with a human IgG4 Fc fusion. For CD137 ligand competition ELISA, a Maxisorp immunoplate was coated with human CD137 ECD-mIgG2a and blocked with 300 BSA (w/v) in PBS buffer (blocking buffer). VH domain antibody BGA-5623 was blocked with blocking buffer for 30 minutes and added to wells of the ELISA plate for 1 hour in the presence of serially diluted human CD137 ligand ECD-mIgG2a. After washes with PBST, bound antibodies were detected using HRP-conjugated anti-human IgG antibody (Sigma, A0170) and 3,3′,5,5′-tetramethylbenzidine substrate (Cat.: 00-4201-56, eBioscience, USA) (FIG. 23A). For the assay of CD137 ligand competition by flow cytometry, a CD137 stably transduced cell line Hut78/huCD137 was incubated with human CD137 ligand ECD-mIgG2a in the presence of serially diluted BGA-5623, followed by detection with goat-anti-murine IgG-APC (FIG. 23B). As shown in FIG. 23A-B, BGA-5623 competes with CD137 ligand and reduces CD137/CD137 ligand interaction.

### Example 25. Structural and Functional CD137 Epitope Mapping

To better understand how the anti-CD137 single domain antibody arm is capable of high affinity binding for CD137, and robust agonist of CD137/CD137L interaction, the crystal structure of VH (BGA-5623) in complex with CD137 was determined.

### A. CD137 and VH (BGA-5623) Expression, Purification, and Crystallization

Human CD137 ectodomain containing four CRDs (1-4; amino acids 24-162) harboring C121S, N138D, and N149Q mutations was expressed in HEK293G cells. The cDNA coding CD137 was cloned into in house expression vector with an N-terminal secretion sequence and a C-terminal TEV cleavage site followed by an Fc tag. The culture supernatant containing the secreted CD137-Fc fusion protein was mixed with Mab Select Sure™ resin (GE Healthcare Life Sciences) for 3 hours at 4° C. The protein was washed with buffer containing 20 mM Tris-HCl pH 8.0, 150 mM NaCl, then eluted with 50 mM acetic acid (adjust pH value to 3.5 with 5 M NaOH), and finally neutralized with 1/10 CV 1.0M Tris-HCl pH8.0. The eluted protein was mixed with TEV proteases (10:1 molar ratio) and dialyzed against buffer (20 mM Tris-HCl, pH 8.0, 100 mM NaCl) at 4° C. overnight. The mixture was loaded onto a Ni-NTA column (Qiagen) and Mab Select Sure™ resin to remove the TEV proteases and Fc tag, and then the flow-through was further purified by size-exclusion chromatography in buffer (20 mM Tris pH 8.0, 100 mM NaCl) using a HiLoad 16/600 Superdex™ 75 μg column (GE Healthcare Life Sciences).

DNA sequence encoding VH (BGA-5623) was cloned into a PET21a vector with N-terminal HIS-MBP tag followed by TEV protease site. Protein expression in Shuffle T7 was induced at OD600 of 0.6-1.0 with 1 mM IPTG at 18° C. for 16 h. The cells were harvested by centrifugation at 7,000 g, 10 min. The cell pellets were re-suspended in lysis buffer (50 mM Na₃PO₄ pH 7.0, 300 mM NaCl) and lysed under sonication on ice. The lysate then was centrifuged at 48,000 g at 4° C. for 30 min. The supernatant was mixed with Talon resin and batched at 4° C. for 3 hours. The resin was washed with lysis buffer containing 5 mM imidazole, the protein was eluted in lysis buffer with additional 100 mM imidazole. The eluate was mixed with TEV proteases (10:1 molar ratio) and dialyzed against buffer (20 mM Tris-HCl, pH 8.0, 100 mM NaCl) at 4° C. overnight. The mixture was loaded onto a Talon column to remove the TEV proteases and HIS-MBP tag, and then the flow-through was further purified by size-exclusion chromatography in buffer (20 mM Tris pH 8.0, 100 mM NaCl) using a HiLoad 16/600 Superdex™ 75 μg column (GE Healthcare Life Sciences).

Purified CD137 was mixed with an excess of purified VH (BGA-5623) (1:1.5 molar ratio) to generate the CD137/VH (BGA-5623) complex. The complex was then further purified by gel filtration in buffer (20 mM Tris pH 8.0, 100 mM NaCl) using a HiLoad 16/600 Superdex™ 75 μg column (GE Healthcare Life Sciences). The CD137/VH (BGA-5623) complex (10 mg/ml) was crystallized in 0.6 M Li₂SO₄, 0.01 M NiCl₂, 0.1 M Tris pH 9.0. Crystals cryoprotected with stepwise 5% D-(+)-Sucrose to a final 20% concentration were flash frozen in liquid nitrogen. Besides, the apoVH (BGA-5623) was crystallized in 1.2 M (NH₄)₂SO₄, 0.1 M Citric Acid pH 5.0. Crystal was cryoprotected with 7% glycerol and flash frozen in liquid nitrogen. The X-ray diffraction data was collected at beamline BL45XU at Spring-8 synchrotron radiation facility (Hyogo, Japan).

### B. Data Collection and Structure Solution

The X-ray diffraction data was collected under cryo cooled conditions at 100 Kelvin at beamline BL45XU equipped with ZOO (Hirata, K., et al., Acta Crystallogr D Struct Biol, 2019. 75(Pt 2): 138-150) automated data collection system in Spring-8 synchrotron radiation facility (Hyogo, Japan). Diffraction images were processed with the integrated data processing software KAMO (Yamashita, et al., Acta Crystallogr D Struct Biol, 2018. 74 (Pt 5): 441-449) employing XDS (Kabsch W., Acta Crystallogr D Biol Crystallogr, 2010. 66 (Pt 2): 125-32). The structure of human CD137 (PDB: 6MGP) and VHH model (PDB:4U3X) were used as search models. The initial solution was found with molecular replacement program PHASER (McCoy et al., Phaser crystallographic software. J Appl Crystallogr, 2007. 40(Pt 4): 658-674). Then this model was iterative manually built with program COOT (Emsley et al., Acta Crystallogr D Biol Crystallogr, 2004. 60(Pt 12 Pt 1): 2126-32) and refinement using PHENIX (Adams et al., Acta Crystallogr D Biol Crystallogr, 2010. 66(Pt 2): 213-21). The final model was refined to acceptable R and R free values and Ramachandran statistics (calculated by Molprobity). Data processing and refinement statistics can be found in Table 32.

### C. The Structure of VH (BGA-5623) Bound to Human CD137

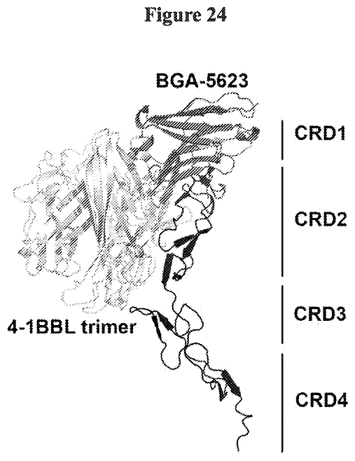

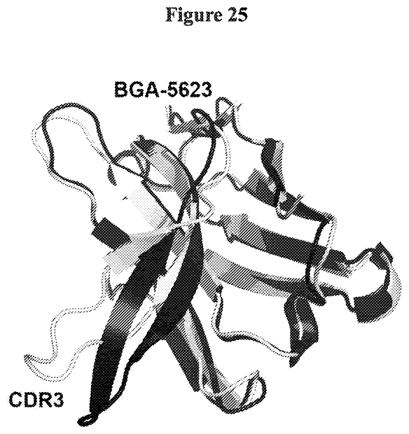

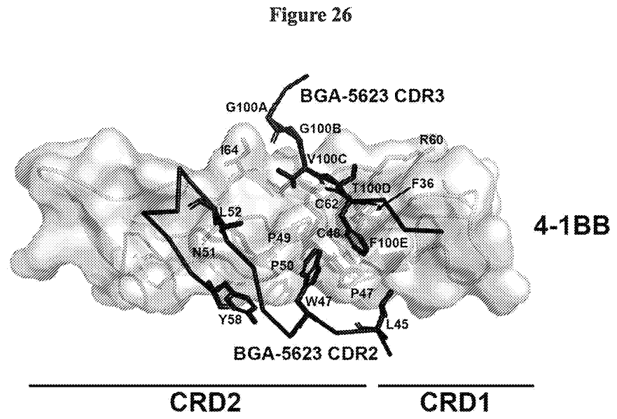

The VH (BGA-5623) in complex with CD137 crystallized in the 141 space group, with one complex in the asymmetric unit, and diffracted to 2.58 Å. The structure of VH (BGA-5623) bound to human CD137 shows that VH (BGA-5623) partially sterically interfaces with CD137L binding (FIG. 24). The buried surface area between VH (BGA-5623) and CD137 is approximately 571 A2. VH (BGA-5623) interactions are clustered around CD137 CRD2 domain. These interactions are primarily mediated by VH (BGA-5623) CDR2 and CDR3 and make more extensive contact with CD137. VH (BGA-5623) CDR1 does not directly contact CD137 while CDR3 undergoes a dramatic conformational change from unstructured loop to β-sheet upon CD137 binding (FIG. 25). VH (BGA-5623) CDR2 Leu52, Tyr58 contact CD137 residues Pro50, Asn51. VH (BGA-5623) CDR3 residues Gly100A, Gly100B, Val100C, Thr100D, Phe100E contact CD137 residues Phe36, Pro47, Pro49, Arg60, Cys62, Ile64. Besides, FR2 Leu45 and Trp47 contact CD137 residues Pro47, Cys48, Pro49, Pro50 which contribute significantly to CD137 binding. VH (BGA-5623) interacts with CD137 using a combination of hydrogen bonds and hydrophobic interactions. For example, FR2 Trp47 forms strong hydrophobic contacts with CD137 residues Pro47, Cys48, Pro49 and Pro50. CDR3 residue Phe100E forms hydrophobic interactions with CD137 residues Phe36 and Pro47. FR2 residue Trp47 and CDR3 residue Gly100A form one hydrogen bond with CD137 residues Pro47 and Ile64, respectively. CDR3 residue Val100C forms two hydrogen bonds with CD137 residue Cys62 (FIG. 26).

Based on the crystal structure of the VH (BGA-5623)/CD137 complex, the residues of CD137 that are contacted by VH (BGA-5623) (i.e., the epitopic residues of CD137 bound by VH) and the residues of VH (BGA-5623) that are contacted by CD137 (i.e. the paratopic residues of VH contacted by CD137) were determined. Table 33, below, show the residues of CD137 and VH (BGA-5623) to which they contact, as assessed using a contact distance stringency of 3.7 Å, a point at which van der Waals (non-polar) interaction forces are highest. This crystal structure analysis agreed with the previous alanine scanning analysis, with many of the CD137 residues that most affected BGA-5623 binding found to interact with BGA-5623 in the structure.

TABLE 32
Data collection and refinement statistics

| | apoVH | VH (BGA-5623)/ |
|---|---|---|
| Crystals | (BGA-5623) | CD137 complex |
| Data collection | | |
| X-ray source | BL45XU | BL45XU |
| Wavelength (Å) | 1.0 | 1.0 |
| Space group | P41212 | I41 |
| Cell Dimensions | | |
| a (Å) | 68.342 | 123.943 |
| b (Å) | 68.342 | 123.943 |
| c (Å) | 102.952 | 57.877 |
| α (°) | 90 | 90 |
| β (°) | 90 | 90 |
| γ (°) | 90 | 90 |
| Resolution (Å) | 56.94-1.70 (1.80-1.70)a | 87.64-2.58 (2.73-2.58) |
| Rsym (%) | 18.0 (145.8) | 21.4 (209.3) |
| <I/σ> | 14.3 (1.6) | 12.3 (1.2) |
| CC½ | 99.8 (56.1) | 99.7 (44.4) |
| Redundancy | 11.6 (5.9) | 6.8 (6.6) |
| Completeness (%) | 97.3 (84.3) | 99.9 (99.4) |
| Refinement | | |
| Resolution (Å) | 48.33-1.70 | 43.82-2.58 |
| No. reflections | 26791 | 14038 |
| Rwork/Rfree b(%) | 19.84/23.46 | 22.8/28.9 |
| r.m.s.d. cbonds/ | 0.006/0.892 | 0.010/1.314 |
| angles | | |
| Number of atoms | | |
| Protein | 1787 | 1959 |
| Water | 204 | 5 |
| B factors | | |
| Protein | 27.37 | 83.97 |
| Water | 36.42 | 66.79 |
| Ramachandran | | |
| Favored (%) | 98.71 | 94.21 |
| Allowed (%) | 1.29 | 3.47 |
| Outlier (%) | 0 | 2.32 |

aValues in parentheses are those of the highest resolution shell.
bCalculated from about 5% of the reflection set aside during refinement
cr.m.s.d., root mean square deviation

TABLE 33
Epitopic residues of CD137 and their corresponding
paratopic residues of VH(BGA-5623)

| CD137 | | VH (BGA-5623) | |
|---|---|---|---|
| Phe | 36 | Thr | 100D |
| | | Phe | 100E |
| Pro | 47 | Leu | 45 |
| | | Trp | 47 |
| | | Phe | 100E |
| Cys | 48 | Trp | 47 |
| Pro | 49 | Trp | 47 |
| | | Val | 100C |
| | | Thr | 100D |
| Pro | 50 | Trp | 47 |
| | | Tyr | 58 |
| Asn | 51 | Leu | 52 |
| Arg | 60 | Thr | 100D |
| Cys | 62 | Gly | 100B |
| | | Val | 100C |
| Ile | 64 | Gly | 100A |

The VH (BGA-5623) residues are numbered in Kabat nomenclature.

### Example 27. Parameters which May Influence In Vitro CD137 Activation

It has been demonstrated that, in addition to affinities, receptor density and epitope location in CD137 and molecular format, there are other key parameters such as module ratios, module orientation, linker length and Fc functions that could significantly affect cytokine release (IL-2 and IFN-7). Therefore, to inform rational design of CD137 based multispecific antibodies, we took a systematic approach to interrogate how these parameters influence CD137 agonism. Expression and preparation of these multispecific antibodies were carried out as described.

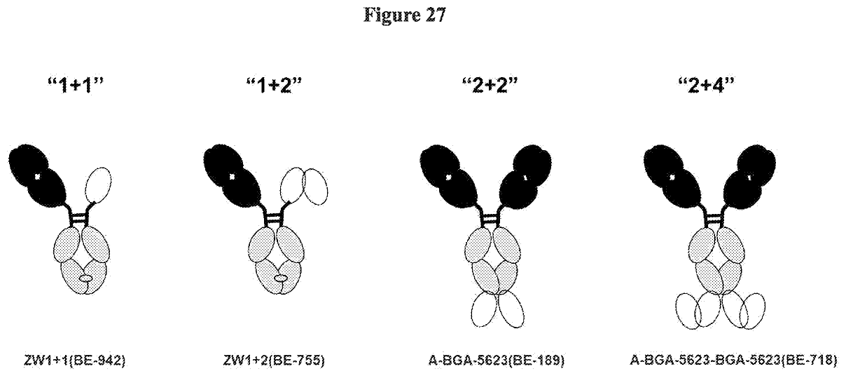

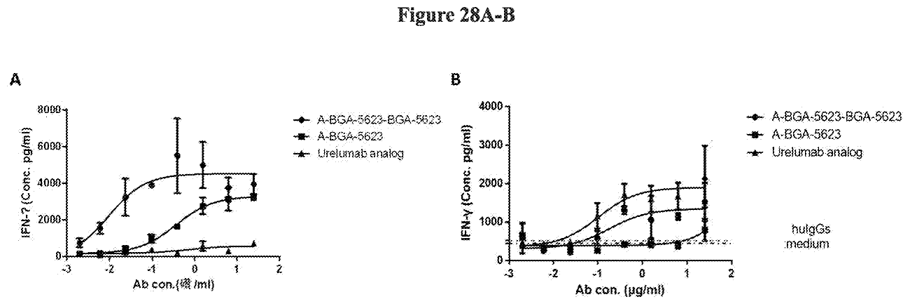

First, we constructed CEA/CD137 multispecific antibody variants with different module ratios such as 2:4, 1:1 and 1:2, namely BE-718 (A-BGA-5623-BGA-5623) (SEQ ID NOs: 295 and 179), BE-942 (ZW 1+1) (SEQ ID NOs: 299, 301 and 303), which is BGA-5623 in the 1+1 configuration and BE-755 (ZW1+2) (SEQ ID NOs: 299, 301 and 305) which is BGA-5623 in the 1+2 configuration (FIG. 27). For antibody constructs with a “ZW” designation, an inert Fc was used for these multispecific antibodies and the Azymetric™ Platform from Zymeworks was utilized to assemble the Fab×VH configuration, in which ZW1 mutations (chain A: T350V/L351Y/F405A/Y407V; chain B: T350V/T366L/K392L/T394W) were introduced in the CH3 domain of heavy chain to allow efficient heterodimer formation (Von Kreudenstein et al., (2013) Mabs 5(5):646-54). For BE-189 (A-BGA-5623) (SEQ ID NOs: 255 and 179), which represents the multispecific antibody with a module ratio of 2:2, we were able to investigate how the module ratio influences cytokine release. As described above in Example 20, the high CEA expressing cell line, CT26/CEA, together with PBMCs (2×10⁵/well) and HEK293/OS8 cells, which could trigger the first signal for T-cell activation were used for an in vitro CD137 activation assay. As shown in Table 34 and FIG. 28A-B, the multispecific antibody of a module ratio of 2:2 was demonstrated to be a potent CD137 agonist without CD137 intrinsic activation, which suggests BE-189 (Format A-BGA-5623) activates CD137 in a CEA dependent way. In contrast, the multispecific antibody BE-718 (A-BGA-5623-BGA-5623) with a module ratio of 2:4, was shown to activate CD137 even in the absence of CEA expressing cells.

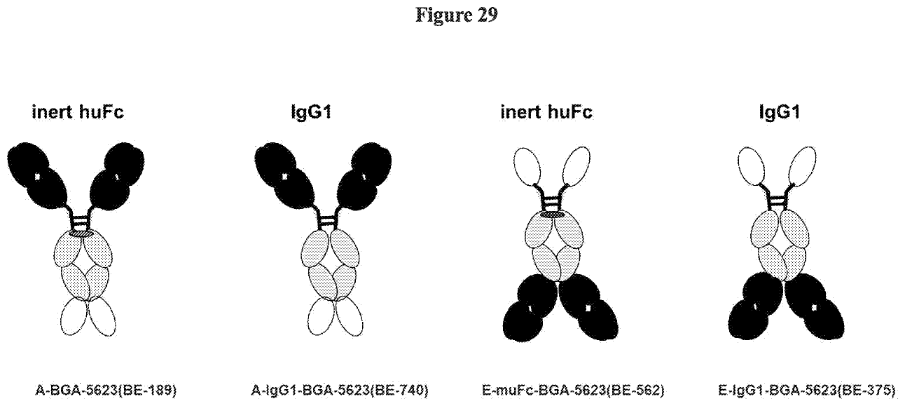

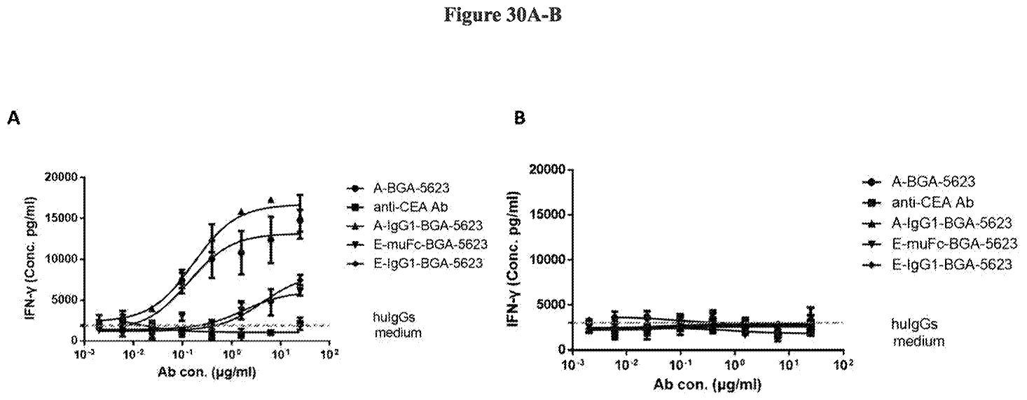

Next, we then investigated how module orientation and Fc functions influence CD137 activation. In this experiment, we constructed BE-740 (A-IgG1-BGA-5623) (SEQ ID NOs: 297 and 179), which was exactly the same as A-BGA-5623 (BE-189) in the format except for a wild-type IgG1 Fc was used to substitute the inert Fc. We also constructed BE-562 (E-muFc-BGA-5623) (SEQ ID NOs: 307 and 179) and BE-375 (E-IgG1-BGA-5623) (SEQ ID NOs: 309 and 179), respectively. As shown in FIG. 29, these two multispecific antibodies share the same pair of anti-CEA antibodies and anti-huCD137 VH domain (CEA and BGA-5623) as A-BGA-5623 and A-IgG1-BGA-5623, but with the opposite orientation. As described in Example 20, a PBMC based cytokine release assay was used to quantify the potency of CD137 activation. Based on the in vitro results, A-BGA-5623 and A-IgG1-BGA-5623 were demonstrated to be more potent in CD137 activation than E-muFc-BGA-5623 and E-IgG1-BGA-5623. In addition, based on this experiment, the Fc function seems to have minimal influence on CD137 activation (FIG. 30).

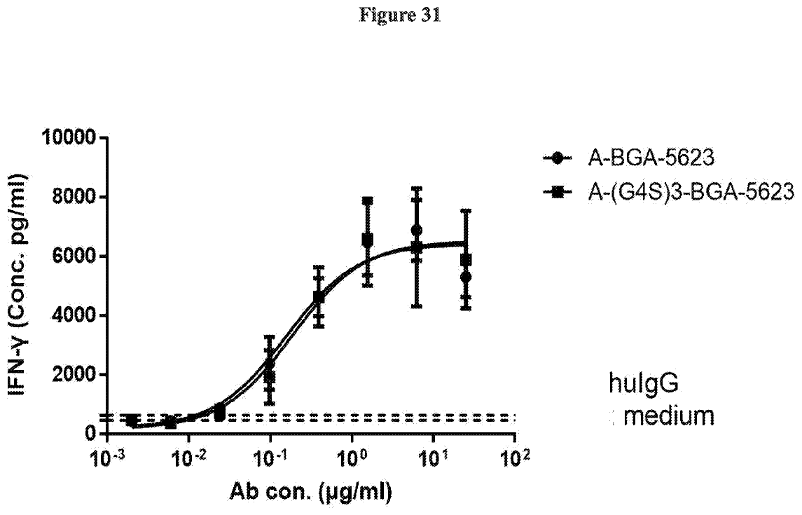

Then, the linker connecting Fc and VH domain antibody was evaluated for its influence on CD137 activation. A-(G4S)3-BGA-5623 (BE-244) (SEQ ID NOs: 311 and 179) was created with substitution of G4S with a 15 AA linker of (G4S)3 (SEQ ID NO: 329). Again, the PBMC based cytokine release assay was used to compare the potency. As shown in FIG. 31 and Table 34, the linker length has minimal influence in CD137 activation.

Lastly, two BGA-4712 variants (BGA-6468 and BGA-9442) with various affinities were selected for the potency comparison. SPR study and FACS analysis were shown in Table 35. For flow cytometry, human CD137⁺ or human CEA⁺ expressing cells (10⁵ cells/well) were incubated with various concentrations of purified VH domain antibodies, followed by binding with Alexa Fluro-647-labeled anti-hu IgG Fc antibody (Cat.: 409320, BioLegend, USA). Cell fluorescence was quantified using a flow cytometer (Guava easyCyte™ 8HT, Merck-Millipore, USA). There was no significant difference in CEA binding among two tested multispecific antibodies, whereas different binding affinities towards human CD137 were observed by flow cytometry as expected. Next, a PBMC based cytokine release assay as described above in Example 20 was applied to evaluate the potency of these BGA-4712 variants with different affinities in the multispecific antibody Format A-CD137/CEA. As shown in Table 35, the CD137 activation induced by these variants is proportionate to the increasing affinities of the CD137 arm.

TABLE 34
Key parameters for in vitro CD137 activation

| | | | | | SEC-HPLC | Functional |
|---|---|---|---|---|---|---|
| | | | | | (%) after | assay |
| | Alternative | Pair of | Module | | Protein A | EC50 |
| Code | name | mAbs | ratio | Fc | purification | (ug/ ml) |
| BE-189 | A-BGA-5623 | BGA-5623/CEA | 2 + 2 | Inert Fc | 94.55 | 0.31 |
| BE-718 | A-BGA-5623- | | 2 + 4 | Inert Fc | 89.47 | 0.01 |
| | BGA-5623 | | | | | |
| BE-740 | A-IgG1- BGA- | | 2 + 2 | wt-IgG1 | 98.71 | 0.17 |
| | 5623 | | | | | |
| BE-942 | ZW1 + 1 | | 1 + 1 | LALADS | 92.65 | 25.06 |
| BE-755 | ZW1 + 2 | | 1 + 2 | LALADS | 83.19 | 1.16 |
| BE-562 | E-muFc- BGA- | | 2 + 2 | Inert Fc | 98.17 | 4.69 |
| | 5623 | | | | | |
| BE-375 | E-IgG1- BGA- | | 2 + 2 | wt-IgG1 | 97.76 | 1.76 |
| | 5623 | | | | | |
| BE-244 | A-(G4S)3- BGA- | | 2 + 2 | Inert Fc | 98.84 | 0.18 |
| | 5623 | | | | | |

TABLE 35
Comparison of BGA-4712 variants in the multispecific
antibody format A-CD137xCEA with different affinities

| | | Cell binding | Functions | |
|---|---|---|---|---|
| | | EC50 (ug/ml) | EC50 | |
| Format/clone | Format | Hut78/huCD137 | (ug/ml) | Top |
| Format | A-CD137xCEA | 0.38 | 0.3412 | 2512 |
| A-BGA-5623 | | | | |
| Format | | 0.21 | 0.07681 | 1953 |
| A BGA-3386 | | | | |

TABLE 36
Amino acid and DNA sequences of CD137xCEA
multispecific antibodies

| CONSTRUCT | SEQ ID NO: | SEQUENCE |
|---|---|---|
| BE-718 | SEQ ID NO: 295 | QVQLVQSGAEVKKPGASVKVSCKASGYIFTSYYLHW |
| A-BGA-5623- | | VRQAPGQGLEWIGYINPQTGKTSYAQKFQGRVTMTR |
| BGA-5623 | | DTSTSTVYMELSSLRSEDTAVYYCAREYGNYNYPLD |
| heavy chain | | YWGQGTLVTVSSASTKGPSVFPLAPSSKSTSGGTAA |
| AA | | LGCLVKDYFPEPVTVSWNSGALTSGVHTFPAVLQSS |
| | | GLYSLSSVVTVPSSSLGTQTYICNVNHKPSNTKVDK |
| | | KVEPKSCDKTHTCPPCPAPPAAGPSVFLFPPKPKDT |
| | | LMISRTPEVTCVVVDVSHEDPEVKFNWYVDGVEVHN |
| | | AKTKPREEQYNSTYRVVSVLTVLHQDWLNGKEYKCK |
| | | VSNKALAAPIEKTISKAKGQPREPQVYTLPPSREEM |
| | | TKNQVSLTCLVKGFYPSDIAVEWESNGQPENNYKTT |
| | | PPVLDSDGSFFLYSKLTVDKSRWQQGNVFSCSVMHE |
| | | ALHNHYTQKSLSLSPGKGGGGSEVQLLESGGGLVQP |
| | | GGSLRLSCAASGFTVSAEDVGWVRQAPGKGLEWVSA |
| | | ILDKGGSTYYAESVKGRFTISRDNAKNTLYLQMSSL |
| | | RAEDTAVYYCARIVYHAGGGVTFDTRGQGTQVTVSS |
| | | GGGGSEVQLLESGGGLVQPGGSLRLSCAASGFTVSA |
| | | EDVGWVRQAPGKGLEWVSAILDKGGSTYYAESVKGR |
| | | FTISRDNAKNTLYLQMSSLRAEDTAVYYCARIVYHA |
| | | GGGVTFDTRGQGTQVTVSS |
| BE-718 | SEQ ID NO: 296 | CAGGTGCAGCTGGTGCAGAGCGGCGCGGAAGTGAAA |
| A-BGA-5623- | | AAACCGGGCGCGAGCGTGAAAGTGAGCTGCAAAGCG |
| BGA-5623 | | AGCGGCTATATTTTTACCAGCTATTACCTGCATTGG |
| heavy chain | | GTGCGCCAGGCGCCGGGCCAGGGCCTGGAATGGATT |
| DNA | | GGCTATATTAACCCGCAGACCGGCAAGACCAGCTAT |
| | | GCCCAGAAATTTCAGGGCCGCGTGACCATGACCCGC |
| | | GATACCAGCACCAGCACCGTGTATATGGAACTGAGC |
| | | AGCCTGCGCAGCGAAGATACCGCGGTGTATTATTGC |
| | | GCGCGCGAATATGGCAACTATAACTATCCGCTGGAT |
| | | TATTGGGGCCAGGGCACCCTGGTGACCGTGAGCAGC |
| | | GCTAGCACCAAGGGGCCCTCGGTCTTCCCCCTGGCA |
| | | CCCTCCTCCAAGAGTACTTCTGGGGGCACAGCGGCC |
| | | CTGGGCTGCCTGGTCAAGGACTACTTCCCCGAACCG |
| | | GTGACGGTGTCGTGGAACTCAGGCGCCCTGACCAGC |
| | | GGCGTGCACACCTTCCCGGCTGTCCTACAGTCCTCA |
| | | GGACTCTACTCCCTCAGCAGCGTGGTGACCGTGCCC |
| | | TCCAGCAGCTTGGGCACCCAGACCTACATCTGCAAC |
| | | GTGAATCACAAGCCCAGCAACACCAAGGTGGACAAG |
| | | AAAGTTGAGCCAAAGTCCTGTGACAAGACCCACACA |
| | | TGCCCCCCTTGTCCTGCTCCACCAGCTGCAGGACCA |
| | | AGCGTGTTCCTGTTTCCACCCAAGCCCAAGGATACC |
| | | CTGATGATCTCTCGGACCCCAGAGGTGACATGCGTG |
| | | GTGGTGGATGTGAGCCACGAGGACCCCGAGGTGAAG |
| | | TTCAACTGGTATGTGGACGGCGTGGAGGTGCACAAT |
| | | GCTAAGACCAAGCCCAGGGAGGAGCAGTACAACTCC |
| | | ACCTATAGAGTGGTGTCTGTGCTGACAGTGCTGCAC |
| | | CAGGATTGGCTGAACGGCAAGGAGTATAAGTGCAAG |
| | | GTGTCCAATAAGGCCCTGGCCGCTCCTATCGAGAAG |
| | | ACCATCTCTAAGGCCAAGGGCCAGCCCAGAGAGCCT |
| | | CAGGTGTACACACTGCCTCCATCCCGGGAAGAGATG |
| | | ACCAAGAACCAGGTGTCTCTGACATGTCTGGTCAAG |
| | | GGCTTCTATCCCTCTGACATCGCCGTGGAGTGGGAG |
| | | AGCAATGGCCAGCCTGAGAACAATTACAAGACCACA |
| | | CCCCCTGTGCTGGATTCCGACGGCTCTTTCTTTCTG |
| | | TATAGCAAGCTGACCGTGGACAAGTCCCGGTGGCAG |
| | | CAGGGCAACGTGTTCAGCTGTTCCGTGATGCACGAA |
| | | GCTCTGCATAATCACTATACTCAGAAATCCCTGTCA |
| | | CTGTCACCTGGTAAAGGTGGAGGCGGTTCAGAGGTC |
| | | CAGTTACTTGAGAGTGGTGGAGGTCTGGTCCAACCA |
| | | GGAGGTTCGCTGCGTTTATCCTGCGCCGCGTCTGGA |
| | | TTCACGGTTTCCGCCGAAGACGTGGGTTGGGTGCGT |
| | | CAAGCGCCGGGGAAAGGACTGGAATGGGTCTCCGCC |
| | | ATCTTGGATAAGGGTGGTTCGACATACTATGCGGAA |
| | | AGTGTCAAAGGGCGCTTTACGATCTCGCGCGATAAC |
| | | GCAAAAAATACTCTTTACCTTCAAATGTCTAGCCTT |
| | | CGTGCTGAGGACACTGCGGTGTACTACTGCGCCCGC |
| | | ATTGTCTACCATGCTGGTGGTGGCGTCACCTTTGAT |
| | | ACTCGGGGACAGGGCACCCAAGTTACAGTCTCATCG |
| | | GGCGGCGGAGGATCGGAGGTCCAGTTACTTGAGAGT |
| | | GGTGGAGGTCTGGTCCAACCAGGAGGTTCGCTGCGT |
| | | TTATCCTGCGCCGCGTCTGGATTCACGGTTTCCGCC |
| | | GAAGACGTGGGTTGGGTGCGTCAAGCGCCGGGGAAA |
| | | GGACTGGAATGGGTCTCCGCCATCTTGGATAAGGGT |
| | | GGTTCGACATACTATGCGGAAAGTGTCAAAGGGCGC |
| | | TTTACGATCTCGCGCGATAACGCAAAAAATACTCTT |
| | | TACCTTCAAATGTCTAGCCTTCGTGCTGAGGACACT |
| | | GCGGTGTACTACTGCGCCCGCATTGTCTACCATGCT |
| | | GGTGGTGGCGTCACCTTTGATACTCGGGGACAGGGC |
| | | ACCCAAGTTACAGTCTCATCG |
| BE-740 AA | SEQ ID NO: 297 | QVQLVQSGAEVKKPGASVKVSCKASGYIFTSYYLHW |
| | | VRQAPGQGLEWIGYINPQTGKTSYAQKFQGRVTMTR |
| | | DTSTSTVYMELSSLRSEDTAVYYCAREYGNYNYPLD |
| | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDK |
| | | KVEPKSCDKTHTCPPCPAPELLGGPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYKC |
| | | KVSNKALPAPIEKTISKAKGQPREPQVYTLPPSRDE |
| | | LTKNQVSLTCLVKGFYPSDIAVEWESNGQPENNYKT |
| | | TPPVLDSDGSFFLYSKLTVDKSRWQQGNVFSCSVMH |
| | | EALHNHYTQKSLSLSPGKGGGGSEVQLLESGGGLVQ |
| | | PGGSLRLSCAASGFTVSAEDVGWVRQAPGKGLEWVS |
| | | AILDKGGSTYYAESVKGRFTISRDNAKNTLYLQMSS |
| | | LRAEDTAVYYCARIVYHAGGGVTFDTRGQGTQVTVS |
| | | S |
| BE-740 DNA | SEQ ID NO: 298 | CAGGTGCAGCTGGTGCAGAGCGGCGCGGAAGTGAAA |
| | | AAACCGGGCGCGAGCGTGAAAGTGAGCTGCAAAGCG |
| | | AGCGGCTATATTTTTACCAGCTATTACCTGCATTGG |
| | | GTGCGCCAGGCGCCGGGCCAGGGCCTGGAATGGATT |
| | | GGCTATATTAACCCGCAGACCGGCAAGACCAGCTAT |
| | | GCCCAGAAATTTCAGGGCCGCGTGACCATGACCCGC |
| | | GATACCAGCACCAGCACCGTGTATATGGAACTGAGC |
| | | AGCCTGCGCAGCGAAGATACCGCGGTGTATTATTGC |
| | | GCGCGCGAATATGGCAACTATAACTATCCGCTGGAT |
| | | TATTGGGGCCAGGGCACCCTGGTGACCGTGAGCAGC |
| | | GCTAGCACCAAGGGGCCCTCGGTCTTCCCCCTGGCA |
| | | CCCTCCTCCAAGAGTACTTCTGGGGGCACAGCGGCC |
| | | CTGGGCTGCCTGGTCAAGGACTACTTCCCCGAACCG |
| | | GTGACGGTGTCGTGGAACTCAGGCGCCCTGACCAGC |
| | | GGCGTGCACACCTTCCCGGCTGTCCTACAGTCCTCA |
| | | GGACTCTACTCCCTCAGCAGCGTGGTGACCGTGCCC |
| | | TCCAGCAGCTTGGGCACCCAGACCTACATCTGCAAC |
| | | GTGAATCACAAGCCCAGCAACACCAAGGTGGACAAG |
| | | AAAGTTGAGCCCAAATCTTGTGACAAAACTCACACA |
| | | TGCCCACCGTGCCCAGCACCTGAACTCCTGGGGGGA |
| | | CCGTCAGTCTTCCTCTTCCCCCCAAAACCCAAGGAC |
| | | ACCCTCATGATCTCCCGGACCCCTGAGGTCACATGC |
| | | GTGGTGGTGGACGTGAGCCACGAAGACCCTGAGGTC |
| | | AAGTTCAACTGGTACGTGGACGGCGTGGAGGTGCAT |
| | | AATGCCAAGACAAAGCCGCGGGAGGAGCAGTACAAC |
| | | AGCACGTACCGTGTGGTCAGCGTCCTCACCGTCCTG |
| | | CACCAGGACTGGCTGAATGGCAAGGAGTACAAGTGC |
| | | AAGGTCTCCAACAAAGCCCTCCCAGCCCCCATCGAG |
| | | AAAACCATCTCCAAAGCCAAAGGGCAGCCCCGAGAA |
| | | CCACAGGTGTACACCCTGCCCCCATCCCGGGATGAG |
| | | CTGACCAAGAACCAGGTCAGCCTGACCTGCCTGGTC |
| | | AAAGGCTTCTATCCCAGCGACATCGCCGTGGAGTGG |
| | | GAGAGCAATGGGCAGCCGGAGAACAACTACAAGACC |
| | | ACGCCTCCCGTGCTGGACTCCGACGGCTCCTTCTTC |
| | | CTCTACAGCAAGCTCACCGTGGACAAGAGCAGGTGG |
| | | CAGCAGGGGAACGTCTTCTCATGCTCCGTGATGCAT |
| | | GAGGCTCTGCACAACCACTACACGCAGAAGAGCCTC |
| | | TCCCTGTCTCCGGGTAAAGGTGGAGGCGGTTCAGAG |
| | | GTCCAGTTACTTGAGAGTGGTGGAGGTCTGGTCCAA |
| | | CCAGGAGGTTCGCTGCGTTTATCCTGCGCCGCGTCT |
| | | GGATTCACGGTTTCCGCCGAAGACGTGGGTTGGGTG |
| | | CGTCAAGCGCCGGGGAAAGGACTGGAATGGGTCTCC |
| | | GCCATCTTGGATAAGGGTGGTTCGACATACTATGCG |
| | | GAAAGTGTCAAAGGGCGCTTTACGATCTCGCGCGAT |
| | | AACGCAAAAAATACTCTTTACCTTCAAATGTCTAGC |
| | | CTTCGTGCTGAGGACACTGCGGTGTACTACTGCGCC |
| | | CGCATTGTCTACCATGCTGGTGGTGGCGTCACCTTT |
| | | GATACTCGGGGACAGGGCACCCAAGTTACAGTCTCA |
| | | TCG |
| BE-942 | SEQ ID NO: 299 | QVQLVQSGAEVKKPGASVKVSCKASGYIFTSYYLHW |
| (ZW1 + 1) anti- | | VRQAPGQGLEWIGYINPQTGKTSYAQKFQGRVTMTR |
| CEA heavy | | DTSTSTVYMELSSLRSEDTAVYYCAREYGNYNYPLD |
| chain AA | | YWGQGTLVTVSSASTKGPSVFPLAPSSKSTSGGTAW |
| | | LGCEVTDYFPEPVTVSWNSGALTSGVHTFPAVLESS |
| | | GLYSLSSVVTVPSSSLGTQTYICNVNHKPSNTKVDK |
| | | KVEPKSCDKTHTCPPCPAPEAAGGPSVFLFPPKPKD |
| | | TLMISRTPEVTCVVVSVSHEDPEVKFNWYVDGVEVH |
| | | NAKTKPREEQYNSTYRVVSVLTVLHQDWLNGKEYKC |
| | | KVSNKALPAPIEKTISKAKGQPREPQVYVLPPSRDE |
| | | LTKNQVSLLCLVKGFYPSDIAVEWESNGQPENNYLT |
| | | WPPVLDSDGSFFLYSKLTVDKSRWQQGNVFSCSVMH |
| | | EALHNHYTQKSLSLSPGK |
| BE-942 | SEQ ID NO: 300 | CAGGTGCAGCTGGTGCAGAGCGGCGCGGAAGTGAAA |
| (ZW1 + 1) anti- | | AAACCGGGCGCGAGCGTGAAAGTGAGCTGCAAAGCG |
| CEA heavy | | AGCGGCTATATTTTTACCAGCTATTACCTGCATTGG |
| chain DNA | | GTGCGCCAGGCGCCGGGCCAGGGCCTGGAATGGATT |
| | | GGCTATATTAACCCGCAGACCGGCAAGACCAGCTAT |
| | | GCCCAGAAATTTCAGGGCCGCGTGACCATGACCCGC |
| | | GATACCAGCACCAGCACCGTGTATATGGAACTGAGC |
| | | AGCCTGCGCAGCGAAGATACCGCGGTGTATTATTGC |
| | | GCGCGCGAATATGGCAACTATAACTATCCGCTGGAT |
| | | TATTGGGGCCAGGGCACCCTGGTGACCGTGAGCAGC |
| | | GCTAGCACCAAGGGACCAAGCGTGTTCCCACTGGCA |
| | | CCCAGCTCCAAGAGCACCTCCGGAGGAACAGCATGG |
| | | CTGGGATGCGAGGTGACCGACTACTTCCCCGAGCCT |
| | | GTGACAGTGTCTTGGAACAGCGGCGCCCTGACCAGC |
| | | GGAGTGCACACATTTCCTGCCGTGCTGGAGTCTAGC |
| | | GGCCTGTACTCCCTGTCCTCTGTGGTGACCGTGCCT |
| | | AGCTCCTCTCTGGGCACCCAGACATATATCTGTAAC |
| | | GTGAATCACAAGCCATCTAATACAAAGGTGGATAAG |
| | | AAGGTGGAGCCAAAGTCCTGCGACAAGACCCACACA |
| | | TGCCCACCTTGTCCAGCACCAGAGGCAGCAGGAGGA |
| | | CCATCCGTGTTCCTGTTTCCACCCAAGCCCAAGGAT |
| | | ACCCTGATGATCTCTCGGACCCCCGAGGTGACATGC |
| | | GTGGTGGTGAGCGTGTCCCACGAGGACCCTGAGGTG |
| | | AAGTTCAACTGGTACGTGGATGGCGTGGAGGTGCAC |
| | | AATGCCAAGACAAAGCCCCGGGAGGAGCAGTACAAC |
| | | AGCACCTATAGAGTGGTGTCCGTGCTGACAGTGCTG |
| | | CACCAGGACTGGCTGAACGGCAAGGAGTATAAGTGC |
| | | AAGGTGTCTAATAAGGCCCTGCCTGCCCCAATCGAG |
| | | AAGACCATCAGCAAGGCAAAGGGACAGCCCAGGGAG |
| | | CCTCAGGTGTACGTGCTGCCTCCAAGCCGCGACGAG |
| | | CTGACAAAGAACCAGGTGTCCCTGCTGTGCCTGGTG |
| | | AAGGGCTTCTATCCTTCCGATATCGCCGTGGAGTGG |
| | | GAGTCTAATGGCCAGCCAGAGAACAATTACCTGACC |
| | | TGGCCCCCTGTGCTGGACTCCGATGGCTCTTTCTTT |
| | | CTGTATTCTAAGCTGACAGTGGATAAGAGCAGGTGG |
| | | CAGCAGGGCAACGTGTTTTCTTGCAGCGTGATGCAC |
| | | GAGGCCCTGCACAATCACTACACCCAGAAGTCCCTG |
| | | TCTCTGAGCCCTGGCAAG |
| BE-942 | SEQ ID NO: 301 | DIQMTQSPSSLSASVGDRVTITCRASENQYGYLAWY |
| (ZW1 + 1) anti- | | QQKPGKVPKLLIYNYKNLVEGVPSRFSGSGSGTDFT |
| CEA light | | LTISSLQPEDVATYYCQHHLGTPYTFGQGTKVEIKR |
| chain AA | | TVAAPSVAIFPPSDERLKSGTASVVCVLNNFYPREA |
| | | KVQWKVDNALQSGNSQESVTEQDSKDSTYSLSSRLT |
| | | LSKADYEKHKVYACEVTHQGLSSPVTKSFNRGEC |
| BE-942 | SEQ ID NO: 302 | GATATTCAGATGACCCAGAGCCCGAGCAGCCTGAGC |
| (ZW1 + 1) anti- | | GCGAGCGTGGGCGATCGCGTGACCATTACCTGCCGC |
| CEA light | | GCGAGCGAAAACCAGTATGGCTATCTGGCGTGGTAT |
| chain DNA | | CAGCAGAAACCGGGCAAAGTGCCGAAACTGCTGATT |
| | | TATAACTATAAAAACCTGGTGGAAGGCGTGCCGAGC |
| | | CGCTTTAGCGGCAGCGGCAGCGGCACCGATTTTACC |
| | | CTGACCATTAGCAGCCTGCAGCCGGAAGATGTGGCG |
| | | ACCTATTATTGCCAGCATCATCTGGGCACCCCGTAT |
| | | ACCTTTGGCCAGGGCACCAAAGTGGAAATTAAAAGG |
| | | ACAGTGGCAGCACCAAGCGTGGCAATCTTCCCACCT |
| | | TCCGACGAGAGACTGAAGTCCGGCACCGCTAGCGTG |
| | | GTGTGCGTGCTGAACAACTTCTACCCCCGGGAGGCC |
| | | AAGGTGCAGTGGAAGGTGGATAACGCCCTGCAGTCC |
| | | GGCAATTCTCAGGAGAGCGTGACCGAGCAGGACTCC |
| | | AAGGATTCTACATATAGCCTGAGCTCCAGGCTGACC |
| | | CTGTCTAAGGCCGACTACGAGAAGCACAAGGTGTAT |
| | | GCCTGCGAGGTGACACACCAGGGCCTGTCTAGCCCC |
| | | GTGACCAAGTCCTTCAACCGCGGCGAGTGT |
| BE-942 | SEQ ID NO: 303 | EVQLLESGGGLVQPGGSLRLSCAASGFTVSAEDVGW |
| (ZW1 + 1) anti- | | VRQAPGKGLEWVSAILDKGGSTYYAESVKGRFTISR |
| CD137 heavy | | DNAKNTLYLQMSSLRAEDTAVYYCARIVYHAGGGVT |
| chain AA | | FDTRGQGTQVTVSSEPKSSDKTHTCPPCPAPEAAGG |
| | | PSVFLFPPKPKDTLMISRTPEVTCVVVSVSHEDPEV |
| | | KFNWYVDGVEVHNAKTKPREEQYNSTYRVVSVLTVL |
| | | HQDWLNGKEYKCKVSNKALPAPIEKTISKAKGQPRE |
| | | PQVYVYPPSRDELTKNQVSLTCLVKGFYPSDIAVEW |
| | | ESNGQPENNYKTTPPVLDSDGSFALVSKLTVDKSRW |
| | | QQGNVFSCSVMHEALHNHYTQKSLSLSPGK |
| BE-942 | SEQ ID NO: 304 | GAGGTCCAGTTACTTGAGAGTGGTGGAGGTCTGGTC |
| (ZW1 + 1) anti- | | CAACCAGGAGGTTCGCTGCGTTTATCCTGCGCCGCG |
| CD137 heavy | | TCTGGATTCACGGTTTCCGCCGAAGACGTGGGTTGG |
| chain DNA | | GTGCGTCAAGCGCCGGGGAAAGGACTGGAATGGGTC |
| | | TCCGCCATCTTGGATAAGGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCATTGTCTACCATGCTGGTGGTGGCGTCACC |
| | | TTTGATACTCGGGGACAGGGCACCCAAGTTACAGTC |
| | | TCATCGGAGCCAAAGTCCTCCGACAAGACCCACACA |
| | | TGCCCACCTTGTCCAGCACCAGAGGCAGCAGGAGGA |
| | | CCATCCGTGTTCCTGTTTCCACCCAAGCCCAAGGAT |
| | | ACCCTGATGATCTCTCGGACCCCCGAGGTGACATGC |
| | | GTGGTGGTGAGCGTGTCCCACGAGGACCCTGAGGTG |
| | | AAGTTCAACTGGTACGTGGATGGCGTGGAGGTGCAC |
| | | AATGCCAAGACAAAGCCCCGGGAGGAGCAGTACAAC |
| | | AGCACCTATAGAGTGGTGTCCGTGCTGACAGTGCTG |
| | | CACCAGGACTGGCTGAACGGCAAGGAGTATAAGTGC |
| | | AAGGTGTCTAATAAGGCCCTGCCTGCCCCAATCGAG |
| | | AAGACCATCAGCAAGGCAAAGGGACAGCCCAGGGAG |
| | | CCTCAGGTGTACGTGTATCCTCCAAGCCGCGACGAG |
| | | CTGACCAAGAACCAGGTGTCCCTGACATGTCTGGTG |
| | | AAGGGCTTTTACCCTTCCGATATCGCCGTGGAGTGG |
| | | GAGTCTAATGGCCAGCCAGAGAACAATTATAAGACC |
| | | ACACCCCCTGTGCTGGACTCCGATGGCTCTTTCGCC |
| | | CTGGTGTCTAAGCTGACCGTGGATAAGAGCAGGTGG |
| | | CAGCAGGGCAACGTGTTTTCTTGCAGCGTGATGCAC |
| | | GAGGCCCTGCACAATCACTACACACAGAAGTCCCTG |
| | | TCTCTGAGCCCTGGCAAG |
| BE-755 | SEQ ID NO: 305 | EVQLLESGGGLVQPGGSLRLSCAASGFTVSAEDVGW |
| (ZW1 + 2) anti- | | VRQAPGKGLEWVSAILDKGGSTYYAESVKGRFTISR |
| CD137 heavy | | DNAKNTLYLQMSSLRAEDTAVYYCARIVYHAGGGVT |
| chain AA | | FDTRGQGTQVTVSSGGGGSEVQLLESGGGLVQPGGS |
| | | LRLSCAASGFTVSAEDVGWVRQAPGKGLEWVSAILD |
| | | KGGSTYYAESVKGRFTISRDNAKNTLYLQMSSLRAE |
| | | DTAVYYCARIVYHAGGGVTFDTRGQGTQVTVSSEPK |
| | | SSDKTHTCPPCPAPEAAGGPSVFLFPPKPKDTLMIS |
| | | RTPEVTCVVVSVSHEDPEVKFNWYVDGVEVHNAKTK |
| | | PREEQYNSTYRVVSVLTVLHQDWLNGKEYKCKVSNK |
| | | ALPAPIEKTISKAKGQPREPQVYVYPPSRDELTKNQ |
| | | VSLTCLVKGFYPSDIAVEWESNGQPENNYKTTPPVL |
| | | DSDGSFALVSKLTVDKSRWQQGNVFSCSVMHEALHN |
| | | HYTQKSLSLSPGK |
| BE-755 | SEQ ID NO: 306 | GAGGTCCAGTTACTTGAGAGTGGTGGAGGTCTGGTC |
| (ZW1 + 2) anti- | | CAACCAGGAGGTTCGCTGCGTTTATCCTGCGCCGCG |
| CD137 heavy | | TCTGGATTCACGGTTTCCGCCGAAGACGTGGGTTGG |
| chain DNA | | GTGCGTCAAGCGCCGGGGAAAGGACTGGAATGGGTC |
| | | TCCGCCATCTTGGATAAGGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCATTGTCTACCATGCTGGTGGTGGCGTCACC |
| | | TTTGATACTCGGGGACAGGGCACCCAAGTTACAGTC |
| | | TCATCGGGCGGCGGAGGGAGCGAGGTGCAGCTGCTT |
| | | GAGAGTGGTGGAGGTCTGGTCCAACCAGGAGGTTCG |
| | | CTGCGTTTATCCTGCGCCGCGTCTGGATTCACGGTT |
| | | TCCGCCGAAGACGTGGGTTGGGTGCGTCAAGCGCCG |
| | | GGGAAAGGACTGGAATGGGTCTCCGCCATCTTGGAT |
| | | AAGGGTGGTTCGACATACTATGCGGAAAGTGTCAAA |
| | | GGGCGCTTTACGATCTCGCGCGATAACGCAAAAAAT |
| | | ACTCTTTACCTTCAAATGTCTAGCCTTCGTGCTGAG |
| | | GACACTGCGGTGTACTACTGCGCCCGCATTGTCTAC |
| | | CATGCTGGTGGTGGCGTCACCTTTGATACTCGGGGA |
| | | CAGGGCACCCAAGTGACCGTGTCCAGCGAGCCAAAG |
| | | TCCTCCGACAAGACCCACACATGCCCACCTTGTCCA |
| | | GCACCAGAGGCAGCAGGAGGACCATCCGTGTTCCTG |
| | | TTTCCACCCAAGCCCAAGGATACCCTGATGATCTCT |
| | | CGGACCCCCGAGGTGACATGCGTGGTGGTGAGCGTG |
| | | TCCCACGAGGACCCTGAGGTGAAGTTCAACTGGTAC |
| | | GTGGATGGCGTGGAGGTGCACAATGCCAAGACAAAG |
| | | CCCCGGGAGGAGCAGTACAACAGCACCTATAGAGTG |
| | | GTGTCCGTGCTGACAGTGCTGCACCAGGACTGGCTG |
| | | AACGGCAAGGAGTATAAGTGCAAGGTGTCTAATAAG |
| | | GCCCTGCCTGCCCCAATCGAGAAGACCATCAGCAAG |
| | | GCAAAGGGACAGCCCAGGGAGCCTCAGGTGTACGTG |
| | | TATCCTCCAAGCCGCGACGAGCTGACCAAGAACCAG |
| | | GTGTCCCTGACATGTCTGGTGAAGGGCTTTTACCCT |
| | | TCCGATATCGCCGTGGAGTGGGAGTCTAATGGCCAG |
| | | CCAGAGAACAATTATAAGACCACACCCCCTGTGCTG |
| | | GACTCCGATGGCTCTTTCGCCCTGGTGTCTAAGCTG |
| | | ACCGTGGATAAGAGCAGGTGGCAGCAGGGCAACGTG |
| | | TTTTCTTGCAGCGTGATGCACGAGGCCCTGCACAAT |
| | | CACTACACACAGAAGTCCCTGTCTCTGAGCCCTGGC |
| | | AAG |
| BE-562 | SEQ ID NO: 307 | EVQLLESGGGLVQPGGSLRLSCAASGFTVSAEDVGW |
| (E-mutFc) | | VRQAPGKGLEWVSAILDKGGSTYYAESVKGRFTISR |
| heavy | | DNAKNTLYLQMSSLRAEDTAVYYCARIVYHAGGGVT |
| chain AA | | FDTRGQGTQVTVSSEPKSSDKTHTCPPCPAPPAAGP |
| | | SVFLFPPKPKDTLMISRTPEVTCVVVDVSHEDPEVK |
| | | FNWYVDGVEVHNAKTKPREEQYNSTYRVVSVLTVLH |
| | | QDWLNGKEYKCKVSNKALAAPIEKTISKAKGQPREP |
| | | QVYTLPPSREEMTKNQVSLTCLVKGFYPSDIAVEWE |
| | | SNGQPENNYKTTPPVLDSDGSFFLYSKLTVDKSRWQ |
| | | QGNVFSCSVMHEALHNHYTQKSLSLSPGKGGGGSQV |
| | | QLVQSGAEVKKPGASVKVSCKASGYIFTSYYLHWVR |
| | | QAPGQGLEWIGYINPQTGKTSYAQKFQGRVTMTRDT |
| | | STSTVYMELSSLRSEDTAVYYCAREYGNYNYPLDYW |
| | | GQGTLVTVSSASTKGPSVFPLAPSSKSTSGGTAALG |
| | | CLVKDYFPEPVTVSWNSGALTSGVHTFPAVLQSSGL |
| | | YSLSSVVTVPSSSLGTQTYICNVNHKPSNTKVDKKV |
| | | EPKSCD |
| BE-562 | SEQ ID NO: 308 | GAGGTCCAGTTACTTGAGAGTGGTGGAGGTCTGGTC |
| (E-mutFc) | | CAACCAGGAGGTTCGCTGCGTTTATCCTGCGCCGCG |
| heavy | | TCTGGATTCACGGTTTCCGCCGAAGACGTGGGTTGG |
| chain DNA | | GTGCGTCAAGCGCCGGGGAAAGGACTGGAATGGGTC |
| | | TCCGCCATCTTGGATAAGGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCATTGTCTACCATGCTGGTGGTGGCGTCACC |
| | | TTTGATACTCGGGGACAGGGCACCCAAGTTACAGTC |
| | | TCATCGGAGCCAAAGTCCTCTGACAAGACCCACACA |
| | | TGCCCCCCTTGTCCTGCTCCACCAGCTGCAGGACCA |
| | | AGCGTGTTCCTGTTTCCACCCAAGCCCAAGGATACC |
| | | CTGATGATCTCTCGGACCCCAGAGGTGACATGCGTG |
| | | GTGGTGGATGTGAGCCACGAGGACCCCGAGGTGAAG |
| | | TTCAACTGGTATGTGGACGGCGTGGAGGTGCACAAT |
| | | GCTAAGACCAAGCCCAGGGAGGAGCAGTACAACTCC |
| | | ACCTATAGAGTGGTGTCTGTGCTGACAGTGCTGCAC |
| | | CAGGATTGGCTGAACGGCAAGGAGTATAAGTGCAAG |
| | | GTGTCCAATAAGGCCCTGGCCGCTCCTATCGAGAAG |
| | | ACCATCTCTAAGGCCAAGGGCCAGCCCAGAGAGCCT |
| | | CAGGTGTACACACTGCCTCCATCCCGGGAAGAGATG |
| | | ACCAAGAACCAGGTGTCTCTGACATGTCTGGTCAAG |
| | | GGCTTCTATCCCTCTGACATCGCCGTGGAGTGGGAG |
| | | AGCAATGGCCAGCCTGAGAACAATTACAAGACCACA |
| | | CCCCCTGTGCTGGATTCCGACGGCTCTTTCTTTCTG |
| | | TATAGCAAGCTGACCGTGGACAAGTCCCGGTGGCAG |
| | | CAGGGCAACGTGTTCAGCTGTTCCGTGATGCACGAA |
| | | GCTCTGCATAATCACTATACTCAGAAATCCCTGTCA |
| | | CTGTCACCTGGTAAAGGTGGAGGCGGTTCACAGGTG |
| | | CAGCTGGTGCAGAGCGGCGCGGAAGTGAAAAAACCG |
| | | GGCGCGAGCGTGAAAGTGAGCTGCAAAGCGAGCGGC |
| | | TATATTTTTACCAGCTATTACCTGCATTGGGTGCGC |
| | | CAGGCGCCGGGCCAGGGCCTGGAATGGATTGGCTAT |
| | | ATTAACCCGCAGACCGGCAAGACCAGCTATGCCCAG |
| | | AAATTTCAGGGCCGCGTGACCATGACCCGCGATACC |
| | | AGCACCAGCACCGTGTATATGGAACTGAGCAGCCTG |
| | | CGCAGCGAAGATACCGCGGTGTATTATTGCGCGCGC |
| | | GAATATGGCAACTATAACTATCCGCTGGATTATTGG |
| | | GGCCAGGGCACCCTGGTGACCGTGAGCAGCGCTAGC |
| | | ACCAAGGGGCCCTCGGTCTTCCCCCTGGCACCCTCC |
| | | TCCAAGAGTACTTCTGGGGGCACAGCGGCCCTGGGC |
| | | TGCCTGGTCAAGGACTACTTCCCCGAACCGGTGACG |
| | | GTGTCGTGGAACTCAGGCGCCCTGACCAGCGGCGTG |
| | | CACACCTTCCCGGCTGTCCTACAGTCCTCAGGACTC |
| | | TACTCCCTCAGCAGCGTGGTGACCGTGCCCTCCAGC |
| | | AGCTTGGGCACCCAGACCTACATCTGCAACGTGAAT |
| | | CACAAGCCCAGCAACACCAAGGTGGACAAGAAAGTT |
| | | GAGCCCAAATCTTGTGAC |
| BE-375 | SEQ ID NO: 309 | EVQLLESGGGLVQPGGSLRLSCAASGFTVSAEDVGW |
| (E-wtIgG1 Fc) | | VRQAPGKGLEWVSAILDKGGSTYYAESVKGRFTISR |
| heavy chain | | DNAKNTLYLQMSSLRAEDTAVYYCARIVYHAGGGVT |
| AA | | FDTRGQGTQVTVSSEPKSSDKTHTCPPCPAPELLGG |
| | | PSVFLFPPKPKDTLMISRTPEVTCVVVDVSHEDPEV |
| | | KFNWYVDGVEVHNAKTKPREEQYNSTYRVVSVLTVL |
| | | HQDWLNGKEYKCKVSNKALPAPIEKTISKAKGQPRE |
| | | PQVYTLPPSRDELTKNQVSLTCLVKGFYPSDIAVEW |
| | | ESNGQPENNYKTTPPVLDSDGSFFLYSKLTVDKSRW |
| | | QQGNVFSCSVMHEALHNHYTQKSLSLSPGKGGGGSQ |
| | | VQLVQSGAEVKKPGASVKVSCKASGYIFTSYYLHWV |
| | | RQAPGQGLEWIGYINPQTGKTSYAQKFQGRVTMTRD |
| | | TSTSTVYMELSSLRSEDTAVYYCAREYGNYNYPLDY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKK |
| | | VEPKSCD |
| BE-375 | SEQ ID NO: 310 | GAGGTCCAGTTACTTGAGAGTGGTGGAGGTCTGGTC |
| (E-wtIgG1 Fc) | | CAACCAGGAGGTTCGCTGCGTTTATCCTGCGCCGCG |
| heavy chain | | TCTGGATTCACGGTTTCCGCCGAAGACGTGGGTTGG |
| DNA | | GTGCGTCAAGCGCCGGGGAAAGGACTGGAATGGGTC |
| | | TCCGCCATCTTGGATAAGGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCATTGTCTACCATGCTGGTGGTGGCGTCACC |
| | | TTTGATACTCGGGGACAGGGCACCCAAGTTACAGTC |
| | | TCATCGGAGCCCAAATCTTCTGACAAAACTCACACA |
| | | TGCCCACCGTGCCCAGCACCTGAACTCCTGGGGGGA |
| | | CCGTCAGTCTTCCTCTTCCCCCCAAAACCCAAGGAC |
| | | ACCCTCATGATCTCCCGGACCCCTGAGGTCACATGC |
| | | GTGGTGGTGGACGTGAGCCACGAAGACCCTGAGGTC |
| | | AAGTTCAACTGGTACGTGGACGGCGTGGAGGTGCAT |
| | | AATGCCAAGACAAAGCCGCGGGAGGAGCAGTACAAC |
| | | AGCACGTACCGTGTGGTCAGCGTCCTCACCGTCCTG |
| | | CACCAGGACTGGCTGAATGGCAAGGAGTACAAGTGC |
| | | AAGGTCTCCAACAAAGCCCTCCCAGCCCCCATCGAG |
| | | AAAACCATCTCCAAAGCCAAAGGGCAGCCCCGAGAA |
| | | CCACAGGTGTACACCCTGCCCCCATCCCGGGATGAG |
| | | CTGACCAAGAACCAGGTCAGCCTGACCTGCCTGGTC |
| | | AAAGGCTTCTATCCCAGCGACATCGCCGTGGAGTGG |
| | | GAGAGCAATGGGCAGCCGGAGAACAACTACAAGACC |
| | | ACGCCTCCCGTGCTGGACTCCGACGGCTCCTTCTTC |
| | | CTCTACAGCAAGCTCACCGTGGACAAGAGCAGGTGG |
| | | CAGCAGGGGAACGTCTTCTCATGCTCCGTGATGCAT |
| | | GAGGCTCTGCACAACCACTACACGCAGAAGAGCCTC |
| | | TCCCTGTCTCCGGGTAAAGGTGGAGGCGGTTCACAG |
| | | GTGCAGCTGGTGCAGAGCGGCGCGGAAGTGAAAAAA |
| | | CCGGGCGCGAGCGTGAAAGTGAGCTGCAAAGCGAGC |
| | | GGCTATATTTTTACCAGCTATTACCTGCATTGGGTG |
| | | CGCCAGGCGCCGGGCCAGGGCCTGGAATGGATTGGC |
| | | TATATTAACCCGCAGACCGGCAAGACCAGCTATGCC |
| | | CAGAAATTTCAGGGCCGCGTGACCATGACCCGCGAT |
| | | ACCAGCACCAGCACCGTGTATATGGAACTGAGCAGC |
| | | CTGCGCAGCGAAGATACCGCGGTGTATTATTGCGCG |
| | | CGCGAATATGGCAACTATAACTATCCGCTGGATTAT |
| | | TGGGGCCAGGGCACCCTGGTGACCGTGAGCAGCGCT |
| | | AGCACCAAGGGGCCCTCGGTCTTCCCCCTGGCACCC |
| | | TCCTCCAAGAGTACTTCTGGGGGCACAGCGGCCCTG |
| | | GGCTGCCTGGTCAAGGACTACTTCCCCGAACCGGTG |
| | | ACGGTGTCGTGGAACTCAGGCGCCCTGACCAGCGGC |
| | | GTGCACACCTTCCCGGCTGTCCTACAGTCCTCAGGA |
| | | CTCTACTCCCTCAGCAGCGTGGTGACCGTGCCCTCC |
| | | AGCAGCTTGGGCACCCAGACCTACATCTGCAACGTG |
| | | AATCACAAGCCCAGCAACACCAAGGTGGACAAGAAA |
| | | GTTGAGCCCAAATCTTGTGAC |
| BE-244 | SEQ ID NO: 311 | QVQLVQSGAEVKKPGASVKVSCKASGYIFTSYYLHW |
| (A-(G4S)3) | | VRQAPGQGLEWIGYINPQTGKTSYAQKFQGRVTMTR |
| heavy chain | | DTSTSTVYMELSSLRSEDTAVYYCAREYGNYNYPLD |
| AA | | YWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDK |
| | | KVEPKSCDKTHTCPPCPAPPAAGPSVFLFPPKPKDT |
| | | LMISRTPEVTCVVVDVSHEDPEVKFNWYVDGVEVHN |
| | | AKTKPREEQYNSTYRVVSVLTVLHQDWLNGKEYKCK |
| | | VSNKALAAPIEKTISKAKGQPREPQVYTLPPSREEM |
| | | TKNQVSLTCLVKGFYPSDIAVEWESNGQPENNYKTT |
| | | PPVLDSDGSFFLYSKLTVDKSRWQQGNVFSCSVMHE |
| | | ALHNHYTQKSLSLSPGKGGGGSGGGGSGGGGSEVQL |
| | | LESGGGLVQPGGSLRLSCAASGFTVSAEDVGWVRQA |
| | | PGKGLEWVSAILDKGGSTYYAESVKGRFTISRDNAK |
| | | NTLYLQMSSLRAEDTAVYYCARIVYHAGGGVTFDTR |
| | | GQGTQVTVSS |
| BE-244 | SEQ ID NO: 312 | CAGGTGCAGCTGGTGCAGAGCGGCGCGGAAGTGAAA |
| (A-(G4S)3) | | AAACCGGGCGCGAGCGTGAAAGTGAGCTGCAAAGCG |
| heavy chain | | AGCGGCTATATTTTTACCAGCTATTACCTGCATTGG |
| DNA | | GTGCGCCAGGCGCCGGGCCAGGGCCTGGAATGGATT |
| | | GGCTATATTAACCCGCAGACCGGCAAGACCAGCTAT |
| | | GCCCAGAAATTTCAGGGCCGCGTGACCATGACCCGC |
| | | GATACCAGCACCAGCACCGTGTATATGGAACTGAGC |
| | | AGCCTGCGCAGCGAAGATACCGCGGTGTATTATTGC |
| | | GCGCGCGAATATGGCAACTATAACTATCCGCTGGAT |
| | | TATTGGGGCCAGGGCACCCTGGTGACCGTGAGCAGC |
| | | GCTAGCACCAAGGGGCCCTCGGTCTTCCCCCTGGCA |
| | | CCCTCCTCCAAGAGTACTTCTGGGGGCACAGCGGCC |
| | | CTGGGCTGCCTGGTCAAGGACTACTTCCCCGAACCG |
| | | GTGACGGTGTCGTGGAACTCAGGCGCCCTGACCAGC |
| | | GGCGTGCACACCTTCCCGGCTGTCCTACAGTCCTCA |
| | | GGACTCTACTCCCTCAGCAGCGTGGTGACCGTGCCC |
| | | TCCAGCAGCTTGGGCACCCAGACCTACATCTGCAAC |
| | | GTGAATCACAAGCCCAGCAACACCAAGGTGGACAAG |
| | | AAAGTTGAGCCAAAGTCCTGTGACAAGACCCACACA |
| | | TGCCCCCCTTGTCCTGCTCCACCAGCTGCAGGACCA |
| | | AGCGTGTTCCTGTTTCCACCCAAGCCCAAGGATACC |
| | | CTGATGATCTCTCGGACCCCAGAGGTGACATGCGTG |
| | | GTGGTGGATGTGAGCCACGAGGACCCCGAGGTGAAG |
| | | TTCAACTGGTATGTGGACGGCGTGGAGGTGCACAAT |
| | | GCTAAGACCAAGCCCAGGGAGGAGCAGTACAACTCC |
| | | ACCTATAGAGTGGTGTCTGTGCTGACAGTGCTGCAC |
| | | CAGGATTGGCTGAACGGCAAGGAGTATAAGTGCAAG |
| | | GTGTCCAATAAGGCCCTGGCCGCTCCTATCGAGAAG |
| | | ACCATCTCTAAGGCCAAGGGCCAGCCCAGAGAGCCT |
| | | CAGGTGTACACACTGCCTCCATCCCGGGAAGAGATG |
| | | ACCAAGAACCAGGTGTCTCTGACATGTCTGGTCAAG |
| | | GGCTTCTATCCCTCTGACATCGCCGTGGAGTGGGAG |
| | | AGCAATGGCCAGCCTGAGAACAATTACAAGACCACA |
| | | CCCCCTGTGCTGGATTCCGACGGCTCTTTCTTTCTG |
| | | TATAGCAAGCTGACCGTGGACAAGTCCCGGTGGCAG |
| | | CAGGGCAACGTGTTCAGCTGTTCCGTGATGCACGAA |
| | | GCTCTGCATAATCACTATACTCAGAAATCCCTGTCA |
| | | CTGTCACCTGGTAAAGGTGGAGGCGGTTCAGGCGGC |
| | | GGAGGATCTGGTGGCGGTGGGTCCGAGGTCCAGTTA |
| | | CTTGAGAGTGGTGGAGGTCTGGTCCAACCAGGAGGT |
| | | TCGCTGCGTTTATCCTGCGCCGCGTCTGGATTCACG |
| | | GTTTCCGCCGAAGACGTGGGTTGGGTGCGTCAAGCG |
| | | CCGGGGAAAGGACTGGAATGGGTCTCCGCCATCTTG |
| | | GATAAGGGTGGTTCGACATACTATGCGGAAAGTGTC |
| | | AAAGGGCGCTTTACGATCTCGCGCGATAACGCAAAA |
| | | AATACTCTTTACCTTCAAATGTCTAGCCTTCGTGCT |
| | | GAGGACACTGCGGTGTACTACTGCGCCCGCATTGTC |
| | | TACCATGCTGGTGGTGGCGTCACCTTTGATACTCGG |
| | | GGACAGGGCACCCAAGTTACAGTCTCATCG |

### Example 28. In Vivo Efficacy of Single-Agent CEA×CD137 Multispecific Antibody

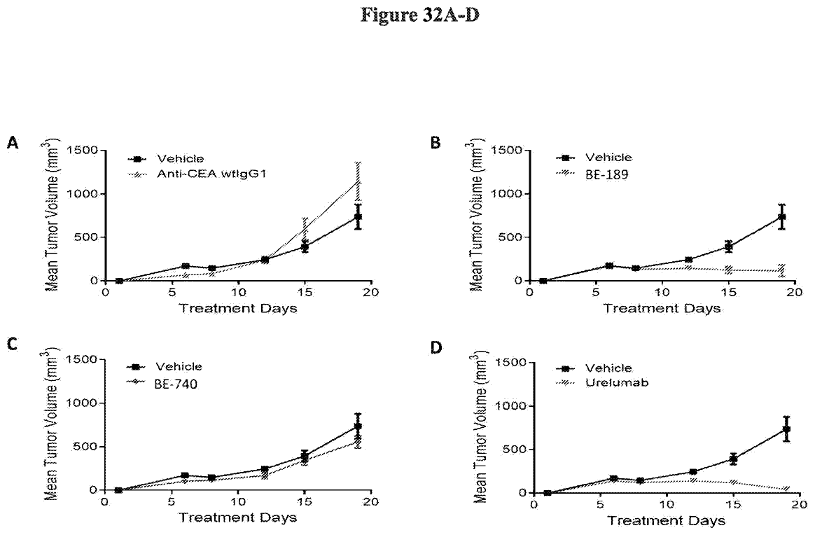

To determine the in vivo efficacy of CEA×CD137 multispecific antibodies BE-189 and BE-740 against CEA⁺ tumor cells, CT26/CEA cells (1×10⁶) were injected subcutaneously into humanized CD137 mice of the BALB/c background. BE-189 (3 mg/kg), BE-740 (3 mg/kg), anti-CEA Ab (SEQ ID NO: 181 and 179) (3 mg/kg), Urelumab analog (3 mg/kg) or vehicle control were given twice per week starting on the day of tumor injection (6 mice per group). As compared to vehicle control, BE-189 (A-BGA-5623) and Urelumab analog induced significant inhibition of tumor growth (P<0.001) as shown in FIG. 32.

### Example 29. CEA×CD137 Agonists

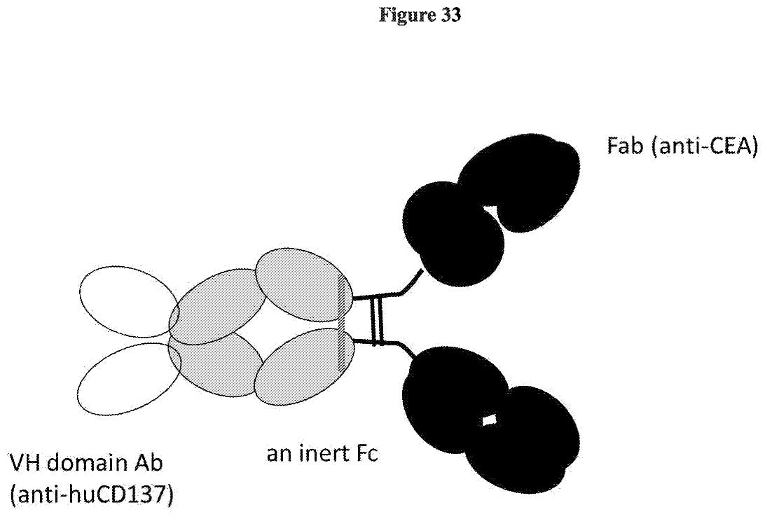

Agonistic anti-huCD137 antibodies have demonstrated toxicity in the clinical setting, which may indicate that systemic FcγR cross-linking is not ideal for CD137 activation. The aim was to achieve potent CD137 stimulation specifically at the tumor site without systemic CD137 activation for a broad range of cancers. To overcome the dependency of FcγR cross-linking, we generated a CEA×CD137 multispecific antibody with the following features as shown in FIG. 33. This specific construct included an IgG-fusion like multispecific antibody format with a module ratio of 2:2, a bivalent F(ab′)2 fragment that binds to CEA, VH domain fragments with a fusion at the C terminal of CH3, which bind huCD137, and a Fc null version of huIgG1, which has no FcγR binding but retain FcRn binding. The sequence information is shown in Table 37.

TABLE 37
Amino acid and DNA sequences of CEAxCD137

| Construct | SEQ ID NO | Sequence |
|---|---|---|
| BE-146 | SEQ ID NO: | QVQLVQSGAEVKKPGASVKVSCKASGYIFTSYYLHWVRQAP |
| heavy | 313 | GQGLEWIGYINPQTGKTSYAQKFQGRVTMTRDTSTSTVYME |
| chain AA | | LSSLRSEDTAVYYCAREYGNYNYPLDYWGQGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPPAAGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALAAPIEKTISKAKGQPREPQVYTLPPSREEMTKNQVSLTC |
| | | LVKGFYPSDIAVEWESNGQPENNYKTTPPVLDSDGSFFLYS |
| | | KLTVDKSRWQQGNVFSCSVMHEALHNHYTQKSLSLSPGKGG |
| | | GGSEVQLLESGGGLVQPGGSLRLSCAASGFTVSAEDVGWVR |
| | | QAPGKGLEWVSAILDKGGSTYYAESVKGRFTISRDNAKNTL |
| | | YLQMSSLRAEDTAVYYCARIVYHAGGGVTFDTRGQGTQVTV |
| | | SS |
| BE-146 | SEQ ID NO: | CAGGTGCAGCTGGTGCAGAGCGGCGCGGAAGTGAAAAAACC |
| heavy | 314 | GGGCGCGAGCGTGAAAGTGAGCTGCAAAGCGAGCGGCTATA |
| chain | | TTTTTACCAGCTATTACCTGCATTGGGTGCGCCAGGCGCCG |
| DNA | | GGCCAGGGCCTGGAATGGATTGGCTATATTAACCCGCAGAC |
| | | CGGCAAGACCAGCTATGCCCAGAAATTTCAGGGCCGCGTGA |
| | | CCATGACCCGCGATACCAGCACCAGCACCGTGTATATGGAA |
| | | CTGAGCAGCCTGCGCAGCGAAGATACCGCGGTGTATTATTG |
| | | CGCGCGCGAATATGGCAACTATAACTATCCGCTGGATTATT |
| | | GGGGCCAGGGCACCCTGGTGACCGTGAGCAGCGCTAGCACC |
| | | AAGGGGCCCTCGGTCTTCCCCCTGGCACCCTCCTCCAAGAG |
| | | TACTTCTGGGGGCACAGCGGCCCTGGGCTGCCTGGTCAAGG |
| | | ACTACTTCCCCGAACCGGTGACGGTGTCGTGGAACTCAGGC |
| | | GCCCTGACCAGCGGCGTGCACACCTTCCCGGCTGTCCTACA |
| | | GTCCTCAGGACTCTACTCCCTCAGCAGCGTGGTGACCGTGC |
| | | CCTCCAGCAGCTTGGGCACCCAGACCTACATCTGCAACGTG |
| | | AATCACAAGCCCAGCAACACCAAGGTGGACAAGAAAGTTGA |
| | | GCCAAAGTCCTGTGACAAGACCCACACATGCCCCCCTTGTC |
| | | CTGCTCCACCAGCTGCAGGACCAAGCGTGTTCCTGTTTCCA |
| | | CCCAAGCCCAAGGATACCCTGATGATCTCTCGGACCCCAGA |
| | | GGTGACATGCGTGGTGGTGGATGTGAGCCACGAGGACCCCG |
| | | AGGTGAAGTTCAACTGGTATGTGGACGGCGTGGAGGTGCAC |
| | | AATGCTAAGACCAAGCCCAGGGAGGAGCAGTACAACTCCAC |
| | | CTATAGAGTGGTGTCTGTGCTGACAGTGCTGCACCAGGATT |
| | | GGCTGAACGGCAAGGAGTATAAGTGCAAGGTGTCCAATAAG |
| | | GCCCTGGCCGCTCCTATCGAGAAGACCATCTCTAAGGCCAA |
| | | GGGCCAGCCCAGAGAGCCTCAGGTGTACACACTGCCTCCAT |
| | | CCCGGGAAGAGATGACCAAGAACCAGGTGTCTCTGACATGT |
| | | CTGGTCAAGGGCTTCTATCCCTCTGACATCGCCGTGGAGTG |
| | | GGAGAGCAATGGCCAGCCTGAGAACAATTACAAGACCACAC |
| | | CCCCTGTGCTGGATTCCGACGGCTCTTTCTTTCTGTATAGC |
| | | AAGCTGACCGTGGACAAGTCCCGGTGGCAGCAGGGCAACGT |
| | | GTTCAGCTGTTCCGTGATGCACGAAGCTCTGCATAATCACT |
| | | ATACTCAGAAATCCCTGTCACTGTCACCTGGTAAAGGTGGA |
| | | GGCGGTTCAGAGGTCCAGTTACTTGAGAGTGGTGGAGGTCT |
| | | GGTCCAACCAGGAGGTTCGCTGCGTTTATCCTGCGCCGCGT |
| | | CTGGATTCACGGTTTCCGCCGAAGACGTGGGTTGGGTGCGT |
| | | CAAGCGCCGGGGAAAGGACTGGAATGGGTCTCCGCCATCTT |
| | | GGATAAGGGTGGTTCGACATACTATGCGGAAAGTGTCAAAG |
| | | GGCGCTTTACGATCTCGCGCGATAACGCAAAAAATACTCTT |
| | | TACCTTCAAATGTCTAGCCTTCGTGCTGAGGACACTGCGGT |
| | | GTACTACTGCGCCCGCATTGTCTACCATGCTGGTGGTGGCG |
| | | TCACCTTTGATACTCGGGGACAGGGCACCCAAGTTACAGTC |
| | | TCATCG |
| BE-146 | SEQ ID NO: | DIQMTQSPSSLSASVGDRVTITCRASENQYGYLAWYQQKPG |
| light | 179 | KVPKLLIYNYKNLVEGVPSRFSGSGSGTDFTLTISSLQPED |
| chain AA | | VATYYCQHHLGTPYTFGQGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| BE-146 | SEQ ID NO: | GATATTCAGATGACCCAGAGCCCGAGCAGCCTGAGCGCGAG |
| light | 180 | CGTGGGCGATCGCGTGACCATTACCTGCCGCGCGAGCGAAA |
| chain | | ACCAGTATGGCTATCTGGCGTGGTATCAGCAGAAACCGGGC |
| DNA | | AAAGTGCCGAAACTGCTGATTTATAACTATAAAAACCTGGT |
| | | GGAAGGCGTGCCGAGCCGCTTTAGCGGCAGCGGCAGCGGCA |
| | | CCGATTTTACCCTGACCATTAGCAGCCTGCAGCCGGAAGAT |
| | | GTGGCGACCTATTATTGCCAGCATCATCTGGGCACCCCGTA |
| | | TACCTTTGGCCAGGGCACCAAAGTGGAAATTAAACGAACAG |
| | | TGGCAGCCCCTTCCGTCTTCATTTTTCCCCCTTCTGACGAA |
| | | CAGCTGAAATCAGGAACTGCTAGCGTGGTCTGTCTGCTGAA |
| | | CAATTTCTACCCCAGAGAGGCCAAGGTGCAGTGGAAAGTCG |
| | | ATAACGCTCTGCAGTCCGGCAATTCTCAGGAGAGTGTGACC |
| | | GAACAGGACTCAAAGGATAGCACATATTCCCTGTCTAGTAC |
| | | TCTGACCCTGAGCAAAGCAGACTACGAGAAGCACAAAGTGT |
| | | ATGCCTGTGAAGTCACACACCAGGGGCTGAGTTCACCAGTC |
| | | ACCAAGAGTTTCAACAGAGGGGAATGC |

### Example 30. Target-Binding Activity of CEA×CD137

### Binding to Recombinant CD137 and CEA

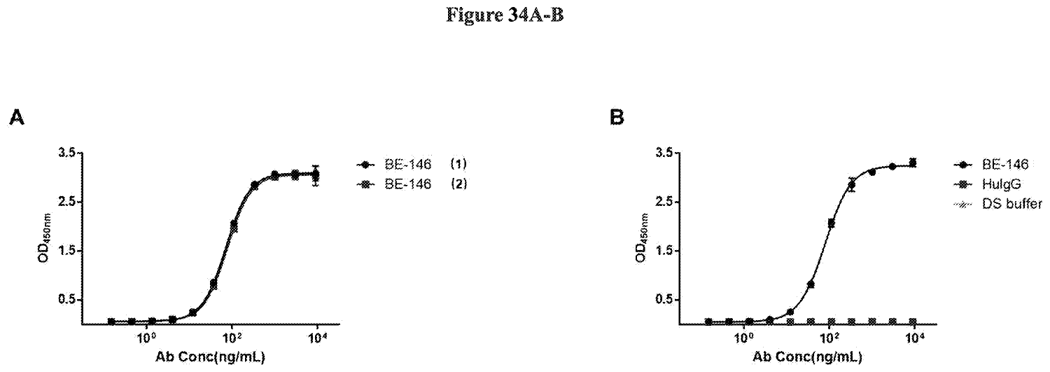

The ELISA results showed that BE-146 bound to the antigen CEA (CEA/His) and CD137 (huCD137-mIgG2a) with consistent binding activity, while the 2 negative controls, human IgG and DS (drug substance) buffer, had no detectable binding to CEA and CD137 (FIG. 34).

The binding kinetics of the BE146 were measured using surface plasmon resonance (SPR). We used SPR to measure the on-rate constant (ka) and off-rate constant (kd) of the antibodies to recombinant proteins of CD137 and CEA, and then determined the affinity constant (K_{D}). The results showed that BE-146 had comparable binding affinity to human CD137 and human CEA.

Human CD137 protein has low sequence homology to murine CD137, with only 61.0% sequence identity. In contrast, CD137 is highly homologous to cynomolgus monkey CD137, with 95% sequence identity. To test the species specificity of BE-146 binding function, SPR binding studies were performed using human, cynomolgus monkey, and mouse CD137 as binding proteins. BE-146 displayed a high binding affinity to human CD137 with a K_{D} of about 36.2 nM. In comparison, the binding affinity of BE-146 to cynomolgus monkey CD137 with a similar K_{D} of about 15.9 nM. BE-146 had no detectable binding signaling to mouse CD137 in SPR assay as shown in Table 38.

The binding affinity tests of CEAs between human and cynomolgous monkey species indicated that BE-146 displayed a similar binding affinity to human CEA (K_{D}: about 3.00 nM) and monkey CEA (Cat.:CE5-C52H5, Acrobiosystem) (K_{D}: about 11.4 nM). This data is shown in Table 39 below. A sequence alignment of human and monkey CEA indicates that there is 79.2% homology between the two species.

### Binding to Native CD137 and CEA

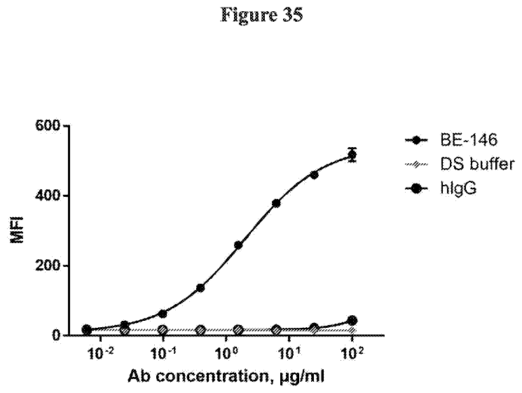

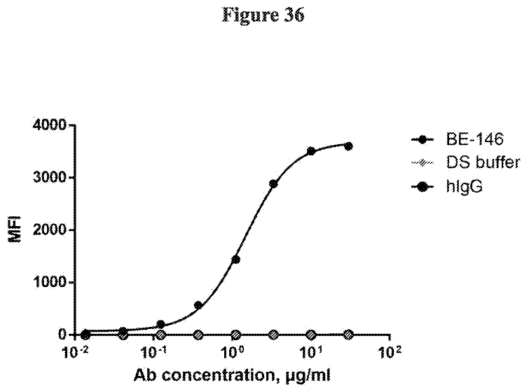

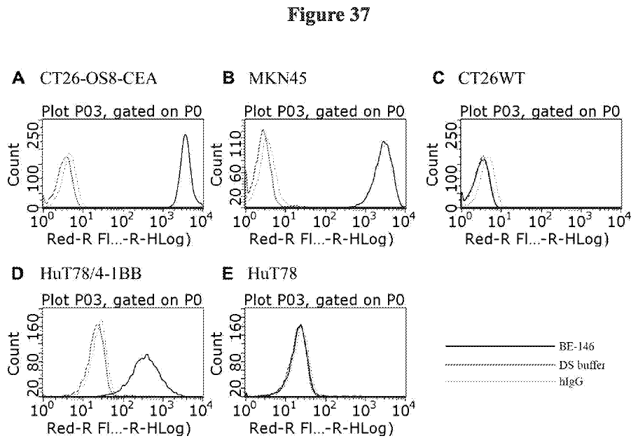

The FACS results further confirmed the binding activity of BE-146 to CD137 expressed on HuT78/CD137 cell surface. BE-146 showed strong binding activities to CD137 in a dose-responsive manner with EC50 of 2.257 μg/mL (12.90 nM); whereas the negative control human antibodies (hIgG) had no binding to HuT78/CD137 and CT26 OS8-CEA as expected (FIGS. 35 and 37). Similarly, BE-146 showed strong binding activities to CEA in a dose-responsive manner with EC50 of 1.532 μg/mL (8.75 nM); whereas the negative control human antibodies (hIgG) had no binding to HuT78/CD137 and CT26-0S8-CEA as expected (FIGS. 35 and 36).

TABLE 38
Comparative analysis of SPR determined kinetic
parameters to different species of CD137

| Antigen | Antibody | Kₒₙ (1/Ms) | K_{off} (1/s) | K_{D} (M)^{a, b} |
|---|---|---|---|---|
| Human CD137 | BE-146 | 1.30E+05 | 4.69E−03 | 3.62E−08 |
| Monkey CD137 | | 1.67E+05 | 2.66E−03 | 1.59E−08 |
| Mouse CD137 | | ND^{c} | ND | ND |

Abbreviations: K_{D}, affinity constant; K_{off}, rate constant of dissociation; Kₒₙ, rate constant of association; ND, not determinable; SPR, surface plasmon resonance.
K_{D} values are calculated from the ratio of the kinetic constants as K_{D} = K_{off}/Kₒₙ.
K_{D} values are determined by the analyte concentration at which half of the ligands are occupied at equilibrium.
ND: Affinity is too weak for determination.

TABLE 39
Comparative analysis of SPR determined kinetic
parameters to different species of CEA

| Antigen | Antibody | Kₒₙ (1/Ms) | K_{off} (1/s) | K_{D} (M)^{a, b} |
|---|---|---|---|---|
| Human CEA | BE-146 | 9.85E+04 | 2.95E−04 | 3.00E−09 |
| Monkey CEA | | 6.30E+04 | 7.17E−04 | 1.14E−08 |

Abbreviations: CEA, carcinoembryonic antigen; K_{D}, affinity constant; K_{off}, rate constant of dissociation; Kₒₙ, rate constant of association; ND, not determinable; SPR, surface plasmon resonance.
K_{D} values are calculated from the ratio of the kinetic constants as K_{D} = K_{off}/Kₒₙ.
K_{D} values are determined by the analyte concentration at which half of the ligands are occupied at equilibrium.

### Example 31. CEA×CD137 Induces T Cell Activation in a CEA Dependent Manner

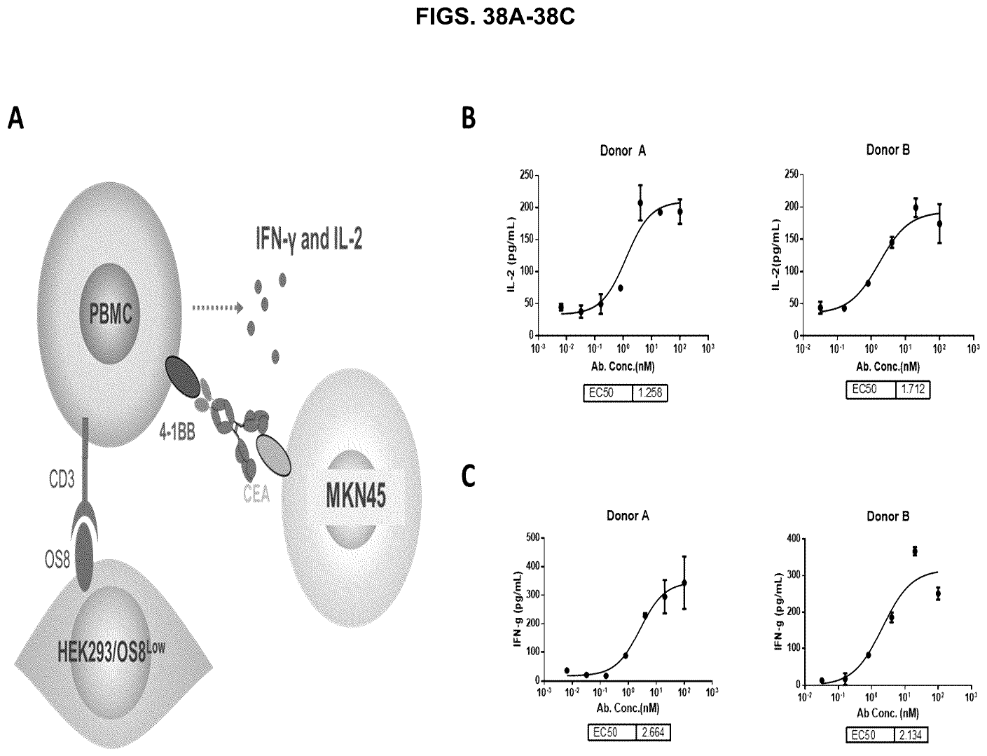

The functionality of CEA×CD137 multispecific antibody BE-146 was assessed in different in vitro experiments. We first used human peripheral blood mononuclear cells (PBMCs) from healthy donors to activate human T cells with CEA×CD137 and HEK293/OS8 providing signal. PBMCs were isolated from whole blood of healthy donors by Ficoll (Histopaque-1077, Sigma-St. Louis MO) separation. To determine whether CEA×CD137 could induce cytokine release from human PBMCs in the presence of CEA⁺ tumor cells, PBMCs (1×10⁵/well) were co-cultured with CEA⁺ MKN45 cells (2×10⁵/well) and HEK293/OS8 (1×10⁵/well) cell for 2 days (FIG. 38A) in 96-well v-bottom plates. IL-2 and IFN-γ release from PBMCs were determined by ELISA. The results showed that CEA×CD137 could induce significant cytokine release (FIGS. 38B-38C). PBMCs from 2 donors were tested. Results were shown in mean±SD of duplicates.

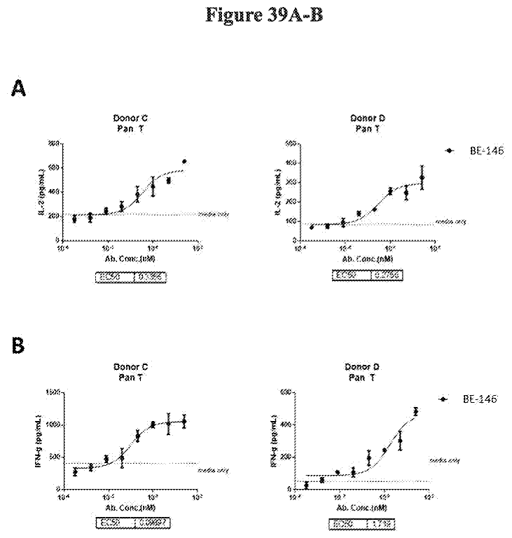

We next investigated whether CEA×CD137 can enhance antigen-specific CD8⁺ T cell function. Human peripheral blood mononuclear cells (PBMCs) were isolated from whole blood of healthy donors by Ficoll (Histopaque-1077, Sigma-St. Louis MO) separation. T cells were isolated using the human Pan T cell isolation kit (Miltenyi, Cat. 130-096-535). To determine whether BE-146 could induce cytokine release from human T cells in the presence of CEA⁺ tumor cells, T cells (1×10⁵/well) were co-cultured with CEA+MKN45 cells (2×10⁵/well) and HEK293/OS8 (1×10⁵/well) cell for 2 days in 96-well v-bottom plates. IL-2 and IFN-γ release from T cells were determined by ELISA. The results showed that multispecific antibody BE-146 could induce significant IL-2 (FIG. 39A) and IFN-γ (FIG. 39B) release.

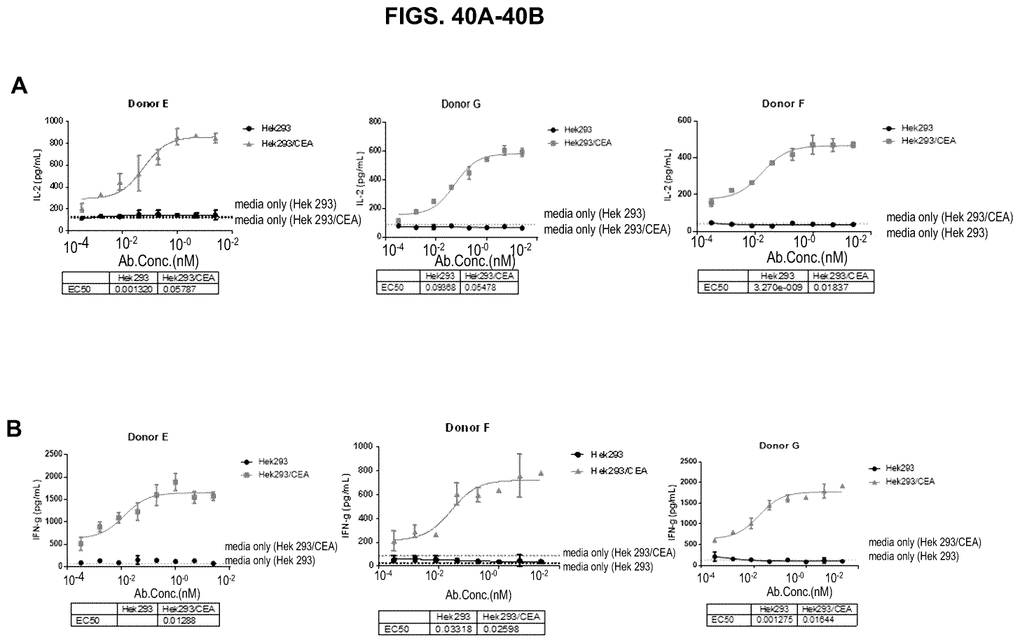

We then investigated whether CEA×CD137 can induce a response that was CEA dependent. Human peripheral blood mononuclear cells (PBMCs) were isolated from whole blood of healthy donors by Ficoll (Histopaque-1077, Sigma-St. Louis MO) separation. To determine whether CEA×CD137 induced cytokine release from human PBMCs was CEA dependent, PBMCs (1×10⁵/well) were co-cultured with HEK293 or CEA over-expressing HEK293 cells (HEK293/CEA) (1×10⁵/well) as target cells, and HEK293/OS8 (1×10⁵/well) cell for 2 days in 96-well v-bottom plates. IL-2 and IFN-γ release from PBMCs were determined by ELISA. The results showed that multispecific antibody BE-146 could induce significant IL-2 and IFN-γ release from PBMCs against CEA over-expressing HEK293 cells, but not against HEK293 cells without CEA transduction (FIGS. 40A-B).

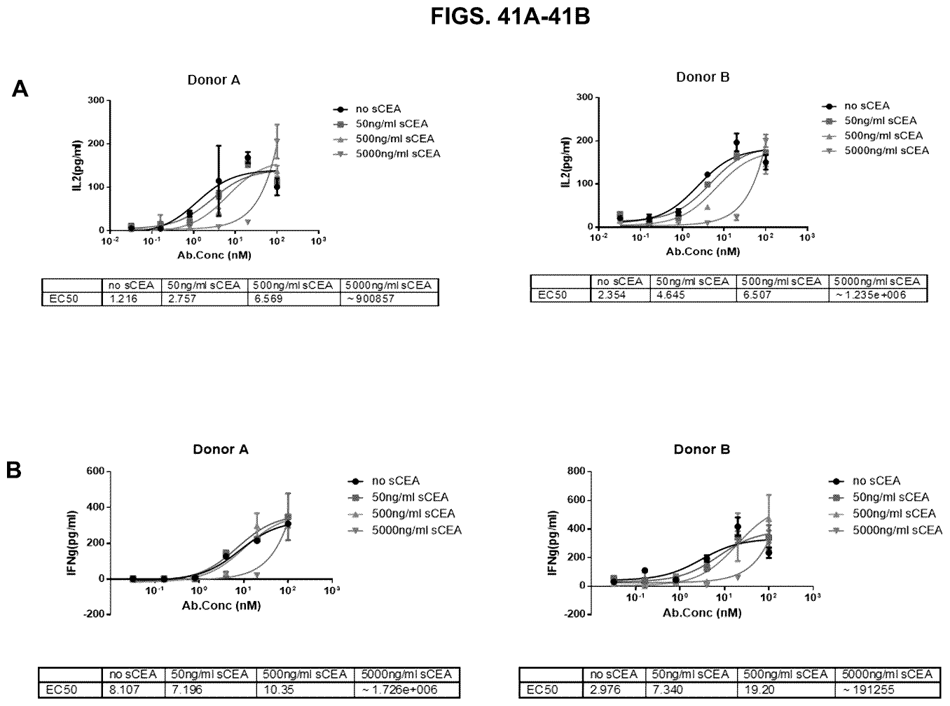

In addition, a series of experiments were conducted to determine whether the induced response from the CEA×CD137 construct could be blocked by soluble CEA. Human peripheral blood mononuclear cells (PBMCs) were isolated from whole blood of healthy donors by Ficoll (Histopaque-1077, Sigma-St. Louis MO) separation. To determine whether BE-146 induced cytokine release from human PBMCs could be blocked by soluble CEA, PBMCs (1×10⁵/well) were co-cultured with MKN45 (1×10⁵/well), and HEK293/OS8 (1×10⁵/well) cell for 2 days in 96-well v-bottom plates, in the presence of different concentrations of recombinant soluble CEA. IL-2 and IFN-γ release from PBMCs were determined by ELISA. The results showed that the multispecific antibody BE-146 induced IL-2 (FIG. 41A) and IFN-γ (FIG. 41B) release from PBMCs and this release was not significantly blocked by 50 ng/ml or 500 ng/ml soluble CEA. Only extremely high concentrations of CEA (5000 ng/ml) led to a reduction.

These data show that CEA×CD137 in the presence of (CD3F or T cell receptor stimulation) induces strong, CEA-dependent T cell activation.

### Example 32. BE-146 Enhances T Cell Activation

A cell-based bioluminescent assay was developed and used to measure the activity of BE-146 which target and stimulate an inducible costimulatory receptor CD137 and enhances T cell activation.

Two genetically modified cell lines, JK-NFKB-CD137 and CT26-OS8-CEA, were used as effector cells and target cells respectively in this assay. JK-NFKB-CD137 was developed from the Jurkat cell line, clone E6-1 (ATCC, TIB-152) by stably transfecting a human CD137 gene vector and a luciferase construct with a NF-kB response element that can respond to both T cell receptor (TCR) activation and CD137 co-stimulation. CT26-OS8-CEA cell line was generated from CT26WT cells by ectopically expressing a human CEA and the T cell engager OS8 (a membrane-bound form of anti-CD3 antibody). When the two cell lines are co-cultured, addition of the bispecific antibody BE-146 would interact with both CD137 expressing on the effector cells and CEA expressing on the target cells and initiate the CEA-dependent CD137 co-stimulation and activation of luciferase gene promoter in a dose dependent manner. JK-NFKB-CD137 (5×10⁴ cells/well) and CT26-0S8-CEA (1×10⁴ cells/well) were co-cultured for 5-6 hours in the presence of serially diluted BE-146. As a negative control, human IgG (hIgG) and a buffer containing no antibody was used.

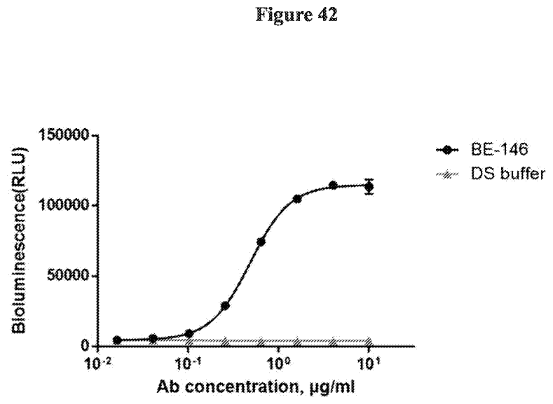

BE-146 showed agonistic functional activity in a dose-responsive manner. This experiment was performed in duplicate and the EC50 for BE-146 was 0.51 μg/mL (2.91 nM) and 0.56 μg/mL (3.20 nM) as shown in FIG. 42. The buffer and human IgG controls had no activity.

### Example 33. BE-146 Enhances IFN-γ and IL-2 Production from Human PBMCs in an CEA Dependent Manner

Hek293/OS8^{low} cell line was generated by retroviral transduction with the T-cell engager OS8 (a membrane-bound form of anti-CD3 antibody) to provide anti-CD3 stimulation for the initial T cell activation. PBMCs from healthy donors and Hek293/OS8^{low} were co-cultured with target cells MKN45, which have CEA high expression or NCI-N87, which express only low amounts of CEA, in the presence of BE-146 or Urelumab, as a reference antibody or human IgG1 as a negative control. PBMCs (3×10⁴ cells/well) were co-cultured for 48 hours with Hek293/OS8^{low} (1×10⁴ cells/well) and MKN45 (2×10⁴ cells/well) in the presence of serially diluted BE-146, Urelumab or huIgG1. PBMCs (3×10⁴ cells/well) were co-cultured for 48 hours with Hek293/OS8^{low} (1×10⁴ cells/well) and NCI-N87 (2×10⁴ cells/well) in the presence of serially diluted BE-146, Urelumab or huIgG1. IFN-γ and IL-2 release were measured by ELISA.

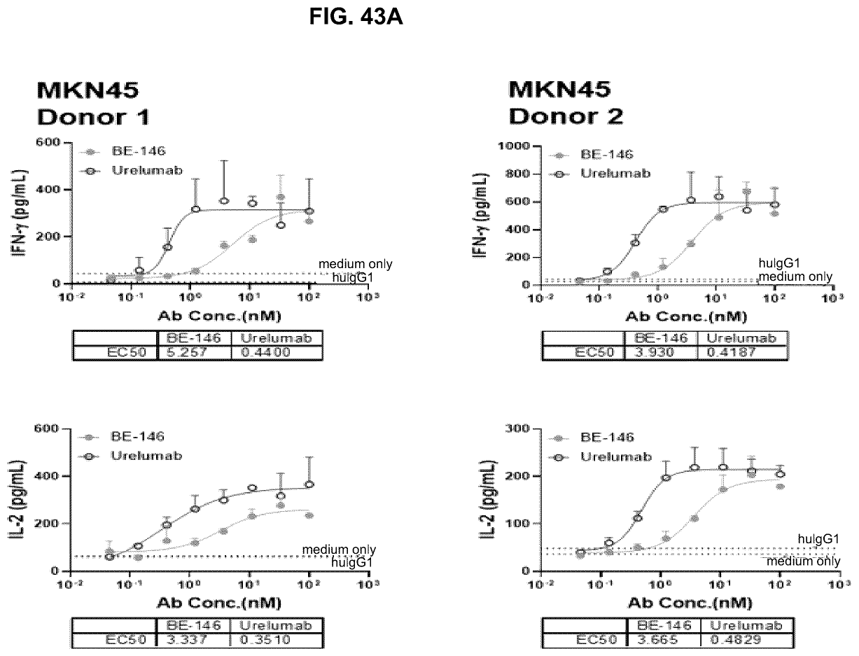

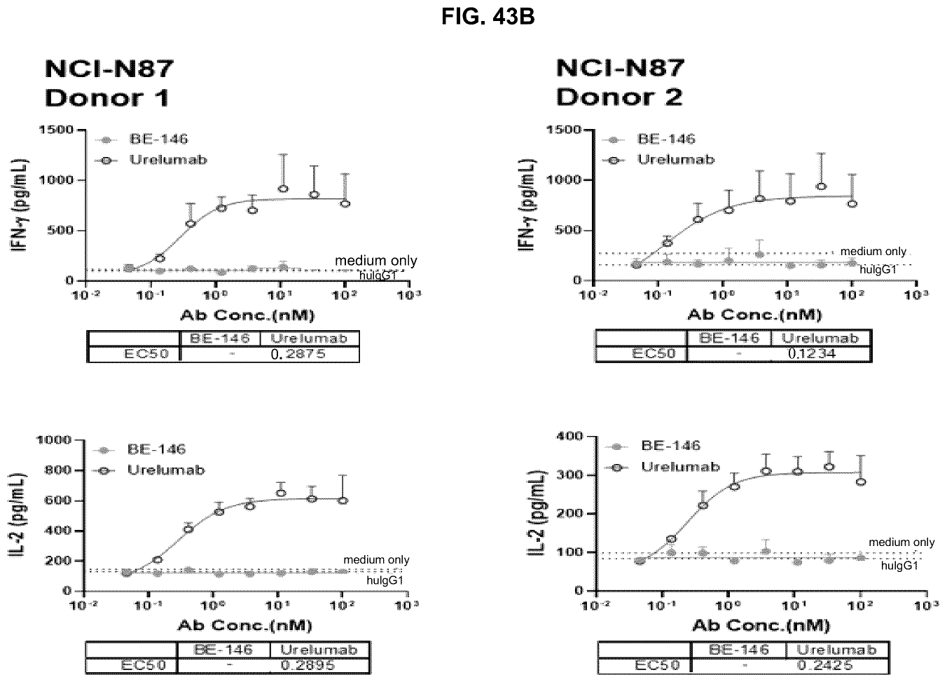

BE-146 promoted PBMCs from both donors to secrete IFN-γ and IL-2 in a dose-dependent manner when the target cells were MKN45 (CEA high) (FIG. 43A). However, when NCI-N87(CEA low) cells were used as target cells, BE-146 induced no cytokine release (FIG. 43B).

### Example 34. BE-146 Enhances Cytotoxicity of Human PBMCs Against MKN45 Cells

PBMCs from healthy donors were co-cultured with MKN45 as the target cells, in the presence of BE-146. Urelumab was used as a reference antibody, with human IgG1 as a negative control. Solitomab, which is an EpCam/CD3 bispecific T-cell engager (BiTE) construct (Ferrari et al., J Exp Clin Cancer Res 2015; 34:123), was added into the co-culture system at 10 pg/mL concentration to provide anti-CD3 stimulation for the initial T cell activation. MKN45(1×10⁴ cells/well) cells were pre-cultured for 24 hours to allow the cells to adhere to the plate, then co-cultured with PBMCs (1×10⁵ cells/well) in the presence of BE-146, or Urelumab. Solitomab (10 pg/mL) was added into the co-culture system to offer the initial stimulation.

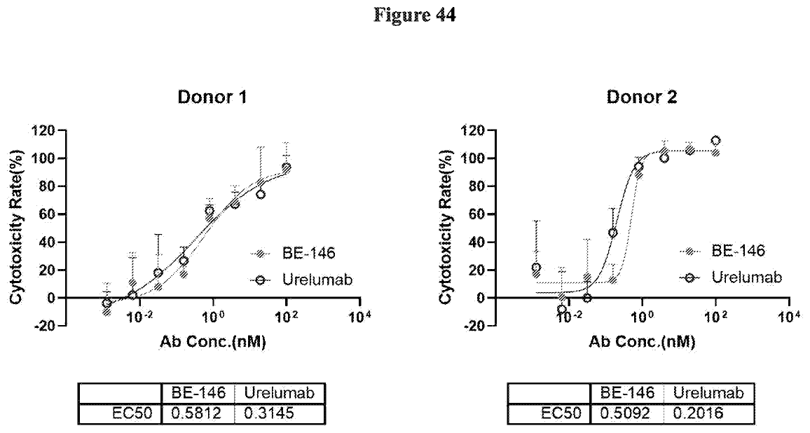

The killing towards MKN45 cells was measured via monitoring alterations in MKN45 adhesion to the underlying extracellular matrix, using the real-time cell analysis (RTCA) system (Hamidi, Lilja and Ivaska Bio Protoc 2017; 7(24):e2646). BE-146 induced a dose-dependent killing towards MKN45 tumor cells. The average EC50 of BE-146 from 2 donors is 0.5452 nM at a comparable level to Urelumab (EC50=0.258 nM) (FIG. 44).

### Example 35. CEA×CD137 Antibodies in Combination with Anti-PD-1 Antibody Tislelizumab Further Promotes Immune Cell Activation

Costimulatory receptors CD137 can induce T-cell activation intracellular signals, but the signals can be repressed by immune-checkpoint ligation, such as PD-1/PD-L1. Therefore, the combination of PD-1 antibody Tislelizumab (BGB-A317) with BE-146 can have increased efficacy. To determine whether BE-146 in combination with the anti-PD-1 antibody Tislelizumab could enhance immune cell activation compared to monotherapy, human PBMCs were co-cultured with CEA and PD-L1 expressing target cells, and the IFN-γ release was determined as functional readout. PBMCs were used as effector cells. Hek293/OS8-PDL1 cells, which were engineered to express PD-L1 and a T-cell engager OS8, were mixed with MKN45 (CEA high) as target cells. IFN-γ secretion was determined as a marker for T cell activation.

PBMCs were pre-stimulated with 40 ng/mL OKT3 for 2 days. The stimulated PBMCs (3×10⁴ cells/well) were co cultured with Hek293/OS8-PDL1(1×10⁴ cells/well) and MKN45 (2×10⁴ cells/well) for 48 hours in the presence of serially diluted BE-146 and Tislelizumab (1000 ng/mL). IFN-γ release was measured by ELISA as readout.

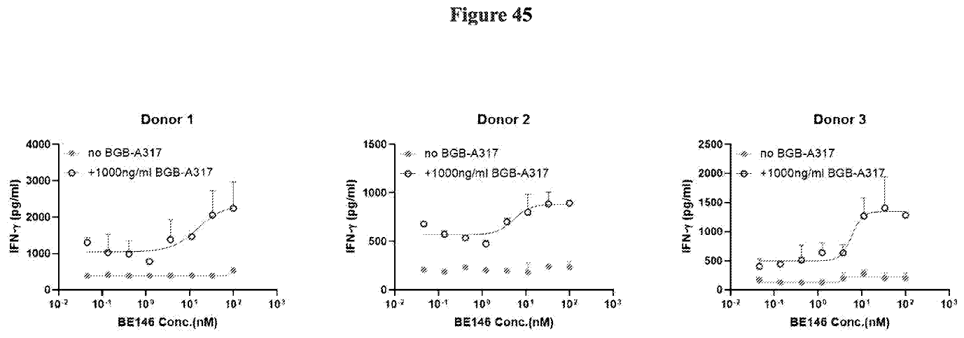

The combined administration of BE-146 and Tislelizumab demonstrated a cumulative effect on human T-cell activation, when PBMCs are cocultured with Hek293/OS8-PD-L1 cells and MKN45 (CEA high) cells. The combination of BE-146 and Tislelizumab significantly enhanced IFN-γ production relative to BE-146 or Tislelizumab alone as shown in FIG. 45.

Tislelizumab (BGB-A317) is disclosed in U.S. Pat. No. 8,735,553 and the VH/VL sequences are shown in Table 40 below.

TABLE 40
Tislelizumab sequence table

| | |
|---|---|
| Tislelizumab | QVQLQESGPGLVKPSETLSLTCTVSGFSLTSYG |
| VH | VHWIRQPPGKGLEWIGVIYADGSTNYNPSLKSR |
| | VTISKDTSKNQVSLKLSSVTAADTAVYYCARAY |
| | GNYWYIDVWGQGTTVTVSS (SEQ ID NO: |
| | 315) |
| Tislelizumab | DIVMTQSPDSLAVSLGERATINCKSSESVSNDV |
| VL | AWYQQKPGQPPKLLINYAFHRFTGVPDRFSGSG |
| | YGTDFTLTISSLQAEDVAVYYCHQAYSSPYTFG |
| | QGTKLEIK (SEQ ID NO: 316) |

### Example 36. Effector Receptor Binding and Effector Functions

BE-146 uses an engineered human IgG1 Fc moiety, which has diminished binding activities to effector function receptors. ELISA assays demonstrated that BE-146 has reduced binding activities to FcγRI, FcγRIIAH131, FcγRIIAR131, FcγRIIB, FcγRIIIAV158, FcγRIIIAF158, FcγRIIIB and C1q when BE-146 was compared to human IgGs (huIgG). As FcγRs and C1q are the key receptors mediating immune complex-induced effector functions, BE-146 has undetectable effector functions, such as antibody dependent cellular cytotoxicity (ADCC), antibody dependent cellular phagocytosis (ADCP) and complement-dependent cytotoxicity (CDC).

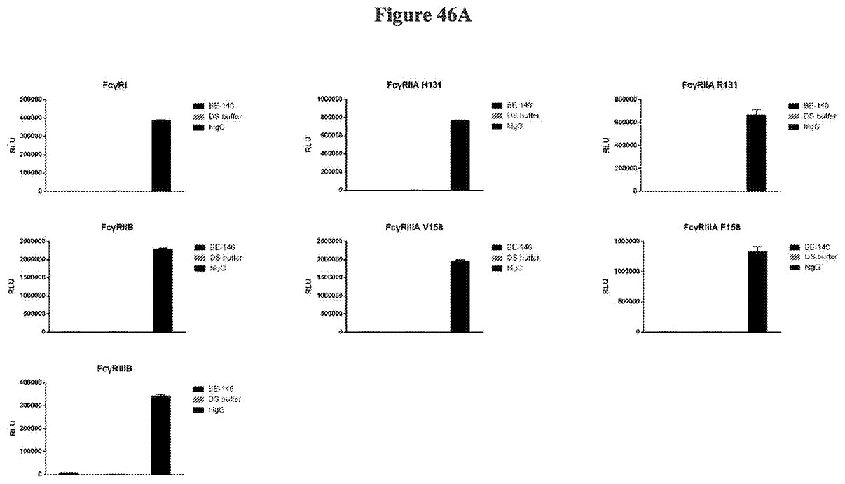

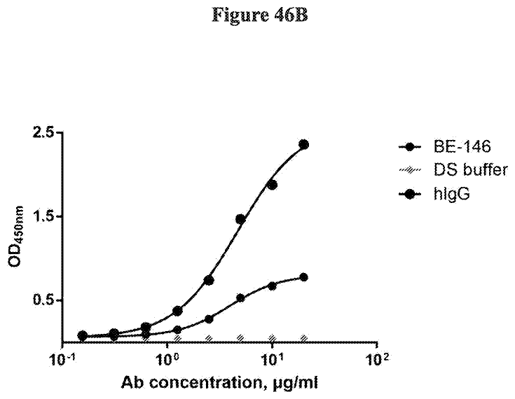

FcγR binding activities were assessed by ELISA. BE-146 did not exhibit any significant binding activity to FcγRI, FcγRIIAH131, FcγRIIAR131, FcγRIIB, FcγRIIIAV158, FcγRIIIAF158, FcγRIIIB, which were comparable to negative control. In contrast, the positive control human IgG produced a strong binding signal to any of the FcγRs in the assay (FIG. 46A-B).

### Example 37. CEA×CD137 Antibodies Reduce Tumors In Vivo

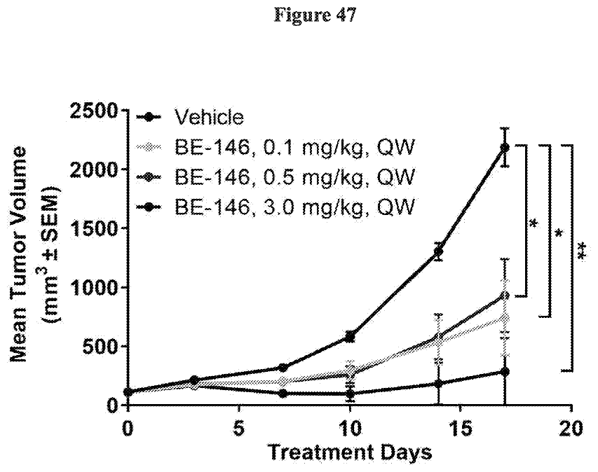

The in vivo efficacy of BE-146 was examined in the MC38/hCEA mouse colon adenocarcinoma model in humanized CD137 knock-in mice. MC38/hCEA cells were implanted into female humanized CD137 knock-in mice. On Day 5 after cell inoculation, the mice were randomized into 4 groups according to tumor volume. Intraperitoneal administration of BE-146 (0.1, 0.5, and 3.0 mg/kg, once weekly) effectively inhibited tumor growth, the TGI rates on Day 17 were 70%, 61% and 92%, respectively (FIG. 47). In addition, the ratio of tumor free in 0.1, 0.5 and 3.0 mg/kg group was 20%, 30%, and 90% at study endpoint (Day 21), respectively. The percentage of tumor free animals was increased over dosage from 0.1 to 3.0 mg/kg. Pharmacokinetic (PK) profiles of BE-146 at the 3 dosage levels (0.1, 0.5, and 3.0 mg/kg) were characterized after the first dosing. Drug exposure of BE-146 (AUC₀₋₁₆₈ₕ and Cₘₐₓ) were increased proportionally (Table 41). There was no significant impact on animal body weight in any of the treatment group throughout the study.

TABLE 41
Efficacy and PK Parameters of BE-146 in the MC38/hCEA
syngeneic tumor model in humanized CD137 knock-in mice

| | | | | Mean | | PK parameter | |
|---|---|---|---|---|---|---|---|
| | | | | tumor | | (single does) | |
| | | | | volume | p value | Cₘₐₓ | AUC_{0-168 h} |
| | | | TGI | (mm³ ± | vs. | (μg/mL) | (h · μg/mL) |
| | | Dose | (%) | SEM) | vehicle | (mean ± | (mean ± |
| Antibody | n | (mg/kg) | (Day 17) | (Day 17) | (Day 17) | SEM) | SEM) |
| Vehicle | 10 | NA | NA | 2187.0 | NA | NA | NA |
| | | | | (±161.4) | | | |
| BE-146 | 10 | 0.1, QW | 70 | 741.7 | 0.0143 | 0.60 | 42.34 |
| | | | | (±316.7) | | (±0.04) | (±1.92) |
| BE-146 | 10 | 0.5, QW | 61 | 930.9 | 0.0077 | 3.04 | 304.66 |
| | | | | (±309.3) | | (±0.35) | (±29.75) |
| BE-146 | 10 | 3.0, QW | 92 | 286.3 | 0.0014 | 26.11 | 2278.75 |
| | | | | (±284.7) | | (±1.39) | (±334.34) |

Abbreviations: AUC_{0-168 h}, area under the serum concentration-time curve from 0 to 168 hours; Cₘₐₓ, maximum concentration; hCEA, carcinoembryonic antigen; n, number of animals; NA, not applicable; PK, pharmacokinetics; QW, once weekly; SEM, standard error of the mean; TGI, tumor growth inhibition.
Note:
TGI rate was calculated according to the following formula: % TGI = [1 − (treated Tt − treated T0)/(vehicle Tt − vehicle T0)] × 100%. This table shows the TGI on Day 17. Treated Tt = mean tumor volume of a dosing group on Day t; treated T0 = mean tumor volume of a dosing group on Day 0; vehicle Tt = mean tumor volume of vehicle group on Day t; and vehicle T0 = mean tumor volume of vehicle group on Day 0.

### Example 38. Combination Treatment of Anti-PD-1 Antibody and CEA×CD137 Increases Tumor Regression

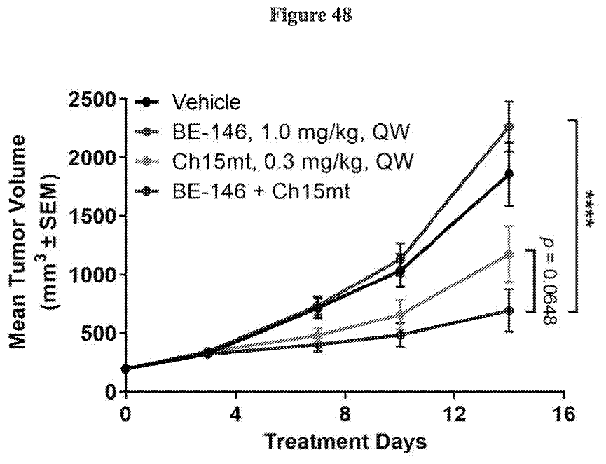

The antitumor activity of the combination of BE-146 and anti-mouse PD-1 antibody was investigated in the CT26/hCEA syngeneic model in humanized CD137 knock-in mice. CT26/hCEA cells were implanted into female humanized CD137 knock-in mice. On Day 7 after cell inoculation, the mice were randomized into 4 groups according to tumor volume. Mice receiving the combination treatment of BE-146 (1.0 mg/kg, once weekly) and anti-mouse PD-1 antibody Ch15mt (0.3 mg/kg, once weekly) exhibited synergistic tumor growth inhibition. The tumor growth inhibition rate on Day 14 was 70%, which was significantly higher than that in the group treated with BE-146 (−24%) or Ch15mt (41%) alone. As compared to vehicle control or single-agent treatments, the combination of BE-146 and anti-PD-1 induced significantly increased anti-tumor effects, summarized in Table 42 and shown in FIG. 48.

TABLE 42
Antitumor Effect of BE-146 and Ch15mt in the CT26/hCEA
syngeneic model in humanized CD137 Knock-in Mice

| | | | | | P value |
|---|---|---|---|---|---|
| | | | | Mean tumor | vs. BE-146 + |
| | | Dose | TGI (%) | volume (mm³ ± | Ch15mt group |
| Treatment | n | (mg/kg) | (Day 14) | SEM) (Day 14) | (Day 14) |
| Vehicle | 10 | NA | NA | 1856.8 ± 273.6 | NA |
| BE-146 | 10 | 1.0 QW | −24 | 2264.5 ± 213.4 | 0.0000 |
| Ch15mt | 10 | 0.3 QW | 41 | 1173.0 ± 238.3 | 0.0648 |
| BE-146 + | 10 | 1.0 + | 70 | 692.1 ± 180.5 | NA |
| Ch15mt | | 0.3 QW | | | |

Abbreviations: hCEA, human carcinoembryonic antigen; n, number of animals; NA, not applicable; QW, weekly; SEM, standard error of the mean; TGI, tumor growth inhibition.

### Example 39. Efficacy of the Combination of BE-146 and Anti-PD-1 Antibody in B16-F10/hCEA Model in Humanized CD137 Knock-In Mice

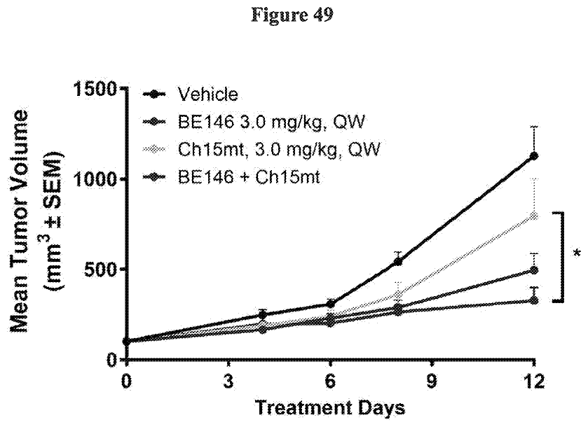

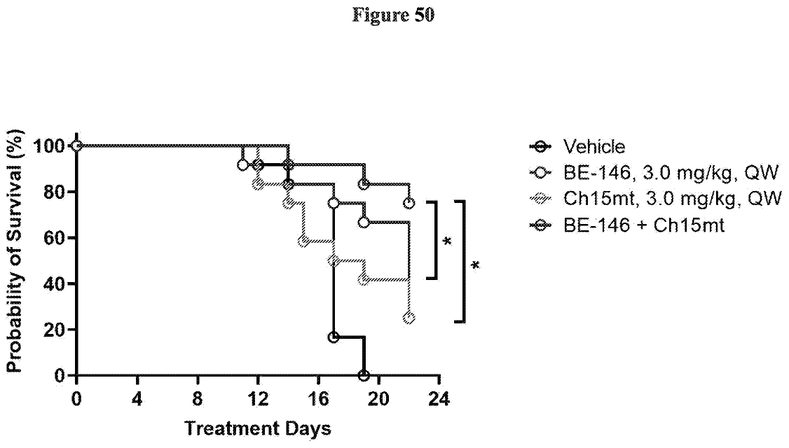

The antitumor activity of the combination of BE-146 and anti-mouse PD-1 antibody was investigated in the B16-F10/hCEA syngeneic model in humanized CD137 knock-in mice. B16-F10 is murine melanoma cell line. Mice receiving the combination treatment of BE-146 (3.0 mg/kg, once weekly) and anti-mouse PD-1 antibody Ch15mt (3.0 mg/kg, once weekly) had significant tumor growth inhibition (TGI). On Day 12 of the combination antibody treatment, the TGI was 78% as shown in FIG. 49 and Table 43. In addition, combination treatment of BE-146 and Ch15mt significantly improved the survival rate of animals. The survival rate at study endpoint was 75%, which was higher than that in the monotherapy group with BE-146 (25%) or Ch15mt alone (25%) (FIG. 50 and Table 43). There was no significant impact on animal body weight in any treatment group throughout the study.

TABLE 43
Anti-tumor effect of BE-146 and Ch15mt in a B16-F10/hCEA
syngeneic model in humanized CD137 knock-in mice

| | | | | Mean | | | P value vs. |
|---|---|---|---|---|---|---|---|
| | | | | tumor | | | combination |
| | | | | volume | P value vs. | Survival | group |
| | | | TGI | (mm³ ± | combination | rate | On survival |
| | | Dose | (%) | SEM) | group | (%) | rate |
| Treatment | n | (mg/kg) | (Day 12) | (Day 12) | (Day 12) | (Day 22) | (Day 22) |
| Vehicle | 12 | NA | NA | 1126.9 ± | NA | 0 | NA |
| | | | | 161.3 | | | |
| BE-146 | 12 | 3.0 QW | 62 | 495.4 ± | 0.1302 | 25 | 0.02 |
| | | | | 92.7 | | | |
| Ch15mt | 12 | 3.0 QW | 32 | 795.2 ± | 0.0181 | 25 | 0.02 |
| | | | | 205.6 | | | |
| BE-146 + | 12 | 3.0 + | 78 | 328.6 ± | NA | 75 | NA |
| Ch15mt | | 3.0 QW | | 71.3 | | | |

Abbreviations: n, number of animals; NA, not applicable; QW, once weekly; SEM, standard error of the mean; TGI, tumor growth inhibition.
Note:
TGI rate was calculated according to the following formula: % TGI = [1 − (treated Tt − treated T0)/(vehicle Tt − vehicle T0)] × 100%. This table shows the TGI on Day 12. Treated Tt = mean tumor volume of a dosing group on Day t; treated T0 = mean tumor volume of a dosing group on Day 0; vehicle Tt = mean tumor volume of vehicle group on Day t; and vehicle T0 = mean tumor volume of vehicle group on Day 0.

### Example 40. BE-146 Dosing

A flat dose of BE-146 alone or in combination with Tislelizumab (BGB-A317) will be administered via intravenous infusion on Day 1, Day 8, and Day 15 of each 21-day cycle. The planned dose levels of BE-146 to be tested as a monotherapy are 5 mg, 15 mg, 50 mg, 150 mg, 300 mg, 600 mg, and 1200 mg. The dose levels of BE-146 to be tested in combination with 200 mg of tislelizumab are 50 mg, 150 mg, 300 mg, 600 mg, and 1200 mg as shown in Table 44. The dose level and schedule of tislelizumab (i.e., 200 mg administered via intravenous infusion on Day 1 of each 21-day cycle) will remain fixed. When given in combination, tislelizumab will be administered first, followed by BE-146. However, lower, intermediate, and/or higher dose levels and/or alternative dosing intervals of BE-146 in the monotherapy cohort and/or in the cohort receiving a combination of BE-146 and tislelizumab can be determined by the physician.

TABLE 44
BE-146 dosing

| BE-146 Monotherapy | | BE-146 + Tislelizumab |
|---|---|---|
| 5 | mg (QW) | NA |
| 15 | mg (QW) | NA |
| 50 | mg (QW) | 50 mg (QW) + 200 mg tislelizumab (Q3W) |
| 150 | mg (QW) | 150 mg (QW) + 200 mg tislelizumab (Q3W) |
| 300 | mg (QW) | 300 mg (QW) + 200 mg tislelizumab (Q3W) |
| 600 | mg (QW) | 600 mg (QW) + 200 mg tislelizumab (Q3W) |
| 1200 | mg (QW) | 1200 mg (QW) + 200 mg tislelizumab (Q3W) |

Abbreviations: NA, not applicable; QW, weekly; Q3W, once every 3 weeks.

### Example 44. BE-146 Indications

CEA overexpression was observed in many types of cancers, including colorectal cancer (CRC), gastric cancer (GC), lung cancer, pancreatic cancer, hepatocellular carcinoma, breast cancer, and thyroid cancer (Chevinsky A H., Semin Surg Oncol. 1991; 7(3):162-6; Shively et al., Crit Rev Oncol Hematol. 1985; 2(4):355-99). High serum CEA is associated with poor prognosis of patients with GC and lung cancers. (Hall et al., Ann Coloproctol. 2019; 35(6):294-305; Moriyama et al., Surg Today. 2021 October; 51(10):1638-48; Grunnet et al., Lung Cancer. 2012 May; 76(2):138-43). Five year survival rates of the tumors are still low in those with metastatic advanced disease: 14.7% for CRC, 5.5% for GC, and 6.3% for lung cancer.

The overexpression of CEA contributes to immune dysfunctions. CEA was reported to regulate the responses of various types of immune cells. For example, CEA can interact with CEACAM1, which acts as a coinhibitory molecule to reduce natural killer (NK) cell-mediated cytotoxicity (Stern et al., J. Immuno. 2005; 174(11):6692-701). Kupffer cells, upon CEA activation, may induce cytokines such as IL-10, IL-6, and TNF-α (Gangopadhyay et al., Cancer Letters 1997; 118(1):1-6). It was also reported that the activation of human PBMC-derived T cells can by inhibited by CEA (Lee et al., Int. J. Cancer 2015; 136(11):2579-87).

Based on CEA overexpression, treatment with BE-146 will be administered to patients with histologically or cytologically confirmed advanced, metastatic, unresectable CRC, GC or NSCLC. Cohorts of approximately 7 increasing dose levels of BE-146 monotherapy and 5 increasing dose levels of BE-146 in combination with 200 mg of tislelizumab will be sequentially evaluated to evaluate the safety, tolerability, PK, and pharmacodynamics of BE-146 as monotherapy and in combination with tislelizumab, and to determine the efficacy of BE-146 in patients with advanced colorectal cancer (CRC), gastric cancer (GC) or non-small cell lung cancer (NSCLC).

### Example 35. CEA×CD137 Toxicity in Vivo

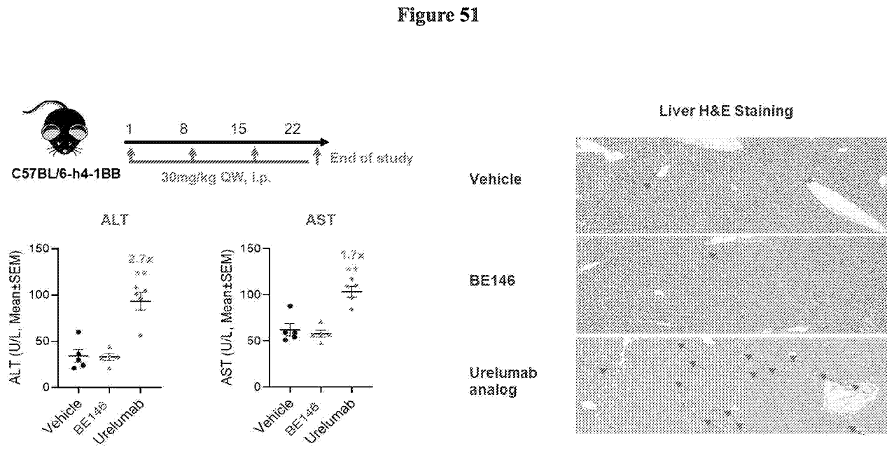

BE-146 or the Urelumab analog antibody (30 mg/kg) were injected into humanized CD137 mice of the C57BL/6 background, once per week for three doses. Blood was collected on day 22 and analyzed by blood biochemical tests. Compared with the vehicle control, high-dose of the Urelumab analog, but not BE-146, induced significantly increased alanine transaminase (ALT) and aspartate aminotransferase (AST) concentrations indicative of liver toxicity. In addition, microscopic changes of increased inflammatory cells were observed in hepatic tissues from the Urelumab analog-treated group while no significant microscopic changes were observed in the BE-146 treated group (FIG. 51). Therefore, BE-146 is a promising combination partner for cancer immunotherapies without liver toxicity, including checkpoint inhibitors and T-cell engagers.

The safety profile of BE-146 was characterized in a 4-week repeated-dose toxicity study in cynomolgus monkeys using a tissue cross-reactivity assay with normal human tissues and a cytokine release assay using fresh human PBMCs. The studies were conducted in accordance with Good Laboratory Practice regulations/principles.

In the repeated-dose study in cynomolgus monkeys treated with BE-146 at doses of 5, 20, or 100 mg/kg once weekly for 4 weeks (5 doses in total) via intravenous infusion, no mortality or adverse effect was noted. The systemic exposure increased dose-proportionally following the first dose. No apparent sex difference or drug accumulation was noted. The no-observed-adverse-effect level (NOAEL) was 100 mg/kg. The NOAEL was considered to be 100 mg/kg, where the steady-state Cmax and AUC0-168 h were 2220 μg/mL and 58,600 h·μg/mL, respectively, in males, and 2110 μg/mL and 66,800 h·μg/mL, respectively, in females. In addition, in the PBMC-based cytokine release assay, compared with human IgG, immobilized BE-146 did not induce a significant release of cytokine/chemokines in any of the tested donor samples, indicating minimal risk of inducing cytokine release syndrome.

### REFERENCES

Abdul-Wahid, A., M. Cydzik, N. W. Fischer, A. Prodeus, J. E. Shively, A. Martel, S. Alminawi, Z. Ghorab, N. L. Berinstein & J. Gariépy (2018) Serum-derived carcinoembryonic antigen (CEA) activates fibroblasts to induce a local re-modeling of the extracellular matrix that favors the engraftment of CEA-expressing tumor cells. International Journal of Cancer, 143, 1963-1977.
Abdul-Wahid, A., E. H. B. Huang, M. Cydzik, E. Bolewska-Pedyczak & J. Gariepy (2014) The carcinoembryonic antigen IgV-like N domain plays a critical role in the implantation of metastatic tumor cells. Molecular Oncology, 8, 337-350.
Arnon, T. I., H. Achdout, O. Levi, G. Markel, N. Saleh, G. Katz, R. Gazit, T. Gonen-Gross, J. Hanna, E. Nahari, A. Porgador, A. Honigman, B. Plachter, D. Mevorach, D. G. Wolf & O. Mandelboim (2005) Inhibition of the CD137 activating receptor by pp65 of human cytomegalovirus. Nat Immunol, 6, 515-23.
Augugliaro, R., S. Parolini, R. Castriconi, E. Marcenaro, C. Cantoni, M. Nanni, L. Moretta, A. Moretta & C. Bottino (2003) Selective cross-talk among natural cytotoxicity receptors in human natural killer cells. Eur J Immunol, 33, 1235-41.
Bacac, M., C. Klein & P. Umana (2016) CEA TCB: A novel head-to-tail 2:1 T cell bispecific antibody for treatment of CEA-positive solid tumors. Oncoimmunology, 5, e1203498.
Barrow, A. D., C. J. Martin & M. Colonna (2019) The Natural Cytotoxicity Receptors in Health and Disease. Front Immunol, 10, 909.
Beauchemin, N. 2017. CEA Gene Family. In Encyclopedia of Cancer, ed. M. Schwab, 870-874. Berlin, Heidelberg: Springer Berlin Heidelberg.
Beck, A., T. Wurch, C. Bailly & N. Corvaia (2010) Strategies and challenges for the next generation of therapeutic antibodies. Nat Rev Immunol, 10, 345-52.
Biron, C. A., K. B. Nguyen, G. C. Pien, L. P. Cousens & T. P. Salazar-Mather (1999) Natural killer cells in antiviral defense: function and regulation by innate cytokines. Annu Rev Immunol, 17, 189-220.
Bozzano, F., A. Picciotto, P. Costa, F. Marras, V. Fazio, I. Hirsch, D. Olive, L. Moretta & A. De Maria (2011) Activating NK cell receptor expression/function (CD137, NKp46, DNAM-1) during chronic viraemic HCV infection is associated with the outcome of combined treatment. Eur J Immunol, 41, 2905-14.
Brinkmann, U. & R. E. Kontermann (2017) The making of bispecific antibodies. MAbs, 9, 182-212.
Brocks, B., P. Garin-Chesa, E. Behrle, J. E. Park, W. J. Rettig, K. Pfizenmaier & D. Moosmayer (2001) Species-crossreactive scFv against the tumor stroma marker “fibroblast activation protein” selected by phage display from an immunized FAP−/− knock-out mouse. Molecular medicine (Cambridge, Mass.), 7, 461-469.
Castriconi, R., C. Cantoni, M. Della Chiesa, M. Vitale, E. Marcenaro, R. Conte, R. Biassoni, C. Bottino, L. Moretta & A. Moretta (2003) Transforming growth factor beta 1 inhibits expression of CD137 and NKG2D receptors: consequences for the NK-mediated killing of dendritic cells. Proc Natl Acad Sci USA, 100, 4120-5.
Chau, I., M. J. Allen, D. Cunningham, A. R. Norman, G. Brown, H. E. Ford, N. Tebbutt, D. Tait, M. Hill, P. J. Ross & J. Oates (2004) The value of routine serum carcino-embryonic antigen measurement and computed tomography in the surveillance of patients after adjuvant chemotherapy for colorectal cancer. J Clin Oncol, 22, 1420-9.
Correia, M. P., A. Stojanovic, K. Bauer, D. Juraeva, L. O. Tykocinski, H. M. Lorenz, B. Brors & A. Cerwenka (2018) Distinct human circulating CD137(+)FcepsilonRIgamma(+)CD8(+) T cell population exhibiting high natural killer-like antitumor potential. Proc Natl Acad Sci US A, 115, E5980-E5989.
Dahlen, E., N. Veitonmaki & P. Norlen (2018) Bispecific antibodies in cancer immunotherapy. Ther Adv Vaccines Immunother, 6, 3-17.
Delahaye, N. F., S. Rusakiewicz, I. Martins, C. Menard, S. Roux, L. Lyonnet, P. Paul, M. Sarabi, N. Chaput, M. Semeraro, V. Minard-Colin, V. Poirier-Colame, K. Chaba, C. Flament, V. Baud, H. Authier, S. Kerdine-Romer, M. Pallardy, I. Cremer, L. Peaudecerf, B. Rocha, D. Valteau-Couanet, J. C. Gutierrez, J. A. Nunes, F. Commo, S. Bonvalot, N. Ibrahim, P. Terrier, P. Opolon, C. Bottino, A. Moretta, J. Tavernier, P. Rihet, J. M. Coindre, J. Y. Blay, N. Isambert, J. F. Emile, E. Vivier, A. Lecesne, G. Kroemer & L. Zitvogel (2011) Alternatively spliced CD137 isoforms affect the prognosis of gastrointestinal stromal tumors. Nat Med, 17, 700-7.
Dotan, E., A. Starodub, J. Berlin, C. H. Lieu, M. J. Guarino, J. Marshall, J. R. Hecht, S. J. Cohen, W. A. Messersmith, P. P. Maliakal, W. A. Wegener, R. M. Sharkey & D. M. Goldenberg (2015) A new anti-CEA-SN-38 antibody-drug conjugate (ADC), IMMU-130, is active in controlling metastatic colorectal cancer (mCRC) in patients (pts) refractory or relapsing after irinotecan-containing chemotherapies: Initial results of a phase I/II study. Journal of Clinical Oncology, 33, 2505-2505.
Durbin, H., S. Young, L. M. Stewart, F. Wrba, A. J. Rowan, D. Snary & W. F. Bodmer (1994) An epitope on carcinoembryonic antigen defined by the clinically relevant antibody PR1A3. Proc Natl Acad Sci USA, 91, 4313-7.
Engler, F. A., J. R. Polli, T. Li, B. An, M. Otteneder, J. Qu & J. P. Balthasar (2018) “Catch-and-Release” Anti-Carcinoembryonic Antigen Monoclonal Antibody Leads to Greater Plasma and Tumor Exposure in a Mouse Model of Colorectal Cancer. J Pharmacol Exp Ther, 366, 205-219.
Fischer, E., K. Chaitanya, T. Wuest, A. Wadle, A. M. Scott, M. van den Broek, R. Schibli, S. Bauer & C. Renner (2012) Radioimmunotherapy of fibroblast activation protein positive tumors by rapidly internalizing antibodies. Clin Cancer Res, 18, 6208-18.
Flamini, E., L. Mercatali, O. Nanni, D. Calistri, R. Nunziatini, W. Zoli, P. Rosetti, N. Gardini, A. Lattuneddu, G. M. Verdecchia & D. Amadori (2006) Free DNA and carcinoembryonic antigen serum levels: an important combination for diagnosis of colorectal cancer. Clin Cancer Res, 12, 6985-8.
Gangopadhyay, A., D. A. Lazure & P. Thomas (1997) Carcinoembryonic antigen induces signal transduction in Kupffer cells. Cancer Letters, 118, 1-6.
Gauthier, L., A. Morel, N. Anceriz, B. Rossi, A. Blanchard-Alvarez, G. Grondin, S. Trichard, C. Cesari, M. Sapet, F. Bosco, H. Rispaud-Blanc, F. Guillot, S. Cornen, A. Roussel, B. Amigues, G. Habif, F. Caraguel, S. Arrufat, R. Remark, F. Romagne, Y. Morel, E. Narni-Mancinelli & E. Vivier (2019) Multifunctional Natural Killer Cell Engagers Targeting NKp46 Trigger Protective Tumor Immunity. Cell, 177, 1701-1713 e16.
Gold, P. & S. O. Freedman (1965) Demonstration of tumor-specific antigens in human colonic carcinomata by immunological tolerance and absorption techniques. J Exp Med, 121, 439-62.
Goldenberg, D. M. (1992) Cancer imaging with CEA antibodies: historical and current perspectives. Int J Biol Markers, 7, 183-8.
Hammarstrom, S. (1999) The carcinoembryonic antigen (CEA) family: structures, suggested functions and expression in normal and malignant tissues. Seminars in Cancer Biology, 9, 67-81.
Hezareh, M., A. J. Hessell, R. C. Jensen, J. G. van de Winkel & P. W. Parren (2001) Effector function activities of a panel of mutants of a broadly neutralizing antibody against human immunodeficiency virus type 1. J Virol, 75, 12161-8.
Hohenberger, P., P. M. Schlag, T. Gerneth & C. Herfarth (1994) Pre- and postoperative carcinoembryonic antigen determinations in hepatic resection for colorectal metastases. Predictive value and implications for adjuvant treatment based on multivariate analysis. Ann Surg, 219, 135-43.
Hollyoake, M., R. D. Campbell & B. Aguado (2005) CD137 (NCR3) is a pseudogene in 12 inbred and wild mouse strains, but an expressed gene in Mus caroli. Mol Biol Evol, 22, 1661-72.

Hsieh, C. L., K. Nagasaki, O. M. Martinez & S. M. Krams (2006) CD137 is a functional activation receptor on a subset of rat natural killer cells. Eur J Immunol, 36, 2170-80.
Hudspeth, K., M. Fogli, D. V. Correia, J. Mikulak, A. Roberto, S. Della Bella, B. Silva-Santos & D. Mavilio (2012) Engagement of CD137 on Vdeltal T cells induces the production of CCL3, CCL4, and CCL5 and suppresses HIV-1 replication. Blood, 119, 4013-6.
Hudspeth, K., B. Silva-Santos & D. Mavilio (2013) Natural cytotoxicity receptors: broader expression patterns and functions in innate and adaptive immune cells. Front Immunol, 4, 69.
Hurwitz, E., I. Stancovski, M. Sela & Y. Yarden (1995) Suppression and promotion of tumor growth by monoclonal antibodies to ErbB-2 differentially correlate with cellular uptake. Proc Natl Acad Sci USA, 92, 3353-7.
Igawa, T., H. Tsunoda, Y. Kikuchi, M. Yoshida, M. Tanaka, A. Koga, Y. Sekimori, T. Orita, Y. Aso, K. Hattori & M. Tsuchiya (2010) VH/VL interface engineering to promote selective expression and inhibit conformational isomerization of thrombopoietin receptor agonist single-chain diabody. Protein Eng Des Sel, 23, 667-77.
Joyce, M. G., P. Tran, M. A. Zhuravleva, J. Jaw, M. Colonna & P. D. Sun (2011) Crystal structure of human natural cytotoxicity receptor CD137 and identification of its ligand binding site. Proc Natl Acad Sci USA, 108, 6223-8.
Koch, J., A. Steinle, C. Watzl & O. Mandelboim (2013) Activating natural cytotoxicity receptors of natural killer cells in cancer and infection. Trends Immunol, 34, 182-91.
Kuespert, K., S. Pils & C. R. Hauck (2006) CEACAMs: their role in physiology and pathophysiology. Current Opinion in Cell Biology, 18, 565-571.
Kruse, P. H., J. Matta, S. Ugolini & E. Vivier (2014) Natural cytotoxicity receptors and their ligands. Immunol Cell Biol, 92, 221-9.
Lanier, L. L. (2015) NKG2D Receptor and Its Ligands in Host Defense. Cancer Immunol Res, 3, 575-82.
Lee, J. H. & S.-W. Lee (2017) The Roles of Carcinoembryonic Antigen in Liver Metastasis and Therapeutic Approaches. Gastroenterology Research and Practice, 2017, 11.
Lee, K.-A., E.-A. Bae, Y. C. Song, E.-K. Kim, Y.-S. Lee, T.-G. Kim & C.-Y. Kang (2015) A multimeric carcinoembryonic antigen signal inhibits the activation of human T cells by a SHP-independent mechanism: A potential mechanism for tumor-mediated suppression of T-cell immunity. International Journal of Cancer, 136, 2579-2587.
Lefranc, M. P., V. Giudicelli, C. Ginestoux, J. Bodmer, W. Muller, R. Bontrop, M. Lemaitre, A. Malik, V. Barbie & D. Chaume (1999) IMGT, the international ImMunoGeneTics database. Nucleic Acids Res, 27, 209-12.
Liersch, T., J. Meller, M. Bittrich, B. Kulle, H. Becker & D. M. Goldenberg (2007) Update of carcinoembryonic antigen radioimmunotherapy with (131)I-labetuzumab after salvage resection of colorectal liver metastases: comparison of outcome to a contemporaneous control group. Ann Surg Oncol, 14, 2577-90.
Li, Y., Q. Wang & R. A. Mariuzza (2011) Structure of the human activating natural cytotoxicity receptor CD137 bound to its tumor cell ligand B7-H6. J Exp Med, 208, 703-14.
Liu, L., W. Zeng, M. A. Wortinger, S. B. Yan, P. Cornwell, V. L. Peek, J. R. Stephens, J. W. Tetreault, J. Xia, J. R. Manro, K. M. Credille, D. W. Ballard, P. Brown-Augsburger, V. Wacheck, C. K. Chow, L. Huang, Y. Wang, I. Denning, J. Davies, Y. Tang, P. Vaillancourt & J. Lu (2014) LY2875358, a neutralizing and internalizing anti-MET bivalent antibody, inhibits HGF-dependent and HGF-independent MET activation and tumor growth. Clin Cancer Res, 20, 6059-70.
Matsuoka, Y., Y. Matsuo, K. Sugano, H. Ohkura, M. Kuroki & M. Kuroki (1990) Characterization of Carcinoembryonic Antigen-related Antigens in Normal Adult Feces. Japanese Journal of Cancer Research, 81, 514-519.
Mechetner, E. (2007) Development and characterization of mouse hybridomas. Methods Mol Biol, 378, 1-13.
Moertel, C. G., T. R. Fleming, J. S. Macdonald, D. G. Haller, J. A. Laurie & C. Tangen (1993) An evaluation of the carcinoembryonic antigen (CEA) test for monitoring patients with resected colon cancer. Jama, 270, 943-7.
Muller, D. & R. E. Kontermann (2010) Bispecific antibodies for cancer immunotherapy: Current perspectives. BioDrugs, 24, 89-98.
Nap, M., M. L. Hammarstrom, O. Bormer, S. Hammarstrom, C. Wagener, S. Handt, M. Schreyer, J. P. Mach, F. Buchegger, S. von Kleist & et al., (1992) Specificity and affinity of monoclonal antibodies against carcinoembryonic antigen. Cancer Res, 52, 2329-39.
Nap, M., K. Mollgard, P. Burtin & G. J. Fleuren (1988) Immunohistochemistry of carcino-embryonic antigen in the embryo, fetus and adult. Tumour Biol, 9, 145-53.
Oberst, M. D., S. Fuhrmann, K. Mulgrew, M. Amann, L. Cheng, P. Lutterbuese, L. Richman, S. Coats, P. A. Baeuerle & S. A. Hammond (2014) CEA/CD3 bispecific antibody MEDI-565/AMG 211 activation of T cells and subsequent killing of human tumors is independent of mutations commonly found in colorectal adenocarcinomas. MAbs, 6, 1571-84.
Pegram, H. J., D. M. Andrews, M. J. Smyth, P. K. Darcy & M. H. Kershaw (2011) Activating and inhibitory receptors of natural killer cells. Immunol Cell Biol, 89, 216-24.
Pende, D., S. Parolini, A. Pessino, S. Sivori, R. Augugliaro, L. Morelli, E. Marcenaro, L. Accame, A. Malaspina, R. Biassoni, C. Bottino, L. Moretta & A. Moretta (1999) Identification and molecular characterization of CD137, a novel triggering receptor involved in natural cytotoxicity mediated by human natural killer cells. J Exp Med, 190, 1505-16.
Peng, L., M. D. Oberst, J. Huang, P. Brohawn, C. Morehouse, K. Lekstrom, P. A. Baeuerle, H. Wu, Y. Yao, S. R. Coats, W. Dall'Acqua, M. Damschroder & S. A. Hammond (2012) The CEA/CD3-bispecific antibody MEDI-565 (MT111) binds a nonlinear epitope in the full-length but not a short splice variant of CEA. PloS one, 7, e36412-e36412.
Pesce, S., G. Tabellini, C. Cantoni, O. Patrizi, D. Coltrini, F. Rampinelli, J. Matta, E. Vivier, A. Moretta, S. Parolini & E. Marcenaro (2015) B7-H6-mediated downregulation of CD137 in NK cells contributes to ovarian carcinoma immune escape. Oncoimmunology, 4, e1001224.
Pogge von Strandmann, E., V. R. Simhadri, B. von Tresckow, S. Sasse, K. S. Reiners, H. P. Hansen, A. Rothe, B. Boll, V. L. Simhadri, P. Borchmann, P. J. McKinnon, M. Hallek & A. Engert (2007) Human leukocyte antigen-B-associated transcript 3 is released from tumor cells and engages the CD137 receptor on natural killer cells. Immunity, 27, 965-74.
Poul, M. A., B. Becerril, U. B. Nielsen, P. Morisson & J. D. Marks (2000) Selection of tumor-specific internalizing human antibodies from phage libraries. J Mol Biol, 301, 1149-61.
Primrose, J. N., R. Perera, A. Gray, P. Rose, A. Fuller, A. Corkhill, S. George & D. Mant (2014) Effect of 3 to 5 years of scheduled CEA and CT follow-up to detect recurrence of colorectal cancer: the FACS randomized clinical trial. Jama, 311, 263-70.
Richman, P. I. & W. F. Bodmer (1987) Monoclonal antibodies to human colorectal epithelium: markers for differentiation and tumour characterization. Int J Cancer, 39, 317-28.
Ridgway, J. B., L. G. Presta & P. Carter (1996) ‘Knobs-into-holes’ engineering of antibody CH3 domains for heavy chain heterodimerization. Protein Eng, 9, 617-21.
Saito, G., S. Sadahiro, K. Okada, A. Tanaka, T. Suzuki & A. Kamijo (2016) Relation between Carcinoembryonic Antigen Levels in Colon Cancer Tissue and Serum Carcinoembryonic Antigen Levels at Initial Surgery and Recurrence. Oncology, 91, 85-9.
Sakurai, Y., S. Hirohashi, T. Ohishi, Y. Shimosato, S. Kodaira & O. Abe (1989) Conformational epitopes specific to carcinoembryonic antigen defined by monoclonal antibodies raised against colon cancer xenografts. J Surg Oncol, 42, 39-46.
Sheahan, K., M. J. O'Brien, B. Burke, P. A. Dervan, J. C. O'Keane, L. S. Gottlieb & N. Zamcheck (1990) Differential reactivities of carcinoembryonic antigen (CEA) and CEA-related monoclonal and polyclonal antibodies in common epithelial malignancies. Am J Clin Pathol, 94, 157-64.
Shinmi, D., R. Nakano, K. Mitamura, M. Suzuki-Imaizumi, J. Iwano, Y. Isoda, J. Enokizono, Y. Shiraishi, E. Arakawa, K. Tomizuka & K. Masuda (2017) Novel anticarcinoembryonic antigen antibody-drug conjugate has antitumor activity in the existence of soluble antigen. Cancer Med, 6, 798-808.
Silacci, M., S. Brack, G. Schirru, J. Marlind, A. Ettorre, A. Merlo, F. Viti & D. Neri (2005) Design, construction, and characterization of a large synthetic human antibody phage display library. Proteomics, 5, 2340-50.
Stern, N., G. Markel, T. I. Amon, R. Gruda, H. Wong, S. D. Gray-Owen & O. Mandelboim (2005) Carcinoembryonic Antigen (CEA) Inhibits NK Killing via Interaction with CEA-Related Cell Adhesion Molecule 1. The Journal of Immunology, 174, 6692-6701.
Stewart, L. M., S. Young, G. Watson, S. J. Mather, P. A. Bates, H. A. Band, R. W. Wilkinson, E. L. Ross & D. Snary (1999) Humanisation and characterisation of PR1A3, a monoclonal antibody specific for cell-bound carcinoembryonic antigen. Cancer Immunol Immunother, 47, 299-306.
Takahashi, T., S. Minami, Y. Tsuchiya, K. Tajima, N. Sakai, K. Suga, S. I. Hisanaga, N. Ohbayashi, M. Fukuda & H. Kawahara (2019) Cytoplasmic control of Rab family small GTPases through BAG6. EMBO Rep, 20.
Thompson, J. A., F. Grunert & W. Zimmermann (1991) Carcinoembryonic antigen gene family: Molecular biology and clinical perspectives. Journal of Clinical Laboratory Analysis, 5, 344-366.
Von Kreudenstein, T. S., E. Escobar-Carbrera, P. I. Lario, I. D'Angelo, K. Brault, J. Kelly, Y. Durocher, J. Baardsnes, R. J. Woods, M. H. Xie, P. A. Girod, M. D. Suits, M. J. Boulanger, D. K. Poon, G. Y. Ng & S. B. Dixit (2013) Improving biophysical properties of a bispecific antibody scaffold to aid developability: quality by molecular design. MAbs, 5, 646-54.
Wong, J. Y., D. Z. Chu, L. E. Williams, D. M. Yamauchi, D. N. Ikle, C. S. Kwok, A. Liu, S. Wilczynski, D. Colcher, P. J. Yazaki, J. E. Shively, A. M. Wu & A. A. Raubitschek (2004) Pilot trial evaluating an 123I-labeled 80-kilodalton engineered anticarcinoembryonic antigen antibody fragment (cT84.66 minibody) in patients with colorectal cancer. Clin Cancer Res, 10, 5014-21.
Wu, J., Y. Song, A. B. Bakker, S. Bauer, T. Spies, L. L. Lanier & J. H. Phillips (1999) An activating immunoreceptor complex formed by NKG2D and DAP10. Science, 285, 730-2.
Wu, T. T. & E. A. Kabat (1970) An analysis of the sequences of the variable regions of Bence Jones proteins and myeloma light chains and their implications for antibody complementarity. J Exp Med, 132, 211-50.
Zhang, T., B. A. Lemoi & C. L. Sentman (2005) Chimeric NK-receptor-bearing T cells mediate antitumor immunotherapy. Blood, 106, 1544-1551.
Zhang, T., X. Song, L. Xu, J. Ma, Y. Zhang, W. Gong, Y. Zhang, X. Zhou, Z. Wang, Y. Wang, Y. Shi, H. Bai, N. Liu, X. Yang, X. Cui, Y. Cao, Q. Liu, J. Song, Y. Li, Z. Tang, M. Guo, L. Wang & K. Li (2018) The binding of an anti-PD-1 antibody to FcgammaRIota has a profound impact on its biological functions. Cancer Immunol Immunother, 67, 1079-1090.
Zhang, T., M. R. Wu & C. L. Sentman (2012) An CD137-based chimeric antigen receptor promotes T cell effector functions and antitumor efficacy in vivo. J Immunol, 189, 2290-9.
Zhao, L., L. Qu, J. Zhou, Z. Sun, H. Zou, Y. Y. Chen, J. D. Marks & Y. Zhou (2014) High throughput identification of monoclonal antibodies to membrane bound and secreted proteins using yeast and phage display. PLoS One, 9, e111339.

1. A multispecific antibody or antigen-binding fragment thereof, comprising a first antigen binding domain that specifically binds to human CEA at amino acids 596 to 674 of SEQ ID NO:88 and a second antigen binding domain that specifically binds to human CD137.

2. The multispecific antibody or antigen-binding fragment thereof of claim 1, wherein the first antigen binding domain does not bind to other CEACAM family members.

3. The multispecific antibody or antigen-binding fragment thereof of claim 1, wherein the first antigen binding domain that specifically binds to human CEA comprises:
(i) a heavy chain variable region that comprises (a) a HCDR1 of SEQ ID NO: 7, (b) a HCDR2 of SEQ ID NO:8, and (c) a HCDR3 of SEQ ID NO:9; and a light chain variable region that comprises: (d) a LCDR1 of SEQ ID NO: 10, (e) a LCDR2 of SEQ ID NO: 11, and (f) a LCDR3 of SEQ ID NO:6;
(ii) a heavy chain variable region that comprises (a) a HCDR1 of SEQ ID NO:24, (b) a HCDR2 of SEQ ID NO:25, and (c) a HCDR3 of SEQ ID NO:26; and a light chain variable region that comprises: (d) a LCDR1 of SEQ ID NO:27, (e) a LCDR2 of SEQ ID NO:28, and (f) a LCDR3 of SEQ ID NO: 23; or
(iii) a heavy chain variable region that comprises (a) a HCDR1 of SEQ ID NO:41, (b) a HCDR2 of SEQ ID NO:42, and (c) a HCDR3 of SEQ ID NO:43; and a light chain variable region that comprises: (d) a LCDR1 of SEQ ID NO:44, (e) a LCDR2 of SEQ ID NO:45, and (f) a LCDR3 of SEQ ID NO:40.

4. The multispecific antibody or antigen-binding fragment thereof of claim 3, wherein the first antigen binding domain that specifically binds to human CEA comprises:
(i) a heavy chain variable region comprising an amino acid sequence at least 90% identical to SEQ ID NO: 14, and a light chain variable region comprising an amino acid sequence at least 90% identical to SEQ ID NO: 15;
(ii) a heavy chain variable region comprising an amino acid sequence at least 90% identical to SEQ ID NO: 31, and a light chain variable region comprising an amino acid sequence at least 90% identical to SEQ ID NO: 32; or
(iii) a heavy chain variable region comprising an amino acid sequence at least 90% identical to SEQ ID NO: 48, and a light chain variable region comprising an amino acid sequence at least 90% identical to SEQ ID NO: 49.

5. (canceled)

6. The multispecific antibody or antigen-binding fragment thereof of claim 1, wherein the first antigen binding domain that specifically binds to human CEA comprises:
(i) a heavy chain variable region that comprises SEQ ID NO:14, and a light chain variable region that comprises SEQ ID NO: 15;
(ii) a heavy chain variable region that comprises SEQ ID NO: 31, and a light chain variable region that comprises SEQ ID NO: 32; or
(iii) a heavy chain variable region that comprises SEQ ID NO: 48, and a light chain variable region that comprises SEQ ID NO: 49.

7. The multispecific antibody or antigen-binding fragment thereof of claim 1, wherein the second antigen binding domain that specifically binds to human CD137 comprises:
(i) a heavy chain variable region that comprises (a) a HCDR1 of SEQ ID NO: 65, (b) a HCDR2 of SEQ ID NO:80, and (c) a HCDR3 of SEQ ID NO:81;
(ii) a heavy chain variable region that comprises (a) a HCDR1 of SEQ ID NO:65, (b) a HCDR2 of SEQ ID NO:73, and (c) a HCDR3 of SEQ ID NO:67;
(iii) a heavy chain variable region that comprises (a) a HCDR1 of SEQ ID NO:65, (b) a HCDR2 of SEQ ID NO:66, and (c) a HCDR3 of SEQ ID NO:67; or
(iv) a heavy chain variable region that comprises (a) a HCDR1 of SEQ ID NO:55, (b) a HCDR2 of SEQ ID NO:56, and (c) a HCDR3 of SEQ ID NO:57.

8. The multispecific antibody or antigen-binding fragment thereof of claim 7, wherein the second antigen binding domain that specifically binds to human CD137 comprises:
(i) a heavy chain variable region comprising an amino acid sequence at least 90% identical to SEQ ID NO:84;
(ii) a heavy chain variable region comprising an amino acid sequence at least 90% identical to SEQ ID NO: 86;
(iii) a heavy chain variable region comprising an amino acid sequence at least 90% identical to SEQ ID NO:75;
(iv) a heavy chain variable region comprising an amino acid sequence at least 90% identical to SEQ ID NO:70; or
(v) a heavy chain variable region comprising an amino acid sequence at least 90% identical to SEQ ID NO:60.

9. (canceled)

10. The multispecific antibody or antigen-binding fragment thereof of claim 1, wherein the second antigen binding domain that specifically binds to human CD137 comprises:
(i) a heavy chain variable region that comprises SEQ ID NO:84;
(ii) a heavy chain variable region that comprises SEQ ID NO: 86;
(iii) a heavy chain variable region that comprises SEQ ID NO: 75;
(iv) a heavy chain variable region that comprises SEQ ID NO: 70; or
(v) a heavy chain variable region that comprises SEQ ID NO: 60.

11. The multispecific antibody or antigen-binding fragment thereof of claim 1, wherein:
(i) the first antigen binding domain that specifically binds to human CEA comprises: a heavy chain variable region that comprises (a) a HCDR1 of SEQ ID NO: 7, (b) a HCDR2 of SEQ ID NO:8, and (c) a HCDR3 of SEQ ID NO:9; and a light chain variable region that comprises: (d) a LCDR1 of SEQ ID NO: 10, (e) a LCDR2 of SEQ ID NO: 11, and (f) a LCDR3 of SEQ ID NO:6; and the second antigen binding domain that specifically binds to human CD137 comprises: a heavy chain variable region that comprises (a) a HCDR1 of SEQ ID NO: 65, (b) a HCDR2 of SEQ ID NO:80, and (c) a HCDR3 of SEQ ID NO:81 and;
(ii) the first antigen binding domain that specifically binds to human CEA comprises: a heavy chain variable region that comprises (a) a HCDR1 of SEQ ID NO:7, (b) a HCDR2 of SEQ ID NO:8, and (c) a HCDR3 of SEQ ID NO:9; and a light chain variable region that comprises: (d) a LCDR1 of SEQ ID NO:10, (e) a LCDR2 of SEQ ID NO:11, and (f) a LCDR3 of SEQ ID NO: 6; and the second antigen binding domain that specifically binds to human CD137 comprises: a heavy chain variable region that comprises (a) a HCDR1 of SEQ ID NO: 65, (b) a HCDR2 of SEQ ID NO:73, and (c) a HCDR3 of SEQ ID NO:67;
(iii) the first antigen binding domain that specifically binds to human CEA comprises: a heavy chain variable region that comprises (a) a HCDR1 of SEQ ID NO:7, (b) a HCDR2 of SEQ ID NO:8, and (c) a HCDR3 of SEQ ID NO:9; and a light chain variable region that comprises: (d) a LCDR1 of SEQ ID NO:10, (e) a LCDR2 of SEQ ID NO:11, and (f) a LCDR3 of SEQ ID NO:6; and the second antigen binding domain that specifically binds to human CD137 comprises: a heavy chain variable region that comprises (a) a HCDR1 of SEQ ID NO: 65, (b) a HCDR2 of SEQ ID NO:66, and (c) a HCDR3 of SEQ ID NO:67; or
(iv) the first antigen binding domain that specifically binds to human CEA comprises: a heavy chain variable region that comprises (a) a HCDR1 of SEQ ID NO: 7, (b) a HCDR2 of SEQ ID NO:8, and (c) a HCDR3 of SEQ ID NO:9; and a light chain variable region that comprises: (d) a LCDR1 of SEQ ID NO:10, (e) a LCDR2 of SEQ ID NO:11, and (f) a LCDR3 of SEQ ID NO:6; and the second antigen binding domain that specifically binds to human CD137 comprises: a heavy chain variable region that comprises (a) a HCDR1 of SEQ ID NO:55, (b) a HCDR2 of SEQ ID NO:56, and (c) a HCDR3 of SEQ ID NO:57.

12. The multispecific antibody or antigen-binding fragment thereof of claim 1, wherein:
(i) the first antigen binding domain that specifically binds to human CEA comprises: a heavy chain variable region that comprises SEQ ID NO:14, and a light chain variable region that comprises SEQ ID NO: 15; and the second antigen binding domain that specifically binds to human CD137 comprises: a heavy chain variable region that comprises SEQ ID NO:84;
(ii) the first antigen binding domain that specifically binds to human CEA comprises: a heavy chain variable region that comprises SEQ ID NO:14, and a light chain variable region that comprises SEQ ID NO: 15; and the second antigen binding domain that specifically binds to human CD137 comprises: a heavy chain variable region that comprises SEQ ID NO:86;
(iii) the first antigen binding domain that specifically binds to human CEA comprises: a heavy chain variable region that comprises SEQ ID NO:14, and a light chain variable region that comprises SEQ ID NO: 15; and the second antigen binding domain that specifically binds to human CD137 comprises: a heavy chain variable region that comprises SEQ ID NO:75;
(iv) the first antigen binding domain that specifically binds to human CEA comprises: a heavy chain variable region that comprises SEQ ID NO:14, and a light chain variable region that comprises SEQ ID NO: 15; and the second antigen binding domain that specifically binds to human CD137 comprises: a heavy chain variable region that comprises SEQ ID NO:70; or
(v) the first antigen binding domain that specifically binds to human CEA comprises: a heavy chain variable region that comprises SEQ ID NO:14, and a light chain variable region that comprises SEQ ID NO: 15; and the second antigen binding domain that specifically binds to human CD137 comprises: a heavy chain variable region that comprises SEQ ID NO:60.

13. The multispecific antibody or antigen-binding fragment thereof of claim 1, wherein the multispecific antibody or antigen-binding fragment thereof is a monoclonal antibody, a chimeric antibody, a humanized antibody, a human engineered antibody, a single chain antibody (scFv), a Fab fragment, a Fab′ fragment, or a F(ab′)2 fragment.

14. The multispecific antibody or antigen-binding fragment thereof of claim 1, wherein the multispecific antibody or antigen-binding fragment thereof is a bispecific antibody.

15. The multispecific antibody or antigen-binding fragment thereof of claim 14, wherein the bispecific antibody comprises a linker selected from SEQ ID NO:317 to SEQ ID NO: 358.

16. The multispecific antibody or antigen-binding fragment thereof of claim 15, wherein the linker is SEQ ID NO: 324 or SEQ ID NO: 329.

17. (canceled)

18. The multispecific antibody or antigen-binding fragment thereof of claim 14, wherein the bispecific antibody is BE-146 (SEQ ID NO: 313 and SEQ ID NO: 179), BE-189 (SEQ ID NO: 255 and SEQ ID NO: 179), BE-718 (SEQ ID NO: 295 and SEQ ID NO: 179), BE-740 (SEQ ID NO: 297 and SEQ ID NO: 179), BE-942 (SEQ ID NO: 299, SEQ ID NO: 301, and SEQ ID NO:303), BE-755 (SEQ ID NO: 299, SEQ ID NO: 301, and SEQ ID NO: 305), BE-562 (SEQ ID NO: 307 and SEQ ID NO: 179), BE-375 (SEQ ID NO: 309 and SEQ ID NO: 179), or BE-244 (SEQ ID NO: 311 and SEQ ID NO: 179).

19.-26. (canceled)

27. The multispecific antibody or antigen-binding fragment thereof of claim 1, wherein the antibody or antigen-binding fragment thereof has one or more of the following: antibody dependent cellular cytotoxicity (ADCC), complement dependent cytotoxicity (CDC), reduced glycosylation, no glycosylation, hypofucosylation, or increased bisecting GlcNac structures.

28.-29. (canceled)

30. The multispecific antibody or antigen-binding fragment thereof of claim 1, wherein the Fc domain is an IgG1 with reduced effector function or an IgG4.

31. (canceled)

32. A pharmaceutical composition comprising the multispecific antibody or antigen-binding fragment thereof of claim 1 and a pharmaceutically acceptable carrier.

33. (canceled)

34. A method of treating cancer comprising administering to a patient in need an effective amount of the multispecific antibody or antigen-binding fragment thereof of claim 1.

35.-46. (canceled)

47. An isolated nucleic acid that encodes the multispecific antibody or antigen-binding fragment thereof of claim 1.

48. A vector comprising the nucleic acid of claim 47.

49. A host cell comprising the nucleic acid of claim 47.

50. A process for producing a multispecific antibody or antigen-binding fragment thereof comprising cultivating the host cell of claim 49 and recovering the antibody or antigen-binding fragment thereof from the culture.
